(19) United States
(12) Patent Application Publication
(10) Pub. No.: US 2025/0163167 A1
(43) Pub. Date: May 22, 2025

(54) IL10 RECEPTOR BINDING MOLECULES AND METHODS OF USE

(71) Applicant: Synthekine, Inc., Menlo Park, CA (US)

(72) Inventors: Robert Kastelein, Menlo Park, CA (US); Mahalakshmi Ramadass, Menlo Park, CA (US); Sandro Vivona, Menlo Park, CA (US); Deepti Rokkam, Menlo Park, CA (US); Patrick J. Lupardus, Menlo Park, CA (US)

(21) Appl. No.: 18/904,938

(22) Filed: Oct. 2, 2024

Related U.S. Application Data
(63) Continuation of application No. 18/164,351, filed on Feb. 3, 2023, now Pat. No. 12,139,545.
Application No. 18/164,351 is a continuation of application No. PCT/US21/44834, filed on Aug. 5, 2021.
(60) Provisional application No. 63/061,562, filed on Aug. 5, 2020.
(60) Provisional application No. 63/078,745, filed on Sep. 15, 2020.
(60) Provisional application No. 63/136,098, filed on Jan. 11, 2021.
(60) Provisional application No. 63/135,884, filed on Jan. 11, 2021.

(51) Int. Cl. C07K 16/28; A61K 45/06; C12N 15/63

(52) U.S. Cl. CPC C07K 16/2866; A61K 45/06; C12N 15/63; C07K 2317/22; C07K 2317/24; C07K 2317/31; C07K 2317/565; C07K 2317/569; C07K 2317/92

(57) ABSTRACT

Provided herein are IL10R binding molecules that bind to IL10Ra and IL10Rb and comprise an IL10Rb sdAb and an IL10Rb VHH antibody.

### CROSS-REFERENCES TO RELATED APPLICATIONS

This application is a continuation of U.S. application Ser. No. 18/164,351, filed Feb. 3, 2023, which is a continuation of International Patent Application No. PCT/US2021/044834, filed Aug. 5, 2021, which claims priority to U.S. Provisional Application No. 63/061,562, filed Aug. 5, 2020, U.S. Provisional Application No. 63/078,745, filed Sep. 15, 2020, U.S. Provisional Application No. 63/135,884, filed Jan. 11, 2021 and U.S. Provisional Application No. Ser. No. 63/136,098, filed on Jan. 11, 2021, the disclosures of which are hereby incorporated by reference in their entirety for all purposes.

### SEQUENCE LISTING

The instant application contains a Sequence Listing which has been submitted electronically in XML format and is hereby incorporated by reference in its entirety. Said XML file, created on Feb. 2, 2023, is named 106249-1362149-001131US_SL.xml, and is 865 bytes in size.

### BACKGROUND OF THE DISCLOSURE

Cytokine and growth-factor ligands typically signal through multimerization of cell surface receptors subunits. In some instance, cytokines act as multispecific (e.g., bispecific or trispecific) ligands which facilitate the association of such receptor subunits, bringing their intracellular domains into proximity such that intracellular signaling may occur. The nature of the cytokine determines which receptor subunits are associated to form the cytokine receptor complex. Cytokines thus act to bridge the individual receptor subunits into a receptor complex that results in intracellular signaling.

The intracellular domains of cytokine receptor subunits possess proline rich JAK binding domains which are typically located in the box1/box region of the intracellular domain of the cytokine receptor subunit near the interior surface of the cell membrane. Intracellular JAK kinases associate with JAK binding domains. When the intracellular domains receptor subunits are brought into proximity, typically by the binding of the cognate ligand for the receptor to the extracellular domains of the receptor subunts, the JAKs phosphorylate each other. Four Janus kinases have been identified in mammalian cells: JAK1, JAK2, JAK3 and TYK2. Ihle, et al. (1995) Nature 377(6550):591-4, 1995; O'Shea and Plenge (2012) Immunity 36(4):542-50. The phosphorylation of the JAK induces a conformational change in the JAK providing the ability to further phosphorylate other intracellular proteins which initiates a cascade that results in activation of multiple intracellular factors which transduce the intracellular signal associated with the receptor resulting intracellular responses such as gene transcription, frequently referred to as downstream signaling. In many instances, the proteins which are phosphorylated by the JAKs are members of the signal transducer and activator of transcription (STAT) protein family. Seven members of the mammalian STAT family have been identified to date: STAT1, STAT2, STAT3, STAT4, STAT5a STAT5b, and STAT6. Delgoffe, et al., (2011) Curr Opin Immunol. 23(5):632-8; Levy and Darnell (2002) Nat Rev Mol Cell Biol. 3(9):651-62 and Murray, (2007) J Immunol. 178(5):2623-9. The selective interplay of activated JAK and STAT proteins, collectively refererred to a the JAK/STAT pathway, provide for a wide variety of intracellular responses observed in response to cytokine binding.

The human genome encodes for approximately forty different JAK/STAT cytokine receptors. In principle, approximately 1600 unique homodimeric and heterodimeric cytokine receptor pairs could be generated with the potential to signal through different JAK/TYK/STAT combinations (Bazan, Proc Natl Acad Sci USA. 87(18):6934-8, 1990; Huising et al., J Endocrinol. 189(1):1-25, 2006). However, as of the present knowledge, the human genome encodes for less than fifty different cytokine ligands (Bazan, Proc Natl Acad Sci USA. 87(18):6934-8, 1990; Huising et al., J Endocrinol. 189(1):1-25, 2006), limiting the scope of cytokine receptor complexes to those that can be assembled by the natural ligands. Given that interaction of the a cytokine ligand with the extracellular domains of the cytokine receptor subunits determines the composition of receptor subunits in a receptor complex and the intracellular JAK/TYK and RTK enzymes are degenerate, the number of cytokine and growth factor receptor dimer pairings that occur in nature represents only a fraction of the total number of signaling-competent receptor pairings theoretically allowed by the system.

Naturally occurring cytokine ligands mediate a wide variety of cellular response. In some instances, a heteromultimeric cytokine receptor is composed of one or more receptor subunits that is unique to the receptor complex, referred to as “proprietary” subunits, which interact with other receptor subunits that are shared by multiple cytokine receptors, frequently referred to as “common” receptor subunits. For example, the IL7 receptor is a heterodimeric receptor complex of the IL7Ra proprietary subunit and a CD132 subunit which is also referred to as the “common gamma” subunit as it is a shared receptor subunit of multiple cytokine receptor complexes including IL2, IL4, IL19, IL15 and IL21. The relative affinity and kinetic of the interaction of the cytokine for the ECDs of the receptor subunits and the stability of the complex formed in response to cytokine binding mediates the nature and intensity of the intracellular signaling. In some instances, the binding of the cytokine to a the proprietary subunit enhances the formation of the complete receptor where the affinity of the cytokine for the common subunit may be significantly lower when not associated with the proprietary subunit.

The nuances of the interplay between the cytokine ligand and the receptor subunits is a matter of significant scientific investigation. For example, many properties of naturally occurring cytokines suggest their potential utility in the treatment of human disease but such naturally occurring cytokines may also trigger adverse and undesirable effects. In many instances, the disease is associated with a particular cell type which expresses the receptor for the potentially therapeutic cytokine. However, the cytokine receptor is also expressed on other cell types not desired to be targeted for therapeutic intervention. The administration of the native ligand activates both cell types resulting result in undesirable side effects.

To attempt to generate cytokine analogs which provide selective activation of the desired cell types, a variety of engineered cytokine ligands (or components thereof) have been generated so as to selectively modulate their affinity for the extracellular domains of receptor subunits. These efforts have generated cytokine variants been shown to provide partial activity which results in uncoupling of the beneficial properties of the ligand from the undesired effects. See, e.g, Mendoza, et al. (2019) 567:56-60. However, the engineering of such selective cytokines ligand is based on selective modulation of individual amino acid residues at the interface of the ligand and the receptor. This protein engineering approach to modulation of cytokine receptor affinity requires a three dimensional, usually x-ray crystallographic, map of the interation of the receptor and the cytokine to identify the residues of the cytokine that interface with the receptor subunit. Addtionally the effects of amino acid subsitutions at these interface residues can be highly variable often requiring a significant amount of time consuming trial-and-error to identify the particular amino acid substitutions required to produce the desired activity profile. However, even once the engineered cytokine with the desired signaling profile is achieved, many proteins are highly sensitive to amino acid substitutions result in significant issues for recombinant expression, both in mammalian expression systems and procaryotic systems where such amino acid substitutions can affect protein refolding when expressed in inclusion bodies. The foregoing issues are particularly acute when the cytokine itself is heterodimeric. For example, IL12 exists a heterodimer of the p35 and p40 subunits. The engineering of one residues of the interface of one subunit of the ligand engineering of the cytokine subunit at the interface with the receptor subunit (e.g. the p40/IL12Rb2 interface) may cause issues with the association of the cytokine ligand subunits (e.g. p35 and p40). Additionally, the p40 subunit of IL12 has functions as a monomer and homodimer whose activities may also be affected by such amino acid substitions. Consequently, there is a need in the art for molecules which provide the activity of cytokines, that may be readily generated and engineered for receptor affinity and do not have independent off-target activity.

### BRIEF DESCRIPTION OF THE FIGURES

FIG. 1 of the attached drawings provides a schematic representation of one embodiment of the binding molecule of the present disclosure comprising a first single domain antibody (1) and a second single domain antibody (3) and a linker (2) depicted as interacting with a cell membrane (10) associated heterodimeric receptor comprising a first receptor subunit comprising an extracellular domain (4), and transmembrane domain (5) and an intracellular domain (6) nteraction of a binding molecule and a second first receptor subunit comprising an extracellular domain (7), and transmembrane domain (8) and an intracellular domain (9) wherein the intracellular domain of the first receptor (6) and the intracellular domain of the second receptor (9) on of a binding molecule are within a proximal distance (11).

FIGS. 2A-2B of the attached drawings provides a schematic representation of two illustrative configurations of binding molecules of the present disclosure. FIG. 2A provides a schematic representation of an illustrative single polypeptide chain binding molecule comprising, from amino to carboxy, a first single domain antibody (1) and a second single domain antibody (3) and a linker (2). FIG. 2B provides a schematic representation of a binding molecule comprising a first single domain antibody (1) and a second single domain antibody (3) and a linker (2) and a knob-into-hole Fc domain comprising a first subunit which is a Fc knob (13) and a second subunit which is a Fc hole (14) wherein the single domain antibody is stably associated with the Fc domain via a IgG hinge sequence (12).

FIGS. 2A-2B of the attached drawings provides a schematic representation of two illustrative configurations of binding molecules of the present disclosure. FIG. 2A provides a schematic representation of an illustrative binding molecule comprising a first single domain antibody (1) and a second single domain antibody (3) and a linker (2). FIG. 2B provides a schematic representation of a binding molecule comprising two polypeptide chains, the first polypeptide chain comprising (from amino to carboxy) a first single domain antibody (1), a linker sequence, a second single domain antibody (3), an IgG hinge sequence (12) and an Fc knob domain (13) and a second polypeptide comprising an Fc hole (14) wherein the first and second polypeptides are in stable association via the interaction of the knob-into-hole Fc domain.

FIGS. 3A-3B of the attached drawings provides a schematic representations of two illustrative configurations of binding molecules of the present disclosure. FIG. 3A provides a schematic representation of an illustrative binding molecule construct comprising two binding molecules each attached to a subunit of a knob-into-hole Fc domain, the construct comprising two polypeptide chains, the first polypeptide chain comprising, from amino to carboxy, a first single domain antibody (1), a linker (2) and a second single domain antibody (3), a IgG hinge sequence (12) and a Fc knob subunit (13) and a second polypeptide chain comprising, from amino to carboxy, a first single domain antibody (1), a linker (2) and a second single domain antibody (3), a IgG hinge sequence (12) and a Fc hole subunit (14) wherein the first and second polypeptides are in stable associate via the interaction of the knob-into-hole Fc domain. FIG. 3B provides schematic representation of a an alternative arrangement of a binding molecule construct comprising two polypeptides a first polypeptide chain comprising, from amino to carboxy, a first single domain antibody (1), a linker (2) and a second single domain antibody (3), an IgG hinge sequence (12) and a Fc knob subunit (13) and a second polypeptide chain comprising, from amino to carboxy, a first second domain antibody (3), a linker (2) and a first single domain antibody (1), a IgG hinge sequence (12) and a Fc hole subunit (14), wherein the first and second polypeptides are in stable association via the interaction of the knob-into-hole Fc domain.

FIG. 4A provides alternative schematic representations of configurations of the binding molecules of the present disclosure where one single domain antibody is attached to each subunit of a knob-into-hole Fc domain comprising two polypeptides, the first polypeptide comprising from amino to carboxy, a first single domain antibody (1), an IgG hinge sequence (12) and a Fc knob subunit (13), the second polypeptide comprising from amino to carboxy, a second single domain antibody (3), an IgG hinge sequence (12) and a Fc hole subunit (13), wherein the first and second single domain antibodies are in stable associate via the interaction of the knob-into-hole Fc domain.

FIG. 4B provides a schematic representations of a binding molecule the binding domains are single domain antibodies associated via transition metal coordinate covalent complex. As illustrated, the binding molecules comprises two polypeptide subunits: the first subunit comprising a first single domain antibody (1) is attached via a first linker (15) to a first chelating peptide (17) and second subunit comprising a second single domain antibody (3) is attached via a second linker (16) to a second chelating peptide (18), wherein the first chelating peptide (17) and second chelating peptide (18) form a coordinate covalent complex with a single transition metal ion (“M”). The transition metal ion may be in a kinetically labile or kinetically inert oxidation state.

FIG. 5 of the attached drawings provides the schematic representation of the protocol used to evaluate IL10R binding molecules for the effect of linker length on activity in CD4 T cells, CD8 T cells and monocytes isolated from PBMCs.

FIGS. 6A-6C of the attached drawings provides data from the evaluation of IL10R polypeptide binding molecules and the ability to induce IL10 activity as measured by phosphor STAT3 (y-axis) and varying concentrations of the IL10R binding molecule test articles on the x-axis as indicated to evaluate variations in distance between the IL10Ra and IL10Rb sdAbs on IL10 activity. In all experiments, the n-terminal IL10Ra sdAb of the polypeptide IL10R binding molecules was DR241. In FIG. 6A, the C-terminal IL2Rb sdAb was DR244. In FIG. 6B, the C-terminal IL2Rb sdAb was DR246. In FIG. 6C, the C-terminal IL2Rb sdAb was DR247. Various lines represent IL10R binding molecules wherein the distance between the IL10Ra and IL10Rb sdAbs was varied with linker length.

### SUMMARY OF THE DISCLOSURE

The present disclosure provides compositions useful in the pairing of cellular receptors to generate desirable effects useful in treatment of disease in mammalian subjects.

The present disclosure provides compositions comprising at least a first domain that specifically binds to a first receptor subunit and a second domain that specifically binds to a second receptor subunit, such that upon contacting with a cell expressing the first and second receptors, the composition causes the functional association of the first and second receptors, thereby triggering their interaction and resulting in downstream signaling. In some embodiments, the first and second receptors occur in proximity in response to the cognate ligand binding and are referred to herein as “natural” cytokine receptor pairs.

In one embodiment, present disclosure provides binding molecules that comprise a first domain that binds to IL10Ra of the IL10 receptor and a second domain that binds to IL10Rb of the IL10 receptor, such that upon contacting with a cell expressing IL10Ra receptor and IL10Rb receptor, the IL10R binding molecule causes the functional association of IL10Ra and IL10Rb, thereby resulting in functional dimerization of the receptors and downstream signaling.

The present disclosure provides disclosure provides cytokine receptor binding molecules that are ligands for a cytokine receptor, the cytokine receptor binding molecule comprising:
(a) a first single domain antibody (sdAb) that specifically binds to the extracellular domain a first subunit of a cytokine receptor; and
(b) a second single domain antibody that specifically binds to extracellular domain of a second subunit of cytokine receptor subunit;
   wherein:
the first sdAb and second sdAb are in stable association;
the first and second subunits of the cytokine receptor are dimerized in response to contact with the cognate ligand for the cytokine receptor; and
contacting a cell expressing the first and the second subunist of the cytokine receptor with an effective amount of the cytokine receptor binding molecule results in the intracellular domains of the first and second subunits of the cytokine receptors being brought into proximity and results in intracellular signaling.

The IL10R binding molecules of the present disclosure comprise two or more single domain antibodies that selectively bind to the extracellular domain of the IL10Ra and IL10Rb receptor subunits. In one embodiment, the present disclosure provides an IL10 receptor (IL10R) binding molecule that is a ligand for the IL10R receptor, the IL10R receptor binding molecule comprising:
(c) a first single domain antibody (sdAb) that specifically binds to the extracellular domain the IL10Ra subunit of the IL10 receptor (an “IL10Ra sdAb”), and
(d) a second single domain antibody that specifically binds to the extracellular domain of the IL10Rb subunit of the IL10 receptor (an “IL10Rb sdAb”),
   wherein:
the first sdAb and second sdAb are in stable association;
the IL10Ra and IL10Rb subunits of the IL10 receptor are dimerized in response to contact with the IL10R binding molecule; and
contacting a cell expressing the IL10Ra and IL10Rb with an effective amount of the IL10R binding molecule results in the intracellular domains of IL10Ra and IL10Rb being brought into proximity and intracellular signaling.

In one embodiment, the cytokine receptor binding molecule is a ligand for the IL10 receptor and comprises a first sdAb that specifically binds to the extracellular domain of the IL10Ra receptor subunit and a second sdAb that specifically binds to the extracellular domain of the IL10Rb receptor subunit.

In some embodiments, the anti-IL10Rα sdAb is a VHH antibody (an anti IL10Rα VHH antibody) and/or the anti-IL10Rβ sdAb is a VHH antibody (an anti IL10Rβ VHH antibody). In some embodiments, the anti-IL10Rα sdAb and the anti-IL10Rβ sdAb are joined by a peptide linker. In some embodiments, the peptide linker comprises between 1 and 50 amino acids.

In another aspect, the disclosure provides a method for treating neoplastic diseases, such as cancer in a subject in need thereof, comprising administering to the subject the IL10R binding protein described herein, wherein the IL10R binding protein binds to and activates CD8⁺ T cells, CD4⁺ T cells, macrophages, and/or Treg cells. In some embodiments, the IL10R binding protein provides longer therapeutic efficacy than a pegylated IL10. In some embodiments, the cancer is a solid tumor cancer.

In other aspects, the IL10R binding proteins described herein can als be used to treat inflammatory diseases, such as Crohn's disease and ulcerative colitis, and autoimmune diseases, such as psoriasis, rheumatoid arthritis, and multiple sclerosis.

In some embodiments, one sdAb of the binding molecule is an scFv and the other sdAb is a VHH.

In some embodiments, the first and second sdAbs are covalently bound via a chemical linkage.

In some embodiments, the first and second sdAbs are provided as single continuous polypeptide.

The present invention provides IL10R binding molecules that are synthetic ligands of the IL10 receptor.

In some embodiments, the IL10R binding molecule of the present invention is a polypeptide of the following formula [#1]:

H2N-(IL10 VHH #1)-(L1)ₐ-(IL10 VHH #2)-(L2)_{b}-(CP)_{c}-COOH [#1]

wherein: “-” represents a covalent bond; L1 and L2 are linkers; CP is a chelating peptide; a, b, and c are independently selected from the integers 0 or 1; “H2N” denotes the amino terminus; and “COOH” denotes the carboxy terminus of the polypeptide.

In some embodiments the present invention provides the IL10R binding molecule of the present invention is a polypeptide of the following formula [#1], wherein IL10 VHH #1 is an ILRa sdAb and IL10 VHH #2 is an IL10Rb sdAb, IL10 VHH #1 is an ILRb sdAb and IL10 VHH #2 is an IL10Ra sdAb.

In some embodiments, the invention provides and IL10R binding molecule comprising a IL10Ra sdAb comprising:
a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 1, 4, 7, 10, 13, 16, 19, 22, 25, 28, 31, 34, 37, 40, 43, 46, 49, and 52;
a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 2, 5, 8, 11, 14, 17, 20, 23, 26, 29, 32, 35, 38, 41, 44, 47, 50 and 53; and
a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 3, 6, 9, 12, 15, 18, 21, 24, 27, 30, 33, 36, 39, 42, 45, 48, 51 and 54.

In some embodiments, the invention provides and IL10R binding molecule comprising a IL10Rb sdAb comprising:
a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148 and 151;
a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149 and 152; and
a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150 and 153.

In some embodiments, the invention provides and IL10R binding molecule comprising a IL10Ra sdAb having least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative to any one of SEQ ID NOS: 154-171.

In some embodiments, the invention provides and IL10R binding molecule comprising a IL10Rb sdAb having least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative to any one of SEQ ID NOS: 172-198 or SEQ ID Nos:199-201.

In some embodiments, the invention provides and IL10R binding molecule comprising a having least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative to any one of SEQ ID NOS: 256-353.

In some embodiments, the invention provides and IL10R binding molecule comprising a IL10Rb sdAb having least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative to any one of SEQ ID NOS: 172-198 or SEQ ID Nos:199-201.

In some embodiments, the present invention provides IL10R binding molecules having at least 90% (e.g., 91%, 92%, 93%, 94%, 95%, 96%, 97%, 98%, 99%, or 100%) sequence identity to the sequence of any one of the IL10R binding molecules of Table 29 SEQ ID NOS: [DR1511-DR1525]. In some embodiments, the present invention provides IL10R binding molecules substantially identical of any one of the IL10R binding molecules of Table 29 SEQ ID NOS: [DR1511-DR1525]. In some embodiments, the present invention provides IL10R binding molecules identical to a sequence of any one of the IL10R binding molecules of Table 29 SEQ ID NOS: [DR1511-DR1525].

In one embodiment, the present disclosure provides an IL10Ra binding molecule that preferentially activates T cells, in particular CD8+ T cells, relative to monocytes. In one embodiment, the present disclosure provides an IL10Ra binding molecule of the formula #1 wherein the affinity of the IL10Ra sdAb has a higher affinity for the extracellular domain of IL10Ra than the affinity of the IL10Rb sdAb for the extracellular domain of IL10Rb.

In some embodiments, the present disclosure provides an IL10R binding molecule modified to provide prolonged duration of action in vivo in a mammalian subject and pharmaceutically acceptable formulations thereof. In some embodiments, the present invention provides a IL10R binding molecules of formula #1 that is PEGylated, wherein the PEG is conjuaged to the IL10R binding molecule and the PEG is a linear or branched PEG molecule having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

The IL10R binding molecules of the present disclosure are useful in the treatment or prevention of disease in mammalian subjects. In some embodiments, the present disclosure provides for the treatment or prevention of autoimmune disease in a mammalian subject by the administration of a therapeutically effective amount of an IL10R binding molecule of the present disclosure. In some embodiments, the present disclosure provides for the treatment or prevention of infectious disease, including viral and chronic viral infections, in a mammalian subject by the administration of a therapeutically effective amount of an IL10R binding molecule of the present disclosure. In some embodiments, the present disclosure provides for the treatment or prevention of neoplastic disease in a mammalian subject by the administration of a therapeutically effective amount of an IL10R binding molecule of the present disclosure. In some embodiments, the present disclosure provides for the treatment or prevention of neoplastic, infectious or autoimmune in a mammalian subject by the administration of a therapeutically effective amount of an IL10R binding molecule of the present disclosure in combination with one or more supplementary therapeutic agents.

The present disclosure further provides a pharmaceutically acceptable formulation of an IL10R binding molecule for the administration to a mammalian subject. The present disclosure further provides a pharmaceutically acceptable composition for administration to a mammalian subject the composition comprising a nucleic acid sequence encoding a polypeptide IL10R binding molecule, a recombinant viral or non-viral vector encoding or polypeptide IL10R binding molecule, or a recombinantly modified mammalian cell comprising a nucleic acid sequence encoding a polypeptide IL10R binding molecule, in each case the nucleic acid sequence operably linked to one or more expression control elements functional in a mammalian cell.

The present disclosure provides nucleic acid sequences encoding polypeptide IL10R binding molecules. The present disclosure further provides a recombinant vector comprising a nucleic acid sequence encoding polypeptide IL10R binding molecules. The present disclosure further provides a recombinantly modified mammalian cell comprising a nucleic acid encoding a polypeptide IL10R binding molecule. The present disclosure further provides methods for the recombinant production, isolation, purification and characterization of a polypeptide IL10R binding molecule. of recombinant vectors comprising a provides nucleic acid sequences encoding polypeptide IL10R binding molecules.

The disclosure also provides an expression vector comprising a nucleic acid encoding the bispecific binding molecule operably linked to one or more expression control sequences. The disclosure also provides an isolated host cell comprising the expression vector expression vector comprising a nucleic acid encoding the bispecific binding molecule operably linked to one or more expression control sequences functional in the host cell.

In another aspect, the disclosure provides a pharmaceutical composition comprising the IL10R binding molecule described herein and a pharmaceutically acceptable carrier.

In another aspect, the disclosure provides a method of treating an autoimmune or inflammatory disease, disorder, or condition or a viral infection in a subject in need thereof, comprising administering to the subject a therapeutically effective amount of an IL10R binding molecule described herein or a pharmaceutical composition described herein.

Several advantages flow from the binding molecules described herein. The natural ligand of the IL10 receptor, IL10, causes IL10Ra and IL10Rb to come into proximity (i.e., by their simultaneous binding of IL10). However, when IL10 is used as a therapeutic in mammalian, particularly human, subjects, it may also trigger a number of adverse and undesirable effects by a variety of mechanisms including the presence of IL10Ra and IL10Rb on other cell types and the binding to IL10Ra and IL10Rb on the other cell types may result in undesirable effects and/or undesired signaling on cells expressing IL10Ra and IL10Rb. The present disclosure is directed to methods and compositions that modulate the multiple effects of IL10Ra and IL10Rb binding so that desired therapeutic signaling occurs, particularly in a desired cellular or tissue subtype, while minimizing undesired activity and/or intracellular signaling.

In some embodiments, the IL10R binding molecules described herein are partial agonists of the IL10 receptor. In some embodiments, the binding molecules described herein are designed such that the binding molecules are full agonists. In some embodiments, the binding molecules described herein are designed such that the binding molecules are super agonists.

In some embodiments, the binding molecules provide the maximal desired IL10 intracellular signaling from binding to IL10Ra and IL10Rb on the desired cell types, while providing significantly less IL10 signaling on other undesired cell types. This can be achieved, for example, by selection of binding molecules having differing affinities or causing different Emax for IL10Ra and IL10Rb as compared to the affinity of IL10 for IL10Ra and IL10Rb. Because different cell types respond to the binding of ligands to its cognate receptor with different sensitivity, by modulating the affinity of the dimeric ligand (or its individual binding moieties) for the IL10 receptor relative to wild-type IL10 binding facilitates the stimulation of desired activities while reducing undesired activities on non-target cells.

### DETAILED DESCRIPTION OF THE INVENTION

To facilitate the understanding of present disclosure, certain terms and phrases are defined below as well as throughout the specification. The definitions provided herein are non-limiting and should be read in view of the knowledge of one of skill in the art would know.

Before the present methods and compositions are described, it is to be understood that this invention is not limited to a particular method or composition described, as such may, of course, vary. It is also to be understood that the terminology used herein is for the purpose of describing embodiments only and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); pl=picoliter(s); s or sec=second(s); min=minute(s); h or hr=hour(s); AA or aa=amino acid(s); kb=kilobase(s); nt=nucleotide(s); pg=picogram; ng=nanogram; μg=microgram; mg=milligram; g=gram; kg=kilogram; dl or dL=deciliter; μl or μL=microliter; ml or mL=milliliter; l or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=once weekly; QM=once monthly; HPLC=high performance liquid chromatography; BW=body weight; U=unit; ns=not statistically significant; PBS=phosphate-buffered saline; PCR=polymerase chain reaction; HSA=human serum albumin; MSA=mouse serum albumin; DMEM=Dulbeco's Modification of Eagle's Medium; EDTA=ethylenediaminetetraacetic acid.

It will be appreciated that throughout this disclosure reference is made to amino acids according to the single letter or three letter codes. For the reader's convenience, the single and three letter amino acid codes are provided in Table 1.

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

### Definitions

Unless otherwise indicated, the following terms are intended to have the meaning set forth below. Other terms are defined elsewhere throughout the specification.

Activate: As used herein the term “activate” is used in reference to a receptor or receptor complex to reflect a biological effect, directly and/or by participation in a multicomponent signaling cascade, arising from the binding of an agonist ligand to a receptor responsive to the binding of the ligand.

Activity: As used herein, the term “activity” is used with respect to a molecule to describe a property of the molecule with respect to a test system (e.g. an assay) or biological or chemical property (e.g. the degree of binding of the molecule to another molecule) or of a physical property of a material or cell (e.g. modification of cell membrane potential). Examples of such biological functions include but are not limited to catalytic activity of a biological agent, the ability to stimulate intracellular signaling, gene expression, cell proliferation, the ability to modulate immunological activity such as inflammatory response. “Activity” is typically expressed as a level of a biological activity per unit of agent tested such as [catalytic activity]/[mg protein], [immunological activity]/[mg protein], international units (IU) of activity, [STAT5 phosphorylation]/[mg protein], [T-cell proliferation]/[mg protein], plaque forming units (pfu), etc. As used herein, the term “proliferative activity” referes to an activity that promotes cell proliferation and replication.

Administer/Administration: The terms “administration” and “administer” are used interchangeably herein to refer the act of contacting a subject, including contacting a cell, tissue, organ, or biological fluid of the subject in vitro, in vivo or ex vivo with an agent (e.g. an ortholog, an IL2 ortholog, an engineered cell expressing an orthogonal receptor, an engineered cell expressing an orthogonal IL2 receptor, a CAR-T cell expressing an orthogonal IL2 receptor, a chemotherapeutic agent, an antibody, or a pharmaceutical formulation comprising one or more of the foregoing). Administration of an agent may be achieved through any of a variety of art recognized methods including but not limited to the topical administration, intravascular injection (including intravenous or intraarterial infusion), intradermal injection, subcutaneous injection, intramuscular injection, intraperitoneal injection, inhalation and the like. The term “administration” includes contact of an agent to the cell, tissue or organ as well as the contact of an agent to a fluid, where the fluid is in contact with the cell, tissue or organ.

Affinity: As used herein the term “affinity” refers to the degree of specific binding of a first molecule (e.g., a ligand) to a second molecule (e.g., a receptor) and is measured by the equilibrium dissociation constant (K_{D}), a ratio of the dissociation rate constant between the molecule and its target (K_{off}) and the association rate constant between the molecule and its target (Kₒₙ).

Agonist: As used herein, the term “agonist” refers a first agent that specifically binds a second agent (“target”) and interacts with the target to cause or promote an increase in the activation of the target. In some instances, agonists are activators of receptor proteins that modulate cell activation, enhance activation, sensitize cells to activation by a second agent, or up-regulate the expression of one or more genes, proteins, ligands, receptors, biological pathways, that may result in cell proliferation or pathways that result in cell cycle arrest or cell death such as by apoptosis. In some embodiments, an agonist is an agent that binds to a receptor and alters the receptor state, resulting in a biological response. The response mimics the effect of the endogenous activator of the receptor. The term “agonist” includes partial agonists, full agonists and superagonists. An agonist may be described as a “full agonist” when such agonist which leads to a substantially full biological response (i.e., the response associated with the naturally occurring ligand/receptor binding interaction) induced by receptor under study, or a partial agonist. In contrast to agonists, antagonists may specifically bind to a receptor but do not result the signal cascade typically initiated by the receptor and may to modify the actions of an agonist at that receptor. Inverse agonists are agents that produce a pharmacological response that is opposite in direction to that of an agonist. A “superagonist” is a type of agonist that is capable of producing a maximal response greater than the endogenous agonist for the target receptor, and thus has an activity of more than 100% of the native ligand. A super agonist is typically a synthetic molecule that exhibits greater than 110%, alternatively greater than 120%, alternatively greater than 130%, alternatively greater than 140%, alternatively greater than 150%, alternatively greater than 160%, or alternatively greater than 170% of the response in an evaluable quantitative or qualitative parameter of the naturally occurring form of the molecule when evaluated at similar concentrations in a comparable assay.

Antagonist: As used herein, the term “antagonist” or “inhibitor” refers a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist. Inhibitors are molecules that decrease, block, prevent, delay activation, inactivate, desensitize, or down-regulate, e.g., a gene, protein, ligand, receptor, biological pathway, or cell.

Antibody: As used herein, the term “antibody” refers collectively to: (a) glycosylated and non-glycosylated immunoglobulins (including but not limited to mammalian immunoglobulin classes IgG1, IgG2, IgG3 and IgG4) that specifically binds to target molecule and (b) immunoglobulin derivatives including but not limited to IgG(1-4)deltaC_{H}2, F(ab′)₂, Fab, ScFv, V_{H}, V_{L}, tetrabodies, triabodies, diabodies, dsFv, F(ab′)₃, scFv-Fc and (scFv)₂ that competes with the immunoglobulin from which it was derived for binding to the target molecule. The term antibody is not restricted to immunoglobulins derived from any particular mammalian species and includes murine, human, equine, camelids, human antibodies. The term antibody includes so called “heavy chain antibodies” or “VHHs” or “Nanobodies®” as typically obtained from immunization of camelids (including camels, llamas and alpacas (see, e.g. Hamers-Casterman, et al. (1993) Nature 363:446-448). Antibodies having a given specificity may also be derived from non-mammalian sources such as VHHs obtained from immunization of cartilaginous fishes including, but not limited to, sharks. The term “antibody” encompasses antibodies isolatable from natural sources or from animals following immunization with an antigen and as well as engineered antibodies including monoclonal antibodies, bispecific antibodies, tri-specific, chimeric antibodies, humanized antibodies, human antibodies, CDR-grafted, veneered, or deimmunized (e.g., to remove T-cell epitopes) antibodies. The term ““human antibody” includes antibodies obtained from human beings as well as antibodies obtained from transgenic mammals comprising human immunoglobulin genes such that, upon stimulation with an antigen the transgenic animal produces antibodies comprising amino acid sequences characteristic of antibodies produced by human beings. The term antibody includes both the parent antibody and its derivatives such as affinity matured, veneered, CDR grafted (including CDR grafted VHHs), humanized, camelized (in the case of non-camel derived VHHs), or binding molecules comprising binding domains of antibodies (e.g., CDRs) in non-immunoglobulin scaffolds. The term “antibody” is not limited to any particular means of synthesis and includes naturally occurring antibodies isolatable from natural sources and as well as engineered antibodies molecules that are prepared by “recombinant” means including antibodies isolated from transgenic animals that are transgenic for human immunoglobulin genes or a hybridoma prepared therefrom, antibodies isolated from a host cell transformed with a nucleic acid construct that results in expression of an antibody, antibodies isolated from a combinatorial antibody library including phage display libraries or chemically synthesized (e.g., solid phase protein synthesis). In one embodiment, an “antibody” is a mammalian immunoglobulin. In some embodiments, the antibody is a “full length antibody” comprising variable and constant domains providing binding and effector functions. In most instances, a full-length antibody comprises two light chains and two heavy chains, each light chain comprising a variable region and a constant region. In some embodiments the term “full length antibody” is used to refer to conventional IgG immunoglobulin structures comprising two light chains and two heavy chains, each light chain comprising a variable region and a constant region providing binding and effector functions. The term antibody includes antibody conjugates comprising modifications to prolong duration of action such as fusion proteins or conjugation to polymers (e.g., PEGylated) as described in more detail below.

Binding molecule: As used herein, the term “binding molecule” refers to a bivalent molecule that can bind to the extracellular domain of two cell surface receptors. In some embodiments, a binding molecule specifically binds to two different receptors (or domains or subunits thereof) such that the receptors (or domains or subunits) are maintained in proximity to each other such that the receptors (or domains or subunits), including domains thereof (e.g., intracellular domains) interact with each other and result in downstream signaling.

CDR: As used herein, the term “CDR” or “complementarity determining region” is intended to mean the non-contiguous antigen combining sites found within the variable region of both heavy and light chain immunoglobulin polypeptides. CDRs have been described by Kabat et al., J. Biol. Chem. 252:6609-6616 (1977); Kabat, et al., U.S. Dept. of Health and Human Services publication entitled “Sequences of proteins of immunological interest” (1991) (also referred to herein as “Kabat 1991” or “Kabat”); by Chothia, et al. (1987) J. Mol. Biol. 196:901-917 (also referred to herein as “Chothia”); and MacCallum, et al. (1996) J. Mol. Biol. 262:732-745, where the definitions include overlapping or subsets of amino acid residues when compared against each other. Nevertheless, application of either definition to refer to a CDR of an antibody or grafted antibodies or variants thereof is intended to be within the scope of the term as defined and used herein. The term “Chothia Numbering” as used herein is recognized in the arts and refers to a system of numbering amino acid residues based on the location of the structural loop regions (Chothia et al. 1986, Science 233:755-758; Chothia & Lesk 1987, JMB 196:901-917; Chothia et al. 1992, JMB 227:799-817). For purposes of the present disclosure, unless otherwise specifically identified, the positioning of CDRs2 and 3 in the variable region of an antibody follows Kabat numbering or simply, “Kabat.” The positioning of CDR1 in the variable region of an antibody follows a hybrid of Kabat and Chothia numbering schemes.

Clonotype: As used herein, a clonotype refers to a collection of binding molecules that originate from the same B-cell progenitor cell. The term “clonotype” is used to refer to a collection of antigen binding molecules that belong to the same germline family, have the same CDR3 lengths, and have 70% or greater homology in CDR3 sequence.

Comparable: As used herein, the term “comparable” is used to describe the degree of difference in two measurements of an evaluable quantitative or qualitative parameter. For example, where a first measurement of an evaluable quantitative parameter and a second measurement of the evaluable parameter do not deviate beyond a range that the skilled artisan would recognize as not producing a statistically significant difference in effect between the two results in the circumstances, the two measurements would be considered “comparable.” In some instances, measurements may be considered “comparable” if one measurement deviates from another by less than 30%, alternatively by less than 25%, alternatively by less than 20%, alternatively by less than 15%, alternatively by less than 10%, alternatively by less than 7%, alternatively by less than 5%, alternatively by less than 4%, alternatively by less than 3%, alternatively by less than 2%, or by less than 1%. In particular embodiments, one measurement is comparable to a reference standard if it deviates by less than 15%, alternatively by less than 10%, or alternatively by less than 5% from the reference standard.

Conservative Amino Acid Substitution: As used herein, the term “conservative amino acid substitution” refers to an amino acid replacement that changes a given amino acid to a different amino acid with similar biochemical properties (e.g., charge, hydrophobicity, and size). For example, the amino acids in each of the following groups can be considered as conservative amino acids of each other: (1) hydrophobic amino acids: alanine, isoleucine, leucine, tryptophan, phenylalanine, valine, proline, and glycine; (2) polar amino acids: glutamine, asparagine, histidine, serine, threonine, tyrosine, methionine, and cysteine; (3) basic amino acids: lysine and arginine; and (4) acidic amino acids: aspartic acid and glutamic acid.

Derived From: As used herein in the term “derived from”, in the context of an amino acid sequence or polynucleotide sequence (e.g., an amino acid sequence “derived from”), is meant to indicate that the polypeptide or nucleic acid has a sequence that is based on that of a reference polypeptide or nucleic acid (e.g., a naturally occurring polypeptide or an encoding nucleic acid), and is not meant to be limiting as to the source or method in which the protein or nucleic acid is made. By way of example, the term “derived from” includes homologs or variants of reference amino acid or DNA sequences.

Effective Concentration (EC): As used herein, the terms “effective concentration” or its abbreviation “EC” are used interchangeably to refer to the concentration of an agent (e.g., an hIL2 mutein) in an amount sufficient to effect a change in a given parameter in a test system. The abbreviation “E” refers to the magnitude of a given biological effect observed in a test system when that test system is exposed to a test agent. When the magnitude of the response is expressed as a factor of the concentration (“C”) of the test agent, the abbreviation “EC” is used. In the context of biological systems, the term Emax refers to the maximal magnitude of a given biological effect observed in response to a saturating concentration of an activating test agent. When the abbreviation EC is provided with a subscript (e.g., EC₄₀, EC₅₀, etc.) the subscript refers to the percentage of the Emax of the biological observed at that concentration. For example, the concentration of a test agent sufficient to result in the induction of a measurable biological parameter in a test system that is 30% of the maximal level of such measurable biological parameter in response to such test agent, this is referred to as the “EC₃₀” of the test agent with respect to such biological parameter. Similarly, the term “EC₁₀₀” is used to denote the effective concentration of an agent that results the maximal (100%) response of a measurable parameter in response to such agent. Similarly, the term EC₅₀ (which is commonly used in the field of pharmacodynamics) refers to the concentration of an agent sufficient to results in the half-maximal (50%) change in the measurable parameter. The term “saturating concentration” refers to the maximum possible quantity of a test agent that can dissolve in a standard volume of a specific solvent (e.g., water) under standard conditions of temperature and pressure. In pharmacodynamics, a saturating concentration of a drug is typically used to denote the concentration sufficient of the drug such that all available receptors are occupied by the drug, and EC₅₀ is the drug concentration to give the half-maximal effect. The EC of a particular effective concentration of a test agent may be abbreviated with respect to the with respect to particular parameter and test system.

Enriched: As used herein in the term “enriched” refers to a sample that is non-naturally manipulated so that a species (e.g. a molecule or cell) of interest is present in: (a) a greater concentration (e.g., at least 3-fold greater, alternatively at least 5-fold greater, alternatively at least 10-fold greater, alternatively at least 50-fold greater, alternatively at least 100-fold greater, or alternatively at least 1000-fold greater) than the concentration of the species in the starting sample, such as a biological sample (e.g., a sample in which the molecule naturally occurs or in which it is present after administration); or (b) a concentration greater than the environment in which the molecule was made (e.g., a recombinantly modified bacterial or mammalian cell).

Extracellular Domain: As used herein the term “extracellular domain” or its abbreviation “ECD” refers to the portion of a cell surface protein (e.g. a cell surface receptor) which is outside of the plasma membrane of a cell. The term “ECD” may include the extra-cytoplasmic portion of a transmembrane protein or the extra-cytoplasmic portion of a cell surface (or membrane associated protein).

Identity: As used herein, the term “percent (%) sequence identity” or “substantially identical” used in the context of nucleic acids or polypeptides, refers to a sequence that has at least 50% sequence identity with a reference sequence. Alternatively, percent sequence identity can be any integer from 50% to 100%. In some embodiments, a sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined with BLAST using standard parameters, as described below. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned. Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)). The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁻⁵, and most preferably less than about 10⁻²⁰.

Intracellular Signaling: As used herein, the terms “intracellular signaling” and “downstream signaling” are used interchangeably to refer to the to the cellular signaling process that is caused by the interaction of the intracellular domains (ICDs) of two or more cell surface receptors that are in proximity of each other. In rececptor complexes via the JAK/STAT pathway, the association of the ICDS of the receptor subunits brings the JAK domains of the ICDs into proximit which initiates a phosphorylation cascade in which STAT molecules are phosphorylated and translocate to the nucleus associating with particular nucleic acid sequences resulting in the activation and expression of particular genes in the cell. The binding molecules of the present disclosure provide intraceullar signaling characteristic of the IL10 receptor receptor when activated by its natural cognate IL10. To measure downstream signaling activity, a number of methods are available. For example, in some embodiments, one can measure JAK/STAT signaling by the presence of phosphorylated receptors and/or phosphorylated STATs. In other embodiments, the expression of one or more downstream genes, whose expression levels can be affected by the level of downstream signalinging caused by the binding molecule, can also be measured.

In An Amount Sufficient to Effect a Response: As used herein the phrase “in an amount sufficient to cause a response” is used in reference to the amount of a test agent sufficient to provide a detectable change in the level of an indicator measured before (e.g., a baseline level) and after the application of a test agent to a test system. In some embodiments, the test system is a cell, tissue or organism. In some embodiments, the test system is an in vitro test system such as a fluorescent assay. In some embodiments, the test system is an in vivo system which involves the measurement of a change in the level of a parameter of a cell, tissue, or organism reflective of a biological function before and after the application of the test agent to the cell, tissue, or organism. In some embodiments, the indicator is reflective of biological function or state of development of a cell evaluated in an assay in response to the administration of a quantity of the test agent. In some embodiments, the test system involves the measurement of a change in the level an indicator of a cell, tissue, or organism reflective of a biological condition before and after the application of one or more test agents to the cell, tissue, or organism. The term “in an amount sufficient to effect a response” may be sufficient to be a therapeutically effective amount but may also be more or less than a therapeutically effective amount.

In Need of Treatment: The term “in need of treatment” as used herein refers to a judgment made by a physician or other caregiver with respect to a subject that the subject requires or will potentially benefit from treatment. This judgment is made based on a variety of factors that are in the realm of the physician's or caregiver's expertise.

Isolated: As used herein the term “isolated” is used in reference to a polypeptide of interest that, if naturally occurring, is in an environment different from that in which it can naturally occur. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was synthesized, for example isolated from a recombinant cell culture comprising cells engineered to express the polypeptide or by a solution resulting from solid phase synthetic means.

Kabat Numbering: The term “Kabat numbering” as used herein is recognized in the art and refers to a system of numbering amino acid residues which are more variable than other amino acid residues (e.g., hypervariable) in the heavy and light chain regions of immunoglobulins (Kabat, et al., (1971) Ann. NY Acad. Sci. 190:382-93; Kabat, et al., (1991) Sequences of Proteins of Immunological Interest, Fifth Edition, U.S. Department of Health and Human Services, NIH Publication No. 91-3242). The term “Chothia Numbering” as used herein is recognized in the arts and refers to a system of numbering amino acid residues based on the location of the structural loop regions (Chothia et al. 1986, Science 233:755-758; Chothia & Lesk 1987, JMB 196:901-917; Chothia et al. 1992, JMB 227:799-817). For purposes of the present disclosure, unless otherwise specifically identified, the positioning of CDRs 2 and 3 in the variable region of an antibody follows Kabat numbering or simply, “Kabat.” The positioning of CDR1 in the variable region of an antibody follows a hybrid of Kabat and Chothia numbering schemes.

Ligand: As used herein, the term “ligand” refers to a molecule that exhibits specific binding to a receptor and results in a change in the biological activity of the receptor so as to effect a change in the activity of the receptor to which it binds. In one embodiment, the term “ligand” refers to a molecule, or complex thereof, that can act as an agonist or antagonist of a receptor. As used herein, the term “ligand” encompasses natural and synthetic ligands. “Ligand” also encompasses small molecules, e.g., peptide mimetics of cytokines and peptide mimetics of antibodies. The complex of a ligand and receptor is termed a “ligand-receptor complex.”

As used herein, the term “linker” refers to a linkage between two elements, e.g., protein domains. A linker can be a covalent bond or a peptide linker. The term “bond” refers to a chemical bond, e.g., an amide bond or a disulfide bond, or any kind of bond created from a chemical reaction, e.g., chemical conjugation. The term “peptide linker” refers to an amino acid or polyeptide that may be employed to link two protein domains to provide space and/or flexibility between the two protein domains.

Modulate: As used herein, the terms “modulate”, “modulation” and the like refer to the ability of a test agent to cause a response, either positive or negative or directly or indirectly, in a system, including a biological system, or biochemical pathway. The term modulator includes both agonists (including partial agonists, full agonists and superagonists) and antagonists.

Multimerization: As used herein, the term “multimerization” refers to two or more cell surface receptors, or domains or subunits thereof, being brought in close proximity to each other such that the receptors, or domains or subunits thereof, can interact with each other and cause intracellular signaling.

N-Terminus: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. The terms “immediately N-terminal” or “immediately C-terminal” are used to refers to a position of a first amino acid residue relative to a second amino acid residue where the first and second amino acid residues are covalently bound to provide a contiguous amino acid sequence.

Neoplastic Disease: As used herein and as discussed in more detail below, the term “neoplastic disease” refers to disorders or conditions in a subject arising from cellular hyper-proliferation or unregulated (or dysregulated) cell replication. The term neoplastic disease refers to disorders arising from the presence of neoplasms in the subject. Neoplasms may be classified as: (1) benign (2) pre-malignant (or “pre-cancerous”); and (3) malignant (or “cancerous”). The term “neoplastic disease” includes neoplastic-related diseases, disorders and conditions referring to conditions that are associated, directly or indirectly, with neoplastic disease, and includes, e.g., angiogenesis and precancerous conditions such as dysplasia or smoldering multiple myeloma. Examples of benign disorders arising from dysregulated cell replication include hypertrophic scars such as keloid scars.

Nucleic Acid: The terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the

Operably Linked: The term “operably linked” is used herein to refer to the relationship between molecules, typically polypeptides or nucleic acids, which are arranged in a construct such that each of the functions of the component molecules is retained although the operable linkage may result in the modulation of the activity, either positively or negatively, of the individual components of the construct. For example, the operable linkage of a polyethylene glycol (PEG) molecule to a wild-type protein may result in a construct where the biological activity of the protein is diminished relative to the to the wild-type molecule, however the two are nevertheless considered operably linked. When the term “operably linked” is applied to the relationship of multiple nucleic acid sequences encoding differing functions, the multiple nucleic acid sequences when combined into a single nucleic acid molecule that, for example, when introduced into a cell using recombinant technology, provides a nucleic acid which is capable of effecting the transcription and/or translation of a particular nucleic acid sequence in a cell. For example, the nucleic acid sequence encoding a signal sequence may be considered operably linked to DNA encoding a polypeptide if it results in the expression of a preprotein whereby the signal sequence facilitates the secretion of the polypeptide; a promoter or enhancer is considered operably linked to a coding sequence if it affects the transcription of the sequence; or a ribosome binding site is considered operably linked to a coding sequence if it is positioned so as to facilitate translation. Generally in the context of nucleic acid molecules, the term “operably linked” means that the nucleic acid sequences being linked are contiguous, and, in the case of a secretory leader or associated subdomains of a molecule, contiguous and in reading phase. However, certain genetic elements such as enhancers may function at a distance and need not be contiguous with respect to the sequence to which they provide their effect but nevertheless may be considered operably linked.

Partial Agonist: As used herein, the term “partial agonist” refers to a molecule that specifically binds that bind to and activate a given receptor but possess only partial activation the receptor relative to a full agonist. Partial agonists may display both agonistic and antagonistic effects. For example, when both a full agonist and partial agonist are present, the partial agonist acts as a competitive antagonist by competing with the full agonist for the receptor binding resulting in net decrease in receptor activation relative to the contact of the receptor with the full agonist in the absence of the partial agonist. Clinically, partial agonists can be used to activate receptors to give a desired submaximal response when inadequate amounts of the endogenous ligand are present, or they can reduce the overstimulation of receptors when excess amounts of the endogenous ligand are present. The maximum response (Emax) produced by a partial agonist is called its intrinsic activity and may be expressed on a percentage scale where a full agonist produced a 100% response. A In some embodiments, the IL10R binding molecule has a reduced Emax compared to the Emax caused by IL10. Emax reflects the maximum response level in a cell type that can be obtained by a ligand (e.g., a binding molecule described herein or the native cytokine (e.g., IL10)). In some embodiments, the IL10R binding molecule described herein has at least 1% (e.g., between 1% and 100%, between 10% and 100%, between 20% and 100%, between 30% and 100%, between 40% and 100%, between 50% and 100%, between 60% and 100%, between 70% and 100%, between 80% and 100%, between 90% and 100%, between 10% and 90%, between 10% and 80%, between 1% and 70%, between 1% and 60%, between 1% and 50%, between 1% and 40%, between 1% and 30%, between 1% and 20%, or between 1% and 10%) of the Emax caused by IL10. In other embodiments, the Emax of the IL10R binding molecule described herein is greater (e.g., at least 1%, 5%, 10%, 15%, 20%, 25%, 30%, 35%, 40%, 45%, or 50% greater) than the Emax of the natural ligand, IL10. In some embodiments, by varying the linker length of the IL10R binding molecules, the Emax of the IL10R binding molecule can be changed. The IL10R binding molecule can cause Emax in the most desired cell types, and a reduced Emax in other cell types.

Polypeptide: As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The term polypeptide include fusion proteins, including, but not limited to, fusion proteins with a heterologous amino acid sequence; fusion proteins with heterologous and homologous leader sequences; fusion proteins with or without N-terminal methionine residues; fusion proteins with amino acid sequences that facilitate purification such as chelating peptides; fusion proteins with immunologically tagged proteins; fusion proteins comprising a peptide with immunologically active polypeptide fragment (e.g. antigenic diphtheria or tetanus toxin or toxoid fragments) and the like.

Prevent: As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed due to genetic, experiential or environmental factors to having a particular disease, disorder or condition. In certain instances, the terms “prevent”, “preventing”, “prevention” are also used to refer to the slowing of the progression of a disease, disorder or condition from a present its state to a more deleterious state.

Proximity: As used herein, the term “proximity” refers to the spatial proximity or physical distance between two cell surface receptors, or domains or subunits thereof, after a binding molecule described herein binds to the two cell surface receptors, or domains or subunits thereof. In some embodiments, after the binding molecule binds to the cell surface receptors, or domains or subunits thereof, the spatial proximity between the cell surface receptors, or domains or subunits thereof, can be, e.g., less than about 500 angstroms, such as e.g., a distance of about 5 angstroms to about 500 angstroms. In some embodiments, the spatial proximity amounts to less than about 5 angstroms, less than about 20 angstroms, less than about 50 angstroms, less than about 75 angstroms, less than about 100 angstroms, less than about 150 angstroms, less than about 250 angstroms, less than about 300 angstroms, less than about 350 angstroms, less than about 400 angstroms, less than about 450 angstroms, or less than about 500 angstroms. In some embodiments, the spatial proximity amounts to less than about 100 angstroms. In some embodiments, the spatial proximity amounts to less than about 50 angstroms. In some embodiments, the spatial proximity amounts to less than about 20 angstroms. In some embodiments, the spatial proximity amounts to less than about 10 angstroms. In some embodiments, the spatial proximity ranges from about 10 to 100 angstroms, from about 50 to 150 angstroms, from about 100 to 200 angstroms, from about 150 to 250 angstroms, from about 200 to 300 angstroms, from about 250 to 350 angstroms, from about 300 to 400 angstroms, from about 350 to 450 angstroms, or about 400 to 500 angstroms. In some embodiments, the spatial proximity amounts to less than about 250 angstroms, alternatively less than about 200 angstroms, alternatively less than about 150 angstroms, alternatively less than about 120 angstroms, alternatively less than about 100 angstroms, alternatively less than about 80 angstroms, alternatively less than about 70 angstroms, or alternatively less than about 50 angstroms.

Receptor: As used herein, the term “receptor” refers to a polypeptide having a domain that specifically binds a ligand that binding of the ligand results in a change to at least one biological property of the polypeptide. In some embodiments, the receptor is a “soluble” receptor that is not associated with a cell surface. In some embodiments, the receptor is a cell surface receptor that comprises an extracellular domain (ECD) and a membrane associated domain which serves to anchor the ECD to the cell surface. In some embodiments of cell surface receptors, the receptor is a membrane spanning polypeptide comprising an intracellular domain (ICD) and extracellular domain (ECD) linked by a membrane spanning domain typically referred to as a transmembrane domain (TM). The binding of the ligand to the receptor results in a conformational change in the receptor resulting in a measurable biological effect. In some instances, where the receptor is a membrane spanning polypeptide comprising an ECD, TM and ICD, the binding of the ligand to the ECD results in a measurable intracellular biological effect mediated by one or more domains of the ICD in response to the binding of the ligand to the ECD. In some embodiments, a receptor is a component of a multi-component complex to facilitate intracellular signaling. For example, the ligand may bind a cell surface molecule having not associated with any intracellular signaling alone but upon ligand binding facilitates the formation of a multimeric complex that results in intracellular signaling.

Recombinant: As used herein, the term “recombinant” is used as an adjective to refer to the method by a polypeptide, nucleic acid, or cell that was modified using recombinant DNA technology. A recombinant protein is a protein produced using recombinant DNA technology and may be designated as such using the abbreviation of a lower case “r” (e.g., rhIL2) to denote the method by which the protein was produced. Similarly, a cell is referred to as a “recombinant cell” if the cell has been modified by the incorporation (e.g., transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. The techniques and protocols for recombinant DNA technology are well known in the art such as those can be found in Sambrook, et al. (1989) Molecular Cloning: A Laboratory Manual (2d ed., Cold Spring Harbor Laboratory Press, Plainview, N.Y.) and other standard molecular biology laboratory manuals.

Response: The term “response,” for example, of a cell, tissue, organ, or organism, encompasses a quantitative or qualitative change in a evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation) where the change is correlated with the activation, stimulation, or treatment, with or contact with exogenous agents or internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects. A “response” may be evaluated in vitro such as through the use of assay systems, surface plasmon resonance, enzymatic activity, mass spectroscopy, amino acid or protein sequencing technologies. A “response” may be evaluated in vivo quantitatively by evaluation of objective physiological parameters such as body temperature, bodyweight, tumor volume, blood pressure, results of X-ray or other imaging technology or qualitatively through changes in reported subjective feelings of well-being, depression, agitation, or pain. In some embodiments, the level of proliferation of CD3 activated primary human T-cells may be evaluated in a bioluminescent assay that generates a luminescent signal that is proportional to the amount of ATP present which is directly proportional to the number of viable cells present in culture as described in Crouch, et al. (1993) J. Immunol. Methods 160: 81-8 or using commercially available assays such as the CellTiter-Glo® 2.0 Cell Viability Assay or CellTiter-Glo® 3D Cell Viability kits commercially available from Promega Corporation, Madison WI 53711 as catalog numbers G9241 and G9681 in substantial accordance with the instructions provided by the manufacturer. In some embodiments, the level of activation of T cells in response to the administration of a test agent may be determined by flow cytometric methods as described as determined by the level of STAT (e.g., STAT1, STAT3, STAT5) phosphorylation in accordance with methods well known in the art.

Significantly Reduced Binding: As used herein, the term “exhibits significantly reduced binding” is used with respect to a variant of a first molecule (e.g. a ligand) which exhibits a significant reduction in the affinity for a second molecule (e.g. receptor) relative to the parent form of the first molecule. With respect to antibody variants, an antibody variant “exhibits significantly reduced binding” if the variant binds to the native form of the receptor with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent antibody from which the variant was derived. Similarly, with respect to variant ligands, a variant ligand “exhibits significantly reduced binding” if the variant ligand binds to a receptor with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent ligand from which the variant ligand was derived. Similarly, with respect to variant receptors, a variant ligand “exhibits significantly reduced binding” if the affinity of the variant receptors binds to a with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent receptor from which the variant receptor was derived.

Single Domain Antibody (sdAb): The term “single-domain antibody” or “sdAbs,” refers to an antibody having a single (only one) monomeric variable antibody domain. A sdAb is able to bind selectively to a specific antigen. A VHH is an example of a sdAb.

Specifically Binds: As used herein the term “specifically binds” refers to the degree of affinity for which a first molecule exhibits with respect to a second molecule. In the context of binding pairs (e.g., ligand/receptor, antibody/antigen) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample. In a particular embodiment, where the first molecule of the binding pair is an antibody, the antibody specifically binds to the antigen (or antigenic determinant (epitope) of a protein, antigen, ligand, or receptor) if the equilibrium dissociation constant (Ku) between antibody and the antigen is lesser than about 10⁻⁶ M, alternatively lesser than about 10⁻⁸ M, alternatively lesser than about 10⁻¹⁰ M, alternatively lesser than about 10⁻¹¹ M, lesser than about 10⁻¹² M as determined by, e.g., Scatchard analysis (Munsen, et al. (1980) Analyt. Biochem. 107:220-239). In one embodiment, a ligand specifically binds to a receptor if the dissociation is greater than about 10⁵M, alternatively greater than about 10⁶ M, alternatively greater than about 10⁷M, alternatively greater than about 10⁸M, alternatively greater than about 109 M, alternatively greater than about 10¹⁰ M, or alternatively greater than about 10¹¹ M. Specific binding may be assessed using techniques known in the art including but not limited to competition ELISA assays, radioactive ligand binding assays (e.g., saturation binding, Scatchard plot, nonlinear curve fitting programs and competition binding assays); non-radioactive ligand binding assays (e.g., fluorescence polarization (FP), fluorescence resonance energy transfer (FRET); liquid phase ligand binding assays (e.g., real-time polymerase chain reaction (RT-qPCR), and immunoprecipitation); and solid phase ligand binding assays (e.g., multiwell plate assays, on-bead ligand binding assays, on-column ligand binding assays, and filter assays)) and surface plasmon resonance assays (see, e.g., Drescher et al., (2009) Methods Mol Biol 493:323-343 with commercially available instrumentation such as the Biacore 8+, Biacore S200, Biacore T200 (GE Healthcare Bio-Sciences, 100 Results Way, Marlborough MA 01752). In some embodiments, the present disclosure provides molecules (e.g., IL12RB1 binding sdAbs) that specifically bind to the hIL12RB1 isoform. As used herein, the binding affinity of an IL12RB1 binding molecule for the IL12RB1, the binding affinity may be determined and/or quantified by surface plasmon resonance (“SPR”). In evaluating binding affinity of an IL12RB1 binding molecule for the IL12RB1, either member of the binding pair may be immobilized, and the other element of the binding pair be provided in the mobile phase. In some embodiments, the sensor chip on which the protein of interest is to be immobilized is conjugated with a substance to facilitate binding of the protein of interest such as nitrilotriacetic acid (NTA) derivatized surface plasmon resonance sensor chips (e.g., Sensor Chip NTA available from Cytiva Global Life Science Solutions USA LLC, Marlborough MA as catalog number BR100407), as anti-His tag antibodies (e.g. anti-histidine CM5 chips commercially available from Cytiva, Marlborough MA), protein A or biotin. Consequently, to evaluate binding, it is frequently necessary to modify the protein to provide for binding to the substance conjugated to the surface of the chip. For example, the one member of the binding pair to be evaluated by incorporation of a chelating peptide comprising poly-histidine sequence (e.g., 6×His (SEQ ID NO: 532) or 8×His (SEQ ID NO: 534)) for retention on a chip conjugated with NTA. In some embodiments, the binding molecule may be immobilized on the chip and receptor subunit (or ECD fragment thereof) be provided in the mobile phase. Alternatively, the receptor subunit (or ECD fragment thereof) may be immobilized on the chip and the binding molecule be provided in the mobile phase. In either circumstance, it should be noted that modifications of some proteins for immobilization on a coated SPR chip may interfere with the binding properties of one or both components of the binding pair to be evaluated by SPR. In such cases, it may be necessary to switch the mobile and bound elements of the binding pair or use a chip with a binding agent that facilitates non-interfering conjugation of the protein to be evaluated. Alternatively, when evaluating the binding affinity of binding molecule for receptor subunit using SPR, the binding molecule may be derivatized by the C-terminal addition of a poly-His sequence (e.g., 6×His (SEQ ID NO: 532) or 8×His (SEQ ID NO: 534)) and immobilized on the NTA derivatized sensor chip and the receptor subunit for which the binding affinity is being evaluated is provided in the mobile phase. The means for incorporation of a poly-His sequence into the C-terminus of the binding molecule produced by recombinant DNA technology is well known to those of skill in the relevant art of biotechnology.

Subject: The terms “recipient”, “individual”, “subject”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

Substantially Pure: As used herein, the term “substantially pure” indicates that a component of a composition makes up greater than about 50%, alternatively greater than about 60%, alternatively greater than about 70%, alternatively greater than about 80%, alternatively greater than about 90%, alternatively greater than about 95%, of the total content of the composition. A protein that is “substantially pure” comprises greater than about 50%, alternatively greater than about 60%, alternatively greater than about 70%, alternatively greater than about 80%, alternatively greater than about 90%, alternatively greater than about 95%, of the total content of the composition.

Suffering From: As used herein, the term “suffering from” refers to a determination made by a physician with respect to a subject based on the available information accepted in the field for the identification of a disease, disorder or condition including but not limited to X-ray, CT-scans, conventional laboratory diagnostic tests (e.g., blood count), genomic data, protein expression data, immunohistochemistry, that the subject requires or will benefit from treatment. The term suffering from is typically used in conjunction with a particular disease state such as “suffering from a neoplastic disease” refers to a subject which has been diagnosed with the presence of a neoplasm.

T-cell: As used herein the term “T-cell” or “T cell” is used in its conventional sense to refer to a lymphocyte that differentiates in the thymus, possess specific cell-surface antigen receptors, and include some that control the initiation or suppression of cell-mediated and humoral immunity and others that lyse antigen-bearing cells. In some embodiments the T cell includes without limitation naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, Tregs, inducible Tregs; memory T cells, e.g. central memory T cells, effector memory T cells, NKT cells, tumor infiltrating lymphocytes (TILs) and engineered variants of such T-cells including but not limited to CAR-T cells, recombinantly modified TILs and TCR engineered cells.

Terminus/Terminal: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” refers to the position of a first amino acid residue relative to a second amino acid residue in a contiguous polypeptide sequence, the first amino acid being closer to the N-terminus of the polypeptide. “Immediately C-terminal” refers to the position of a first amino acid residue relative to a second amino acid residue in a contiguous polypeptide sequence, the first amino acid being closer to the C-terminus of the polypeptide.

Therapeutically Effective Amount: As used herein, the term The phrase “therapeutically effective amount” is used in reference to the administration of an agent to a subject, either alone or as part of a pharmaceutical composition or treatment regimen, in a single dose or as part of a series of doses in an amount capable of having any detectable, positive effect on any symptom, aspect, or characteristic of a disease, disorder or condition when administered to the subject. The therapeutically effective amount can be ascertained by measuring relevant physiological effects, and it may be adjusted in connection with a dosing regimen and in response to diagnostic analysis of the subject's condition, and the like. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, modification of biomarker levels, increase in duration of survival, extended duration of progression free survival, extension of the time to progression, increased time to treatment failure, extended duration of event free survival, extension of time to next treatment, improvement objective response rate, improvement in the duration of response, and the like that that are relied upon by clinicians in the field for the assessment of an improvement in the condition of the subject in response to administration of an agent.

Treg Cell or Regulatory T Cell. The terms “regulatory T cell” or “Treg cell” as used herein refers to a type of CD4⁺ T cell that can suppress the responses of other T cells including but not limited to effector T cells (Teff). Treg cells are characterized by expression of CD4, the a-subunit of the IL2 receptor (CD25), and the transcription factor forkhead box P3 (FOXP3) (Sakaguchi, Annu Rev Immunol 22, 531-62 (2004). By “conventional CD4⁺ T cells” is meant CD4⁺ T cells other than regulatory T cells.

Transmembrane Domain: The term “transmembrane domain” or “TM” refers to the domain of a membrane spanning polypeptide which, when the membrane spanning polypeptide is associated with a cell membrane, is which is embedded in the cell membrane and is in peptidyl linkage with the extracellular domain (ECD) and the intracellular domain (ICD) of a membrane spanning polypeptide. A transmembrane domain may be homologous (naturally associated with) or heterologous (not naturally associated with) with either or both of the extracellular and/or intracellular domains. A transmembrane domain may be homologous (naturally associated with) or heterologous (not naturally associated with) with either or both of the extracellular and/or intracellular domains. In some embodiments, where the receptor is chimeric receptor comprising the intracellular domain derived from a first parental receptor and a second extracellular domains are derived from a second different parental receptor, the transmembrane domain of the chimeric receptor is the transmembrane domain normally associated with either the ICD or the ECD of the parent receptor from which the chimeric receptor is derived. Alternatively, the transmembrane domain of the receptor may be an artificial amino acid sequence which spans the plasma membrane. In some embodiments, where the receptor is chimeric receptor comprising the intracellular domain derived from a first parental receptor and a second extracellular domains are derived from a second different parental receptor, the transmembrane domain of the chimeric receptor is the transmembrane domain normally associated with either the ICD or the ECD of the parent receptor from which the chimeric receptor is derived.

Treat: The terms “treat”, “treating”, treatment” and the like refer to a course of action (such as administering a binding molecule described herein, or a pharmaceutical composition comprising same) initiated with respect to a subject after a disease, disorder or condition, or a symptom thereof, has been diagnosed, observed, or the like in the subject so as to eliminate, reduce, suppress, mitigate, or ameliorate, either temporarily or permanently, at least one of the underlying causes of such disease, disorder, or condition afflicting a subject, or at least one of the symptoms associated with such disease, disorder, or condition. The treatment includes a course of action taken with respect to a subject suffering from a disease where the course of action results in the inhibition (e.g., arrests the development of the disease, disorder or condition or ameliorates one or more symptoms associated therewith) of the disease in the subject.

VHH: As used herein, the term “VHH” is a type of sdAb that has a single monomeric heavy chain variable antibody domain. Such antibodies can be found in or produced from Camelid mammals (e.g., camels, llamas) which are naturally devoid of light chains VHHs can be obtained from immunization of camelids (including camels, llamas, and alpacas (see, e.g., Hamers-Casterman, et al. (1993) Nature 363:446-448) or by screening libraries (e.g., phage libraries) constructed in VHH frameworks. Antibodies having a given specificity may also be derived from non-mammalian sources such as VHHs obtained from immunization of cartilaginous fishes including, but not limited to, sharks. In a particular embodiment, a VHH in a bispecific VHH² binding molecule described herein binds to a receptor (e.g., the first receptor or the second receptor of the natural or non-natural receptor pairs) if the equilibrium dissociation constant between the VHH and the receptor is greater than about 10⁻⁶ M, alternatively lesser than about 10⁻⁸ M, alternatively lesser than about 10⁻¹⁰ M, alternatively lesser than about 10⁻¹¹ M, alternatively lesser than about 10⁻¹⁰ M, lesser than about 10⁻¹² M as determined by, e.g., Scatchard analysis (Munsen, et al. 1980 Analyt. Biochem. 107:220-239). Standardized protocols for the generation of single domain antibodies from camelids are well known in the scientific literature. See, e.g., Vincke, et al (2012) Chapter 8 in Methods in Molecular Biology, Walker, J. editor (Humana Press, Totowa NJ). Specific binding may be assessed using techniques known in the art including but not limited to competition ELISA, BIACORE® assays and/or KINEXA® assays. In some embodiments, a VHH described herein can be humanized to contain human framework regions. Examples of human germlines that could be used to create humanized VHHs include, but are not limited to, VH3-23 (e.g., UniProt ID: P01764), VH3-74 (e.g., UniProt ID: A0A0B4J1X5), VH3-66 (e.g., UniProt ID: A0A0C4DH42), VH3-30 (e.g., UniProt ID: P01768), VH3-11 (e.g., UniProt ID: P01762), and VH3-9 (e.g., UniProt ID: P01782).

VHH²: As used herein, the term “VHH²” and “bispecific VHH²” and “VHH dimer” refers to are used interchangeably to refer to a subtype of the binding molecules of the present disclosure wherein the first and second sdAbs are both VHHs and first VHH binding to a first receptor, or domain or subunit thereof, and a second VHH binding to a second receptor, or domain or subunit thereof.

Wild Type: As used herein, the term “wild type” or “WT” or “native” is used to refer to an amino acid sequence or a nucleotide sequence that is found in nature and that has not been altered by the hand of man.

### Interleukin 10 Receptor Binding Molecules

The present invention provides IL10R binding molecules that are synthetic ligands of the IL10 receptor.

The IL10R binding molecules of the present disclosure comprise two or more single domain antibodies that selectively bind to the extracellular domain of the IL10Ra and IL10Rb receptor subunits. In one embodiment, the present disclosure provides an IL10 receptor (IL10R) binding molecule that is a ligand for the IL10R receptor, the IL10R receptor binding molecule comprising:
(e) a first single domain antibody (sdAb) that specifically binds to the extracellular domain the IL10Ra subunit of the IL10 receptor (an “IL10Ra sdAb”), and
(f) a second single domain antibody that specifically binds to the extracellular domain of the IL10Rb subunit of the IL10 receptor (an “IL10Rb sdAb”),
   wherein:
the first sdAb and second sdAb are in stable association;
the IL10Ra and IL10Rb subunits of the IL10 receptor are dimerized in response to contact with the IL10R binding molecule; and
contacting a cell expressing the IL10Ra and IL10Rb with an effective amount of the IL10R binding molecule results in the intracellular domains of IL10Ra and IL10Rb being brought into proximity and intracellular signaling.

The IL10R binding molecules of the present disclosure are useful in the treatment or prevention of disease in mammalian subjects, In some embodiments, the present disclosure provides for the treatment or prevention of autoimmune disease in a mammalian subject by the administration of a therapeutically effective amount of an IL10R binding molecule of the present disclosure. In some embodiments, the present disclosure provides for the treatment or prevention of infectious disease, including viral and chronic viral infections, in a mammalian subject by the administration of a therapeutically effective amount of an IL10R binding molecule of the present disclosure. In some embodiments, the present disclosure provides for the treatment or prevention of neoplastic disease in a mammalian subject by the administration of a therapeutically effective amount of an IL10R binding molecule of the present disclosure. In some embodiments, the present disclosure provides for the treatment or prevention of neoplastic, infectious or autoimmune in a mammalian subject by the administration of a therapeutically effective amount of an IL10R binding molecule of the present disclosure in combination with one or more supplementary therapeutic agents.

In some embodiments, the present disclosure provides an IL10R binding molecule modified to provide prolonged duration of action in vivo in a mammalian subject and pharmaceutically acceptable formulations thereof.

The present disclosure further provides a pharmaceutically acceptable formulation of an IL10R binding molecule for the administration to a mammalian subject. The present disclosure further provides a pharmaceutically acceptable composition for administration to a mammalian subject the composition comprising a nucleic acid sequence encoding a polypeptide IL10R binding molecule, a recombinant viral or non-viral vector encoding or polypeptide IL10R binding molecule, or a recombinantly modified mammalian cell comprising a nucleic acid sequence encoding a polypeptide IL10R binding molecule, in each case the nucleic acid sequence operably linked to one or more expression control elements functional in a mammalian cell.

In some embodiments, the IL10R binding molecules of the present disclosure are polypeptides The present disclosure provides nucleic acid sequences enboding polypeptide IL10R binding molecules. The present disclosure further provides a recombinant vector comprising a nucleic acid sequences enboding polypeptide IL10R binding molecules. The present disclosure further provides a recombinantly modified mammalian cell comprising a nucleic acid enboding a polypeptide IL10R binding molecule. The present disclosure further provides methods for the recombinant production, isolation, purification and characterization of a polypeptide IL10R binding molecule. of recombinant vectors comprising a provides nucleic acid sequences enboding polypeptide IL10R binding molecules.

### IL10:

The cognate ligand of the IL10 receptor (IL10R) is the cytokine IL10. The term IL10 includes human and murine (or mouse) IL10. Human IL10 (hIL10) is non-covalently linked homodimeric protein comprising two identical subunits. Each human IL10 monomer is expressed as a 178 amino acid pre-protein comprising 18 amino acid signal sequence which is post-translationally removed to render a 160 amino acid mature protein. The canonical amino acid sequence of the mature IL-10 protein (UniProt Reference No. P22301) without the signal sequence (corresponding to amino acids 19-178 of the pre-protein) is:

(SEQ ID NO: 460)
SPGQGTQSENSCTHFPGNLPNMLRDLRDAFSRVKTFFQMKDQLDNLLL
KESLLEDFKGYLGCQALSEMIQFYLEEVMPQAENQDPDIKAHVNSLGE
NLKTLRLRLRRCHRFLPCENKSKAVEQVKNAFNKLQEKGIYKAMSEFD
IFINYIEAYMTMKIRN

Mouse (or murine) IL10 (mIL10) is a non-covalently linked homodimeric protein comprising two identical subunits. Each murine IL10 monomer is expressed as a 178 amino acid pre-protein comprising 18 amino acid signal sequence which is post-translationally removed to render a 160 amino acid mature protein. The canonical amino acid sequence of the mature IL-10 protein (UniProt Reference No. P18893) without the signal sequence (corresponding to amino acids 19-178 of the pre-protein) is:

(SEQ ID NO: 461)
SRGQYSREDNNCTHFPVGQSHMLLELRTAFSQVKTFFQTKDQLDNILL
TDSLMQDFKGYLGCQALSEMIQFYLVEVMPQAEKHGPEIKEHLNSLGE
KLKTLRMRLRRCHRFLPCENKSKAVEQVKSDFNKLQDQGVYKAMNEFD
IFINCIEAYMMIKMKS.

IL10R binding molecules activate IL10 signaling in a cell expressing the IL10 receptor. The present disclosure provides IL10R binding molecules engineered to provide selective levels of intracellular signalling in cells expressing the IL10 receptor. The present invention provides IL10R binding molecules engineered to generate intracellular signalling in particular cell types.

### IL10 Receptor

The IL10 receptor is a heterodimeric protein complex comprising the IL10Ra and IL10Rb subunits. The interaction of the IL10 on the surface of a mammlain cell expressing the IL10Ra and IL10Rb subunits results in dimerization of IL10Ra and IL10Rb and intracellular signaling. The intracellular signaling characteristic of IL10 mediated dimerization of the IL10Ra and IL10Rb is the activation of the JAK/STAT pathway, in particular the phosphorylation of STAT3 molecule which is a component of the intracellular signaling pathway that, in combination with other components of the signaling pathway results in modulation of gene expression. In some embodiments, the IL10 receptor the human IL10 receptor and the IL10 is the human IL10. In some embodiments the IL10 receptor is the murine IL10 receptor and the IL10 is the murine IL10. As used herein, the terms “IL10 receptor receptor” and “IL10 receptor” and “IL10R” are used interchangeably to refer to a heterodimeric complex comprising IL10Ra and IL10Rb. The term IL10R includes IL10 receptors of any mammal including but not limited to human beings, dogs, cats, mice, monkeys, cows, and pigs.

The IL10Ra component of the human IL10 receptor is the human IL10Ra (hIL10Ra) protein. The canonical full length hIL10Ra protein is a polypeptide possessing the amino acid sequence:

(SEQ ID NO: 452)
MLPCLVVLLAALLSLRLGSDAHGTELPSPPSVWFEAEFFHHILHWTPI
PNQSESTCYEVALLRYGIESWNSISNCSQTLSYDLTAVTLDLYHSNGY
RARVRAVDGSRHSNWTVTNTRFSVDEVTLTVGSVNLEIHNGFILGKIQ
LPRPKMAPANDTYESIFSHFREYEIAIRKVPGNFTFTHKKVKHENFSL
LTSGEVGEFCVQVKPSVASRSNKGMWSKEECISLTRQYFTVTNVIIFF
AFVLLLSGALAYCLALQLYVRRRKKLPSVLLFKKPSPFIFISQRPSPE
TQDTIHPLDEEAFLKVSPELKNLDLHGSTDSGFGSTKPSLQTEEPQFL
LPDPHPQADRTLGNREPPVLGDSCSSGSSNSTDSGICLQEPSLSPSTG
PTWEQQVGSNSRGQDDSGIDLVQNSEGRAGDTQGGSALGHHSPPEPEV
PGEEDPAAVAFQGYLRQTRCAEEKATKTGCLEEESPLTDGLGPKFGRC
LVDEAGLHPPALAKGYLKQDPLEMTLASSGAPTGQWNQPTEEWSLLAL
SSCSDLGISDWSFAHDLAPLGCVAAPGGLLGSFNSDLVTLPLISSLQS
SE

For purposes of the present disclosure, the numbering of amino acid residues of the hIL10Ra polypeptides as described herein is made in accordance with the numbering of this canonical sequence UniProg Database Reference No. Q13651. Amino acids 1-21 of SEQ ID NO:452 are identified as the signal peptide of the IL10Ra, amino acids 22-235 of SEQ ID NO:452 are identified as the extracellular domain, amino acids 236-256 of SEQ ID NO:452 are identified as the transmembrane domain, and amino acids 257-578 of SEQ ID NO: 452 are identified as the intracellular domain.

The IL10Ra component of the mouse IL10 receptor is the mouse IL10Ra (mIL10Ra) protein. mIL10Ra is expressed as 575 amino acid pre-protein containing a 16 amino acid signal sequence which is post-translationally cleaved to render a 569 amino acid mature protein The canonical full length mIL10Ra is a polypeptide having the amino acid sequence:

(SEQ ID NO: 454)
MLSRLLPFLVTISSLSLEFIAYGTELPSPSYVWFEARFFQHILHWKPI
PNQSESTYYEVALKQYGNSTWNDIHICRKAQALSCDLTTFTLDLYHRS
YGYRARVRAVDNSQYSNWTTTETRFTVDEVILTVDSVTLKAMDGIIYG
TIHPPRPTITPAGDEYEQVFKDLRVYKISIRKFSELKNATKRVKQETF
TLTVPIGVRKFCVKVLPRLESRINKAEWSEEQCLLITTEQYFTVTNLS
ILVISMLLFCGILVCLVLQWYIRHPGKLPTVLVFKKPHDFFPANPLCP
ETPDAIHIVDLEVFPKVSLELRDSVLHGSTDSGFGSGKPSLQTEESQF
LLPGSHPQIQGTLGKEESPGLQATCGDNTDSGICLQEPGLHSSMGPAW
KQQLGYTHQDQDDSDVNLVQNSPGQPKYTQDASALGHVCLLEPKAPEE
KDQVMVTFQGYQKQTRWKAEAAGPAECLDEEIPLTDAFDPELGVHLQD
DLAWPPPALAAGYLKQESQGMASAPPGTPSRQWNQLTEEWSLLGVVSC
EDLSIESWRFAHKLDPLDCGAAPGGLLDSLGSNLVTLPLISSLQVEE

For purposes of the present disclosure, the numbering of amino acid residues of the mouse IL10Ra polypeptides as described herein is made in accordance with the numbering of this canonical sequence UniProt Database Reference No. Q61727. Amino acids 1-16 of SEQ ID NO: 454 are identified as the signal peptide of the mIL10Ra, amino acids 17-241 of SEQ ID NO: 454 are identified as the extracellular domain, amino acids 242-262 of SEQ ID NO: 454 are identified as the transmembrane domain, and amino acids 263-575 of SEQ ID NO: 454 are identified as the intracellular domain.

hIL10Rb is expressed as a 325 amino acid pre-protein, the first 19 amino acids comprising a signal sequence which is post-translationally cleaved in the mature 306 amino acid protein. Amino acids 20-220 (amino acids 1-201 of the mature protein) correspond to the extracellular domain, amino acids 221-242 (amino acids 202-223 of the mature protein) correspond to the 22 amino acid transmembrane domain, and amino acids 243-325 (amino acids 224-306 of the mature protein) correspond to the intracellular domain The canonical full length hIL10Rb precursor is a polypeptide having the amino acid sequence:

(SEQ ID NO: 456)
MAWSLGSWLGGCLLVSALGMVPPPENVRMNSVNFKNILQWESPAFAKGN
LTFTAQYLSYRIFQDKCMNTTLTECDFSSLSKYGDHTLRVRAEFADEHS
DWVNITFCPVDDTIIGPPGMQVEVLADSLHMRFLAPKIENEYETWTMKN
VYNSWTYNVQYWKNGTDEKFQITPQYDFEVLRNLEPWTTYCVQVRGFLP
DRNKAGEWSEPVCEQTTHDETVPSWMVAVILMASVFMVCLALLGCFALL
WCVYKKTKYAFSPRNSLPQHLKEFLGHPHHNTLLFFSFPLSDENDVFDK
LSVIAEDSESGKQNPGDSCSLGTPPGQGPQS

For purposes of the present disclosure, the numbering of amino acid residues of the human IL10Rb polypeptides as described herein is made in accordance with the numbering of the canonical sequence (UniProt ID: Q08334, SEQ ID NO:456).

Murine IL10Rb (mIL10Rb) is expressed as a 349 amino acid pre-protein comprising a 19 amino acid N-terminal signal sequence. Amino acids 20-220 (amino acids 1-201 of the mature protein) correspond to the extracellular domain, amino acids 221-241 (amino acids 202-222 of the mature protein) correspond to the 21 amino acid transmembrane domain, and amino acids 242-349 (amino acids 223-330 of the mature protein) correspond to the intracellular domain. The canonical full length mIL10Rb precursor protein including the signal sequence is a polypeptide of is referenced at UniProtKB database as entry Q61190. The canonical full length mIL10Rb precursor protein including the signal sequence is a polypeptide of the amino acid sequence:

(SEQ ID NO: 458)
MAPCVAGWLGGFLLVPALGIPPPEKVRMNSVNFKNILQWEVPAFPKTNL
TFTAQYESYRSFQDHCKRTASTQCDFSHLSKYGDYTVRVRAELADEHSE
WVNVTFCPVEDTIIGPPEMQIESLAESLHLRFSAPQIENEPETWTLKNI
YDSWAYRVQYWKNGTNEKFQVVSPYDSEVLRNLEPWTTYCIQVQGFLLD
QNRTGEWSEPICERTGNDEITPSWIVAIILIVSVLVVFLFLLGCFVVLW
LIYKKTKHTFRSGTSLPQHLKEFLGHPHHSTFLLFSFPPPEEAEVFDKL
SIISEESEGSKQSPEDNCASEPPSDPGPRELESKDEAPSPPHDDPKLLT
STSEV

For purposes of the present disclosure, the numbering of amino acid residues of the murine IL10Rb polypeptides as described herein is made in accordance with the numbering of this canonical sequence (UniProt ID: Q61190, SEQ ID NO 458.

### Single Domain Antibody

The IL10R binding molecules of the present invention comprise two or more single domain antibodies. The term “single domain antibody” (sdAb) as used herein refers an antibody fragment consisting of a monomeric variable antibody domain that is able to bind specifically to an antigen and compete for binding with the parent antibody from which it is derived. The term “single domain antibody” includes scFv and VHH molecules. In some embodiments, one or both of the sdAbs of the cytokine receptor binding molecule is a an scFv. In some embodiments, one or both of the sdAbs is a VHH. In some embodiments, one or both of the sdAbs is a scFv.

The term single domain antibody includes engineered sdAbs including but not limited to chimeric sdAbs, CDR grafted sdAbs and humanized sdAbs. In some embodiments, the one or more of the sdAbs for incorporation into the IL10R binding molecules of the present disclosure are CDR grafted. CDRs obtained from antibodies, heavy chain antibodies, and sdAbs derived therefrom may be grafted onto alternative frameworks as described in Saerens, et al. (2005) J. Mol Biol 352:597-607 to generate CDR-grafted sdAbs. Any framework region can be used with the CDRs as described herein.

In some embodiments, one or more of the sdAbs for incorporation into the IL10R binding molecules is a chimeric sdAb, in which the CDRs are derived from one species (e.g., camel) and the framework and/or constant regions are derived from another species (e.g., human or mouse). In specific embodiments, the framework regions are human or humanized sequences. Thus, IL10R binding molecules comprising one or more humanized sdAbs are considered within the scope of the present disclosure.

In some embodiments, one or more of the sdAb of the cytokine receptor binding molecules of the present disclosure is a VHH. As used herein, the term “VHH” refers to a single domain antibody derived from camelid antibody typically obtained from immunization of camelids (including camels, llamas and alpacas (see, e.g., Hamers-Casterman, et al. (1993) Nature 363:446-448). VHHs are also referred to as heavy chain antibodies or Nanobodies® as Single domain antibodies may also be derived from non-mammalian sources such as VHHs obtained from IgNAR antibodies immunization of cartilaginous fishes including, but not limited to, sharks. A VHH is a type of single-domain antibody (sdAb) containing a single monomeric variable antibody domain. Like a full-length antibody, it is able to bind selectively to a specific antigen.

The complementary determining regions (CDRs) of VHHs are within a single-domain polypeptide. VHHs can be engineered from heavy-chain antibodies found in camelids. An exemplary VHH has a molecular weight of approximately 12-15 kDa which is much smaller than traditional mammalian antibodies (150-160 kDa) composed of two heavy chains and two light chains. VHHs can be found in or produced from Camelidae mammals (e.g., camels, llamas, dromedary, alpaca, and guanaco) which are naturally devoid of light chains. Descriptions of sdAbs and VHHS can be found in, e.g., De Greve et al., Curr Opin Biotechnol. 61:96-101, 2019; Ciccarese, et al., Front Genet. 10:997, 2019; Chanier and Chames, Antibodies (Basel) 8(1), 2019; and De Vlieger et al., Antibodies (Basel) 8(1), 2018. The CDRs derived from camelid VHHs may be used to prepare CDR-grafted VHHs which may be incorporated in the IL10R binding molecules.

In some embodiments, the VHH for incorporation into the IL10R binding molecule of the present disclosure is a humanized VHH containing human framework regions. The techniques for humanization of camelid single domain antibodies are well known in the art. See, e.g., Vincke, et al. (2009) General Strategy to Humanize a Camelid Single-domain Antibody and Identification of a Universal Humanized Nanobody Scaffold J. Biol. Chem. 284(5)3273-3284. Human framework regions useful in the preparation of humanized VHHs include, but are not limited to, VH3-23 (e.g., UniProt ID: P01764), VH3-74 (e.g., UniProt ID: A0A0B4J1X5), VH3-66 (e.g., UniProt ID: A0A0C4DH42), VH3-30 (e.g., UniProt ID: P01768), VH3-11 (e.g., UniProt ID: P01762), and VH3-9 (e.g., UniProt ID: P01782).

### Stably Associated:

The IL10R binding molecules of the present disclosure comprise a single domain antibody that selectively binds to the extracellular domain of IL10Ra (an “IL10Ra sdAb”) in stable association with a single domain antibody that selectively binds to the extracellular domain of IL10Rb (an “IL10Rb sdAb”). As used herein, the term “stably associated” or “in stable association with” are used to refer to the various means by which one molecule (e.g., a polypeptide) may be thermodynamically and/or kinetically associated with another molecule. The stable association of one molecule to another may be achieved by a variety of means, including covalent bonding and non-covalent interactions.

In some embodiments, stable association of the IL10Ra sdAb and IL10Rb sdAb may be achieved by a covalent bond such as peptide bond. In some embodiments, the covalent linkage between the first and second binding domains is a covalent bond between the C-terminus of the first binding domain and the N-terminus of the second binding domain.

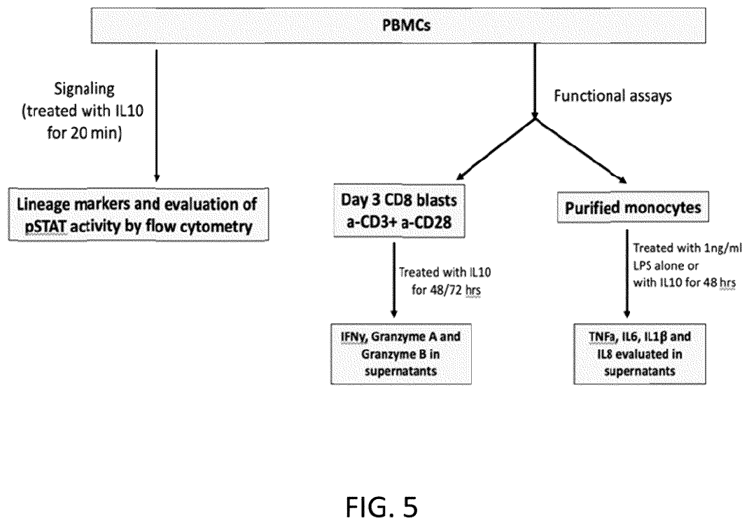

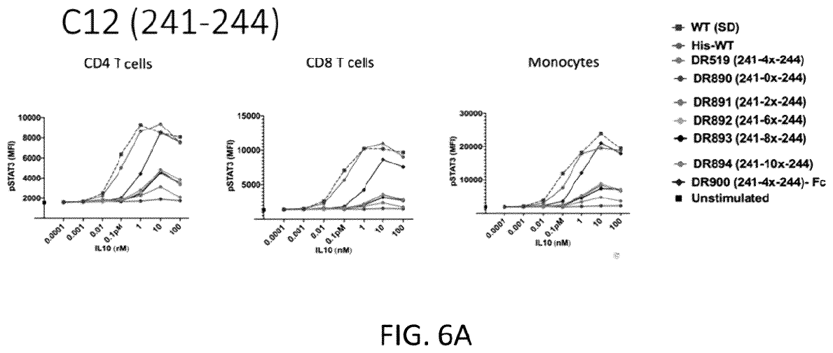

In some embodiments, the covalent linkage of the IL10Ra sdAb and IL10Rb sdAb of the IL10R binding protein is effected by a coordinate covalent linkage. The present disclosure provides examples of single domain antibodies comprising a chelating peptide. The chelating peptide results in a coordinate covalent linkage to a transition metal ion. In some embodiments, a transition metal ion is capable of forming a coordinate covalent linkage with two or more chelating peptides. Consequently, the first and second binding domains may each comprise a chelating peptide and a stable association of the binding domains by each subunit forming a coordinate covalent complex with a transition metal ion. In some embodiments, the transition metal ion is selected from vanadium, manganese, iron, iridium, osmium, rhenium platinum, palladium, cobalt, chromium or ruthenium. A schematic illustration of this configuration is provided in FIG. 4B, of the attached drawings. It should be noted that in each of the configurations illustrated in FIGS. 4A-4B, the N-terminal domain of the single domain antibody is presented to the environment enabling facilitating enhanced exposure of the CDRs of the sdAb to the target cytokine receptor ECD. The formation of the coordinate covalent linkage between the is favored when the transition metal ion is in a kinetically labile oxidation state, for example Co(II), Cr(II), or Ru(III). Following complexation, the oxidation state of the transition metal may be changed (oxidized or reduced) to a kinetically inert oxidation state, for example Co(III), Cr(III), or Ru(II), provide a kinetically inert coordinate covalent complex. The the formation of kinetically inert and kinetically labile coordinate covalent complexes between proteins comprising chelating peptides via a transition metal are described in more detail in Anderson, et al. U.S. Pat. No. 5,439,928 issued Aug. 8, 1995.

In some embodiments, the covalent linkage of the IL10Ra sdAb and IL10Rb sdAb of tie IL10R binding molecule may further comprise a linker. Linkers are molecules selected from selected from the group including, but not limited to, peptide linkers and chemical linkers. In some embodiments, the linker a joins the C-terminus of the IL10Ra sdAb to the N-terminus of the IL10Rb sdAb. In some embodiments, the linker joins the C-terminus of the IL10Rb sdAb to the N-terminus of the IL10Ra sdAb.

In some embodiments, the linker is a peptide linker. A peptide linker can include between 1 and 50 amino acids (e.g., between 2 and 50, between 5 and 50, between 10 and 50, between 15 and 50, between 20 and 50, between 25 and 50, between 30 and 50, between 35 and 50, between 40 and 50, between 45 and 50, between 2 and 45, between 2 and 40, between 2 and 35, between 2 and 30, between 2 and 25, between 2 and 20, between 2 and 15, between 2 and 10, between 2 and 5 amino acids). Glycine and glycine-serine polymers are relatively unstructured, and therefore may serve as a neutral tether between components. Examples of glycine polymers include (G)n, glycine-alanine polymers, alanine-serine polymers, glycine-serine polymers (for example, (GmSo)n (SEQ ID NO: 562), (GSGGS)n (SEQ ID NO: 563), (GmSoGm)n (SEQ ID NO: 564), (GmSoGmSoGm)n (SEQ ID NO: 565), (GSGGSm)n (SEQ ID NO: 566), (GSGSmG)n (SEQ ID NO: 567) and (GGGSm)n (SEQ ID NO: 568), and combinations thereof, where m, n, and o are each independently selected from an integer of at least 1 to 20, e.g., 1-18, 216, 3-14, 4-12, 5-10, 1, 2, 3, 4, 5, 6, 7, 8, 9, or 10), and other flexible linkers. Exemplary flexible peptide linkers useful in the preparation of the IL10R binding molecules of the present disclosure include, but are note limited to, to the linkers provided in Table 16.

In some embodiments, the covalent linkage of the first and second domains may be achieved by a chemical linker. Examples of chemical linkers include aryl acetylene, ethylene glycol oligomers containing 2-10 monomer units, diamines, diacids, amino acids, or combinations thereof.

In some embodiments, stable association the IL10Ra sdAb and IL10Rb sdAb of the IL10R binding protein is be effected by non-covalent interaction. Examples of non-covalent interactions that provide a stable association between two molecules include electrostatic interactions (e.g., hydrogen bonding, ionic bonding, halogen binding, dipole-dipole interactions, Van der Waals forces and p-effects including cation-p interactions, anion-p interactions and p-p interactions) and hydrophobic/hydrophilic interactions. In some embodiments, the stable association of sdAbs of the binding molecules of the present disclosure may be effected by non-covalent interactions.

In one embodiment, the non-covalent stable association of the IL10Ra sdAb and IL10Rb sdAb of the IL10R binding molecule may be achieved by conjugation a sdAb each monomer of a “knob-into-hole” engineered Fc dimer. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g., tyrosine or tryptophan) creating a projection from the surface (“knob”) and ii) in the CH3 domain of a second heavy chain, an amino acid residue is replaced with an amino acid residue having a smaller side chain (e.g., alanine or threonine), thereby generating a cavity (“hole”) within at interface in the second CH3 domain within which the protruding side chain of the first CH3 domain (“knob”) is received by the cavity in the second CH3 domain. The knob-into-hole modification is more fully described in Ridgway, et al. (1996) Protein Engineering 9(7):617-621 and U.S. Pat. No. 5,731,168, issued Mar. 24, 1998, U.S. Pat. No. 7,642,228, issued Jan. 5, 2010, U.S. Pat. No. 7,695,936, issued Apr. 13, 2010, and U.S. Pat. No. 8,216,805, issued Jul. 10, 2012. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 and Y349 which results in a stabilizing disulfide bridge between the two antibody heavy chains in the Fe region (Carter, et al. (2001) Immunol Methods 248, 7-15). The knob-into-hole format is used to facilitate the expression of a first polypeptide (e.g., an IL10Rb binding sdAb) on a first Fc monomer with a “knob” modification and a second polypeptide on the second Fc monomer possessing a “hole” modification to facilitate the expression of heterodimeric polypeptide conjugates. The knob-into-hole format is used to facilitate the expression of a first polypeptide on a first Fc monomer with a “knob” modification and a second polypeptide on the second Fc monomer possessing a “hole” modification to facilitate the expression of heterodimeric polypeptide conjugates. One embodiment of an IL10R binding molecule wherein the IL10Ra sdAb and IL10Rb sdAb are in stable, non-covalent association is wherein the each sdAb of the IL10R binding molecule covalently bonded, optionally including a linker, to each subunit of the knob-into-hole Fc dimer as illustrated in FIG. 4A, of the attached drawings.

Generation and Evaluation of IL10Ra sdAbs

To generate sdAbs against the hIL2Ra, the extracellular domain of the hIL2Ra protein may be used an immunogen. The extracellular domain of the mature (lacking the signal sequence) hIL2Ra possesses the amino acid sequence (amino acids 22-235 of SEQ ID NO:452) has the amino acid sequence

(SEQ ID NO: 453)
HGTELPSPPSVWFEAEFFHHILHWTPIPNQSESTCYEVALLRYGIESWN
SISNCSQTLSYDLTAVTLDLYHSNGYRARVRAVDGSRHSNWTVTNTRFS
VDEVTLTVGSVNLEIHNGFILGKIQLPRPKMAPANDTYESIFSHFREYE
IAIRKVPGNFTFTHKKVKHENFSLLTSGEVGEFCVQVKPSVASRSNKGM
WSKEECISLTRQYFTVTN

In some embodiments, when employed as an immunogen or a immunogenic composition, the hIL2Ra ECD may be provided as a domain of a fusion protein with an immunomodulatory protein.

To generate sdAbs against the mIL10Ra, the extracellular domain of the mIL10Ra protein may be used an immunogen. The extracellular domain of the extracellular domain of mIL10Ra possesses the amino acid sequence (amino acids 17-241 of SEQ ID NO:454) has the amino acid sequence

(SEQ ID NO: 455)
LEFIAYGTELPSPSYVWFEARFFQHILHWKPIPNQSESTYYEVALKQYG
NSTWNDIHICRKAQALSCDLTTFTLDLYHRSYGYRARVRAVDNSQYSNW
TTTETRFTVDEVILTVDSVTLKAMDGIIYGTIHPPRPTITPAGDEYEQV
FKDLRVYKISIRKFSELKNATKRVKQETFTLTVPIGVRKFCVKVLPRLE
SRINKAEWSEEQCLLITTEQYFTVTNLSI

In some embodiments, when employed as an immunogen or a immunogenic composition, the mIL2Ra ECD may be provided as a domain of a fusion protein with an immunomodulatory protein.

A series of hIL10Ra sdAbs were generated in substantial accordance with the teaching of Examples 1-4 herein. Briefly, a camel was sequentially immunized with the ECD of the human IL10Ra over a period several weeks of by the subcutaneous an adjuvanted composition containing a recombinantly produced fusion proteins comprising the extracellular domain of the IL10Ra, the human IgG1 hinge domain and the human IgG1 heavy chain Fc. Following immunization, RNAs extracted from a blood sample of appropriate size VHH-hinge-CH2-CH3 species were transcribed to generate DNA sequences, digested to identify the approximately 400 bp fragment comprising the nucleic acid sequence encoding the VHH domain was isolated. The isolated sequence was digested with restriction endonucleases to facilitate insertion into a phagemid vector for in frame with a sequence encoding a his-tag and transformed into E. coli to generate a phage library. Multiple rounds of bio-panning of the phage library were conducted to identify VHHs that bound to the ECD of IL10Ra (human or mouse as appropriate). Individual phage clones were isolated for periplasmic extract ELISA (PE-ELISA) in a 96-well plate format and selective binding confirmed by colorimetric determination. The IL10Ra binding molecules that demonstrated specific binding to the IL10Ra antigen were isolated and sequenced and sequences analyzed to identify VHH sequences, CDRs and identify unique VHH clonotypes. As used herein, the term “clonotypes” refers a collection of binding molecules that originate from the same B-cell progenitor cell, in particular collection of antigen binding molecules that belong to the same germline family, have the same CDR3 lengths, and have 70% or greater homology in CDR3 sequence.

The amino acid sequences of VHH molecules demonstrating specific binding to the hIL10Ra ECD antigen (hIL10Ra VHHs) are provided in Table 5 and the CDRs isolated from such VHHs are provided in Table 2. Nucleic acid sequences encoding the VHHs of Table 5 are provided in Table 8.

To confirm and evaluate binding affinities of the binding of the IL10Ra sdAbs, a representative example from each clonotype generated was selected for evaluation of binding via SPR. Evaluation of binding affinity of the hIL10Ra VHHs for ECD corresponding to SEQ ID NOS 159, 161, 162, 163, 165, 167 and 170 was conducted using surface plasmon resonance (SPR) in substantial accordance with the teaching of Example 5. Buffer-subtracted sensograms were processed with Biacore T200 Evaluation Software and globally fit with a 1:1 Langmuir binding model (bulk shift set to zero) to extract kinetics and affinity constants (kₐ, k_{d}, K_{D}). R_{MAX}<100 RU indicates surface density compatible with kinetics analysis. Calculated Rₘₐₓ values were generated using the equation: Rₘₐₓ=Load (RU)×valency of ligand×(Molecular weight of analyte/Molecular weight of ligand). Surface activity was defined as the ratio of experimental/calculated Rmax. The results of these binding affinity experiments are provided in Table 22 below. The data provided in Table 22 demonstrats that the IL10Ra single domain antibodies generated possessed specific binding to the ECD of hIL10Ra.

TABLE 22
anti-hIL10Ra Mono-Fc VHHs binding to hIL10Ra-
his (Antigen: Sino Biological, Catalog#10419)

| | | | | | | | Calc. | |
|---|---|---|---|---|---|---|---|---|
| | SEQ | k_{ON} | k_{OFF} | Affinity | Rmax | Load | Rmax | Surface |
| Ligand | ID NO | (1/Ms) | (1/s) | (nM) | (RU) | (RU) | (RU) | Activity |
| hIL-10Ra_VHH6 | 159 | 1.06E+05 | 1.90E−04 | 1.8 | 31.8 | 50.2 | 38 | 83% |
| hIL-10Ra_VHH8 | 161 | 1.68E+05 | 1.07E−03 | 6.4 | 17.7 | 40.6 | 31 | 57% |
| hIL-10Ra_VHH9 | 162 | 9.97E+04 | 1.15E−03 | 11.6 | 42.2 | 83.2 | 64 | 66% |
| hIL-10Ra_VHH10 | 163 | 1.04E+05 | 1.39E−03 | 13.4 | 106 | 242 | 185 | 57% |
| hIL-10Ra_VHH12 | 165 | 1.54E+05 | 1.19E−03 | 7.7 | 18.3 | 32.9 | 25 | 73% |
| hIL-10Ra_VHH14 | 167 | 9.84E+04 | 1.36E−04 | 1.4 | 17.1 | 44.1 | 34 | 51% |
| hIL-10Ra_VHH17 | 170 | 3.27E+05 | 1.97E−03 | 6 | 48.4 | 180 | 137 | 35% |

Table 2 provides CDRs useful in the preparation of IL10Ra sdAbs for incorporation into the binding molecules of the present disclosure. In some embodiments, the IL10Ra sdAbs are generated in response to immunization with the and specifically bind to the ECD of hIL10Ra. In some embodiments, the IL10Ra sdAb is a single domain antibody comprising: a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 1, 4, 7, 10, 13, 16, 19, 22, 25, 28, 31, 34, 37, 40, 43, 46, 49 and 52; a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 2; 5, 8, 11, 14, 17, 20, 23, 26 29, 32, 35, 38, 41, 44, 47, 50 and 53; and a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 3, 6, 9, 12, 15, 21, 24, 27, 30, 33, 36, 39, 42, 45, 48, 51 and 54.

In some embodiments, the IL10Ra sdAb comprises a sequence having at least 90% (e.g., 91%, 92%, 93%, 94%, 95%, 96%, 97%, 98%, 99%, or 100%) sequence identity to a sequence of any one the of amino acid sequences of hIL10Ra sdAbs provided in Table 5 (SEQ ID NOS: 154-171) In certain embodiments, the IL10Ra sdAb comprises sequence that is substantially identical to a sequence of any one the of amino acid sequences of hIL10Ra sdAbs provided in Table 5 (SEQ ID NOS: 154-171). In certain embodiments, the IL10Ra sdAb comprises sequence that is identical to a sequence of any one the of amino acid sequences of hIL10Ra sdAbs provided in Table 5 (SEQ ID NOS: 154-171).

In another aspect, the disclosure provides an isolated nucleic acid encoding IL10Ra sdAb described herein. Table 8 provides DNA sequences (SEQ ID NOS: 205-222 encoding the IL10Ra sdAbs of Table 5 (SEQ ID NOS: 154-171). In certain embodiments, the present disclosure provides an isolated nucleic acid comprising a sequence having at least 90% (e.g., 91%, 92%, 93%, 94%, 95%, 96%, 97%, 98%, 99%, or 100%) sequence identity to a DNA sequence of Table 8 (SEQ ID NOS: 205-222). In certain embodiments, the present disclosure provides an isolated nucleic acid comprising a DNA sequence that is substantially identical to a DNA sequence of Table 8 (SEQ ID NOS: 205-222). In certain embodiments, the present disclosure provides an isolated nucleic acid comprising a DNA sequence that is identical to a DNA sequence of Table 8 (SEQ ID NOS: 205-222).

### Generation and Evaluation of IL10Rb VHHs

The present disclosure further provides IL10Rb sdAbs that specifically bind to the extracellular domain of the mouse and human IL10Rb (hIL10Rb) and mouse IL10Rb (mIL10Rb). The procedure employed for the generation of IL10Rb VHHs was substantially the same as provided above and in substantial accordance with the teaching of Example 5 with the exception of the antigen used for immunization as discussed in more detail below.

To generate sdAbs against the hIL10Rb, the extracellular domain of the hIL10Rb protein was used as the immunogen. The extracellular domain of mature (lacking the signal sequence) hIL10Rb possesses the amino acid sequence:

(SEQ ID NO: 457)
MVPPPENVRMNSVNFKNILQWESPAFAKGNLTFTAQYLSYRIFQDKCMN
TTLTECDFSSLSKYGDHTLRVRAEFADEHSDWVNITFCPVDDTIIGPPG
MQVEVLADSLHMRFLAPKIENEYETWTMKNVYNSWTYNVQYWKNGTDEK
FQITPQYDFEVLRNLEPWTTYCVQVRGFLPDRNKAGEWSEPVCEQTTHD
ETVPS

To generate sdAbs against mIL10Rb, the extracellular domain of the mIL10Rb protein was used as an immunogen. The extracellular domain of the mature (lacking the signal sequence) mIL10Rb possesses the amino acid sequence the extracellular domain of the hIL10Rb protein was used as the immunogen. The extracellular domain of mature (lacking the signal sequence) mIL10Rb possesses the amino acid sequence:

(SEQ ID NO: 459)
MIPPPEKVRMNSVNFKNILQWEVPAFPKTNLTFTAQYESYRSFQDHCK
RTASTQCDFSHLSKYGDYTVRVRAELADEHSEWVNVTFCPVEDTIIGP
PEMQIESLAESLHLRFSAPQIENEPETWTLKNIYDSWAYRVQYWKNGT
NEKFQVVSPYDSEVLRNLEPWTTYCIQVQGFLLDQNRTGEWSEPICER
TGNDEITPS

The amino acid sequences of VHH molecules generated in accordance with the foregoing and demonstrating specific binding to the hIL10Rb ECD antigen (hIL10Rb VHHs) are provided in Table 6 (SEQ ID NOS: 172-198) and the CDRs isolated from such VHHs are provided in Table 3 (SEQ ID NOS: 55-135). Nucleic acid sequences encoding the VHHs of Table 6 are provided in Table 9 (SEQ ID NOS: 223-249).

The amino acid sequences of VHH molecules generated in accordance with the foregoing and demonstrating specific binding to the mIL10Rb ECD antigen (mIL10Rb VHHs) are provided in Table 7 (SEQ ID NOS: 199-204) and the CDRs isolated from such VHHs are provided in Table 4 (SEQ ID NOS: 136-153). Nucleic acid sequences encoding the VHHs of Table 7 are provided in Table 10 (SEQ ID NOS: 250-255).

To confirm and evaluate binding affinities of the binding of the hIL10Rb sdAbs, a representative example from each clonotype generated was selected for evaluation of binding via SPR. Evaluation of binding affinity of the hIL10Rb binding molecules for hIL10Rb corresponding to SEQ ID NOS: 172, 174, 183, 186 187, 197 and 198 was conducted using surface plasmon resonance (SPR) in substantial accordance with the teaching of Example 5 Buffer-subtracted sensograms were processed with Biacore T200 Evaluation Software and globally fit with a 1:1 Langmuir binding model (bulk shift set to zero) to extract kinetics and affinity constants (ka, kd, KD). RMAX<100 RU indicates surface density compatible with kinetics analysis. Calculated Rmax values were generated using the equation: Rmax=Load (RU)×valency of ligand×(Molecular weight of analyte/Molecular weight of ligand). Surface activity was defined as the ratio of experimental/calculated Rmax. The results of these binding affinity experiments are provided in Table 23 below.

TABLE 23
hIL10Rb Mono-Fc VHHs binding to hIL10Rb-his
(Antigen: Sino Biological, Catalog#10945)

| | | | | | | | Calc. | |
|---|---|---|---|---|---|---|---|---|
| | SEQ | k_{ON} | k_{OFF} | Affinity | Rmax | Load | Rmax | Surface |
| Ligand | ID | (1/Ms) | (1/s) | (nM) | (RU) | (RU) | (RU) | Activity |
| hIL-10Rb_VHH1 | 172 | 1.75E+05 | 1.44E−03 | 8.3 | 45.6 | 72.9 | 56 | 82% |
| hIL-10Rb_VHH12 | 183 | 1.11E+05 | 1.43E−03 | 12.9 | 28.1 | 54.6 | 42 | 67% |
| hIL-10Rb_VHH3 | 174 | 9.29E+04 | 2.14E−02 | 231 | 22.4 | 56.7 | 43 | 52% |
| hIL-10Rb_VHH15 | 186 | 7.18E+04 | 8.66E−03 | 121 | 28.9 | 210 | 160 | 18% |
| hIL-10Rb_VHH16 | 187 | 2.93E+03 | 2.21E−02 | 7540 | 227* | 54.4 | 42 | 545%* |
| hIL-10Rb_VHH26 | 197 | 6.78E+04 | 2.13E−02 | 314 | 25.6 | 62.2 | 47 | 54% |
| hIL-10Rb_VHH27 | 198 | 1.07E+07 | 2.54E−02 | 2.4 | 36.3 | 198 | 151 | 24% |

*Inaccurate fit

The data provided demonstrates that the hIL10Rb single domain antibodies generated possessed specific binding to the ECD of hIL10Rb and a range of binding affinities useful in the modulation of the activity of IL10R binding molecules as discussed below.

Tables 3 and 4 provides CDRs useful in the preparation of IL10Ra sdAbs for incorporation into the binding molecules of the present disclosure. In some embodiments, the IL10Ra sdAbs are generated in response to immunization with the and specifically bind to the ECD of hIL10Ra. In some embodiments, the IL10Ra sdAb is a single domain antibody comprising: a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to a CDR1 sequence of Table 3; a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to a CDR1 sequence of Table 3; and a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to a CDR1 sequence of Table 3. In some embodiments, the IL10Ra sdAbs are generated in response to immunization with the and specifically bind to the ECD of mIL10Ra. In some embodiments, the IL10Ra sdAb is a single domain antibody comprising: a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to a CDR1 sequence of Table 4; a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to a CDR1 sequence of Table 4; and a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to a CDR1 sequence of Table 4.

In some embodiments, the IL10Rb sdAb comprises a sequence having at least 90% (e.g., 91%, 92%, 93%, 94%, 95%, 96%, 97%, 98%, 99%, or 100%) sequence identity to a sequence of any one the of amino acid sequences of an hIL10Rb sdAb of Table 6 (SEQ ID NOS: 172-198). In certain embodiments, the IL10Rb sdAb comprises sequence that is substantially identical to a sequence of any one the of amino acid sequences of an hIL10ba sdAb of Table 6 (SEQ ID NOS: 172-198). In certain embodiments, the IL10Rb sdAb comprises sequence that is identical to a sequence of any one the of amino acid sequences of an hIL10ba sdAb of Table 6 (SEQ ID NOS: 172-198).

In some embodiments, the IL10Rb sdAb comprises a sequence having at least 90% (e.g., 91%, 92%, 93%, 94%, 95%, 96%, 97%, 98%, 99%, or 100%) sequence identity to a sequence of any one the of amino acid sequences of an mIL10Rb sdAb of Table 7 (SEQ ID NOS: 199-204). In certain embodiments, the IL10Rb sdAb comprises sequence that is substantially identical to a sequence of any one the of amino acid sequences of an mIL10Rb sdAb of Table 7 (SEQ ID NOS: 199-204). In certain embodiments, the IL10Rb sdAb comprises sequence that is identical to a sequence of any one the of amino acid sequences of mIL10Rb sdAb of Table 7 (SEQ ID NOS: 199-204).

In another aspect, the disclosure provides an isolated nucleic acid encoding IL10Rb sdAb described herein. In certain embodiments, the present disclosure provides an isolated nucleic acid comprising a sequence having at least 90% (e.g., 91%, 92%, 93%, 94%, 95%, 96%, 97%, 98%, 99%, or 100%) sequence identity to a DNA sequence of Table 9 (SEQ ID NOS: 223-249). In certain embodiments, the present disclosure provides an isolated nucleic acid comprising a DNA sequence that is substantially identical to a DNA sequence of Table 9 (SEQ ID NOS: 223-249). In certain embodiments, the present disclosure provides an isolated nucleic acid comprising a DNA sequence that is identical to a DNA sequence of Table 9 (SEQ ID NOS: 223-249).

In another aspect, the disclosure provides an isolated nucleic acid encoding IL10Rb sdAb described herein. In certain embodiments, the present disclosure provides an isolated nucleic acid comprising a sequence having at least 90% (e.g., 91%, 92%, 93%, 94%, 95%, 96%, 97%, 98%, 99%, or 100%) sequence identity to a DNA sequence of Table 10 (SEQ ID NOS: 250-255). In certain embodiments, the present disclosure provides an isolated nucleic acid comprising a DNA sequence that is substantially identical to a DNA sequence of Table 10 (SEQ ID NOS: 250-255). In certain embodiments, the present disclosure provides an isolated nucleic acid comprising a DNA sequence that is identical to a DNA sequence of Table 10 (SEQ ID NOS: 250-255).

### Generation of IL10R Polypeptide Binding Molecules

In some embodiments, the IL10R binding molecule of the present invention is a polypeptide of the following formula [#1]:

H2N-(IL10 VHH #1)-(L1)ₐ-(IL10 VHH #2)-(L2)_{b}-(CP)_{c}-COOH [#1]

wherein: “-” represents a covalent bond; L1 and L2 are linkers; CP is a chelating peptide; a, b, and c are independently selected from the integers 0 or 1; “H2N” denotes the amino terminus; and “COOH” denotes the carboxy terminus of the polypeptide.

Using the hIL10Ra VHHs and hIL10Rb VHHs prepared above, a series of ninety-eight polypeptide IL10R binding molecules of formula [#1] were prepared as SEQ ID NOS: 256-353 and with respect to each of SEQ ID NOS 256-353: a=1 and L1=G3S (SEQ ID NO: 484); b=1 and L2=Ala-Ser; c=1 and CP=hexa-histidine (SEQ ID NO: 532). The IL10Ra VHH, linkers and IL10Ra VHH, chelating peptide elements of these ninety-eight IL-10 receptor binding proteins of SEQ ID NOS 256-353 is provided in Table 11. The amino acid sequences of SEQ ID NOS: 256-353 are provided in Table 12 and the DNA sequence encoding each of the foregoing used for expression of the IL10R binding molecules is provided in Table 14 (SEQ ID NOS: 353-402).

The IL10R binding proteins of Tables 11 and 12 were prepared and evaluated for IL10 activity. Details regarding the expression and purification of these 98 IL10R binding proteins is provided in the Examples. Briefly, nucleic acid sequences encoding SEQ ID Nos: 256-353 were synthesized as SEQ ID Nos: 354-451, respectively, and were inserted into a recombinant expression vector and expressed in HEK293 cells in 24 well plate format and purified in substantial accordance with Example 4. The supernatants containing the IL-10 receptor binding proteins of SEQ ID NOS: 192-298 were evaluated for activity with unstimulated and wild-type human IL-10 as controls in substantial accordance with Examples 5 and 6 herein. The results of these experiments are provided in Table 13.

As can be seen from the data provided in Table 13, IL-10 receptor binding proteins of the formula [#1] demonstrated IL-10 activity in the IL-10 activity assay. The level of IL-10 activity was categorized as low (above unstimulated and A₆₃₀<1), medium (A₆₃₀ 1-1.5) and high (A₆₃₀>1.5) based on absorbance readings. From the above data, 11 IL10 receptor binding proteins demonstrated high activity (SEQ ID NOs: 258, 273, 274, 275, 276, 282, 290, 297, 298, 308 and 314), 4 with medium activity (SEQ ID NOs: 267, 269, 271, and 333) and 8 VHHs with low activity (SEQ ID NOs: 276, 281, 283, 288, 291, 301, 303, and 313).

To further investigate and characterize the binding of the IL10R binding molecules prepared above, a selection of the foregoing molecules was evaluated for binding to the hIL10Ra and hIL10Rb receptor subunits by SPR in substantial accordance with the teaching of the Examples and the data presented in Table 24 and 25 below. The first column represents the VHHs used in the IL10R binding molecule (as provided in Table 5 and 6 above) and their orientation from amino to carboxy, and each molecule was expressed with a four amino acid G3S linker (SEQ ID NO: 484) between the VHHs conjugated to biotin. As illustrated by the data provided in Tables 24 and 25 below, the IL10R polypeptide binding molecules of the formula [#1] demonstrate single-digit nanomolar binding affinity to each of the hIL10Ra and hIL10Rb VHH subunits as determined by SPR. This data demonstrates that each of the hIL10Ra and hIL10Rb VHH subunits retains their respective binding affinity to their respective IL10 receptor subunits and that, as discussed in more detail below, retained activity whether provided in the “forward” or “reverse” configurations.

TABLE 24
Anti-hIL10Ra/hIL10Rb dual VHH binding to hIL10Ra-his
(Analyte = hIL10Ra his, Sino Biological cat#10419-H08H)

| | | | | | | Calc. | |
|---|---|---|---|---|---|---|---|
| Ligand | k_{ON} | k_{OFF} | Affinity | Rmax | Load | Rmax | |
| (IL10R VHHs) | (1/Ms) | (1/s) | (nM) | (RU) | (RU) | (RU) | Activity |
| hIL10Rb_VHH1- | 2.5E+05 | 2.7E−04 | 1.1 | 67.8 | 93 | 130.2 | 52% |
| hIL10Ra_VHH9- | | | | | | | |
| biotin | | | | | | | |
| hIL10Rb_VHH12- | 1.8E+05 | 6.5E−05 | 0.35 | 41.6 | 53 | 74.2 | 56% |
| hIL10Ra_VHH8 | | | | | | | |
| biotin | | | | | | | |
| IL10 VHH DR509 | 4.9E+05 | 1.3E−04 | 0.2 | 51.5 | 85 | 119 | 43% |
| biotin | | | | | | | |
| hIL10Ra_VHH17- | N/A | N/A | <5 | N/A | 53 | N/A | N/A |
| hIL10Rb_VHH16 | | | | | | | |
| biotin | | | | | | | |

TABLE 25
hIL10Ra/hIL10Rb dual VHH binding to hIL10Rb-his
(Analyte = Sino Biological cat#10945-H08H)

| | | | | | | Calc. | |
|---|---|---|---|---|---|---|---|
| Ligand | k_{ON} | k_{OFF} | Affinity | Rmax | Load | Rmax | |
| (IL10R VHHs) | (1/Ms) | (1/s) | (nM) | (RU) | (RU) | (RU) | Activity |
| hIL10Ra_VHH17- | 1.6E+05 | 4.5E−04 | 2.9 | 30 | 62 | 86.8 | 34% |
| hIL10Rb_VHH16 | | | | | | | |
| biotin | | | | | | | |
| hIL10Rb_VHH12- | 4.4E+05 | 6.8E−04 | 1.5 | 32 | 53 | 74.2 | 42% |
| hIL10Ra_VHH8 biotin | | | | | | | |
| hIL10Rb_VHH1- | 6.6E+05 | 1.8E−03 | 2.7 | 31 | 90 | 126 | 25% |
| hIL10Ra_VHH9 biotin | | | | | | | |
| hIL10Ra_VHH12- | 1.6E+06 | 3.0E−03 | 1.9 | 100 | 84 | 117.6 | 85% |
| hIL10Rb_VHH27 | | | | | | | |
| biotin | | | | | | | |

### Modulation of Activity of Receptor Binding Molecules

In some embodiments, such as to achieve partial agonism or selective activation of particular cell types, the design of the IL10R binding molecules of the present disclosure may be modulated by structural variations in the design of the receptor binding molecule. This variation in activity may be employed to modulate the binding and activity of the IL10R receptor binding molecule, for to optimize the activity of the IL10R binding molecule to achieve partial agonism, selective cell type activation or to provide molecules having increased or decreased binding relative to the cognate ligand for each of the IL10Ra sdAb and IL10Rb sdAb for their respective receptor subunits.

The ability to modulate activity of the IL10R binding molecules of the present disclosure provides substantial benefits in multiple therapeutic applications. IL10 is a pleiotropic cytokine that regulates multiple immune responses through actions on T cells, B cells, macrophages, and antigen presenting cells (APC) and seemingly paradoxical activity which has limited its clinical development. IL10 is reported to suppress immune responses and inhibits the expression of IL-1a, IL-1B, IL-6, IL-8, TNF-α, GM-CSF and G-CSF in activated monocytes and macrophages. IL10 is associated with suppression of IFN-γ production by NK cells. In contrast, IL10 exhibits immuno-stimulatory properties, including enhancing the stimulation IL-2- and IL-4-treated thymocytes, enhancing the viability of B cells, and stimulating the presentation of MHC class II antigens. As a result, the use of IL-10 has been identified as useful in the treatment of a broad range of diseases, disorders and conditions, including inflammatory conditions, immune-related disorders, fibrotic disorders, metabolic disorders and cancer.

The IL10R molecules of the present disclosure enable modulation of activity provide significant benefits in the treatment of human disease. IL10R binding proteins described herein are useful in the treatment of neoplastic diseases, such as cancer (e.g., a solid tumor cancer; e.g., non-small-cell lung carcinoma (NSCLC), renal cell carcinoma (RCC), or melanoma) in a subject in need thereof. In some embodiments, the IL10R binding protein described herein can provide a longer therapeutic efficacy (e.g., lower effective dose, reduced toxicity) than IL10. The IL10R binding molecules of the present disclosure can trigger different levels of downstream signaling in different cell types. For example, by varying the length of the linker between the IL10Ra sdAb antibody and the IL10Rb sdAb antibody in the IL10R binding molcule, the IL10R binding molecules provides a higher level of downstream signaling in desired cell types compared to undesired cell types. In some embodiments the IL10R binding molecules is a partial IL10 agonist that selectively activates T cells (e.g., CD8⁺ T cells) over macrophages. In some embodiments, activated T cells have an up-regulation of IFNgamma. In some embodiments, an IL10R binding protein that is a partial agonist can suppress autoimmune inflammatory diseases such as ulcerative colitis and Crohn's disease.

In one embodiment, the present disclosure provides an IL10Ra binding molecule that preferentially activates T cells, in particular CD8+ T cells, relative to monocytes. In one embodiment, the present disclosure provides an IL10Ra binding molecule of the formula #1 wherein the affinity of the IL10Ra sdAb has a higher affinity for the extracellular domain of IL10Ra than the affinity of the IL10Rb sdAb for the extracellular domain of IL10Rb. In some embodiments, the present disclosure provides a IL10Ra molecule of formula #1, wherein the affinity of the IL10Ra sdAb has an affinity for the extracellular domain of IL10Ra of from about 10⁻⁸ to about 10⁻¹⁰ M, alternatively from about 10⁻⁹ to about 10⁻¹⁰M, or alternatively about 10⁻¹⁰ M and the IL10Rb sdAb an affinity for the extracellular domain of IL10Rb of from about 10⁻⁶ to about 10⁻⁹ M, alternatively from about 10⁻⁷ to about 10⁻⁹M, alternatively from about 10⁻⁷ to about 10⁻⁸M, alternatively about 10⁻⁹M, alternatively about 10⁻⁸M. In some embodiments, the present disclosure provides a IL10Ra molecule of formula #1, wherein the affinity of the IL10Ra sdAb has an affinity for the extracellular domain of IL10Ra of from about 10⁻⁸ to about 10⁻¹⁰ M, alternatively from about 10⁻⁹ to about 10⁻¹⁰M, or alternatively about 10⁻¹⁰ M and the IL10Rb sdAb an affinity for the extracellular domain of IL10Rb of from about 10⁻⁶ to about 10⁻⁹ M, alternatively from about 10⁻⁷ to about 10⁻⁹M, alternatively from about 10⁻⁷ to about 10⁻⁸M, alternatively about 10⁻⁹M, alternatively about 10⁻⁸M, and the affinity of the IL10Ra sdAb for ECD of IL10Ra is more than 2 fold higher, alternatively more than 5 fold higher, alternatively more than 10 fold higher, alternatively more than 20 fold higher, alternatively more than 40 fold higher, alternatively more than 50 fold higher, alternatively more than 60 fold higher, alternatively more than 70 fold higher, alternatively more than 80 fold higher, alternatively more than 90 fold higher, alternatively more than 100 fold higher, alternatively more than 150 fold higher, alternatively more than 200 fold higher or alternatively more than 500 fold higher than the affinity of the IL10Rb sdAb for ECD of IL10Rb

In some embodiments, for example by varying the linker length, an IL10R binding molecule can cause a higher level of downstream signaling in T cells (e.g., CD8⁺ T cells) compared to the level of downstream signaling in macrophages, a cell type that expresses both IL10Ra and IL10Rb receptors but when activated too potently can cause anemia. When the downstream signaling in macrophages is activated to a high level, these activated macrophages can then eliminate aging red blood cells, causing anemia. The ability to modulate the activity of the IL10R binding molecule provides a molecule with a higher level of downstream signaling in T cells (e.g., CD8⁺ T cells) compared to the level of downstream signaling in macrophages, such that anemia is avoided. In some embodiments, the IL10R binding molecules of the present disclosure result in level of downstream signaling in T cells (e.g., CD8⁺ T cells) that is at least 1.1, 1.5, 2, 3, 5, or 10 times of the level of downstream signaling in macrophages. In other embodiments, different IL10Ra sdAb antibodies with different binding affinities and different IL10Rb sdAb antibodies with different binding affinities can be used to tune the activity of IL10R binding molecule. Further, when the IL10R binding molecule is provided as a single a polypeptide, the orientation of the two antibodies in the polypeptide can also be changed to make change the properties of the molecule. In some embodiments, it is desired to provide an the IL10R binding protein has a reduced Emax compared to the Emax caused by IL10, the cognate ligand for the IL10 receptor (i.e. an IL10R binding molecule that is a IL10 partial agonist). Emax reflects the maximum response level in a cell type that can be obtained by a ligand (e.g., a binding protein described herein or the cognate ligand (e.g., IL10)). In some embodiments, the IL10R binding protein described herein has at least 1% (e.g., between 1% and 100%, between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Emax caused by hIL10.

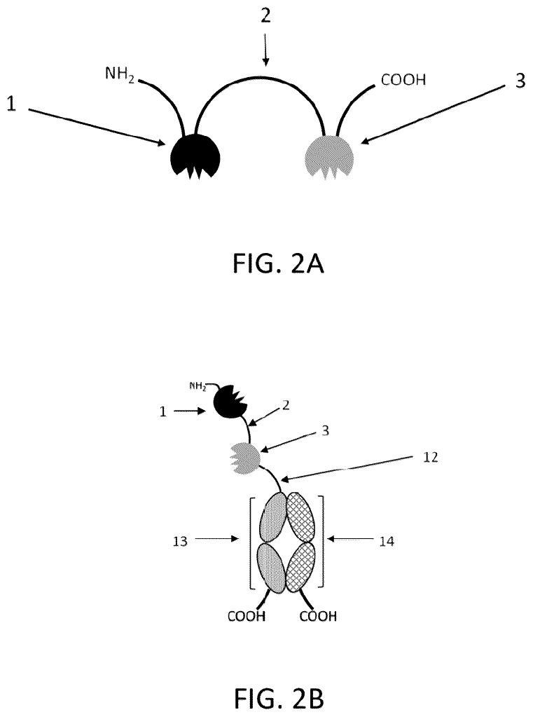

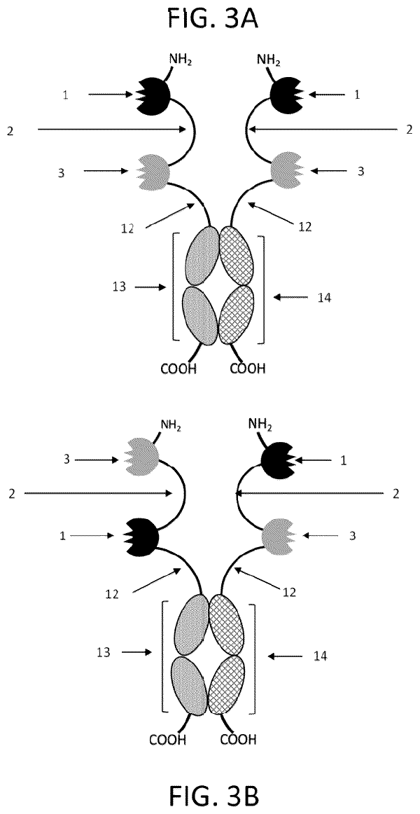

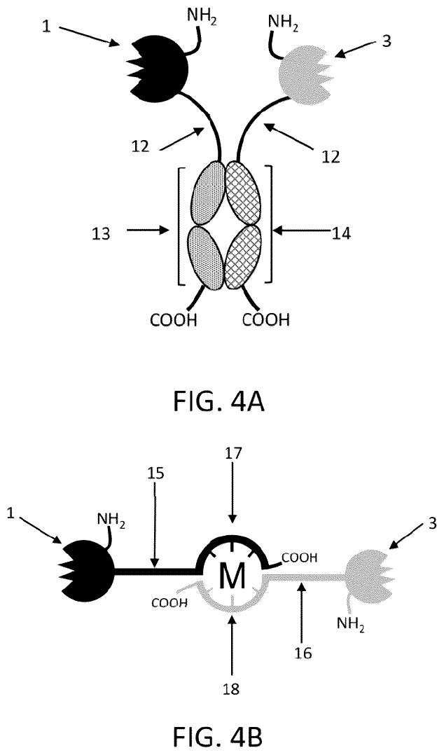

As previously discussed, the orientation of the sdAbs of the IL10R binding molecule may be used to optimize desired characteristics of the molecules. The orientation of the sdAbs of the IL10R my probided in a variety of different structures as illustrated in FIGS. 2, 3 and 4 of the attached drawings as expressed formulaically below:

H₂N-[IL10Ra sdAb]-[L1]ₓ-[IL10Rb sdAb]-[L2]_{y}-[CP]_{z}-COOH; (a)

H₂N-[IL10Rb sdAb]-[L1]ₓ-[IL10Ra sdAb]-[L2]_{y}-[CP]_{z}-COOH; (b)

H₂N-[IL10Ra sdAb]-[L1]ₓ-[IL10Rb sdAb]-[L2]_{y}-[Fc]_{z}-COOH; (c)

H₂N-[IL10Rb sdAb]-[L1]ₓ-[IL10Ra sdAb]-[L2]_{y}-[Fc]_{z}-COOH; (d)

wherein and L1 and L2 are indepently selected polypeptide linkers of 1-50 amino acids and x=0 or 1, y=0 or 1; and CP is a chelating peptide; “Fc” is a monomeric Fc domain and y=0 or 1; non-covalent complexes of the structure:

[H₂N-[IL10Ra sdAb]-[L1]ₓ-Fc1-COOH:H₂N-[IL10Rb sdAb]-[L2]_{y}-Fc2-COOH]; (e)

wherein and L1 and L2 are indepently selected polypeptide linkers of 1-50 amino acids and x=0 or 1, y=0 or 1; and CP is a chelating peptide; “Fc1” is a monomeric Fc domain “Fc2” is a monomeric Fc domain wherein Fc1 and Fc2 form a stable non-covalent association, and y=0 or 1; and coordinate covalent complexes of the structure

H₂N-[IL10Ra sdAb]-[L1]ₓ-(CP1)-M-(CP2)-(L2)-[IL10Rb sdAb]—NH₂ (f)

wherein and L1 and L2 are indepently selected polypeptide linkers of 1-50 amino acids and x and y are indepently selected from 0 or 1; and CP1 is a first chelaing peptide; CP2 is a second chelating peptide; and M is a transition metal ion.

Examples of the means by which the modulation of the activity and/or specificity of the receptor binding molecule of the present disclosure include, but are not limited to, altering the sequential orientation of the IL10Ra sdAb and IL10Rb sdAb in polypeptide IL10R binding molecules, independently varying the of the binding affinity of each IL10Ra sdAb and IL10Rb sdAbs with respect to their respective IL10Ra and/or each target, and modulating the distance between the IL10Ra sdAb and IL10Rb sdAbs, such as by employing linkers or varying lengths, which will affect the proximity of the intracellular signaling domains of the IL10 receptor subunits and thereby achieve modulation, of the intracellular signaling characteristic of the binding of the cognate ligand to the receptor, for example the level of phosphor-STAT3 induced in the cell. As illustrated by the data provided below, each of these variations may be used to tune the properties of IL10R binding molecules.

Sequential Orientations of IL10Ra and IL10Rb sdAbs in Single Polypeptide IL10R Binding Molecules

When the IL10R binding molecule comprises and IL10Ra sdAb and IL10Rb sdAb sequences in a single polypeptide, the IL10Ra sdAb and IL10Rb sdAb sequences may be arranged wherein the IL10Ra sdAb is N-terminal relative to the IL10Rb sdAb (termed herein as the “forward configuration”) or IL10Rb sdAb is N-terminal relative to the IL10Ra sdAb (termed herein as the “reverse configuration”). The two orientations can be illustrated as examples of the compound of formula [#1] represented by the following formulas;

H₂N-[IL10Ra sdAb]-[L1]ₓ-[IL10Rb sdAb]-[L2]_{y}-[CP]_{z}-COOH; (a)

H₂N-[IL10Rb sdAb]-[L1]ₓ-[IL10Ra sdAb]-[L2]_{y}-[CP]_{z}-COOH; (b)

wherein: “-” represents a covalent bond; L1 and L2 are linkers; CP is a chelating peptide; a, b, and c are independently selected from the integers 0 or 1; “H2N” denotes the amino terminus; and “COOH” denotes the carboxy terminus of the polypeptide.

### “Forward” Configuration:

In some embodiments, the IL10R binding molecule comprises a polypeptide of the structure of the formula [#1] wherein the N-terminal VHH of formula [#1] (i.e., IL10 VHH #1) is an IL10Ra VHH and the C-terminal VHH (i.e., IL10 VHH #2) is an IL10Rb VHH, and wherein: “-” represents a covalent bond; L1 and L2 are independtly selected polypeptide linkers of Table 16, linkers; CP is a chelating peptide; a, b, and c are independently selected from the integers 0 or 1; “H2N” denotes the amino terminus; and “COOH” denotes the carboxy terminus of the polypeptide.

### “Reverse” Configuration

In some embodiments, the IL10R binding molecule comprises a polypeptide of the structure of the formula [#1] wherein the N-terminal VHH of the above formula [#1] (i.e., IL10 VHH #1) is an anti-IL10Rb VHH and the C-terminal VHH (i.e., IL10 VHH #2) is an anti-IL10Ra VHH (“forward orientation”) and wherein: “-” represents a covalent bond; L1 and L2 are independtly selected polypeptide linkers of Table 16, linkers; CP is a chelating peptide; a, b, and c are independently selected from the integers 0 or 1; “H2N” denotes the amino terminus; and “COOH” denotes the carboxy terminus of the polypeptide.

### Evaluation of Activity of Exemplary IL10R Molecules of Forward and Revserse Orientations

IL10R binding molecules of forward and reverse orientation of the IL10Ra and IL10Rb sdAbs were generated in substantial accordance with the teaching of the Examples. IL10R binding molecules corresponding to sequence ID NOS: 256-304 are examples of the “forward configuration” or “forward orientation.”. IL10R binding molecules corresponding to sequence ID NOS: 305-353 are examples of compounds of the “reverse orientation” or “reverse configuration”. These “forward orientation” and “revese orientation” molecules were evaluated for activity in accordance with the Examples and the data provided in Table 13. Referring to the binding data presented in Table 13, it can be seen that the orientation of IL10Ra and IL10Rb sdAb binding domains provides for substantial modulation of the IL10 activity in response to the sequential (forward v. reverse) orientation of the IL10Ra sdAb and an IL10Rb sdAbs of the IL10R binding molecules

The CDRs isolated from the VHH molcules prepared above sequences may be used to prepare IL10R binding molecules of both the forward and reverse configuations. In some embodiments, the disclosure provides an IL10R binding molecule of the “forward” configuration wherein the IL10R binding molecule comprises a polyptide from amino to carboxy terminus:
(a) an IL10Ra sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 1, 4, 7, 10, 13, 16, 19, 22, 25, 28, 31, 34, 37, 40, 43, 46, 49, and 52;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 2, 5, 8, 11, 14, 17, 20, 23, 26, 29, 32, 35, 38, 41, 44, 47, 50 and 53; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 3, 6, 9, 12, 15, 18, 21, 24, 27, 30, 33, 36, 39, 42, 45, 48, 51 and 54.
(b) polypeptide linker from 1-50 amino acids, alternatively 1-40 amino acids, alternatively 1-30 amino acids, alternatively 1-20 amino acids, alternatively 1-15 amino acids, alternatively 1-10 amino acids, alternatively 1-8 amino acids, alternatively 1-6 amino acids, alternatively 1-4 amino acids; and
(c) an IL10Rb sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148 and 151;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149 and 152; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150 and 153.

In some embodiments, IL10R binding molecule of the “forward” configuration wherein the IL10R binding molecule comprises a polyptide from amino to carboxy terminus:
(a) an IL10Ra sdAb of comprising an amino acid sequence having at least 90% (e.g., 91%, 92%, 93%, 94%, 95%, 96%, 97%, 98%, 99%, or 100%) sequence identity to a sequence of any one the of SEQ ID NOS: 154-171;
(b) polypeptide linker from 1-50 amino acids, alternatively 1-40 amino acids, alternatively 1-30 amino acids, alternatively 1-20 amino acids, alternatively 1-15 amino acids, alternatively 1-10 amino acids, alternatively 1-8 amino acids, alternatively 1-6 amino acids, alternatively 1-4 amino acids; and
(c) and IL10Rb sdAb comprises a sequence having at least 90% (e.g., 91%, 92%, 93%, 94%, 95%, 96%, 97%, 98%, 99%, or 100%) sequence identity to a sequence of any one of SEQ ID NOS: 172-204.

In some embodiments, and IL10R molecule of the “forward” configuration wherein the IL10R binding molecule comprises a polyptide having at 90% (e.g., 91%, 92%, 93%, 94%, 95%, 96%, 97%, 98%, 99%, or 100%) sequence identity to a sequence of any one the of SEQ ID NOS: 256-304.

In some embodiments, the disclosure provides an IL10R binding molecule of the “reverse” configuration wherein the IL10R binding molecule comprises a polyptide from amino to carboxy terminus:
(a) an IL10Rb sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148 and 151;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149 and 152; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150 and 153.
(b) polypeptide linker from 1-50 amino acids, alternatively 1-40 amino acids, alternatively 1-30 amino acids, alternatively 1-20 amino acids, alternatively 1-15 amino acids, alternatively 1-10 amino acids, alternatively 1-8 amino acids, alternatively 1-6 amino acids, alternatively 1-4 amino acids; and
(c) an IL10Ra sdAb comprising:
(d) a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 1, 4, 7, 10, 13, 16, 19, 22, 25, 28, 31, 34, 37, 40, 43, 46, 49, and 52;
(e) a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 2, 5, 8, 11, 14, 17, 20, 23, 26, 29, 32, 35, 38, 41, 44, 47, 50 and 53; and
(f) a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 3, 6, 9, 12, 15, 18, 21, 24, 27, 30, 33, 36, 39, 42, 45, 48, 51 and 54.

In some embodiments, the disclosure provides an IL10R binding molecule of the “reverse” configuration wherein the IL10R binding molecule comprises a polyptide from amino to carboxy terminus:
(a) and IL10Rb sdAb comprising a sequence having at least 90% (e.g., 91%, 92%, 93%, 94%, 95%, 96%, 97%, 98%, 99%, or 100%) sequence identity to a sequence of any one of SEQ ID NOS: 172-204;
(b) a polypeptide linker from 1-50 amino acids, alternatively 1-40 amino acids, alternatively 1-30 amino acids, alternatively 1-20 amino acids, alternatively 1-15 amino acids, alternatively 1-10 amino acids, alternatively 1-8 amino acids, alternatively 1-6 amino acids, alternatively 1-4 amino acids; and
(c) an IL10Ra sdAb of comprising an amino acid sequence having at least 90% (e.g., 91%, 92%, 93%, 94%, 95%, 96%, 97%, 98%, 99%, or 100%) sequence identity to a sequence of any one the of SEQ ID NOS: 154-171.

In some embodiments, the IL10R binding molecules is a polypeptide having at least 90% (e.g., 91%, 92%, 93%, 94%, 95%, 96%, 97%, 98%, 99%, or 100%) sequence identity to an IL10R binding molecule of formula #1 wherein IL10VHH #1 and IL10VHH #2 are correspond the SEQ ID of a row (e.g. IL10VHH #1 is SEQ ID NO: 165 and IL10VHH #2 is SEQ ID NO: 198, etc.) of the following table:

TABLE 20

| IL10VHH#1 SEQ ID | IL10VHH#2 SEQ ID |
|---|---|
| 165 | 198 |
| 167 | 197 |
| 170 | 187 |
| 183 | 161 |
| 162 | 172 |
| 172 | 162 |
| 161 | 187 |
| 161 | 197 |
| 162 | 197 |
| 161 | 186 |
| 163 | 174 |
| 187 | 163 |
| 161 | 183 |
| 159 | 198 |
| 159 | 187 |
| 162 | 187 |
| 170 | 186 |
| 165 | 187 |
| 161 | 198 |
| 162 | 198 |
| 170 | 197 |
| 167 | 172 |
| 183 | 159 |
| 174 | 163 |
| 186 | 162 |

### Evaluation of the Effects of Linker Length on IL10R Binding Molecule Activity

As discussed in the present disclosure, the activity of the IL10R dimeric VHH IL10R binding molecules of the present disclosure may be modulated by varying the length of the linker between the first and second IL10 VHH monomers. To illustrate the effects of variations of variations in linker length, a series of IL10R binding polypeptides of the formula [#1], in each case b=1 and L2=Ala-Ser; c=1; and CP=his×6 (SEQ ID NO: 532), were prepared containing variations in IL10VHH #2 while holding IL10VHH #1 sequence constant as DR241 (SEQ ID NO 170), the in accordance with the following Table 26:

TABLE 26
IL10Ra/IL10Rb Binding Molecules With Varying Linker Length

| Dimer | | IL10VHH#1 | | L1ₐ | | | IL10VHH#2 | |
|---|---|---|---|---|---|---|---|---|
| | SEQ | VHH | SEQ | | | SEQ | VHH | SEQ |
| Name | ID | Name | ID | a= | L1 | ID | Name | ID |
| DR890 | 489 | DR241 | 170 | 0 | — | n/a | DR244 | 174 |
| DR891 | 490 | DR241 | 170 | 1 | GS | n/a | DR244 | 174 |
| DR519 | 300 | DR241 | 170 | 1 | 4x | 484 | DR244 | 174 |
| DR892 | 491 | DR241 | 170 | 1 | 6x | 470 | DR244 | 174 |
| DR893 | 492 | DR241 | 170 | 1 | 8x | 478 | DR244 | 174 |
| DR894 | 493 | DR241 | 170 | 1 | 10x | 472 | DR244 | 174 |
| DR787 | 494 | DR241 | 170 | 1 | 2x | n/a | DR246 | 187 |
| DR521 | 302 | DR241 | 170 | 1 | 4x | 484 | DR246 | 187 |
| DR896 | 494 | DR241 | 170 | 1 | GS | n/a | DR247 | 197 |
| DR522 | 303 | DR241 | 170 | 1 | 4x | 484 | DR247 | 197 |
| DR897 | 495 | DR241 | 170 | 1 | 6x | 470 | DR247 | 197 |
| DR898 | 496 | DR241 | 170 | 1 | 8x | 478 | DR247 | 197 |
| DR899 | 497 | DR241 | 170 | 1 | 10x | 472 | DR247 | 197 |

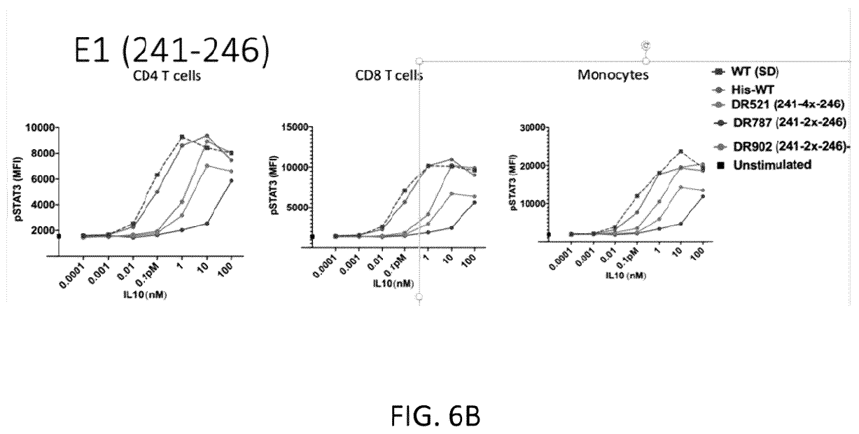

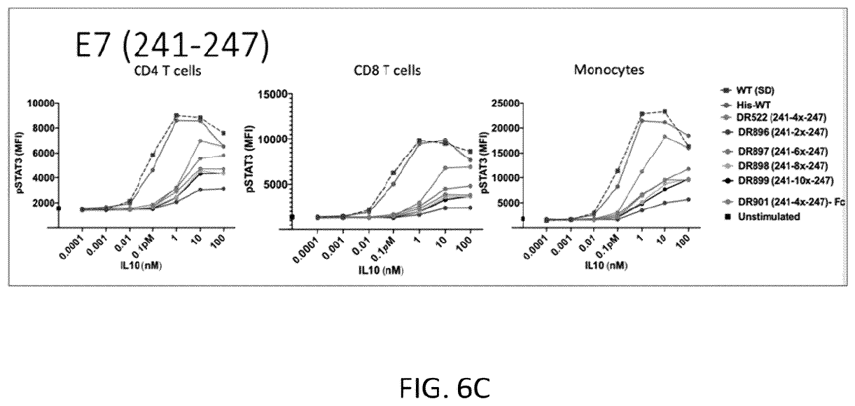

The level of pSTAT3 indued in response to the IL10R binding molecules corresponding to SEQ ID NOS: 489, 490, 300, 491, 492, 493, 494, 302, 494, 303, 495, 496 and 497 were evaluated for activity on CD4+ T cells, CD8+ T cells and monocytes using wild-type human IL10, a wild-type IL10 comprising a C-terminal His6 (SEQ ID NO: 532) chelating peptide and unstimilated as controls. The protocol for the assay and methodology involved is presented in FIG. 5 of the attached drawings. The results of these tests is provided in FIG. 6 of the attached drawings. To clarify the effect of linker length alone, in FIG. 6A-6C represent the condition where the IL10Ra sdAb and IL10Rb sdAb were held constant and only the length of the linker is varied the term 2×, 4×, etc. refers to the number of amino acids in the linker and the sequence of the linker is provided in Table 16. As illustrated by the data presented in FIG. 6, the variation of the linker length alone (maintain the sdAbs and other components constant as is reflected in FIGS. 6A-6C) may be used to modulate not only the level of activity of the IL10R binding molecule with repect to a given cell type (e.g. CD4+, CD8+, or monocyte), the variation in linker length may be used to reflect the modulating the activity of one cell type to another. As illustrated by the data provided variation of the distance between the IL10Ra and IL10Rb sdAbs, in some instances varying the length of the linker molecules, can provide modulation of the level of IL10 activity in a cell as measured by the levels of pSTAT3 activity.

### Fc Conjugates

As discussed in the present disclosure, in some embodiments, the IL10R binding molecule may be conjugated to a subunit of an Fc domain as illustrated in FIG. 2B, and represented by the formula #2:

H2N-(IL10 VHH #1)-(L1)ₐ-(IL10 VHH #1)-(L2)_{b}-(Fc monomer)_{c}-COOH [#2]

A representative series of IL10R binding polypeptides of the formula [#1], in each case a=1, L1=G3S (SEQ ID NO: 484), b=1 and L2=Ala-Ser; c=1; and Fc monomer is a polypeptide of SEQ ID NO:502, were prepared containing variations in IL10VHH #2 while holding IL10VHH #1 sequence constant as DR241 (SEQ ID NO 170), the in accordance with the following Table 27. These features and details of the IL10Ra and IL10Rb sdAbs used in their construction are summarized in the following Table 27:

TABLE 27
IL10Ra/IL10Rb Binding Molecules With Varying Linker Length

| Dimer | | IL10VHH#1 | | IL10VHH#2 | | L2 and Fc Monomer | |
|---|---|---|---|---|---|---|---|
| | SEQ | VHH | SEQ | VHH | SEQ | | Fc |
| Name | ID | Name | ID | Name | ID | L2 | Monomer |
| DR900 | 499 | DR241 | 170 | DR244 | 174 | AS | SEQ ID |
| | | | | | | | NO: 502 |
| DR902 | 500 | DR241 | 170 | DR246 | 187 | AS | SEQ ID |
| | | | | | | | NO: 502 |
| DR901 | 501 | DR241 | 170 | DR247 | 197 | AS | SEQ ID |
| | | | | | | | NO: 502 |

Data relating to the activity of each of these molecules at varying conentrations in CD4+, CD8+ and monocytes is also provided in FIG. 6. It should be noted that each of these Fc fusions was included relative to the non-Fc fused molecules having the same IL10Ra and IL10Rb sdAbs in the same orientation. Thus the data provided in FIG. 6 also may be used to evaluate the effect on IL10 activity of the IL10R binding molecule by the addition of the Fc domain. From the data provided, the Fc fusion fusion molecule containg the same IL10Ra and Il10Rb sdAbs in the same orientation (forward or reverse) and compared with the molecules having the same linker (4× als referred to as G3S (SEQ ID NO: 484), see Table 16) linker, the Fc fusions demonstrated comparable, in some instances enhanced activity relative to the non-Fc variant. This data demonstrates that the IL10R binding molecules of the present disclosure may be conjugated to an Fc domain to provide extended lifetime in vivo without and providing a level of IL10 activity in various cell types comparable to the non-Fc fused molecule.

### Construction of IL10R Binding Molecules for Further Evaluation

Based on the foregoing experiments, a selected subset of IL10R binding molecules variants based on DR521 (SEQ ID NO: 304) were selected for further evaluation. The molecules used in these experiments may be represented by the formula [#1], wherein in each case a=1, L1=G3S (SEQ ID NO: 484) (SEQ ID NOr, b=1 and L2=Ala-Ser; CP=His×6 (SEQ ID NO: 532), the IL10 VHH #1 is the IL10Ra VHH DR241 (SEQ ID NO 170) and the IL10 VHH #2 is the IL10Rb VHH DR 246 (SEQ ID NO:187). The polypeptide of SEQ ID NO: 170 is also referred to herein as DR241 and the polypeptide of SEQ ID NO:187 is also referred to herein as DR246.

### DR521 (DR241-G3S-DR246-ASH6):

In one embodiment, exemplary polypeptide prepared for evaluation is a polypeptide of formula [#1], wherein in each case a=1, L1=G3S (SEQ ID NO: 484)-Ser, b=1 and L2=Ala-Ser; CP=His×6 (SEQ ID NO: 532), the IL10 VHH #1 is the IL10Ra VHH DR241 (SEQ ID NO 170) and the IL10 VHH #2 is the IL10Rb VHH DR 246 (SEQ ID NO:187): a=1 and L1 is a G3S linker (SEQ ID NO: 484); b=1 and L2 has the sequence Ala-Ser; and c=1 wherein CP is a histidine polypeptide hexamer (SEQ ID NO: 532), which polypeptide has the amino acid sequence:

(SEQ ID NO: 302)
QVQLQESGGGSVQAGGSLRLSCAASGYSNCSYDMTWYRQAPGKEREFV
SAIHSDGSTRYADSVKGRFFISQDNAKNTVYLQMNSLKPEDTAMYYCK
TDPLHCRAHGGSWYSVRANYWGQGTQVTVSSGGGSQVQLQESGGGSVE
AGGSLRLSCAASGYTHSSYCMGWFRQAPGKEREGVAAIDVDGSTTYAD
SVKGRFTISKDNAKNTLYLQMNSLKPEDTGMYYCAAEFADCSSNYFLP
PGAVRYWGQGTQVTVSSASHHHHHH

The polypeptide of SEQ ID NO:302 is also referred to herein as DR521.

### DR838 (Q1E N29Q DR521):

A polypeptide variant of DR521 (SEQ ID NO:302) SEQ ID NO:302 which incorporates amino acid substitutions 1QE and N29Q was prepared was has the amino acid sequence:

(SEQ ID NO: 486)
EVQLQESGGGSVQAGGSLRLSCAASGYSQCSYDMTWYRQAPGKEREFVS
AIHSDGSTRYADSVKGRFFISQDNAKNTVYLQMNSLKPEDTAMYYCKTD
PLHCRAHGGSWYSVRANYWGQGTQVTVSGGGSQVQLQESGGGSVEAGGS
LRLSCAASGYTHSSYCMGWFRQAPGKEREGVAAIDVDGSTTYADSVKGR
FTISKDNAKNTLYLQMNSLKPEDTGMYYCAAEFADCSSNYFLPPGAVRY
WGQGTQVTVSSASHHHHHH.

The polypeptide of SEQ ID NO: 486 is also referred to herein as DR838.

As discussed elsewhere, to avoid pyroglutamate formation at the N-terminus which complicates PEGylation by aldehyde chemistry, in the preparation of the DR838 (SEQ ID NO: 486) a Q1E amino acid substation was incorporated to facilitate N-terminal PEGylation. Additionally, an examination of the sequence of the CDRs of DR521 (SEQ ID NO:302) indicates the presence of an N-linked glycosylation motif N-X-S in the sequence at Asn29-Cys30-Ser30. To eliminate such N-linked glycosylation motifs by modifying the sequence of the N-linked glycosylation motif to prevent glycosylation. In the preparation of the DR838 (SEQ ID NO:486) derivative of DR521 (SEQ ID NO:302), this N-linked glycosylation motif was eliminated by substituting the asparagine at position 29 of DR521 with a glutamine residue referred to as an N29Q substitution.

### DR839 Q1E N29D DR521-Ala-Ser-HIS6

An additional polypeptide variant of SEQ ID NO:302 was prepared having an Q1E and N29D amino acid substitutions and has the amino acid sequence:

(SEQ ID NO: 487)
EVQLQESGGGSVQAGGSLRLSCAASGYSDCSYDMTWYRQAPGKEREFVS
AIHSDGSTRYADSVKGRFFISQDNAKNTVYLQMNSLKPEDTAMYYCKTD
PLHCRAHGGSWYSVRANYWGQGTQVTVSSGGGSQVQLQESGGGSVEAGG
SLRLSCAASGYTHSSYCMGWFRQAPGKEREGVAAIDVDGSTTYADSVKG
RFTISKDNAKNTLYLQMNSLKPEDTGMYYCAAEFADCSSNYFLPPGAVR
YWGQGTQVTVSSASHHHHHH.

The polypeptide of SEQ ID NO: 487 is also referred to herein as DR839.

### DR841 (Q1E S31A DR521)

An additional polypeptide variant of SEQ ID NO:302 was prepared having an Q1E and S31A amino acid substitution (to eliminate the Asn29-Cys30-Ser30 N-linked glycosylation motif of DR521) and has the amino acid sequence:

(SEQ ID NO: 488)
EVQLQESGGGSVQAGGSLRLSCAASGYSNCAYDMTWYRQAPGKEREFVS
AIHSDGSTRYADSVKGRFFISQDNAKNTVYLQMNSLKPEDTAMYYCKTD
PLHCRAHGGSWYSVRANYWGQGTQVTVSSGGGSQVQLQESGGGSVEAGG
SLRLSCAASGYTHSSYCMGWFRQAPGKEREGVAAIDVDGSTTYADSVKG
RFTISKDNAKNTLYLQMNSLKPEDTGMYYCAAEFADCSSNYFLPPGAVR
YWGQGTQVTVSSASHHHHHH.

The polypeptide of SEQ ID NO: 488 is also referred to herein as DR841.

Humanization of sdAbs

As previously discussed, the IL10Ra and IL10Rb VHH sdAbs which are useful in the preparation of IL10R binding molecules of the present disclosure may be humanized. To demonstrate the utility of humanized versions of the VHH components of the IL10R binding molecules, humanized versions of DR241 and DR246 were prepared and evaluated for binding to the ECDs of IL10Ra and IL10Rb, respectively by surface plasmon resonance spectroscopy (Biacore®).

When humanizing VHHs with respect to a target, a consideration for the design of the humanized VHH is the distribution of amino acids at each position of the non-human framework which suggest amino acid residues the modification of which may introduce substantial modifications in the secondary and tertiary structure of the protein. The literature suggests that certain framework residues of the canonical llama VHH sequence VH3-66 (INSERT REF/SEQ) include positions V37, G44, L45 and W47 and vernier zone residues R94 and W103 may also be residues which are not readily susceptible to modifiation. To identify amino acids of the camel VHH framework regions which are highly conserved, approximately 1023 VHH sdAb sequences were obtained and a “R Script” to evaluate distributions of amino acids at each position. An amino acid distribution chart was used to identify rare residues at each position. It was determined that certain residues of the camel VHH framework regions are highly conserved.

Based this information, a best-fit human germline sequence VH3—was identified. A sequence alignment of DR241 suggests a 72% identity to best fit human germline sequence VH3-23. Humanized versions of the IL10Ra VHH DR241 (See Table 19). Humanized DR241 constructs show 88% identity to human. Humanized versions of the IL10Rb VHH DR246 (Table 19) were also prepared. A sequence alignment of DR241 suggests that DR246 possess 73% identity to best fit human germline VH3-23. The humanized DR246 (DR1228) construct shows 89% identity to human.

The following Table 19 provides examples of humanized IL10R binding molecules of the present disclosure

TABLE 19
Humanized IL10Ra/Rb VHH Binding Molecules

| | Descrip- | | SEQ |
|---|---|---|---|
| Name | tion | Amino Acid Sequence (CDRs Underlined) | ID NO |
| DR1223 | Humanized | EVQLLESGGGLVQPGGSLRLSCAASGYTQCSYDMTWVR | 503 |
| | DR241 | QAPGKGLEWVSAIHSDGSTRYADSVKGRFTISRDNSKN | |
| | IL10Ra | TLYLQMNSLRAEDTAVYYCKTDPLHCRAHGGSWYSVRA | |
| | | NYWGQGTLVTVSS | |
| DR1224 | Humanized | EVQLLESGGGLVQPGGSLRLSCAASGYTQCSYDMTWVR | 504 |
| | DR241 | QAPGKGLEFVSAIHSDGSTRYADSVKGRFTISRDNSKN | |
| | IL10Ra | TLYLQMNSLRAEDTAVYYCKTDPLHCRAHGGSWYSVRA | |
| | | NYWGQGTLVTVSS | |
| DR1225 | Humanized | EVQLVESGGGLVQPGGSLRLSCAASGYTQCSYDMTWVR | 505 |
| | DR241 | QAPGKGLEWVSAIHSDGSTRYADSVKGRFFISRDNSKN | |
| | IL10Ra | TLYLQMNSLRAEDTAVYYCKTDPLHCRAHGGSWYSVRA | |
| | | NYWGQGTLVTVSS | |
| DR1226 | Humanized | EVQLVESGGGLVQPGGSLRLSCAASGYTQCSYDMTWVR | 506 |
| | DR241 | QAPGKGLEFVSAIHSDGSTRYADSVKGRFFISRDNSKN | |
| | IL10Ra | TLYLQMNSLRAEDTAVYYCKTDPLHCRAHGGSWYSVRA | |
| | | NYWGQGTLVTVSS | |
| DR1227 | Humanized | EVQLLESGGGLVQPGGSLRLSCAASGYTHSSYCMGWVR | 507 |
| | DR246 | QAPGKGLEWVSAIDVDGSTTYADSVKGRFTISRDNSKN | |
| | IL10Rb | TLYLQMNSLRAEDTAVYYCAAEFADCSSNYFLPPGAVR | |
| | | YWGQGTLVTVSS | |
| DR1228 | Humanized | EVQLLESGGGLVQPGGSLRLSCAASGYTHSSYCMGWVR | 508 |
| | DR246 | QAPGKGLEGVSAIDVDGSTTYADSVKGRFTISRDNSKN | |
| | IL10Rb | TLYLQMNSLRAEDTAVYYCAAEFADCSSNYFLPPGAVR | |
| | | YWGQGTLVTVSS | |
| DR1229 | Humanized | EVQLVESGGGLVQPGGSLRLSCAASGYTHSSYCMGWVR | 509 |
| | DR246 | QAPGKGLEWVSAIDVDGSTTYADSVKGRFTISRDNSKN | |
| | IL10Rb | TLYLQMNSLRAEDTAVYYCAAEFADCSSNYFLPPGAVR | |
| | | YWGQGTLVTVSS | |
| DR1230 | Humanized | EVQLVESGGGLVQPGGSLRLSCAASGYTHSSYCMGWVR | 510 |
| | DR246 | QAPGKGLEGVSAIDVDGSTTYADSVKGRFTISRDNSKN | |
| | IL10Rb | TLYLQMNSLRAEDTAVYYCAAEFADCSSNYFLPPGAVR | |
| | | YWGQGTLVTVSS | |
| DR1308 | Humanized | EVQLVESGGGLVQPGGSLRLSCAASGYTQCSYDMTWVR | 511 |
| | DR241 | QAPGKGREFVSAIHSDGSTRYADSVKGRFFISRDNSKN | |
| | IL10Ra | TLYLQMNSLRAEDTAVYYCKTDPLHCRAHGGSWYSVRA | |
| | | NYWGQGTLVTVSS | |
| DR1309 | Humanized | EVQLQESGGGSVQAGGSLRLSCAASGYSQCSYDMTWYR | 512 |
| | DR241 | QAPGKEREFVSAIHSDGSTRYADSVKGRFFISQDNAKN | |
| | IL10Ra | TVYLQMNSLKPEDTAMYYCKTDPLHCRAHGGSWYSVRA | |
| | | NYWGQGTQVTVSS | |

Nucleic acid sequences encoding the foregoing humanized sdAb are provided in Table 30.

In some embodiments, the present invention provides sdAbs having at least 90% (e.g., 91%, 92%, 93%, 94%, 95%, 96%, 97%, 98%, 99%, or 100%) sequence identity to the sequence of any one of the IL10Ra and IL10Rb sdAbs of Table 19. In some embodiments, the present invention provides sdAbs having substantially identical of any one of the sdAbs Table 19. In some embodiments, the present invention provides sdAbs having identical of any one of the sdAbs Table 19.

In the following series of experiments, the above referenced polypeptides were expressed recombinantly by transfection of a host cell with a vector comprising a synthetic nucleic acid sequences encoding DR521 and DR838, the synthetic nucleic acid sequence incorporating a 5′ nucleic acid sequence encoding IgH signal peptide of the amino acid sequence.

Synthetic nucleic acid sequences encoding the DR1223, DR1224; DR1225, DR1226; DR1227; DR1228; DR1229 and DR1230 are provided in Table 31 urther comprising N-terminal signal peptide and carboxy terminal IgG4 hinge (SEQ ID NO: 485) and monomeric Fc domain (SEQ ID NO: 504) were prepared and cloned in pExSyn and transfected into expi293 cells. As provided in more detail in the Examples, the molecules were evaluated for binding to their respective targets (IL10Ra or IL10Rb) by SPR (Biacore). The supernatants from the cell culture were evaluated for binding to the ECDs of IL10Ra and IL10Rb respectively. In short, the Fc conjugated humanized VHHs were immobilized on a Protein A chip and the ECDs of the respective IL10 receptor subunits flowed in the mobile phase. The results of this study indicated that the humanized VHH sequences effectively retained binding to their respective targets. The foregoing humanized IL10Ra and IL10Rb VHHs may be used in the construction of humanized dimeric IL10R binding molecules of the present disclosure.

TABLE 28
Humanized versions of DR246 binding to IL10Rb-
his (Sino Biological, Catalog#10945)

| | | | | | | Calc. | |
|---|---|---|---|---|---|---|---|
| | k_{ON} | k_{OFF} | Affinity | Rmax | Load | Rmax | Surface |
| Analyte | (1/Ms) | (1/s) | (nM) | (RU) | (RU) | (RU) | Activity |
| DR1227 | 4.6E+05 | 5.6E−03 | 12.3 | 68.3 | 327 | 497 | 14% |
| DR1228 | 1.3E+05 | 9.2E−04 | 7.0 | 112.4 | 328 | 499 | 23% |
| DR1229 | 7.0E+05 | 9.6E−03 | 13.8 | 68.6 | 330 | 502 | 14% |
| DR1230 | 1.4E+05 | 9.8E−04 | 7.1 | 121.8 | 331 | 504 | 24% |

### Humanized IL10R Binding Molecules:

The present disclosure further provides IL10R binding molecules comprising humanized IL10Ra and/or IL10Rb sdAbs. The term “humanized IL10R binding molecule” refers to an IL10R binding molecule comprising a humanized IL10Ra and/or IL10Rb sdAbs.

The following Table 29 provides amino acid sequences of humanized IL10R binding molecules comprising IL10Ra and IL10Rb sdAbs and indicateing the CDRs as underlined.

TABLE 29
Humanized IL10R Binding Molecules

| | | SEQ |
|---|---|---|
| Name | Amino Acid Sequence | ID NO |
| DR1511 | EVQLVESGGGLVKPGGSLRLSCAASGYSNCAYDMTW | 513 |
| | YRQAPGKGLEWVSAIHSDGSTRYADSVKGRFFISRDN | |
| | AKNSLYLQMNSLRAEDTAVYYCKTDPLHCRAHGGS | |
| | WYSVRANYWGQGTLVTVSSGGGSEVQLVESGGGLV | |
| | QPGGSLRLSCAASGYTHSSYCMGWVRQAPGKGLEGV | |
| | SAIDVDGSTTYADSVKGRFTISRDNSKNTLYLQMNSL | |
| | RAEDTAVYYCAAEFADCSSNYFLPPGAVRYWGQGTL | |
| | VTVSSASHHHHHH | |
| DR1512 | EVQLVESGGGLVQPGGSLRLSCAASGYSNCAYDMTW | 514 |
| | YRQAPGKGLEWVSAIHSDGSTRYADSVKGRFFISRDN | |
| | SKNTLYLQMNSLRAEDTAVYYCKTDPLHCRAHGGS | |
| | WYSVRANYWGQGTLVTVSSGGGSEVQLVESGGGLV | |
| | QPGGSLRLSCAASGYTHSSYCMGWVRQAPGKGLEGV | |
| | SAIDVDGSTTYADSVKGRFTISRDNSKNTLYLQMNSL | |
| | RAEDTAVYYCAAEFADCSSNYFLPPGAVRYWGQGTL | |
| | VTVSSASHHHHHH | |
| DR1513 | EVQLLESGGGLVQPGGSLRLSCAASGYSNCAYDMTW | 515 |
| | YRQAPGKGLEWVSAIHSDGSTRYADSVKGRFFISRDN | |
| | SKNTLYLQMNSLRAEDTAVYYCKTDPLHCRAHGGS | |
| | WYSVRANYWGQGTLVTVSSGGGSEVQLVESGGGLV | |
| | QPGGSLRLSCAASGYTHSSYCMGWVRQAPGKGLEGV | |
| | SAIDVDGSTTYADSVKGRFTISRDNSKNTLYLQMNSL | |
| | RAEDTAVYYCAAEFADCSSNYFLPPGAVRYWGQGTL | |
| | VTVSSASHHHHHH | |
| DR1514 | EVQLVESGGGLVKPGGSLRLSCAASGYSNCAYDMTW | 516 |
| | YRQAPGKGLEFVSAIHSDGSTRYADSVKGRFFISRDN | |
| | AKNSLYLQMNSLRAEDTAVYYCKTDPLHCRAHGGS | |
| | WYSVRANYWGQGTLVTVSSGGGSEVQLVESGGGLV | |
| | QPGGSLRLSCAASGYTHSSYCMGWVRQAPGKGLEGV | |
| | SAIDVDGSTTYADSVKGRFTISRDNSKNTLYLQMNSL | |
| | RAEDTAVYYCAAEFADCSSNYFLPPGAVRYWGQGTL | |
| | VTVSSASHHHHHH | |
| DR1515 | EVQLVESGGGLVQPGGSLRLSCAASGYSNCAYDMTW | 517 |
| | YRQAPGKGLEFVSAIHSDGSTRYADSVKGRFFISRDNS | |
| | KNTLYLQMNSLRAEDTAVYYCKTDPLHCRAHGGSW | |
| | YSVRANYWGQGTLVTVSSGGGSEVQLVESGGGLVQP | |
| | GGSLRLSCAASGYTHSSYCMGWVRQAPGKGLEGVSA | |
| | IDVDGSTTYADSVKGRFTISRDNSKNTLYLQMNSLRA | |
| | EDTAVYYCAAEFADCSSNYFLPPGAVRYWGQGTLVT | |
| | VSSASHHHHHH | |
| DR1516 | EVQLLESGGGLVQPGGSLRLSCAASGYSNCAYDMTW | 518 |
| | YRQAPGKGLEFVSAIHSDGSTRYADSVKGRFFISRDNS | |
| | KNTLYLQMNSLRAEDTAVYYCKTDPLHCRAHGGSW | |
| | YSVRANYWGQGTLVTVSSGGGSEVQLVESGGGLVQP | |
| | GGSLRLSCAASGYTHSSYCMGWVRQAPGKGLEGVSA | |
| | IDVDGSTTYADSVKGRFTISRDNSKNTLYLQMNSLRA | |
| | EDTAVYYCAAEFADCSSNYFLPPGAVRYWGQGTLVT | |
| | VSSASHHHHHH | |
| DR1517 | EVQLVESGGGLVKPGGSLRLSCAASGYSNCAYDMTW | 519 |
| | YRQAPGKGREWVSAIHSDGSTRYADSVKGRFFISRDN | |
| | AKNSLYLQMNSLRAEDTAVYYCKTDPLHCRAHGGS | |
| | WYSVRANYWGQGTLVTVSSGGGSEVQLVESGGGLV | |
| | QPGGSLRLSCAASGYTHSSYCMGWVRQAPGKGLEGV | |
| | SAIDVDGSTTYADSVKGRFTISRDNSKNTLYLQMNSL | |
| | RAEDTAVYYCAAEFADCSSNYFLPPGAVRYWGQGTL | |
| | VTVSSASHHHHHH | |
| DR1518 | EVQLVESGGGLVQPGGSLRLSCAASGYSNCAYDMTW | 520 |
| | YRQAPGKGREWVSAIHSDGSTRYADSVKGRFFISRDN | |
| | SKNTLYLQMNSLRAEDTAVYYCKTDPLHCRAHGGS | |
| | WYSVRANYWGQGTLVTVSSGGGSEVQLVESGGGLV | |
| | QPGGSLRLSCAASGYTHSSYCMGWVRQAPGKGLEGV | |
| | SAIDVDGSTTYADSVKGRFTISRDNSKNTLYLQMNSL | |
| | RAEDTAVYYCAAEFADCSSNYFLPPGAVRYWGQGTL | |
| | VTVSSASHHHHHH | |
| DR1519 | EVQLLESGGGLVQPGGSLRLSCAASGYSNCAYDMTW | 521 |
| | YRQAPGKGREWVSAIHSDGSTRYADSVKGRFFISRDN | |
| | SKNTLYLQMNSLRAEDTAVYYCKTDPLHCRAHGGS | |
| | WYSVRANYWGQGTLVTVSSGGGSEVQLVESGGGLV | |
| | QPGGSLRLSCAASGYTHSSYCMGWVRQAPGKGLEGV | |
| | SAIDVDGSTTYADSVKGRFTISRDNSKNTLYLQMNSL | |
| | RAEDTAVYYCAAEFADCSSNYFLPPGAVRYWGQGTL | |
| | VTVSSASHHHHHH | |
| DR1520 | EVQLVESGGGLVKPGGSLRLSCAASGYSNCAYDMTW | 522 |
| | YRQAPGKELEWVSAIHSDGSTRYADSVKGRFFISRDN | |
| | AKNSLYLQMNSLRAEDTAVYYCKTDPLHCRAHGGS | |
| | WYSVRANYWGQGTLVTVSSGGGSEVQLVESGGGLV | |
| | QPGGSLRLSCAASGYTHSSYCMGWVRQAPGKGLEGV | |
| | SAIDVDGSTTYADSVKGRFTISRDNSKNTLYLQMNSL | |
| | RAEDTAVYYCAAEFADCSSNYFLPPGAVRYWGQGTL | |
| | VTVSSASHHHHHH | |
| DR1521 | EVQLVESGGGLVQPGGSLRLSCAASGYSNCAYDMTW | 523 |
| | YRQAPGKELEWVSAIHSDGSTRYADSVKGRFFISRDN | |
| | SKNTLYLQMNSLRAEDTAVYYCKTDPLHCRAHGGS | |
| | WYSVRANYWGQGTLVTVSSGGGSEVQLVESGGGLV | |
| | QPGGSLRLSCAASGYTHSSYCMGWVRQAPGKGLEGV | |
| | SAIDVDGSTTYADSVKGRFTISRDNSKNTLYLQMNSL | |
| | RAEDTAVYYCAAEFADCSSNYFLPPGAVRYWGQGTL | |
| | VTVSSASHHHHHH | |
| DR1522 | EVQLLESGGGLVQPGGSLRLSCAASGYSNCAYDMTW | 561 |
| | YRQAPGKELEWVSAIHSDGSTRYADSVKGRFFISRDN | |
| | SKNTLYLQMNSLRAEDTAVYYCKTDPLHCRAHGGS | |
| | WYSVRANYWGQGTLVTVSSGGGSEVQLVESGGGLV | |
| | QPGGSLRLSCAASGYTHSSYCMGWVRQAPGKGLEGV | |
| | SAIDVDGSTTYADSVKGRFTISRDNSKNTLYLQMNSL | |
| | RAEDTAVYYCAAEFADCSSNYFLPPGAVRYWGQGTL | |
| | VTVSSASHHHHHH | |
| DR1523 | EVQLVESGGGLVKPGGSLRLSCAASGYSNCAYDMTW | 524 |
| | YRQAPGKEREFVSAIHSDGSTRYADSVKGRFFISRDNA | |
| | KNSLYLQMNSLRAEDTAVYYCKTDPLHCRAHGGSW | |
| | YSVRANYWGQGTLVTVSSGGGSEVQLVESGGGLVQP | |
| | GGSLRLSCAASGYTHSSYCMGWVRQAPGKGLEGVSA | |
| | IDVDGSTTYADSVKGRFTISRDNSKNTLYLQMNSLRA | |
| | EDTAVYYCAAEFADCSSNYFLPPGAVRYWGQGTLVT | |
| | VSSASHHHHHH | |
| DR1524 | EVQLVESGGGLVQPGGSLRLSCAASGYSNCAYDMTW | 525 |
| | YRQAPGKEREFVSAIHSDGSTRYADSVKGRFFISRDNS | |
| | KNTLYLQMNSLRAEDTAVYYCKTDPLHCRAHGGSW | |
| | YSVRANYWGQGTLVTVSSGGGSEVQLVESGGGLVQP | |
| | GGSLRLSCAASGYTHSSYCMGWVRQAPGKGLEGVSA | |
| | IDVDGSTTYADSVKGRFTISRDNSKNTLYLQMNSLRA | |
| | EDTAVYYCAAEFADCSSNYFLPPGAVRYWGQGTLVT | |
| | VSSASHHHHHH | |
| DR1525 | EVQLLESGGGLVQPGGSLRLSCAASGYSNCAYDMTW | 526 |
| | YRQAPGKEREFVSAIHSDGSTRYADSVKGRFFISRDNS | |
| | KNTLYLQMNSLRAEDTAVYYCKTDPLHCRAHGGSW | |
| | YSVRANYWGQGTLVTVSSGGGSEVQLVESGGGLVQP | |
| | GGSLRLSCAASGYTHSSYCMGWVRQAPGKGLEGVSA | |
| | IDVDGSTTYADSVKGRFTISRDNSKNTLYLQMNSLRA | |
| | EDTAVYYCAAEFADCSSNYFLPPGAVRYWGQGTLVT | |
| | VSSASHHHHHH | |
| DR1328 | EVQLVESGGGLVQPGGSLRLSCAASGYTQCSYDMTW | 527 |
| | VRQAPGKGLEFVSAIHSDGSTRYADSVKGRFFISRDNS | |
| | KNTLYLQMNSLRAEDTAVYYCKTDPLHCRAHGGSW | |
| | YSVRANYWGQGTLVTVSSGGGSEVQLVESGGGLVQP | |
| | GGSLRLSCAASGYTHSSYCMGWVRQAPGKGLEWVS | |
| | AIDVDGSTTYADSVKGRFTISRDNSKNTLYLQMNSLR | |
| | AEDTAVYYCAAEFADCSSNYFLPPGAVRYWGQGTLV | |
| | TVSSASHHHHHH | |
| DR1329 | EVQLVESGGGLVQPGGSLRLSCAASGYTQCSYDMTW | 528 |
| | VRQAPGKGLEFVSAIHSDGSTRYADSVKGRFFISRDNS | |
| | KNTLYLQMNSLRAEDTAVYYCKTDPLHCRAHGGSW | |
| | YSVRANYWGQGTLVTVSSGGGSEVQLVESGGGLVQP | |
| | GGSLRLSCAASGYTHSSYCMGWVRQAPGKGLEGVSA | |
| | IDVDGSTTYADSVKGRFTISRDNSKNTLYLQMNSLRA | |
| | EDTAVYYCAAEFADCSSNYFLPPGAVRYWGQGTLVT | |
| | VSSASHHHHHH | |
| DR1330 | EVQLVESGGGLVQPGGSLRLSCAASGYTQCSYDMTW | 529 |
| | YRQAPGKGLEFVSAIHSDGSTRYADSVKGRFFISRDNS | |
| | KNTLYLQMNSLRAEDTAVYYCKTDPLHCRAHGGSW | |
| | YSVRANYWGQGTLVTVSSGGGSEVQLVESGGGLVQP | |
| | GGSLRLSCAASGYTHSSYCMGWVRQAPGKGLEWVS | |
| | AIDVDGSTTYADSVKGRFTISRDNSKNTLYLQMNSLR | |
| | AEDTAVYYCAAEFADCSSNYFLPPGAVRYWGQGTLV | |
| | TVSSASHHHHHH | |
| DR1331 | EVQLVESGGGLVQPGGSLRLSCAASGYTQCSYDMTW | 530 |
| | YRQAPGKGLEFVSAIHSDGSTRYADSVKGRFFISRDNS | |
| | KNTLYLQMNSLRAEDTAVYYCKTDPLHCRAHGGSW | |
| | YSVRANYWGQGTLVTVSSGGGSEVQLVESGGGLVQP | |
| | GGSLRLSCAASGYTHSSYCMGWVRQAPGKGLEGVSA | |
| | IDVDGSTTYADSVKGRFTISRDNSKNTLYLQMNSLRA | |
| | EDTAVYYCAAEFADCSSNYFLPPGAVRYWGQGTLVT | |
| | VSSASHHHHHH | |

In some embodiments, the present invention provides IL10R binding molecules having at least 90% (e.g., 91%, 92%, 93%, 94%, 95%, 96%, 97%, 98%, 99%, or 100%) sequence identity to the sequence of any one of the IL10R binding molecules of Table 29. In some embodiments, the present invention provides IL10R binding molecules substantially identical of any one of the IL10R binding molecules of Table 29. In some embodiments, the present invention provides IL10R binding molecules identical to a sequence of any one of the IL10R binding molecules of Table 29.

Modifications to the Binding Molecule and sdAb Components

### Chelating Peptides

In one embodiment, the present disclosure provides a IL10Rb1 binding molecule comprising one or more transition metal chelating polypeptide sequences known as chelating papetides. A chelating peptide is a polypeptide of the formula:

(His)ₐ-(AA)_{b}-(His)_{c}

wherein “His” is the amino acid histidine; “AA” is an amino acid other than proline; is a histidine residuea=an integer from 0 to 10; b=an integer from 0 to 4; c=an integer from 0-10; and random, block and alternating copolymers thereof. In some embodiments, the chelating peptide has and amino acid sequence selected from the group consisting of: SEQ ID NOS: 507-521. The incorporation of such a transition metal chelating domain facilitates purification immobilized metal affinity chromatography (IMAC) as described in Smith, et al. U.S. Pat. No. 4,569,794 issued Feb. 11, 1986. Examples of transition metal chelating polypeptides useful in the practice of the present IL12RB1 binding molecule are described in Smith, et al. supra and Dobeli, et al. U.S. Pat. No. 5,320,663 issued May 10, 1995, the entire teachings of which are hereby incorporated by reference. Particular transition metal chelating polypeptides useful in the practice of the present IL12RB1 binding molecule are polypeptides comprising 3-6 contiguous histidine residues (SEQ ID NO: 569) such as a six-histidine (His) 6 peptide (SEQ ID NO: 532) and are frequently referred to in the art as “His-tags.” In addition to providing a purification “handle” for the recombinant proteins or to facilitate immobilization on SPR sensor chips, such the conjugation of the hIL12RB1 binding molecule to a chelating peptide facilitates the targeted delivery to IL12RB1 expressing cells of transition metal ions as kinetically inert or kinetically labile complexes in substantial accordance with the teaching of Anderson, et al., (U.S. Pat. No. 5,439,829 issued Aug. 8, 1995 and Hale, J. E (1996) Analytical Biochemistry 231 (1): 46-49. Regarding the “kinetically inert complex”, the term “inert” refers to the degree of lability which to the ability of a particular complexed ion to engage in reactions that result in replacing one or more ligands in its coordination sphere by others. In an aqueous environment, the unoccupied coordination positions of the transition metal are occupied by water molecules. These water molecules must be displaced by the chelating peptide or organic chelating agent in order to form [transition metal: chelating peptide] complex. When such reactions occur rapidly, the reaction is termed “labile”. However, where such reactions occur very slowly, the complex is said to be kinetically “inert”. Kinetic lability or inertness is related to the reaction rate and are not to be confused with thermodynamic stability or instability. Cotton and Wilkinson illustrate this distinction:

A simple example of this [labile vs. stable] distinction is provided by the [Co(NH3)6]3+ ion which will persist for days in an acid medium because of its kinetic inertness or lack of lability despite the fact that it is thermodynamically unstable, as the following equilibrium constant shows:

[Co(NH₃)₆]³⁺+6H₃O⁺═[Co(H₂O)₆]³⁺+6NH₄⁺ K=10²⁵

In contrast, the stability of [Ni(CN)4]⁻² is extremely high:

[Ni(CN)₄]²⁻═Ni²⁺+4CN⁻ K=10⁻²²
but the rate of exchange of CN⁻ ions with isotopically labelled cyanide ion added to the solution is immeasurably fast by ordinary techniques.
   Advanced Inorganic Chemistry, Cotton, F. A. and Wilkinson, G. (1972) 3rd ed. Interscience Publishers, p. 652. In some embodiments, the transition metal ion is a reporter molecule such as a fluorescent compound or radioactive agent, including as radiological imaging or therapeutic agents.

In some embodiments, the chelating peptide is a chelating peptide provided in Table 21

TABLE 21
Chelating Peptides

| Abbreviation | | SEQ |
|---|---|---|
| (if applicable | Amino Acid Sequence | ID NO |
| HIS5 | His-His-His-His-His | 531. |
| HIS6 | His-His-His-His-His-His | 532. |
| HIS7 | His-His-His-His-His-His-His | 533. |
| HIS8 | His-His-His-His-His-His-His- | 534. |
| | His | |
| HIS9 | His-His-His-His-His-His-His- | 535. |
| | His-His | |
| n/a | His-Trp-His-Met-Tyr | 536. |
| n/a | His-His-His-Met-Tyr | 537. |
| n/a | His-His-His-His-Tyr | 538. |
| n/a | His-Trp-His-Trp-His | 539. |
| n/a | His-Trp-His-His-His | 540. |
| n/a | His-His-His-His-Tyr-Met-His- | 541. |
| | His-His- | |
| n/a | His-His-His-His-His | 542. |
| n/a | His-Trp-His-His-His- | 543. |
| n/a | His-Gly-His-Gly-Gly-Gly-His- | 544. |
| | Gly-His | |
| n/a | His-Gly-His-Gly-Gly-Gly-Gly- | 545. |
| | Gly-Gly-His-Gly-His | |

### Elimination of N-Linked Glycosylation Sites

In some embodiments, it is possible that an amino acid sequence (particularly a CDR sequence) of the IL10Ra or IL10Rb sdAb may contain a glycosylation motif, particularly an N-linked glycosylation motif of the sequence Asn-X-Ser (N-X-S) or Asn-X-Thr (N-X-T), wherein X is any amino acid except for proline. In such instances, it is desirable to eliminate such N-linked glycosylation motifs by modifying the sequence of the N-linked glycosylation motif to prevent glycosylation. In some embodiments, the elimination of the Asn-X-Ser (N-X-S) N-linked glycosylation motif may be achieved by the incorporation of conservative amino acid substitution of the Asn (N) residue and/or Ser(S) residue of the Asn-X-Ser (N-X-S) N-linked glycosylation motif. In some embodiments, the elimination of the Asn-X-Thr (N-X-T) N-linked glycosylation motif may be achieved by the incorporation of conservative amino acid substitution of the Asn (N) residue and/or Thr (T) residue of the Asn-X-Thr (N-X-T) N-linked glycosylation motif. In some embodiments, elimination of the glycosylation site is not required when the IL10R binding molecule comprising the IL10Ra or IL10Rb sdAb is expressed in procaryotic host cells. Since procaryotic cells do not provide a mechanism for glycosylation of recombinant proteins, when employing a procaryotic expression system to produce a recombinant IL10R binding molecule comprising the IL10Ra or IL10Rb sdAb the modification of the sequence to eliminate the N-linked glycosylation sites may be obviated.

Association with Carrier Molecules to Increase Duration of Action

The IL10R binding molecule described herein can be modified to provide for an extended lifetime in vivo and/or extended duration of action in a subject. In some embodiments, the binding molecule can be conjugated to carrier molecules to provide desired pharmacological properties such as an extended half-life. In some embodiments, the binding molecule can be covalently linked to the Fc domain of IgG, albumin, or other molecules to extend its half-life, e.g., by pegylation, glycosylation, and the like as known in the art. In some embodiments, the IL10R binding molecule modified to provide an extended duration of action in a mammalian subject has a half-life in a mammalian of greater than 4 hours, alternatively greater than 5 hours, alternatively greater than 6 hours, alternatively greater than 7 hours, alternatively greater than 8 hours, alternatively greater than 9 hours, alternatively greater than 10 hours, alternatively greater than 12 hours, alternatively greater than 18 hours, alternatively greater than 24 hours, alternatively greater than 2 days, alternatively greater than 3 days, alternatively greater than 4 days, alternatively greater than 5 days, alternatively greater than 6 days, alternatively greater than 7 days, alternatively greater than 10 days, alternatively greater than 14 days, alternatively greater than 21 days, or alternatively greater than 30 days.

Modifications of the IL10R binding molecule to provide an extended duration of action in a mammalian subject include (but are not limited to);
conjugation of the IL10R binding molecule to one or more carrier molecules,
conjugation IL10R binding molecule to protein carriers molecules, optionally in the form of a fusion protein with additional polypeptide sequences (e.g, IL10R binding molecule-Fc fusions) and
conjugation to polymers, (e.g. water soluble polymers to provide a PEGylated IL10R binding molecule).

It should be noted that the more than one type of modification that provides for an extended duration of action in a mammalian subject may be employed with respect to a given IL10R binding molecule. For example, IL10R binding molecule of the present disclosure may comprise both amino acid substitutions that provide for an extended duration of action as well as conjugation to a carrier molecule such as a polyethylene glycol (PEG) molecule.

### Protein Carrier Molecules:

Examples of protein carrier molecules which may be covalently attached to the IL10R binding molecule to provide an extended duration of action in vivo include, but are not limited to albumins, antibodies and antibody fragments such and Fc domains of IgG molecules

### Fc Fusions:

In some embodiments, the IL10R binding molecule is conjugated to a functional domain of an Fc-fusion chimeric polypeptide molecule. Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates. The “Fc region” useful in the preparation of Fc fusions can be a naturally occurring or synthetic polypeptide that is homologous to an IgG C-terminal domain produced by digestion of IgG with papain. IgG Fc has a molecular weight of approximately 50 kDa. The binding molecule described herein can be conjugated to the entire Fc region, or a smaller portion that retains the ability to extend the circulating half-life of a chimeric polypeptide of which it is a part. In addition, full-length or fragmented Fc regions can be variants of the wild-type molecule. In a typical presentation, each monomer of the dimeric Fc can carry a heterologous polypeptide, the heterologous polypeptides being the same or different.

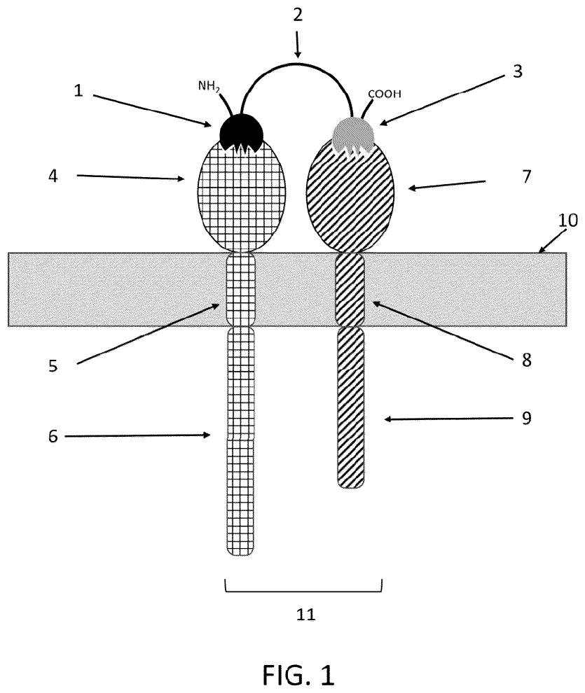

Illustrative examples of Fc formats useful for IL10R binding molecules of the present disclosure are provided schematically in FIGS. 1-4 of the attached drawings.

### Linkage of Binding Molecule to Fc Monomer

As indicated, the linkage of the IL10R binding molecule to the Fc subunit may incorporate a linker molecule as described below between the IL10R binding molecule and Fc subunit. In some embodiments, the IL10R binding molecule is expressed as a fusion protein with the Fc domain incorporating an amino acid sequence of a hinge region of an IgG antibody. The Fc domains engineered in accordance with the foregoing may be derived from IgG1, IgG2, IgG3 and IgG4 mammalian IgG species. In some embodiments, the Fc domains may be derived from human IgG1, IgG2, IgG3 and IgG4 IgG species. In some embodiments, the hinge region is the hinge region of an IgG1. In one particular embodiment, the IL10R binding molecule is linked to an Fc domain using an human IgG1 hinge domain.

### Knob-into-Hole Fc Format

In some embodiments, the dimeric Fc molecule may be engineered to possess a “knob-into-hole modification.”

### Albumin Carrier Molecules

In some embodiments, the IL10R binding molecule conjugated to an is albumin molecule (e.g., human serum albumin) which is known in the art to facilitate extended exposure in vivo. In one embodiment of the invention, the IL10R binding molecule is conjugated to albumin via chemical linkage or expressed as a fusion protein with an albumin molecule referred to herein as an IL10R binding molecule albumin fusion.” The term “albumin” as used in the context αβhIL2 mutein albumin fusions include albumins such as human serum albumin (HSA), cyno serum albumin, and bovine serum albumin (BSA). In some embodiments, the HSA the HSA comprises a C34S or K573P amino acid substitution relative to the wild-type HSA sequence According to the present disclosure, albumin can be conjugated to a IL10R binding molecule at the carboxyl terminus, the amino terminus, both the carboxyl and amino termini, and internally (see, e.g., U.S. Pat. Nos. 5,876,969 and 7,056,701). In the HAS IL10R binding molecule contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising a IL10R binding molecule fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. As an alternative to chemical linkage between the IL10R binding molecule and the albumin molecule the IL10R binding molecule-albumin complex may be provided as a fusion protein comprising an albumin polypeptide sequence and an IL10R binding molecule recombinantly expressed in a host cell as a single polypeptide chain, optionally comprising a linker molecule between the albumin and IL10R binding molecules\. Such fusion proteins may be readily prepared through recombinant technology to those of ordinary skill in the art. Nucleic acid sequences encoding such fusion proteins may be ordered from any of a variety of commercial sources. The nucleic acid sequence encoding the fusion protein is incorporated into an expression vector operably linked to one or more expression control elements, the vector introduced into a suitable host cell and the fusion protein solated from the host cell culture by techniques well known in the art.

### Polymeric Carriers

In some embodiments, extended in vivo duration of action of the IL10R binding molecule may be achieved by conjugation to one or more polymeric carrier molecules such as XTEN polymers or water soluble polymers.

### XTEN Conjugates

The IL10R binding molecule may further comprise an XTEN polymer. The XTEN polymer may be is conjugated (either chemically or as a fusion protein) the αβhIL2 mutein provides extended duration of akin to PEGylation and may be produced as a recombinant fusion protein in E. coli. XTEN polymers suitable for use in conjunction with the IL10R binding molecule of the present disclosure are provided in Podust, et al. (2016) “Extension of in vivo half-life of biologically active molecules by XTEN protein polymers”, J Controlled Release 240:52-66 and Haeckel et al. (2016) “XTEN as Biological Alternative to PEGylation Allows Complete Expression of a Protease-Activatable Killin-Based Cytostatic” PLOS ONE|DOI: 10.1371/journal.pone.0157193 Jun. 13, 2016. The XTEN polymer may fusion protein may incorporate a protease sensitive cleavage site between the XTEN polypeptide and the hIL2 mutein such as an MMP-2 cleavage site.

### Water Soluble Polymers

In some embodiments, the IL10R binding molecule can be conjugated to one or more water-soluble polymers. Examples of water soluble polymers useful in the practice of the present disclosure include polyethylene glycol (PEG), poly-propylene glycol (PPG), polysaccharides (polyvinylpyrrolidone, copolymers of ethylene glycol and propylene glycol, poly(oxyethylated polyol), polyolefinic alcohol,), polysaccharides), poly-alpha-hydroxy acid), polyvinyl alcohol (PVA), polyphosphazene, polyoxazolines (POZ), poly(N-acryloylmorpholine), or a combination thereof.

In some embodiments, IL10R binding molecule can be conjugated to one or more polyethylene glycol molecules or “PEGylated.” Although the method or site of PEG attachment to the binding molecule may vary, in certain embodiments the PEGylation does not alter, or only minimally alters, the activity of the binding molecule.

PEGs suitable for conjugation to a polypeptide sequence are generally soluble in water at room temperature, and have the general formula

R(O—CH₂—CH₂)ₙO—R,

where R is hydrogen or a protective group such as an alkyl or an alkanol group, and where n is an integer from 1 to 1000. When R is a protective group, it generally has from 1 to 8 carbons. The PEG can be linear or branched. Branched PEG derivatives, “star-PEGs” and multi-armed PEGs are contemplated by the present disclosure.

In some instances, the sequences of IL10R binding molecules of the present disclosure provided in Tables 9 and 10 possess an N-terminal glutamine (“1Q”) residue. N-terminal glutamine residues have been observed to spontaneously cyclyize to form pyroglutamate (pE) at or near physiological conditions. (See e.g., Liu, et al (2011) J. Biol. Chem. 286 (13): 11211-11217). In some embodiments, the formation of pyroglutamate complicates N-terminal PEG conjugation particularly when aldehyde chemistry is used for N-terminal PEGylation. Consequently, when PEGylating the IL10R binding molecules of the present disclosure, particularly when aldehyde chemistry is to be employed, the IL10R binding molecules possessing an amino acid at position 1 (e.g., 1Q) are substituted at position 1 with an alternative amino acid or are deleted at position 1 (e.g., des-1Q). In some embodiments, the IL10R binding molecules of the present disclosure comprise an amino acid substitution selected from the group Q1E and Q1D.

In some embodiments, selective PEGylation of the IL10R binding molecules\, for example, by the incorporation of non-natural amino acids having side chains to facilitate selective PEG conjugation, may be employed. Specific PEGylation sites can be chosen such that PEGylation of the binding molecule does not affect its binding to the target receptors.

In certain embodiments, the increase in half-life is greater than any decrease in biological activity. PEGs suitable for conjugation to a polypeptide sequence are generally soluble in water at room temperature, and have the general formula R(O-CH2-CH2)nO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

A molecular weight of the PEG used in the present disclosure is not restricted to any particular range. The PEG component of the binding molecule can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa, or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons.

Two widely used first generation activated monomethoxy PEGs (mPEGs) are succinimdyl carbonate PEG (SC-PEG; see, e.g., Zalipsky, et al. (1992) Biotehnol. Appl. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

Pegylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General PEGylation strategies known in the art can be applied herein.

The PEG can be bound to a binding molecule of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the PEGylation of the binding molecules is facilitated by the incorporation of non-natural amino acids bearing unique side chains to facilitate site specific PEGylation. The incorporation of non-natural amino acids into polypeptides to provide functional moieties to achieve site specific PEGylation of such polypeptides is known in the art. See e.g., Ptacin et al., PCT International Application No. PCT/US2018/045257 filed Aug. 3, 2018 and published Feb. 7, 2019 as International Publication Number WO 2019/028419A1.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present disclosure include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g., Sunbright® ME-200AL, NOF), a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10 kDA linear PEG molecules (e.g., Sunbright® GL2-200AL3, NOF), a 20 kDa 2-arm branched PEG-NHS ester the 20 kDA PEG-NHS ester comprising two 10 kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20 kDA linear PEG molecules (e.g., Sunbright® GL2-400AL3), a 40 kDa 2-arm branched PEG-NHS ester the 40 kDA PEG-NHS ester comprising two 20 kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

In some embodiments, a linker can used to join the IL10R binding molecule and the PEG molecule. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of flexible linkers are described in Section IV. Further, a multimer (e.g., 1, 2, 3, 4, 5, 6, 7, 8, 9, 10, 10-20, 20-30, or 30-50) of these linker sequences may be linked together to provide flexible linkers that may be used to conjugate two molecules. Alternative to a polypeptide linker, the linker can be a chemical linker, e.g., a PEG-aldehyde linker. In some embodiments, the binding molecule is acetylated at the N-terminus by enzymatic reaction with N-terminal acetyltransferase and, for example, acetyl COA. Alternatively, or in addition to N-terminal acetylation, the binding molecule can be acetylated at one or more lysine residues, e.g., by enzymatic reaction with a lysine acetyltransferase. See, for example Choudhary et al. (2009) Science 325 (5942): 834-840.

In some embodiments, the present invention provides a IL10R binding molecules of formula #1 that is PEGylated, wherein the PEG is conjuaged to the IL10R binding molecule and the PEG is a linear or branched PEG molecule having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

### Fatty Acid Carriers

In some embodiments an IL10R binding molecule having an extended duration of action in a mammalian subject and useful in the practice of the present disclosure is achieved by covalent attachment of the IL10R binding molecule to a fatty acid molecule as described in Resh (2016) Progress in Lipid Research 63:120-131. Examples of fatty acids that may be conjugated include myristate, palmitate and palmitoleic acid. Myristoylate is typically linked to an N-terminal glycine but lysines may also be myristoylated. Palmitoylation is typically achieved by enzymatic modification of free cysteine —SH groups such as DHHC proteins catalyze S-palmitoylation. Palmitoleylation of serine and threonine residues is typically achieved enzymatically using PORCN enzymes. In some embodiments, the IL10R binding molecule is acetylated at the N-terminus by enzymatic reaction with N-terminal acetyltransferase and, for example, acetyl COA. Alternatively, or in addition to N-terminal acetylation, the IL10R binding molecule is acetylated at one or more lysine residues, e.g., by enzymatic reaction with a lysine acetyltransferase. See, for example Choudhary et al. (2009) Science 325 (5942): 834L2 ortho840.

### Modifications to Provide Additional Functions

In some embodiments, embodiment, the IL10R binding molecule may comprise a functional domain of a chimeric polypeptide. IL10R binding molecule fusion proteins of the present disclosure may be readily produced by recombinant DNA methodology by techniques known in the art by constructing a recombinant vector comprising a nucleic acid sequence comprising a nucleic acid sequence encoding the IL10R binding molecule in frame with a nucleic acid sequence encoding the fusion partner either at the N-terminus or C-terminus of the IL10R binding molecules\, the sequence optionally further comprising a nucleic acid sequence in frame encoding a linker or spacer polypeptide.

### FLAG Tags

In other embodiments, the IL10R binding molecule can be modified to include an additional polypeptide sequence that functions as an antigenic tag, such as a FLAG sequence. FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the binding molecule further comprises a C-terminal c-myc epitope tag.

### Targeting Moieties:

In some embodiments, IL10R binding molecule is conjugated to molecule which provides (“targeting domain”) to facilitate selective binding to particular cell type or tissue expressing a cell surface molecule that specifically binds to such targeting domain, optionally incorporating a linker molecule of from 1-40 (alternatively 2-20, alternatively 5-20, alternatively 10-20) amino acids between IL10R binding molecule sequence and the sequence of the targeting domain of the fusion protein.

In other embodiments, a chimeric polypeptide including a IL10R binding molecule and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135.

In some embodiments, the targeting moiety is an antibody that specifically binds to at least one cell surface molecule associated with a tumor cell (i.e. at least one tumor antigen) wherein the cell surface molecule associated with a tumor cell is selected from the group consisting of GD2, BCMA, CD19, CD33, CD38, CD70, GD2, IL3R 2, CD19, mesothelin, Her2, EpCam, Muc1, ROR1, CD133, CEA, EGRFRVIII, PSCA, GPC3, Pan-ErbB and FAP.

### Recombinant Production

Alternatively, the IL10R binding molecules of the present disclosure are produced by recombinant DNA technology. In the typical practice of recombinant production of polypeptides, a nucleic acid sequence encoding the desired polypeptide is incorporated into an expression vector suitable for the host cell in which expression will be accomplish, the nucleic acid sequence being operably linked to one or more expression control sequences encoding by the vector and functional in the target host cell. The recombinant protein may be recovered through disruption of the host cell or from the cell medium if a secretion leader sequence (signal peptide) is incorporated into the polypeptide.

### Nucleic Acid Sequences Encoding the IL10R Binding Molecules

In some embodiments, the IL10R binding molecule is produced by recombinant methods using a nucleic acid sequence encoding the IL10R binding molecule (or fusion protein comprising the IL10R binding molecules). The nucleic acid sequence encoding the desired αβhIL10R binding molecule can be synthesized by chemical means using an oligonucleotide synthesizer.

The nucleic acid molecules are not limited to sequences that encode polypeptides; some or all of the non-coding sequences that lie upstream or downstream from a coding sequence (e.g., the coding sequence of IL-2) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The nucleic acid molecules encoding the IL10R binding molecule (and fusions thereof) may contain naturally occurring sequences or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

Nucleic acid sequences encoding the IL10R binding molecule may be obtained from various commercial sources that provide custom made nucleic acid sequences. Amino acid sequence variants of the IL10R binding molecules of the present disclosure are prepared by introducing appropriate nucleotide changes into the coding sequence based on the genetic code which is well known in the art. Such variants represent insertions, substitutions, and/or specified deletions of, residues as noted. Any combination of insertion, substitution, and/or specified deletion is made to arrive at the final construct, provided that the final construct possesses the desired biological activity as defined herein.

Methods for constructing a DNA sequence encoding a IL10R binding molecule and expressing those sequences in a suitably transformed host include, but are not limited to, using a PCR-assisted mutagenesis technique. Mutations that consist of deletions or additions of amino acid residues to a IL10R binding molecule can also be made with standard recombinant techniques. In the event of a deletion or addition, the nucleic acid molecule encoding a IL10R binding molecule is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

A IL10R binding molecule of the present disclosure may be produced recombinantly not only directly, but also as a fusion polypeptide with a heterologous polypeptide, e.g. a signal sequence or other polypeptide having a specific cleavage site at the N-terminus or C-terminus of the mature IL10R binding molecules\. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. The inclusion of a signal sequence depends on whether it is desired to secrete the IL10R binding molecule from the recombinant cells in which it is made. If the chosen cells are prokaryotic, it generally is preferred that the DNA sequence not encode a signal sequence. When the recombinant host cell is a yeast cell such as Saccharomyces cerevisiae, the alpha mating factor secretion signal sequence may be employed to achieve extracellular secretion of the IL10R binding molecule into the culture medium as described in Singh, U.S. Pat. No. 7,198,919 B1 issued Apr. 3, 2007.

In the event the IL10R binding molecule to be expressed is to be expressed as a chimera (e.g., a fusion protein comprising a IL10R binding molecule and a heterologous polypeptide sequence), the chimeric protein can be encoded by a hybrid nucleic acid molecule comprising a first sequence that encodes all or part of the IL10R binding molecule and a second sequence that encodes all or part of the heterologous polypeptide. For example, subject IL10R binding molecules described herein may be fused to a hexa-/octa-histidine tag (SEQ ID NOS 532 and 534, respectively) to facilitate purification of bacterially expressed protein, or to a hemagglutinin tag to facilitate purification of protein expressed in eukaryotic cells. By first and second, it should not be understood as limiting to the orientation of the elements of the fusion protein and a heterologous polypeptide can be linked at either the N-terminus and/or C-terminus of the IL10R binding molecules\. For example, the N-terminus may be linked to a targeting domain and the C-terminus linked to a hexa-histidine tag (SEQ ID NO: 532) purification handle.

The complete amino acid sequence of the polypeptide (or fusion/chimera) to be expressed can be used to construct a back-translated gene. A DNA oligomer containing a nucleotide sequence coding a IL10R binding molecule can be synthesized. For example, several small oligonucleotides coding for portions of the desired polypeptide can be synthesized and then ligated. The individual oligonucleotides typically contain 5′ or 3′ overhangs for complementary assembly.

### Codon Optimization:

In some embodiments, the nucleic acid sequence encoding the IL10R binding molecule may be “codon optimized” to facilitate expression in a particular host cell type. Techniques for codon optimization in a wide variety of expression systems, including mammalian, yeast and bacterial host cells, are well known in the and there are online tools to provide for a codon optimized sequences for expression in a variety of host cell types. See e.g. Hawash, et al., (2017) 9:46-53 and Mauro and Chappell in Recombinant Protein Expression in Mammalian Cells: Methods and Protocols, edited by David Hacker (Human Press New York). Additionally, there are a variety of web based on-line software packages that are freely available to assist in the preparation of codon optimized nucleic acid sequences.

### Expression Vectors:

Once assembled (by synthesis, site-directed mutagenesis or another method), the nucleic acid sequence encoding an a IL10R binding molecule will be inserted into an expression vector. A variety of expression vectors for uses in various host cells are available and are typically selected based on the host cell for expression. An expression vector typically includes, but is not limited to, one or more of the following: an origin of replication, one or more marker genes, an enhancer element, a promoter, and a transcription termination sequence. Vectors include viral vectors, plasmid vectors, integrating vectors, and the like. Plasmids are examples of non-viral vectors.

To facilitate efficient expression of the recombinant polypeptide, the nucleic acid sequence encoding the polypeptide sequence to be expressed is operably linked to transcriptional and translational regulatory control sequences that are functional in the chosen expression host.

### Selectable Marker:

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

### Regulatory Control Sequences:

Expression vectors for a IL10R binding molecules of the present disclosure contain a regulatory sequence that is recognized by the host organism and is operably linked to nucleic acid sequence encoding the IL10R binding molecules\. The terms “regulatory control sequence,” “regulatory sequence” or “expression control sequence” are used interchangeably herein to refer to promoters, enhancers, and other expression control elements (e.g., polyadenylation signals). See, for example, Goeddel (1990) in Gene Expression Technology: Methods in Enzymology 185 (Academic Press, San Diego CA USA Regulatory sequences include those that direct constitute expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. In selecting an expression control sequence, a variety of factors understood by one of skill in the art are to be considered. These include, for example, the relative strength of the sequence, its controllability, and its compatibility with the actual DNA sequence encoding the subject a IL10R binding molecules\, particularly as regards potential secondary structures.

### Promoters:

In some embodiments, the regulatory sequence is a promoter, which is selected based on, for example, the cell type in which expression is sought. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

Transcription from vectors in mammalian host cells may be controlled, for example, by promoters obtained from the genomes of viruses such as polyoma virus, fowlpox virus, adenovirus (such as human adenovirus serotype 5), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

### Enhancers:

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter. Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Additional examples of marker or reporter genes include beta-lactamase, chloramphenicol acetyltransferase (CAT), adenosine deaminase (ADA), dihydrofolate reductase (DHFR), hygromycin-B-phosphotransferase (HPH), thymidine kinase (TK), lacZ (encoding beta-galactosidase), and xanthine guanine phosphoribosyltransferase (XGPRT). Those of skill in the art can readily determine whether a given regulatory element or selectable marker is suitable for use in a particular experimental context.

Proper assembly of the expression vector can be confirmed by nucleotide sequencing, restriction mapping, and expression of a biologically active polypeptide in a suitable host.

### Host Cells:

The present disclosure further provides prokaryotic or eukaryotic cells that contain and express a nucleic acid molecule that encodes a IL10R binding molecules\. A cell of the present disclosure is a transfected cell, i.e., a cell into which a nucleic acid molecule, for example a nucleic acid molecule encoding a mutant IL-2 polypeptide, has been introduced by means of recombinant DNA techniques. The progeny of such a cell are also considered within the scope of the present disclosure.

Host cells are typically selected in accordance with their compatibility with the chosen expression vector, the toxicity of the product coded for by the DNA sequences of this invention, their secretion characteristics, their ability to fold the polypeptides correctly, their fermentation or culture requirements, and the ease of purification of the products coded for by the DNA sequences. Suitable host cells for cloning or expressing the DNA in the vectors herein are the prokaryote, yeast, or higher eukaryote cells.

In some embodiments the recombinant IL10R binding molecule can also be made in eukaryotes, such as yeast or human cells. Suitable eukaryotic host cells include insect cells (examples of Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerenvisiae include pYepSecl (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)).

Examples of useful mammalian host cell lines are mouse L cells (L-M [TK-], ATCC #CRL-2648), monkey kidney CV1 line transformed by SV40 (COS-7, ATCC CRL 1651); human embryonic kidney line (HEK293 or HEK293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2). In mammalian cells, the expression vector's control functions are often provided by viral regulatory elements. For example, commonly used promoters are derived from polyoma, Adenovirus 2, cytomegalovirus, and Simian Virus 40.

The IL10R binding molecule may be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

In some embodiments, a IL10R binding molecule obtained will be glycosylated or unglycosylated depending on the host organism used to produce the mutein. If bacteria are chosen as the host then the a IL10R binding molecule produced will be unglycosylated. Eukaryotic cells, on the other hand, will typically result in glycosylation of the IL10R binding molecules\.

In some embodiments, it is possible that an amino acid sequence (particularly a CDR sequence) of an sdAb to be incorporated into a IL10R binding molecule may contain a glycosylation motif, particularly an N-linked glycosylation motif of the sequence Asn-X-Ser (N-X-S) or Asn-X-Thr (N-X-T), wherein X is any amino acid except for proline. In such instances, it is desirable to eliminate such N-linked glycosylation motifs by modifying the sequence of the N-linked glycosylation motif to prevent glycosylation. In some embodiments, the N-linked glycosylation motif is disrupted by the incorporation of conservative amino acid substitution of the Asn (N) residue of the N-linked glycosylation motif.

For other additional expression systems for both prokaryotic and eukaryotic cells, see Chapters 16 and 17 of Sambrook et al. (1989) Molecular Cloning: A Laboratory Manual (2nd ed., Cold Spring Harbor Laboratory Press, Plainview, N.Y.). See, Goeddel (1990) in Gene Expression Technology: Methods in Enzymology 185 (Academic Press, San Diego, Calif.).

### Transfection:

The expression constructs of the can be introduced into host cells to thereby produce a IL10R binding molecule disclosed herein. The expression vector comprising a nucleic acic sequence encoding IL10R binding molecule is introduced into the prokaryotic or eukaryotic host cells via conventional transformation or transfection techniques. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals. To facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, and magnetic fields (electroporation).

### Cell Culture:

Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

### Recovery of Recombinant Proteins:

Recombinantly produced IL10R binding molecule polypeptides can be recovered from the culture medium as a secreted polypeptide if a secretion leader sequence is employed. Alternatively, the IL10R binding molecule polypeptides can also be recovered from host cell lysates. A protease inhibitor, such as phenyl methyl sulfonyl fluoride (PMSF) may be employed during the recovery phase from cell lysates to inhibit proteolytic degradation during purification, and antibiotics may be included to prevent the growth of adventitious contaminants.

Various purification steps are known in the art and find use, e.g. affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural specific binding of one molecular species to separate and purify a second species from a mixture. Antibodies are commonly used in affinity chromatography. Size selection steps may also be used, e.g. gel filtration chromatography (also known as size-exclusion chromatography or molecular sieve chromatography) is used to separate proteins according to their size. In gel filtration, a protein solution is passed through a column that is packed with semipermeable porous resin. The semipermeable resin has a range of pore sizes that determines the size of proteins that can be separated with the column.

A recombinantly IL10R binding molecule by the transformed host can be purified according to any suitable method. Recombinant IL10R binding molecules can be isolated from inclusion bodies generated in E. coli, or from conditioned medium from either mammalian or yeast cultures producing a given mutein using cation exchange, gel filtration, and or reverse phase liquid chromatography. The substantially purified forms of the recombinant a IL10R binding molecule can be purified from the expression system using routine biochemical procedures, and can be used, e.g., as therapeutic agents, as described herein.

In some embodiments, where the IL10R binding molecule is expressed with a purification tag as discussed above, this purification handle may be used for isolation of the IL10R binding molecule from the cell lysate or cell medium. Where the purification tag is a chelating peptide, methods for the isolation of such molecules using immobilized metal affinity chromatography are well known in the art. See, e.g., Smith, et al. U.S. Pat. No. 4,569,794.

The biological activity of the IL10R binding molecules recovered can be assayed for activating by any suitable method known in the art and may be evaluated as substantially purified forms or as part of the cell lysate or cell medium when secretion leader sequences are employed for expression.

### Pharmaceutical Formulations

In some embodiments, the subject IL10R binding molecule (and/or nucleic acids encoding the IL10R binding molecule or recombinant cells incorporating a nucleic acid sequence and modified to express the IL10R binding molecules\) can be incorporated into compositions, including pharmaceutical compositions. Such compositions typically include the polypeptide or nucleic acid molecule and a pharmaceutically acceptable carrier. A pharmaceutical composition is formulated to be compatible with its intended route of administration and is compatible with the therapeutic use for which the IL10R binding molecule is to be administered to the subject in need of treatment or prophyaxis.

### Carriers:

Carriers include a sterile diluent such as water for injection, saline solution, fixed oils, polyethylene glycols, glycerine, propylene glycol or other synthetic solvents. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. For intravenous administration, suitable carriers include physiological saline, bacteriostatic water, Cremophor EL™ (BASF, Parsippany, N.J.) or phosphate buffered saline (PBS).

### Buffers:

The term buffers includes buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5).

### Dispersions:

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### Preservatives:

The pharmaceutical formulations for parenteral administration to a subject should be sterile and should be fluid to facilitate easy syringability. It should be stable under the conditions of manufacture and storage and are preserved against the contamination. Prevention of the action of microorganisms can be achieved by various antibacterial and antifungal agents, for example, agents such as benzyl alcohol or methyl parabens; antioxidants such as ascorbic acid or sodium bisulfite; chelating agents such as ethylenediaminetetraacetic acid, parabens, chlorobutanol, phenol, ascorbic acid, thimerosal, and the like. Sterile solutions can be prepared by incorporating the active compound in the required amount in an appropriate solvent with one or a combination of ingredients enumerated above, as required, followed by filtered sterilization.

### Tonicity Agents:

In many cases, it will be preferable to include isotonic agents, for example, sugars, polyalcohols such as mannitol, sorbitol, sodium chloride in the composition.

### Routes of Administration:

In some embodiments of the therapeutic methods of the present disclosure involve the administration of a pharmaceutical formulation comprising a IL10R binding molecule (and/or nucleic acids encoding the IL10R binding molecule or recombinantly modified host cells expressing the IL10R binding molecules) to a subject in need of treatment. The pharmaceutical formulation comprising a IL10R binding molecules of the present disclosure may be administered to a subject in need of treatment or prophyaxis by a variety of routes of administration, including parenteral administration, oral, topical, or inhalation routes.

### Parenteral Administration:

In some embodiments, the methods of the present disclosure involve the parenteral administration of a pharmaceutical formulation comprising a IL10R binding molecule (and/or nucleic acids encoding the IL10R binding molecule or recombinantly modified host cells expressing the IL10R binding molecules\) to a subject in need of treatment. Examples of parenteral routes of administration include, for example, intravenous, intradermal, subcutaneous, transdermal (topical), transmucosal, and rectal administration. Parenteral formulations comprise solutions or suspensions used for parenteral application can include vehicles the carriers and buffers. Pharmaceutical formulations for parenteral administration include sterile aqueous solutions (where water soluble) or dispersions and sterile powders for the extemporaneous preparation of sterile injectable solutions or dispersion. The parenteral preparation can be enclosed in ampoules, disposable syringes or multiple dose vials made of glass or plastic. In one embodiment, the formulation is provided in a prefilled syringe for

### Oral Administration:

In some embodiments, the methods of the present disclosure involve the oral administration of a pharmaceutical formulation comprising a IL10R binding molecule (and/or nucleic acids encoding the IL10R binding molecule or recombinantly modified host cells expressing the IL10R binding molecules\) to a subject in need of treatment. Oral compositions, if used, generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

### Inhalation Formulations:

In some embodiments, the methods of the present disclosure involve the inhaled administration of a pharmaceutical formulation comprising a IL10R binding molecule (and/or nucleic acids encoding the IL10R binding molecule or recombinantly modified host cells expressing the IL10R binding molecules\) to a subject in need of treatment. In the event of administration by inhalation, subject IL10R binding molecules, or the nucleic acids encoding them, are delivered in the form of an aerosol spray from pressured container or dispenser which contains a suitable propellant, e.g., a gas such as carbon dioxide, or a nebulizer. Such methods include those described in U.S. Pat. No. 6,468,798.

### Mucosal and Transdermal Formulations:

In some embodiments, the methods of the present disclosure involve the mucosal or transdermal administration of a pharmaceutical formulation comprising a IL10R binding molecule (and/or nucleic acids encoding the IL10R binding molecule or recombinantly modified host cells expressing the IL10R binding molecules\) to a subject in need of treatment. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories suppositories (e.g., with conventional suppository bases such as cocoa butter and other glycerides) or retention enemas for rectal delivery. For transdermal administration, the active compounds are formulated into ointments, salves, gels, or creams as generally known in the art and may incorporate permeation enhancers such as ethanol or lanolin.

### Extended Release and Depot Formulations:

In some embodiments of the method of the present disclosure, the IL10R binding molecule is administered to a subject in need of treatment in a formulation to provide extended release of the IL10R binding molecule agent. Examples of extended release formulations of the injectable compositions can be brought about by including in the composition an agent which delays absorption, for example, aluminum monostearate and gelatin. In one embodiment, the subject IL10R binding molecules or nucleic acids are prepared with carriers that will protect the IL10R binding molecules against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

### Administration of Nucleic Acids Encoding the IL10R Binding Molecules\:

In some embodiments of the method of the present disclosure, delivery of the IL10R binding molecule to a subject in need of treatment is achieved by the administration of a nucleic acid encoding the IL10R binding molecules\. Methods for the adminstration nucleic acid encoding the IL10R binding molecule to a subject is achieved by transfection or infection using methods known in the art, including but not limited to the methods described in McCaffrey et al. (Nature (2002) 418:6893), Xia et al. (Nature Biotechnol. (2002) 20:1006-1010), or Putnam (Am. J. Health Syst. Pharm. (1996) 53:151-160 erratum at Am. J. Health Syst. Pharm. (1996) 53:325). In some embodiments, the IL10R binding molecule is administered to a subject by the administration of a pharmaceutically acceptable formulation of recombinant expression vector comprising a nucleic acid sequence encoding the IL10R binding molecule operably linked to one or more expression control sequences operable in a mammalian subject. In some embodiments, the expression control sequence may be selected that is operable in a limited range of cell types (or single cell type) to facilitate the selective expression of the IL10R binding molecule in a particular target cell type. In one embodiment, the recombinant expression vector is a viral vector. In some embodiments, the recombinant vector is a recombinant viral vector. In some embodiments the recombinant viral vector is a recombinant adenoassociated virus (rAAV) or recombinant adenovirus (rAd), in particular a replication deficient adenovirus derived from human adenovirus serotypes 3 and/or 5. In some embodiments, the replication deficient adenovirus has one or more modifications to the E1 region which interfere with the ability of the virus to initiate the cell cycle and/or apoptotic pathways in a human cell. The replication deficient adenoviral vector may optionally comprise deletions in the E3 domain. In some embodiments the adenovirus is a replication competent adenovirus. In some embodiments the adenovirus is a replication competent recombinant virus engineered to selectively replicate in the target cell type.

In some embodiments, particularly for administration of IL10R binding molecules to the subject, particular for treatment of diseases of the intestinal tract or bacterial infections in a subject, the nucleic acid encoding the IL10R binding molecule may be delivered to the subject by the administration of a recombinantly modified bacteriophage vector encoding the IL10R binding molecules\. As used herein, the terms ‘procaryotic virus,” “bacteriophage” and “phage” are used interchangeably hereinto describe any of a variety of bacterial viruses that infect and replicate within a bacterium. Bacteriophage selectively infect procaryotic cells, restricting the expression of the IL10R binding molecule to procaryotic cells in the subject while avoiding expression in mammalian cells. A wide variety of bacteriophages capable of selection a broad range of bacterial cells have been identified and characterized extensively in the scientific literature. In some embodiments, the phage is modified to remove adjacent motifs (PAM). Elimination of the of Cas9 sequences from the phage genome reduces ability of the Cas9 endonuclease of the target procaryotic cell to neutralize the invading phage encoding the IL10R binding molecules.

### Administration of Recombinantly Modified Cells Expressing the IL10R Binding Molecule

In some embodiments of the method of the present disclosure, delivery of the IL10R binding molecule to a subject in need of treatment is achieved by the administration of recombinant host cells modified to express the IL10R binding molecule may be administered in the therapeutic and prophylactic applications described herein. In some embodiments, the recombinant host cells are mammalian cells, e.g., human cells.

In some embodiments, the nucleic acid sequence encoding the IL10R binding molecule (or vectors comprising same) may be maintained extrachromosomally in the recombinantly modified host cell for administration. In other embodiments, the nucleic acid sequence encoding the IL10R binding molecule may be incorporated into the genome of the host cell to be administered using at least one endonuclease to facilitate incorporate insertion of a nucleic acid sequence into the genomic sequence of the cell. As used herein, the term “endonuclease” is used to refer to a wild-type or variant enzyme capable of catalyzing the cleavage of bonds between nucleic acids within a DNA or RNA molecule, preferably a DNA molecule. Endonucleases are referred to as “rare-cutting” endonucleases when such endonucleases have a polynucleotide recognition site greater than about 12 base pairs (bp) in length, more preferably of 14-55 bp. Rare-cutting endonucleases can be used for inactivating genes at a locus or to integrate transgenes by homologous recombination (HR) i.e. by inducing DNA double-strand breaks (DSBs) at a locus and insertion of exogenous DNA at this locus by gene repair mechanism. Examples of rare-cutting endonucleases include homing endonucleases (Grizot, et al (2009) Nucleic Acids Research 37 (16): 5405-5419), chimeric Zinc-Finger nucleases (ZFN) resulting from the fusion of engineered zinc-finger domains (Porteus M and Carroll D., Gene targeting using zinc finger nucleases (2005) Nature Biotechnology 23 (3): 967-973, a TALEN-nuclease, a Cas9 endonuclease from CRISPR system as or a modified restriction endonuclease to extended sequence specificity (Eisenschmidt, et al. 2005; 33 (22): 7039-7047).

In some embodiments, particularly for administration of IL10R binding molecules to the intestinal tract, the IL10R binding molecule may be delivered to the subject by a recombinantly modified procaryotic cell (e.g., Lactobacillus lacti). The use of engineered procaryotic cells for the delivery of recombinant proteins to the intestinal tract are known in the art. See, e.g. Lin, et al. (2017) Microb Cell Fact 16:148. In some embodiments, the engineered bacterial cell expressing the IL10R binding molecule may be administered orally, typically in aqueous suspension, or rectally (e.g. enema).

### Methods of Use

The present disclosure further provides methods of treating a subject suffering from a disease disorder or condition by the administration of a therapeutically effective amount of an IL10R binding molecule (or nucleic acid encoding an IL10R binding molecule including recombinant viruses encoding the IL10R binding molecules\) of the present disclosure.

### Inflammatory and Autoimmune Disorders

Disorders amenable to treatment with an IL10R binding molecule (including pharmaceutically acceptable formulations comprising an IL10R binding molecules and/or the nucleic acid molecules that encode them including recombinant viruses encoding such an IL10R binding molecules) of the present disclosure include inflammatory or autoimmune diseases including but not limited to, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes including Type 1 or type 2 diabetes, inflammation, autoimmune disease, atopic diseases, paraneoplastic autoimmune diseases, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome (Seronegativity Enthesopathy Arthropathy Syndrome), juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, SEA Syndrome (Seronegativity, Enthesopathy, Arthropathy Syndrome).

Other examples of proliferative and/or differentiative disorders amenable to treatment with IL10R binding molecules (including pharmaceutically acceptable formulations comprising IL10R binding molecules and/or the nucleic acid molecules that encode them including recombinant viruses encoding such IL10R binding molecules) of the present disclosure include, but are not limited to, skin disorders. The skin disorder may involve the aberrant activity of a cell or a group of cells or layers in the dermal, epidermal, or hypodermal layer, or an abnormality in the dermal-epidermal junction. For example, the skin disorder may involve aberrant activity of keratinocytes (e.g., hyperproliferative basal and immediately suprabasal keratinocytes), melanocytes, Langerhans cells, Merkel cells, immune cell, and other cells found in one or more of the epidermal layers, e.g., the stratum basale (stratum germinativum), stratum spinosum, stratum granulosum, stratum lucidum or stratum corneum. In other embodiments, the disorder may involve aberrant activity of a dermal cell, for example, a dermal endothelial, fibroblast, immune cell (e.g., mast cell or macrophage) found in a dermal layer, for example, the papillary layer or the reticular layer.

Examples of inflammatory or autoimmune skin disorders include psoriasis, psoriatic arthritis, dermatitis (eczema), for example, exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitises such as seborrheic dermatitis and solar dermatitis; keratoses such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis. The skin disorder can be dermatitis, e.g., atopic dermatitis or allergic dermatitis, or psoriasis.

The compositions of the present disclosure (including pharmaceutically acceptable formulations comprising IL10R binding molecules and/or the nucleic acid molecules that encode them including recombinant viruses encoding such IL10R binding molecules) can also be administered to a patient who is suffering from (or may suffer from) psoriasis or psoriatic disorders. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis. Examples of psoriatic disorders include chronic stationary psoriasis, plaque psoriasis, moderate to severe plaque psoriasis, psoriasis vulgaris, eruptive psoriasis, psoriatic erythroderma, generalized pustular psoriasis, annular pustular psoriasis, or localized pustular psoriasis.

### Treatment of Neoplastic Disease

The present disclosure provides methods of use of IL10R binding molecules in the treatment of a subject suffering from a neoplastic disease disorder or condition by the administration of a therapeutically effective amount of an IL10R binding molecule (or nucleic acid encoding an IL10R binding molecule including recombinant vectors encoding IL10R binding molecules) as described herein.

The compositions and methods of the present disclosure are useful in the treatment of subject suffering from a neoplastic disease characterized by the presence neoplasms, including benign and malignant neoplasms, and neoplastic disease.

Examples of benign neoplasms amenable to treatment using the compositions and methods of the present disclosure include but are not limited to adenomas, fibromas, hemangiomas, and lipomas. Examples of pre-malignant neoplasms amenable to treatment using the compositions and methods of the present disclosure include but are not limited to hyperplasia, atypia, metaplasia, and dysplasia. Examples of malignant neoplasms amenable to treatment using the compositions and methods of the present disclosure include but are not limited to carcinomas (cancers arising from epithelial tissues such as the skin or tissues that line internal organs), leukemias, lymphomas, and sarcomas typically derived from bone fat, muscle, blood vessels or connective tissues). Also included in the term neoplasms are viral induced neoplasms such as warts and EBV induced disease (i.e., infectious mononucleosis), scar formation, hyperproliferative vascular disease including intimal smooth muscle cell hyperplasia, restenosis, and vascular occlusion and the like.

The term “neoplastic disease” includes cancers characterized by solid tumors and non-solid tumors including but not limited to breast cancers; sarcomas (including but not limited to osteosarcomas and angiosarcomas and fibrosarcomas), leukemias, lymphomas, genitourinary cancers (including but not limited to ovarian, urethral, bladder, and prostate cancers); gastrointestinal cancers (including but not limited to colon esophageal and stomach cancers); lung cancers; myelomas; pancreatic cancers; liver cancers; kidney cancers; endocrine cancers; skin cancers; and brain or central and peripheral nervous (CNS) system tumors, malignant or benign, including gliomas and neuroblastomas, astrocytomas, myelodysplastic disorders; cervical carcinoma-in-situ; intestinal polyposes; oral leukoplakias; histiocytoses, hyperprofroliferative scars including keloid scars, hemangiomas; hyperproliferative arterial stenosis, psoriasis, inflammatory arthritis; hyperkeratoses and papulosquamous eruptions including arthritis.

The term neoplastic disease includes carcinomas. The term “carcinoma” refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The term neoplastic disease includes adenocarcinomas. An “adenocarcinoma” refers to a carcinoma derived from glandular tissue or in which the tumor cells form recognizable glandular structures.

As used herein, the term “hematopoietic neoplastic disorders” refers to neoplastic diseases involving hyperplastic/neoplastic cells of hematopoietic origin, e.g., arising from myeloid, lymphoid or erythroid lineages, or precursor cells thereof.

Myeloid neoplasms include, but are not limited to, myeloproliferative neoplasms, myeloid and lymphoid disorders with eosinophilia, myeloproliferative/myelodysplastic neoplasms, myelodysplastic syndromes, acute myeloid leukemia and related precursor neoplasms, and acute leukemia of ambiguous lineage. Exemplary myeloid disorders amenable to treatment in accordance with the present disclosure include, but are not limited to, acute promyeloid leukemia (APML), acute myelogenous leukemia (AML) and chronic myelogenous leukemia (CML).

Lymphoid neoplasms include, but are not limited to, precursor lymphoid neoplasms, mature B-cell neoplasms, mature T-cell neoplasms, Hodgkin's Lymphoma, and immunodeficiency-associated lymphoproliferative disorders. Exemplary lymphic disorders amenable to treatment in accordance with the present disclosure include, but are not limited to, acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM).

In some instances, the hematopoietic neoplastic disorder arises from poorly differentiated acute leukemias (e.g., erythroblastic leukemia and acute megakaryoblastic leukemia). As used herein, the term “hematopoietic neoplastic disorders” refers malignant lymphomas including, but are not limited to, non-Hodgkins lymphoma and variants thereof, peripheral T cell lymphomas, adult T-cell leukemia/lymphoma (ATL), cutaneous T cell lymphoma (CTCL), large granular lymphocytic leukemia (LGF), Hodgkin's disease and Reed-Stemberg disease.

The determination of whether a subject is “suffering from a neoplastic disease” refers to a determination made by a physician with respect to a subject based on the available information accepted in the field for the identification of a disease, disorder or condition including but not limited to X-ray, CT-scans, conventional laboratory diagnostic tests (e.g. blood count, etc.), genomic data, protein expression data, immunohistochemistry, that the subject requires or will benefit from treatment.

The adaptive immune system recognizes the display of certain cell surface proteins in response to tumor mutations facilitating the recognition and elimination of neoplastic cells. Tumors that possess a higher tumor mutation burden (TMB) are more likely to exhibit such “tumor antigens.” Indeed, clinical experience shows that tumors comprised of neoplastic cells exhibiting a high tumor mutation burden are more likely to respond to immune therapies, including immune checkpoint blockade (Rizvi, et al. (2015) Science 348 (6230): 124-128; Marabelle, et al. (2020) Lancet Oncol 21 (10): 1353-1365). Tumor mutation burden is useful as a biomarker to identify tumors with an increased sensitivity to immune therapies such as those provided in the present disclosure.

In some embodiments, the compositions and methods of the present disclosure are useful in the treatment of neoplastic disease associated with the formation of solid tumors exhibiting an intermediate or high tumor mutational burden (TMB). In some embodiments, the compositions and compositions and methods of the present disclosure are useful in the treatment of immune sensitive solid tumors exhibiting an intermediate or high tumor mutational burden (TMB). Examples of neoplastic diseases associated with the formation of solid tumors having an intermediate or high tumor mutational burden amenable to treatment with the compositions and methods of the present disclosure include, but are not limited to, non-small cell lung cancer and renal cell cancer. In one embodiment, the compositions and methods are useful in the treatment of non-small cell lung cancer (NSCLC) exhibiting an intermediate or high TMB. NSCLC cells typically harbor a significant number of mutations and are therefore more sensitive to immune therapies. The current standard of care for NSCLC is stratified by the cancer initiating mechanisms and generally follows the recommendations of NCCN or ASCO. A large proportion of NSCLC has increased TMB and is therefore initially more sensitive to immune therapies. However, most tumors eventually relapse on immune checkpoint inhibition. Patients with relapsed tumors typically show reduced T cell infiltration in the tumor, systemic T cell exhaustion and a suppressed immune response compared to the lesions prior to immune checkpoint inhibition. Therefore, improved immune therapies are required, re-activating and expanding the exhausted, rare tumor infiltrating T cells.

Combination of IL10R Binding Molecules with Supplemental Therapeutic Agents:

The present disclosure provides for the use of the IL10R binding molecules of the present disclosure in combination with one or more additional active agents (“supplemental agents”). Such further combinations are referred to interchangeably as “supplemental combinations” or “supplemental combination therapy” and those therapeutic agents that are used in combination with IL10R binding molecules of the present disclosure are referred to as “supplemental agents.” As used herein, the term “supplemental agents” includes agents that can be administered or introduced separately, for example, formulated separately for separate administration (e.g., as may be provided in a kit) and/or therapies that can be administered or introduced in combination with the hIL10R binding molecules.

As used herein, the term “in combination with” when used in reference to the administration of multiple agents to a subject refers to the administration of a first agent at least one additional (i.e. second, third, fourth, fifth, etc.) agent to a subject. For purposes of the present invention, one agent (e.g. hIL10R binding molecule) is considered to be administered in combination with a second agent (e.g. a modulator of an immune checkpoint pathway) if the biological effect resulting from the administration of the first agent persists in the subject at the time of administration of the second agent such that the therapeutic effects of the first agent and second agent overlap. For example, the PD1 immune checkpoint inhibitors (e.g. nivolumab or pembrolizumab) are typically administered by IV infusion every two weeks or every three weeks while the hIL10R binding molecules of the present disclosure are typically administered more frequently, e.g. daily, BID, or weekly. However, the administration of the first agent (e.g. pembrolizumab) provides a therapeutic effect over an extended time and the administration of the second agent (e.g. an hIL10R binding molecule) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject. In certain embodiments, the hIL10R binding molecule and the supplemental agent(s) are administered or applied sequentially, e.g., where one agent is administered prior to one or more other agents. In other embodiments, the hIL10R binding molecule and the supplemental agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

### Establishing Optimum Combinatorial Therapies:

Further embodiments comprise a method or model for determining the optimum amount of an agent(s) in a combination. An optimum amount can be, for example, an amount that achieves an optimal effect in a subject or subject population, or an amount that achieves a therapeutic effect while minimizing or eliminating the adverse effects associated with one or more of the agents. In some embodiments, the methods involving the combination of an hIL10R binding molecule and a supplemental agent which is known to be, or has been determined to be, effective in treating or preventing a disease, disorder or condition described herein (e.g., a cancerous condition) in a subject (e.g., a human) or a subject population, and an amount of one agent is titrated while the amount of the other agent(s) is held constant. By manipulating the amounts of the agent(s) in this manner, a clinician is able to determine the ratio of agents most effective for, for example, treating a particular disease, disorder or condition, or eliminating the adverse effects or reducing the adverse effects such that are acceptable under the circumstances.

### Supplemental Agents Useful in the Treatment of Inflammatory or Autoimmune Disorders

In some embodiments, the method further comprises administering of the IL10R binding molecule of the present disclosure in combination with one or more supplemental agents selected from the group consisting of a corticosteroid, a Janus kinase inhibitor, a calcineurin inhibitor, a mTor inhibitor, an IMDH inhibitor, a biologic, a vaccine, and a therapeutic antibody. In certain embodiments, the therapeutic antibody is an antibody that binds a protein selected from the group consisting of BLyS, CD11a, CD20, CD25, CD3, CD52, IgEIL12/IL23, IL17a, IL1ß, IL4Ra, IL5, IL6R, integrin-α4β7, RANKL, TNFα, VEGF-A, and VLA-4.

In some embodiments, the supplemental agent is one or more agents selected from the group consisting of corticosteroids (including but not limited to prednisone, budesonide, prednilisone), Janus kinase inhibitors (including but not limited to tofacitinib (Xeljanz®), calcineurin inhibitors (including but not limited to cyclosporine and tacrolimus), mTor inhibitors (including but not limited to sirolimus and everolimus), IMDH inhibitors (including but not limited to azathioprine, leflunomide and mycophenolate), biologics such as abatcept (Orencia®) or etanercept (Enbrel®), and therapeutic antibodies.

Examples of therapeutic antibodies that may be administered as supplemental agents in combination with the IL10R binding molecules of the present disclosure in the treatment of autoimmune disease include but are not limited to anti-CD25 antibodies (e.g. daclizumab and basiliximab), anti-VLA-4 antibodies (e.g. natalizumab), anti-CD52 antibodies (e.g. alemtuzumab), anti-CD20 antibodies (e.g. rituximab, ocrelizumab), anti-TNF antibodies (e.g. infliximab, and adalimumab), anti-IL6R antibodies (e.g. tocilizumab), anti-TNFα antibodies (e.g. adalimumab (Humira®), golimumab, and infliximab), anti-integrin-α4β7 antibodies (e.g. vedolizumab), anti-IL17a antibodies (e.g. brodalumab or secukinumab), anti-IL4Rα antibodies (e.g. dupilumab), anti-RANKL antibodies, IL6R antibodies, anti-IL1ß antibodies (e.g. canakinumab), anti-CD11a antibodies (e.g. efalizumab), anti-CD3 antibodies (e.g. muramonab), anti-IL5 antibodies (e.g. mepolizumab, reslizumab), anti-BLyS antibodies (e.g. belimumab); and anti-IL12/IL23 antibodies (e.g ustekinumab). Many therapeutic antibodies have been approved for clinical use against autoimmune disease. Examples of antibodies approved by the United States Food and Drug Administration (FDA) for use in the treatment of autoimmune diseases in a subject suffering therefrom that may be administered as supplemental agents in combination with the IL10R binding molecules of the present disclosure (and optionally additional supplemental agents) for the treatment of the indicated autoimmune disease include but are not limited toatezolizumab, olaratumab, ixekizumab, trastuzumab, infliximab, rituximab, edrecolomab, daratumumab, elotuzumab, necitumumab, dinutuximab, nivolumab, blinatumomab, pembrolizumab, pertuzumab, brentuximab vedotin, ipilimumab, ofatumumab, certolizumab pegol, catumaxomab, panitumumab, bevacizumab, ramucirumab, siltuximab, enfortumab vedotin, polatuzumab vedotin, [fam]-trastuzumab deruxtecan, cemiplimab, moxetumomab pasudotox, mogamuizumab, tildrakizumab, ibalizumab, durvalumab, inotuzumab, ozogamicin, avelumab, obinutuzumab, ado-trastuzumab emtansine, cetuximab, tositumomab-1131, ibritumomab tiuxetan, gemtuzumab, and ozogamicin.

The foregoing antibodies useful as supplemental agents in the practice of the methods of the present disclosure may be administered alone or in the form of any antibody drug conjugate (ADC) comprising the antibody, linker, and one or more drugs (e.g. 1, 2, 3, 4, 5, 6, 7, or 8 drugs) or in modified form (e.g. PEGylated).

### Supplemental Agents Useful in the Treatment of Neoplastic Disease:

In some embodiments, the supplemental agent is a chemotherapeutic agent. In some embodiments the supplemental agent is a “cocktail” of multiple chemotherapeutic agents. IN some embodiments the chemotherapeutic agent or cocktail is administered in combination with one or more physical methods (e.g. radiation therapy). The term “chemotherapeutic agents” includes but is not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins such as bleomycin A2, cactinomycin, calicheamicin, carabicin, caminomycin, carzinophilin, chromomycins, dactinomycin, daunorubicin and derivaties such as demethoxy-daunomycin, 11-deoxydaunorubicin, 13-deoxydaunorubicin, detorubicin, 6-diazo-5-oxo-L-norleucine, doxorubicin, epirubicin, esorubicin, idarubicin, marcellomycin, mitomycins such as mitomycin C, N-methyl mitomycin C; mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate, dideazatetrahydrofolic acid, and folinic acid; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab-paclitaxel and doxetaxel; chlorambucil; gemcitabine; 6-thioguanine; mercaptopurine; methotrexate; platinum and platinum coordination complexes such as cisplatin, oxaplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; taxanes such as paclitaxel, docetaxel, cabazitaxel; carminomycin, adriamycins such as 4′-epiadriamycin, 4-adriamycin-14-benzoate, adriamycin-14-octanoate, adriamycin-14-naphthaleneacetate; cholchicine and pharmaceutically acceptable salts, acids or derivatives of any of the above.

The term “chemotherapeutic agents” also includes anti-hormonal agents that act to regulate or inhibit hormone action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above.

In some embodiments, a supplemental agent isone or more chemical or biological agents identified in the art as useful in the treatment of neoplastic disease, including, but not limited to, a cytokines or cytokine antagonists such as IL-12, INFα, or anti-epidermal growth factor receptor, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, non-steroidal anti-inflammatory drugs (NSAIDs), cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foreoing as practied in known chemotherapeutic treatment regimens including but not limited to TAC, FOLFOX, TPC, FEC, ADE, FOLFOX-6, EPOCH, CHOP, CMF, CVP, BEP, OFF, FLOX, CVD, TC, FOLFIRI, PCV, FOLFOXIRI, ICE-V, XELOX, and others that are readily appreciated by the skilled clinician in the art.

In some embodiments, the hIL10R binding molecule is administered in combination with BRAF/MEK inhibitors, kinase inhibitors such as sunitinib, PARP inhibitors such as olaparib, EGFR inhibitors such as osimertinib (Ahn, et al. (2016) J Thorac Oncol 11:S115), IDO inhibitors such as epacadostat, and oncolytic viruses such as talimogene laherparepvec (T-VEC).

In some embodiments, a “supplemental agent” is a therapeutic antibody (including bi-specific and tri-specific antibodies which bind to one or more tumor associated antigens including but not limited to bispecific T cell engagers (BITEs), dual affinity retargeting (DART) constructs, and trispecific killer engager (TriKE) constructs).

In some embodiments, the therapeutic antibody is an antibody that binds to at least one tumor antigen selected from the group consisting of HER2 (e.g. trastuzumab, pertuzumab, ado-trastuzumab emtansine), nectin-4 (e.g. enfortumab), CD79 (e.g. polatuzumab vedotin), CTLA4 (e.g. ipilumumab), CD22 (e.g. moxetumomab pasudotox), CCR4 (e.g. magamuizumab), IL23p19 (e.g. tildrakizumab), PDL1 (e.g. durvalumab, avelumab, atezolizumab), IL17a (e.g. ixekizumab), CD38 (e.g. daratumumab), SLAMF7 (e.g. elotuzumab), CD20 (e.g. rituximab, tositumomab, ibritumomab and ofatumumab), CD30 (e.g. brentuximab vedotin), CD33 (e.g. gemtuzumab ozogamicin), CD52 (e.g. alemtuzumab), EpCam, CEA, fpA33, TAG-72, CAIX, PSMA, PSA, folate binding protein, GD2 (e.g. dinuntuximab), GD3, IL6 (e.g. silutxumab) GM2, Ley, VEGF (e.g. bevacizumab), VEGFR, VEGFR2 (e.g. ramucirumab), PDGFR (e.g. olartumumab), EGFR (e.g. cetuximab, panitumumab and necitumumab), ERB2 (e.g. trastuzumab), ERBB3, MET, IGF1R, EPHA3, TRAIL R1, TRAIL R2, RANKL RAP, tenascin, integrin αVβ3, and integrin α4β1.

Examples of antibody therapeutics which are FDA approved and may be used as supplemental agents for use in the treatment of neoplastic disease include those provided in the table below.

| Name | Tradename(s) | Target; format | Indication |
|---|---|---|---|
| [fam]- | Enhertu | HER2; Humanized IgG1 | HER2+ breast cancer |
| trastuzumab | | ADC | |
| deruxtecan | | | |
| Enfortumab | Padcev | Nectin-4; Human IgG1 | Urothelial cancer |
| vedotin | | ADC | |
| Polatuzumab | Polivy | CD79b; Humanized IgG1 | Diffuse large B-cell |
| vedotin | | ADC | lymphoma |
| Cemiplimab | Libtayo | PD-1; Human mAb | Cutaneous squamous cell |
| | | | carcinoma |
| Moxetumomab | Lumoxiti | CD22; Murine IgG1 dsFv | Hairy cell leukemia |
| pasudotox | | immunotoxin | |
| Mogamuizumab | Poteligeo | CCR4; Humanized IgG1 | Cutaneous T cell lymphoma |
| Tildrakizumab | Ilumya | IL23p19; Humanized IgG1 | Plaque psoriasis |
| Ibalizumab | Trogarzo | CD4; Humanized IgG4 | HIV infection |
| Durvalumab | IMFINZI | PD-L1; Human IgG1 | Bladder cancer |
| Inotuzumab | BESPONSA | CD22; Humanized IgG4, | Hematological malignancy |
| ozogamicin | | ADC | |
| Avelumab | Bavencio | PD-L1; Human IgG1 | Merkel cell carcinoma |
| Atezolizumab | Tecentriq | PD-L1; Humanized IgG1 | Bladder cancer |
| Olaratumab | Lartruvo | PDGRFα; Human IgG1 | Soft tissue sarcoma |
| Ixekizumab | Taltz | IL-17a; Humanized IgG4 | Psoriasis |
| Daratumumab | Darzalex | CD38; Human IgG1 | Multiple myeloma |
| Elotuzumab | Empliciti | SLAMF7; Humanized IgG1 | Multiple myeloma |
| Necitumumab | Portrazza | EGFR; Human IgG1 | Non-small cell lung cancer |
| Dinutuximab | Unituxin | GD2; Chimeric IgG1 | Neuroblastoma |
| Nivolumab | Opdivo | PD1; Human IgG4 | Melanoma, non-small cell |
| | | | lung cancer |
| Blinatumomab | Blincyto | CD19, CD3; Murine | Acute lymphoblastic leukemia |
| | | bispecific tandem scFv | |
| Pembrolizumab | Keytruda | PD1; Humanized IgG4 | Melanoma |
| Ramucirumab | Cyramza | VEGFR2; Human IgG1 | Gastric cancer |
| Siltuximab | Sylvant | IL-6; Chimeric IgG1 | Castleman disease |
| Obinutuzumab | Gazyva | CD20; Humanized IgG1; | Chronic lymphocytic |
| | | Glycoengineered | leukemia |
| Ado-trastuzumab | Kadcyla | HER2; Humanized IgG1, | Breast cancer |
| emtansine | | ADC | |
| Pertuzumab | Perjeta | HER2; Humanized IgG1 | Breast Cancer |
| Brentuximab | Adcetris | CD30; Chimeric IgG1, ADC | Hodgkin lymphoma, systemic |
| vedotin | | | anaplastic large cell |
| | | | lymphoma |
| Ipilimumab | Yervoy | CTLA-4; Human IgG1 | Metastatic melanoma |
| Ofatumumab | Arzerra | CD20; Human IgG1 | Chronic lymphocytic |
| | | | leukemia |
| Certolizumab | Cimzia | TNF; Humanized Fab, | Crohn disease |
| pegol | | pegylated | |
| Catumaxomab | Removab | EPCAM/CD3;Rat/mouse | Malignant ascites |
| | | bispecific mAb | |
| Panitumumab | Vectibix | EGFR; Human IgG2 | Colorectal cancer |
| Bevacizumab | Avastin | VEGF; Humanized IgG1 | Colorectal cancer |
| Cetuximab | Erbitux | EGFR; Chimeric IgG1 | Colorectal cancer |
| Tositumomab- | Bexxar | CD20; Murine IgG2a | Non-Hodgkin lymphoma |
| I131 | | | |
| Ibritumomab | Zevalin | CD20; Murine IgG1 | Non-Hodgkin lymphoma |
| tiuxetan | | | |
| Gemtuzumab | Mylotarg | CD33; Humanized IgG4, | Acute myeloid leukemia |
| ozogamicin | | ADC | |
| Trastuzumab | Herceptin | HER2; Humanized IgG1 | Breast cancer |
| Infliximab | Remicade | TNF; Chimeric IgG1 | Crohn disease |
| Rituximab | MabThera, | CD20; Chimeric IgG1 | Non-Hodgkin lymphoma |
| | Rituxan | | |
| Edrecolomab | Panorex | EpCAM; Murine IgG2a | Colorectal cancer |

In some embodiments, where the antibody is a bispecific antibody targeting a first and second tumor antigen such as HER2 and HER3 (abbreviated HER2×HER3), FAP×DR-5 bispecific antibodies, CEA×CD3 bispecific antibodies, CD20×CD3 bispecific antibodies, EGFR-EDV-miR16 trispecific antibodies, gp100×CD3 bispecific antibodies, Ny-eso×CD3 bispecific antibodies, EGFR×cMet bispecific antibodies, BCMA×CD3 bispecific antibodies, EGFR-EDV bispecific antibodies, CLEC12A×CD3 bispecific antibodies, HER2×HER3 bispecific antibodies, Lgr5×EGFR bispecific antibodies, PD1×CTLA-4 bispecific antibodies, CD123×CD3 bispecific antibodies, gpA33×CD3 bispecific antibodies, B7-H3×CD3 bispecific antibodies, LAG-3×PD1 bispecific antibodies, DLL4×VEGF bispecific antibodies, Cadherin-P×CD3 bispecific antibodies, BCMA×CD3 bispecific antibodies, DLL4×VEGF bispecific antibodies, CD20×CD3 bispecific antibodies, Ang-2×VEGF-A bispecific antibodies,

CD20×CD3 bispecific antibodies, CD123×CD3 bispecific antibodies, SSTR2×CD3 bispecific antibodies, PD1×CTLA-4 bispecific antibodies, HER2×HER2 bispecific antibodies, GPC3×CD3 bispecific antibodies, PSMA×CD3 bispecific antibodies, LAG-3×PD-L1 bispecific antibodies, CD38×CD3 bispecific antibodies, HER2×CD3 bispecific antibodies, GD2×CD3 bispecific antibodies, and CD33×CD3 bispecific antibodies. Such therapeutic antibodies may be further conjugated to one or more chemotherapeutic agents (e.g antibody drug conjugates or ADCs) directly or through a linker, especially acid, base or enzymatically labile linkers.

In some embodiments, a supplemental agent is one or more non-pharmacological modalities (e.g., localized radiation therapy or total body radiation therapy or surgery). By way of example, the present disclosure contemplates treatment regimens wherein a radiation phase is preceded or followed by treatment with a treatment regimen comprising an IL10R binding molecule and one or more supplemental agents. In some embodiments, the present disclosure further contemplates the use of an IL10R binding molecule in combination with surgery (e.g. tumor resection). In some embodiments, the present disclosure further contemplates the use of an IL10R binding molecule in combination with bone marrow transplantation, peripheral blood stem cell transplantation or other types of transplantation therapy.

some embodiments, a “supplemental agent” is an immune checkpoint modulator for the treatment and/or prevention neoplastic disease in a subject as well as diseases, disorders or conditions associated with neoplastic disease. The term “immune checkpoint pathway” refers to biological response that is triggered by the binding of a first molecule (e.g. a protein such as PD1) that is expressed on an antigen presenting cell (APC) to a second molecule (e.g. a protein such as PDL1) that is expressed on an immune cell (e.g. a T-cell) which modulates the immune response, either through stimulation (e.g. upregulation of T-cell activity) or inhibition (e.g. downregulation of T-cell activity) of the immune response. The molecules that are involved in the formation of the binding pair that modulate the immune response are commonly referred to as “immune checkpoints.” The biological responses modulated by such immune checkpoint pathways are mediated by intracellular signaling pathways that lead to downstream immune effector pathways, such as cell activation, cytokine production, cell migration, cytotoxic factor secretion, and antibody production. Immune checkpoint pathways are commonly triggered by the binding of a first cell surface expressed molecule to a second cell surface molecule associated with the immune checkpoint pathway (e.g. binding of PD1 to PDL1, CTLA4 to CD28, etc.). The activation of immune checkpoint pathways can lead to stimulation or inhibition of the immune response.

In one embodiment, the immune checkpoint pathway modulator is an antagonist of a negative immune checkpoint pathway that inhibits the binding of PD1 to PDL1 and/or PDL2 (“PD1 pathway inhibitor”). PD1 pathway inhibitors result in the stimulation of a range of favorable immune response such as reversal of T-cell exhaustion, restoration cytokine production, and expansion of antigen-dependent T-cells. PD1 pathway inhibitors have been recognized as effective variety of cancers receiving approval from the USFDA for the treatment of variety of cancers including melanoma, lung cancer, kidney cancer, Hodgkins lymphoma, head and neck cancer, bladder cancer and urothelial cancer.

The term PD1 pathway inhibitors includes monoclonal antibodies that interfere with the binding of PD1 to PDL1 and/or PDL2. Antibody PD1 pathway inhibitors are well known in the art. Examples of commercially available PD1 pathway inhibitors that monoclonal antibodies that interfere with the binding of PD1 to PDL1 and/or PDL2 include nivolumab (Opdivo®, BMS-936558, MDX1106, commercially available from BristolMyers Squibb, Princeton NJ), pembrolizumab (Keytruda®MK-3475, lambrolizumab, commercially available from Merck and Company, Kenilworth NJ), and atezolizumab (Tecentriq®, Genentech/Roche, South San Francisco CA). Additional PD1 pathway inhibitors antibodies are in clinical development including but not limited to durvalumab (MEDI4736, Medimmune/AstraZeneca), pidilizumab (CT-011, CureTech), PDR001 (Novartis), BMS-936559 (MDX1105, BristolMyers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011.

In some embodiments, the methods of the disclosure may include the combination of the administration of an IL10R binding molecules with supplemental agents in the form of cell therapies for the treatment of neoplastic, autoimmune or inflammatory diseases. Examples of cell therapies that are amenable to use in combination with the methods of the present disclosure include but are not limited to engineered T cell products comprising one or more activated CAR-T cells, engineered TCR cells, tumor infiltrating lymphocytes (TILs), engineered Treg cells. As engineered T-cell products are commonly activated ex vivo prior to their administration to the subject and therefore provide upregulated levels of CD25, cell products comprising such activated engineered T cells types are amenable to further support via the administration of an CD25 biased IL10R binding molecule as described herein. Examples of commercially available CAR-T cell products that may be modified to incorporate an orthogonal receptor of the present invention include axicabtagene ciloleucel (marketed as Yescarta® commercially available from Gilead Pharmaceuticals) and tisagenlecleucel (marketed as Kymriah® commercially available from Novartis).

### Prophylactic Applications

In some embodiments where the IL10R binding molecule is used in prophylaxis of disease, the supplementary agent may be a vaccine. The IL10R binding molecule of the present invention may be administered to a subject in combination with vaccines as an adjuvant to enhance the immune response to the vaccine in accordance with the teaching of Doyle, et al U.S. Pat. No. 5,800,819 issued Sep. 1, 1998. Examples of vaccines that may be combined with the IL10R binding molecule of the present invention include are HSV vaccines, Bordetella pertussis, Escherichia coli vaccines, pneumococcal vaccines including multivalent pneumococcal vaccines such as Prevnar® 13, diptheria, tetanus and pertussis vaccines (including combination vaccines such as Pediatrix®) and Pentacel®), varicella vaccines, Haemophilus influenzae type B vaccines, human papilloma virus vaccines such as Garasil®, polio vaccines, Leptospirosis vaccines, combination respiratory vaccine, Moraxella vaccines, and attenuated live or killed virus vaccine products such as bovine respiratory disease vaccine (RSV), multivalent human influenza vaccines such as Fluzone® and Quadravlent Fluzone®), feline leukemia vaccine, transmissible gastroenteritis vaccine, COVID-19 vaccine, and rabies vaccine.

### TABLES

TABLE 1
Amino Acid Abbreviations

| | | |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

TABLE 2
hIL10Ra VHH CDR Amino Acid Sequences

| | CDR1 | | CDR2 | | |
|---|---|---|---|---|---|
| CDR1 AA | SEQ | | SEQ | | CDR1 |
| Seq | ID | CDR2 AA Seq | ID | CDR3 AA Seq | SEQ ID |
| YLYSIDYMA | 1 | VIYTASGATFYPDSVKG | 2 | VRKTDSYLFDAQSFTY | 3 |
| YLYSTNYMA | 4 | VIYTASGATLYTDSVKG | 5 | VRKTDSYLFDAQSFTY | 6 |
| YLYSTNYMA | 7 | VIYTASGATLYTDSVKG | 8 | VRKTDSYLFDAQSFTY | 9 |
| YLYSIDYMA | 10 | VIYTASGATFYPDSVKG | 11 | VRKTDSYLFDAQSFTY | 12 |
| YLYSTNYMA | 13 | AIYTASGATLYSDSNKG | 14 | VRKTGSYLFDAQSFTY | 15 |
| FTYSSYCMG | 16 | SIDSDGSTSYTDSVKG | 17 | DLMSTVVPGFCGFLLSAGMDY | 18 |
| YTFNSNCMG | 19 | TIYTGVGSTYYADSVKG | 20 | EPLSRVYGGSCPTPTFGY | 21 |
| YTYSMYCMG | 22 | QINSDGSTSYADSVKG | 23 | DSRVYGGSWYERLCGPYTYEYNY | 24 |
| YAYSTYCMG | 25 | AIDSGGSTSYADSVKG | 26 | VPPPPDGGSCLFLGPEIKVSKADFRY | 27 |
| YLYSIDYMA | 28 | VIYTASGATFYPDSVKG | 29 | VRKTDSYLFDAQSFTY | 30 |
| YTYSSYCMG | 31 | VIDSDGSTSYADSVKG | 32 | DLGHYRPPCGVLYLGMDY | 33 |
| YTYSSNCMG | 34 | TIYTGGGNTYYADSVKG | 35 | EPLSRVYGGSCPTPTFDY | 36 |
| YSYSSNCMG | 37 | TIHTGGGSTYYADSVKG | 38 | EPLSRLYGGSCPTPTFGY | 39 |
| YTYSSYCMG | 40 | VIDSDGSTSYADSVKG | 41 | DLGHYRPPCGVLYLGMDY | 42 |
| YTYSGYCMG | 43 | VIDSDGSTSYADSVKG | 44 | DLGHYRPPCGVLYLGMDY | 45 |
| YTYSNYCMG | 46 | TIDSDGNTSYADSVKG | 47 | DLGHYRPPCGAYYYGMDY | 48 |
| YSNCSYDMT | 49 | AIHSDGSTRYADSVKG | 50 | DPLHCRAHGGSWYSVRANY | 51 |
| YTYNSNCMG | 52 | TIYTGVGSTYYADSVKG | 53 | EPLSRVYGGSCPTPTFGY | 54 |

TABLE 3
hIL10Rb VHH CDR Amino Acid Sequences

| | CDR1 | | CDR2 | | CDR3 |
|---|---|---|---|---|---|
| CDR1 | SEQ | | SEQ | | SEQ |
| AA Seq | ID | CDR2 AA Seq | ID | CDR3AA Seq | ID |
| YTYSSGCMG | 55 | AINSDGSTSYADSVKG | 56 | EPYCSGGYPRWSVAEFGY | 57 |
| YTYSSYCMG | 58 | AIDSDGSTRYADSVKG | 59 | EPYCSGGYKRTMVAEFGY | 60 |
| YTYNSYCMG | 61 | TIDSDGMTRYADSVKG | 62 | DADCTIAAMTTNP | 63 |
| YLYSIDYMA | 64 | VIYTASGATFYPDSVKG | 65 | VRKTDSYLFDAQSFTY | 66 |
| YTYSSYCMG | 67 | HIDSDGSTTYADSVKG | 68 | DPIPGPGYCDGGPNKY | 69 |
| DLYSTNYVA | 70 | VIYTASGATLYSDSVKG | 71 | VRKTGHYLFDAQSFTY | 72 |
| YTYSSGCMG | 73 | TINSDGSTNYADSVKG | 74 | EPYCSGGYPRWSVAEFGY | 75 |
| YSYSSYCMG | 76 | AIASDGSTSYADSVKG | 77 | EPWCTGGYSRLTPAEYGY | 78 |
| YTYSSGCMG | 79 | TINSDGSTNYADSVKG | 80 | EPYCSGGYPRWSVAEFGY | 81 |
| YTYSSYCMG | 82 | HIDSDGSTTYADSVKG | 83 | DPIPGPGYCDGGPNKY | 84 |
| YTYSSYCMG | 85 | AIDSDGSTRYADSVKG | 86 | EPYCSGGYKRTMVAEFGY | 87 |
| YTYSSYCMG | 88 | HIDSDGSTSYADSVKG | 89 | DPIPGPGYCDGGPNKY | 90 |
| YTYSSYCMG | 91 | HIDSDGSTSYADSVKG | 92 | DPIPGPGYCDGGPNNY | 93 |
| YTYSSGCMG | 94 | TINSDGSTNYADSVKG | 95 | EPYCSGGYPRWSVAEFGY | 96 |
| YTASVNYMG | 97 | TIFTGAGTTYYANSVKG | 98 | DFRGGLLYRPAYEYTY | 99 |
| YTHSSYCMG | 100 | AIDVDGSTTYADSVKG | 101 | EFADCSSNYFLPPGAVRY | 102 |
| YTASVNYMG | 103 | TIFTGAGTTYYANSVKG | 104 | DFRGGLLYRPAYEYTY | 105 |
| DTYSSYCMG | 106 | FIDSDGSTRYADSVEG | 107 | EPYCSGGYHRKEMAEFGY | 108 |
| YTYSSYCMG | 109 | HIDSDGSTSYADSVKG | 110 | DPIPGPGYCDGGPNKY | 111 |
| YTYSSYCMG | 112 | HIDSDGSTTYADSVKG | 113 | DPIPGPGYCDGGPNKY | 114 |
| YTASNNCMG | 115 | VIFTGAGTSYYDSSVG | 116 | EDDCTLLLMTPNPDDQ | 117 |
| YTDSRYCMG | 118 | HIDSDGSTSYADSVKG | 119 | DPIPGPGYCDGGPNKY | 120 |
| YTYSSYCMG | 121 | AIDSDGSTRYADSVKG | 122 | EPYCSGGYKRTMVAEFGF | 123 |
| YTYSSYCMG | 124 | HIDSDGSTTYADSVKG | 125 | DPIPGPGYCDGGPNNY | 126 |
| YTYSSYCMG | 127 | HIDSDGSTTYADSVKG | 128 | DPIPGPGYCDGGPNNY | 129 |
| YSYSSYCMG | 130 | TIDSDGMTRYADSVKG | 131 | PLYDCDSGAVGRNPPY | 132 |
| YTYLRGCMG | 133 | VMDVVGDRRSYIDSVKG | 134 | GPNCVGWRSGLDY | 135 |

TABLE 4
ANTI MOUSE mIL10RB VHHs and CDRs Amino Acid (AA) Sequences

| | CDR1 | | CDR2 | | |
|---|---|---|---|---|---|
| CDR1 | SEQ | | SEQ | | CDR3 |
| AA Seq | ID | CDR2 AA Seq | ID | CDR3 AA Seq | SEQ ID |
| YTASSICMG | 136 | VITTAASGTYYADSVNG | 137 | TRRGGDCLDPLQTPAVNT | 138 |
| DTYSRKYIA | 139 | VMYTPGSATYYTDTVMG | 140 | KASGSMFNFRDYTY | 141 |
| YASCSRAMR | 142 | YIDGVGSTGYADSV | 143 | GCRADGSNSLDNY | 144 |
| YTYNRRFMG | 145 | IIYTPNSSTFYADSVTG | 146 | ARIASMTELSVRDMDY | 147 |
| YIALNACMA | 148 | TIVTDGSRTYYADSVKG | 149 | DRRCPVSRAPYEYELRY | 150 |
| YTYNGKCMA | 151 | GIYTGGSSTYYADSVKG | 152 | SRSCSDLRRRSIAY | 153 |

TABLE 5
hIL10Ra VHH Amino Acid Sequences

| Name | VHH Amino Acid Sequence | SEQ ID |
|---|---|---|
| hIL10Ra VHH1 | QVQLQESGGGSIQAGGSLRLSCAASRYLYSIDYMAWFRQSPGKERE | 154 |
| | PVAVIYTASGATFYPDSVKGRFTISQDNAKMTVYLQMNSLKSEDTA | |
| | MYYCAAVRKTDSYLFDAQSFTYWGQGTQVTVSS ( | |
| hIL10Ra VHH2 | QVQLQESGGGSVQAGGSLRLSCVASRYLYSTNYMAWFRQSPGKERE | 155 |
| | AVAVIYTASGATLYTDSVKGRFTISQDNAKMTVYLQMNRLKSEDTA | |
| | MYYCAAVRKTDSYLFDAQSFTYWGQGTQVTVSS | |
| hIL10Ra VHH3 | QVQLQESGGGSIQAGGSLRLSCVASRYLYSTNYMAWFRQSPGKERE | 156 |
| | AVAVIYTASGATLYTDSVKGRFTISQDNAKMTVYLQMNRLKSEDTA | |
| | MYYCAAVRKTDSYLFDAQSFTYWGQGTQVTVSS | |
| hIL10Ra VHH4 | QVQLQESGGGSIQAGGSLRLSCAASRYLYSIDYMAWFRQSPGKERE | 157 |
| | PAAVIYTASGATFYPDSVKGRFTISQDNAKMTVYLQMNSLKSEDTA | |
| | MYYCAAVRKTDSYLFDAQSFTYWGQGTQVTVSS | |
| hIL10Ra VHH5 | QVQLQESGGGSIQAGGSLRLSCVASKYLYSTNYMAWFRQSPGKERE | 158 |
| | AVAAIYTASGATLYSDSNKGRFTISQDNAKMTVYLQMNSLKSEDTA | |
| | MYYCAAVRKTGSYLFDAQSFTYWGQGTQVTVSS | |
| hIL10Ra VHH6 | QVQLQESGGGSVQAGGSLRLSCAASRFTYSSYCMGWFRQAPGKERE | 159 |
| | GVASIDSDGSTSYTDSVKGRFTISKDNAKNTLYLQMNSLKPEDTAM | |
| | YYCALDLMSTVVPGFCGFLLSAGMDYWGKGTQVTVSS | |
| hIL10Ra VHH7 | QVQLQESGGGSVQAGGSLRLSCAVSGYTFNSNCMGWFRQAPGKERE | 160 |
| | GVATIYTGVGSTYYADSVKGRFTISQDNAKNTVYLQMNSLKPEDTA | |
| | MYYCAAEPLSRVYGGSCPTPTFGYWGQGTQVTVSS | |
| hIL10Ra VHH8 | QVQLQESGGGSVQAGGSLRLSCAASGYTYSMYCMGWFRQAPGKERE | 161 |
| | GVAQINSDGSTSYADSVKGRFTISKDNAKNTLYLQMNSLKPEDTAM | |
| | YYCAADSRVYGGSWYERLCGPYTYEYNYWGQGTQVTVSS | |
| hIL10Ra VHH9 | QVQLQESGGGSVQAGGSLRLSCAVSGYAYSTYCMGWFRQAPGKERE | 162 |
| | GVAAIDSGGSTSYADSVKGRFTISKDNAKNTLYLRMNSLKPEDTAM | |
| | YYCAAVPPPPDGGSCLFLGPEIKVSKADFRYWGQGTQVTVSS | |
| hIL10Ra VHH10 | QVQLQESGGGSVQAGGSLRLSCAASRYLYSIDYMAWFRQSPGKERE | 163 |
| | PVAVIYTASGATFYPDSVKGRFTISQDNAKMTVYLQMNSLKSEDTA | |
| | MYYCAAVRKTDSYLFDAQSFTYWGQGTQVTVSS | |
| hIL10Ra VHH11 | QVQLQESGGGSVQAGGSLRLSCGASRYTYSSYCMGWFRQAPGKERE | 164 |
| | GVAVIDSDGSTSYADSVKGRFTISKDNGKNTLYLQMNSLKPEDTAM | |
| | YYCAADLGHYRPPCGVLYLGMDYWGKGTQVTVSS | |
| hIL10Ra VHH12 | QVQLQESGGGSVQAGGSLRLSCTVSGYTYSSNCMGWFRQAPGKERE | 165 |
| | GVATIYTGGGNTYYADSVKGRFTISQDNAKNTVYLQMNNLKPEDTA | |
| | MYYCAAEPLSRVYGGSCPTPTFDYWGQGTQVTVSS | |
| hIL10Ra VHH13 | QVQLQESGGGSVQAGGSLRLSCAVSGYSYSSNCMGWFRQAPGKERE | 166 |
| | GVATIHTGGGSTYYADSVKGRFTISQDNAKNTVYLQMNSLKPEDTA | |
| | MYYCAAEPLSRLYGGSCPTPTFGYWGQGTQVTVSS | |
| hIL10Ra VHH14 | QVQLQESGGGSVQAGGSLRLSCGASGYTYSSYCMGWFRQVPGKERE | 167 |
| | GVAVIDSDGSTSYADSVKGRFTISKDNGKNTLYLQMNSLKPEDTAM | |
| | YYCAADLGHYRPPCGVLYLGMDYWGKGTQVTVSS | |
| hIL10Ra VHH15 | QVQLQESGGGSVQAGGSLRLSCGASGYTYSGYCMGWFRQAPGKERE | 168 |
| | GVAVIDSDGSTSYADSVKGRFTISKDNGKNTLYLQMNSLKPEDTAM | |
| | YYCAADLGHYRPPCGVLYLGMDYWGKGTQVTVSS | |
| hIL10Ra VHH16 | QVQLQESGGGSVQAGGSLRLACAASRYTYSNYCMGWFRQAPGKERE | 169 |
| | GVATIDSDGNTSYADSVKGRFTISRDNAKNTLYLQMNSLKPGDTAM | |
| | YYCAADLGHYRPPCGAYYYGMDYWGKGTQVTVSS | |
| hIL10Ra VHH17 | QVQLQESGGGSVQAGGSLRLSCAASGYSNCSYDMTWYRQAPGKERE | 170 |
| | FVSAIHSDGSTRYADSVKGRFFISQDNAKNTVYLQMNSLKPEDTAM | |
| | YYCKTDPLHCRAHGGSWYSVRANYWGQGTQVTVSS | |
| hIL10Ra VHH18 | QVQLQESGGGSVQAGGSLRLSCAVSGYTYNSNCMGWFRQAPGKERE | 171 |
| | GVATIYTGVGSTYYADSVKGRFTISQDNAKNTVYLQMNSLKPEDTA | |
| | MYYCAAEPLSRVYGGSCPTPTFGYWGQGTQVTVSS | |

TABLE 6
ANTI HUMAN hIL10Rb VHH Sequences

| Name | VHH AA Sequence (CDRs Underlined) | SEQ ID |
|---|---|---|
| IL10Rb | QVQLQESGGGSVQAGGSLRLSCAASGYTYSSGCMGWFRQAPGKEREAVA | 172 |
| VHH-1 | AINSDGSTSYADSVKGRFTISKDNAKNTLYLQMNSLKPEDTAMYYCAAE | |
| | PYCSGGYPRWSVAEFGYWGQGTQVTVSS | |
| hIL10Rb | QVQLQESGGGSVQAGGSLRLSCAASGYTYSSYCMGWFRQAPGKEREGVA | 173 |
| VHH-2 | AIDSDGSTRYADSVKGRFTISKDNAKNTLYLQMNSLKPEDTAMYYCAAE | |
| | PYCSGGYKRTMVAEFGYWGQGTQVTVSS | |
| hIL10Rb | QVQLQESGGGSVQAGGSLRLSCAASRYTYNSYCMGWFRQAPGKEREGVA | 174 |
| VHH-3 | TIDSDGMTRYADSVKGRFTISKDNAKNTLYLQMNSLKPEDTAMYYCAAD | |
| | ADCTIAAMTTNPLGQGTQVTVSS | |
| hIL10Rb | QVQLQESGGGSIQAGGSLRLSCAASRYLYSIDYMAWFRQSPGKEREPVA | 175 |
| VHH-4 | VIYTASGATFYPDSVKGRFTISQDNAKMTVYLQMNSLKSEDTAMYYCAA | |
| | VRKTDSYLFDAQSFTYWGQGTQVTVSS | |
| hIL10Rb | QVQLQESGGGLVQPGGSLRLSCAASGYTYSSYCMGWFRQAPGKEREGVA | 176 |
| VHH-5 | HIDSDGSTTYADSVKGRFTISKDNAKNTLYLQMNSLKPEDTAMYYCAAD | |
| | PIPGPGYCDGGPNKYWGQGTQVTVSS | |
| hIL10Rb | QVQLQESGGGSIQAGGSLTLSCAASRDLYSTNYVAWFRQSPGKEREAVA | 177 |
| VHH-6 | VIYTASGATLYSDSVKGRFTISQDNAKMTVYLQMNSLKSEDTAMYYCAA | |
| | VRKTGHYLFDAQSFTYWGQGTQVTVSS | |
| hIL10Rb | QVQLQESGGGSVQAGGSLRLSCAASGYTYSSGCMGWFRQAPGKEREGVA | 178 |
| VHH-7 | TINSDGSTNYADSVKGRFTISKDNAKNTLYLQMNSLKPEDTAMYYCAAE | |
| | PYCSGGYPRWSVAEFGYWGQGTQVTVSS | |
| hIL10Rb | QVQLQESGGGSVQAGGSLRLSCAASGYSYSSYCMGWFRQAPGKEREGVA | 179 |
| VHH-8 | AIASDGSTSYADSVKGRFAISKDNAKNTLYLQMASLKPEDTAMYYCAAE | |
| | PWCTGGYSRLTPAEYGYWGQGTQVTVSS | |
| hIL10Rb | QVQLQESGGGLVQPGGSLRLSCAASGYTYSSGCMGWFRQAPGKEREGVA | 180 |
| VHH-9 | TINSDGSTNYADSVKGRFTISKDNAKNTLYLQMNSLKPEDTAMYYCAAE | |
| | PYCSGGYPRWSVAEFGYWGQGTQVTVSS | |
| hIL10Rb | QVQLQESGGGLVQPGGSLRLSCAASGYTYSSYCMGWFRQAPGKEREGVA | 181 |
| VHH-10 | HIDSDGSTTYADSVKGRFAISKDNAKNTLYLQMNSLKPEDTAMYYCAAD | |
| | PIPGPGYCDGGPNKYWGQGTQVTVSS | |
| hIL10Rb | QVQLQESGGGSVQAGGSLRLSCAASGYTYSSYCMGWFRQAPGKGREGVA | 182 |
| VHH-11 | AIDSDGSTRYADSVKGRFTISKDNAKNTLYLQMNSLKPEDTAMYYCAAE | |
| | PYCSGGYKRTMVAEFGYWGQGTQVTVSS | |
| hIL10Rb | QVQLQESGGGSVQAGGSLRLSCAASGYTYSSYCMGWFRQAPGKEREGVA | 183 |
| VHH-12 | HIDSDGSTSYADSVKGRFTISKDNAKNTLYLQMNSLKPEDTAMYYCAAD | |
| | PIPGPGYCDGGPNKYWGQGTQVTVSS | |
| hIL10Rb | QVQLQESGGGSVQAGGSLRLSCAASGYTYSSYCMGWFRQAPGKEREGVA | 184 |
| VHH-13 | HIDSDGSTSYADSVKGRFTISKDNAKNTLYLQMNSLKPEDTAMYYCAAD | |
| | PIPGPGYCDGGPNNYWGQGTQVTVSS | |
| hIL10Rb | QVQLQESGGGSVQAGGSLRLSCAASGYTYSSGCMGWFRQAPGKEREGVA | 185 |
| VHH-14 | TINSDGSTNYADSVKGRFTISKDNAKNTLYLQMNSLKPEDTGMYYCAAE | |
| | PYCSGGYPRWSVAEFGYWGQGTQVTVSS | |
| hIL10Rb | QVQLQESGGGSVQAGGSLRLSCTVSRYTASVNYMGWFRQAPGKEREGVA | 186 |
| VHH-15 | TIFTGAGTTYYANSVKGRFTISRDNAKNTAYLQMNSLKPEDTAIYYCAV | |
| | DFRGGLLYRPAYEYTYRGQGTQVTVSS | |
| hIL10Rb | QVQLQESGGGSVEAGGSLRLSCAASGYTHSSYCMGWFRQAPGKEREGVA | 187 |
| VHH-16 | AIDVDGSTTYADSVKGRFTISKDNAKNTLYLQMNSLKPEDTGMYYCAAE | |
| | FADCSSNYFLPPGAVRYWGQGTQVTVSS | |
| hIL10Rb | QVQLQESGGGSVQAGGSLRLSCTVSRYTASVNYMGWFRQAPGKEREGVA | 188 |
| VHH-17 | TIFTGAGTTYYANSVKGRFTISRDNAKNTAYLQMNSLKPEDTAMYYCAV | |
| | DFRGGLLYRPAYEYTYRGQGTQVTVSS | |
| hIL10Rb | QVQLQESGGGSVQAGGSLRLSCAASGDTYSSYCMGWFRQAPGKEREGVA | 189 |
| VHH-18 | FIDSDGSTRYADSVEGRFTISKDNAKNTLYLQMNSLKPEDTAMYYCAAE | |
| | PYCSGGYHRKEMAEFGYWGQGTQVTVSS | |
| hIL10Rb | QVQLQESGGGSVQAGGSLRLSCAASGYTYSSYCMGWFRQAPGKEREGVA | 190 |
| VHH-19 | HIDSDGSTSYADSVKGRFTISKDNAKNTLYLQMNSLKPEDTAMYYCAAD | |
| | PIPGPGYCDGGPNKYRGQGTQVTVSS | |
| hIL10Rb | QVQLQESGGGSVQAGGSLRLSCAASGYTYSSYCMGWFRQAPGKEREGVA | 191 |
| VHH-20 | HIDSDGSTTYADSVKGRFAISKDNAKNTLYLQMNSLKPEDTAMYYCAAD | |
| | PIPGPGYCDGGPNKYWGQGTQVTVSS | |
| hIL10Rb | QVQLQESGGGSVQAGGSLRLSCTGSGYTASNNCMGWFRQAPGKEREGVA | 192 |
| VHH-21 | VIFTGAGTSYYDSSVGRLFISSQDAASTLDQLLMSLLPDDTAVMYCGAE | |
| | DDCTLLLMTPNPDDQWSRLSVSS | |
| hIL10Rb | QVQLQESGGGSVQAGGSLRLSCAASGYTDSRYCMGWFRKAPGKEREGVA | 193 |
| VHH-22 | HIDSDGSTSYADSVKGRFTISKDNAKNTLYLQMNSLKPEDTAMYYCAAD | |
| | PIPGPGYCDGGPNKYWGQGTQVTVSS | |
| hIL10Rb | QVQLQESGGGSVQAGGSLRLSCAASGYTYSSYCMGWFRQAPGKEREGVA | 194 |
| VHH-23 | AIDSDGSTRYADSVKGRFTISKDNAKKILYLQMNSLKVEDTAMYYCAAE | |
| | PYCSGGYKRTMVAEFGFWGQGTQVTVSS | |
| hIL10Rb | QVQLQESGGGSVQAGGSLKLSCAASGYTYSSYCMGWFRQAPGKEREGVA | 195 |
| VHH-24 | HIDSDGSTTYADSVKGRFTISKDNAKNTLYLQMNSLKPEDTAMYYCAAD | |
| | PIPGPGYCDGGPNNYWGQGTQVTVSS | |
| hIL10Rb | QVQLQESGGGSVQAGGSLRLSCAASGYTYSSYCMGWFRQAPGKEREGIA | 196 |
| VHH-25 | HIDSDGSTTYADSVKGRFTISKDNAKNTLYLQMNSLKPEDTAMYYCAAD | |
| | PIPGPGYCDGGPNNYWGQGTQVTVSS | |
| hIL10Rb | QVQLQESGGGSVQAGGSLRLSCAASGYSYSSYCMGWFRQAPGKEREGVA | 197 |
| VHH-26 | TIDSDGMTRYADSVKGRFTISKDNAKNTLYLQMNSLKPEDTAMYYCAAP | |
| | LYDCDSGAVGRNPPYWGQGTQVTVSS | |
| hIL10Rb | QVQLQESGGGSVQTGGSLRLSCAASGYTYLRGCMGWFRQAPGKEREGVA | 198 |
| VHH-27 | VMDVVGDRRSYIDSVKGRFTISRDNAANSVYLQMDNLKPEDTAMYYCTA | |
| | GPNCVGWRSGLDYWGQGTQVTVSS | |

TABLE 7
ANTI MOUSE mIL10RB VHHs and CDRs
Amino Acid (AA) Sequences

| | | SEQ |
|---|---|---|
| Name | VHH AA Sequence | ID |
| DR1322 | QVQLQESGGGSVQAGGALRLSCTASGYTASSICMGWFRQ | 199 |
| | APGKERERVAVITTAASGTYYADSVNGRFSISQNNAKNT | |
| | VYLQMNSLKPDDTAMYYCAATRRGGDCLDPLQTPAYNTW | |
| | GQGTQVTVSS | |
| DR1323 | QVQLQESGGGSVQAGGSLRLSCVASGDTYSRKYIAWVRQ | 200 |
| | VPGKEREGVAVMYTPGSATYYTDTVMGRFTISQDNAKNT | |
| | VYLQMNSLKPEDTAMYFCAAKASGSMFNFRDYTYWGQGT | |
| | QVTVSS | |
| DR1324 | QVQLQESGGGSVQAGGSLRLSCATSGYASCSRAMRWYRQ | 201 |
| | APGKEREFVAYIDGVGSTGYADSVKGRFTISQDNAKYTA | |
| | YLQMNSLKPEDTAMYYCNRGCRADGSNSLDNYWGQGTQV | |
| | TVSS | |
| DR1325 | QVQLQESGGGSVQAGGSLRLSCAASGYTYNRRFMGWFRQ | 202 |
| | APGKEREGLAIIYTPNSSTFYADSVTGRFTISQDSARNT | |
| | VYLQMNSLKPEDTAMYYCAAARIASMTELSVRDMDYWGK | |
| | GTQVTVSS | |
| DR1326 | QVQLQESGGGSVQAGGSLRLSCTASRYIALNACMAWIRQ | 203 |
| | APGSEREVVATIVTDGSRTYYADSVKGRFTISQDNAKNT | |
| | MYLQMNGLKPEDTAMYYCAADRRCPVSRAPYEYELRYWG | |
| | QGTQVTVSS | |
| DR1327 | QVQLQESGGGSVQAGGSLRLSCAASGYTYNGKCMAWFRQ | 204 |
| | APGKEREVVAGIYTGGSSTYYADSVKGRFTISQDNAKNT | |
| | VYLQMDSLKPEDTAMYYCATSRSCSDLRRRSIAYWGQGT | |
| | QVTVSS | |

TABLE 8
hIL10Ra VHH DNA Sequences

| VHH | | SEQ |
|---|---|---|
| Name | Sequence | ID |
| hIL10Ra | CAGGTTCAGCTTCAGGAGTCCGGTGGAGGCTCCATCCAGGCCGGGGGCT | 205 |
| VHH1 | CTCTCCGCCTGTCTTGCGCCGCTTCCAGATACCTCTACAGTATCGACTA | |
| | CATGGCTTGGTTTCGTCAGAGCCCAGGAAAAGAGCGGGAACCCGTGGCA | |
| | GTAATCTACACTGCCTCAGGTGCCACATTTTACCCCGACTCTGTCAAGG | |
| | GCAGGTTCACCATCTCTCAGGATAATGCCAAGATGACAGTGTACTTGCA | |
| | GATGAACTCCCTGAAATCTGAGGATACCGCTATGTATTACTGTGCCGCA | |
| | GTGCGCAAGACCGATTCTTACCTGTTCGACGCTCAGAGTTTTACCTACT | |
| | GGGGCCAGGGCACTCAGGTCACCGTCAGCAGC | |
| hIL10Ra | CAGGTGCAGTTGCAGGAGTCCGGCGGGGGTTCCGTGCAAGCAGGCGGAT | 206 |
| VHH2 | CTCTGCGCCTGTCCTGCGTGGCCTCTCGTTATTTGTATAGCACCAACTA | |
| | CATGGCTTGGTTCCGTCAGTCCCCAGGCAAAGAGCGCGAAGCCGTAGCC | |
| | GTAATCTATACGGCCTCTGGGGCAACACTCTATACCGACTCAGTGAAGG | |
| | GACGCTTCACGATTTCCCAAGACAATGCAAAGATGACCGTGTACTTGCA | |
| | GATGAACCGCCTGAAGAGCGAGGACACGGCTATGTATTACTGCGCAGCC | |
| | GTGCGCAAGACCGACTCCTACTTGTTTGACGCTCAGTCCTTCACTTATT | |
| | GGGGCCAGGGTACACAGGTCACCGTGAGCAGT | |
| hIL10Ra | CAAGTACAGCTCCAGGAGAGCGGCGGTGGATCTATCCAAGCAGGGGGTA | 207 |
| VHH3 | GCCTTAGGTTGTCCTGTGTGGCGTCCAGATACCTGTATAGCACGAACTA | |
| | CATGGCATGGTTCAGACAGTCCCCAGGCAAGGAACGCGAGGCAGTCGCC | |
| | GTTATTTACACTGCATCTGGGGCCACCCTCTATACGGACAGCGTGAAGG | |
| | GAAGGTTTACAATCTCCCAGGACAACGCGAAGATGACCGTGTACCTTCA | |
| | GATGAACCGCCTGAAGTCCGAGGACACCGCCATGTATTACTGTGCAGCG | |
| | GTGCGCAAGACCGACAGCTATCTGTTCGACGCGCAGTCATTCACTTATT | |
| | GGGGCCAAGGAACCCAAGTGACCGTCAGCTCA | |
| hIL10Ra | CAGGTGCAGCTCCAAGAGTCCGGGGGAGGCTCTATCCAGGCGGGAGGCA | 208 |
| VHH4 | GTCTGCGCTTGTCCTGCGCCGCAAGTCGTTATCTGTACTCCATTGATTA | |
| | CATGGCATGGTTCCGCCAGTCCCCAGGTAAGGAACGTGAACCTGCCGCT | |
| | GTGATCTACACCGCTTCTGGAGCAACCTTTTATCCTGATAGCGTTAAGG | |
| | GTCGCTTCACCATCTCTCAGGATAACGCCAAAATGACAGTGTACCTCCA | |
| | GATGAACAGCCTGAAGTCTGAGGACACTGCCATGTACTATTGTGCGGCT | |
| | GTGCGCAAGACCGACTCCTATCTGTTTGATGCACAGAGCTTTACCTATT | |
| | GGGGTCAGGGCACCCAGGTGACTGTGTCTAGC | |
| hIL10Ra | CAGGTCCAGTTGCAGGAGTCCGGTGGAGGTTCCATCCAGGCGGGTGGGT | 209 |
| VHH5 | CCCTTCGTCTCTCCTGCGTGGCCTCTAAGTACCTGTATTCAACCAACTA | |
| | CATGGCATGGTTCAGACAGTCTCCCGGCAAAGAGCGTGAGGCAGTGGCC | |
| | GCGATCTATACAGCTTCTGGGGCCACCCTGTACTCTGATTCCAATAAGG | |
| | GAAGGTTCACTATCTCACAGGATAACGCCAAAATGACCGTCTACCTTCA | |
| | GATGAACAGCCTCAAGTCTGAAGACACGGCAATGTATTACTGTGCAGCC | |
| | GTGCGCAAAACTGGGAGCTACCTGTTTGACGCTCAGTCTTTCACTTATT | |
| | GGGGCCAGGGTACGCAGGTGACAGTCTCTTCT | |
| hIL10Ra | CAGGTGCAACTCCAGGAGAGCGGAGGCGGTTCTGTTCAGGCAGGAGGTT | 210 |
| VHH6 | CCCTGAGACTGTCCTGTGCCGCGTCTCGCTTTACGTATTCATCCTACTG | |
| | CATGGGATGGTTCAGACAAGCGCCGGGGAAAGAAAGGGAAGGCGTGGCC | |
| | TCCATTGACTCCGACGGCTCAACTTCATACACTGATAGCGTGAAAGGCC | |
| | GGTTCACCATCTCTAAGGACAACGCGAAGAACACCCTGTATCTCCAGAT | |
| | GAACAGCCTCAAGCCTGAGGATACTGCCATGTACTATTGCGCACTCGAC | |
| | CTGATGTCTACTGTGGTCCCAGGCTTCTGCGGGTTCCTGCTCTCTGCTG | |
| | GCATGGACTACTGGGGGAAGGGCACTCAGGTAACGGTTAGCTCC | |
| hIL10Ra | CAGGTGCAGCTTCAGGAATCTGGCGGGGGCTCCGTGCAGGCCGGGGGCT | 211 |
| VHH7 | CCCTCAGACTTTCCTGTGCCGTCTCCGGTTACACATTTAACAGTAACTG | |
| | TATGGGCTGGTTCCGCCAGGCACCAGGCAAGGAGAGGGAAGGTGTGGCC | |
| | ACAATCTATACTGGTGTTGGGAGTACGTACTATGCTGATTCCGTGAAAG | |
| | GTCGCTTCACAATTTCCCAGGACAACGCGAAGAACACTGTGTACTTGCA | |
| | GATGAATAGCCTGAAGCCTGAAGATACCGCAATGTATTACTGCGCTGCC | |
| | GAGCCACTCTCCCGCGTATATGGTGGAAGTTGCCCCACCCCCACTTTCG | |
| | GTTACTGGGGCCAGGGCACTCAAGTGACCGTGTCCTCT | |
| hIL10Ra | CAGGTTCAGCTTCAGGAGTCTGGGGGCGGTTCAGTGCAGGCTGGCGGTT | 212 |
| VHH8 | CTCTCCGCCTGTCCTGCGCTGCCAGCGGCTATACTTACAGCATGTACTG | |
| | CATGGGCTGGTTCCGGCAAGCCCCCGGCAAAGAGCGTGAGGGCGTCGCT | |
| | CAAATCAACAGCGACGGGTCAACCAGCTACGCCGATTCTGTCAAGGGCA | |
| | GATTTACTATCAGCAAGGACAACGCCAAAAACACACTGTACCTCCAGAT | |
| | GAACTCTTTGAAGCCTGAGGACACCGCGATGTATTACTGCGCCGCTGAC | |
| | AGCCGCGTGTACGGTGGCAGCTGGTATGAGAGGCTGTGCGGCCCGTACA | |
| | CCTACGAGTACAACTATTGGGGACAGGGCACGCAGGTGACAGTTAGCTC | |
| | C | |
| hIL10Ra | CAGGTGCAACTGCAAGAGAGTGGCGGAGGCTCCGTCCAGGCTGGAGGTT | 213 |
| VHH9 | CCCTGCGGCTGTCTTGCGCCGTCAGCGGCTACGCATATTCCACTTACTG | |
| | TATGGGTTGGTTCCGCCAGGCCCCTGGAAAGGAACGCGAGGGTGTTGCC | |
| | GCTATTGATAGCGGAGGCTCCACATCCTATGCGGACTCCGTGAAAGGTC | |
| | GTTTCACCATCTCCAAGGATAACGCCAAGAACACTCTGTACCTGCGCAT | |
| | GAACTCTCTGAAGCCTGAGGACACTGCCATGTATTACTGCGCCGCTGTG | |
| | CCCCCTCCACCCGACGGGGGCTCTTGTCTGTTTCTTGGCCCGGAGATCA | |
| | AGGTGTCCAAGGCTGATTTCCGTTATTGGGGCCAGGGAACTCAAGTCAC | |
| | CGTGTCTTCC | |
| hIL10Ra | CAGGTCCAGCTCCAGGAGTCCGGTGGAGGCTCCGTTCAGGCCGGTGGCA | 214 |
| VHH10 | GCTTGCGTCTGAGCTGCGCGGCTTCAAGATACCTGTACTCCATTGATTA | |
| | CATGGCATGGTTCCGTCAGTCTCCTGGCAAGGAGCGCGAGCCCGTCGCT | |
| | GTGATCTATACCGCCAGCGGAGCCACGTTCTACCCTGATTCCGTCAAGG | |
| | GCCGCTTCACCATTAGCCAAGACAACGCTAAGATGACGGTGTACCTCCA | |
| | AATGAATAGCCTGAAAAGCGAGGACACAGCGATGTATTACTGCGCCGCT | |
| | GTTAGGAAAACTGATAGTTACCTGTTCGATGCACAGTCTTTCACTTACT | |
| | GGGGGCAGGGCACCCAAGTTACCGTCTCCTCT | |
| hIL10Ra | CAGGTGCAGCTCCAGGAATCTGGAGGGGGCAGTGTGCAGGCCGGGGGCT | 215 |
| VHH11 | CCCTGCGCTTGAGCTGTGGAGCCAGCCGCTACACGTATTCCAGTTACTG | |
| | TATGGGCTGGTTCAGACAAGCTCCGGGTAAGGAGAGAGAGGGAGTTGCC | |
| | GTAATTGATTCTGACGGGTCCACTAGCTATGCGGATTCAGTCAAGGGCC | |
| | GGTTCACCATCAGCAAGGACAATGGTAAGAACACACTGTACCTGCAAAT | |
| | GAACAGCCTGAAGCCCGAGGACACCGCCATGTACTATTGTGCCGCTGAT | |
| | CTCGGACATTACCGCCCTCCCTGCGGTGTGCTCTATCTCGGGATGGACT | |
| | ATTGGGGTAAGGGCACCCAGGTGACCGTGTCCTCT | |
| hIL10Ra | CAGGTGCAGCTCCAGGAAAGCGGCGGGGGTAGCGTTCAAGCAGGTGGGT | 216 |
| VHH12 | CCCTGCGCTTGAGCTGTACTGTGTCCGGCTACACCTACTCAAGCAACTG | |
| | CATGGGATGGTTCCGTCAGGCCCCTGGCAAGGAACGCGAAGGCGTGGCT | |
| | ACTATCTACACCGGCGGTGGCAACACTTATTACGCCGACTCCGTTAAGG | |
| | GGCGTTTCACTATCAGCCAAGACAACGCCAAGAACACCGTGTATCTGCA | |
| | AATGAATAACCTGAAGCCTGAAGACACCGCCATGTATTACTGTGCTGCC | |
| | GAGCCCCTTTCCCGCGTTTACGGCGGTTCTTGTCCTACCCCTACCTTTG | |
| | ACTACTGGGGTCAGGGAACACAGGTGACAGTGTCCAGT | |
| hIL10Ra | CAAGTCCAACTCCAGGAATCTGGGGGAGGCTCCGTACAGGCTGGCGGTT | 217 |
| VHH13 | CCCTTCGTCTGTCCTGTGCTGTGTCAGGGTACTCCTACTCCAGTAACTG | |
| | TATGGGCTGGTTCCGGCAAGCCCCCGGAAAGGAGCGCGAGGGCGTGGCT | |
| | ACCATCCACACAGGGGGCGGTTCCACATATTACGCCGATAGTGTCAAGG | |
| | GCCGCTTCACCATTAGTCAGGACAACGCCAAGAATACCGTTTACCTTCA | |
| | AATGAACTCTTTGAAACCTGAGGACACTGCGATGTATTACTGTGCGGCA | |
| | GAGCCTTTGTCCCGCCTGTACGGGGGATCTTGTCCGACCCCGACTTTCG | |
| | GGTACTGGGGACAGGGCACCCAGGTGACAGTGTCCTCC | |
| hIL10Ra | CAGGTGCAGTTGCAGGAAAGCGGGGGTGGCAGCGTCCAAGCCGGTGGCA | 218 |
| VHH14 | GCCTGCGTCTGTCCTGCGGTGCCTCCGGCTATACTTACTCCAGCTATTG | |
| | CATGGGTTGGTTCCGCCAAGTGCCAGGAAAGGAGCGTGAGGGGGTGGCT | |
| | GTAATTGATTCAGATGGGTCAACAAGCTACGCTGACAGCGTTAAAGGTC | |
| | GCTTCACCATCAGTAAGGACAACGGCAAGAACACCCTCTACCTGCAAAT | |
| | GAACTCCCTGAAGCCGGAGGATACCGCAATGTATTACTGTGCCGCTGAC | |
| | TTGGGACACTACCGCCCTCCGTGCGGTGTGCTTTATCTGGGCATGGATT | |
| | ACTGGGGTAAGGGAACCCAAGTGACGGTGTCTTCT | |
| hIL10Ra | CAGGTACAACTCCAGGAGTCTGGCGGTGGGTCCGTGCAGGCAGGTGGCA | 219 |
| VHH15 | GCCTTCGCCTCTCCTGCGGGGCCTCCGGGTACACCTATAGTGGCTACTG | |
| | CATGGGGTGGTTCAGGCAAGCCCCCGGTAAGGAACGTGAGGGAGTTGCT | |
| | GTGATTGATTCAGATGGGTCCACGAGTTACGCTGACTCCGTGAAAGGTA | |
| | GGTTCACAATCTCCAAAGATAATGGCAAGAACACCCTCTACCTTCAGAT | |
| | GAATAGCCTGAAGCCAGAAGACACCGCCATGTATTACTGTGCTGCCGAC | |
| | CTGGGACACTATCGCCCTCCGTGCGGGGTCCTGTACTTGGGCATGGACT | |
| | ATTGGGGCAAGGGGACCCAGGTGACTGTGTCCTCT | |
| hIL10Ra | CAGGTGCAGTTGCAGGAATCCGGTGGAGGCTCTGTTCAGGCCGGGGGCT | 220 |
| VHH16 | CTCTCCGCCTGGCCTGCGCAGCCTCCAGGTATACTTACAGCAACTACTG | |
| | CATGGGGTGGTTTCGCCAGGCTCCGGGCAAAGAGCGTGAGGGAGTGGCT | |
| | ACTATTGATTCCGATGGAAACACCAGCTACGCCGATAGCGTGAAGGGCA | |
| | GATTTACTATCAGCAGAGATAACGCTAAAAACACGTTGTACCTCCAGAT | |
| | GAACTCACTCAAGCCGGGGGACACAGCTATGTATTACTGCGCAGCCGAT | |
| | CTGGGTCACTACCGCCCGCCCTGCGGCGCATATTACTATGGCATGGACT | |
| | ACTGGGGCAAGGGCACCCAGGTGACCGTGTCCAGT | |
| hIL10Ra | CAGGTGCAGCTCCAAGAGTCTGGCGGGGGTTCCGTGCAAGCCGGTGGCT | 221 |
| VHH17 | CACTCAGGTTGAGTTGCGCAGCCAGCGGCTATAGCAACTGTTCCTATGA | |
| | CATGACTTGGTATCGCCAGGCCCCTGGCAAAGAGCGTGAGTTCGTGTCA | |
| | GCTATTCACTCCGACGGCTCCACTCGTTATGCGGACTCTGTGAAGGGCC | |
| | GGTTTTTCATCTCCCAGGACAACGCTAAAAACACTGTCTATTTGCAGAT | |
| | GAACTCTCTGAAACCCGAAGATACCGCCATGTACTATTGCAAAACCGAT | |
| | CCTCTGCATTGTCGCGCCCACGGCGGGAGTTGGTACTCTGTGCGGGCCA | |
| | ACTATTGGGGCCAGGGCACCCAGGTCACCGTGTCCTCA | |
| hIL10Ra | CAGGTACAACTCCAGGAGTCTGGCGGTGGCAGCGTGCAGGCAGGCGGAA | 222 |
| VHH18 | GCCTGAGGCTGTCCTGCGCTGTATCTGGCTACACTTATAATTCCAACTG | |
| | CATGGGTTGGTTTCGGCAGGCTCCAGGTAAGGAGCGCGAGGGCGTCGCC | |
| | ACCATTTATACAGGTGTTGGCAGCACATATTACGCCGACAGCGTGAAGG | |
| | GAAGGTTCACCATCTCCCAAGACAATGCGAAAAACACAGTGTATCTCCA | |
| | GATGAATAGCCTGAAGCCCGAGGACACGGCTATGTATTACTGCGCTGCC | |
| | GAGCCACTGAGCAGAGTGTATGGGGGCAGCTGTCCTACACCCACTTTCG | |
| | GCTATTGGGGTCAAGGCACCCAGGTTACAGTCAGCTCC | |

TABLE 9
Nucleic Acid Sequences Encoding hIL10Rb VHHS

| Name | | SEQ |
|---|---|---|
| of VHH | DNA Sequence | ID NO. |
| IL10Rb | CAGGTGCAGCTTCAGGAATCAGGCGGAGGCAGCGTGCAGGCAGGGGGTA | 223 |
| VHH-1 | GCCTGCGTCTGTCTTGCGCAGCCAGCGGGTACACCTACAGCTCTGGCTG | |
| | TATGGGCTGGTTTCGCCAAGCCCCAGGAAAAGAACGGGAAGCCGTGGCG | |
| | GCTATCAATAGCGACGGCTCCACCTCCTATGCTGACTCCGTCAAAGGAC | |
| | GCTTCACCATTAGTAAAGATAACGCCAAGAACACCTTGTACCTTCAGAT | |
| | GAACTCCTTGAAACCGGAGGACACCGCAATGTATTACTGTGCGGCTGAG | |
| | CCCTACTGCTCAGGAGGCTACCCACGGTGGTCAGTGGCCGAGTTTGGTT | |
| | ATTGGGGCCAGGGCACCCAAGTGACTGTGTCCTCC | |
| hIL10Rb | CAGGTGCAACTCCAGGAGTCAGGGGGAGGTTCCGTGCAGGCTGGCGGTT | 224 |
| VHH-2 | CTCTCAGGTTGTCTTGCGCGGCCAGCGGCTATACGTACAGTAGCTACTG | |
| | CATGGGCTGGTTCCGGCAAGCCCCCGGCAAGGAGCGCGAAGGCGTGGCT | |
| | GCCATTGATTCCGATGGATCTACTAGGTATGCTGATAGTGTAAAGGGCC | |
| | GCTTCACAATCTCCAAGGACAATGCCAAGAACACACTGTATTTGCAAAT | |
| | GAACTCCCTCAAGCCCGAGGATACCGCTATGTACTATTGCGCTGCCGAA | |
| | CCATACTGTTCCGGTGGCTATAAGCGCACTATGGTGGCCGAGTTCGGAT | |
| | ACTGGGGTCAAGGCACACAGGTCACAGTGTCCTCT | |
| hIL10Rb | CAGGTGCAGTTGCAGGAGTCCGGGGGCGGTAGCGTTCAGGCTGGAGGGT | 225 |
| VHH-3 | CCCTGCGTCTGAGTTGTGCGGCATCTCGGTATACTTATAACAGTTACTG | |
| | TATGGGTTGGTTCCGCCAGGCACCTGGAAAGGAGCGGGAGGGGGTGGCG | |
| | ACTATTGATAGCGACGGAATGACCAGATATGCCGACTCTGTGAAGGGAA | |
| | GATTTACTATCTCAAAAGATAATGCCAAGAACACACTCTATTTGCAGAT | |
| | GAACAGCCTCAAGCCAGAGGATACCGCTATGTATTACTGTGCTGCCGAC | |
| | GCTGATTGCACCATCGCTGCCATGACGACCAACCCCTTGGGCCAGGGAA | |
| | CCCAAGTAACCGTCTCTAGC | |
| hIL10Rb | CAGGTCCAGCTCCAGGAATCTGGTGGCGGGTCTATCCAGGCGGGTGGCA | 226 |
| VHH-4 | GCCTGCGGCTGAGTTGCGCCGCTTCCCGCTACCTGTATAGTATTGATTA | |
| | TATGGCCTGGTTCAGGCAGTCACCGGGCAAAGAGCGCGAACCCGTCGCT | |
| | GTGATTTACACAGCCTCTGGTGCCACCTTCTATCCCGATAGTGTGAAGG | |
| | GCCGGTTCACTATCTCTCAAGACAACGCGAAGATGACTGTCTATCTTCA | |
| | GATGAACTCTCTGAAGTCCGAGGACACTGCCATGTATTACTGTGCCGCT | |
| | GTGCGCAAGACGGACTCTTATCTGTTCGATGCCCAGAGTTTCACTTACT | |
| | GGGGTCAGGGTACTCAGGTGACCGTATCCTCC | |
| hIL10Rb | CAGGTGCAGCTCCAGGAGTCTGGTGGCGGGCTGGTTCAGCCTGGGGGTT | 227 |
| VHH-5 | CACTCCGGTTGTCCTGCGCTGCGTCTGGTTATACCTACTCCAGCTACTG | |
| | TATGGGTTGGTTCCGCCAGGCACCGGGGAAGGAGAGGGAGGGCGTGGCT | |
| | CACATTGATTCTGATGGCTCTACGACCTACGCTGATAGCGTTAAGGGGC | |
| | GCTTCACTATCTCCAAGGATAACGCCAAGAACACCCTGTATCTGCAAAT | |
| | GAACAGCCTGAAGCCAGAAGACACTGCCATGTACTATTGCGCTGCCGAT | |
| | CCTATTCCCGGTCCTGGCTATTGTGACGGCGGTCCTAACAAGTACTGGG | |
| | GCCAAGGCACACAGGTGACTGTCAGTTCC | |
| hIL10Rb | CAGGTTCAACTCCAGGAATCCGGCGGTGGAAGCATTCAGGCGGGCGGTT | 228 |
| VHH-6 | CTTTGACTCTGAGCTGTGCGGCATCTCGGGACCTTTACAGCACTAACTA | |
| | TGTTGCCTGGTTCCGGCAGTCCCCCGGCAAGGAACGCGAAGCTGTGGCC | |
| | GTGATTTATACAGCCAGCGGCGCAACCCTGTATAGCGATTCAGTCAAAG | |
| | GCCGGTTCACCATCTCCCAGGACAACGCGAAGATGACCGTGTACCTGCA | |
| | AATGAACAGCCTGAAGTCTGAGGACACTGCCATGTATTACTGCGCAGCT | |
| | GTGAGAAAGACCGGACATTACCTCTTCGACGCCCAATCTTTCACCTACT | |
| | GGGGCCAGGGAACCCAGGTCACCGTCTCCTCT | |
| hIL10Rb | CAGGTGCAACTCCAGGAGTCAGGCGGTGGGTCCGTCCAGGCCGGTGGCT | 229 |
| VHH-7 | CCCTGAGGCTGAGTTGCGCCGCTTCCGGCTATACTTACTCCAGCGGTTG | |
| | CATGGGGTGGTTCCGCCAAGCCCCCGGTAAAGAACGCGAGGGAGTGGCT | |
| | ACAATTAACTCCGATGGAAGCACTAACTACGCCGACTCTGTGAAGGGAC | |
| | GCTTCACCATTAGCAAAGACAATGCTAAGAACACCCTTTACCTTCAAAT | |
| | GAACAGCCTGAAGCCTGAGGATACCGCTATGTATTACTGTGCCGCAGAA | |
| | CCGTATTGTAGCGGTGGCTACCCTCGCTGGTCCGTCGCCGAGTTCGGTT | |
| | ATTGGGGCCAGGGGACCCAAGTGACTGTTTCTAGC | |
| hIL10Rb | CAGGTGCAACTTCAGGAGAGCGGCGGGGGCTCTGTGCAAGCTGGTGGCT | 230 |
| VHH-8 | CCCTGCGGCTCAGCTGTGCTGCCTCTGGGTATTCTTACAGTAGCTACTG | |
| | TATGGGCTGGTTCAGACAGGCACCAGGCAAGGAGCGCGAGGGTGTGGCG | |
| | GCCATCGCTTCCGACGGGAGTACCAGCTACGCCGACAGCGTTAAAGGTA | |
| | GGTTTGCCATCTCTAAGGATAATGCGAAGAATACACTCTACCTTCAGAT | |
| | GGCTAGTCTGAAGCCAGAGGATACCGCCATGTATTACTGCGCGGCAGAG | |
| | CCCTGGTGCACGGGAGGGTATTCACGCCTGACCCCGGCTGAGTATGGAT | |
| | ACTGGGGGCAGGGCACCCAGGTGACCGTTAGCTCC | |
| hIL10Rb | CAGGTCCAGTTGCAGGAAAGCGGAGGGGGCCTGGTGCAGCCAGGAGGTT | 231 |
| VHH-9 | CTCTGAGACTGAGCTGTGCCGCTTCTGGTTACACATATTCTAGCGGGTG | |
| | CATGGGCTGGTTCCGCCAGGCTCCCGGCAAGGAACGTGAGGGTGTGGCA | |
| | ACTATCAATTCCGACGGCTCTACAAACTACGCAGATTCTGTTAAAGGCC | |
| | GCTTCACAATCTCTAAGGACAACGCCAAAAACACTCTGTACTTGCAGAT | |
| | GAATAGCCTGAAGCCTGAAGACACTGCCATGTACTATTGCGCAGCTGAG | |
| | CCCTACTGTTCTGGAGGCTACCCCCGCTGGTCTGTGGCCGAGTTCGGTT | |
| | ACTGGGGACAAGGAACCCAGGTCACAGTGTCCAGT | |
| hIL10Rb | CAGGTTCAGCTCCAGGAGTCAGGCGGGGGTCTTGTCCAGCCTGGTGGCT | 232 |
| VHH-10 | CCCTGCGCCTGTCCTGTGCTGCCTCCGGTTACACCTACTCCAGCTATTG | |
| | CATGGGATGGTTCAGACAAGCGCCAGGCAAGGAACGTGAGGGGGTCGCC | |
| | CACATTGACTCCGACGGTTCCACTACCTACGCCGACAGCGTCAAAGGCC | |
| | GCTTCGCGATTTCTAAGGATAACGCTAAGAATACTCTGTACTTGCAGAT | |
| | GAACTCACTGAAGCCAGAGGACACGGCCATGTATTACTGCGCAGCCGAT | |
| | CCGATCCCCGGCCCCGGCTATTGTGACGGTGGCCCGAACAAGTACTGGG | |
| | GACAGGGCACCCAAGTGACGGTGTCCTCT | |
| hIL10Rb | CAGGTACAGTTGCAGGAGAGCGGAGGCGGTTCCGTGCAGGCAGGTGGCT | 233 |
| VHH-11 | CTCTTAGACTGTCCTGCGCCGCGAGCGGGTACACCTACAGTAGCTATTG | |
| | TATGGGCTGGTTCCGCCAGGCTCCTGGTAAGGGTCGCGAGGGCGTCGCT | |
| | GCCATCGACTCCGATGGCTCTACTCGCTACGCAGATTCTGTCAAGGGGC | |
| | GCTTCACAATTTCCAAGGACAACGCCAAGAACACGCTTTACTTGCAGAT | |
| | GAACTCACTGAAGCCGGAGGACACCGCTATGTATTACTGCGCTGCCGAG | |
| | CCCTACTGTTCTGGGGGCTACAAGCGCACTATGGTGGCCGAGTTCGGAT | |
| | ATTGGGGCCAGGGTACACAGGTGACCGTCAGTTCT | |
| hIL10Rb | CAGGTGCAGTTGCAGGAGTCTGGCGGTGGCTCTGTGCAGGCTGGGGGCT | 234 |
| VHH-12 | CTCTGCGCCTGAGTTGCGCTGCCAGCGGTTACACCTACTCCAGCTATTG | |
| | TATGGGATGGTTCCGCCAGGCTCCGGGGAAGGAGAGGGAGGGCGTGGCC | |
| | CATATCGACTCTGATGGCTCCACATCCTACGCCGACAGCGTGAAGGGAC | |
| | GTTTCACCATTAGCAAGGACAATGCGAAGAATACCCTCTACTTGCAGAT | |
| | GAACTCCCTGAAGCCGGAGGATACTGCCATGTATTACTGCGCCGCTGAT | |
| | CCCATCCCAGGGCCTGGGTACTGTGACGGAGGCCCGAACAAGTATTGGG | |
| | GACAAGGAACGCAGGTCACAGTGTCATCT | |
| hIL10Rb | CAGGTACAACTCCAGGAGAGTGGTGGAGGCTCCGTTCAAGCCGGGGGCT | 235 |
| VHH-13 | CCCTGCGGCTGTCCTGTGCGGCCAGCGGTTACACCTATTCATCTTACTG | |
| | TATGGGCTGGTTCCGGCAGGCCCCTGGTAAGGAAAGAGAGGGTGTCGCT | |
| | CACATTGATTCCGACGGTAGTACCTCTTACGCAGACTCTGTCAAGGGCA | |
| | GGTTCACCATCTCTAAGGACAATGCCAAGAACACCTTGTACCTCCAGAT | |
| | GAACTCTCTGAAGCCCGAGGACACTGCAATGTACTATTGTGCGGCTGAC | |
| | CCTATTCCCGGCCCTGGATATTGCGACGGCGGACCTAACAATTACTGGG | |
| | GACAGGGCACCCAGGTCACCGTCAGCTCC | |
| hIL10Rb | CAGGTTCAGCTCCAAGAATCCGGCGGGGGCTCTGTGCAGGCGGGCGGAA | 236 |
| VHH-14 | GTCTGCGTCTGTCATGCGCTGCCAGCGGGTACACTTACTCTTCCGGTTG | |
| | TATGGGCTGGTTTAGGCAGGCTCCGGGAAAGGAAAGGGAGGGCGTCGCA | |
| | ACTATCAACAGCGACGGCTCTACGAACTACGCTGACTCTGTGAAAGGCC | |
| | GCTTCACCATCAGCAAAGACAACGCCAAAAATACACTGTATCTCCAGAT | |
| | GAATAGCTTGAAACCCGAGGACACCGGAATGTATTACTGCGCGGCAGAG | |
| | CCATACTGTTCAGGCGGTTACCCAAGATGGTCCGTGGCTGAGTTCGGTT | |
| | ATTGGGGGCAGGGCACTCAGGTTACTGTGTCTTCC | |
| hIL10Rb | CAGGTGCAGCTCCAGGAATCCGGGGGCGGTTCTGTGCAGGCTGGTGGCT | 237 |
| VHH-15 | CTCTGCGCCTGTCTTGCACTGTTTCCAGGTACACTGCCTCTGTAAACTA | |
| | TATGGGCTGGTTTAGACAAGCTCCGGGCAAGGAACGCGAAGGCGTCGCT | |
| | ACCATCTTTACAGGTGCAGGTACGACCTATTACGCCAATAGCGTTAAAG | |
| | GGAGGTTCACCATCTCCAGGGACAATGCCAAAAACACAGCCTATCTCCA | |
| | GATGAACTCCCTCAAACCTGAAGACACAGCCATCTACTATTGCGCGGTT | |
| | GACTTCCGTGGTGGCCTGCTCTATAGACCGGCGTATGAGTACACCTACC | |
| | GTGGACAAGGCACCCAAGTCACAGTGAGCAGC | |
| hIL10Rb | CAGGTGCAGCTCCAAGAGTCCGGCGGAGGGAGTGTAGAGGCTGGCGGGT | 238 |
| VHH-16 | CCCTGCGCCTTAGCTGCGCGGCCAGCGGCTATACACACAGTTCTTATTG | |
| | TATGGGTTGGTTCCGCCAAGCTCCGGGAAAGGAGCGTGAGGGCGTGGCT | |
| | GCCATCGACGTGGATGGCTCCACAACCTACGCCGACAGCGTGAAGGGCA | |
| | GGTTTACGATCTCTAAGGATAACGCTAAGAATACTCTCTATTTGCAGAT | |
| | GAACTCCCTCAAACCCGAGGATACAGGAATGTACTATTGCGCTGCCGAG | |
| | TTCGCCGACTGCTCAAGCAATTATTTCCTGCCTCCAGGAGCCGTTAGGT | |
| | ACTGGGGCCAGGGGACTCAGGTCACAGTAAGCAGC | |
| hIL10Rb | CAGGTGCAGCTCCAGGAGAGCGGTGGCGGATCAGTGCAGGCTGGAGGCT | 239 |
| VHH-17 | CCCTCAGACTGTCCTGCACCGTGAGCCGCTATACCGCCTCCGTCAACTA | |
| | TATGGGATGGTTTAGGCAGGCTCCGGGCAAGGAGCGCGAGGGGGTCGCG | |
| | ACTATCTTCACCGGAGCCGGTACTACCTATTACGCTAATTCTGTTAAAG | |
| | GCCGCTTTACCATTAGTCGCGACAACGCTAAGAACACAGCTTACCTCCA | |
| | GATGAACTCTCTGAAGCCAGAGGATACCGCCATGTATTACTGCGCCGTG | |
| | GACTTCCGGGGCGGTTTGCTCTACCGCCCGGCCTACGAATACACCTATC | |
| | GCGGCCAGGGCACGCAGGTCACGGTGTCCTCA | |
| hIL10Rb | CAGGTGCAGCTCCAAGAGTCCGGTGGAGGCAGCGTCCAGGCCGGGGGTA | 240 |
| VHH-18 | GTCTTAGGCTCAGCTGTGCTGCCAGTGGAGACACCTACTCTTCCTATTG | |
| | CATGGGATGGTTCAGACAGGCCCCCGGCAAAGAGCGCGAGGGCGTTGCA | |
| | TTCATCGACTCCGACGGCTCCACTCGCTACGCCGATAGCGTGGAGGGCC | |
| | GTTTTACCATCTCCAAGGACAACGCGAAGAACACTCTGTATCTGCAAAT | |
| | GAACTCCCTGAAGCCCGAAGACACCGCCATGTACTATTGCGCGGCTGAG | |
| | CCATACTGTAGTGGCGGATATCATCGTAAGGAAATGGCAGAGTTCGGCT | |
| | ATTGGGGCCAGGGCACCCAGGTCACTGTGAGTTCC | |
| hIL10Rb | CAGGTGCAGTTGCAGGAATCCGGCGGAGGCTCTGTGCAGGCGGGCGGTT | 241 |
| VHH-19 | CCCTCCGCCTGAGTTGTGCCGCGTCTGGCTATACTTACTCTTCCTATTG | |
| | TATGGGATGGTTCCGGCAAGCGCCCGGCAAAGAGCGGGAGGGCGTTGCG | |
| | CATATCGACAGTGATGGTAGCACCAGTTACGCTGATAGCGTGAAAGGCA | |
| | GATTCACTATCTCAAAGGATAACGCGAAGAACACTCTTTACCTCCAGAT | |
| | GAACTCCCTTAAACCTGAGGATACCGCGATGTATTACTGTGCTGCCGAC | |
| | CCCATTCCCGGCCCTGGATACTGTGACGGAGGCCCTAACAAGTACCGTG | |
| | GGCAAGGAACACAGGTCACAGTGTCCAGC | |
| hIL10Rb | CAGGTGCAACTCCAGGAGTCTGGCGGGGGCAGCGTCCAGGCAGGTGGAA | 242 |
| VHH-20 | GTCTCCGTCTCTCATGTGCTGCCAGCGGCTATACATACTCCAGCTACTG | |
| | TATGGGATGGTTTAGACAGGCACCCGGCAAGGAGCGCGAAGGGGTGGCC | |
| | CATATCGACTCCGATGGCAGCACAACCTATGCCGACTCTGTGAAAGGGC | |
| | GGTTCGCCATCTCCAAGGACAACGCTAAGAATACCCTGTACCTCCAGAT | |
| | GAACTCTCTGAAGCCTGAGGACACCGCCATGTATTACTGCGCTGCCGAC | |
| | CCAATCCCTGGCCCAGGTTACTGCGATGGGGGACCAAACAAATATTGGG | |
| | GACAGGGCACGCAGGTTACAGTCTCCAGC | |
| hIL10Rb | CAGGTCCAACTCCAGGAAAGTGGAGGTGGCTCTGTTCAGGCCGGGGGCA | 243 |
| VHH-21 | GCCTGAGGCTGAGCTGCACCGGCTCAGGCTATACAGCCAGTAATAACTG | |
| | CATGGGCTGGTTCCGTCAAGCGCCCGGCAAAGAGCGTGAAGGTGTGGCC | |
| | GTAATTTTTACCGGCGCTGGCACCAGCTATTACGACAGTTCCGTGGGCC | |
| | GTCTGTTCATCAGCTCACAGGACGCCGCTTCCACCCTCGATCAGTTGCT | |
| | GATGAGCCTTCTGCCCGATGACACCGCAGTAATGTACTGTGGAGCCGAA | |
| | GATGACTGCACACTGCTCCTGATGACGCCAAACCCCGATGACCAATGGT | |
| | CCCGCCTGAGTGTGTCCTCC | |
| hIL10Rb | CAGGTGCAGCTCCAGGAGAGCGGGGGCGGTTCTGTTCAGGCGGGAGGCA | 244 |
| VHH-22 | GCCTGCGTCTGTCCTGTGCAGCCTCTGGTTACACAGACAGTCGTTACTG | |
| | CATGGGCTGGTTCCGCAAGGCACCTGGAAAGGAGCGCGAGGGTGTTGCG | |
| | CACATCGACTCCGACGGGAGCACTAGCTATGCTGACAGCGTGAAGGGGC | |
| | GCTTCACTATCAGCAAGGATAACGCGAAAAACACCTTGTACCTTCAGAT | |
| | GAACTCCCTCAAACCCGAAGACACAGCGATGTACTATTGTGCCGCTGAT | |
| | CCGATCCCAGGGCCTGGCTACTGTGATGGTGGACCTAATAAGTACTGGG | |
| | GGCAGGGAACTCAGGTGACCGTGTCATCA | |
| hIL10Rb | CAGGTCCAGTTGCAGGAATCTGGAGGCGGTTCCGTGCAAGCAGGGGGCT | 245 |
| VHH-23 | CACTCAGACTGTCCTGCGCTGCCAGCGGCTACACTTACTCTTCATATTG | |
| | CATGGGCTGGTTCCGCCAGGCACCGGGCAAGGAGCGGGAAGGCGTGGCC | |
| | GCTATTGATAGCGATGGCTCTACGCGCTACGCAGATAGCGTGAAAGGGA | |
| | GGTTCACGATCTCCAAAGATAATGCCAAGAAAATTCTGTATCTCCAGAT | |
| | GAACTCTCTGAAGGTCGAGGACACCGCCATGTACTATTGTGCAGCCGAA | |
| | CCCTACTGTTCTGGTGGCTACAAGAGGACTATGGTGGCCGAGTTCGGCT | |
| | TCTGGGGCCAGGGGACCCAAGTGACTGTCAGTAGC | |
| hIL10Rb | CAGGTGCAACTTCAGGAGAGCGGTGGCGGATCTGTGCAGGCTGGAGGGT | 246 |
| VHH-24 | CTCTGAAGCTGTCCTGCGCGGCCAGCGGTTACACATACAGTAGCTACTG | |
| | CATGGGATGGTTTCGTCAGGCCCCAGGCAAGGAGCGCGAAGGAGTGGCG | |
| | CACATCGACTCCGATGGGTCCACCACATACGCCGACTCCGTGAAGGGCC | |
| | GTTTCACAATCAGCAAGGATAACGCGAAGAACACGCTGTACTTGCAGAT | |
| | GAACTCTCTCAAACCAGAGGACACTGCAATGTACTATTGCGCGGCTGAC | |
| | CCCATCCCTGGCCCTGGTTACTGTGACGGTGGCCCCAACAATTACTGGG | |
| | GGCAAGGGACCCAAGTCACCGTGTCCTCC | |
| hIL10Rb | CAGGTCCAGCTCCAGGAGTCCGGCGGGGGCTCCGTCCAGGCAGGGGGCT | 247 |
| VHH-25 | CCCTGCGTCTGTCATGCGCCGCTTCTGGGTATACCTACAGTTCCTATTG | |
| | TATGGGTTGGTTTCGCCAAGCACCCGGTAAGGAGCGCGAAGGTATTGCG | |
| | CACATTGATAGCGATGGCTCCACAACCTATGCTGACAGTGTGAAAGGAC | |
| | GCTTCACTATTTCCAAGGATAACGCTAAGAACACACTCTACCTTCAGAT | |
| | GAACAGCCTGAAGCCGGAAGACACCGCAATGTACTATTGTGCAGCTGAC | |
| | CCCATTCCTGGACCCGGTTACTGTGATGGAGGTCCTAATAACTATTGGG | |
| | GACAGGGCACTCAAGTGACCGTCTCAAGC | |
| hIL10Rb | CAGGTGCAGTTGCAGGAGAGCGGGGGTGGCTCTGTGCAGGCCGGGGGCT | 248 |
| VHH-26 | CCCTGAGGCTGAGCTGCGCGGCCAGCGGGTACAGCTACTCTAGCTATTG | |
| | CATGGGTTGGTTCCGCCAGGCCCCTGGCAAGGAGCGCGAGGGAGTGGCC | |
| | ACGATTGACTCAGATGGCATGACCCGTTATGCGGATTCCGTCAAGGGGC | |
| | GCTTCACCATCAGCAAAGATAACGCCAAAAATACCCTGTACTTGCAGAT | |
| | GAACTCACTGAAACCTGAGGATACAGCCATGTATTACTGCGCAGCTCCG | |
| | CTCTATGACTGTGACTCTGGTGCCGTGGGTAGAAACCCACCTTACTGGG | |
| | GGCAGGGAACCCAGGTGACCGTGTCCTCA | |
| hIL10Rb | CAGGTCCAGCTCCAGGAAAGCGGTGGGGGCAGCGTCCAAACAGGGGGTA | 249 |
| VHH-27 | GCCTGCGCCTCTCTTGCGCAGCCAGCGGCTACACATATCTGCGCGGATG | |
| | TATGGGCTGGTTCCGCCAGGCCCCTGGTAAGGAAAGAGAGGGGGTGGCC | |
| | GTGATGGACGTGGTTGGAGACAGACGTTCCTACATTGATTCCGTGAAGG | |
| | GCCGCTTTACTATCTCACGCGATAACGCGGCTAACTCTGTGTATTTGCA | |
| | GATGGATAACCTGAAGCCCGAGGACACCGCTATGTACTATTGCACAGCT | |
| | GGTCCCAACTGTGTCGGTTGGCGCTCCGGCCTGGACTATTGGGGTCAGG | |
| | GAACCCAGGTTACAGTTAGCAGT | |

TABLE 10
Nucleic Acid Sequences Encoding mIL10Rb VHHS

| | | SEQ |
|---|---|---|
| | | ID |
| Name | DNA Sequence | NO |
| DR1322 | CAGGTGCAGCTCCAGGAGAGTGGTGGCGGTTCTGTCCAAGCTGGCG | 250 |
| DNA | GAGCCCTGCGCCTGTCCTGCACAGCAAGCGGCTACACCGCCTCTAG | |
| | CATTTGCATGGGATGGTTCCGTCAGGCCCCAGGCAAGGAGAGGGAG | |
| | AGAGTGGCTGTGATTACCACGGCAGCCTCCGGTACTTACTATGCCG | |
| | ACTCTGTGAATGGCCGCTTCTCAATCTCTCAGAATAACGCCAAAAA | |
| | TACTGTGTACCTCCAGATGAACTCCCTGAAACCTGACGATACCGCG | |
| | ATGTATTACTGCGCAGCCACCCGGCGCGGCGGTGACTGCCTGGACC | |
| | CATTGCAGACCCCAGCCTATAATACCTGGGGCCAGGGAACCCAGGT | |
| | CACCGTCTCTTCT | |
| DR1323 | CAGGTGCAGCTCCAGGAAAGCGGCGGTGGCTCCGTCCAGGCCGGTG | 251 |
| DNA | GCTCCCTGAGGCTGAGCTGTGTGGCTTCCGGCGATACTTATTCTCG | |
| | CAAGTACATCGCATGGGTGCGTCAGGTGCCCGGTAAAGAACGTGAG | |
| | GGAGTGGCAGTGATGTATACCCCAGGCTCCGCTACTTACTATACAG | |
| | ACACAGTGATGGGTCGTTTCACCATCTCCCAGGACAACGCCAAGAA | |
| | CACTGTGTACCTTCAAATGAACAGCCTCAAACCTGAAGACACCGCC | |
| | ATGTACTTTTGCGCGGCCAAGGCCAGCGGCTCCATGTTTAACTTCC | |
| | GCGATTACACTTATTGGGGACAGGGCACTCAGGTGACCGTAAGCTC | |
| | T | |
| DR1324 | CAGGTGCAGCTGCAAGAAAGCGGAGGTGGCTCTGTCCAGGCAGGAG | 252 |
| DNA | GCTCCCTCCGGCTTAGCTGCGCTACCAGCGGGTATGCTTCCTGTTC | |
| | CCGCGCCATGAGGTGGTACAGGCAGGCACCGGGCAAGGAGCGCGAA | |
| | TTTGTGGCGTACATCGACGGGGTGGGCAGTACTGGTTATGCGGACA | |
| | GCGTTAAAGGCCGGTTTACCATCTCCCAAGATAATGCAAAGTACAC | |
| | GGCTTACTTGCAGATGAACTCCCTCAAGCCTGAGGATACCGCGATG | |
| | TATTACTGTAATCGGGGCTGTAGAGCCGATGGTAGCAATAGTCTGG | |
| | ACAACTACTGGGGCCAGGGCACACAGGTGACTGTCTCTTCA | |
| DR1325 | CAGGTGCAGTTGCAGGAGTCCGGCGGTGGCAGCGTTCAGGCGGGCG | 253 |
| DNA | GTAGCCTGCGTCTGAGCTGCGCCGCGTCCGGCTACACCTATAACCG | |
| | TCGCTTCATGGGTTGGTTCCGTCAAGCGCCCGGCAAGGAGAGAGAG | |
| | GGCCTCGCCATTATCTACACCCCCAACAGCTCCACCTTCTACGCCG | |
| | ACTCTGTGACGGGCCGCTTTACAATCTCACAGGATTCTGCCCGCAA | |
| | CACCGTCTATTTGCAGATGAACTCCCTGAAACCTGAGGACACCGCT | |
| | ATGTACTATTGTGCAGCCGCTCGCATCGCTTCTATGACTGAGCTTT | |
| | CAGTGAGAGATATGGACTATTGGGGCAAGGGCACCCAGGTGACCGT | |
| | TTCCTCC | |
| DR1326 | CAGGTACAACTCCAGGAGAGCGGGGGAGGTAGCGTACAGGCTGGCG | 254 |
| DNA | GGTCCTTGCGTCTGAGCTGCACTGCATCTCGTTACATCGCTCTTAA | |
| | TGCGTGTATGGCTTGGATTCGGCAGGCCCCCGGCTCCGAAAGGGAG | |
| | GTCGTGGCCACAATCGTGACTGATGGCTCCAGAACCTATTACGCAG | |
| | ACTCTGTCAAGGGCCGGTTTACTATCTCTCAAGACAACGCCAAGAA | |
| | CACCATGTACCTCCAGATGAACGGTTTGAAACCCGAAGACACCGCC | |
| | ATGTATTACTGTGCAGCCGACAGGCGCTGCCCCGTGTCCAGAGCCC | |
| | CATACGAATACGAACTGCGCTACTGGGGTCAGGGCACCCAGGTGAC | |
| | TGTCAGCAGC | |
| DR1327 | CAAGTCCAGCTTCAAGAAAGCGGAGGGGGCTCTGTTCAGGCAGGCG | 255 |
| DNA | GGTCCCTCCGGCTGTCCTGCGCTGCCTCCGGCTACACATACAACGG | |
| | AAAGTGCATGGCTTGGTTCCGCCAGGCTCCCGGCAAGGAGCGCGAA | |
| | GTCGTGGCTGGCATTTACACCGGGGGCTCCAGCACATATTACGCCG | |
| | ATAGTGTGAAGGGACGCTTTACGATTTCCCAAGACAATGCTAAAAA | |
| | TACAGTCTATCTCCAGATGGACAGCCTGAAGCCCGAAGACACTGCC | |
| | ATGTATTACTGCGCCACCAGCAGAAGCTGTAGCGACCTGCGCAGAC | |
| | GCTCCATCGCCTACTGGGGACAGGGGACTCAGGTCACCGTCAGCTC | |
| | T | |

TABLE 11
Description of IL10R Receptor Binding Proteins Evaluated

| IL10 | | | | |
|---|---|---|---|---|
| Binding | N-terminal | | C-terminal | |
| Protein | sdAb | | sdAb | C-terminal |
| (SEQ ID | (SEQ ID | | (SEQ ID | purification |
| NO: ) | NO:) | Linker | NO:) | handle |
| 256 | 163 | G3S (SEQ ID | 172 | ASH6 (SEQ ID |
| | | NO: 484) | | NO: 560) |
| 257 | 163 | G3S (SEQ ID | 183 | ASH6 (SEQ ID |
| | | NO: 484) | | NO: 560) |
| 258 | 163 | G3S (SEQ ID | 174 | ASH6 (SEQ ID |
| | | NO: 484) | | NO: 560) |
| 259 | 163 | G3S (SEQ ID | 186 | ASH6 (SEQ ID |
| | | NO: 484) | | NO: 560) |
| 260 | 163 | G3S (SEQ ID | 187 | ASH6 (SEQ ID |
| | | NO: 484) | | NO: 560) |
| 261 | 163 | G3S (SEQ ID | 197 | ASH6 (SEQ ID |
| | | NO: 484) | | NO: 560) |
| 262 | 163 | G3S (SEQ ID | 198 | ASH6 (SEQ ID |
| | | NO: 484) | | NO: 560) |
| 263 | 159 | G3S (SEQ ID | 172 | ASH6 (SEQ ID |
| | | NO: 484) | | NO: 560) |
| 264 | 159 | G3S (SEQ ID | 183 | ASH6 (SEQ ID |
| | | NO: 484) | | NO: 560) |
| 265 | 159 | G3S (SEQ ID | 174 | ASH6 (SEQ ID |
| | | NO: 484) | | NO: 560) |
| 266 | 159 | G3S (SEQ ID | 186 | ASH6 (SEQ ID |
| | | NO: 484) | | NO: 560) |
| 267 | 159 | G3S (SEQ ID | 187 | ASH6 (SEQ ID |
| | | NO: 484) | | NO: 560) |
| 268 | 159 | G3S (SEQ ID | 197 | ASH6 (SEQ ID |
| | | NO: 484) | | NO: 560) |
| 269 | 159 | G3S (SEQ ID | 198 | ASH6 (SEQ ID |
| | | NO: 484) | | NO: 560) |
| 270 | 161 | G3S (SEQ ID | 172 | ASH6 (SEQ ID |
| | | NO: 484) | | NO: 560) |
| 271 | 161 | G3S (SEQ ID | 183 | ASH6 (SEQ ID |
| | | NO: 484) | | NO: 560) |
| 272 | 161 | G3S (SEQ ID | 174 | ASH6 (SEQ ID |
| | | NO: 484) | | NO: 560) |
| 273 | 161 | G3S (SEQ ID | 186 | ASH6 (SEQ ID |
| | | NO: 484) | | NO: 560) |
| 274 | 161 | G3S (SEQ ID | 187 | ASH6 (SEQ ID |
| | | NO: 484) | | NO: 560) |
| 275 | 161 | G3S (SEQ ID | 197 | ASH6 (SEQ ID |
| | | NO: 484) | | NO: 560) |
| 276 | 161 | G3S (SEQ ID | 198 | ASH6 (SEQ ID |
| | | NO: 484) | | NO: 560) |
| 277 | 162 | G3S (SEQ ID | 172 | ASH6 (SEQ ID |
| | | NO: 484) | | NO: 560) |
| 278 | 162 | G3S (SEQ ID | 183 | ASH6 (SEQ ID |
| | | NO: 484) | | NO: 560) |
| 279 | 162 | G3S (SEQ ID | 174 | ASH6 (SEQ ID |
| | | NO: 484) | | NO: 560) |
| 280 | 162 | G3S (SEQ ID | 186 | ASH6 (SEQ ID |
| | | NO: 484) | | NO: 560) |
| 281 | 162 | G3S (SEQ ID | 187 | ASH6 (SEQ ID |
| | | NO: 484) | | NO: 560) |
| 282 | 162 | G3S (SEQ ID | 197 | ASH6 (SEQ ID |
| | | NO: 484) | | NO: 560) |
| 283 | 162 | G3S (SEQ ID | 198 | ASH6 (SEQ ID |
| | | NO: 484) | | NO: 560) |
| 284 | 165 | G3S (SEQ ID | 172 | ASH6 (SEQ ID |
| | | NO: 484) | | NO: 560) |
| 285 | 165 | G3S (SEQ ID | 183 | ASH6 (SEQ ID |
| | | NO: 484) | | NO: 560) |
| 286 | 165 | G3S (SEQ ID | 174 | ASH6 (SEQ ID |
| | | NO: 484) | | NO: 560) |
| 287 | 165 | G3S (SEQ ID | 186 | ASH6 (SEQ ID |
| | | NO: 484) | | NO: 560) |
| 288 | 165 | G3S (SEQ ID | 187 | ASH6 (SEQ ID |
| | | NO: 484) | | NO: 560) |
| 289 | 165 | G3S (SEQ ID | 197 | ASH6 (SEQ ID |
| | | NO: 484) | | NO: 560) |
| 290 | 165 | G3S (SEQ ID | 198 | ASH6 (SEQ ID |
| | | NO: 484) | | NO: 560) |
| 291 | 167 | G3S (SEQ ID | 172 | ASH6 (SEQ ID |
| | | NO: 484) | | NO: 560) |
| 292 | 167 | G3S (SEQ ID | 183 | ASH6 (SEQ ID |
| | | NO: 484) | | NO: 560) |
| 293 | 167 | G3S (SEQ ID | 174 | ASH6 (SEQ ID |
| | | NO: 484) | | NO: 560) |
| 294 | 167 | G3S (SEQ ID | 186 | ASH6 (SEQ ID |
| | | NO: 484) | | NO: 560) |
| 295 | 167 | G3S (SEQ ID | 187 | ASH6 (SEQ ID |
| | | NO: 484) | | NO: 560) |
| 296 | 167 | G3S (SEQ ID | 197 | ASH6 (SEQ ID |
| | | NO: 484) | | NO: 560) |
| 297 | 167 | G3S (SEQ ID | 198 | ASH6 (SEQ ID |
| | | NO: 484) | | NO: 560) |
| 298 | 170 | G3S (SEQ ID | 172 | ASH6 (SEQ ID |
| | | NO: 484) | | NO: 560) |
| 299 | 170 | G3S (SEQ ID | 183 | ASH6 (SEQ ID |
| | | NO: 484) | | NO: 560) |
| 300 | 170 | G3S (SEQ ID | 174 | ASH6 (SEQ ID |
| | | NO: 484) | | NO: 560) |
| 301 | 170 | G3S (SEQ ID | 186 | ASH6 (SEQ ID |
| | | NO: 484) | | NO: 560) |
| 302 | 170 | G3S (SEQ ID | 187 | ASH6 (SEQ ID |
| | | NO: 484) | | NO: 560) |
| 303 | 170 | G3S (SEQ ID | 197 | ASH6 (SEQ ID |
| | | NO: 484) | | NO: 560) |
| 304 | 170 | G3S (SEQ ID | 198 | ASH6 (SEQ ID |
| | | NO: 484) | | NO: 560) |
| 305 | 172 | G3S (SEQ ID | 163 | ASH6 (SEQ ID |
| | | NO: 484) | | NO: 560) |
| 306 | 172 | G3S (SEQ ID | 159 | ASH6 (SEQ ID |
| | | NO: 484) | | NO: 560) |
| 307 | 172 | G3S (SEQ ID | 161 | ASH6 (SEQ ID |
| | | NO: 484) | | NO: 560) |
| 308 | 172 | G3S (SEQ ID | 162 | ASH6 (SEQ ID |
| | | NO: 484) | | NO: 560) |
| 309 | 172 | G3S (SEQ ID | 165 | ASH6 (SEQ ID |
| | | NO: 484) | | NO: 560) |
| 310 | 172 | G3S (SEQ ID | 167 | ASH6 (SEQ ID |
| | | NO: 484) | | NO: 560) |
| 311 | 172 | G3S (SEQ ID | 170 | ASH6 (SEQ ID |
| | | NO: 484) | | NO: 560) |
| 312 | 183 | G3S (SEQ ID | 163 | ASH6 (SEQ ID |
| | | NO: 484) | | NO: 560) |
| 313 | 183 | G3S (SEQ ID | 159 | ASH6 (SEQ ID |
| | | NO: 484) | | NO: 560) |
| 314 | 183 | G3S (SEQ ID | 161 | ASH6 (SEQ ID |
| | | NO: 484) | | NO: 560) |
| 315 | 183 | G3S (SEQ ID | 162 | ASH6 (SEQ ID |
| | | NO: 484) | | NO: 560) |
| 316 | 183 | G3S (SEQ ID | 165 | ASH6 (SEQ ID |
| | | NO: 484) | | NO: 560) |
| 317 | 183 | G3S (SEQ ID | 167 | ASH6 (SEQ ID |
| | | NO: 484) | | NO: 560) |
| 318 | 183 | G3S (SEQ ID | 170 | ASH6 (SEQ ID |
| | | NO: 484) | | NO: 560) |
| 319 | 174 | G3S (SEQ ID | 163 | ASH6 (SEQ ID |
| | | NO: 484) | | NO: 560) |
| 320 | 174 | G3S (SEQ ID | 159 | ASH6 (SEQ ID |
| | | NO: 484) | | NO: 560) |
| 321 | 174 | G3S (SEQ ID | 161 | ASH6 (SEQ ID |
| | | NO: 484) | | NO: 560) |
| 322 | 174 | G3S (SEQ ID | 162 | ASH6 (SEQ ID |
| | | NO: 484) | | NO: 560) |
| 323 | 174 | G3S (SEQ ID | 165 | ASH6 (SEQ ID |
| | | NO: 484) | | NO: 560) |
| 324 | 174 | G3S (SEQ ID | 167 | ASH6 (SEQ ID |
| | | NO: 484) | | NO: 560) |
| 325 | 174 | G3S (SEQ ID | 170 | ASH6 (SEQ ID |
| | | NO: 484) | | NO: 560) |
| 326 | 186 | G3S (SEQ ID | 163 | ASH6 (SEQ ID |
| | | NO: 484) | | NO: 560) |
| 327 | 186 | G3S (SEQ ID | 159 | ASH6 (SEQ ID |
| | | NO: 484) | | NO: 560) |
| 328 | 186 | G3S (SEQ ID | 161 | ASH6 (SEQ ID |
| | | NO: 484) | | NO: 560) |
| 329 | 186 | G3S (SEQ ID | 162 | ASH6 (SEQ ID |
| | | NO: 484) | | NO: 560) |
| 330 | 186 | G3S (SEQ ID | 165 | ASH6 (SEQ ID |
| | | NO: 484) | | NO: 560) |
| 331 | 186 | G3S (SEQ ID | 167 | ASH6 (SEQ ID |
| | | NO: 484) | | NO: 560) |
| 332 | 186 | G3S (SEQ ID | 170 | ASH6 (SEQ ID |
| | | NO: 484) | | NO: 560) |
| 333 | 187 | G3S (SEQ ID | 163 | ASH6 (SEQ ID |
| | | NO: 484) | | NO: 560) |
| 334 | 187 | G3S (SEQ ID | 159 | ASH6 (SEQ ID |
| | | NO: 484) | | NO: 560) |
| 335 | 187 | G3S (SEQ ID | 161 | ASH6 (SEQ ID |
| | | NO: 484) | | NO: 560) |
| 336 | 187 | G3S (SEQ ID | 162 | ASH6 (SEQ ID |
| | | NO: 484) | | NO: 560) |
| 337 | 187 | G3S (SEQ ID | 165 | ASH6 (SEQ ID |
| | | NO: 484) | | NO: 560) |
| 338 | 187 | G3S (SEQ ID | 167 | ASH6 (SEQ ID |
| | | NO: 484) | | NO: 560) |
| 339 | 187 | G3S (SEQ ID | 170 | ASH6 (SEQ ID |
| | | NO: 484) | | NO: 560) |
| 340 | 197 | G3S (SEQ ID | 163 | ASH6 (SEQ ID |
| | | NO: 484) | | NO: 560) |
| 341 | 197 | G3S (SEQ ID | 159 | ASH6 (SEQ ID |
| | | NO: 484) | | NO: 560) |
| 342 | 197 | G3S (SEQ ID | 161 | ASH6 (SEQ ID |
| | | NO: 484) | | NO: 560) |
| 343 | 197 | G3S (SEQ ID | 162 | ASH6 (SEQ ID |
| | | NO: 484) | | NO: 560) |
| 344 | 197 | G3S (SEQ ID | 165 | ASH6 (SEQ ID |
| | | NO: 484) | | NO: 560) |
| 345 | 197 | G3S (SEQ ID | 167 | ASH6 (SEQ ID |
| | | NO: 484) | | NO: 560) |
| 346 | 197 | G3S (SEQ ID | 170 | ASH6 (SEQ ID |
| | | NO: 484) | | NO: 560) |
| 347 | 198 | G3S (SEQ ID | 163 | ASH6 (SEQ ID |
| | | NO: 484) | | NO: 560) |
| 348 | 198 | G3S (SEQ ID | 159 | ASH6 (SEQ ID |
| | | NO: 484) | | NO: 560) |
| 349 | 198 | G3S (SEQ ID | 161 | ASH6 (SEQ ID |
| | | NO: 484) | | NO: 560) |
| 350 | 198 | G3S (SEQ ID | 162 | ASH6 (SEQ ID |
| | | NO: 484) | | NO: 560) |
| 351 | 198 | G3S (SEQ ID | 165 | ASH6 (SEQ ID |
| | | NO: 484) | | NO: 560) |
| 352 | 198 | G3S (SEQ ID | 167 | ASH6 (SEQ ID |
| | | NO: 484) | | NO: 560) |
| 353 | 198 | G3S (SEQ ID | 170 | ASH6 (SEQ ID |
| | | NO: 484) | | NO: 560) |

TABLE 12
Amino Acid Sequences of IL10R Binding Molecules

| | | SEQ ID |
|---|---|---|
| NAME | Amino Acid Sequence | NO |
| 163-172 | QVQLQESGGGSVQAGGSLRLSCAASRYLYSIDYMAWFRQSPGKEREP | 256 |
| | VAVIYTASGATFYPDSVKGRFTISQDNAKMTVYLQMNSLKSEDTAMY | |
| | YCAAVRKTDSYLFDAQSFTYWGQGTQVTVSSGGGSQVQLQESGGGSV | |
| | QAGGSLRLSCAASGYTYSSGCMGWFRQAPGKEREAVAAINSDGSTSY | |
| | ADSVKGRFTISKDNAKNTLYLQMNSLKPEDTAMYYCAAEPYCSGGYP | |
| | RWSVAEFGYWGQGTQVTVSSASHHHHHH | |
| 163-183 | QVQLQESGGGSVQAGGSLRLSCAASRYLYSIDYMAWFRQSPGKEREP | 257 |
| | VAVIYTASGATFYPDSVKGRFTISQDNAKMTVYLQMNSLKSEDTAMY | |
| | YCAAVRKTDSYLFDAQSFTYWGQGTQVTVSSGGGSQVQLQESGGGSV | |
| | QAGGSLRLSCAASGYTYSSYCMGWFRQAPGKEREGVAHIDSDGSTSY | |
| | ADSVKGRFTISKDNAKNTLYLQMNSLKPEDTAMYYCAADPIPGPGYC | |
| | DGGPNKYWGQGTQVTVSSASHHHHHH | |
| 163-174 | QVQLQESGGGSVQAGGSLRLSCAASRYLYSIDYMAWFRQSPGKEREP | 258 |
| | VAVIYTASGATFYPDSVKGRFTISQDNAKMTVYLQMNSLKSEDTAMY | |
| | YCAAVRKTDSYLFDAQSFTYWGQGTQVTVSSGGGSQVQLQESGGGSV | |
| | QAGGSLRLSCAASRYTYNSYCMGWFRQAPGKEREGVATIDSDGMTRY | |
| | ADSVKGRFTISKDNAKNTLYLQMNSLKPEDTAMYYCAADADCTIAAM | |
| | TTNPLGQGTQVTVSSASHHHHHH | |
| 163-186 | QVQLQESGGGSVQAGGSLRLSCAASRYLYSIDYMAWFRQSPGKEREP | 259 |
| | VAVIYTASGATFYPDSVKGRFTISQDNAKMTVYLQMNSLKSEDTAMY | |
| | YCAAVRKTDSYLFDAQSFTYWGQGTQVTVSSGGGSQVQLQESGGGSV | |
| | QAGGSLRLSCTVSRYTASVNYMGWFRQAPGKEREGVATIFTGAGTTY | |
| | YANSVKGRFTISRDNAKNTAYLQMNSLKPEDTAIYYCAVDFRGGLLY | |
| | RPAYEYTYRGQGTQVTVSSASHHHHHH | |
| 163-187 | QVQLQESGGGSVQAGGSLRLSCAASRYLYSIDYMAWFRQSPGKEREP | 260 |
| | VAVIYTASGATFYPDSVKGRFTISQDNAKMTVYLQMNSLKSEDTAMY | |
| | YCAAVRKTDSYLFDAQSFTYWGQGTQVTVSSGGGSQVQLQESGGGSV | |
| | EAGGSLRLSCAASGYTHSSYCMGWFRQAPGKEREGVAAIDVDGSTTY | |
| | ADSVKGRFTISKDNAKNTLYLQMNSLKPEDTGMYYCAAEFADCSSNY | |
| | FLPPGAVRYWGQGTQVTVSSASHHHHHH | |
| 163-197 | QVQLQESGGGSVQAGGSLRLSCAASRYLYSIDYMAWFRQSPGKEREP | 261 |
| | VAVIYTASGATFYPDSVKGRFTISQDNAKMTVYLQMNSLKSEDTAMY | |
| | YCAAVRKTDSYLFDAQSFTYWGQGTQVTVSSGGGSQVQLQESGGGSV | |
| | QAGGSLRLSCAASGYSYSSYCMGWFRQAPGKEREGVATIDSDGMTRY | |
| | ADSVKGRFTISKDNAKNTLYLQMNSLKPEDTAMYYCAAPLYDCDSGA | |
| | VGRNPPYWGQGTQVTVSSASHHHHHH | |
| 163-198 | QVQLQESGGGSVQAGGSLRLSCAASRYLYSIDYMAWFRQSPGKEREP | 262 |
| | VAVIYTASGATFYPDSVKGRFTISQDNAKMTVYLQMNSLKSEDTAMY | |
| | YCAAVRKTDSYLFDAQSFTYWGQGTQVTVSSGGGSQVQLQESGGGSV | |
| | QTGGSLRLSCAASGYTYLRGCMGWFRQAPGKEREGVAVMDVVGDRRS | |
| | YIDSVKGRFTISRDNAANSVYLQMDNLKPEDTAMYYCTAGPNCVGWR | |
| | SGLDYWGQGTQVTVSSASHHHHHH | |
| 159-172 | QVQLQESGGGSVQAGGSLRLSCAASRFTYSSYCMGWFRQAPGKEREG | 263 |
| | VASIDSDGSTSYTDSVKGRFTISKDNAKNTLYLQMNSLKPEDTAMYY | |
| | CALDLMSTVVPGFCGFLLSAGMDYWGKGTQVTVSSGGGSQVQLQESG | |
| | GGSVQAGGSLRLSCAASGYTYSSGCMGWFRQAPGKEREAVAAINSDG | |
| | STSYADSVKGRFTISKDNAKNTLYLQMNSLKPEDTAMYYCAAEPYCS | |
| | GGYPRWSVAEFGYWGQGTQVTVSSASHHHHHH | |
| 159-183 | QVQLQESGGGSVQAGGSLRLSCAASRFTYSSYCMGWFRQAPGKEREG | 264 |
| | VASIDSDGSTSYTDSVKGRFTISKDNAKNTLYLQMNSLKPEDTAMYY | |
| | CALDLMSTVVPGFCGFLLSAGMDYWGKGTQVTVSSGGGSQVQLQESG | |
| | GGSVQAGGSLRLSCAASGYTYSSYCMGWFRQAPGKEREGVAHIDSDG | |
| | STSYADSVKGRFTISKDNAKNTLYLQMNSLKPEDTAMYYCAADPIPG | |
| | PGYCDGGPNKYWGQGTQVTVSSASHHHHHH | |
| 159-174 | QVQLQESGGGSVQAGGSLRLSCAASRFTYSSYCMGWFRQAPGKEREG | 265 |
| | VASIDSDGSTSYTDSVKGRFTISKDNAKNTLYLQMNSLKPEDTAMYY | |
| | CALDLMSTVVPGFCGFLLSAGMDYWGKGTQVTVSSGGGSQVQLQESG | |
| | GGSVQAGGSLRLSCAASRYTYNSYCMGWFRQAPGKEREGVATIDSDG | |
| | MTRYADSVKGRFTISKDNAKNTLYLQMNSLKPEDTAMYYCAADADCT | |
| | IAAMTTNPLGQGTQVTVSSASHHHHHH | |
| 159-186 | QVQLQESGGGSVQAGGSLRLSCAASRFTYSSYCMGWFRQAPGKEREG | 266 |
| | VASIDSDGSTSYTDSVKGRFTISKDNAKNTLYLQMNSLKPEDTAMYY | |
| | CALDLMSTVVPGFCGFLLSAGMDYWGKGTQVTVSSGGGSQVQLQESG | |
| | GGSVQAGGSLRLSCTVSRYTASVNYMGWFRQAPGKEREGVATIFTGA | |
| | GTTYYANSVKGRFTISRDNAKNTAYLQMNSLKPEDTAIYYCAVDFRG | |
| | GLLYRPAYEYTYRGQGTQVTVSSASHHHHHH | |
| 159-187 | QVQLQESGGGSVQAGGSLRLSCAASRFTYSSYCMGWFRQAPGKEREG | 267 |
| | VASIDSDGSTSYTDSVKGRFTISKDNAKNTLYLQMNSLKPEDTAMYY | |
| | CALDLMSTVVPGFCGFLLSAGMDYWGKGTQVTVSSGGGSQVQLQESG | |
| | GGSVEAGGSLRLSCAASGYTHSSYCMGWFRQAPGKEREGVAAIDVDG | |
| | STTYADSVKGRFTISKDNAKNTLYLQMNSLKPEDTGMYYCAAEFADC | |
| | SSNYFLPPGAVRYWGQGTQVTVSSASHHHHHH | |
| 159-197 | QVQLQESGGGSVQAGGSLRLSCAASRFTYSSYCMGWFRQAPGKEREG | 268 |
| | VASIDSDGSTSYTDSVKGRFTISKDNAKNTLYLQMNSLKPEDTAMYY | |
| | CALDLMSTVVPGFCGFLLSAGMDYWGKGTQVTVSSGGGSQVQLQESG | |
| | GGSVQAGGSLRLSCAASGYSYSSYCMGWFRQAPGKEREGVATIDSDG | |
| | MTRYADSVKGRFTISKDNAKNTLYLQMNSLKPEDTAMYYCAAPLYDC | |
| | DSGAVGRNPPYWGQGTQVTVSSASHHHHHH | |
| 159-198 | QVQLQESGGGSVQAGGSLRLSCAASRFTYSSYCMGWFRQAPGKEREG | 269 |
| | VASIDSDGSTSYTDSVKGRFTISKDNAKNTLYLQMNSLKPEDTAMYY | |
| | CALDLMSTVVPGFCGFLLSAGMDYWGKGTQVTVSSGGGSQVQLQESG | |
| | GGSVQTGGSLRLSCAASGYTYLRGCMGWFRQAPGKEREGVAVMDVVG | |
| | DRRSYIDSVKGRFTISRDNAANSVYLQMDNLKPEDTAMYYCTAGPNC | |
| | VGWRSGLDYWGQGTQVTVSSASHHHHHH | |
| 161-172 | QVQLQESGGGSVQAGGSLRLSCAASGYTYSMYCMGWFRQAPGKEREG | 270 |
| | VAQINSDGSTSYADSVKGRFTISKDNAKNTLYLQMNSLKPEDTAMYY | |
| | CAADSRVYGGSWYERLCGPYTYEYNYWGQGTQVTVSSGGGSQVQLQE | |
| | SGGGSVQAGGSLRLSCAASGYTYSSGCMGWFRQAPGKEREAVAAINS | |
| | DGSTSYADSVKGRFTISKDNAKNTLYLQMNSLKPEDTAMYYCAAEPY | |
| | CSGGYPRWSVAEFGYWGQGTQVTVSSASHHHHHH | |
| 161-183 | QVQLQESGGGSVQAGGSLRLSCAASGYTYSMYCMGWFRQAPGKEREG | 271 |
| | VAQINSDGSTSYADSVKGRFTISKDNAKNTLYLQMNSLKPEDTAMYY | |
| | CAADSRVYGGSWYERLCGPYTYEYNYWGQGTQVTVSSGGGSQVQLQE | |
| | SGGGSVQAGGSLRLSCAASGYTYSSYCMGWFRQAPGKEREGVAHIDS | |
| | DGSTSYADSVKGRFTISKDNAKNTLYLQMNSLKPEDTAMYYCAADPI | |
| | PGPGYCDGGPNKYWGQGTQVTVSSASHHHHHH | |
| 161-174 | QVQLQESGGGSVQAGGSLRLSCAASGYTYSMYCMGWFRQAPGKEREG | 272 |
| | VAQINSDGSTSYADSVKGRFTISKDNAKNTLYLQMNSLKPEDTAMYY | |
| | CAADSRVYGGSWYERLCGPYTYEYNYWGQGTQVTVSSGGGSQVQLQE | |
| | SGGGSVQAGGSLRLSCAASRYTYNSYCMGWFRQAPGKEREGVATIDS | |
| | DGMTRYADSVKGRFTISKDNAKNTLYLQMNSLKPEDTAMYYCAADAD | |
| | CTIAAMTTNPLGQGTQVTVSSASHHHHHH | |
| 161-186 | QVQLQESGGGSVQAGGSLRLSCAASGYTYSMYCMGWFRQAPGKEREG | 273 |
| | VAQINSDGSTSYADSVKGRFTISKDNAKNTLYLQMNSLKPEDTAMYY | |
| | CAADSRVYGGSWYERLCGPYTYEYNYWGQGTQVTVSSGGGSQVQLQE | |
| | SGGGSVQAGGSLRLSCTVSRYTASVNYMGWFRQAPGKEREGVATIFT | |
| | GAGTTYYANSVKGRFTISRDNAKNTAYLQMNSLKPEDTAIYYCAVDF | |
| | RGGLLYRPAYEYTYRGQGTQVTVSSASHHHHHH | |
| 161-187 | QVQLQESGGGSVQAGGSLRLSCAASGYTYSMYCMGWFRQAPGKEREG | 274 |
| | VAQINSDGSTSYADSVKGRFTISKDNAKNTLYLQMNSLKPEDTAMYY | |
| | CAADSRVYGGSWYERLCGPYTYEYNYWGQGTQVTVSSGGGSQVQLQE | |
| | SGGGSVEAGGSLRLSCAASGYTHSSYCMGWFRQAPGKEREGVAAIDV | |
| | DGSTTYADSVKGRFTISKDNAKNTLYLQMNSLKPEDTGMYYCAAEFA | |
| | DCSSNYFLPPGAVRYWGQGTQVTVSSASHHHHHH | |
| 161-197 | QVQLQESGGGSVQAGGSLRLSCAASGYTYSMYCMGWFRQAPGKEREG | 275 |
| | VAQINSDGSTSYADSVKGRFTISKDNAKNTLYLQMNSLKPEDTAMYY | |
| | CAADSRVYGGSWYERLCGPYTYEYNYWGQGTQVTVSSGGGSQVQLQE | |
| | SGGGSVQAGGSLRLSCAASGYSYSSYCMGWFRQAPGKEREGVATIDS | |
| | DGMTRYADSVKGRFTISKDNAKNTLYLQMNSLKPEDTAMYYCAAPLY | |
| | DCDSGAVGRNPPYWGQGTQVTVSSASHHHHHH | |
| 161-198 | QVQLQESGGGSVQAGGSLRLSCAASGYTYSMYCMGWFRQAPGKEREG | 276 |
| | VAQINSDGSTSYADSVKGRFTISKDNAKNTLYLQMNSLKPEDTAMYY | |
| | CAADSRVYGGSWYERLCGPYTYEYNYWGQGTQVTVSSGGGSQVQLQE | |
| | SGGGSVQTGGSLRLSCAASGYTYLRGCMGWFRQAPGKEREGVAVMDV | |
| | VGDRRSYIDSVKGRFTISRDNAANSVYLQMDNLKPEDTAMYYCTAGP | |
| | NCVGWRSGLDYWGQGTQVTVSSASHHHHHH | |
| 162-172 | QVQLQESGGGSVQAGGSLRLSCAVSGYAYSTYCMGWFRQAPGKEREG | 277 |
| | VAAIDSGGSTSYADSVKGRFTISKDNAKNTLYLRMNSLKPEDTAMYY | |
| | CAAVPPPPDGGSCLFLGPEIKVSKADFRYWGQGTQVTVSSGGGSQVQ | |
| | LQESGGGSVQAGGSLRLSCAASGYTYSSGCMGWFRQAPGKEREAVAA | |
| | INSDGSTSYADSVKGRFTISKDNAKNTLYLQMNSLKPEDTAMYYCAA | |
| | EPYCSGGYPRWSVAEFGYWGQGTQVTVSSASHHHHHH | |
| 162-183 | QVQLQESGGGSVQAGGSLRLSCAVSGYAYSTYCMGWFRQAPGKEREG | 278 |
| | VAAIDSGGSTSYADSVKGRFTISKDNAKNTLYLRMNSLKPEDTAMYY | |
| | CAAVPPPPDGGSCLFLGPEIKVSKADFRYWGQGTQVTVSSGGGSQVQ | |
| | LQESGGGSVQAGGSLRLSCAASGYTYSSYCMGWFRQAPGKEREGVAH | |
| | IDSDGSTSYADSVKGRFTISKDNAKNTLYLQMNSLKPEDTAMYYCAA | |
| | DPIPGPGYCDGGPNKYWGQGTQVTVSSASHHHHHH | |
| 162-174 | QVQLQESGGGSVQAGGSLRLSCAVSGYAYSTYCMGWFRQAPGKEREG | 279 |
| | VAAIDSGGSTSYADSVKGRFTISKDNAKNTLYLRMNSLKPEDTAMYY | |
| | CAAVPPPPDGGSCLFLGPEIKVSKADFRYWGQGTQVTVSSGGGSQVQ | |
| | LQESGGGSVQAGGSLRLSCAASRYTYNSYCMGWFRQAPGKEREGVAT | |
| | IDSDGMTRYADSVKGRFTISKDNAKNTLYLQMNSLKPEDTAMYYCAA | |
| | DADCTIAAMTTNPLGQGTQVTVSSASHHHHHH | |
| 162-186 | QVQLQESGGGSVQAGGSLRLSCAVSGYAYSTYCMGWFRQAPGKEREG | 280 |
| | VAAIDSGGSTSYADSVKGRFTISKDNAKNTLYLRMNSLKPEDTAMYY | |
| | CAAVPPPPDGGSCLFLGPEIKVSKADFRYWGQGTQVTVSSGGGSQVQ | |
| | LQESGGGSVQAGGSLRLSCTVSRYTASVNYMGWFRQAPGKEREGVAT | |
| | IFTGAGTTYYANSVKGRFTISRDNAKNTAYLQMNSLKPEDTAIYYCA | |
| | VDFRGGLLYRPAYEYTYRGQGTQVTVSSASHHHHHH | |
| 162-187 | QVQLQESGGGSVQAGGSLRLSCAVSGYAYSTYCMGWFRQAPGKEREG | 281 |
| | VAAIDSGGSTSYADSVKGRFTISKDNAKNTLYLRMNSLKPEDTAMYY | |
| | CAAVPPPPDGGSCLFLGPEIKVSKADFRYWGQGTQVTVSSGGGSQVQ | |
| | LQESGGGSVEAGGSLRLSCAASGYTHSSYCMGWFRQAPGKEREGVAA | |
| | IDVDGSTTYADSVKGRFTISKDNAKNTLYLQMNSLKPEDTGMYYCAA | |
| | EFADCSSNYFLPPGAVRYWGQGTQVTVSSASHHHHHH | |
| 162-197 | QVQLQESGGGSVQAGGSLRLSCAVSGYAYSTYCMGWFRQAPGKEREG | 282 |
| | VAAIDSGGSTSYADSVKGRFTISKDNAKNTLYLRMNSLKPEDTAMYY | |
| | CAAVPPPPDGGSCLFLGPEIKVSKADFRYWGQGTQVTVSSGGGSQVQ | |
| | LQESGGGSVQAGGSLRLSCAASGYSYSSYCMGWFRQAPGKEREGVAT | |
| | IDSDGMTRYADSVKGRFTISKDNAKNTLYLQMNSLKPEDTAMYYCAA | |
| | PLYDCDSGAVGRNPPYWGQGTQVTVSSASHHHHHH | |
| 162-198 | QVQLQESGGGSVQAGGSLRLSCAVSGYAYSTYCMGWFRQAPGKEREG | 283 |
| | VAAIDSGGSTSYADSVKGRFTISKDNAKNTLYLRMNSLKPEDTAMYY | |
| | CAAVPPPPDGGSCLFLGPEIKVSKADFRYWGQGTQVTVSSGGGSQVQ | |
| | LQESGGGSVQTGGSLRLSCAASGYTYLRGCMGWFRQAPGKEREGVAV | |
| | MDVVGDRRSYIDSVKGRFTISRDNAANSVYLQMDNLKPEDTAMYYCT | |
| | AGPNCVGWRSGLDYWGQGTQVTVSSASHHHHHH | |
| 165-172 | QVQLQESGGGSVQAGGSLRLSCTVSGYTYSSNCMGWFRQAPGKEREG | 284 |
| | VATIYTGGGNTYYADSVKGRFTISQDNAKNTVYLQMNNLKPEDTAMY | |
| | YCAAEPLSRVYGGSCPTPTFDYWGQGTQVTVSSGGGSQVQLQESGGG | |
| | SVQAGGSLRLSCAASGYTYSSGCMGWFRQAPGKEREAVAAINSDGST | |
| | SYADSVKGRFTISKDNAKNTLYLQMNSLKPEDTAMYYCAAEPYCSGG | |
| | YPRWSVAEFGYWGQGTQVTVSSASHHHHHH | |
| 165-183 | QVQLQESGGGSVQAGGSLRLSCTVSGYTYSSNCMGWFRQAPGKEREG | 285 |
| | VATIYTGGGNTYYADSVKGRFTISQDNAKNTVYLQMNNLKPEDTAMY | |
| | YCAAEPLSRVYGGSCPTPTFDYWGQGTQVTVSSGGGSQVQLQESGGG | |
| | SVQAGGSLRLSCAASGYTYSSYCMGWFRQAPGKEREGVAHIDSDGST | |
| | SYADSVKGRFTISKDNAKNTLYLQMNSLKPEDTAMYYCAADPIPGPG | |
| | YCDGGPNKYWGQGTQVTVSSASHHHHHH | |
| 165-174 | QVQLQESGGGSVQAGGSLRLSCTVSGYTYSSNCMGWFRQAPGKEREG | 286 |
| | VATIYTGGGNTYYADSVKGRFTISQDNAKNTVYLQMNNLKPEDTAMY | |
| | YCAAEPLSRVYGGSCPTPTFDYWGQGTQVTVSSGGGSQVQLQESGGG | |
| | SVQAGGSLRLSCAASRYTYNSYCMGWFRQAPGKEREGVATIDSDGMT | |
| | RYADSVKGRFTISKDNAKNTLYLQMNSLKPEDTAMYYCAADADCTIA | |
| | AMTTNPLGQGTQVTVSSASHHHHHH | |
| 165-186 | QVQLQESGGGSVQAGGSLRLSCTVSGYTYSSNCMGWFRQAPGKEREG | 287 |
| | VATIYTGGGNTYYADSVKGRFTISQDNAKNTVYLQMNNLKPEDTAMY | |
| | YCAAEPLSRVYGGSCPTPTFDYWGQGTQVTVSSGGGSQVQLQESGGG | |
| | SVQAGGSLRLSCTVSRYTASVNYMGWFRQAPGKEREGVATIFTGAGT | |
| | TYYANSVKGRFTISRDNAKNTAYLQMNSLKPEDTAIYYCAVDFRGGL | |
| | LYRPAYEYTYRGQGTQVTVSSASHHHHHH | |
| 165-187 | QVQLQESGGGSVQAGGSLRLSCTVSGYTYSSNCMGWFRQAPGKEREG | 288 |
| | VATIYTGGGNTYYADSVKGRFTISQDNAKNTVYLQMNNLKPEDTAMY | |
| | YCAAEPLSRVYGGSCPTPTFDYWGQGTQVTVSSGGGSQVQLQESGGG | |
| | SVEAGGSLRLSCAASGYTHSSYCMGWFRQAPGKEREGVAAIDVDGST | |
| | TYADSVKGRFTISKDNAKNTLYLQMNSLKPEDTGMYYCAAEFADCSS | |
| | NYFLPPGAVRYWGQGTQVTVSSASHHHHHH | |
| 165-197 | QVQLQESGGGSVQAGGSLRLSCTVSGYTYSSNCMGWFRQAPGKEREG | 289 |
| | VATIYTGGGNTYYADSVKGRFTISQDNAKNTVYLQMNNLKPEDTAMY | |
| | YCAAEPLSRVYGGSCPTPTFDYWGQGTQVTVSSGGGSQVQLQESGGG | |
| | SVQAGGSLRLSCAASGYSYSSYCMGWFRQAPGKEREGVATIDSDGMT | |
| | RYADSVKGRFTISKDNAKNTLYLQMNSLKPEDTAMYYCAAPLYDCDS | |
| | GAVGRNPPYWGQGTQVTVSSASHHHHHH | |
| 165-198 | QVQLQESGGGSVQAGGSLRLSCTVSGYTYSSNCMGWFRQAPGKEREG | 290 |
| DR509 | VATIYTGGGNTYYADSVKGRFTISQDNAKNTVYLQMNNLKPEDTAMY | |
| | YCAAEPLSRVYGGSCPTPTFDYWGQGTQVTVSSGGGSQVQLQESGGG | |
| | SVQTGGSLRLSCAASGYTYLRGCMGWFRQAPGKEREGVAVMDVVGDR | |
| | RSYIDSVKGRFTISRDNAANSVYLQMDNLKPEDTAMYYCTAGPNCVG | |
| | WRSGLDYWGQGTQVTVSSASHHHHHH | |
| 167-172 | QVQLQESGGGSVQAGGSLRLSCGASGYTYSSYCMGWFRQVPGKEREG | 291 |
| | VAVIDSDGSTSYADSVKGRFTISKDNGKNTLYLQMNSLKPEDTAMYY | |
| | CAADLGHYRPPCGVLYLGMDYWGKGTQVTVSSGGGSQVQLQESGGGS | |
| | VQAGGSLRLSCAASGYTYSSGCMGWFRQAPGKEREAVAAINSDGSTS | |
| | YADSVKGRFTISKDNAKNTLYLQMNSLKPEDTAMYYCAAEPYCSGGY | |
| | PRWSVAEFGYWGQGTQVTVSSASHHHHHH | |
| 167-183 | QVQLQESGGGSVQAGGSLRLSCGASGYTYSSYCMGWFRQVPGKEREG | 292 |
| | VAVIDSDGSTSYADSVKGRFTISKDNGKNTLYLQMNSLKPEDTAMYY | |
| | CAADLGHYRPPCGVLYLGMDYWGKGTQVTVSSGGGSQVQLQESGGGS | |
| | VQAGGSLRLSCAASGYTYSSYCMGWFRQAPGKEREGVAHIDSDGSTS | |
| | YADSVKGRFTISKDNAKNTLYLQMNSLKPEDTAMYYCAADPIPGPGY | |
| | CDGGPNKYWGQGTQVTVSSASHHHHHH | |
| 167-174 | QVQLQESGGGSVQAGGSLRLSCGASGYTYSSYCMGWFRQVPGKEREG | 293 |
| | VAVIDSDGSTSYADSVKGRFTISKDNGKNTLYLQMNSLKPEDTAMYY | |
| | CAADLGHYRPPCGVLYLGMDYWGKGTQVTVSSGGGSQVQLQESGGGS | |
| | VQAGGSLRLSCAASRYTYNSYCMGWFRQAPGKEREGVATIDSDGMTR | |
| | YADSVKGRFTISKDNAKNTLYLQMNSLKPEDTAMYYCAADADCTIAA | |
| | MTTNPLGQGTQVTVSSASHHHHHH | |
| 167-186 | QVQLQESGGGSVQAGGSLRLSCGASGYTYSSYCMGWFRQVPGKEREG | 294 |
| | VAVIDSDGSTSYADSVKGRFTISKDNGKNTLYLQMNSLKPEDTAMYY | |
| | CAADLGHYRPPCGVLYLGMDYWGKGTQVTVSSGGGSQVQLQESGGGS | |
| | VQAGGSLRLSCTVSRYTASVNYMGWFRQAPGKEREGVATIFTGAGTT | |
| | YYANSVKGRFTISRDNAKNTAYLQMNSLKPEDTAIYYCAVDFRGGLL | |
| | YRPAYEYTYRGQGTQVTVSSASHHHHHH | |
| 167-187 | QVQLQESGGGSVQAGGSLRLSCGASGYTYSSYCMGWFRQVPGKEREG | 295 |
| | VAVIDSDGSTSYADSVKGRFTISKDNGKNTLYLQMNSLKPEDTAMYY | |
| | CAADLGHYRPPCGVLYLGMDYWGKGTQVTVSSGGGSQVQLQESGGGS | |
| | VEAGGSLRLSCAASGYTHSSYCMGWFRQAPGKEREGVAAIDVDGSTT | |
| | YADSVKGRFTISKDNAKNTLYLQMNSLKPEDTGMYYCAAEFADCSSN | |
| | YFLPPGAVRYWGQGTQVTVSSASHHHHHH | |
| 167-197 | QVQLQESGGGSVQAGGSLRLSCGASGYTYSSYCMGWFRQVPGKEREG | 296 |
| | VAVIDSDGSTSYADSVKGRFTISKDNGKNTLYLQMNSLKPEDTAMYY | |
| | CAADLGHYRPPCGVLYLGMDYWGKGTQVTVSSGGGSQVQLQESGGGS | |
| | VQAGGSLRLSCAASGYSYSSYCMGWFRQAPGKEREGVATIDSDGMTR | |
| | YADSVKGRFTISKDNAKNTLYLQMNSLKPEDTAMYYCAAPLYDCDSG | |
| | AVGRNPPYWGQGTQVTVSSASHHHHHH | |
| 167-198 | QVQLQESGGGSVQAGGSLRLSCGASGYTYSSYCMGWFRQVPGKEREG | 297 |
| | VAVIDSDGSTSYADSVKGRFTISKDNGKNTLYLQMNSLKPEDTAMYY | |
| | CAADLGHYRPPCGVLYLGMDYWGKGTQVTVSSGGGSQVQLQESGGGS | |
| | VQTGGSLRLSCAASGYTYLRGCMGWFRQAPGKEREGVAVMDVVGDRR | |
| | SYIDSVKGRFTISRDNAANSVYLQMDNLKPEDTAMYYCTAGPNCVGW | |
| | RSGLDYWGQGTQVTVSSASHHHHHH | |
| 170-172 | QVQLQESGGGSVQAGGSLRLSCAASGYSNCSYDMTWYRQAPGKEREF | 298 |
| | VSAIHSDGSTRYADSVKGRFFISQDNAKNTVYLQMNSLKPEDTAMYY | |
| | CKTDPLHCRAHGGSWYSVRANYWGQGTQVTVSSGGGSQVQLQESGGG | |
| | SVQAGGSLRLSCAASGYTYSSGCMGWFRQAPGKEREAVAAINSDGST | |
| | SYADSVKGRFTISKDNAKNTLYLQMNSLKPEDTAMYYCAAEPYCSGG | |
| | YPRWSVAEFGYWGQGTQVTVSSASHHHHHH | |
| 170-183 | QVQLQESGGGSVQAGGSLRLSCAASGYSNCSYDMTWYRQAPGKEREF | 299 |
| | VSAIHSDGSTRYADSVKGRFFISQDNAKNTVYLQMNSLKPEDTAMYY | |
| | CKTDPLHCRAHGGSWYSVRANYWGQGTQVTVSSGGGSQVQLQESGGG | |
| | SVQAGGSLRLSCAASGYTYSSYCMGWFRQAPGKEREGVAHIDSDGST | |
| | SYADSVKGRFTISKDNAKNTLYLQMNSLKPEDTAMYYCAADPIPGPG | |
| | YCDGGPNKYWGQGTQVTVSSASHHHHHH | |
| 170-174 | QVQLQESGGGSVQAGGSLRLSCAASGYSNCSYDMTWYRQAPGKEREF | 300 |
| | VSAIHSDGSTRYADSVKGRFFISQDNAKNTVYLQMNSLKPEDTAMYY | |
| | CKTDPLHCRAHGGSWYSVRANYWGQGTQVTVSSGGGSQVQLQESGGG | |
| | SVQAGGSLRLSCAASRYTYNSYCMGWFRQAPGKEREGVATIDSDGMT | |
| | RYADSVKGRFTISKDNAKNTLYLQMNSLKPEDTAMYYCAADADCTIA | |
| | AMTTNPLGQGTQVTVSSASHHHHHH | |
| 170-186 | QVQLQESGGGSVQAGGSLRLSCAASGYSNCSYDMTWYRQAPGKEREF | 301 |
| | VSAIHSDGSTRYADSVKGRFFISQDNAKNTVYLQMNSLKPEDTAMYY | |
| | CKTDPLHCRAHGGSWYSVRANYWGQGTQVTVSSGGGSQVQLQESGGG | |
| | SVQAGGSLRLSCTVSRYTASVNYMGWFRQAPGKEREGVATIFTGAGT | |
| | TYYANSVKGRFTISRDNAKNTAYLQMNSLKPEDTAIYYCAVDFRGGL | |
| | LYRPAYEYTYRGQGTQVTVSSASHHHHHH | |
| 170-187 | QVQLQESGGGSVQAGGSLRLSCAASGYSNCSYDMTWYRQAPGKEREF | 302 |
| DR521 | VSAIHSDGSTRYADSVKGRFFISQDNAKNTVYLQMNSLKPEDTAMYY | |
| | CKTDPLHCRAHGGSWYSVRANYWGQGTQVTVSSGGGSQVQLQESGGG | |
| | SVEAGGSLRLSCAASGYTHSSYCMGWFRQAPGKEREGVAAIDVDGST | |
| | TYADSVKGRFTISKDNAKNTLYLQMNSLKPEDTGMYYCAAEFADCSS | |
| | NYFLPPGAVRYWGQGTQVTVSSASHHHHHH | |
| 170-197 | QVQLQESGGGSVQAGGSLRLSCAASGYSNCSYDMTWYRQAPGKEREF | 303 |
| | VSAIHSDGSTRYADSVKGRFFISQDNAKNTVYLQMNSLKPEDTAMYY | |
| | CKTDPLHCRAHGGSWYSVRANYWGQGTQVTVSSGGGSQVQLQESGGG | |
| | SVQAGGSLRLSCAASGYSYSSYCMGWFRQAPGKEREGVATIDSDGMT | |
| | RYADSVKGRFTISKDNAKNTLYLQMNSLKPEDTAMYYCAAPLYDCDS | |
| | GAVGRNPPYWGQGTQVTVSSASHHHHHH | |
| 170-198 | QVQLQESGGGSVQAGGSLRLSCAASGYSNCSYDMTWYRQAPGKEREF | 304 |
| | VSAIHSDGSTRYADSVKGRFFISQDNAKNTVYLQMNSLKPEDTAMYY | |
| | CKTDPLHCRAHGGSWYSVRANYWGQGTQVTVSSGGGSQVQLQESGGG | |
| | SVQTGGSLRLSCAASGYTYLRGCMGWFRQAPGKEREGVAVMDVVGDR | |
| | RSYIDSVKGRFTISRDNAANSVYLQMDNLKPEDTAMYYCTAGPNCVG | |
| | WRSGLDYWGQGTQVTVSSASHHHHHH | |
| 172-163 | QVQLQESGGGSVQAGGSLRLSCAASGYTYSSGCMGWFRQAPGKEREA | 305 |
| | VAAINSDGSTSYADSVKGRFTISKDNAKNTLYLQMNSLKPEDTAMYY | |
| | CAAEPYCSGGYPRWSVAEFGYWGQGTQVTVSSGGGSQVQLQESGGGS | |
| | VQAGGSLRLSCAASRYLYSIDYMAWFRQSPGKEREPVAVIYTASGAT | |
| | FYPDSVKGRFTISQDNAKMTVYLQMNSLKSEDTAMYYCAAVRKTDSY | |
| | LFDAQSFTYWGQGTQVTVSSASHHHHHH | |
| 172-159 | QVQLQESGGGSVQAGGSLRLSCAASGYTYSSGCMGWFRQAPGKEREA | 306 |
| | VAAINSDGSTSYADSVKGRFTISKDNAKNTLYLQMNSLKPEDTAMYY | |
| | CAAEPYCSGGYPRWSVAEFGYWGQGTQVTVSSGGGSQVQLQESGGGS | |
| | VQAGGSLRLSCAASRFTYSSYCMGWFRQAPGKEREGVASIDSDGSTS | |
| | YTDSVKGRFTISKDNAKNTLYLQMNSLKPEDTAMYYCALDLMSTVVP | |
| | GFCGFLLSAGMDYWGKGTQVTVSSASHHHHHH | |
| 172-161 | QVQLQESGGGSVQAGGSLRLSCAASGYTYSSGCMGWFRQAPGKEREA | 307 |
| | VAAINSDGSTSYADSVKGRFTISKDNAKNTLYLQMNSLKPEDTAMYY | |
| | CAAEPYCSGGYPRWSVAEFGYWGQGTQVTVSSGGGSQVQLQESGGGS | |
| | VQAGGSLRLSCAASGYTYSMYCMGWFRQAPGKEREGVAQINSDGSTS | |
| | YADSVKGRFTISKDNAKNTLYLQMNSLKPEDTAMYYCAADSRVYGGS | |
| | WYERLCGPYTYEYNYWGQGTQVTVSSASHHHHHH | |
| 172-162 | QVQLQESGGGSVQAGGSLRLSCAASGYTYSSGCMGWFRQAPGKEREA | 308 |
| DR527 | VAAINSDGSTSYADSVKGRFTISKDNAKNTLYLQMNSLKPEDTAMYY | |
| | CAAEPYCSGGYPRWSVAEFGYWGQGTQVTVSSGGGSQVQLQESGGGS | |
| | VQAGGSLRLSCAVSGYAYSTYCMGWFRQAPGKEREGVAAIDSGGSTS | |
| | YADSVKGRFTISKDNAKNTLYLRMNSLKPEDTAMYYCAAVPPPPDGG | |
| | SCLFLGPEIKVSKADFRYWGQGTQVTVSSASHHHHHH | |
| 172-165 | QVQLQESGGGSVQAGGSLRLSCAASGYTYSSGCMGWFRQAPGKEREA | 309 |
| | VAAINSDGSTSYADSVKGRFTISKDNAKNTLYLQMNSLKPEDTAMYY | |
| | CAAEPYCSGGYPRWSVAEFGYWGQGTQVTVSSGGGSQVQLQESGGGS | |
| | VQAGGSLRLSCTVSGYTYSSNCMGWFRQAPGKEREGVATIYTGGGNT | |
| | YYADSVKGRFTISQDNAKNTVYLQMNNLKPEDTAMYYCAAEPLSRVY | |
| | GGSCPTPTFDYWGQGTQVTVSSASHHHHHH | |
| 172-167 | QVQLQESGGGSVQAGGSLRLSCAASGYTYSSGCMGWFRQAPGKEREA | 310 |
| | VAAINSDGSTSYADSVKGRFTISKDNAKNTLYLQMNSLKPEDTAMYY | |
| | CAAEPYCSGGYPRWSVAEFGYWGQGTQVTVSSGGGSQVQLQESGGGS | |
| | VQAGGSLRLSCGASGYTYSSYCMGWFRQVPGKEREGVAVIDSDGSTS | |
| | YADSVKGRFTISKDNGKNTLYLQMNSLKPEDTAMYYCAADLGHYRPP | |
| | CGVLYLGMDYWGKGTQVTVSSASHHHHHH | |
| 172-170 | QVQLQESGGGSVQAGGSLRLSCAASGYTYSSGCMGWFRQAPGKEREA | 311 |
| | VAAINSDGSTSYADSVKGRFTISKDNAKNTLYLQMNSLKPEDTAMYY | |
| | CAAEPYCSGGYPRWSVAEFGYWGQGTQVTVSSGGGSQVQLQESGGGS | |
| | VQAGGSLRLSCAASGYSNCSYDMTWYRQAPGKEREFVSAIHSDGSTR | |
| | YADSVKGRFFISQDNAKNTVYLQMNSLKPEDTAMYYCKTDPLHCRAH | |
| | GGSWYSVRANYWGQGTQVTVSSASHHHHHH | |
| 183-163 | QVQLQESGGGSVQAGGSLRLSCAASGYTYSSYCMGWFRQAPGKEREG | 312 |
| | VAHIDSDGSTSYADSVKGRFTISKDNAKNTLYLQMNSLKPEDTAMYY | |
| | CAADPIPGPGYCDGGPNKYWGQGTQVTVSSGGGSQVQLQESGGGSVQ | |
| | AGGSLRLSCAASRYLYSIDYMAWFRQSPGKEREPVAVIYTASGATFY | |
| | PDSVKGRFTISQDNAKMTVYLQMNSLKSEDTAMYYCAAVRKTDSYLF | |
| | DAQSFTYWGQGTQVTVSSASHHHHHH | |
| 183-159 | QVQLQESGGGSVQAGGSLRLSCAASGYTYSSYCMGWFRQAPGKEREG | 313 |
| | VAHIDSDGSTSYADSVKGRFTISKDNAKNTLYLQMNSLKPEDTAMYY | |
| | CAADPIPGPGYCDGGPNKYWGQGTQVTVSSGGGSQVQLQESGGGSVQ | |
| | AGGSLRLSCAASRFTYSSYCMGWFRQAPGKEREGVASIDSDGSTSYT | |
| | DSVKGRFTISKDNAKNTLYLQMNSLKPEDTAMYYCALDLMSTVVPGF | |
| | CGFLLSAGMDYWGKGTQVTVSSASHHHHHH | |
| 183-161 | QVQLQESGGGSVQAGGSLRLSCAASGYTYSSYCMGWFRQAPGKEREG | 314 |
| | VAHIDSDGSTSYADSVKGRFTISKDNAKNTLYLQMNSLKPEDTAMYY | |
| | CAADPIPGPGYCDGGPNKYWGQGTQVTVSSGGGSQVQLQESGGGSVQ | |
| | AGGSLRLSCAASGYTYSMYCMGWFRQAPGKEREGVAQINSDGSTSYA | |
| | DSVKGRFTISKDNAKNTLYLQMNSLKPEDTAMYYCAADSRVYGGSWY | |
| | ERLCGPYTYEYNYWGQGTQVTVSSASHHHHHH | |
| 183-162 | QVQLQESGGGSVQAGGSLRLSCAASGYTYSSYCMGWFRQAPGKEREG | 315 |
| | VAHIDSDGSTSYADSVKGRFTISKDNAKNTLYLQMNSLKPEDTAMYY | |
| | CAADPIPGPGYCDGGPNKYWGQGTQVTVSSGGGSQVQLQESGGGSVQ | |
| | AGGSLRLSCAVSGYAYSTYCMGWFRQAPGKEREGVAAIDSGGSTSYA | |
| | DSVKGRFTISKDNAKNTLYLRMNSLKPEDTAMYYCAAVPPPPDGGSC | |
| | LFLGPEIKVSKADFRYWGQGTQVTVSSASHHHHHH | |
| 183-165 | QVQLQESGGGSVQAGGSLRLSCAASGYTYSSYCMGWFRQAPGKEREG | 316 |
| | VAHIDSDGSTSYADSVKGRFTISKDNAKNTLYLQMNSLKPEDTAMYY | |
| | CAADPIPGPGYCDGGPNKYWGQGTQVTVSSGGGSQVQLQESGGGSVQ | |
| | AGGSLRLSCTVSGYTYSSNCMGWFRQAPGKEREGVATIYTGGGNTYY | |
| | ADSVKGRFTISQDNAKNTVYLQMNNLKPEDTAMYYCAAEPLSRVYGG | |
| | SCPTPTFDYWGQGTQVTVSSASHHHHHH | |
| 183-167 | QVQLQESGGGSVQAGGSLRLSCAASGYTYSSYCMGWFRQAPGKEREG | 317 |
| | VAHIDSDGSTSYADSVKGRFTISKDNAKNTLYLQMNSLKPEDTAMYY | |
| | CAADPIPGPGYCDGGPNKYWGQGTQVTVSSGGGSQVQLQESGGGSVQ | |
| | AGGSLRLSCGASGYTYSSYCMGWFRQVPGKEREGVAVIDSDGSTSYA | |
| | DSVKGRFTISKDNGKNTLYLQMNSLKPEDTAMYYCAADLGHYRPPCG | |
| | VLYLGMDYWGKGTQVTVSSASHHHHHH | |
| 183-170 | QVQLQESGGGSVQAGGSLRLSCAASGYTYSSYCMGWFRQAPGKEREG | 318 |
| | VAHIDSDGSTSYADSVKGRFTISKDNAKNTLYLQMNSLKPEDTAMYY | |
| | CAADPIPGPGYCDGGPNKYWGQGTQVTVSSGGGSQVQLQESGGGSVQ | |
| | AGGSLRLSCAASGYSNCSYDMTWYRQAPGKEREFVSAIHSDGSTRYA | |
| | DSVKGRFFISQDNAKNTVYLQMNSLKPEDTAMYYCKTDPLHCRAHGG | |
| | SWYSVRANYWGQGTQVTVSSASHHHHHH | |
| 174-163 | QVQLQESGGGSVQAGGSLRLSCAASRYTYNSYCMGWFRQAPGKEREG | 319 |
| | VATIDSDGMTRYADSVKGRFTISKDNAKNTLYLQMNSLKPEDTAMYY | |
| | CAADADCTIAAMTTNPLGQGTQVTVSSGGGSQVQLQESGGGSVQAGG | |
| | SLRLSCAASRYLYSIDYMAWFRQSPGKEREPVAVIYTASGATFYPDS | |
| | VKGRFTISQDNAKMTVYLQMNSLKSEDTAMYYCAAVRKTDSYLFDAQ | |
| | SFTYWGQGTQVTVSSASHHHHHH | |
| 174-159 | QVQLQESGGGSVQAGGSLRLSCAASRYTYNSYCMGWFRQAPGKEREG | 320 |
| | VATIDSDGMTRYADSVKGRFTISKDNAKNTLYLQMNSLKPEDTAMYY | |
| | CAADADCTIAAMTTNPLGQGTQVTVSSGGGSQVQLQESGGGSVQAGG | |
| | SLRLSCAASRFTYSSYCMGWFRQAPGKEREGVASIDSDGSTSYTDSV | |
| | KGRFTISKDNAKNTLYLQMNSLKPEDTAMYYCALDLMSTVVPGFCGF | |
| | LLSAGMDYWGKGTQVTVSSASHHHHHH | |
| 174-161 | QVQLQESGGGSVQAGGSLRLSCAASRYTYNSYCMGWFRQAPGKEREG | 321 |
| | VATIDSDGMTRYADSVKGRFTISKDNAKNTLYLQMNSLKPEDTAMYY | |
| | CAADADCTIAAMTTNPLGQGTQVTVSSGGGSQVQLQESGGGSVQAGG | |
| | SLRLSCAASGYTYSMYCMGWFRQAPGKEREGVAQINSDGSTSYADSV | |
| | KGRFTISKDNAKNTLYLQMNSLKPEDTAMYYCAADSRVYGGSWYERL | |
| | CGPYTYEYNYWGQGTQVTVSSASHHHHHH | |
| 174-162 | QVQLQESGGGSVQAGGSLRLSCAASRYTYNSYCMGWFRQAPGKEREG | 322 |
| | VATIDSDGMTRYADSVKGRFTISKDNAKNTLYLQMNSLKPEDTAMYY | |
| | CAADADCTIAAMTTNPLGQGTQVTVSSGGGSQVQLQESGGGSVQAGG | |
| | SLRLSCAVSGYAYSTYCMGWFRQAPGKEREGVAAIDSGGSTSYADSV | |
| | KGRFTISKDNAKNTLYLRMNSLKPEDTAMYYCAAVPPPPDGGSCLFL | |
| | GPEIKVSKADFRYWGQGTQVTVSSASHHHHHH | |
| 174-165 | QVQLQESGGGSVQAGGSLRLSCAASRYTYNSYCMGWFRQAPGKEREG | 323 |
| | VATIDSDGMTRYADSVKGRFTISKDNAKNTLYLQMNSLKPEDTAMYY | |
| | CAADADCTIAAMTTNPLGQGTQVTVSSGGGSQVQLQESGGGSVQAGG | |
| | SLRLSCTVSGYTYSSNCMGWFRQAPGKEREGVATIYTGGGNTYYADS | |
| | VKGRFTISQDNAKNTVYLQMNNLKPEDTAMYYCAAEPLSRVYGGSCP | |
| | TPTFDYWGQGTQVTVSSASHHHHHH | |
| 174-167 | QVQLQESGGGSVQAGGSLRLSCAASRYTYNSYCMGWFRQAPGKEREG | 324 |
| | VATIDSDGMTRYADSVKGRFTISKDNAKNTLYLQMNSLKPEDTAMYY | |
| | CAADADCTIAAMTTNPLGQGTQVTVSSGGGSQVQLQESGGGSVQAGG | |
| | SLRLSCGASGYTYSSYCMGWFRQVPGKEREGVAVIDSDGSTSYADSV | |
| | KGRFTISKDNGKNTLYLQMNSLKPEDTAMYYCAADLGHYRPPCGVLY | |
| | LGMDYWGKGTQVTVSSASHHHHHH | |
| 174-170 | QVQLQESGGGSVQAGGSLRLSCAASRYTYNSYCMGWFRQAPGKEREG | 325 |
| | VATIDSDGMTRYADSVKGRFTISKDNAKNTLYLQMNSLKPEDTAMYY | |
| | CAADADCTIAAMTTNPLGQGTQVTVSSGGGSQVQLQESGGGSVQAGG | |
| | SLRLSCAASGYSNCSYDMTWYRQAPGKEREFVSAIHSDGSTRYADSV | |
| | KGRFFISQDNAKNTVYLQMNSLKPEDTAMYYCKTDPLHCRAHGGSWY | |
| | SVRANYWGQGTQVTVSSASHHHHHH | |
| 186-163 | QVQLQESGGGSVQAGGSLRLSCTVSRYTASVNYMGWFRQAPGKEREG | 326 |
| | VATIFTGAGTTYYANSVKGRFTISRDNAKNTAYLQMNSLKPEDTAIY | |
| | YCAVDFRGGLLYRPAYEYTYRGQGTQVTVSSGGGSQVQLQESGGGSV | |
| | QAGGSLRLSCAASRYLYSIDYMAWFRQSPGKEREPVAVIYTASGATF | |
| | YPDSVKGRFTISQDNAKMTVYLQMNSLKSEDTAMYYCAAVRKTDSYL | |
| | FDAQSFTYWGQGTQVTVSSASHHHHHH | |
| 186-159 | QVQLQESGGGSVQAGGSLRLSCTVSRYTASVNYMGWFRQAPGKEREG | 327 |
| | VATIFTGAGTTYYANSVKGRFTISRDNAKNTAYLQMNSLKPEDTAIY | |
| | YCAVDFRGGLLYRPAYEYTYRGQGTQVTVSSGGGSQVQLQESGGGSV | |
| | QAGGSLRLSCAASRFTYSSYCMGWFRQAPGKEREGVASIDSDGSTSY | |
| | TDSVKGRFTISKDNAKNTLYLQMNSLKPEDTAMYYCALDLMSTVVPG | |
| | FCGFLLSAGMDYWGKGTQVTVSSASHHHHHH | |
| 186-161 | QVQLQESGGGSVQAGGSLRLSCTVSRYTASVNYMGWFRQAPGKEREG | 328 |
| | VATIFTGAGTTYYANSVKGRFTISRDNAKNTAYLQMNSLKPEDTAIY | |
| | YCAVDFRGGLLYRPAYEYTYRGQGTQVTVSSGGGSQVQLQESGGGSV | |
| | QAGGSLRLSCAASGYTYSMYCMGWFRQAPGKEREGVAQINSDGSTSY | |
| | ADSVKGRFTISKDNAKNTLYLQMNSLKPEDTAMYYCAADSRVYGGSW | |
| | YERLCGPYTYEYNYWGQGTQVTVSSASHHHHHH | |
| 186-162 | QVQLQESGGGSVQAGGSLRLSCTVSRYTASVNYMGWFRQAPGKEREG | 329 |
| | VATIFTGAGTTYYANSVKGRFTISRDNAKNTAYLQMNSLKPEDTAIY | |
| | YCAVDFRGGLLYRPAYEYTYRGQGTQVTVSSGGGSQVQLQESGGGSV | |
| | QAGGSLRLSCAVSGYAYSTYCMGWFRQAPGKEREGVAAIDSGGSTSY | |
| | ADSVKGRFTISKDNAKNTLYLRMNSLKPEDTAMYYCAAVPPPPDGGS | |
| | CLFLGPEIKVSKADFRYWGQGTQVTVSSASHHHHHH | |
| 186-165 | QVQLQESGGGSVQAGGSLRLSCTVSRYTASVNYMGWFRQAPGKEREG | 330 |
| | VATIFTGAGTTYYANSVKGRFTISRDNAKNTAYLQMNSLKPEDTAIY | |
| | YCAVDFRGGLLYRPAYEYTYRGQGTQVTVSSGGGSQVQLQESGGGSV | |
| | QAGGSLRLSCTVSGYTYSSNCMGWFRQAPGKEREGVATIYTGGGNTY | |
| | YADSVKGRFTISQDNAKNTVYLQMNNLKPEDTAMYYCAAEPLSRVYG | |
| | GSCPTPTFDYWGQGTQVTVSSASHHHHHH | |
| 186-167 | QVQLQESGGGSVQAGGSLRLSCTVSRYTASVNYMGWFRQAPGKEREG | 331 |
| | VATIFTGAGTTYYANSVKGRFTISRDNAKNTAYLQMNSLKPEDTAIY | |
| | YCAVDFRGGLLYRPAYEYTYRGQGTQVTVSSGGGSQVQLQESGGGSV | |
| | QAGGSLRLSCGASGYTYSSYCMGWFRQVPGKEREGVAVIDSDGSTSY | |
| | ADSVKGRFTISKDNGKNTLYLQMNSLKPEDTAMYYCAADLGHYRPPC | |
| | GVLYLGMDYWGKGTQVTVSSASHHHHHH | |
| 186-170 | QVQLQESGGGSVQAGGSLRLSCTVSRYTASVNYMGWFRQAPGKEREG | 332 |
| | VATIFTGAGTTYYANSVKGRFTISRDNAKNTAYLQMNSLKPEDTAIY | |
| | YCAVDFRGGLLYRPAYEYTYRGQGTQVTVSSGGGSQVQLQESGGGSV | |
| | QAGGSLRLSCAASGYSNCSYDMTWYRQAPGKEREFVSAIHSDGSTRY | |
| | ADSVKGRFFISQDNAKNTVYLQMNSLKPEDTAMYYCKTDPLHCRAHG | |
| | GSWYSVRANYWGQGTQVTVSSASHHHHHH | |
| 187-163 | QVQLQESGGGSVEAGGSLRLSCAASGYTHSSYCMGWFRQAPGKEREG | 333 |
| | VAAIDVDGSTTYADSVKGRFTISKDNAKNTLYLQMNSLKPEDTGMYY | |
| | CAAEFADCSSNYFLPPGAVRYWGQGTQVTVSSGGGSQVQLQESGGGS | |
| | VQAGGSLRLSCAASRYLYSIDYMAWFRQSPGKEREPVAVIYTASGAT | |
| | FYPDSVKGRFTISQDNAKMTVYLQMNSLKSEDTAMYYCAAVRKTDSY | |
| | LFDAQSFTYWGQGTQVTVSSASHHHHHH | |
| 187-159 | QVQLQESGGGSVEAGGSLRLSCAASGYTHSSYCMGWFRQAPGKEREG | 334 |
| | VAAIDVDGSTTYADSVKGRFTISKDNAKNTLYLQMNSLKPEDTGMYY | |
| | CAAEFADCSSNYFLPPGAVRYWGQGTQVTVSSGGGSQVQLQESGGGS | |
| | VQAGGSLRLSCAASRFTYSSYCMGWFRQAPGKEREGVASIDSDGSTS | |
| | YTDSVKGRFTISKDNAKNTLYLQMNSLKPEDTAMYYCALDLMSTVVP | |
| | GFCGFLLSAGMDYWGKGTQVTVSSASHHHHHH | |
| 187-161 | QVQLQESGGGSVEAGGSLRLSCAASGYTHSSYCMGWFRQAPGKEREG | 335 |
| | VAAIDVDGSTTYADSVKGRFTISKDNAKNTLYLQMNSLKPEDTGMYY | |
| | CAAEFADCSSNYFLPPGAVRYWGQGTQVTVSSGGGSQVQLQESGGGS | |
| | VQAGGSLRLSCAASGYTYSMYCMGWFRQAPGKEREGVAQINSDGSTS | |
| | YADSVKGRFTISKDNAKNTLYLQMNSLKPEDTAMYYCAADSRVYGGS | |
| | WYERLCGPYTYEYNYWGQGTQVTVSSASHHHHHH | |
| 187-162 | QVQLQESGGGSVEAGGSLRLSCAASGYTHSSYCMGWFRQAPGKEREG | 336 |
| | VAAIDVDGSTTYADSVKGRFTISKDNAKNTLYLQMNSLKPEDTGMYY | |
| | CAAEFADCSSNYFLPPGAVRYWGQGTQVTVSSGGGSQVQLQESGGGS | |
| | VQAGGSLRLSCAVSGYAYSTYCMGWFRQAPGKEREGVAAIDSGGSTS | |
| | YADSVKGRFTISKDNAKNTLYLRMNSLKPEDTAMYYCAAVPPPPDGG | |
| | SCLFLGPEIKVSKADFRYWGQGTQVTVSSASHHHHHH | |
| 187-165 | QVQLQESGGGSVEAGGSLRLSCAASGYTHSSYCMGWFRQAPGKEREG | 337 |
| | VAAIDVDGSTTYADSVKGRFTISKDNAKNTLYLQMNSLKPEDTGMYY | |
| | CAAEFADCSSNYFLPPGAVRYWGQGTQVTVSSGGGSQVQLQESGGGS | |
| | VQAGGSLRLSCTVSGYTYSSNCMGWFRQAPGKEREGVATIYTGGGNT | |
| | YYADSVKGRFTISQDNAKNTVYLQMNNLKPEDTAMYYCAAEPLSRVY | |
| | GGSCPTPTFDYWGQGTQVTVSSASHHHHHH | |
| 187-167 | QVQLQESGGGSVEAGGSLRLSCAASGYTHSSYCMGWFRQAPGKEREG | 338 |
| | VAAIDVDGSTTYADSVKGRFTISKDNAKNTLYLQMNSLKPEDTGMYY | |
| | CAAEFADCSSNYFLPPGAVRYWGQGTQVTVSSGGGSQVQLQESGGGS | |
| | VQAGGSLRLSCGASGYTYSSYCMGWFRQVPGKEREGVAVIDSDGSTS | |
| | YADSVKGRFTISKDNGKNTLYLQMNSLKPEDTAMYYCAADLGHYRPP | |
| | CGVLYLGMDYWGKGTQVTVSSASHHHHHH | |
| 187-170 | QVQLQESGGGSVEAGGSLRLSCAASGYTHSSYCMGWFRQAPGKEREG | 339 |
| | VAAIDVDGSTTYADSVKGRFTISKDNAKNTLYLQMNSLKPEDTGMYY | |
| | CAAEFADCSSNYFLPPGAVRYWGQGTQVTVSSGGGSQVQLQESGGGS | |
| | VQAGGSLRLSCAASGYSNCSYDMTWYRQAPGKEREFVSAIHSDGSTR | |
| | YADSVKGRFFISQDNAKNTVYLQMNSLKPEDTAMYYCKTDPLHCRAH | |
| | GGSWYSVRANYWGQGTQVTVSSASHHHHHH | |
| 197-163 | QVQLQESGGGSVQAGGSLRLSCAASGYSYSSYCMGWFRQAPGKEREG | 340 |
| | VATIDSDGMTRYADSVKGRFTISKDNAKNTLYLQMNSLKPEDTAMYY | |
| | CAAPLYDCDSGAVGRNPPYWGQGTQVTVSSGGGSQVQLQESGGGSVQ | |
| | AGGSLRLSCAASRYLYSIDYMAWFRQSPGKEREPVAVIYTASGATFY | |
| | PDSVKGRFTISQDNAKMTVYLQMNSLKSEDTAMYYCAAVRKTDSYLF | |
| | DAQSFTYWGQGTQVTVSSASHHHHHH | |
| 197-159 | QVQLQESGGGSVQAGGSLRLSCAASGYSYSSYCMGWFRQAPGKEREG | 341 |
| | VATIDSDGMTRYADSVKGRFTISKDNAKNTLYLQMNSLKPEDTAMYY | |
| | CAAPLYDCDSGAVGRNPPYWGQGTQVTVSSGGGSQVQLQESGGGSVQ | |
| | AGGSLRLSCAASRFTYSSYCMGWFRQAPGKEREGVASIDSDGSTSYT | |
| | DSVKGRFTISKDNAKNTLYLQMNSLKPEDTAMYYCALDLMSTVVPGF | |
| | CGFLLSAGMDYWGKGTQVTVSSASHHHHHH | |
| 197-161 | QVQLQESGGGSVQAGGSLRLSCAASGYSYSSYCMGWFRQAPGKEREG | 342 |
| | VATIDSDGMTRYADSVKGRFTISKDNAKNTLYLQMNSLKPEDTAMYY | |
| | CAAPLYDCDSGAVGRNPPYWGQGTQVTVSSGGGSQVQLQESGGGSVQ | |
| | AGGSLRLSCAASGYTYSMYCMGWFRQAPGKEREGVAQINSDGSTSYA | |
| | DSVKGRFTISKDNAKNTLYLQMNSLKPEDTAMYYCAADSRVYGGSWY | |
| | ERLCGPYTYEYNYWGQGTQVTVSSASHHHHHH | |
| 197-162 | QVQLQESGGGSVQAGGSLRLSCAASGYSYSSYCMGWFRQAPGKEREG | 343 |
| | VATIDSDGMTRYADSVKGRFTISKDNAKNTLYLQMNSLKPEDTAMYY | |
| | CAAPLYDCDSGAVGRNPPYWGQGTQVTVSSGGGSQVQLQESGGGSVQ | |
| | AGGSLRLSCAVSGYAYSTYCMGWFRQAPGKEREGVAAIDSGGSTSYA | |
| | DSVKGRFTISKDNAKNTLYLRMNSLKPEDTAMYYCAAVPPPPDGGSC | |
| | LFLGPEIKVSKADFRYWGQGTQVTVSSASHHHHHH | |
| 197-165 | QVQLQESGGGSVQAGGSLRLSCAASGYSYSSYCMGWFRQAPGKEREG | 344 |
| | VATIDSDGMTRYADSVKGRFTISKDNAKNTLYLQMNSLKPEDTAMYY | |
| | CAAPLYDCDSGAVGRNPPYWGQGTQVTVSSGGGSQVQLQESGGGSVQ | |
| | AGGSLRLSCTVSGYTYSSNCMGWFRQAPGKEREGVATIYTGGGNTYY | |
| | ADSVKGRFTISQDNAKNTVYLQMNNLKPEDTAMYYCAAEPLSRVYGG | |
| | SCPTPTFDYWGQGTQVTVSSASHHHHHH | |
| 197-167 | QVQLQESGGGSVQAGGSLRLSCAASGYSYSSYCMGWFRQAPGKEREG | 345 |
| | VATIDSDGMTRYADSVKGRFTISKDNAKNTLYLQMNSLKPEDTAMYY | |
| | CAAPLYDCDSGAVGRNPPYWGQGTQVTVSSGGGSQVQLQESGGGSVQ | |
| | AGGSLRLSCGASGYTYSSYCMGWFRQVPGKEREGVAVIDSDGSTSYA | |
| | DSVKGRFTISKDNGKNTLYLQMNSLKPEDTAMYYCAADLGHYRPPCG | |
| | VLYLGMDYWGKGTQVTVSSASHHHHHH | |
| 197-170 | QVQLQESGGGSVQAGGSLRLSCAASGYSYSSYCMGWFRQAPGKEREG | 346 |
| | VATIDSDGMTRYADSVKGRFTISKDNAKNTLYLQMNSLKPEDTAMYY | |
| | CAAPLYDCDSGAVGRNPPYWGQGTQVTVSSGGGSQVQLQESGGGSVQ | |
| | AGGSLRLSCAASGYSNCSYDMTWYRQAPGKEREFVSAIHSDGSTRYA | |
| | DSVKGRFFISQDNAKNTVYLQMNSLKPEDTAMYYCKTDPLHCRAHGG | |
| | SWYSVRANYWGQGTQVTVSSASHHHHHH | |
| 198-163 | QVQLQESGGGSVQTGGSLRLSCAASGYTYLRGCMGWFRQAPGKEREG | 347 |
| | VAVMDVVGDRRSYIDSVKGRFTISRDNAANSVYLQMDNLKPEDTAMY | |
| | YCTAGPNCVGWRSGLDYWGQGTQVTVSSGGGSQVQLQESGGGSVQAG | |
| | GSLRLSCAASRYLYSIDYMAWFRQSPGKEREPVAVIYTASGATFYPD | |
| | SVKGRFTISQDNAKMTVYLQMNSLKSEDTAMYYCAAVRKTDSYLFDA | |
| | QSFTYWGQGTQVTVSSASHHHHHH | |
| 198-159 | QVQLQESGGGSVQTGGSLRLSCAASGYTYLRGCMGWFRQAPGKEREG | 348 |
| | VAVMDVVGDRRSYIDSVKGRFTISRDNAANSVYLQMDNLKPEDTAMY | |
| | YCTAGPNCVGWRSGLDYWGQGTQVTVSSGGGSQVQLQESGGGSVQAG | |
| | GSLRLSCAASRFTYSSYCMGWFRQAPGKEREGVASIDSDGSTSYTDS | |
| | VKGRFTISKDNAKNTLYLQMNSLKPEDTAMYYCALDLMSTVVPGFCG | |
| | FLLSAGMDYWGKGTQVTVSSASHHHHHH | |
| 198-161 | QVQLQESGGGSVQTGGSLRLSCAASGYTYLRGCMGWFRQAPGKEREG | 349 |
| | VAVMDVVGDRRSYIDSVKGRFTISRDNAANSVYLQMDNLKPEDTAMY | |
| | YCTAGPNCVGWRSGLDYWGQGTQVTVSSGGGSQVQLQESGGGSVQAG | |
| | GSLRLSCAASGYTYSMYCMGWFRQAPGKEREGVAQINSDGSTSYADS | |
| | VKGRFTISKDNAKNTLYLQMNSLKPEDTAMYYCAADSRVYGGSWYER | |
| | LCGPYTYEYNYWGQGTQVTVSSASHHHHHH | |
| 198-162 | QVQLQESGGGSVQTGGSLRLSCAASGYTYLRGCMGWFRQAPGKEREG | 350 |
| | VAVMDVVGDRRSYIDSVKGRFTISRDNAANSVYLQMDNLKPEDTAMY | |
| | YCTAGPNCVGWRSGLDYWGQGTQVTVSSGGGSQVQLQESGGGSVQAG | |
| | GSLRLSCAVSGYAYSTYCMGWFRQAPGKEREGVAAIDSGGSTSYADS | |
| | VKGRFTISKDNAKNTLYLRMNSLKPEDTAMYYCAAVPPPPDGGSCLF | |
| | LGPEIKVSKADFRYWGQGTQVTVSSASHHHHHH | |
| 198-165 | QVQLQESGGGSVQTGGSLRLSCAASGYTYLRGCMGWFRQAPGKEREG | 351 |
| | VAVMDVVGDRRSYIDSVKGRFTISRDNAANSVYLQMDNLKPEDTAMY | |
| | YCTAGPNCVGWRSGLDYWGQGTQVTVSSGGGSQVQLQESGGGSVQAG | |
| | GSLRLSCTVSGYTYSSNCMGWFRQAPGKEREGVATIYTGGGNTYYAD | |
| | SVKGRFTISQDNAKNTVYLQMNNLKPEDTAMYYCAAEPLSRVYGGSC | |
| | PTPTFDYWGQGTQVTVSSASHHHHHH | |
| 198-167 | QVQLQESGGGSVQTGGSLRLSCAASGYTYLRGCMGWFRQAPGKEREG | 352 |
| | VAVMDVVGDRRSYIDSVKGRFTISRDNAANSVYLQMDNLKPEDTAMY | |
| | YCTAGPNCVGWRSGLDYWGQGTQVTVSSGGGSQVQLQESGGGSVQAG | |
| | GSLRLSCGASGYTYSSYCMGWFRQVPGKEREGVAVIDSDGSTSYADS | |
| | VKGRFTISKDNGKNTLYLQMNSLKPEDTAMYYCAADLGHYRPPCGVL | |
| | YLGMDYWGKGTQVTVSSASHHHHHH | |
| 198-170 | QVQLQESGGGSVQTGGSLRLSCAASGYTYLRGCMGWFRQAPGKEREG | 353 |
| | VAVMDVVGDRRSYIDSVKGRFTISRDNAANSVYLQMDNLKPEDTAMY | |
| | YCTAGPNCVGWRSGLDYWGQGTQVTVSSGGGSQVQLQESGGGSVQAG | |
| | GSLRLSCAASGYSNCSYDMTWYRQAPGKEREFVSAIHSDGSTRYADS | |
| | VKGRFFISQDNAKNTVYLQMNSLKPEDTAMYYCKTDPLHCRAHGGSW | |
| | YSVRANYWGQGTQVTVSSASHHHHHH | |

TABLE 13
IL10R Binding Protein Activity

| TestArticle | Abs 630 | Abs 630 |
|---|---|---|
| (SEQ ID NO:) | (25 nM) | (100 nM) |
| Unstimulated | 0.58 | 0.59 |
| WildType hIL10 | 2.08 | 2.04 |
| 256 | 0.49 | 0.39 |
| 257 | 0.45 | 0.36 |
| 258 | 0.49 | 0.38 |
| 259 | 1.85 | 1.13 |
| 260 | 0.49 | 0.39 |
| 261 | 0.44 | 0.34 |
| 262 | 1.38 | 0.40 |
| 263 | 1.77 | 1.02 |
| 264 | 1.52 | 0.67 |
| 265 | 0.54 | 0.46 |
| 266 | 0.49 | 0.39 |
| 267 | 0.75 | 0.53 |
| 268 | 0.53 | 0.41 |
| 269 | 0.46 | 0.37 |
| 270 | 1.41 | 0.73 |
| 271 | 1.93 | 1.65 |
| 272 | 0.47 | 0.38 |
| 273 | 0.52 | 0.41 |
| 274 | 0.46 | 0.37 |
| 275 | 0.51 | 0.36 |
| 276 | 0.46 | 0.36 |
| 277 | 1.19 | 1.00 |
| 278 | 1.61 | 1.18 |
| 279 | 0.49 | 0.39 |
| 280 | 0.49 | 0.37 |
| 281 | 0.66 | 0.69 |
| 282 | 0.44 | 0.36 |
| 283 | 0.48 | 0.39 |
| 284 | 0.45 | 0.34 |
| 285 | 0.48 | 0.39 |
| 286 | 0.46 | 0.39 |
| 287 | 0.90 | 0.51 |
| 288 | 0.50 | 0.44 |
| 289 | 0.48 | 0.39 |
| 290 | 0.49 | 0.37 |
| 291 | 1.73 | 0.59 |
| 292 | 0.78 | 0.47 |
| 293 | 0.54 | 0.43 |
| 294 | 0.49 | 0.39 |
| 295 | 0.72 | 0.46 |
| 296 | 0.54 | 0.38 |
| 297 | 0.46 | 0.36 |
| 298 | 0.84 | 0.38 |
| 299 | 0.47 | 0.37 |
| 300 | 2.08 | 2.11 |
| 301 | 2.05 | 1.91 |
| 302 | 1.98 | 2.09 |
| 303 | 1.92 | 1.93 |
| 304 | 1.96 | 2.06 |
| 305 | 0.59 | 0.35 |
| 306 | 0.69 | 0.49 |
| 307 | 0.44 | 0.34 |
| 308 | 0.48 | 0.39 |
| 309 | 0.45 | 0.37 |
| 310 | 0.51 | 0.44 |
| 311 | 0.48 | 0.40 |
| 312 | 0.48 | 0.39 |
| 313 | 0.47 | 0.39 |
| 314 | 0.49 | 0.42 |
| 315 | 0.51 | 0.41 |
| 316 | 0.48 | 0.39 |
| 317 | 0.45 | 0.38 |
| 318 | 0.50 | 0.45 |
| 319 | 0.47 | 0.36 |
| 320 | 0.54 | 0.41 |
| 321 | 0.46 | 0.40 |
| 322 | 0.46 | 0.41 |
| 323 | 0.64 | 0.38 |
| 324 | 0.61 | 0.44 |
| 325 | 0.49 | 0.42 |
| 326 | 0.47 | 0.56 |
| 327 | 0.52 | 0.54 |
| 328 | 0.44 | 0.34 |
| 329 | 0.48 | 0.39 |
| 330 | 0.45 | 0.36 |
| 331 | 0.50 | 0.41 |
| 332 | 0.47 | 0.36 |
| 333 | 0.49 | 0.54 |
| 334 | 1.43 | 1.14 |
| 335 | 0.50 | 0.44 |
| 336 | 0.54 | 0.45 |
| 337 | 0.49 | 0.40 |
| 338 | 0.51 | 0.41 |
| 339 | 0.49 | 0.42 |
| 340 | 0.46 | 0.51 |
| 341 | 0.60 | 0.48 |
| 342 | 0.45 | 0.36 |
| 343 | 0.46 | 0.37 |
| 344 | 0.50 | 0.43 |
| 345 | 0.52 | 0.44 |
| 346 | 0.43 | 0.35 |
| 347 | 0.45 | 0.35 |
| 348 | 0.46 | 0.36 |
| 349 | 0.46 | 0.38 |
| 350 | 0.43 | 0.35 |
| 351 | 0.43 | 0.34 |
| 352 | 0.54 | 0.65 |
| 353 | 0.56 | 0.61 |

TABLE 14
IL10 Receptor Binding Molecules Nucleic Acids

| DNA | | |
|---|---|---|
| ENCODING | | |
| SEQ ID | | SEQ ID |
| NO. | DNA Sequence | NO |
| 256 | CAAGTGCAGCTGCAAGAGAGCGGAGGAGGAAGCGTGCAAGCCGGAGGC | 354 |
| | TCTCTGAGACTGAGCTGTGCCGCCTCTAGGTATCTGTACAGCATCGAC | |
| | TACATGGCTTGGTTCAGACAGAGCCCCGGCAAGGAGAGGGAGCCAGTG | |
| | GCTGTCATCTACACTGCCTCCGGCGCCACATTCTATCCAGATAGCGTG | |
| | AAGGGAAGGTTCACTATCAGCCAAGATAACGCCAAGATGACAGTGTAT | |
| | CTGCAGATGAACTCTCTGAAGAGCGAGGACACTGCCATGTACTACTGT | |
| | GCCGCCGTGAGGAAGACAGATAGCTACCTCTTCGACGCCCAGAGCTTC | |
| | ACATACTGGGGCCAAGGCACACAAGTGACAGTCTCGAGCGGCGGAGGA | |
| | TCCCAAGTGCAGCTGCAAGAGAGCGGCGGAGGAAGCGTGCAAGCTGGA | |
| | GGCTCTCTGAGGCTGAGCTGTGCTGCCAGCGGCTACACTTATAGCAGC | |
| | GGCTGTATGGGCTGGTTCAGACAAGCCCCCGGCAAGGAAAGGGAAGCC | |
| | GTGGCCGCCATCAATTCCGATGGCAGCACAAGCTACGCCGACAGCGTG | |
| | AAGGGAAGGTTCACAATCAGCAAGGACAACGCCAAGAACACACTCTAT | |
| | CTGCAGATGAACTCTCTGAAGCCAGAGGACACAGCCATGTACTACTGC | |
| | GCCGCTGAGCCTTACTGTAGCGGCGGCTACCCAAGATGGAGCGTCGCT | |
| | GAGTTCGGCTACTGGGGCCAAGGCACACAAGTGACTGTCTCGTCTGCT | |
| | AGCCACCATCACCATCACCAC | |
| 257 | CAAGTGCAGCTGCAAGAGAGCGGAGGAGGAAGCGTGCAAGCCGGAGGC | 355 |
| | TCTCTGAGACTGAGCTGTGCCGCCTCTAGGTATCTGTACAGCATCGAC | |
| | TACATGGCTTGGTTCAGACAGAGCCCCGGCAAGGAGAGGGAGCCAGTG | |
| | GCTGTCATCTACACTGCCTCCGGCGCCACATTCTATCCAGATAGCGTG | |
| | AAGGGAAGGTTCACTATCAGCCAAGATAACGCCAAGATGACAGTGTAT | |
| | CTGCAGATGAACTCTCTGAAGAGCGAGGACACTGCCATGTACTACTGT | |
| | GCCGCCGTGAGGAAGACAGATAGCTACCTCTTCGACGCCCAGAGCTTC | |
| | ACATACTGGGGCCAAGGCACACAAGTGACAGTCTCGAGCGGCGGAGGA | |
| | TCCCAAGTGCAGCTGCAAGAGAGCGGAGGAGGCAGCGTCCAAGCCGGA | |
| | GGCTCTCTGAGGCTGAGCTGTGCTGCCAGCGGCTACACTTACAGCAGC | |
| | TACTGCATGGGCTGGTTCAGACAAGCCCCCGGCAAGGAGAGAGAGGGC | |
| | GTGGCTCACATCGACAGCGACGGCTCCACAAGCTACGCCGATAGCGTG | |
| | AAGGGAAGGTTCACAATCTCCAAGGACAACGCCAAGAACACTCTGTAC | |
| | CTCCAGATGAACTCTCTGAAGCCAGAGGACACTGCCATGTACTACTGT | |
| | GCCGCCGATCCAATTCCCGGCCCCGGCTACTGCGATGGCGGCCCTAAC | |
| | AAGTACTGGGGCCAAGGCACACAAGTGACTGTCTCGTCTGCTAGCCAC | |
| | CATCACCATCACCAC | |
| 258 | CAAGTGCAGCTGCAAGAGAGCGGAGGAGGAAGCGTGCAAGCCGGAGGC | 356 |
| | TCTCTGAGACTGAGCTGTGCCGCCTCTAGGTATCTGTACAGCATCGAC | |
| | TACATGGCTTGGTTCAGACAGAGCCCCGGCAAGGAGAGGGAGCCAGTG | |
| | GCTGTCATCTACACTGCCTCCGGCGCCACATTCTATCCAGATAGCGTG | |
| | AAGGGAAGGTTCACTATCAGCCAAGATAACGCCAAGATGACAGTGTAT | |
| | CTGCAGATGAACTCTCTGAAGAGCGAGGACACTGCCATGTACTACTGT | |
| | GCCGCCGTGAGGAAGACAGATAGCTACCTCTTCGACGCCCAGAGCTTC | |
| | ACATACTGGGGCCAAGGCACACAAGTGACAGTCTCGAGCGGCGGAGGA | |
| | TCCCAAGTGCAGCTGCAAGAGTCCGGAGGAGGAAGCGTGCAAGCCGGC | |
| | GGATCTCTGAGACTGAGCTGTGCCGCCTCTAGGTACACTTACAACAGC | |
| | TACTGCATGGGCTGGTTCAGACAAGCCCCCGGCAAGGAAAGAGAGGGC | |
| | GTGGCCACTATCGATAGCGACGGCATGACTAGGTACGCTGATAGCGTC | |
| | AAGGGAAGGTTCACAATCTCCAAGGACAATGCTAAGAACACTCTGTAC | |
| | CTCCAGATGAACTCTCTGAAGCCAGAGGACACAGCCATGTACTACTGC | |
| | GCTGCCGATGCCGACTGCACTATCGCCGCCATGACTACTAATCCTCTG | |
| | GGCCAAGGCACACAAGTGACTGTCTCGTCTGCTAGCCACCATCACCAT | |
| | CACCAC | |
| 259 | CAAGTGCAGCTGCAAGAGAGCGGAGGAGGAAGCGTGCAAGCCGGAGGC | 357 |
| | TCTCTGAGACTGAGCTGTGCCGCCTCTAGGTATCTGTACAGCATCGAC | |
| | TACATGGCTTGGTTCAGACAGAGCCCCGGCAAGGAGAGGGAGCCAGTG | |
| | GCTGTCATCTACACTGCCTCCGGCGCCACATTCTATCCAGATAGCGTG | |
| | AAGGGAAGGTTCACTATCAGCCAAGATAACGCCAAGATGACAGTGTAT | |
| | CTGCAGATGAACTCTCTGAAGAGCGAGGACACTGCCATGTACTACTGT | |
| | GCCGCCGTGAGGAAGACAGATAGCTACCTCTTCGACGCCCAGAGCTTC | |
| | ACATACTGGGGCCAAGGCACACAAGTGACAGTCTCGAGCGGCGGAGGA | |
| | TCCCAAGTGCAGCTGCAAGAGTCCGGCGGAGGCAGCGTCCAAGCCGGA | |
| | GGATCTCTGAGGCTGAGCTGTACAGTGAGCAGATACACTGCCAGCGTG | |
| | AACTACATGGGCTGGTTCAGACAAGCCCCCGGCAAAGAGAGAGAGGGC | |
| | GTGGCCACAATCTTCACTGGCGCCGGCACAACATACTACGCCAACTCC | |
| | GTCAAGGGAAGGTTCACAATCTCTAGGGACAACGCCAAGAACACTGCC | |
| | TATCTGCAGATGAACTCCCTCAAGCCAGAGGACACTGCCATCTACTAC | |
| | TGCGCCGTGGATTTCAGAGGCGGACTGCTGTATAGGCCAGCCTACGAG | |
| | TACACTTATAGGGGCCAAGGCACACAAGTGACAGTCTCGTCTGCTAGC | |
| | CACCATCACCATCACCAC | |
| 260 | CAAGTGCAGCTGCAAGAGAGCGGAGGAGGAAGCGTGCAAGCCGGAGGC | 358 |
| | TCTCTGAGACTGAGCTGTGCCGCCTCTAGGTATCTGTACAGCATCGAC | |
| | TACATGGCTTGGTTCAGACAGAGCCCCGGCAAGGAGAGGGAGCCAGTG | |
| | GCTGTCATCTACACTGCCTCCGGCGCCACATTCTATCCAGATAGCGTG | |
| | AAGGGAAGGTTCACTATCAGCCAAGATAACGCCAAGATGACAGTGTAT | |
| | CTGCAGATGAACTCTCTGAAGAGCGAGGACACTGCCATGTACTACTGT | |
| | GCCGCCGTGAGGAAGACAGATAGCTACCTCTTCGACGCCCAGAGCTTC | |
| | ACATACTGGGGCCAAGGCACACAAGTGACAGTCTCGAGCGGCGGAGGA | |
| | TCCCAAGTGCAGCTGCAAGAGAGCGGAGGAGGCAGCGTCGAAGCTGGA | |
| | GGATCTCTGAGGCTGAGCTGTGCTGCCAGCGGCTACACTCACAGCAGC | |
| | TACTGTATGGGCTGGTTCAGACAAGCCCCCGGCAAGGAGAGGGAAGGC | |
| | GTGGCTGCCATCGACGTGGATGGCAGCACTACTTACGCCGACAGCGTG | |
| | AAGGGAAGGTTCACTATCAGCAAGGACAACGCCAAGAACACACTCTAT | |
| | CTGCAGATGAACAGCCTCAAGCCAGAGGACACTGGCATGTACTACTGC | |
| | GCCGCCGAGTTCGCCGATTGCAGCAGCAACTACTTTCTGCCTCCCGGC | |
| | GCCGTCAGATATTGGGGCCAAGGCACTCAAGTGACAGTCTCGTCTGCT | |
| | AGCCACCATCACCATCACCAC | |
| 261 | CAAGTGCAGCTGCAAGAGAGCGGAGGAGGAAGCGTGCAAGCCGGAGGC | 359 |
| | TCTCTGAGACTGAGCTGTGCCGCCTCTAGGTATCTGTACAGCATCGAC | |
| | TACATGGCTTGGTTCAGACAGAGCCCCGGCAAGGAGAGGGAGCCAGTG | |
| | GCTGTCATCTACACTGCCTCCGGCGCCACATTCTATCCAGATAGCGTG | |
| | AAGGGAAGGTTCACTATCAGCCAAGATAACGCCAAGATGACAGTGTAT | |
| | CTGCAGATGAACTCTCTGAAGAGCGAGGACACTGCCATGTACTACTGT | |
| | GCCGCCGTGAGGAAGACAGATAGCTACCTCTTCGACGCCCAGAGCTTC | |
| | ACATACTGGGGCCAAGGCACACAAGTGACAGTCTCGAGCGGCGGAGGA | |
| | TCCCAAGTGCAGCTGCAAGAGAGCGGAGGAGGAAGCGTCCAAGCCGGA | |
| | GGATCTCTGAGACTGAGCTGCGCCGCTAGTGGCTACTCCTACAGCAGC | |
| | TACTGCATGGGCTGGTTTAGGCAAGCCCCCGGCAAGGAGAGAGAAGGC | |
| | GTGGCCACTATCGACAGCGACGGCATGACAAGGTACGCCGACAGCGTG | |
| | AAGGGAAGGTTCACAATCAGCAAGGACAACGCCAAGAACACACTGTAT | |
| | CTGCAGATGAACTCTCTGAAGCCAGAGGACACTGCCATGTACTACTGT | |
| | GCCGCTCCTCTGTACGACTGTGATAGCGGCGCTGTGGGCAGAAATCCA | |
| | CCTTATTGGGGCCAAGGCACTCAAGTGACAGTCTCGTCTGCTAGCCAC | |
| | CATCACCATCACCAC | |
| 262 | CAAGTGCAGCTGCAAGAGAGCGGAGGAGGAAGCGTGCAAGCCGGAGGC | 360 |
| | TCTCTGAGACTGAGCTGTGCCGCCTCTAGGTATCTGTACAGCATCGAC | |
| | TACATGGCTTGGTTCAGACAGAGCCCCGGCAAGGAGAGGGAGCCAGTG | |
| | GCTGTCATCTACACTGCCTCCGGCGCCACATTCTATCCAGATAGCGTG | |
| | AAGGGAAGGTTCACTATCAGCCAAGATAACGCCAAGATGACAGTGTAT | |
| | CTGCAGATGAACTCTCTGAAGAGCGAGGACACTGCCATGTACTACTGT | |
| | GCCGCCGTGAGGAAGACAGATAGCTACCTCTTCGACGCCCAGAGCTTC | |
| | ACATACTGGGGCCAAGGCACACAAGTGACAGTCTCGAGCGGCGGAGGA | |
| | TCCCAAGTGCAGCTGCAAGAGAGCGGAGGCGGCAGCGTGCAGACTGGA | |
| | GGCTCTCTGAGACTGAGCTGTGCTGCCAGCGGCTACACTTATCTGAGG | |
| | GGCTGTATGGGCTGGTTTAGGCAAGCCCCCGGCAAGGAGAGAGAGGGC | |
| | GTGGCCGTCATGGATGTGGTGGGCGATAGGAGAAGCTACATCGACAGC | |
| | GTGAAGGGAAGGTTCACAATCTCTAGGGACAATGCCGCCAACAGCGTC | |
| | TATCTGCAGATGGACAATCTGAAGCCAGAGGACACAGCCATGTACTAC | |
| | TGCACTGCCGGCCCTAACTGTGTGGGCTGGAGAAGCGGACTGGATTAC | |
| | TGGGGCCAAGGCACACAAGTGACAGTCTCGTCTGCTAGCCACCATCAC | |
| | CATCACCAC | |
| 263 | CAAGTGCAGCTGCAAGAGAGCGGAGGAGGAAGCGTGCAAGCCGGAGGC | 361 |
| | TCTCTGAGGCTGAGCTGTGCCGCCTCTAGGTTCACATACAGCAGCTAC | |
| | TGCATGGGCTGGTTCAGACAAGCCCCCGGCAAAGAGAGAGAAGGCGTG | |
| | GCCAGCATCGATAGCGATGGCTCCACTAGCTACACTGACAGCGTGAAG | |
| | GGAAGGTTCACTATCAGCAAGGACAACGCCAAGAACACTCTGTATCTG | |
| | CAGATGAACTCTCTGAAGCCAGAGGACACAGCCATGTACTACTGTGCC | |
| | CTCGATCTGATGAGCACAGTGGTGCCCGGCTTCTGTGGCTTTCTGCTG | |
| | AGCGCTGGCATGGATTACTGGGGCAAGGGCACTCAAGTGACTGTCTCG | |
| | AGCGGCGGAGGATCCCAAGTGCAGCTGCAAGAGAGCGGCGGAGGAAGC | |
| | GTGCAAGCTGGAGGCTCTCTGAGGCTGAGCTGTGCTGCCAGCGGCTAC | |
| | ACTTATAGCAGCGGCTGTATGGGCTGGTTCAGACAAGCCCCCGGCAAG | |
| | GAAAGGGAAGCCGTGGCCGCCATCAATTCCGATGGCAGCACAAGCTAC | |
| | GCCGACAGCGTGAAGGGAAGGTTCACAATCAGCAAGGACAACGCCAAG | |
| | AACACACTCTATCTGCAGATGAACTCTCTGAAGCCAGAGGACACAGCC | |
| | ATGTACTACTGCGCCGCTGAGCCTTACTGTAGCGGCGGCTACCCAAGA | |
| | TGGAGCGTCGCTGAGTTCGGCTACTGGGGCCAAGGCACACAAGTGACT | |
| | GTCTCGTCTGCTAGCCACCATCACCATCACCAC | |
| 264 | CAAGTGCAGCTGCAAGAGAGCGGAGGAGGAAGCGTGCAAGCCGGAGGC | 362 |
| | TCTCTGAGGCTGAGCTGTGCCGCCTCTAGGTTCACATACAGCAGCTAC | |
| | TGCATGGGCTGGTTCAGACAAGCCCCCGGCAAAGAGAGAGAAGGCGTG | |
| | GCCAGCATCGATAGCGATGGCTCCACTAGCTACACTGACAGCGTGAAG | |
| | GGAAGGTTCACTATCAGCAAGGACAACGCCAAGAACACTCTGTATCTG | |
| | CAGATGAACTCTCTGAAGCCAGAGGACACAGCCATGTACTACTGTGCC | |
| | CTCGATCTGATGAGCACAGTGGTGCCCGGCTTCTGTGGCTTTCTGCTG | |
| | AGCGCTGGCATGGATTACTGGGGCAAGGGCACTCAAGTGACTGTCTCG | |
| | AGCGGCGGAGGATCCCAAGTGCAGCTGCAAGAGAGCGGAGGAGGCAGC | |
| | GTCCAAGCCGGAGGCTCTCTGAGGCTGAGCTGTGCTGCCAGCGGCTAC | |
| | ACTTACAGCAGCTACTGCATGGGCTGGTTCAGACAAGCCCCCGGCAAG | |
| | GAGAGAGAGGGCGTGGCTCACATCGACAGCGACGGCTCCACAAGCTAC | |
| | GCCGATAGCGTGAAGGGAAGGTTCACAATCTCCAAGGACAACGCCAAG | |
| | AACACTCTGTACCTCCAGATGAACTCTCTGAAGCCAGAGGACACTGCC | |
| | ATGTACTACTGTGCCGCCGATCCAATTCCCGGCCCCGGCTACTGCGAT | |
| | GGCGGCCCTAACAAGTACTGGGGCCAAGGCACACAAGTGACTGTCTCG | |
| | TCTGCTAGCCACCATCACCATCACCAC | |
| 265 | CAAGTGCAGCTGCAAGAGAGCGGAGGAGGAAGCGTGCAAGCCGGAGGC | 363 |
| | TCTCTGAGGCTGAGCTGTGCCGCCTCTAGGTTCACATACAGCAGCTAC | |
| | TGCATGGGCTGGTTCAGACAAGCCCCCGGCAAAGAGAGAGAAGGCGTG | |
| | GCCAGCATCGATAGCGATGGCTCCACTAGCTACACTGACAGCGTGAAG | |
| | GGAAGGTTCACTATCAGCAAGGACAACGCCAAGAACACTCTGTATCTG | |
| | CAGATGAACTCTCTGAAGCCAGAGGACACAGCCATGTACTACTGTGCC | |
| | CTCGATCTGATGAGCACAGTGGTGCCCGGCTTCTGTGGCTTTCTGCTG | |
| | AGCGCTGGCATGGATTACTGGGGCAAGGGCACTCAAGTGACTGTCTCG | |
| | AGCGGCGGAGGATCCCAAGTGCAGCTGCAAGAGTCCGGAGGAGGAAGC | |
| | GTGCAAGCCGGCGGATCTCTGAGACTGAGCTGTGCCGCCTCTAGGTAC | |
| | ACTTACAACAGCTACTGCATGGGCTGGTTCAGACAAGCCCCCGGCAAG | |
| | GAAAGAGAGGGCGTGGCCACTATCGATAGCGACGGCATGACTAGGTAC | |
| | GCTGATAGCGTCAAGGGAAGGTTCACAATCTCCAAGGACAATGCTAAG | |
| | AACACTCTGTACCTCCAGATGAACTCTCTGAAGCCAGAGGACACAGCC | |
| | ATGTACTACTGCGCTGCCGATGCCGACTGCACTATCGCCGCCATGACT | |
| | ACTAATCCTCTGGGCCAAGGCACACAAGTGACTGTCTCGTCTGCTAGC | |
| | CACCATCACCATCACCAC | |
| 266 | CAAGTGCAGCTGCAAGAGAGCGGAGGAGGAAGCGTGCAAGCCGGAGGC | 364 |
| | TCTCTGAGGCTGAGCTGTGCCGCCTCTAGGTTCACATACAGCAGCTAC | |
| | TGCATGGGCTGGTTCAGACAAGCCCCCGGCAAAGAGAGAGAAGGCGTG | |
| | GCCAGCATCGATAGCGATGGCTCCACTAGCTACACTGACAGCGTGAAG | |
| | GGAAGGTTCACTATCAGCAAGGACAACGCCAAGAACACTCTGTATCTG | |
| | CAGATGAACTCTCTGAAGCCAGAGGACACAGCCATGTACTACTGTGCC | |
| | CTCGATCTGATGAGCACAGTGGTGCCCGGCTTCTGTGGCTTTCTGCTG | |
| | AGCGCTGGCATGGATTACTGGGGCAAGGGCACTCAAGTGACTGTCTCG | |
| | AGCGGCGGAGGATCCCAAGTGCAGCTGCAAGAGTCCGGCGGAGGCAGC | |
| | GTCCAAGCCGGAGGATCTCTGAGGCTGAGCTGTACAGTGAGCAGATAC | |
| | ACTGCCAGCGTGAACTACATGGGCTGGTTCAGACAAGCCCCCGGCAAA | |
| | GAGAGAGAGGGCGTGGCCACAATCTTCACTGGCGCCGGCACAACATAC | |
| | TACGCCAACTCCGTCAAGGGAAGGTTCACAATCTCTAGGGACAACGCC | |
| | AAGAACACTGCCTATCTGCAGATGAACTCCCTCAAGCCAGAGGACACT | |
| | GCCATCTACTACTGCGCCGTGGATTTCAGAGGCGGACTGCTGTATAGG | |
| | CCAGCCTACGAGTACACTTATAGGGGCCAAGGCACACAAGTGACAGTC | |
| | TCGTCTGCTAGCCACCATCACCATCACCAC | |
| 267 | CAAGTGCAGCTGCAAGAGAGCGGAGGAGGAAGCGTGCAAGCCGGAGGC | 365 |
| | TCTCTGAGGCTGAGCTGTGCCGCCTCTAGGTTCACATACAGCAGCTAC | |
| | TGCATGGGCTGGTTCAGACAAGCCCCCGGCAAAGAGAGAGAAGGCGTG | |
| | GCCAGCATCGATAGCGATGGCTCCACTAGCTACACTGACAGCGTGAAG | |
| | GGAAGGTTCACTATCAGCAAGGACAACGCCAAGAACACTCTGTATCTG | |
| | CAGATGAACTCTCTGAAGCCAGAGGACACAGCCATGTACTACTGTGCC | |
| | CTCGATCTGATGAGCACAGTGGTGCCCGGCTTCTGTGGCTTTCTGCTG | |
| | AGCGCTGGCATGGATTACTGGGGCAAGGGCACTCAAGTGACTGTCTCG | |
| | AGCGGCGGAGGATCCCAAGTGCAGCTGCAAGAGAGCGGAGGAGGCAGC | |
| | GTCGAAGCTGGAGGATCTCTGAGGCTGAGCTGTGCTGCCAGCGGCTAC | |
| | ACTCACAGCAGCTACTGTATGGGCTGGTTCAGACAAGCCCCCGGCAAG | |
| | GAGAGGGAAGGCGTGGCTGCCATCGACGTGGATGGCAGCACTACTTAC | |
| | GCCGACAGCGTGAAGGGAAGGTTCACTATCAGCAAGGACAACGCCAAG | |
| | AACACACTCTATCTGCAGATGAACAGCCTCAAGCCAGAGGACACTGGC | |
| | ATGTACTACTGCGCCGCCGAGTTCGCCGATTGCAGCAGCAACTACTTT | |
| | CTGCCTCCCGGCGCCGTCAGATATTGGGGCCAAGGCACTCAAGTGACA | |
| | GTCTCGTCTGCTAGCCACCATCACCATCACCAC | |
| 268 | CAAGTGCAGCTGCAAGAGAGCGGAGGAGGAAGCGTGCAAGCCGGAGGC | 366 |
| | TCTCTGAGGCTGAGCTGTGCCGCCTCTAGGTTCACATACAGCAGCTAC | |
| | TGCATGGGCTGGTTCAGACAAGCCCCCGGCAAAGAGAGAGAAGGCGTG | |
| | GCCAGCATCGATAGCGATGGCTCCACTAGCTACACTGACAGCGTGAAG | |
| | GGAAGGTTCACTATCAGCAAGGACAACGCCAAGAACACTCTGTATCTG | |
| | CAGATGAACTCTCTGAAGCCAGAGGACACAGCCATGTACTACTGTGCC | |
| | CTCGATCTGATGAGCACAGTGGTGCCCGGCTTCTGTGGCTTTCTGCTG | |
| | AGCGCTGGCATGGATTACTGGGGCAAGGGCACTCAAGTGACTGTCTCG | |
| | AGCGGCGGAGGATCCCAAGTGCAGCTGCAAGAGAGCGGAGGAGGAAGC | |
| | GTCCAAGCCGGAGGATCTCTGAGACTGAGCTGCGCCGCTAGTGGCTAC | |
| | TCCTACAGCAGCTACTGCATGGGCTGGTTTAGGCAAGCCCCCGGCAAG | |
| | GAGAGAGAAGGCGTGGCCACTATCGACAGCGACGGCATGACAAGGTAC | |
| | GCCGACAGCGTGAAGGGAAGGTTCACAATCAGCAAGGACAACGCCAAG | |
| | AACACACTGTATCTGCAGATGAACTCTCTGAAGCCAGAGGACACTGCC | |
| | ATGTACTACTGTGCCGCTCCTCTGTACGACTGTGATAGCGGCGCTGTG | |
| | GGCAGAAATCCACCTTATTGGGGCCAAGGCACTCAAGTGACAGTCTCG | |
| | TCTGCTAGCCACCATCACCATCACCAC | |
| 269 | CAAGTGCAGCTGCAAGAGAGCGGAGGAGGAAGCGTGCAAGCCGGAGGC | 367 |
| | TCTCTGAGGCTGAGCTGTGCCGCCTCTAGGTTCACATACAGCAGCTAC | |
| | TGCATGGGCTGGTTCAGACAAGCCCCCGGCAAAGAGAGAGAAGGCGTG | |
| | GCCAGCATCGATAGCGATGGCTCCACTAGCTACACTGACAGCGTGAAG | |
| | GGAAGGTTCACTATCAGCAAGGACAACGCCAAGAACACTCTGTATCTG | |
| | CAGATGAACTCTCTGAAGCCAGAGGACACAGCCATGTACTACTGTGCC | |
| | CTCGATCTGATGAGCACAGTGGTGCCCGGCTTCTGTGGCTTTCTGCTG | |
| | AGCGCTGGCATGGATTACTGGGGCAAGGGCACTCAAGTGACTGTCTCG | |
| | AGCGGCGGAGGATCCCAAGTGCAGCTGCAAGAGAGCGGAGGCGGCAGC | |
| | GTGCAGACTGGAGGCTCTCTGAGACTGAGCTGTGCTGCCAGCGGCTAC | |
| | ACTTATCTGAGGGGCTGTATGGGCTGGTTTAGGCAAGCCCCCGGCAAG | |
| | GAGAGAGAGGGCGTGGCCGTCATGGATGTGGTGGGCGATAGGAGAAGC | |
| | TACATCGACAGCGTGAAGGGAAGGTTCACAATCTCTAGGGACAATGCC | |
| | GCCAACAGCGTCTATCTGCAGATGGACAATCTGAAGCCAGAGGACACA | |
| | GCCATGTACTACTGCACTGCCGGCCCTAACTGTGTGGGCTGGAGAAGC | |
| | GGACTGGATTACTGGGGCCAAGGCACACAAGTGACAGTCTCGTCTGCT | |
| | AGCCACCATCACCATCACCAC | |
| 270 | CAAGTGCAGCTGCAAGAGTCCGGAGGAGGCAGCGTCCAAGCCGGAGGC | 368 |
| | TCTCTGAGGCTGAGCTGTGCTGCCAGCGGCTACACTTACAGCATGTAC | |
| | TGCATGGGCTGGTTCAGACAAGCCCCCGGCAAGGAAAGAGAGGGCGTG | |
| | GCCCAGATCAATAGCGATGGCAGCACAAGCTACGCCGACAGCGTGAAG | |
| | GGAAGGTTCACTATCTCCAAGGACAACGCCAAGAACACTCTGTATCTG | |
| | CAGATGAACTCTCTGAAGCCAGAGGACACTGCCATGTACTACTGCGCT | |
| | GCCGATTCTAGGGTGTACGGCGGCAGCTGGTATGAGAGGCTCTGCGGC | |
| | CCTTACACATACGAGTACAACTACTGGGGCCAAGGCACACAAGTGACT | |
| | GTCTCGAGCGGCGGAGGATCCCAAGTGCAGCTGCAAGAGAGCGGCGGA | |
| | GGAAGCGTGCAAGCTGGAGGCTCTCTGAGGCTGAGCTGTGCTGCCAGC | |
| | GGCTACACTTATAGCAGCGGCTGTATGGGCTGGTTCAGACAAGCCCCC | |
| | GGCAAGGAAAGGGAAGCCGTGGCCGCCATCAATTCCGATGGCAGCACA | |
| | AGCTACGCCGACAGCGTGAAGGGAAGGTTCACAATCAGCAAGGACAAC | |
| | GCCAAGAACACACTCTATCTGCAGATGAACTCTCTGAAGCCAGAGGAC | |
| | ACAGCCATGTACTACTGCGCCGCTGAGCCTTACTGTAGCGGCGGCTAC | |
| | CCAAGATGGAGCGTCGCTGAGTTCGGCTACTGGGGCCAAGGCACACAA | |
| | GTGACTGTCTCGTCTGCTAGCCACCATCACCATCACCAC | |
| 271 | CAAGTGCAGCTGCAAGAGTCCGGAGGAGGCAGCGTCCAAGCCGGAGGC | 369 |
| | TCTCTGAGGCTGAGCTGTGCTGCCAGCGGCTACACTTACAGCATGTAC | |
| | TGCATGGGCTGGTTCAGACAAGCCCCCGGCAAGGAAAGAGAGGGCGTG | |
| | GCCCAGATCAATAGCGATGGCAGCACAAGCTACGCCGACAGCGTGAAG | |
| | GGAAGGTTCACTATCTCCAAGGACAACGCCAAGAACACTCTGTATCTG | |
| | CAGATGAACTCTCTGAAGCCAGAGGACACTGCCATGTACTACTGCGCT | |
| | GCCGATTCTAGGGTGTACGGCGGCAGCTGGTATGAGAGGCTCTGCGGC | |
| | CCTTACACATACGAGTACAACTACTGGGGCCAAGGCACACAAGTGACT | |
| | GTCTCGAGCGGCGGAGGATCCCAAGTGCAGCTGCAAGAGAGCGGAGGA | |
| | GGCAGCGTCCAAGCCGGAGGCTCTCTGAGGCTGAGCTGTGCTGCCAGC | |
| | GGCTACACTTACAGCAGCTACTGCATGGGCTGGTTCAGACAAGCCCCC | |
| | GGCAAGGAGAGAGAGGGCGTGGCTCACATCGACAGCGACGGCTCCACA | |
| | AGCTACGCCGATAGCGTGAAGGGAAGGTTCACAATCTCCAAGGACAAC | |
| | GCCAAGAACACTCTGTACCTCCAGATGAACTCTCTGAAGCCAGAGGAC | |
| | ACTGCCATGTACTACTGTGCCGCCGATCCAATTCCCGGCCCCGGCTAC | |
| | TGCGATGGCGGCCCTAACAAGTACTGGGGCCAAGGCACACAAGTGACT | |
| | GTCTCGTCTGCTAGCCACCATCACCATCACCAC | |
| 272 | CAAGTGCAGCTGCAAGAGTCCGGAGGAGGCAGCGTCCAAGCCGGAGGC | 370 |
| | TCTCTGAGGCTGAGCTGTGCTGCCAGCGGCTACACTTACAGCATGTAC | |
| | TGCATGGGCTGGTTCAGACAAGCCCCCGGCAAGGAAAGAGAGGGCGTG | |
| | GCCCAGATCAATAGCGATGGCAGCACAAGCTACGCCGACAGCGTGAAG | |
| | GGAAGGTTCACTATCTCCAAGGACAACGCCAAGAACACTCTGTATCTG | |
| | CAGATGAACTCTCTGAAGCCAGAGGACACTGCCATGTACTACTGCGCT | |
| | GCCGATTCTAGGGTGTACGGCGGCAGCTGGTATGAGAGGCTCTGCGGC | |
| | CCTTACACATACGAGTACAACTACTGGGGCCAAGGCACACAAGTGACT | |
| | GTCTCGAGCGGCGGAGGATCCCAAGTGCAGCTGCAAGAGTCCGGAGGA | |
| | GGAAGCGTGCAAGCCGGCGGATCTCTGAGACTGAGCTGTGCCGCCTCT | |
| | AGGTACACTTACAACAGCTACTGCATGGGCTGGTTCAGACAAGCCCCC | |
| | GGCAAGGAAAGAGAGGGCGTGGCCACTATCGATAGCGACGGCATGACT | |
| | AGGTACGCTGATAGCGTCAAGGGAAGGTTCACAATCTCCAAGGACAAT | |
| | GCTAAGAACACTCTGTACCTCCAGATGAACTCTCTGAAGCCAGAGGAC | |
| | ACAGCCATGTACTACTGCGCTGCCGATGCCGACTGCACTATCGCCGCC | |
| | ATGACTACTAATCCTCTGGGCCAAGGCACACAAGTGACTGTCTCGTCT | |
| | GCTAGCCACCATCACCATCACCAC | |
| 273 | CAAGTGCAGCTGCAAGAGTCCGGAGGAGGCAGCGTCCAAGCCGGAGGC | 371 |
| | TCTCTGAGGCTGAGCTGTGCTGCCAGCGGCTACACTTACAGCATGTAC | |
| | TGCATGGGCTGGTTCAGACAAGCCCCCGGCAAGGAAAGAGAGGGCGTG | |
| | GCCCAGATCAATAGCGATGGCAGCACAAGCTACGCCGACAGCGTGAAG | |
| | GGAAGGTTCACTATCTCCAAGGACAACGCCAAGAACACTCTGTATCTG | |
| | CAGATGAACTCTCTGAAGCCAGAGGACACTGCCATGTACTACTGCGCT | |
| | GCCGATTCTAGGGTGTACGGCGGCAGCTGGTATGAGAGGCTCTGCGGC | |
| | CCTTACACATACGAGTACAACTACTGGGGCCAAGGCACACAAGTGACT | |
| | GTCTCGAGCGGCGGAGGATCCCAAGTGCAGCTGCAAGAGTCCGGCGGA | |
| | GGCAGCGTCCAAGCCGGAGGATCTCTGAGGCTGAGCTGTACAGTGAGC | |
| | AGATACACTGCCAGCGTGAACTACATGGGCTGGTTCAGACAAGCCCCC | |
| | GGCAAAGAGAGAGAGGGCGTGGCCACAATCTTCACTGGCGCCGGCACA | |
| | ACATACTACGCCAACTCCGTCAAGGGAAGGTTCACAATCTCTAGGGAC | |
| | AACGCCAAGAACACTGCCTATCTGCAGATGAACTCCCTCAAGCCAGAG | |
| | GACACTGCCATCTACTACTGCGCCGTGGATTTCAGAGGCGGACTGCTG | |
| | TATAGGCCAGCCTACGAGTACACTTATAGGGGCCAAGGCACACAAGTG | |
| | ACAGTCTCGTCTGCTAGCCACCATCACCATCACCAC | |
| 274 | CAAGTGCAGCTGCAAGAGTCCGGAGGAGGCAGCGTCCAAGCCGGAGGC | 372 |
| | TCTCTGAGGCTGAGCTGTGCTGCCAGCGGCTACACTTACAGCATGTAC | |
| | TGCATGGGCTGGTTCAGACAAGCCCCCGGCAAGGAAAGAGAGGGCGTG | |
| | GCCCAGATCAATAGCGATGGCAGCACAAGCTACGCCGACAGCGTGAAG | |
| | GGAAGGTTCACTATCTCCAAGGACAACGCCAAGAACACTCTGTATCTG | |
| | CAGATGAACTCTCTGAAGCCAGAGGACACTGCCATGTACTACTGCGCT | |
| | GCCGATTCTAGGGTGTACGGCGGCAGCTGGTATGAGAGGCTCTGCGGC | |
| | CCTTACACATACGAGTACAACTACTGGGGCCAAGGCACACAAGTGACT | |
| | GTCTCGAGCGGCGGAGGATCCCAAGTGCAGCTGCAAGAGAGCGGAGGA | |
| | GGCAGCGTCGAAGCTGGAGGATCTCTGAGGCTGAGCTGTGCTGCCAGC | |
| | GGCTACACTCACAGCAGCTACTGTATGGGCTGGTTCAGACAAGCCCCC | |
| | GGCAAGGAGAGGGAAGGCGTGGCTGCCATCGACGTGGATGGCAGCACT | |
| | ACTTACGCCGACAGCGTGAAGGGAAGGTTCACTATCAGCAAGGACAAC | |
| | GCCAAGAACACACTCTATCTGCAGATGAACAGCCTCAAGCCAGAGGAC | |
| | ACTGGCATGTACTACTGCGCCGCCGAGTTCGCCGATTGCAGCAGCAAC | |
| | TACTTTCTGCCTCCCGGCGCCGTCAGATATTGGGGCCAAGGCACTCAA | |
| | GTGACAGTCTCGTCTGCTAGCCACCATCACCATCACCAC | |
| 275 | CAAGTGCAGCTGCAAGAGTCCGGAGGAGGCAGCGTCCAAGCCGGAGGC | 373 |
| | TCTCTGAGGCTGAGCTGTGCTGCCAGCGGCTACACTTACAGCATGTAC | |
| | TGCATGGGCTGGTTCAGACAAGCCCCCGGCAAGGAAAGAGAGGGCGTG | |
| | GCCCAGATCAATAGCGATGGCAGCACAAGCTACGCCGACAGCGTGAAG | |
| | GGAAGGTTCACTATCTCCAAGGACAACGCCAAGAACACTCTGTATCTG | |
| | CAGATGAACTCTCTGAAGCCAGAGGACACTGCCATGTACTACTGCGCT | |
| | GCCGATTCTAGGGTGTACGGCGGCAGCTGGTATGAGAGGCTCTGCGGC | |
| | CCTTACACATACGAGTACAACTACTGGGGCCAAGGCACACAAGTGACT | |
| | GTCTCGAGCGGCGGAGGATCCCAAGTGCAGCTGCAAGAGAGCGGAGGA | |
| | GGAAGCGTCCAAGCCGGAGGATCTCTGAGACTGAGCTGCGCCGCTAGT | |
| | GGCTACTCCTACAGCAGCTACTGCATGGGCTGGTTTAGGCAAGCCCCC | |
| | GGCAAGGAGAGAGAAGGCGTGGCCACTATCGACAGCGACGGCATGACA | |
| | AGGTACGCCGACAGCGTGAAGGGAAGGTTCACAATCAGCAAGGACAAC | |
| | GCCAAGAACACACTGTATCTGCAGATGAACTCTCTGAAGCCAGAGGAC | |
| | ACTGCCATGTACTACTGTGCCGCTCCTCTGTACGACTGTGATAGCGGC | |
| | GCTGTGGGCAGAAATCCACCTTATTGGGGCCAAGGCACTCAAGTGACA | |
| | GTCTCGTCTGCTAGCCACCATCACCATCACCAC | |
| 276 | CAAGTGCAGCTGCAAGAGTCCGGAGGAGGCAGCGTCCAAGCCGGAGGC | 374 |
| | TCTCTGAGGCTGAGCTGTGCTGCCAGCGGCTACACTTACAGCATGTAC | |
| | TGCATGGGCTGGTTCAGACAAGCCCCCGGCAAGGAAAGAGAGGGCGTG | |
| | GCCCAGATCAATAGCGATGGCAGCACAAGCTACGCCGACAGCGTGAAG | |
| | GGAAGGTTCACTATCTCCAAGGACAACGCCAAGAACACTCTGTATCTG | |
| | CAGATGAACTCTCTGAAGCCAGAGGACACTGCCATGTACTACTGCGCT | |
| | GCCGATTCTAGGGTGTACGGCGGCAGCTGGTATGAGAGGCTCTGCGGC | |
| | CCTTACACATACGAGTACAACTACTGGGGCCAAGGCACACAAGTGACT | |
| | GTCTCGAGCGGCGGAGGATCCCAAGTGCAGCTGCAAGAGAGCGGAGGC | |
| | GGCAGCGTGCAGACTGGAGGCTCTCTGAGACTGAGCTGTGCTGCCAGC | |
| | GGCTACACTTATCTGAGGGGCTGTATGGGCTGGTTTAGGCAAGCCCCC | |
| | GGCAAGGAGAGAGAGGGCGTGGCCGTCATGGATGTGGTGGGCGATAGG | |
| | AGAAGCTACATCGACAGCGTGAAGGGAAGGTTCACAATCTCTAGGGAC | |
| | AATGCCGCCAACAGCGTCTATCTGCAGATGGACAATCTGAAGCCAGAG | |
| | GACACAGCCATGTACTACTGCACTGCCGGCCCTAACTGTGTGGGCTGG | |
| | AGAAGCGGACTGGATTACTGGGGCCAAGGCACACAAGTGACAGTCTCG | |
| | TCTGCTAGCCACCATCACCATCACCAC | |
| 277 | CAAGTGCAGCTGCAAGAGAGCGGCGGAGGAAGCGTGCAAGCCGGAGGA | 375 |
| | TCTCTGAGACTGAGCTGCGCTGTGAGCGGCTACGCCTACTCCACATAC | |
| | TGCATGGGCTGGTTTAGGCAAGCCCCCGGCAAAGAGAGAGAGGGCGTG | |
| | GCTGCTATCGATAGCGGCGGCAGCACAAGCTACGCCGATAGCGTGAAG | |
| | GGAAGGTTCACAATCAGCAAGGACAACGCCAAGAACACACTGTATCTG | |
| | AGGATGAACTCTCTGAAGCCAGAGGACACAGCCATGTACTACTGTGCT | |
| | GCTGTGCCTCCTCCTCCAGATGGCGGCAGCTGTCTGTTTCTGGGACCA | |
| | GAGATCAAGGTCAGCAAGGCCGATTTTAGGTACTGGGGCCAAGGCACA | |
| | CAAGTGACAGTCTCGAGCGGCGGAGGATCCCAAGTGCAGCTGCAAGAG | |
| | AGCGGCGGAGGAAGCGTGCAAGCTGGAGGCTCTCTGAGGCTGAGCTGT | |
| | GCTGCCAGCGGCTACACTTATAGCAGCGGCTGTATGGGCTGGTTCAGA | |
| | CAAGCCCCCGGCAAGGAAAGGGAAGCCGTGGCCGCCATCAATTCCGAT | |
| | GGCAGCACAAGCTACGCCGACAGCGTGAAGGGAAGGTTCACAATCAGC | |
| | AAGGACAACGCCAAGAACACACTCTATCTGCAGATGAACTCTCTGAAG | |
| | CCAGAGGACACAGCCATGTACTACTGCGCCGCTGAGCCTTACTGTAGC | |
| | GGCGGCTACCCAAGATGGAGCGTCGCTGAGTTCGGCTACTGGGGCCAA | |
| | GGCACACAAGTGACTGTCTCGTCTGCTAGCCACCATCACCATCACCAC | |
| 278 | CAAGTGCAGCTGCAAGAGAGCGGCGGAGGAAGCGTGCAAGCCGGAGGA | 376 |
| | TCTCTGAGACTGAGCTGCGCTGTGAGCGGCTACGCCTACTCCACATAC | |
| | TGCATGGGCTGGTTTAGGCAAGCCCCCGGCAAAGAGAGAGAGGGCGTG | |
| | GCTGCTATCGATAGCGGCGGCAGCACAAGCTACGCCGATAGCGTGAAG | |
| | GGAAGGTTCACAATCAGCAAGGACAACGCCAAGAACACACTGTATCTG | |
| | AGGATGAACTCTCTGAAGCCAGAGGACACAGCCATGTACTACTGTGCT | |
| | GCTGTGCCTCCTCCTCCAGATGGCGGCAGCTGTCTGTTTCTGGGACCA | |
| | GAGATCAAGGTCAGCAAGGCCGATTTTAGGTACTGGGGCCAAGGCACA | |
| | CAAGTGACAGTCTCGAGCGGCGGAGGATCCCAAGTGCAGCTGCAAGAG | |
| | AGCGGAGGAGGCAGCGTCCAAGCCGGAGGCTCTCTGAGGCTGAGCTGT | |
| | GCTGCCAGCGGCTACACTTACAGCAGCTACTGCATGGGCTGGTTCAGA | |
| | CAAGCCCCCGGCAAGGAGAGAGAGGGCGTGGCTCACATCGACAGCGAC | |
| | GGCTCCACAAGCTACGCCGATAGCGTGAAGGGAAGGTTCACAATCTCC | |
| | AAGGACAACGCCAAGAACACTCTGTACCTCCAGATGAACTCTCTGAAG | |
| | CCAGAGGACACTGCCATGTACTACTGTGCCGCCGATCCAATTCCCGGC | |
| | CCCGGCTACTGCGATGGCGGCCCTAACAAGTACTGGGGCCAAGGCACA | |
| | CAAGTGACTGTCTCGTCTGCTAGCCACCATCACCATCACCAC | |
| 279 | CAAGTGCAGCTGCAAGAGAGCGGCGGAGGAAGCGTGCAAGCCGGAGGA | 377 |
| | TCTCTGAGACTGAGCTGCGCTGTGAGCGGCTACGCCTACTCCACATAC | |
| | TGCATGGGCTGGTTTAGGCAAGCCCCCGGCAAAGAGAGAGAGGGCGTG | |
| | GCTGCTATCGATAGCGGCGGCAGCACAAGCTACGCCGATAGCGTGAAG | |
| | GGAAGGTTCACAATCAGCAAGGACAACGCCAAGAACACACTGTATCTG | |
| | AGGATGAACTCTCTGAAGCCAGAGGACACAGCCATGTACTACTGTGCT | |
| | GCTGTGCCTCCTCCTCCAGATGGCGGCAGCTGTCTGTTTCTGGGACCA | |
| | GAGATCAAGGTCAGCAAGGCCGATTTTAGGTACTGGGGCCAAGGCACA | |
| | CAAGTGACAGTCTCGAGCGGCGGAGGATCCCAAGTGCAGCTGCAAGAG | |
| | TCCGGAGGAGGAAGCGTGCAAGCCGGCGGATCTCTGAGACTGAGCTGT | |
| | GCCGCCTCTAGGTACACTTACAACAGCTACTGCATGGGCTGGTTCAGA | |
| | CAAGCCCCCGGCAAGGAAAGAGAGGGCGTGGCCACTATCGATAGCGAC | |
| | GGCATGACTAGGTACGCTGATAGCGTCAAGGGAAGGTTCACAATCTCC | |
| | AAGGACAATGCTAAGAACACTCTGTACCTCCAGATGAACTCTCTGAAG | |
| | CCAGAGGACACAGCCATGTACTACTGCGCTGCCGATGCCGACTGCACT | |
| | ATCGCCGCCATGACTACTAATCCTCTGGGCCAAGGCACACAAGTGACT | |
| | GTCTCGTCTGCTAGCCACCATCACCATCACCAC | |
| 280 | CAAGTGCAGCTGCAAGAGAGCGGCGGAGGAAGCGTGCAAGCCGGAGGA | 378 |
| | TCTCTGAGACTGAGCTGCGCTGTGAGCGGCTACGCCTACTCCACATAC | |
| | TGCATGGGCTGGTTTAGGCAAGCCCCCGGCAAAGAGAGAGAGGGCGTG | |
| | GCTGCTATCGATAGCGGCGGCAGCACAAGCTACGCCGATAGCGTGAAG | |
| | GGAAGGTTCACAATCAGCAAGGACAACGCCAAGAACACACTGTATCTG | |
| | AGGATGAACTCTCTGAAGCCAGAGGACACAGCCATGTACTACTGTGCT | |
| | GCTGTGCCTCCTCCTCCAGATGGCGGCAGCTGTCTGTTTCTGGGACCA | |
| | GAGATCAAGGTCAGCAAGGCCGATTTTAGGTACTGGGGCCAAGGCACA | |
| | CAAGTGACAGTCTCGAGCGGCGGAGGATCCCAAGTGCAGCTGCAAGAG | |
| | TCCGGCGGAGGCAGCGTCCAAGCCGGAGGATCTCTGAGGCTGAGCTGT | |
| | ACAGTGAGCAGATACACTGCCAGCGTGAACTACATGGGCTGGTTCAGA | |
| | CAAGCCCCCGGCAAAGAGAGAGAGGGCGTGGCCACAATCTTCACTGGC | |
| | GCCGGCACAACATACTACGCCAACTCCGTCAAGGGAAGGTTCACAATC | |
| | TCTAGGGACAACGCCAAGAACACTGCCTATCTGCAGATGAACTCCCTC | |
| | AAGCCAGAGGACACTGCCATCTACTACTGCGCCGTGGATTTCAGAGGC | |
| | GGACTGCTGTATAGGCCAGCCTACGAGTACACTTATAGGGGCCAAGGC | |
| | ACACAAGTGACAGTCTCGTCTGCTAGCCACCATCACCATCACCAC | |
| 281 | CAAGTGCAGCTGCAAGAGAGCGGCGGAGGAAGCGTGCAAGCCGGAGGA | 379 |
| | TCTCTGAGACTGAGCTGCGCTGTGAGCGGCTACGCCTACTCCACATAC | |
| | TGCATGGGCTGGTTTAGGCAAGCCCCCGGCAAAGAGAGAGAGGGCGTG | |
| | GCTGCTATCGATAGCGGCGGCAGCACAAGCTACGCCGATAGCGTGAAG | |
| | GGAAGGTTCACAATCAGCAAGGACAACGCCAAGAACACACTGTATCTG | |
| | AGGATGAACTCTCTGAAGCCAGAGGACACAGCCATGTACTACTGTGCT | |
| | GCTGTGCCTCCTCCTCCAGATGGCGGCAGCTGTCTGTTTCTGGGACCA | |
| | GAGATCAAGGTCAGCAAGGCCGATTTTAGGTACTGGGGCCAAGGCACA | |
| | CAAGTGACAGTCTCGAGCGGCGGAGGATCCCAAGTGCAGCTGCAAGAG | |
| | AGCGGAGGAGGCAGCGTCGAAGCTGGAGGATCTCTGAGGCTGAGCTGT | |
| | GCTGCCAGCGGCTACACTCACAGCAGCTACTGTATGGGCTGGTTCAGA | |
| | CAAGCCCCCGGCAAGGAGAGGGAAGGCGTGGCTGCCATCGACGTGGAT | |
| | GGCAGCACTACTTACGCCGACAGCGTGAAGGGAAGGTTCACTATCAGC | |
| | AAGGACAACGCCAAGAACACACTCTATCTGCAGATGAACAGCCTCAAG | |
| | CCAGAGGACACTGGCATGTACTACTGCGCCGCCGAGTTCGCCGATTGC | |
| | AGCAGCAACTACTTTCTGCCTCCCGGCGCCGTCAGATATTGGGGCCAA | |
| | GGCACTCAAGTGACAGTCTCGTCTGCTAGCCACCATCACCATCACCAC | |
| 282 | CAAGTGCAGCTGCAAGAGAGCGGCGGAGGAAGCGTGCAAGCCGGAGGA | 380 |
| | TCTCTGAGACTGAGCTGCGCTGTGAGCGGCTACGCCTACTCCACATAC | |
| | TGCATGGGCTGGTTTAGGCAAGCCCCCGGCAAAGAGAGAGAGGGCGTG | |
| | GCTGCTATCGATAGCGGCGGCAGCACAAGCTACGCCGATAGCGTGAAG | |
| | GGAAGGTTCACAATCAGCAAGGACAACGCCAAGAACACACTGTATCTG | |
| | AGGATGAACTCTCTGAAGCCAGAGGACACAGCCATGTACTACTGTGCT | |
| | GCTGTGCCTCCTCCTCCAGATGGCGGCAGCTGTCTGTTTCTGGGACCA | |
| | GAGATCAAGGTCAGCAAGGCCGATTTTAGGTACTGGGGCCAAGGCACA | |
| | CAAGTGACAGTCTCGAGCGGCGGAGGATCCCAAGTGCAGCTGCAAGAG | |
| | AGCGGAGGAGGAAGCGTCCAAGCCGGAGGATCTCTGAGACTGAGCTGC | |
| | GCCGCTAGTGGCTACTCCTACAGCAGCTACTGCATGGGCTGGTTTAGG | |
| | CAAGCCCCCGGCAAGGAGAGAGAAGGCGTGGCCACTATCGACAGCGAC | |
| | GGCATGACAAGGTACGCCGACAGCGTGAAGGGAAGGTTCACAATCAGC | |
| | AAGGACAACGCCAAGAACACACTGTATCTGCAGATGAACTCTCTGAAG | |
| | CCAGAGGACACTGCCATGTACTACTGTGCCGCTCCTCTGTACGACTGT | |
| | GATAGCGGCGCTGTGGGCAGAAATCCACCTTATTGGGGCCAAGGCACT | |
| | CAAGTGACAGTCTCGTCTGCTAGCCACCATCACCATCACCAC | |
| 283 | CAAGTGCAGCTGCAAGAGAGCGGCGGAGGAAGCGTGCAAGCCGGAGGA | 381 |
| | TCTCTGAGACTGAGCTGCGCTGTGAGCGGCTACGCCTACTCCACATAC | |
| | TGCATGGGCTGGTTTAGGCAAGCCCCCGGCAAAGAGAGAGAGGGCGTG | |
| | GCTGCTATCGATAGCGGCGGCAGCACAAGCTACGCCGATAGCGTGAAG | |
| | GGAAGGTTCACAATCAGCAAGGACAACGCCAAGAACACACTGTATCTG | |
| | AGGATGAACTCTCTGAAGCCAGAGGACACAGCCATGTACTACTGTGCT | |
| | GCTGTGCCTCCTCCTCCAGATGGCGGCAGCTGTCTGTTTCTGGGACCA | |
| | GAGATCAAGGTCAGCAAGGCCGATTTTAGGTACTGGGGCCAAGGCACA | |
| | CAAGTGACAGTCTCGAGCGGCGGAGGATCCCAAGTGCAGCTGCAAGAG | |
| | AGCGGAGGCGGCAGCGTGCAGACTGGAGGCTCTCTGAGACTGAGCTGT | |
| | GCTGCCAGCGGCTACACTTATCTGAGGGGCTGTATGGGCTGGTTTAGG | |
| | CAAGCCCCCGGCAAGGAGAGAGAGGGCGTGGCCGTCATGGATGTGGTG | |
| | GGCGATAGGAGAAGCTACATCGACAGCGTGAAGGGAAGGTTCACAATC | |
| | TCTAGGGACAATGCCGCCAACAGCGTCTATCTGCAGATGGACAATCTG | |
| | AAGCCAGAGGACACAGCCATGTACTACTGCACTGCCGGCCCTAACTGT | |
| | GTGGGCTGGAGAAGCGGACTGGATTACTGGGGCCAAGGCACACAAGTG | |
| | ACAGTCTCGTCTGCTAGCCACCATCACCATCACCAC | |
| 284 | CAAGTGCAGCTGCAAGAGAGCGGAGGAGGAAGCGTGCAAGCCGGAGGC | 382 |
| | TCTCTGAGGCTGAGCTGTACAGTGTCCGGCTACACTTACAGCTCCAAT | |
| | TGCATGGGCTGGTTTAGGCAAGCCCCCGGCAAGGAAAGAGAGGGCGTG | |
| | GCCACTATCTACACTGGCGGCGGCAACACATACTACGCCGATAGCGTG | |
| | AAGGGAAGGTTCACTATCAGCCAAGATAACGCCAAGAACACAGTGTAT | |
| | CTGCAGATGAACAATCTGAAGCCAGAGGACACTGCCATGTACTACTGT | |
| | GCTGCTGAGCCACTGTCTAGGGTGTACGGCGGCAGCTGCCCAACTCCT | |
| | ACATTCGACTACTGGGGCCAAGGCACACAAGTGACTGTCTCGAGCGGC | |
| | GGAGGATCCCAAGTGCAGCTGCAAGAGAGCGGCGGAGGAAGCGTGCAA | |
| | GCTGGAGGCTCTCTGAGGCTGAGCTGTGCTGCCAGCGGCTACACTTAT | |
| | AGCAGCGGCTGTATGGGCTGGTTCAGACAAGCCCCCGGCAAGGAAAGG | |
| | GAAGCCGTGGCCGCCATCAATTCCGATGGCAGCACAAGCTACGCCGAC | |
| | AGCGTGAAGGGAAGGTTCACAATCAGCAAGGACAACGCCAAGAACACA | |
| | CTCTATCTGCAGATGAACTCTCTGAAGCCAGAGGACACAGCCATGTAC | |
| | TACTGCGCCGCTGAGCCTTACTGTAGCGGCGGCTACCCAAGATGGAGC | |
| | GTCGCTGAGTTCGGCTACTGGGGCCAAGGCACACAAGTGACTGTCTCG | |
| | TCTGCTAGCCACCATCACCATCACCAC | |
| 285 | CAAGTGCAGCTGCAAGAGAGCGGAGGAGGAAGCGTGCAAGCCGGAGGC | 383 |
| | TCTCTGAGGCTGAGCTGTACAGTGTCCGGCTACACTTACAGCTCCAAT | |
| | TGCATGGGCTGGTTTAGGCAAGCCCCCGGCAAGGAAAGAGAGGGCGTG | |
| | GCCACTATCTACACTGGCGGCGGCAACACATACTACGCCGATAGCGTG | |
| | AAGGGAAGGTTCACTATCAGCCAAGATAACGCCAAGAACACAGTGTAT | |
| | CTGCAGATGAACAATCTGAAGCCAGAGGACACTGCCATGTACTACTGT | |
| | GCTGCTGAGCCACTGTCTAGGGTGTACGGCGGCAGCTGCCCAACTCCT | |
| | ACATTCGACTACTGGGGCCAAGGCACACAAGTGACTGTCTCGAGCGGC | |
| | GGAGGATCCCAAGTGCAGCTGCAAGAGAGCGGAGGAGGCAGCGTCCAA | |
| | GCCGGAGGCTCTCTGAGGCTGAGCTGTGCTGCCAGCGGCTACACTTAC | |
| | AGCAGCTACTGCATGGGCTGGTTCAGACAAGCCCCCGGCAAGGAGAGA | |
| | GAGGGCGTGGCTCACATCGACAGCGACGGCTCCACAAGCTACGCCGAT | |
| | AGCGTGAAGGGAAGGTTCACAATCTCCAAGGACAACGCCAAGAACACT | |
| | CTGTACCTCCAGATGAACTCTCTGAAGCCAGAGGACACTGCCATGTAC | |
| | TACTGTGCCGCCGATCCAATTCCCGGCCCCGGCTACTGCGATGGCGGC | |
| | CCTAACAAGTACTGGGGCCAAGGCACACAAGTGACTGTCTCGTCTGCT | |
| | AGCCACCATCACCATCACCAC | |
| 286 | CAAGTGCAGCTGCAAGAGAGCGGAGGAGGAAGCGTGCAAGCCGGAGGC | 384 |
| | TCTCTGAGGCTGAGCTGTACAGTGTCCGGCTACACTTACAGCTCCAAT | |
| | TGCATGGGCTGGTTTAGGCAAGCCCCCGGCAAGGAAAGAGAGGGCGTG | |
| | GCCACTATCTACACTGGCGGCGGCAACACATACTACGCCGATAGCGTG | |
| | AAGGGAAGGTTCACTATCAGCCAAGATAACGCCAAGAACACAGTGTAT | |
| | CTGCAGATGAACAATCTGAAGCCAGAGGACACTGCCATGTACTACTGT | |
| | GCTGCTGAGCCACTGTCTAGGGTGTACGGCGGCAGCTGCCCAACTCCT | |
| | ACATTCGACTACTGGGGCCAAGGCACACAAGTGACTGTCTCGAGCGGC | |
| | GGAGGATCCCAAGTGCAGCTGCAAGAGTCCGGAGGAGGAAGCGTGCAA | |
| | GCCGGCGGATCTCTGAGACTGAGCTGTGCCGCCTCTAGGTACACTTAC | |
| | AACAGCTACTGCATGGGCTGGTTCAGACAAGCCCCCGGCAAGGAAAGA | |
| | GAGGGCGTGGCCACTATCGATAGCGACGGCATGACTAGGTACGCTGAT | |
| | AGCGTCAAGGGAAGGTTCACAATCTCCAAGGACAATGCTAAGAACACT | |
| | CTGTACCTCCAGATGAACTCTCTGAAGCCAGAGGACACAGCCATGTAC | |
| | TACTGCGCTGCCGATGCCGACTGCACTATCGCCGCCATGACTACTAAT | |
| | CCTCTGGGCCAAGGCACACAAGTGACTGTCTCGTCTGCTAGCCACCAT | |
| | CACCATCACCAC | |
| 287 | CAAGTGCAGCTGCAAGAGAGCGGAGGAGGAAGCGTGCAAGCCGGAGGC | 385 |
| | TCTCTGAGGCTGAGCTGTACAGTGTCCGGCTACACTTACAGCTCCAAT | |
| | TGCATGGGCTGGTTTAGGCAAGCCCCCGGCAAGGAAAGAGAGGGCGTG | |
| | GCCACTATCTACACTGGCGGCGGCAACACATACTACGCCGATAGCGTG | |
| | AAGGGAAGGTTCACTATCAGCCAAGATAACGCCAAGAACACAGTGTAT | |
| | CTGCAGATGAACAATCTGAAGCCAGAGGACACTGCCATGTACTACTGT | |
| | GCTGCTGAGCCACTGTCTAGGGTGTACGGCGGCAGCTGCCCAACTCCT | |
| | ACATTCGACTACTGGGGCCAAGGCACACAAGTGACTGTCTCGAGCGGC | |
| | GGAGGATCCCAAGTGCAGCTGCAAGAGTCCGGCGGAGGCAGCGTCCAA | |
| | GCCGGAGGATCTCTGAGGCTGAGCTGTACAGTGAGCAGATACACTGCC | |
| | AGCGTGAACTACATGGGCTGGTTCAGACAAGCCCCCGGCAAAGAGAGA | |
| | GAGGGCGTGGCCACAATCTTCACTGGCGCCGGCACAACATACTACGCC | |
| | AACTCCGTCAAGGGAAGGTTCACAATCTCTAGGGACAACGCCAAGAAC | |
| | ACTGCCTATCTGCAGATGAACTCCCTCAAGCCAGAGGACACTGCCATC | |
| | TACTACTGCGCCGTGGATTTCAGAGGCGGACTGCTGTATAGGCCAGCC | |
| | TACGAGTACACTTATAGGGGCCAAGGCACACAAGTGACAGTCTCGTCT | |
| | GCTAGCCACCATCACCATCACCAC | |
| 288 | CAAGTGCAGCTGCAAGAGAGCGGAGGAGGAAGCGTGCAAGCCGGAGGC | 386 |
| | TCTCTGAGGCTGAGCTGTACAGTGTCCGGCTACACTTACAGCTCCAAT | |
| | TGCATGGGCTGGTTTAGGCAAGCCCCCGGCAAGGAAAGAGAGGGCGTG | |
| | GCCACTATCTACACTGGCGGCGGCAACACATACTACGCCGATAGCGTG | |
| | AAGGGAAGGTTCACTATCAGCCAAGATAACGCCAAGAACACAGTGTAT | |
| | CTGCAGATGAACAATCTGAAGCCAGAGGACACTGCCATGTACTACTGT | |
| | GCTGCTGAGCCACTGTCTAGGGTGTACGGCGGCAGCTGCCCAACTCCT | |
| | ACATTCGACTACTGGGGCCAAGGCACACAAGTGACTGTCTCGAGCGGC | |
| | GGAGGATCCCAAGTGCAGCTGCAAGAGAGCGGAGGAGGCAGCGTCGAA | |
| | GCTGGAGGATCTCTGAGGCTGAGCTGTGCTGCCAGCGGCTACACTCAC | |
| | AGCAGCTACTGTATGGGCTGGTTCAGACAAGCCCCCGGCAAGGAGAGG | |
| | GAAGGCGTGGCTGCCATCGACGTGGATGGCAGCACTACTTACGCCGAC | |
| | AGCGTGAAGGGAAGGTTCACTATCAGCAAGGACAACGCCAAGAACACA | |
| | CTCTATCTGCAGATGAACAGCCTCAAGCCAGAGGACACTGGCATGTAC | |
| | TACTGCGCCGCCGAGTTCGCCGATTGCAGCAGCAACTACTTTCTGCCT | |
| | CCCGGCGCCGTCAGATATTGGGGCCAAGGCACTCAAGTGACAGTCTCG | |
| | TCTGCTAGCCACCATCACCATCACCAC | |
| 289 | CAAGTGCAGCTGCAAGAGAGCGGAGGAGGAAGCGTGCAAGCCGGAGGC | 387 |
| | TCTCTGAGGCTGAGCTGTACAGTGTCCGGCTACACTTACAGCTCCAAT | |
| | TGCATGGGCTGGTTTAGGCAAGCCCCCGGCAAGGAAAGAGAGGGCGTG | |
| | GCCACTATCTACACTGGCGGCGGCAACACATACTACGCCGATAGCGTG | |
| | AAGGGAAGGTTCACTATCAGCCAAGATAACGCCAAGAACACAGTGTAT | |
| | CTGCAGATGAACAATCTGAAGCCAGAGGACACTGCCATGTACTACTGT | |
| | GCTGCTGAGCCACTGTCTAGGGTGTACGGCGGCAGCTGCCCAACTCCT | |
| | ACATTCGACTACTGGGGCCAAGGCACACAAGTGACTGTCTCGAGCGGC | |
| | GGAGGATCCCAAGTGCAGCTGCAAGAGAGCGGAGGAGGAAGCGTCCAA | |
| | GCCGGAGGATCTCTGAGACTGAGCTGCGCCGCTAGTGGCTACTCCTAC | |
| | AGCAGCTACTGCATGGGCTGGTTTAGGCAAGCCCCCGGCAAGGAGAGA | |
| | GAAGGCGTGGCCACTATCGACAGCGACGGCATGACAAGGTACGCCGAC | |
| | AGCGTGAAGGGAAGGTTCACAATCAGCAAGGACAACGCCAAGAACACA | |
| | CTGTATCTGCAGATGAACTCTCTGAAGCCAGAGGACACTGCCATGTAC | |
| | TACTGTGCCGCTCCTCTGTACGACTGTGATAGCGGCGCTGTGGGCAGA | |
| | AATCCACCTTATTGGGGCCAAGGCACTCAAGTGACAGTCTCGTCTGCT | |
| | AGCCACCATCACCATCACCAC | |
| 290 | CAAGTGCAGCTGCAAGAGAGCGGAGGAGGAAGCGTGCAAGCCGGAGGC | 388 |
| | TCTCTGAGGCTGAGCTGTACAGTGTCCGGCTACACTTACAGCTCCAAT | |
| | TGCATGGGCTGGTTTAGGCAAGCCCCCGGCAAGGAAAGAGAGGGCGTG | |
| | GCCACTATCTACACTGGCGGCGGCAACACATACTACGCCGATAGCGTG | |
| | AAGGGAAGGTTCACTATCAGCCAAGATAACGCCAAGAACACAGTGTAT | |
| | CTGCAGATGAACAATCTGAAGCCAGAGGACACTGCCATGTACTACTGT | |
| | GCTGCTGAGCCACTGTCTAGGGTGTACGGCGGCAGCTGCCCAACTCCT | |
| | ACATTCGACTACTGGGGCCAAGGCACACAAGTGACTGTCTCGAGCGGC | |
| | GGAGGATCCCAAGTGCAGCTGCAAGAGAGCGGAGGCGGCAGCGTGCAG | |
| | ACTGGAGGCTCTCTGAGACTGAGCTGTGCTGCCAGCGGCTACACTTAT | |
| | CTGAGGGGCTGTATGGGCTGGTTTAGGCAAGCCCCCGGCAAGGAGAGA | |
| | GAGGGCGTGGCCGTCATGGATGTGGTGGGCGATAGGAGAAGCTACATC | |
| | GACAGCGTGAAGGGAAGGTTCACAATCTCTAGGGACAATGCCGCCAAC | |
| | AGCGTCTATCTGCAGATGGACAATCTGAAGCCAGAGGACACAGCCATG | |
| | TACTACTGCACTGCCGGCCCTAACTGTGTGGGCTGGAGAAGCGGACTG | |
| | GATTACTGGGGCCAAGGCACACAAGTGACAGTCTCGTCTGCTAGCCAC | |
| | CATCACCATCACCAC | |
| 291 | CAAGTGCAGCTGCAAGAGAGCGGAGGAGGAAGCGTCCAAGCCGGAGGC | 389 |
| | TCTCTGAGGCTGAGCTGTGGAGCCAGCGGCTACACTTACAGCAGCTAC | |
| | TGTATGGGCTGGTTTAGGCAAGTGCCCGGCAAGGAGAGAGAGGGCGTG | |
| | GCCGTGATCGATTCCGATGGCAGCACAAGCTACGCTGACAGCGTGAAG | |
| | GGAAGGTTCACAATCAGCAAGGACAACGGCAAGAACACACTCTATCTG | |
| | CAGATGAACAGCCTCAAGCCAGAGGACACAGCCATGTACTACTGCGCC | |
| | GCTGATCTGGGCCACTATAGGCCTCCTTGTGGCGTGCTGTATCTGGGC | |
| | ATGGATTACTGGGGCAAGGGCACACAAGTGACAGTCTCGAGCGGCGGA | |
| | GGATCCCAAGTGCAGCTGCAAGAGAGCGGCGGAGGAAGCGTGCAAGCT | |
| | GGAGGCTCTCTGAGGCTGAGCTGTGCTGCCAGCGGCTACACTTATAGC | |
| | AGCGGCTGTATGGGCTGGTTCAGACAAGCCCCCGGCAAGGAAAGGGAA | |
| | GCCGTGGCCGCCATCAATTCCGATGGCAGCACAAGCTACGCCGACAGC | |
| | GTGAAGGGAAGGTTCACAATCAGCAAGGACAACGCCAAGAACACACTC | |
| | TATCTGCAGATGAACTCTCTGAAGCCAGAGGACACAGCCATGTACTAC | |
| | TGCGCCGCTGAGCCTTACTGTAGCGGCGGCTACCCAAGATGGAGCGTC | |
| | GCTGAGTTCGGCTACTGGGGCCAAGGCACACAAGTGACTGTCTCGTCT | |
| | GCTAGCCACCATCACCATCACCAC | |
| 292 | CAAGTGCAGCTGCAAGAGAGCGGAGGAGGAAGCGTCCAAGCCGGAGGC | 390 |
| | TCTCTGAGGCTGAGCTGTGGAGCCAGCGGCTACACTTACAGCAGCTAC | |
| | TGTATGGGCTGGTTTAGGCAAGTGCCCGGCAAGGAGAGAGAGGGCGTG | |
| | GCCGTGATCGATTCCGATGGCAGCACAAGCTACGCTGACAGCGTGAAG | |
| | GGAAGGTTCACAATCAGCAAGGACAACGGCAAGAACACACTCTATCTG | |
| | CAGATGAACAGCCTCAAGCCAGAGGACACAGCCATGTACTACTGCGCC | |
| | GCTGATCTGGGCCACTATAGGCCTCCTTGTGGCGTGCTGTATCTGGGC | |
| | ATGGATTACTGGGGCAAGGGCACACAAGTGACAGTCTCGAGCGGCGGA | |
| | GGATCCCAAGTGCAGCTGCAAGAGAGCGGAGGAGGCAGCGTCCAAGCC | |
| | GGAGGCTCTCTGAGGCTGAGCTGTGCTGCCAGCGGCTACACTTACAGC | |
| | AGCTACTGCATGGGCTGGTTCAGACAAGCCCCCGGCAAGGAGAGAGAG | |
| | GGCGTGGCTCACATCGACAGCGACGGCTCCACAAGCTACGCCGATAGC | |
| | GTGAAGGGAAGGTTCACAATCTCCAAGGACAACGCCAAGAACACTCTG | |
| | TACCTCCAGATGAACTCTCTGAAGCCAGAGGACACTGCCATGTACTAC | |
| | TGTGCCGCCGATCCAATTCCCGGCCCCGGCTACTGCGATGGCGGCCCT | |
| | AACAAGTACTGGGGCCAAGGCACACAAGTGACTGTCTCGTCTGCTAGC | |
| | CACCATCACCATCACCAC | |
| 293 | CAAGTGCAGCTGCAAGAGAGCGGAGGAGGAAGCGTCCAAGCCGGAGGC | 391 |
| | TCTCTGAGGCTGAGCTGTGGAGCCAGCGGCTACACTTACAGCAGCTAC | |
| | TGTATGGGCTGGTTTAGGCAAGTGCCCGGCAAGGAGAGAGAGGGCGTG | |
| | GCCGTGATCGATTCCGATGGCAGCACAAGCTACGCTGACAGCGTGAAG | |
| | GGAAGGTTCACAATCAGCAAGGACAACGGCAAGAACACACTCTATCTG | |
| | CAGATGAACAGCCTCAAGCCAGAGGACACAGCCATGTACTACTGCGCC | |
| | GCTGATCTGGGCCACTATAGGCCTCCTTGTGGCGTGCTGTATCTGGGC | |
| | ATGGATTACTGGGGCAAGGGCACACAAGTGACAGTCTCGAGCGGCGGA | |
| | GGATCCCAAGTGCAGCTGCAAGAGTCCGGAGGAGGAAGCGTGCAAGCC | |
| | GGCGGATCTCTGAGACTGAGCTGTGCCGCCTCTAGGTACACTTACAAC | |
| | AGCTACTGCATGGGCTGGTTCAGACAAGCCCCCGGCAAGGAAAGAGAG | |
| | GGCGTGGCCACTATCGATAGCGACGGCATGACTAGGTACGCTGATAGC | |
| | GTCAAGGGAAGGTTCACAATCTCCAAGGACAATGCTAAGAACACTCTG | |
| | TACCTCCAGATGAACTCTCTGAAGCCAGAGGACACAGCCATGTACTAC | |
| | TGCGCTGCCGATGCCGACTGCACTATCGCCGCCATGACTACTAATCCT | |
| | CTGGGCCAAGGCACACAAGTGACTGTCTCGTCTGCTAGCCACCATCAC | |
| | CATCACCAC | |
| 294 | CAAGTGCAGCTGCAAGAGAGCGGAGGAGGAAGCGTCCAAGCCGGAGGC | 392 |
| | TCTCTGAGGCTGAGCTGTGGAGCCAGCGGCTACACTTACAGCAGCTAC | |
| | TGTATGGGCTGGTTTAGGCAAGTGCCCGGCAAGGAGAGAGAGGGCGTG | |
| | GCCGTGATCGATTCCGATGGCAGCACAAGCTACGCTGACAGCGTGAAG | |
| | GGAAGGTTCACAATCAGCAAGGACAACGGCAAGAACACACTCTATCTG | |
| | CAGATGAACAGCCTCAAGCCAGAGGACACAGCCATGTACTACTGCGCC | |
| | GCTGATCTGGGCCACTATAGGCCTCCTTGTGGCGTGCTGTATCTGGGC | |
| | ATGGATTACTGGGGCAAGGGCACACAAGTGACAGTCTCGAGCGGCGGA | |
| | GGATCCCAAGTGCAGCTGCAAGAGTCCGGCGGAGGCAGCGTCCAAGCC | |
| | GGAGGATCTCTGAGGCTGAGCTGTACAGTGAGCAGATACACTGCCAGC | |
| | GTGAACTACATGGGCTGGTTCAGACAAGCCCCCGGCAAAGAGAGAGAG | |
| | GGCGTGGCCACAATCTTCACTGGCGCCGGCACAACATACTACGCCAAC | |
| | TCCGTCAAGGGAAGGTTCACAATCTCTAGGGACAACGCCAAGAACACT | |
| | GCCTATCTGCAGATGAACTCCCTCAAGCCAGAGGACACTGCCATCTAC | |
| | TACTGCGCCGTGGATTTCAGAGGCGGACTGCTGTATAGGCCAGCCTAC | |
| | GAGTACACTTATAGGGGCCAAGGCACACAAGTGACAGTCTCGTCTGCT | |
| | AGCCACCATCACCATCACCAC | |
| 295 | CAAGTGCAGCTGCAAGAGAGCGGAGGAGGAAGCGTCCAAGCCGGAGGC | 393 |
| | TCTCTGAGGCTGAGCTGTGGAGCCAGCGGCTACACTTACAGCAGCTAC | |
| | TGTATGGGCTGGTTTAGGCAAGTGCCCGGCAAGGAGAGAGAGGGCGTG | |
| | GCCGTGATCGATTCCGATGGCAGCACAAGCTACGCTGACAGCGTGAAG | |
| | GGAAGGTTCACAATCAGCAAGGACAACGGCAAGAACACACTCTATCTG | |
| | CAGATGAACAGCCTCAAGCCAGAGGACACAGCCATGTACTACTGCGCC | |
| | GCTGATCTGGGCCACTATAGGCCTCCTTGTGGCGTGCTGTATCTGGGC | |
| | ATGGATTACTGGGGCAAGGGCACACAAGTGACAGTCTCGAGCGGCGGA | |
| | GGATCCCAAGTGCAGCTGCAAGAGAGCGGAGGAGGCAGCGTCGAAGCT | |
| | GGAGGATCTCTGAGGCTGAGCTGTGCTGCCAGCGGCTACACTCACAGC | |
| | AGCTACTGTATGGGCTGGTTCAGACAAGCCCCCGGCAAGGAGAGGGAA | |
| | GGCGTGGCTGCCATCGACGTGGATGGCAGCACTACTTACGCCGACAGC | |
| | GTGAAGGGAAGGTTCACTATCAGCAAGGACAACGCCAAGAACACACTC | |
| | TATCTGCAGATGAACAGCCTCAAGCCAGAGGACACTGGCATGTACTAC | |
| | TGCGCCGCCGAGTTCGCCGATTGCAGCAGCAACTACTTTCTGCCTCCC | |
| | GGCGCCGTCAGATATTGGGGCCAAGGCACTCAAGTGACAGTCTCGTCT | |
| | GCTAGCCACCATCACCATCACCAC | |
| 296 | CAAGTGCAGCTGCAAGAGAGCGGAGGAGGAAGCGTCCAAGCCGGAGGC | 394 |
| | TCTCTGAGGCTGAGCTGTGGAGCCAGCGGCTACACTTACAGCAGCTAC | |
| | TGTATGGGCTGGTTTAGGCAAGTGCCCGGCAAGGAGAGAGAGGGCGTG | |
| | GCCGTGATCGATTCCGATGGCAGCACAAGCTACGCTGACAGCGTGAAG | |
| | GGAAGGTTCACAATCAGCAAGGACAACGGCAAGAACACACTCTATCTG | |
| | CAGATGAACAGCCTCAAGCCAGAGGACACAGCCATGTACTACTGCGCC | |
| | GCTGATCTGGGCCACTATAGGCCTCCTTGTGGCGTGCTGTATCTGGGC | |
| | ATGGATTACTGGGGCAAGGGCACACAAGTGACAGTCTCGAGCGGCGGA | |
| | GGATCCCAAGTGCAGCTGCAAGAGAGCGGAGGAGGAAGCGTCCAAGCC | |
| | GGAGGATCTCTGAGACTGAGCTGCGCCGCTAGTGGCTACTCCTACAGC | |
| | AGCTACTGCATGGGCTGGTTTAGGCAAGCCCCCGGCAAGGAGAGAGAA | |
| | GGCGTGGCCACTATCGACAGCGACGGCATGACAAGGTACGCCGACAGC | |
| | GTGAAGGGAAGGTTCACAATCAGCAAGGACAACGCCAAGAACACACTG | |
| | TATCTGCAGATGAACTCTCTGAAGCCAGAGGACACTGCCATGTACTAC | |
| | TGTGCCGCTCCTCTGTACGACTGTGATAGCGGCGCTGTGGGCAGAAAT | |
| | CCACCTTATTGGGGCCAAGGCACTCAAGTGACAGTCTCGTCTGCTAGC | |
| | CACCATCACCATCACCAC | |
| 297 | CAAGTGCAGCTGCAAGAGAGCGGAGGAGGAAGCGTCCAAGCCGGAGGC | 395 |
| | TCTCTGAGGCTGAGCTGTGGAGCCAGCGGCTACACTTACAGCAGCTAC | |
| | TGTATGGGCTGGTTTAGGCAAGTGCCCGGCAAGGAGAGAGAGGGCGTG | |
| | GCCGTGATCGATTCCGATGGCAGCACAAGCTACGCTGACAGCGTGAAG | |
| | GGAAGGTTCACAATCAGCAAGGACAACGGCAAGAACACACTCTATCTG | |
| | CAGATGAACAGCCTCAAGCCAGAGGACACAGCCATGTACTACTGCGCC | |
| | GCTGATCTGGGCCACTATAGGCCTCCTTGTGGCGTGCTGTATCTGGGC | |
| | ATGGATTACTGGGGCAAGGGCACACAAGTGACAGTCTCGAGCGGCGGA | |
| | GGATCCCAAGTGCAGCTGCAAGAGAGCGGAGGCGGCAGCGTGCAGACT | |
| | GGAGGCTCTCTGAGACTGAGCTGTGCTGCCAGCGGCTACACTTATCTG | |
| | AGGGGCTGTATGGGCTGGTTTAGGCAAGCCCCCGGCAAGGAGAGAGAG | |
| | GGCGTGGCCGTCATGGATGTGGTGGGCGATAGGAGAAGCTACATCGAC | |
| | AGCGTGAAGGGAAGGTTCACAATCTCTAGGGACAATGCCGCCAACAGC | |
| | GTCTATCTGCAGATGGACAATCTGAAGCCAGAGGACACAGCCATGTAC | |
| | TACTGCACTGCCGGCCCTAACTGTGTGGGCTGGAGAAGCGGACTGGAT | |
| | TACTGGGGCCAAGGCACACAAGTGACAGTCTCGTCTGCTAGCCACCAT | |
| | CACCATCACCAC | |
| 298 | CAAGTGCAGCTGCAAGAGAGCGGAGGAGGAAGCGTCCAAGCCGGAGGC | 396 |
| | TCTCTGAGACTGAGCTGTGCCGCCAGCGGCTACTCCAACTGCAGCTAC | |
| | GACATGACTTGGTATAGGCAAGCCCCCGGCAAGGAGAGGGAGTTCGTG | |
| | TCCGCCATCCACAGCGACGGCAGCACTAGATACGCCGACAGCGTGAAG | |
| | GGAAGGTTCTTCATCAGCCAAGATAACGCCAAGAACACAGTGTATCTG | |
| | CAGATGAACTCCCTCAAGCCAGAGGACACTGCCATGTACTACTGCAAG | |
| | ACAGACCCACTGCACTGCAGAGCCCATGGCGGCAGCTGGTATAGCGTG | |
| | AGGGCCAACTACTGGGGCCAAGGCACACAAGTGACAGTCTCGAGCGGC | |
| | GGAGGATCCCAAGTGCAGCTGCAAGAGAGCGGCGGAGGAAGCGTGCAA | |
| | GCTGGAGGCTCTCTGAGGCTGAGCTGTGCTGCCAGCGGCTACACTTAT | |
| | AGCAGCGGCTGTATGGGCTGGTTCAGACAAGCCCCCGGCAAGGAAAGG | |
| | GAAGCCGTGGCCGCCATCAATTCCGATGGCAGCACAAGCTACGCCGAC | |
| | AGCGTGAAGGGAAGGTTCACAATCAGCAAGGACAACGCCAAGAACACA | |
| | CTCTATCTGCAGATGAACTCTCTGAAGCCAGAGGACACAGCCATGTAC | |
| | TACTGCGCCGCTGAGCCTTACTGTAGCGGCGGCTACCCAAGATGGAGC | |
| | GTCGCTGAGTTCGGCTACTGGGGCCAAGGCACACAAGTGACTGTCTCG | |
| | TCTGCTAGCCACCATCACCATCACCAC | |
| 299 | CAAGTGCAGCTGCAAGAGAGCGGAGGAGGAAGCGTCCAAGCCGGAGGC | 397 |
| | TCTCTGAGACTGAGCTGTGCCGCCAGCGGCTACTCCAACTGCAGCTAC | |
| | GACATGACTTGGTATAGGCAAGCCCCCGGCAAGGAGAGGGAGTTCGTG | |
| | TCCGCCATCCACAGCGACGGCAGCACTAGATACGCCGACAGCGTGAAG | |
| | GGAAGGTTCTTCATCAGCCAAGATAACGCCAAGAACACAGTGTATCTG | |
| | CAGATGAACTCCCTCAAGCCAGAGGACACTGCCATGTACTACTGCAAG | |
| | ACAGACCCACTGCACTGCAGAGCCCATGGCGGCAGCTGGTATAGCGTG | |
| | AGGGCCAACTACTGGGGCCAAGGCACACAAGTGACAGTCTCGAGCGGC | |
| | GGAGGATCCCAAGTGCAGCTGCAAGAGAGCGGAGGAGGCAGCGTCCAA | |
| | GCCGGAGGCTCTCTGAGGCTGAGCTGTGCTGCCAGCGGCTACACTTAC | |
| | AGCAGCTACTGCATGGGCTGGTTCAGACAAGCCCCCGGCAAGGAGAGA | |
| | GAGGGCGTGGCTCACATCGACAGCGACGGCTCCACAAGCTACGCCGAT | |
| | AGCGTGAAGGGAAGGTTCACAATCTCCAAGGACAACGCCAAGAACACT | |
| | CTGTACCTCCAGATGAACTCTCTGAAGCCAGAGGACACTGCCATGTAC | |
| | TACTGTGCCGCCGATCCAATTCCCGGCCCCGGCTACTGCGATGGCGGC | |
| | CCTAACAAGTACTGGGGCCAAGGCACACAAGTGACTGTCTCGTCTGCT | |
| | AGCCACCATCACCATCACCAC | |
| 300 | CAAGTGCAGCTGCAAGAGAGCGGAGGAGGAAGCGTCCAAGCCGGAGGC | 398 |
| | TCTCTGAGACTGAGCTGTGCCGCCAGCGGCTACTCCAACTGCAGCTAC | |
| | GACATGACTTGGTATAGGCAAGCCCCCGGCAAGGAGAGGGAGTTCGTG | |
| | TCCGCCATCCACAGCGACGGCAGCACTAGATACGCCGACAGCGTGAAG | |
| | GGAAGGTTCTTCATCAGCCAAGATAACGCCAAGAACACAGTGTATCTG | |
| | CAGATGAACTCCCTCAAGCCAGAGGACACTGCCATGTACTACTGCAAG | |
| | ACAGACCCACTGCACTGCAGAGCCCATGGCGGCAGCTGGTATAGCGTG | |
| | AGGGCCAACTACTGGGGCCAAGGCACACAAGTGACAGTCTCGAGCGGC | |
| | GGAGGATCCCAAGTGCAGCTGCAAGAGTCCGGAGGAGGAAGCGTGCAA | |
| | GCCGGCGGATCTCTGAGACTGAGCTGTGCCGCCTCTAGGTACACTTAC | |
| | AACAGCTACTGCATGGGCTGGTTCAGACAAGCCCCCGGCAAGGAAAGA | |
| | GAGGGCGTGGCCACTATCGATAGCGACGGCATGACTAGGTACGCTGAT | |
| | AGCGTCAAGGGAAGGTTCACAATCTCCAAGGACAATGCTAAGAACACT | |
| | CTGTACCTCCAGATGAACTCTCTGAAGCCAGAGGACACAGCCATGTAC | |
| | TACTGCGCTGCCGATGCCGACTGCACTATCGCCGCCATGACTACTAAT | |
| | CCTCTGGGCCAAGGCACACAAGTGACTGTCTCGTCTGCTAGCCACCAT | |
| | CACCATCACCAC | |
| 301 | CAAGTGCAGCTGCAAGAGAGCGGAGGAGGAAGCGTCCAAGCCGGAGGC | 399 |
| | TCTCTGAGACTGAGCTGTGCCGCCAGCGGCTACTCCAACTGCAGCTAC | |
| | GACATGACTTGGTATAGGCAAGCCCCCGGCAAGGAGAGGGAGTTCGTG | |
| | TCCGCCATCCACAGCGACGGCAGCACTAGATACGCCGACAGCGTGAAG | |
| | GGAAGGTTCTTCATCAGCCAAGATAACGCCAAGAACACAGTGTATCTG | |
| | CAGATGAACTCCCTCAAGCCAGAGGACACTGCCATGTACTACTGCAAG | |
| | ACAGACCCACTGCACTGCAGAGCCCATGGCGGCAGCTGGTATAGCGTG | |
| | AGGGCCAACTACTGGGGCCAAGGCACACAAGTGACAGTCTCGAGCGGC | |
| | GGAGGATCCCAAGTGCAGCTGCAAGAGTCCGGCGGAGGCAGCGTCCAA | |
| | GCCGGAGGATCTCTGAGGCTGAGCTGTACAGTGAGCAGATACACTGCC | |
| | AGCGTGAACTACATGGGCTGGTTCAGACAAGCCCCCGGCAAAGAGAGA | |
| | GAGGGCGTGGCCACAATCTTCACTGGCGCCGGCACAACATACTACGCC | |
| | AACTCCGTCAAGGGAAGGTTCACAATCTCTAGGGACAACGCCAAGAAC | |
| | ACTGCCTATCTGCAGATGAACTCCCTCAAGCCAGAGGACACTGCCATC | |
| | TACTACTGCGCCGTGGATTTCAGAGGCGGACTGCTGTATAGGCCAGCC | |
| | TACGAGTACACTTATAGGGGCCAAGGCACACAAGTGACAGTCTCGTCT | |
| | GCTAGCCACCATCACCATCACCAC | |
| 302 | CAAGTGCAGCTGCAAGAGAGCGGAGGAGGAAGCGTCCAAGCCGGAGGC | 400 |
| | TCTCTGAGACTGAGCTGTGCCGCCAGCGGCTACTCCAACTGCAGCTAC | |
| | GACATGACTTGGTATAGGCAAGCCCCCGGCAAGGAGAGGGAGTTCGTG | |
| | TCCGCCATCCACAGCGACGGCAGCACTAGATACGCCGACAGCGTGAAG | |
| | GGAAGGTTCTTCATCAGCCAAGATAACGCCAAGAACACAGTGTATCTG | |
| | CAGATGAACTCCCTCAAGCCAGAGGACACTGCCATGTACTACTGCAAG | |
| | ACAGACCCACTGCACTGCAGAGCCCATGGCGGCAGCTGGTATAGCGTG | |
| | AGGGCCAACTACTGGGGCCAAGGCACACAAGTGACAGTCTCGAGCGGC | |
| | GGAGGATCCCAAGTGCAGCTGCAAGAGAGCGGAGGAGGCAGCGTCGAA | |
| | GCTGGAGGATCTCTGAGGCTGAGCTGTGCTGCCAGCGGCTACACTCAC | |
| | AGCAGCTACTGTATGGGCTGGTTCAGACAAGCCCCCGGCAAGGAGAGG | |
| | GAAGGCGTGGCTGCCATCGACGTGGATGGCAGCACTACTTACGCCGAC | |
| | AGCGTGAAGGGAAGGTTCACTATCAGCAAGGACAACGCCAAGAACACA | |
| | CTCTATCTGCAGATGAACAGCCTCAAGCCAGAGGACACTGGCATGTAC | |
| | TACTGCGCCGCCGAGTTCGCCGATTGCAGCAGCAACTACTTTCTGCCT | |
| | CCCGGCGCCGTCAGATATTGGGGCCAAGGCACTCAAGTGACAGTCTCG | |
| | TCTGCTAGCCACCATCACCATCACCAC | |
| 303 | CAAGTGCAGCTGCAAGAGAGCGGAGGAGGAAGCGTCCAAGCCGGAGGC | 401 |
| | TCTCTGAGACTGAGCTGTGCCGCCAGCGGCTACTCCAACTGCAGCTAC | |
| | GACATGACTTGGTATAGGCAAGCCCCCGGCAAGGAGAGGGAGTTCGTG | |
| | TCCGCCATCCACAGCGACGGCAGCACTAGATACGCCGACAGCGTGAAG | |
| | GGAAGGTTCTTCATCAGCCAAGATAACGCCAAGAACACAGTGTATCTG | |
| | CAGATGAACTCCCTCAAGCCAGAGGACACTGCCATGTACTACTGCAAG | |
| | ACAGACCCACTGCACTGCAGAGCCCATGGCGGCAGCTGGTATAGCGTG | |
| | AGGGCCAACTACTGGGGCCAAGGCACACAAGTGACAGTCTCGAGCGGC | |
| | GGAGGATCCCAAGTGCAGCTGCAAGAGAGCGGAGGAGGAAGCGTCCAA | |
| | GCCGGAGGATCTCTGAGACTGAGCTGCGCCGCTAGTGGCTACTCCTAC | |
| | AGCAGCTACTGCATGGGCTGGTTTAGGCAAGCCCCCGGCAAGGAGAGA | |
| | GAAGGCGTGGCCACTATCGACAGCGACGGCATGACAAGGTACGCCGAC | |
| | AGCGTGAAGGGAAGGTTCACAATCAGCAAGGACAACGCCAAGAACACA | |
| | CTGTATCTGCAGATGAACTCTCTGAAGCCAGAGGACACTGCCATGTAC | |
| | TACTGTGCCGCTCCTCTGTACGACTGTGATAGCGGCGCTGTGGGCAGA | |
| | AATCCACCTTATTGGGGCCAAGGCACTCAAGTGACAGTCTCGTCTGCT | |
| | AGCCACCATCACCATCACCAC | |
| 304 | CAAGTGCAGCTGCAAGAGAGCGGAGGAGGAAGCGTCCAAGCCGGAGGC | 402 |
| | TCTCTGAGACTGAGCTGTGCCGCCAGCGGCTACTCCAACTGCAGCTAC | |
| | GACATGACTTGGTATAGGCAAGCCCCCGGCAAGGAGAGGGAGTTCGTG | |
| | TCCGCCATCCACAGCGACGGCAGCACTAGATACGCCGACAGCGTGAAG | |
| | GGAAGGTTCTTCATCAGCCAAGATAACGCCAAGAACACAGTGTATCTG | |
| | CAGATGAACTCCCTCAAGCCAGAGGACACTGCCATGTACTACTGCAAG | |
| | ACAGACCCACTGCACTGCAGAGCCCATGGCGGCAGCTGGTATAGCGTG | |
| | AGGGCCAACTACTGGGGCCAAGGCACACAAGTGACAGTCTCGAGCGGC | |
| | GGAGGATCCCAAGTGCAGCTGCAAGAGAGCGGAGGCGGCAGCGTGCAG | |
| | ACTGGAGGCTCTCTGAGACTGAGCTGTGCTGCCAGCGGCTACACTTAT | |
| | CTGAGGGGCTGTATGGGCTGGTTTAGGCAAGCCCCCGGCAAGGAGAGA | |
| | GAGGGCGTGGCCGTCATGGATGTGGTGGGCGATAGGAGAAGCTACATC | |
| | GACAGCGTGAAGGGAAGGTTCACAATCTCTAGGGACAATGCCGCCAAC | |
| | AGCGTCTATCTGCAGATGGACAATCTGAAGCCAGAGGACACAGCCATG | |
| | TACTACTGCACTGCCGGCCCTAACTGTGTGGGCTGGAGAAGCGGACTG | |
| | GATTACTGGGGCCAAGGCACACAAGTGACAGTCTCGTCTGCTAGCCAC | |
| | CATCACCATCACCAC | |
| 305 | CAAGTGCAGCTGCAAGAGAGCGGCGGAGGAAGCGTGCAAGCTGGAGGC | 403 |
| | TCTCTGAGGCTGAGCTGTGCTGCCAGCGGCTACACTTATAGCAGCGGC | |
| | TGTATGGGCTGGTTCAGACAAGCCCCCGGCAAGGAAAGGGAAGCCGTG | |
| | GCCGCCATCAATTCCGATGGCAGCACAAGCTACGCCGACAGCGTGAAG | |
| | GGAAGGTTCACAATCAGCAAGGACAACGCCAAGAACACACTCTATCTG | |
| | CAGATGAACTCTCTGAAGCCAGAGGACACAGCCATGTACTACTGCGCC | |
| | GCTGAGCCTTACTGTAGCGGCGGCTACCCAAGATGGAGCGTCGCTGAG | |
| | TTCGGCTACTGGGGCCAAGGCACACAAGTGACTGTCTCGAGCGGCGGA | |
| | GGATCCCAAGTGCAGCTGCAAGAGAGCGGAGGAGGAAGCGTGCAAGCC | |
| | GGAGGCTCTCTGAGACTGAGCTGTGCCGCCTCTAGGTATCTGTACAGC | |
| | ATCGACTACATGGCTTGGTTCAGACAGAGCCCCGGCAAGGAGAGGGAG | |
| | CCAGTGGCTGTCATCTACACTGCCTCCGGCGCCACATTCTATCCAGAT | |
| | AGCGTGAAGGGAAGGTTCACTATCAGCCAAGATAACGCCAAGATGACA | |
| | GTGTATCTGCAGATGAACTCTCTGAAGAGCGAGGACACTGCCATGTAC | |
| | TACTGTGCCGCCGTGAGGAAGACAGATAGCTACCTCTTCGACGCCCAG | |
| | AGCTTCACATACTGGGGCCAAGGCACACAAGTGACAGTCTCGTCTGCT | |
| | AGCCACCATCACCATCACCAC | |
| 306 | CAAGTGCAGCTGCAAGAGAGCGGCGGAGGAAGCGTGCAAGCTGGAGGC | 404 |
| | TCTCTGAGGCTGAGCTGTGCTGCCAGCGGCTACACTTATAGCAGCGGC | |
| | TGTATGGGCTGGTTCAGACAAGCCCCCGGCAAGGAAAGGGAAGCCGTG | |
| | GCCGCCATCAATTCCGATGGCAGCACAAGCTACGCCGACAGCGTGAAG | |
| | GGAAGGTTCACAATCAGCAAGGACAACGCCAAGAACACACTCTATCTG | |
| | CAGATGAACTCTCTGAAGCCAGAGGACACAGCCATGTACTACTGCGCC | |
| | GCTGAGCCTTACTGTAGCGGCGGCTACCCAAGATGGAGCGTCGCTGAG | |
| | TTCGGCTACTGGGGCCAAGGCACACAAGTGACTGTCTCGAGCGGCGGA | |
| | GGATCCCAAGTGCAGCTGCAAGAGAGCGGAGGAGGAAGCGTGCAAGCC | |
| | GGAGGCTCTCTGAGGCTGAGCTGTGCCGCCTCTAGGTTCACATACAGC | |
| | AGCTACTGCATGGGCTGGTTCAGACAAGCCCCCGGCAAAGAGAGAGAA | |
| | GGCGTGGCCAGCATCGATAGCGATGGCTCCACTAGCTACACTGACAGC | |
| | GTGAAGGGAAGGTTCACTATCAGCAAGGACAACGCCAAGAACACTCTG | |
| | TATCTGCAGATGAACTCTCTGAAGCCAGAGGACACAGCCATGTACTAC | |
| | TGTGCCCTCGATCTGATGAGCACAGTGGTGCCCGGCTTCTGTGGCTTT | |
| | CTGCTGAGCGCTGGCATGGATTACTGGGGCAAGGGCACTCAAGTGACT | |
| | GTCTCGTCTGCTAGCCACCATCACCATCACCAC | |
| 307 | CAAGTGCAGCTGCAAGAGAGCGGCGGAGGAAGCGTGCAAGCTGGAGGC | 405 |
| | TCTCTGAGGCTGAGCTGTGCTGCCAGCGGCTACACTTATAGCAGCGGC | |
| | TGTATGGGCTGGTTCAGACAAGCCCCCGGCAAGGAAAGGGAAGCCGTG | |
| | GCCGCCATCAATTCCGATGGCAGCACAAGCTACGCCGACAGCGTGAAG | |
| | GGAAGGTTCACAATCAGCAAGGACAACGCCAAGAACACACTCTATCTG | |
| | CAGATGAACTCTCTGAAGCCAGAGGACACAGCCATGTACTACTGCGCC | |
| | GCTGAGCCTTACTGTAGCGGCGGCTACCCAAGATGGAGCGTCGCTGAG | |
| | TTCGGCTACTGGGGCCAAGGCACACAAGTGACTGTCTCGAGCGGCGGA | |
| | GGATCCCAAGTGCAGCTGCAAGAGTCCGGAGGAGGCAGCGTCCAAGCC | |
| | GGAGGCTCTCTGAGGCTGAGCTGTGCTGCCAGCGGCTACACTTACAGC | |
| | ATGTACTGCATGGGCTGGTTCAGACAAGCCCCCGGCAAGGAAAGAGAG | |
| | GGCGTGGCCCAGATCAATAGCGATGGCAGCACAAGCTACGCCGACAGC | |
| | GTGAAGGGAAGGTTCACTATCTCCAAGGACAACGCCAAGAACACTCTG | |
| | TATCTGCAGATGAACTCTCTGAAGCCAGAGGACACTGCCATGTACTAC | |
| | TGCGCTGCCGATTCTAGGGTGTACGGCGGCAGCTGGTATGAGAGGCTC | |
| | TGCGGCCCTTACACATACGAGTACAACTACTGGGGCCAAGGCACACAA | |
| | GTGACTGTCTCGTCTGCTAGCCACCATCACCATCACCAC | |
| 308 | CAAGTGCAGCTGCAAGAGAGCGGCGGAGGAAGCGTGCAAGCTGGAGGC | 406 |
| | TCTCTGAGGCTGAGCTGTGCTGCCAGCGGCTACACTTATAGCAGCGGC | |
| | TGTATGGGCTGGTTCAGACAAGCCCCCGGCAAGGAAAGGGAAGCCGTG | |
| | GCCGCCATCAATTCCGATGGCAGCACAAGCTACGCCGACAGCGTGAAG | |
| | GGAAGGTTCACAATCAGCAAGGACAACGCCAAGAACACACTCTATCTG | |
| | CAGATGAACTCTCTGAAGCCAGAGGACACAGCCATGTACTACTGCGCC | |
| | GCTGAGCCTTACTGTAGCGGCGGCTACCCAAGATGGAGCGTCGCTGAG | |
| | TTCGGCTACTGGGGCCAAGGCACACAAGTGACTGTCTCGAGCGGCGGA | |
| | GGATCCCAAGTGCAGCTGCAAGAGAGCGGCGGAGGAAGCGTGCAAGCC | |
| | GGAGGATCTCTGAGACTGAGCTGCGCTGTGAGCGGCTACGCCTACTCC | |
| | ACATACTGCATGGGCTGGTTTAGGCAAGCCCCCGGCAAAGAGAGAGAG | |
| | GGCGTGGCTGCTATCGATAGCGGCGGCAGCACAAGCTACGCCGATAGC | |
| | GTGAAGGGAAGGTTCACAATCAGCAAGGACAACGCCAAGAACACACTG | |
| | TATCTGAGGATGAACTCTCTGAAGCCAGAGGACACAGCCATGTACTAC | |
| | TGTGCTGCTGTGCCTCCTCCTCCAGATGGCGGCAGCTGTCTGTTTCTG | |
| | GGACCAGAGATCAAGGTCAGCAAGGCCGATTTTAGGTACTGGGGCCAA | |
| | GGCACACAAGTGACAGTCTCGTCTGCTAGCCACCATCACCATCACCAC | |
| 309 | CAAGTGCAGCTGCAAGAGAGCGGCGGAGGAAGCGTGCAAGCTGGAGGC | 407 |
| | TCTCTGAGGCTGAGCTGTGCTGCCAGCGGCTACACTTATAGCAGCGGC | |
| | TGTATGGGCTGGTTCAGACAAGCCCCCGGCAAGGAAAGGGAAGCCGTG | |
| | GCCGCCATCAATTCCGATGGCAGCACAAGCTACGCCGACAGCGTGAAG | |
| | GGAAGGTTCACAATCAGCAAGGACAACGCCAAGAACACACTCTATCTG | |
| | CAGATGAACTCTCTGAAGCCAGAGGACACAGCCATGTACTACTGCGCC | |
| | GCTGAGCCTTACTGTAGCGGCGGCTACCCAAGATGGAGCGTCGCTGAG | |
| | TTCGGCTACTGGGGCCAAGGCACACAAGTGACTGTCTCGAGCGGCGGA | |
| | GGATCCCAAGTGCAGCTGCAAGAGAGCGGAGGAGGAAGCGTGCAAGCC | |
| | GGAGGCTCTCTGAGGCTGAGCTGTACAGTGTCCGGCTACACTTACAGC | |
| | TCCAATTGCATGGGCTGGTTTAGGCAAGCCCCCGGCAAGGAAAGAGAG | |
| | GGCGTGGCCACTATCTACACTGGCGGCGGCAACACATACTACGCCGAT | |
| | AGCGTGAAGGGAAGGTTCACTATCAGCCAAGATAACGCCAAGAACACA | |
| | GTGTATCTGCAGATGAACAATCTGAAGCCAGAGGACACTGCCATGTAC | |
| | TACTGTGCTGCTGAGCCACTGTCTAGGGTGTACGGCGGCAGCTGCCCA | |
| | ACTCCTACATTCGACTACTGGGGCCAAGGCACACAAGTGACTGTCTCG | |
| | TCTGCTAGCCACCATCACCATCACCAC | |
| 310 | CAAGTGCAGCTGCAAGAGAGCGGCGGAGGAAGCGTGCAAGCTGGAGGC | 408 |
| | TCTCTGAGGCTGAGCTGTGCTGCCAGCGGCTACACTTATAGCAGCGGC | |
| | TGTATGGGCTGGTTCAGACAAGCCCCCGGCAAGGAAAGGGAAGCCGTG | |
| | GCCGCCATCAATTCCGATGGCAGCACAAGCTACGCCGACAGCGTGAAG | |
| | GGAAGGTTCACAATCAGCAAGGACAACGCCAAGAACACACTCTATCTG | |
| | CAGATGAACTCTCTGAAGCCAGAGGACACAGCCATGTACTACTGCGCC | |
| | GCTGAGCCTTACTGTAGCGGCGGCTACCCAAGATGGAGCGTCGCTGAG | |
| | TTCGGCTACTGGGGCCAAGGCACACAAGTGACTGTCTCGAGCGGCGGA | |
| | GGATCCCAAGTGCAGCTGCAAGAGAGCGGAGGAGGAAGCGTCCAAGCC | |
| | GGAGGCTCTCTGAGGCTGAGCTGTGGAGCCAGCGGCTACACTTACAGC | |
| | AGCTACTGTATGGGCTGGTTTAGGCAAGTGCCCGGCAAGGAGAGAGAG | |
| | GGCGTGGCCGTGATCGATTCCGATGGCAGCACAAGCTACGCTGACAGC | |
| | GTGAAGGGAAGGTTCACAATCAGCAAGGACAACGGCAAGAACACACTC | |
| | TATCTGCAGATGAACAGCCTCAAGCCAGAGGACACAGCCATGTACTAC | |
| | TGCGCCGCTGATCTGGGCCACTATAGGCCTCCTTGTGGCGTGCTGTAT | |
| | CTGGGCATGGATTACTGGGGCAAGGGCACACAAGTGACAGTCTCGTCT | |
| | GCTAGCCACCATCACCATCACCAC | |
| 311 | CAAGTGCAGCTGCAAGAGAGCGGCGGAGGAAGCGTGCAAGCTGGAGGC | 409 |
| | TCTCTGAGGCTGAGCTGTGCTGCCAGCGGCTACACTTATAGCAGCGGC | |
| | TGTATGGGCTGGTTCAGACAAGCCCCCGGCAAGGAAAGGGAAGCCGTG | |
| | GCCGCCATCAATTCCGATGGCAGCACAAGCTACGCCGACAGCGTGAAG | |
| | GGAAGGTTCACAATCAGCAAGGACAACGCCAAGAACACACTCTATCTG | |
| | CAGATGAACTCTCTGAAGCCAGAGGACACAGCCATGTACTACTGCGCC | |
| | GCTGAGCCTTACTGTAGCGGCGGCTACCCAAGATGGAGCGTCGCTGAG | |
| | TTCGGCTACTGGGGCCAAGGCACACAAGTGACTGTCTCGAGCGGCGGA | |
| | GGATCCCAAGTGCAGCTGCAAGAGAGCGGAGGAGGAAGCGTCCAAGCC | |
| | GGAGGCTCTCTGAGACTGAGCTGTGCCGCCAGCGGCTACTCCAACTGC | |
| | AGCTACGACATGACTTGGTATAGGCAAGCCCCCGGCAAGGAGAGGGAG | |
| | TTCGTGTCCGCCATCCACAGCGACGGCAGCACTAGATACGCCGACAGC | |
| | GTGAAGGGAAGGTTCTTCATCAGCCAAGATAACGCCAAGAACACAGTG | |
| | TATCTGCAGATGAACTCCCTCAAGCCAGAGGACACTGCCATGTACTAC | |
| | TGCAAGACAGACCCACTGCACTGCAGAGCCCATGGCGGCAGCTGGTAT | |
| | AGCGTGAGGGCCAACTACTGGGGCCAAGGCACACAAGTGACAGTCTCG | |
| | TCTGCTAGCCACCATCACCATCACCAC | |
| 312 | CAAGTGCAGCTGCAAGAGAGCGGAGGAGGCAGCGTCCAAGCCGGAGGC | 410 |
| | TCTCTGAGGCTGAGCTGTGCTGCCAGCGGCTACACTTACAGCAGCTAC | |
| | TGCATGGGCTGGTTCAGACAAGCCCCCGGCAAGGAGAGAGAGGGCGTG | |
| | GCTCACATCGACAGCGACGGCTCCACAAGCTACGCCGATAGCGTGAAG | |
| | GGAAGGTTCACAATCTCCAAGGACAACGCCAAGAACACTCTGTACCTC | |
| | CAGATGAACTCTCTGAAGCCAGAGGACACTGCCATGTACTACTGTGCC | |
| | GCCGATCCAATTCCCGGCCCCGGCTACTGCGATGGCGGCCCTAACAAG | |
| | TACTGGGGCCAAGGCACACAAGTGACTGTCTCGAGCGGCGGAGGATCC | |
| | CAAGTGCAGCTGCAAGAGAGCGGAGGAGGAAGCGTGCAAGCCGGAGGC | |
| | TCTCTGAGACTGAGCTGTGCCGCCTCTAGGTATCTGTACAGCATCGAC | |
| | TACATGGCTTGGTTCAGACAGAGCCCCGGCAAGGAGAGGGAGCCAGTG | |
| | GCTGTCATCTACACTGCCTCCGGCGCCACATTCTATCCAGATAGCGTG | |
| | AAGGGAAGGTTCACTATCAGCCAAGATAACGCCAAGATGACAGTGTAT | |
| | CTGCAGATGAACTCTCTGAAGAGCGAGGACACTGCCATGTACTACTGT | |
| | GCCGCCGTGAGGAAGACAGATAGCTACCTCTTCGACGCCCAGAGCTTC | |
| | ACATACTGGGGCCAAGGCACACAAGTGACAGTCTCGTCTGCTAGCCAC | |
| | CATCACCATCACCAC | |
| 313 | CAAGTGCAGCTGCAAGAGAGCGGAGGAGGCAGCGTCCAAGCCGGAGGC | 411 |
| | TCTCTGAGGCTGAGCTGTGCTGCCAGCGGCTACACTTACAGCAGCTAC | |
| | TGCATGGGCTGGTTCAGACAAGCCCCCGGCAAGGAGAGAGAGGGCGTG | |
| | GCTCACATCGACAGCGACGGCTCCACAAGCTACGCCGATAGCGTGAAG | |
| | GGAAGGTTCACAATCTCCAAGGACAACGCCAAGAACACTCTGTACCTC | |
| | CAGATGAACTCTCTGAAGCCAGAGGACACTGCCATGTACTACTGTGCC | |
| | GCCGATCCAATTCCCGGCCCCGGCTACTGCGATGGCGGCCCTAACAAG | |
| | TACTGGGGCCAAGGCACACAAGTGACTGTCTCGAGCGGCGGAGGATCC | |
| | CAAGTGCAGCTGCAAGAGAGCGGAGGAGGAAGCGTGCAAGCCGGAGGC | |
| | TCTCTGAGGCTGAGCTGTGCCGCCTCTAGGTTCACATACAGCAGCTAC | |
| | TGCATGGGCTGGTTCAGACAAGCCCCCGGCAAAGAGAGAGAAGGCGTG | |
| | GCCAGCATCGATAGCGATGGCTCCACTAGCTACACTGACAGCGTGAAG | |
| | GGAAGGTTCACTATCAGCAAGGACAACGCCAAGAACACTCTGTATCTG | |
| | CAGATGAACTCTCTGAAGCCAGAGGACACAGCCATGTACTACTGTGCC | |
| | CTCGATCTGATGAGCACAGTGGTGCCCGGCTTCTGTGGCTTTCTGCTG | |
| | AGCGCTGGCATGGATTACTGGGGCAAGGGCACTCAAGTGACTGTCTCG | |
| | TCTGCTAGCCACCATCACCATCACCAC | |
| 314 | CAAGTGCAGCTGCAAGAGAGCGGAGGAGGCAGCGTCCAAGCCGGAGGC | 412 |
| | TCTCTGAGGCTGAGCTGTGCTGCCAGCGGCTACACTTACAGCAGCTAC | |
| | TGCATGGGCTGGTTCAGACAAGCCCCCGGCAAGGAGAGAGAGGGCGTG | |
| | GCTCACATCGACAGCGACGGCTCCACAAGCTACGCCGATAGCGTGAAG | |
| | GGAAGGTTCACAATCTCCAAGGACAACGCCAAGAACACTCTGTACCTC | |
| | CAGATGAACTCTCTGAAGCCAGAGGACACTGCCATGTACTACTGTGCC | |
| | GCCGATCCAATTCCCGGCCCCGGCTACTGCGATGGCGGCCCTAACAAG | |
| | TACTGGGGCCAAGGCACACAAGTGACTGTCTCGAGCGGCGGAGGATCC | |
| | CAAGTGCAGCTGCAAGAGTCCGGAGGAGGCAGCGTCCAAGCCGGAGGC | |
| | TCTCTGAGGCTGAGCTGTGCTGCCAGCGGCTACACTTACAGCATGTAC | |
| | TGCATGGGCTGGTTCAGACAAGCCCCCGGCAAGGAAAGAGAGGGCGTG | |
| | GCCCAGATCAATAGCGATGGCAGCACAAGCTACGCCGACAGCGTGAAG | |
| | GGAAGGTTCACTATCTCCAAGGACAACGCCAAGAACACTCTGTATCTG | |
| | CAGATGAACTCTCTGAAGCCAGAGGACACTGCCATGTACTACTGCGCT | |
| | GCCGATTCTAGGGTGTACGGCGGCAGCTGGTATGAGAGGCTCTGCGGC | |
| | CCTTACACATACGAGTACAACTACTGGGGCCAAGGCACACAAGTGACT | |
| | GTCTCGTCTGCTAGCCACCATCACCATCACCAC | |
| 315 | CAAGTGCAGCTGCAAGAGAGCGGAGGAGGCAGCGTCCAAGCCGGAGGC | 413 |
| | TCTCTGAGGCTGAGCTGTGCTGCCAGCGGCTACACTTACAGCAGCTAC | |
| | TGCATGGGCTGGTTCAGACAAGCCCCCGGCAAGGAGAGAGAGGGCGTG | |
| | GCTCACATCGACAGCGACGGCTCCACAAGCTACGCCGATAGCGTGAAG | |
| | GGAAGGTTCACAATCTCCAAGGACAACGCCAAGAACACTCTGTACCTC | |
| | CAGATGAACTCTCTGAAGCCAGAGGACACTGCCATGTACTACTGTGCC | |
| | GCCGATCCAATTCCCGGCCCCGGCTACTGCGATGGCGGCCCTAACAAG | |
| | TACTGGGGCCAAGGCACACAAGTGACTGTCTCGAGCGGCGGAGGATCC | |
| | CAAGTGCAGCTGCAAGAGAGCGGCGGAGGAAGCGTGCAAGCCGGAGGA | |
| | TCTCTGAGACTGAGCTGCGCTGTGAGCGGCTACGCCTACTCCACATAC | |
| | TGCATGGGCTGGTTTAGGCAAGCCCCCGGCAAAGAGAGAGAGGGCGTG | |
| | GCTGCTATCGATAGCGGCGGCAGCACAAGCTACGCCGATAGCGTGAAG | |
| | GGAAGGTTCACAATCAGCAAGGACAACGCCAAGAACACACTGTATCTG | |
| | AGGATGAACTCTCTGAAGCCAGAGGACACAGCCATGTACTACTGTGCT | |
| | GCTGTGCCTCCTCCTCCAGATGGCGGCAGCTGTCTGTTTCTGGGACCA | |
| | GAGATCAAGGTCAGCAAGGCCGATTTTAGGTACTGGGGCCAAGGCACA | |
| | CAAGTGACAGTCTCGTCTGCTAGCCACCATCACCATCACCAC | |
| 316 | CAAGTGCAGCTGCAAGAGAGCGGAGGAGGCAGCGTCCAAGCCGGAGGC | 414 |
| | TCTCTGAGGCTGAGCTGTGCTGCCAGCGGCTACACTTACAGCAGCTAC | |
| | TGCATGGGCTGGTTCAGACAAGCCCCCGGCAAGGAGAGAGAGGGCGTG | |
| | GCTCACATCGACAGCGACGGCTCCACAAGCTACGCCGATAGCGTGAAG | |
| | GGAAGGTTCACAATCTCCAAGGACAACGCCAAGAACACTCTGTACCTC | |
| | CAGATGAACTCTCTGAAGCCAGAGGACACTGCCATGTACTACTGTGCC | |
| | GCCGATCCAATTCCCGGCCCCGGCTACTGCGATGGCGGCCCTAACAAG | |
| | TACTGGGGCCAAGGCACACAAGTGACTGTCTCGAGCGGCGGAGGATCC | |
| | CAAGTGCAGCTGCAAGAGAGCGGAGGAGGAAGCGTGCAAGCCGGAGGC | |
| | TCTCTGAGGCTGAGCTGTACAGTGTCCGGCTACACTTACAGCTCCAAT | |
| | TGCATGGGCTGGTTTAGGCAAGCCCCCGGCAAGGAAAGAGAGGGCGTG | |
| | GCCACTATCTACACTGGCGGCGGCAACACATACTACGCCGATAGCGTG | |
| | AAGGGAAGGTTCACTATCAGCCAAGATAACGCCAAGAACACAGTGTAT | |
| | CTGCAGATGAACAATCTGAAGCCAGAGGACACTGCCATGTACTACTGT | |
| | GCTGCTGAGCCACTGTCTAGGGTGTACGGCGGCAGCTGCCCAACTCCT | |
| | ACATTCGACTACTGGGGCCAAGGCACACAAGTGACTGTCTCGTCTGCT | |
| | AGCCACCATCACCATCACCAC | |
| 317 | CAAGTGCAGCTGCAAGAGAGCGGAGGAGGCAGCGTCCAAGCCGGAGGC | 415 |
| | TCTCTGAGGCTGAGCTGTGCTGCCAGCGGCTACACTTACAGCAGCTAC | |
| | TGCATGGGCTGGTTCAGACAAGCCCCCGGCAAGGAGAGAGAGGGCGTG | |
| | GCTCACATCGACAGCGACGGCTCCACAAGCTACGCCGATAGCGTGAAG | |
| | GGAAGGTTCACAATCTCCAAGGACAACGCCAAGAACACTCTGTACCTC | |
| | CAGATGAACTCTCTGAAGCCAGAGGACACTGCCATGTACTACTGTGCC | |
| | GCCGATCCAATTCCCGGCCCCGGCTACTGCGATGGCGGCCCTAACAAG | |
| | TACTGGGGCCAAGGCACACAAGTGACTGTCTCGAGCGGCGGAGGATCC | |
| | CAAGTGCAGCTGCAAGAGAGCGGAGGAGGAAGCGTCCAAGCCGGAGGC | |
| | TCTCTGAGGCTGAGCTGTGGAGCCAGCGGCTACACTTACAGCAGCTAC | |
| | TGTATGGGCTGGTTTAGGCAAGTGCCCGGCAAGGAGAGAGAGGGCGTG | |
| | GCCGTGATCGATTCCGATGGCAGCACAAGCTACGCTGACAGCGTGAAG | |
| | GGAAGGTTCACAATCAGCAAGGACAACGGCAAGAACACACTCTATCTG | |
| | CAGATGAACAGCCTCAAGCCAGAGGACACAGCCATGTACTACTGCGCC | |
| | GCTGATCTGGGCCACTATAGGCCTCCTTGTGGCGTGCTGTATCTGGGC | |
| | ATGGATTACTGGGGCAAGGGCACACAAGTGACAGTCTCGTCTGCTAGC | |
| | CACCATCACCATCACCAC | |
| 318 | CAAGTGCAGCTGCAAGAGAGCGGAGGAGGCAGCGTCCAAGCCGGAGGC | 416 |
| | TCTCTGAGGCTGAGCTGTGCTGCCAGCGGCTACACTTACAGCAGCTAC | |
| | TGCATGGGCTGGTTCAGACAAGCCCCCGGCAAGGAGAGAGAGGGCGTG | |
| | GCTCACATCGACAGCGACGGCTCCACAAGCTACGCCGATAGCGTGAAG | |
| | GGAAGGTTCACAATCTCCAAGGACAACGCCAAGAACACTCTGTACCTC | |
| | CAGATGAACTCTCTGAAGCCAGAGGACACTGCCATGTACTACTGTGCC | |
| | GCCGATCCAATTCCCGGCCCCGGCTACTGCGATGGCGGCCCTAACAAG | |
| | TACTGGGGCCAAGGCACACAAGTGACTGTCTCGAGCGGCGGAGGATCC | |
| | CAAGTGCAGCTGCAAGAGAGCGGAGGAGGAAGCGTCCAAGCCGGAGGC | |
| | TCTCTGAGACTGAGCTGTGCCGCCAGCGGCTACTCCAACTGCAGCTAC | |
| | GACATGACTTGGTATAGGCAAGCCCCCGGCAAGGAGAGGGAGTTCGTG | |
| | TCCGCCATCCACAGCGACGGCAGCACTAGATACGCCGACAGCGTGAAG | |
| | GGAAGGTTCTTCATCAGCCAAGATAACGCCAAGAACACAGTGTATCTG | |
| | CAGATGAACTCCCTCAAGCCAGAGGACACTGCCATGTACTACTGCAAG | |
| | ACAGACCCACTGCACTGCAGAGCCCATGGCGGCAGCTGGTATAGCGTG | |
| | AGGGCCAACTACTGGGGCCAAGGCACACAAGTGACAGTCTCGTCTGCT | |
| | AGCCACCATCACCATCACCAC | |
| 319 | CAAGTGCAGCTGCAAGAGTCCGGAGGAGGAAGCGTGCAAGCCGGCGGA | 417 |
| | TCTCTGAGACTGAGCTGTGCCGCCTCTAGGTACACTTACAACAGCTAC | |
| | TGCATGGGCTGGTTCAGACAAGCCCCCGGCAAGGAAAGAGAGGGCGTG | |
| | GCCACTATCGATAGCGACGGCATGACTAGGTACGCTGATAGCGTCAAG | |
| | GGAAGGTTCACAATCTCCAAGGACAATGCTAAGAACACTCTGTACCTC | |
| | CAGATGAACTCTCTGAAGCCAGAGGACACAGCCATGTACTACTGCGCT | |
| | GCCGATGCCGACTGCACTATCGCCGCCATGACTACTAATCCTCTGGGC | |
| | CAAGGCACACAAGTGACTGTCTCGAGCGGCGGAGGATCCCAAGTGCAG | |
| | CTGCAAGAGAGCGGAGGAGGAAGCGTGCAAGCCGGAGGCTCTCTGAGA | |
| | CTGAGCTGTGCCGCCTCTAGGTATCTGTACAGCATCGACTACATGGCT | |
| | TGGTTCAGACAGAGCCCCGGCAAGGAGAGGGAGCCAGTGGCTGTCATC | |
| | TACACTGCCTCCGGCGCCACATTCTATCCAGATAGCGTGAAGGGAAGG | |
| | TTCACTATCAGCCAAGATAACGCCAAGATGACAGTGTATCTGCAGATG | |
| | AACTCTCTGAAGAGCGAGGACACTGCCATGTACTACTGTGCCGCCGTG | |
| | AGGAAGACAGATAGCTACCTCTTCGACGCCCAGAGCTTCACATACTGG | |
| | GGCCAAGGCACACAAGTGACAGTCTCGTCTGCTAGCCACCATCACCAT | |
| | CACCAC | |
| 320 | CAAGTGCAGCTGCAAGAGTCCGGAGGAGGAAGCGTGCAAGCCGGCGGA | 418 |
| | TCTCTGAGACTGAGCTGTGCCGCCTCTAGGTACACTTACAACAGCTAC | |
| | TGCATGGGCTGGTTCAGACAAGCCCCCGGCAAGGAAAGAGAGGGCGTG | |
| | GCCACTATCGATAGCGACGGCATGACTAGGTACGCTGATAGCGTCAAG | |
| | GGAAGGTTCACAATCTCCAAGGACAATGCTAAGAACACTCTGTACCTC | |
| | CAGATGAACTCTCTGAAGCCAGAGGACACAGCCATGTACTACTGCGCT | |
| | GCCGATGCCGACTGCACTATCGCCGCCATGACTACTAATCCTCTGGGC | |
| | CAAGGCACACAAGTGACTGTCTCGAGCGGCGGAGGATCCCAAGTGCAG | |
| | CTGCAAGAGAGCGGAGGAGGAAGCGTGCAAGCCGGAGGCTCTCTGAGG | |
| | CTGAGCTGTGCCGCCTCTAGGTTCACATACAGCAGCTACTGCATGGGC | |
| | TGGTTCAGACAAGCCCCCGGCAAAGAGAGAGAAGGCGTGGCCAGCATC | |
| | GATAGCGATGGCTCCACTAGCTACACTGACAGCGTGAAGGGAAGGTTC | |
| | ACTATCAGCAAGGACAACGCCAAGAACACTCTGTATCTGCAGATGAAC | |
| | TCTCTGAAGCCAGAGGACACAGCCATGTACTACTGTGCCCTCGATCTG | |
| | ATGAGCACAGTGGTGCCCGGCTTCTGTGGCTTTCTGCTGAGCGCTGGC | |
| | ATGGATTACTGGGGCAAGGGCACTCAAGTGACTGTCTCGTCTGCTAGC | |
| | CACCATCACCATCACCAC | |
| 321 | CAAGTGCAGCTGCAAGAGTCCGGAGGAGGAAGCGTGCAAGCCGGCGGA | 419 |
| | TCTCTGAGACTGAGCTGTGCCGCCTCTAGGTACACTTACAACAGCTAC | |
| | TGCATGGGCTGGTTCAGACAAGCCCCCGGCAAGGAAAGAGAGGGCGTG | |
| | GCCACTATCGATAGCGACGGCATGACTAGGTACGCTGATAGCGTCAAG | |
| | GGAAGGTTCACAATCTCCAAGGACAATGCTAAGAACACTCTGTACCTC | |
| | CAGATGAACTCTCTGAAGCCAGAGGACACAGCCATGTACTACTGCGCT | |
| | GCCGATGCCGACTGCACTATCGCCGCCATGACTACTAATCCTCTGGGC | |
| | CAAGGCACACAAGTGACTGTCTCGAGCGGCGGAGGATCCCAAGTGCAG | |
| | CTGCAAGAGTCCGGAGGAGGCAGCGTCCAAGCCGGAGGCTCTCTGAGG | |
| | CTGAGCTGTGCTGCCAGCGGCTACACTTACAGCATGTACTGCATGGGC | |
| | TGGTTCAGACAAGCCCCCGGCAAGGAAAGAGAGGGCGTGGCCCAGATC | |
| | AATAGCGATGGCAGCACAAGCTACGCCGACAGCGTGAAGGGAAGGTTC | |
| | ACTATCTCCAAGGACAACGCCAAGAACACTCTGTATCTGCAGATGAAC | |
| | TCTCTGAAGCCAGAGGACACTGCCATGTACTACTGCGCTGCCGATTCT | |
| | AGGGTGTACGGCGGCAGCTGGTATGAGAGGCTCTGCGGCCCTTACACA | |
| | TACGAGTACAACTACTGGGGCCAAGGCACACAAGTGACTGTCTCGTCT | |
| | GCTAGCCACCATCACCATCACCAC | |
| 322 | CAAGTGCAGCTGCAAGAGTCCGGAGGAGGAAGCGTGCAAGCCGGCGGA | 420 |
| | TCTCTGAGACTGAGCTGTGCCGCCTCTAGGTACACTTACAACAGCTAC | |
| | TGCATGGGCTGGTTCAGACAAGCCCCCGGCAAGGAAAGAGAGGGCGTG | |
| | GCCACTATCGATAGCGACGGCATGACTAGGTACGCTGATAGCGTCAAG | |
| | GGAAGGTTCACAATCTCCAAGGACAATGCTAAGAACACTCTGTACCTC | |
| | CAGATGAACTCTCTGAAGCCAGAGGACACAGCCATGTACTACTGCGCT | |
| | GCCGATGCCGACTGCACTATCGCCGCCATGACTACTAATCCTCTGGGC | |
| | CAAGGCACACAAGTGACTGTCTCGAGCGGCGGAGGATCCCAAGTGCAG | |
| | CTGCAAGAGAGCGGCGGAGGAAGCGTGCAAGCCGGAGGATCTCTGAGA | |
| | CTGAGCTGCGCTGTGAGCGGCTACGCCTACTCCACATACTGCATGGGC | |
| | TGGTTTAGGCAAGCCCCCGGCAAAGAGAGAGAGGGCGTGGCTGCTATC | |
| | GATAGCGGCGGCAGCACAAGCTACGCCGATAGCGTGAAGGGAAGGTTC | |
| | ACAATCAGCAAGGACAACGCCAAGAACACACTGTATCTGAGGATGAAC | |
| | TCTCTGAAGCCAGAGGACACAGCCATGTACTACTGTGCTGCTGTGCCT | |
| | CCTCCTCCAGATGGCGGCAGCTGTCTGTTTCTGGGACCAGAGATCAAG | |
| | GTCAGCAAGGCCGATTTTAGGTACTGGGGCCAAGGCACACAAGTGACA | |
| | GTCTCGTCTGCTAGCCACCATCACCATCACCAC | |
| 323 | CAAGTGCAGCTGCAAGAGTCCGGAGGAGGAAGCGTGCAAGCCGGCGGA | 421 |
| | TCTCTGAGACTGAGCTGTGCCGCCTCTAGGTACACTTACAACAGCTAC | |
| | TGCATGGGCTGGTTCAGACAAGCCCCCGGCAAGGAAAGAGAGGGCGTG | |
| | GCCACTATCGATAGCGACGGCATGACTAGGTACGCTGATAGCGTCAAG | |
| | GGAAGGTTCACAATCTCCAAGGACAATGCTAAGAACACTCTGTACCTC | |
| | CAGATGAACTCTCTGAAGCCAGAGGACACAGCCATGTACTACTGCGCT | |
| | GCCGATGCCGACTGCACTATCGCCGCCATGACTACTAATCCTCTGGGC | |
| | CAAGGCACACAAGTGACTGTCTCGAGCGGCGGAGGATCCCAAGTGCAG | |
| | CTGCAAGAGAGCGGAGGAGGAAGCGTGCAAGCCGGAGGCTCTCTGAGG | |
| | CTGAGCTGTACAGTGTCCGGCTACACTTACAGCTCCAATTGCATGGGC | |
| | TGGTTTAGGCAAGCCCCCGGCAAGGAAAGAGAGGGCGTGGCCACTATC | |
| | TACACTGGCGGCGGCAACACATACTACGCCGATAGCGTGAAGGGAAGG | |
| | TTCACTATCAGCCAAGATAACGCCAAGAACACAGTGTATCTGCAGATG | |
| | AACAATCTGAAGCCAGAGGACACTGCCATGTACTACTGTGCTGCTGAG | |
| | CCACTGTCTAGGGTGTACGGCGGCAGCTGCCCAACTCCTACATTCGAC | |
| | TACTGGGGCCAAGGCACACAAGTGACTGTCTCGTCTGCTAGCCACCAT | |
| | CACCATCACCAC | |
| 324 | CAAGTGCAGCTGCAAGAGTCCGGAGGAGGAAGCGTGCAAGCCGGCGGA | 422 |
| | TCTCTGAGACTGAGCTGTGCCGCCTCTAGGTACACTTACAACAGCTAC | |
| | TGCATGGGCTGGTTCAGACAAGCCCCCGGCAAGGAAAGAGAGGGCGTG | |
| | GCCACTATCGATAGCGACGGCATGACTAGGTACGCTGATAGCGTCAAG | |
| | GGAAGGTTCACAATCTCCAAGGACAATGCTAAGAACACTCTGTACCTC | |
| | CAGATGAACTCTCTGAAGCCAGAGGACACAGCCATGTACTACTGCGCT | |
| | GCCGATGCCGACTGCACTATCGCCGCCATGACTACTAATCCTCTGGGC | |
| | CAAGGCACACAAGTGACTGTCTCGAGCGGCGGAGGATCCCAAGTGCAG | |
| | CTGCAAGAGAGCGGAGGAGGAAGCGTCCAAGCCGGAGGCTCTCTGAGG | |
| | CTGAGCTGTGGAGCCAGCGGCTACACTTACAGCAGCTACTGTATGGGC | |
| | TGGTTTAGGCAAGTGCCCGGCAAGGAGAGAGAGGGCGTGGCCGTGATC | |
| | GATTCCGATGGCAGCACAAGCTACGCTGACAGCGTGAAGGGAAGGTTC | |
| | ACAATCAGCAAGGACAACGGCAAGAACACACTCTATCTGCAGATGAAC | |
| | AGCCTCAAGCCAGAGGACACAGCCATGTACTACTGCGCCGCTGATCTG | |
| | GGCCACTATAGGCCTCCTTGTGGCGTGCTGTATCTGGGCATGGATTAC | |
| | TGGGGCAAGGGCACACAAGTGACAGTCTCGTCTGCTAGCCACCATCAC | |
| | CATCACCAC | |
| 325 | CAAGTGCAGCTGCAAGAGTCCGGAGGAGGAAGCGTGCAAGCCGGCGGA | 423 |
| | TCTCTGAGACTGAGCTGTGCCGCCTCTAGGTACACTTACAACAGCTAC | |
| | TGCATGGGCTGGTTCAGACAAGCCCCCGGCAAGGAAAGAGAGGGCGTG | |
| | GCCACTATCGATAGCGACGGCATGACTAGGTACGCTGATAGCGTCAAG | |
| | GGAAGGTTCACAATCTCCAAGGACAATGCTAAGAACACTCTGTACCTC | |
| | CAGATGAACTCTCTGAAGCCAGAGGACACAGCCATGTACTACTGCGCT | |
| | GCCGATGCCGACTGCACTATCGCCGCCATGACTACTAATCCTCTGGGC | |
| | CAAGGCACACAAGTGACTGTCTCGAGCGGCGGAGGATCCCAAGTGCAG | |
| | CTGCAAGAGAGCGGAGGAGGAAGCGTCCAAGCCGGAGGCTCTCTGAGA | |
| | CTGAGCTGTGCCGCCAGCGGCTACTCCAACTGCAGCTACGACATGACT | |
| | TGGTATAGGCAAGCCCCCGGCAAGGAGAGGGAGTTCGTGTCCGCCATC | |
| | CACAGCGACGGCAGCACTAGATACGCCGACAGCGTGAAGGGAAGGTTC | |
| | TTCATCAGCCAAGATAACGCCAAGAACACAGTGTATCTGCAGATGAAC | |
| | TCCCTCAAGCCAGAGGACACTGCCATGTACTACTGCAAGACAGACCCA | |
| | CTGCACTGCAGAGCCCATGGCGGCAGCTGGTATAGCGTGAGGGCCAAC | |
| | TACTGGGGCCAAGGCACACAAGTGACAGTCTCGTCTGCTAGCCACCAT | |
| | CACCATCACCAC | |
| 326 | CAAGTGCAGCTGCAAGAGTCCGGCGGAGGCAGCGTCCAAGCCGGAGGA | 424 |
| | TCTCTGAGGCTGAGCTGTACAGTGAGCAGATACACTGCCAGCGTGAAC | |
| | TACATGGGCTGGTTCAGACAAGCCCCCGGCAAAGAGAGAGAGGGCGTG | |
| | GCCACAATCTTCACTGGCGCCGGCACAACATACTACGCCAACTCCGTC | |
| | AAGGGAAGGTTCACAATCTCTAGGGACAACGCCAAGAACACTGCCTAT | |
| | CTGCAGATGAACTCCCTCAAGCCAGAGGACACTGCCATCTACTACTGC | |
| | GCCGTGGATTTCAGAGGCGGACTGCTGTATAGGCCAGCCTACGAGTAC | |
| | ACTTATAGGGGCCAAGGCACACAAGTGACAGTCTCGAGCGGCGGAGGA | |
| | TCCCAAGTGCAGCTGCAAGAGAGCGGAGGAGGAAGCGTGCAAGCCGGA | |
| | GGCTCTCTGAGACTGAGCTGTGCCGCCTCTAGGTATCTGTACAGCATC | |
| | GACTACATGGCTTGGTTCAGACAGAGCCCCGGCAAGGAGAGGGAGCCA | |
| | GTGGCTGTCATCTACACTGCCTCCGGCGCCACATTCTATCCAGATAGC | |
| | GTGAAGGGAAGGTTCACTATCAGCCAAGATAACGCCAAGATGACAGTG | |
| | TATCTGCAGATGAACTCTCTGAAGAGCGAGGACACTGCCATGTACTAC | |
| | TGTGCCGCCGTGAGGAAGACAGATAGCTACCTCTTCGACGCCCAGAGC | |
| | TTCACATACTGGGGCCAAGGCACACAAGTGACAGTCTCGTCTGCTAGC | |
| | CACCATCACCATCACCAC | |
| 327 | CAAGTGCAGCTGCAAGAGTCCGGCGGAGGCAGCGTCCAAGCCGGAGGA | 425 |
| | TCTCTGAGGCTGAGCTGTACAGTGAGCAGATACACTGCCAGCGTGAAC | |
| | TACATGGGCTGGTTCAGACAAGCCCCCGGCAAAGAGAGAGAGGGCGTG | |
| | GCCACAATCTTCACTGGCGCCGGCACAACATACTACGCCAACTCCGTC | |
| | AAGGGAAGGTTCACAATCTCTAGGGACAACGCCAAGAACACTGCCTAT | |
| | CTGCAGATGAACTCCCTCAAGCCAGAGGACACTGCCATCTACTACTGC | |
| | GCCGTGGATTTCAGAGGCGGACTGCTGTATAGGCCAGCCTACGAGTAC | |
| | ACTTATAGGGGCCAAGGCACACAAGTGACAGTCTCGAGCGGCGGAGGA | |
| | TCCCAAGTGCAGCTGCAAGAGAGCGGAGGAGGAAGCGTGCAAGCCGGA | |
| | GGCTCTCTGAGGCTGAGCTGTGCCGCCTCTAGGTTCACATACAGCAGC | |
| | TACTGCATGGGCTGGTTCAGACAAGCCCCCGGCAAAGAGAGAGAAGGC | |
| | GTGGCCAGCATCGATAGCGATGGCTCCACTAGCTACACTGACAGCGTG | |
| | AAGGGAAGGTTCACTATCAGCAAGGACAACGCCAAGAACACTCTGTAT | |
| | CTGCAGATGAACTCTCTGAAGCCAGAGGACACAGCCATGTACTACTGT | |
| | GCCCTCGATCTGATGAGCACAGTGGTGCCCGGCTTCTGTGGCTTTCTG | |
| | CTGAGCGCTGGCATGGATTACTGGGGCAAGGGCACTCAAGTGACTGTC | |
| | TCGTCTGCTAGCCACCATCACCATCACCAC | |
| 328 | CAAGTGCAGCTGCAAGAGTCCGGCGGAGGCAGCGTCCAAGCCGGAGGA | 426 |
| | TCTCTGAGGCTGAGCTGTACAGTGAGCAGATACACTGCCAGCGTGAAC | |
| | TACATGGGCTGGTTCAGACAAGCCCCCGGCAAAGAGAGAGAGGGCGTG | |
| | GCCACAATCTTCACTGGCGCCGGCACAACATACTACGCCAACTCCGTC | |
| | AAGGGAAGGTTCACAATCTCTAGGGACAACGCCAAGAACACTGCCTAT | |
| | CTGCAGATGAACTCCCTCAAGCCAGAGGACACTGCCATCTACTACTGC | |
| | GCCGTGGATTTCAGAGGCGGACTGCTGTATAGGCCAGCCTACGAGTAC | |
| | ACTTATAGGGGCCAAGGCACACAAGTGACAGTCTCGAGCGGCGGAGGA | |
| | TCCCAAGTGCAGCTGCAAGAGTCCGGAGGAGGCAGCGTCCAAGCCGGA | |
| | GGCTCTCTGAGGCTGAGCTGTGCTGCCAGCGGCTACACTTACAGCATG | |
| | TACTGCATGGGCTGGTTCAGACAAGCCCCCGGCAAGGAAAGAGAGGGC | |
| | GTGGCCCAGATCAATAGCGATGGCAGCACAAGCTACGCCGACAGCGTG | |
| | AAGGGAAGGTTCACTATCTCCAAGGACAACGCCAAGAACACTCTGTAT | |
| | CTGCAGATGAACTCTCTGAAGCCAGAGGACACTGCCATGTACTACTGC | |
| | GCTGCCGATTCTAGGGTGTACGGCGGCAGCTGGTATGAGAGGCTCTGC | |
| | GGCCCTTACACATACGAGTACAACTACTGGGGCCAAGGCACACAAGTG | |
| | ACTGTCTCGTCTGCTAGCCACCATCACCATCACCAC | |
| 329 | CAAGTGCAGCTGCAAGAGTCCGGCGGAGGCAGCGTCCAAGCCGGAGGA | 427 |
| | TCTCTGAGGCTGAGCTGTACAGTGAGCAGATACACTGCCAGCGTGAAC | |
| | TACATGGGCTGGTTCAGACAAGCCCCCGGCAAAGAGAGAGAGGGCGTG | |
| | GCCACAATCTTCACTGGCGCCGGCACAACATACTACGCCAACTCCGTC | |
| | AAGGGAAGGTTCACAATCTCTAGGGACAACGCCAAGAACACTGCCTAT | |
| | CTGCAGATGAACTCCCTCAAGCCAGAGGACACTGCCATCTACTACTGC | |
| | GCCGTGGATTTCAGAGGCGGACTGCTGTATAGGCCAGCCTACGAGTAC | |
| | ACTTATAGGGGCCAAGGCACACAAGTGACAGTCTCGAGCGGCGGAGGA | |
| | TCCCAAGTGCAGCTGCAAGAGAGCGGCGGAGGAAGCGTGCAAGCCGGA | |
| | GGATCTCTGAGACTGAGCTGCGCTGTGAGCGGCTACGCCTACTCCACA | |
| | TACTGCATGGGCTGGTTTAGGCAAGCCCCCGGCAAAGAGAGAGAGGGC | |
| | GTGGCTGCTATCGATAGCGGCGGCAGCACAAGCTACGCCGATAGCGTG | |
| | AAGGGAAGGTTCACAATCAGCAAGGACAACGCCAAGAACACACTGTAT | |
| | CTGAGGATGAACTCTCTGAAGCCAGAGGACACAGCCATGTACTACTGT | |
| | GCTGCTGTGCCTCCTCCTCCAGATGGCGGCAGCTGTCTGTTTCTGGGA | |
| | CCAGAGATCAAGGTCAGCAAGGCCGATTTTAGGTACTGGGGCCAAGGC | |
| | ACACAAGTGACAGTCTCGTCTGCTAGCCACCATCACCATCACCAC | |
| 330 | CAAGTGCAGCTGCAAGAGTCCGGCGGAGGCAGCGTCCAAGCCGGAGGA | 428 |
| | TCTCTGAGGCTGAGCTGTACAGTGAGCAGATACACTGCCAGCGTGAAC | |
| | TACATGGGCTGGTTCAGACAAGCCCCCGGCAAAGAGAGAGAGGGCGTG | |
| | GCCACAATCTTCACTGGCGCCGGCACAACATACTACGCCAACTCCGTC | |
| | AAGGGAAGGTTCACAATCTCTAGGGACAACGCCAAGAACACTGCCTAT | |
| | CTGCAGATGAACTCCCTCAAGCCAGAGGACACTGCCATCTACTACTGC | |
| | GCCGTGGATTTCAGAGGCGGACTGCTGTATAGGCCAGCCTACGAGTAC | |
| | ACTTATAGGGGCCAAGGCACACAAGTGACAGTCTCGAGCGGCGGAGGA | |
| | TCCCAAGTGCAGCTGCAAGAGAGCGGAGGAGGAAGCGTGCAAGCCGGA | |
| | GGCTCTCTGAGGCTGAGCTGTACAGTGTCCGGCTACACTTACAGCTCC | |
| | AATTGCATGGGCTGGTTTAGGCAAGCCCCCGGCAAGGAAAGAGAGGGC | |
| | GTGGCCACTATCTACACTGGCGGCGGCAACACATACTACGCCGATAGC | |
| | GTGAAGGGAAGGTTCACTATCAGCCAAGATAACGCCAAGAACACAGTG | |
| | TATCTGCAGATGAACAATCTGAAGCCAGAGGACACTGCCATGTACTAC | |
| | TGTGCTGCTGAGCCACTGTCTAGGGTGTACGGCGGCAGCTGCCCAACT | |
| | CCTACATTCGACTACTGGGGCCAAGGCACACAAGTGACTGTCTCGTCT | |
| | GCTAGCCACCATCACCATCACCAC | |
| 331 | CAAGTGCAGCTGCAAGAGTCCGGCGGAGGCAGCGTCCAAGCCGGAGGA | 429 |
| | TCTCTGAGGCTGAGCTGTACAGTGAGCAGATACACTGCCAGCGTGAAC | |
| | TACATGGGCTGGTTCAGACAAGCCCCCGGCAAAGAGAGAGAGGGCGTG | |
| | GCCACAATCTTCACTGGCGCCGGCACAACATACTACGCCAACTCCGTC | |
| | AAGGGAAGGTTCACAATCTCTAGGGACAACGCCAAGAACACTGCCTAT | |
| | CTGCAGATGAACTCCCTCAAGCCAGAGGACACTGCCATCTACTACTGC | |
| | GCCGTGGATTTCAGAGGCGGACTGCTGTATAGGCCAGCCTACGAGTAC | |
| | ACTTATAGGGGCCAAGGCACACAAGTGACAGTCTCGAGCGGCGGAGGA | |
| | TCCCAAGTGCAGCTGCAAGAGAGCGGAGGAGGAAGCGTCCAAGCCGGA | |
| | GGCTCTCTGAGGCTGAGCTGTGGAGCCAGCGGCTACACTTACAGCAGC | |
| | TACTGTATGGGCTGGTTTAGGCAAGTGCCCGGCAAGGAGAGAGAGGGC | |
| | GTGGCCGTGATCGATTCCGATGGCAGCACAAGCTACGCTGACAGCGTG | |
| | AAGGGAAGGTTCACAATCAGCAAGGACAACGGCAAGAACACACTCTAT | |
| | CTGCAGATGAACAGCCTCAAGCCAGAGGACACAGCCATGTACTACTGC | |
| | GCCGCTGATCTGGGCCACTATAGGCCTCCTTGTGGCGTGCTGTATCTG | |
| | GGCATGGATTACTGGGGCAAGGGCACACAAGTGACAGTCTCGTCTGCT | |
| | AGCCACCATCACCATCACCAC | |
| 332 | CAAGTGCAGCTGCAAGAGTCCGGCGGAGGCAGCGTCCAAGCCGGAGGA | 430 |
| | TCTCTGAGGCTGAGCTGTACAGTGAGCAGATACACTGCCAGCGTGAAC | |
| | TACATGGGCTGGTTCAGACAAGCCCCCGGCAAAGAGAGAGAGGGCGTG | |
| | GCCACAATCTTCACTGGCGCCGGCACAACATACTACGCCAACTCCGTC | |
| | AAGGGAAGGTTCACAATCTCTAGGGACAACGCCAAGAACACTGCCTAT | |
| | CTGCAGATGAACTCCCTCAAGCCAGAGGACACTGCCATCTACTACTGC | |
| | GCCGTGGATTTCAGAGGCGGACTGCTGTATAGGCCAGCCTACGAGTAC | |
| | ACTTATAGGGGCCAAGGCACACAAGTGACAGTCTCGAGCGGCGGAGGA | |
| | TCCCAAGTGCAGCTGCAAGAGAGCGGAGGAGGAAGCGTCCAAGCCGGA | |
| | GGCTCTCTGAGACTGAGCTGTGCCGCCAGCGGCTACTCCAACTGCAGC | |
| | TACGACATGACTTGGTATAGGCAAGCCCCCGGCAAGGAGAGGGAGTTC | |
| | GTGTCCGCCATCCACAGCGACGGCAGCACTAGATACGCCGACAGCGTG | |
| | AAGGGAAGGTTCTTCATCAGCCAAGATAACGCCAAGAACACAGTGTAT | |
| | CTGCAGATGAACTCCCTCAAGCCAGAGGACACTGCCATGTACTACTGC | |
| | AAGACAGACCCACTGCACTGCAGAGCCCATGGCGGCAGCTGGTATAGC | |
| | GTGAGGGCCAACTACTGGGGCCAAGGCACACAAGTGACAGTCTCGTCT | |
| | GCTAGCCACCATCACCATCACCAC | |
| 333 | CAAGTGCAGCTGCAAGAGAGCGGAGGAGGCAGCGTCGAAGCTGGAGGA | 431 |
| | TCTCTGAGGCTGAGCTGTGCTGCCAGCGGCTACACTCACAGCAGCTAC | |
| | TGTATGGGCTGGTTCAGACAAGCCCCCGGCAAGGAGAGGGAAGGCGTG | |
| | GCTGCCATCGACGTGGATGGCAGCACTACTTACGCCGACAGCGTGAAG | |
| | GGAAGGTTCACTATCAGCAAGGACAACGCCAAGAACACACTCTATCTG | |
| | CAGATGAACAGCCTCAAGCCAGAGGACACTGGCATGTACTACTGCGCC | |
| | GCCGAGTTCGCCGATTGCAGCAGCAACTACTTTCTGCCTCCCGGCGCC | |
| | GTCAGATATTGGGGCCAAGGCACTCAAGTGACAGTCTCGAGCGGCGGA | |
| | GGATCCCAAGTGCAGCTGCAAGAGAGCGGAGGAGGAAGCGTGCAAGCC | |
| | GGAGGCTCTCTGAGACTGAGCTGTGCCGCCTCTAGGTATCTGTACAGC | |
| | ATCGACTACATGGCTTGGTTCAGACAGAGCCCCGGCAAGGAGAGGGAG | |
| | CCAGTGGCTGTCATCTACACTGCCTCCGGCGCCACATTCTATCCAGAT | |
| | AGCGTGAAGGGAAGGTTCACTATCAGCCAAGATAACGCCAAGATGACA | |
| | GTGTATCTGCAGATGAACTCTCTGAAGAGCGAGGACACTGCCATGTAC | |
| | TACTGTGCCGCCGTGAGGAAGACAGATAGCTACCTCTTCGACGCCCAG | |
| | AGCTTCACATACTGGGGCCAAGGCACACAAGTGACAGTCTCGTCTGCT | |
| | AGCCACCATCACCATCACCAC | |
| 334 | CAAGTGCAGCTGCAAGAGAGCGGAGGAGGCAGCGTCGAAGCTGGAGGA | 432 |
| | TCTCTGAGGCTGAGCTGTGCTGCCAGCGGCTACACTCACAGCAGCTAC | |
| | TGTATGGGCTGGTTCAGACAAGCCCCCGGCAAGGAGAGGGAAGGCGTG | |
| | GCTGCCATCGACGTGGATGGCAGCACTACTTACGCCGACAGCGTGAAG | |
| | GGAAGGTTCACTATCAGCAAGGACAACGCCAAGAACACACTCTATCTG | |
| | CAGATGAACAGCCTCAAGCCAGAGGACACTGGCATGTACTACTGCGCC | |
| | GCCGAGTTCGCCGATTGCAGCAGCAACTACTTTCTGCCTCCCGGCGCC | |
| | GTCAGATATTGGGGCCAAGGCACTCAAGTGACAGTCTCGAGCGGCGGA | |
| | GGATCCCAAGTGCAGCTGCAAGAGAGCGGAGGAGGAAGCGTGCAAGCC | |
| | GGAGGCTCTCTGAGGCTGAGCTGTGCCGCCTCTAGGTTCACATACAGC | |
| | AGCTACTGCATGGGCTGGTTCAGACAAGCCCCCGGCAAAGAGAGAGAA | |
| | GGCGTGGCCAGCATCGATAGCGATGGCTCCACTAGCTACACTGACAGC | |
| | GTGAAGGGAAGGTTCACTATCAGCAAGGACAACGCCAAGAACACTCTG | |
| | TATCTGCAGATGAACTCTCTGAAGCCAGAGGACACAGCCATGTACTAC | |
| | TGTGCCCTCGATCTGATGAGCACAGTGGTGCCCGGCTTCTGTGGCTTT | |
| | CTGCTGAGCGCTGGCATGGATTACTGGGGCAAGGGCACTCAAGTGACT | |
| | GTCTCGTCTGCTAGCCACCATCACCATCACCAC | |
| 335 | CAAGTGCAGCTGCAAGAGAGCGGAGGAGGCAGCGTCGAAGCTGGAGGA | 433 |
| | TCTCTGAGGCTGAGCTGTGCTGCCAGCGGCTACACTCACAGCAGCTAC | |
| | TGTATGGGCTGGTTCAGACAAGCCCCCGGCAAGGAGAGGGAAGGCGTG | |
| | GCTGCCATCGACGTGGATGGCAGCACTACTTACGCCGACAGCGTGAAG | |
| | GGAAGGTTCACTATCAGCAAGGACAACGCCAAGAACACACTCTATCTG | |
| | CAGATGAACAGCCTCAAGCCAGAGGACACTGGCATGTACTACTGCGCC | |
| | GCCGAGTTCGCCGATTGCAGCAGCAACTACTTTCTGCCTCCCGGCGCC | |
| | GTCAGATATTGGGGCCAAGGCACTCAAGTGACAGTCTCGAGCGGCGGA | |
| | GGATCCCAAGTGCAGCTGCAAGAGTCCGGAGGAGGCAGCGTCCAAGCC | |
| | GGAGGCTCTCTGAGGCTGAGCTGTGCTGCCAGCGGCTACACTTACAGC | |
| | ATGTACTGCATGGGCTGGTTCAGACAAGCCCCCGGCAAGGAAAGAGAG | |
| | GGCGTGGCCCAGATCAATAGCGATGGCAGCACAAGCTACGCCGACAGC | |
| | GTGAAGGGAAGGTTCACTATCTCCAAGGACAACGCCAAGAACACTCTG | |
| | TATCTGCAGATGAACTCTCTGAAGCCAGAGGACACTGCCATGTACTAC | |
| | TGCGCTGCCGATTCTAGGGTGTACGGCGGCAGCTGGTATGAGAGGCTC | |
| | TGCGGCCCTTACACATACGAGTACAACTACTGGGGCCAAGGCACACAA | |
| | GTGACTGTCTCGTCTGCTAGCCACCATCACCATCACCAC | |
| 336 | CAAGTGCAGCTGCAAGAGAGCGGAGGAGGCAGCGTCGAAGCTGGAGGA | 434 |
| | TCTCTGAGGCTGAGCTGTGCTGCCAGCGGCTACACTCACAGCAGCTAC | |
| | TGTATGGGCTGGTTCAGACAAGCCCCCGGCAAGGAGAGGGAAGGCGTG | |
| | GCTGCCATCGACGTGGATGGCAGCACTACTTACGCCGACAGCGTGAAG | |
| | GGAAGGTTCACTATCAGCAAGGACAACGCCAAGAACACACTCTATCTG | |
| | CAGATGAACAGCCTCAAGCCAGAGGACACTGGCATGTACTACTGCGCC | |
| | GCCGAGTTCGCCGATTGCAGCAGCAACTACTTTCTGCCTCCCGGCGCC | |
| | GTCAGATATTGGGGCCAAGGCACTCAAGTGACAGTCTCGAGCGGCGGA | |
| | GGATCCCAAGTGCAGCTGCAAGAGAGCGGCGGAGGAAGCGTGCAAGCC | |
| | GGAGGATCTCTGAGACTGAGCTGCGCTGTGAGCGGCTACGCCTACTCC | |
| | ACATACTGCATGGGCTGGTTTAGGCAAGCCCCCGGCAAAGAGAGAGAG | |
| | GGCGTGGCTGCTATCGATAGCGGCGGCAGCACAAGCTACGCCGATAGC | |
| | GTGAAGGGAAGGTTCACAATCAGCAAGGACAACGCCAAGAACACACTG | |
| | TATCTGAGGATGAACTCTCTGAAGCCAGAGGACACAGCCATGTACTAC | |
| | TGTGCTGCTGTGCCTCCTCCTCCAGATGGCGGCAGCTGTCTGTTTCTG | |
| | GGACCAGAGATCAAGGTCAGCAAGGCCGATTTTAGGTACTGGGGCCAA | |
| | GGCACACAAGTGACAGTCTCGTCTGCTAGCCACCATCACCATCACCAC | |
| 337 | CAAGTGCAGCTGCAAGAGAGCGGAGGAGGCAGCGTCGAAGCTGGAGGA | 435 |
| | TCTCTGAGGCTGAGCTGTGCTGCCAGCGGCTACACTCACAGCAGCTAC | |
| | TGTATGGGCTGGTTCAGACAAGCCCCCGGCAAGGAGAGGGAAGGCGTG | |
| | GCTGCCATCGACGTGGATGGCAGCACTACTTACGCCGACAGCGTGAAG | |
| | GGAAGGTTCACTATCAGCAAGGACAACGCCAAGAACACACTCTATCTG | |
| | CAGATGAACAGCCTCAAGCCAGAGGACACTGGCATGTACTACTGCGCC | |
| | GCCGAGTTCGCCGATTGCAGCAGCAACTACTTTCTGCCTCCCGGCGCC | |
| | GTCAGATATTGGGGCCAAGGCACTCAAGTGACAGTCTCGAGCGGCGGA | |
| | GGATCCCAAGTGCAGCTGCAAGAGAGCGGAGGAGGAAGCGTGCAAGCC | |
| | GGAGGCTCTCTGAGGCTGAGCTGTACAGTGTCCGGCTACACTTACAGC | |
| | TCCAATTGCATGGGCTGGTTTAGGCAAGCCCCCGGCAAGGAAAGAGAG | |
| | GGCGTGGCCACTATCTACACTGGCGGCGGCAACACATACTACGCCGAT | |
| | AGCGTGAAGGGAAGGTTCACTATCAGCCAAGATAACGCCAAGAACACA | |
| | GTGTATCTGCAGATGAACAATCTGAAGCCAGAGGACACTGCCATGTAC | |
| | TACTGTGCTGCTGAGCCACTGTCTAGGGTGTACGGCGGCAGCTGCCCA | |
| | ACTCCTACATTCGACTACTGGGGCCAAGGCACACAAGTGACTGTCTCG | |
| | TCTGCTAGCCACCATCACCATCACCAC | |
| 338 | CAAGTGCAGCTGCAAGAGAGCGGAGGAGGCAGCGTCGAAGCTGGAGGA | 436 |
| | TCTCTGAGGCTGAGCTGTGCTGCCAGCGGCTACACTCACAGCAGCTAC | |
| | TGTATGGGCTGGTTCAGACAAGCCCCCGGCAAGGAGAGGGAAGGCGTG | |
| | GCTGCCATCGACGTGGATGGCAGCACTACTTACGCCGACAGCGTGAAG | |
| | GGAAGGTTCACTATCAGCAAGGACAACGCCAAGAACACACTCTATCTG | |
| | CAGATGAACAGCCTCAAGCCAGAGGACACTGGCATGTACTACTGCGCC | |
| | GCCGAGTTCGCCGATTGCAGCAGCAACTACTTTCTGCCTCCCGGCGCC | |
| | GTCAGATATTGGGGCCAAGGCACTCAAGTGACAGTCTCGAGCGGCGGA | |
| | GGATCCCAAGTGCAGCTGCAAGAGAGCGGAGGAGGAAGCGTCCAAGCC | |
| | GGAGGCTCTCTGAGGCTGAGCTGTGGAGCCAGCGGCTACACTTACAGC | |
| | AGCTACTGTATGGGCTGGTTTAGGCAAGTGCCCGGCAAGGAGAGAGAG | |
| | GGCGTGGCCGTGATCGATTCCGATGGCAGCACAAGCTACGCTGACAGC | |
| | GTGAAGGGAAGGTTCACAATCAGCAAGGACAACGGCAAGAACACACTC | |
| | TATCTGCAGATGAACAGCCTCAAGCCAGAGGACACAGCCATGTACTAC | |
| | TGCGCCGCTGATCTGGGCCACTATAGGCCTCCTTGTGGCGTGCTGTAT | |
| | CTGGGCATGGATTACTGGGGCAAGGGCACACAAGTGACAGTCTCGTCT | |
| | GCTAGCCACCATCACCATCACCAC | |
| 339 | CAAGTGCAGCTGCAAGAGAGCGGAGGAGGCAGCGTCGAAGCTGGAGGA | 437 |
| | TCTCTGAGGCTGAGCTGTGCTGCCAGCGGCTACACTCACAGCAGCTAC | |
| | TGTATGGGCTGGTTCAGACAAGCCCCCGGCAAGGAGAGGGAAGGCGTG | |
| | GCTGCCATCGACGTGGATGGCAGCACTACTTACGCCGACAGCGTGAAG | |
| | GGAAGGTTCACTATCAGCAAGGACAACGCCAAGAACACACTCTATCTG | |
| | CAGATGAACAGCCTCAAGCCAGAGGACACTGGCATGTACTACTGCGCC | |
| | GCCGAGTTCGCCGATTGCAGCAGCAACTACTTTCTGCCTCCCGGCGCC | |
| | GTCAGATATTGGGGCCAAGGCACTCAAGTGACAGTCTCGAGCGGCGGA | |
| | GGATCCCAAGTGCAGCTGCAAGAGAGCGGAGGAGGAAGCGTCCAAGCC | |
| | GGAGGCTCTCTGAGACTGAGCTGTGCCGCCAGCGGCTACTCCAACTGC | |
| | AGCTACGACATGACTTGGTATAGGCAAGCCCCCGGCAAGGAGAGGGAG | |
| | TTCGTGTCCGCCATCCACAGCGACGGCAGCACTAGATACGCCGACAGC | |
| | GTGAAGGGAAGGTTCTTCATCAGCCAAGATAACGCCAAGAACACAGTG | |
| | TATCTGCAGATGAACTCCCTCAAGCCAGAGGACACTGCCATGTACTAC | |
| | TGCAAGACAGACCCACTGCACTGCAGAGCCCATGGCGGCAGCTGGTAT | |
| | AGCGTGAGGGCCAACTACTGGGGCCAAGGCACACAAGTGACAGTCTCG | |
| | TCTGCTAGCCACCATCACCATCACCAC | |
| 340 | CAAGTGCAGCTGCAAGAGAGCGGAGGAGGAAGCGTCCAAGCCGGAGGA | 438 |
| | TCTCTGAGACTGAGCTGCGCCGCTAGTGGCTACTCCTACAGCAGCTAC | |
| | TGCATGGGCTGGTTTAGGCAAGCCCCCGGCAAGGAGAGAGAAGGCGTG | |
| | GCCACTATCGACAGCGACGGCATGACAAGGTACGCCGACAGCGTGAAG | |
| | GGAAGGTTCACAATCAGCAAGGACAACGCCAAGAACACACTGTATCTG | |
| | CAGATGAACTCTCTGAAGCCAGAGGACACTGCCATGTACTACTGTGCC | |
| | GCTCCTCTGTACGACTGTGATAGCGGCGCTGTGGGCAGAAATCCACCT | |
| | TATTGGGGCCAAGGCACTCAAGTGACAGTCTCGAGCGGCGGAGGATCC | |
| | CAAGTGCAGCTGCAAGAGAGCGGAGGAGGAAGCGTGCAAGCCGGAGGC | |
| | TCTCTGAGACTGAGCTGTGCCGCCTCTAGGTATCTGTACAGCATCGAC | |
| | TACATGGCTTGGTTCAGACAGAGCCCCGGCAAGGAGAGGGAGCCAGTG | |
| | GCTGTCATCTACACTGCCTCCGGCGCCACATTCTATCCAGATAGCGTG | |
| | AAGGGAAGGTTCACTATCAGCCAAGATAACGCCAAGATGACAGTGTAT | |
| | CTGCAGATGAACTCTCTGAAGAGCGAGGACACTGCCATGTACTACTGT | |
| | GCCGCCGTGAGGAAGACAGATAGCTACCTCTTCGACGCCCAGAGCTTC | |
| | ACATACTGGGGCCAAGGCACACAAGTGACAGTCTCGTCTGCTAGCCAC | |
| | CATCACCATCACCAC | |
| 341 | CAAGTGCAGCTGCAAGAGAGCGGAGGAGGAAGCGTCCAAGCCGGAGGA | 439 |
| | TCTCTGAGACTGAGCTGCGCCGCTAGTGGCTACTCCTACAGCAGCTAC | |
| | TGCATGGGCTGGTTTAGGCAAGCCCCCGGCAAGGAGAGAGAAGGCGTG | |
| | GCCACTATCGACAGCGACGGCATGACAAGGTACGCCGACAGCGTGAAG | |
| | GGAAGGTTCACAATCAGCAAGGACAACGCCAAGAACACACTGTATCTG | |
| | CAGATGAACTCTCTGAAGCCAGAGGACACTGCCATGTACTACTGTGCC | |
| | GCTCCTCTGTACGACTGTGATAGCGGCGCTGTGGGCAGAAATCCACCT | |
| | TATTGGGGCCAAGGCACTCAAGTGACAGTCTCGAGCGGCGGAGGATCC | |
| | CAAGTGCAGCTGCAAGAGAGCGGAGGAGGAAGCGTGCAAGCCGGAGGC | |
| | TCTCTGAGGCTGAGCTGTGCCGCCTCTAGGTTCACATACAGCAGCTAC | |
| | TGCATGGGCTGGTTCAGACAAGCCCCCGGCAAAGAGAGAGAAGGCGTG | |
| | GCCAGCATCGATAGCGATGGCTCCACTAGCTACACTGACAGCGTGAAG | |
| | GGAAGGTTCACTATCAGCAAGGACAACGCCAAGAACACTCTGTATCTG | |
| | CAGATGAACTCTCTGAAGCCAGAGGACACAGCCATGTACTACTGTGCC | |
| | CTCGATCTGATGAGCACAGTGGTGCCCGGCTTCTGTGGCTTTCTGCTG | |
| | AGCGCTGGCATGGATTACTGGGGCAAGGGCACTCAAGTGACTGTCTCG | |
| | TCTGCTAGCCACCATCACCATCACCAC | |
| 342 | CAAGTGCAGCTGCAAGAGAGCGGAGGAGGAAGCGTCCAAGCCGGAGGA | 440 |
| | TCTCTGAGACTGAGCTGCGCCGCTAGTGGCTACTCCTACAGCAGCTAC | |
| | TGCATGGGCTGGTTTAGGCAAGCCCCCGGCAAGGAGAGAGAAGGCGTG | |
| | GCCACTATCGACAGCGACGGCATGACAAGGTACGCCGACAGCGTGAAG | |
| | GGAAGGTTCACAATCAGCAAGGACAACGCCAAGAACACACTGTATCTG | |
| | CAGATGAACTCTCTGAAGCCAGAGGACACTGCCATGTACTACTGTGCC | |
| | GCTCCTCTGTACGACTGTGATAGCGGCGCTGTGGGCAGAAATCCACCT | |
| | TATTGGGGCCAAGGCACTCAAGTGACAGTCTCGAGCGGCGGAGGATCC | |
| | CAAGTGCAGCTGCAAGAGTCCGGAGGAGGCAGCGTCCAAGCCGGAGGC | |
| | TCTCTGAGGCTGAGCTGTGCTGCCAGCGGCTACACTTACAGCATGTAC | |
| | TGCATGGGCTGGTTCAGACAAGCCCCCGGCAAGGAAAGAGAGGGCGTG | |
| | GCCCAGATCAATAGCGATGGCAGCACAAGCTACGCCGACAGCGTGAAG | |
| | GGAAGGTTCACTATCTCCAAGGACAACGCCAAGAACACTCTGTATCTG | |
| | CAGATGAACTCTCTGAAGCCAGAGGACACTGCCATGTACTACTGCGCT | |
| | GCCGATTCTAGGGTGTACGGCGGCAGCTGGTATGAGAGGCTCTGCGGC | |
| | CCTTACACATACGAGTACAACTACTGGGGCCAAGGCACACAAGTGACT | |
| | GTCTCGTCTGCTAGCCACCATCACCATCACCAC | |
| 343 | CAAGTGCAGCTGCAAGAGAGCGGAGGAGGAAGCGTCCAAGCCGGAGGA | 441 |
| | TCTCTGAGACTGAGCTGCGCCGCTAGTGGCTACTCCTACAGCAGCTAC | |
| | TGCATGGGCTGGTTTAGGCAAGCCCCCGGCAAGGAGAGAGAAGGCGTG | |
| | GCCACTATCGACAGCGACGGCATGACAAGGTACGCCGACAGCGTGAAG | |
| | GGAAGGTTCACAATCAGCAAGGACAACGCCAAGAACACACTGTATCTG | |
| | CAGATGAACTCTCTGAAGCCAGAGGACACTGCCATGTACTACTGTGCC | |
| | GCTCCTCTGTACGACTGTGATAGCGGCGCTGTGGGCAGAAATCCACCT | |
| | TATTGGGGCCAAGGCACTCAAGTGACAGTCTCGAGCGGCGGAGGATCC | |
| | CAAGTGCAGCTGCAAGAGAGCGGCGGAGGAAGCGTGCAAGCCGGAGGA | |
| | TCTCTGAGACTGAGCTGCGCTGTGAGCGGCTACGCCTACTCCACATAC | |
| | TGCATGGGCTGGTTTAGGCAAGCCCCCGGCAAAGAGAGAGAGGGCGTG | |
| | GCTGCTATCGATAGCGGCGGCAGCACAAGCTACGCCGATAGCGTGAAG | |
| | GGAAGGTTCACAATCAGCAAGGACAACGCCAAGAACACACTGTATCTG | |
| | AGGATGAACTCTCTGAAGCCAGAGGACACAGCCATGTACTACTGTGCT | |
| | GCTGTGCCTCCTCCTCCAGATGGCGGCAGCTGTCTGTTTCTGGGACCA | |
| | GAGATCAAGGTCAGCAAGGCCGATTTTAGGTACTGGGGCCAAGGCACA | |
| | CAAGTGACAGTCTCGTCTGCTAGCCACCATCACCATCACCAC | |
| 344 | CAAGTGCAGCTGCAAGAGAGCGGAGGAGGAAGCGTCCAAGCCGGAGGA | 442 |
| | TCTCTGAGACTGAGCTGCGCCGCTAGTGGCTACTCCTACAGCAGCTAC | |
| | TGCATGGGCTGGTTTAGGCAAGCCCCCGGCAAGGAGAGAGAAGGCGTG | |
| | GCCACTATCGACAGCGACGGCATGACAAGGTACGCCGACAGCGTGAAG | |
| | GGAAGGTTCACAATCAGCAAGGACAACGCCAAGAACACACTGTATCTG | |
| | CAGATGAACTCTCTGAAGCCAGAGGACACTGCCATGTACTACTGTGCC | |
| | GCTCCTCTGTACGACTGTGATAGCGGCGCTGTGGGCAGAAATCCACCT | |
| | TATTGGGGCCAAGGCACTCAAGTGACAGTCTCGAGCGGCGGAGGATCC | |
| | CAAGTGCAGCTGCAAGAGAGCGGAGGAGGAAGCGTGCAAGCCGGAGGC | |
| | TCTCTGAGGCTGAGCTGTACAGTGTCCGGCTACACTTACAGCTCCAAT | |
| | TGCATGGGCTGGTTTAGGCAAGCCCCCGGCAAGGAAAGAGAGGGCGTG | |
| | GCCACTATCTACACTGGCGGCGGCAACACATACTACGCCGATAGCGTG | |
| | AAGGGAAGGTTCACTATCAGCCAAGATAACGCCAAGAACACAGTGTAT | |
| | CTGCAGATGAACAATCTGAAGCCAGAGGACACTGCCATGTACTACTGT | |
| | GCTGCTGAGCCACTGTCTAGGGTGTACGGCGGCAGCTGCCCAACTCCT | |
| | ACATTCGACTACTGGGGCCAAGGCACACAAGTGACTGTCTCGTCTGCT | |
| | AGCCACCATCACCATCACCAC | |
| 345 | CAAGTGCAGCTGCAAGAGAGCGGAGGAGGAAGCGTCCAAGCCGGAGGA | 443 |
| | TCTCTGAGACTGAGCTGCGCCGCTAGTGGCTACTCCTACAGCAGCTAC | |
| | TGCATGGGCTGGTTTAGGCAAGCCCCCGGCAAGGAGAGAGAAGGCGTG | |
| | GCCACTATCGACAGCGACGGCATGACAAGGTACGCCGACAGCGTGAAG | |
| | GGAAGGTTCACAATCAGCAAGGACAACGCCAAGAACACACTGTATCTG | |
| | CAGATGAACTCTCTGAAGCCAGAGGACACTGCCATGTACTACTGTGCC | |
| | GCTCCTCTGTACGACTGTGATAGCGGCGCTGTGGGCAGAAATCCACCT | |
| | TATTGGGGCCAAGGCACTCAAGTGACAGTCTCGAGCGGCGGAGGATCC | |
| | CAAGTGCAGCTGCAAGAGAGCGGAGGAGGAAGCGTCCAAGCCGGAGGC | |
| | TCTCTGAGGCTGAGCTGTGGAGCCAGCGGCTACACTTACAGCAGCTAC | |
| | TGTATGGGCTGGTTTAGGCAAGTGCCCGGCAAGGAGAGAGAGGGCGTG | |
| | GCCGTGATCGATTCCGATGGCAGCACAAGCTACGCTGACAGCGTGAAG | |
| | GGAAGGTTCACAATCAGCAAGGACAACGGCAAGAACACACTCTATCTG | |
| | CAGATGAACAGCCTCAAGCCAGAGGACACAGCCATGTACTACTGCGCC | |
| | GCTGATCTGGGCCACTATAGGCCTCCTTGTGGCGTGCTGTATCTGGGC | |
| | ATGGATTACTGGGGCAAGGGCACACAAGTGACAGTCTCGTCTGCTAGC | |
| | CACCATCACCATCACCAC | |
| 346 | CAAGTGCAGCTGCAAGAGAGCGGAGGAGGAAGCGTCCAAGCCGGAGGA | 444 |
| | TCTCTGAGACTGAGCTGCGCCGCTAGTGGCTACTCCTACAGCAGCTAC | |
| | TGCATGGGCTGGTTTAGGCAAGCCCCCGGCAAGGAGAGAGAAGGCGTG | |
| | GCCACTATCGACAGCGACGGCATGACAAGGTACGCCGACAGCGTGAAG | |
| | GGAAGGTTCACAATCAGCAAGGACAACGCCAAGAACACACTGTATCTG | |
| | CAGATGAACTCTCTGAAGCCAGAGGACACTGCCATGTACTACTGTGCC | |
| | GCTCCTCTGTACGACTGTGATAGCGGCGCTGTGGGCAGAAATCCACCT | |
| | TATTGGGGCCAAGGCACTCAAGTGACAGTCTCGAGCGGCGGAGGATCC | |
| | CAAGTGCAGCTGCAAGAGAGCGGAGGAGGAAGCGTCCAAGCCGGAGGC | |
| | TCTCTGAGACTGAGCTGTGCCGCCAGCGGCTACTCCAACTGCAGCTAC | |
| | GACATGACTTGGTATAGGCAAGCCCCCGGCAAGGAGAGGGAGTTCGTG | |
| | TCCGCCATCCACAGCGACGGCAGCACTAGATACGCCGACAGCGTGAAG | |
| | GGAAGGTTCTTCATCAGCCAAGATAACGCCAAGAACACAGTGTATCTG | |
| | CAGATGAACTCCCTCAAGCCAGAGGACACTGCCATGTACTACTGCAAG | |
| | ACAGACCCACTGCACTGCAGAGCCCATGGCGGCAGCTGGTATAGCGTG | |
| | AGGGCCAACTACTGGGGCCAAGGCACACAAGTGACAGTCTCGTCTGCT | |
| | AGCCACCATCACCATCACCAC | |
| 347 | CAAGTGCAGCTGCAAGAGAGCGGAGGCGGCAGCGTGCAGACTGGAGGC | 445 |
| | TCTCTGAGACTGAGCTGTGCTGCCAGCGGCTACACTTATCTGAGGGGC | |
| | TGTATGGGCTGGTTTAGGCAAGCCCCCGGCAAGGAGAGAGAGGGCGTG | |
| | GCCGTCATGGATGTGGTGGGCGATAGGAGAAGCTACATCGACAGCGTG | |
| | AAGGGAAGGTTCACAATCTCTAGGGACAATGCCGCCAACAGCGTCTAT | |
| | CTGCAGATGGACAATCTGAAGCCAGAGGACACAGCCATGTACTACTGC | |
| | ACTGCCGGCCCTAACTGTGTGGGCTGGAGAAGCGGACTGGATTACTGG | |
| | GGCCAAGGCACACAAGTGACAGTCTCGAGCGGCGGAGGATCCCAAGTG | |
| | CAGCTGCAAGAGAGCGGAGGAGGAAGCGTGCAAGCCGGAGGCTCTCTG | |
| | AGACTGAGCTGTGCCGCCTCTAGGTATCTGTACAGCATCGACTACATG | |
| | GCTTGGTTCAGACAGAGCCCCGGCAAGGAGAGGGAGCCAGTGGCTGTC | |
| | ATCTACACTGCCTCCGGCGCCACATTCTATCCAGATAGCGTGAAGGGA | |
| | AGGTTCACTATCAGCCAAGATAACGCCAAGATGACAGTGTATCTGCAG | |
| | ATGAACTCTCTGAAGAGCGAGGACACTGCCATGTACTACTGTGCCGCC | |
| | GTGAGGAAGACAGATAGCTACCTCTTCGACGCCCAGAGCTTCACATAC | |
| | TGGGGCCAAGGCACACAAGTGACAGTCTCGTCTGCTAGCCACCATCAC | |
| | CATCACCAC | |
| 348 | CAAGTGCAGCTGCAAGAGAGCGGAGGCGGCAGCGTGCAGACTGGAGGC | 446 |
| | TCTCTGAGACTGAGCTGTGCTGCCAGCGGCTACACTTATCTGAGGGGC | |
| | TGTATGGGCTGGTTTAGGCAAGCCCCCGGCAAGGAGAGAGAGGGCGTG | |
| | GCCGTCATGGATGTGGTGGGCGATAGGAGAAGCTACATCGACAGCGTG | |
| | AAGGGAAGGTTCACAATCTCTAGGGACAATGCCGCCAACAGCGTCTAT | |
| | CTGCAGATGGACAATCTGAAGCCAGAGGACACAGCCATGTACTACTGC | |
| | ACTGCCGGCCCTAACTGTGTGGGCTGGAGAAGCGGACTGGATTACTGG | |
| | GGCCAAGGCACACAAGTGACAGTCTCGAGCGGCGGAGGATCCCAAGTG | |
| | CAGCTGCAAGAGAGCGGAGGAGGAAGCGTGCAAGCCGGAGGCTCTCTG | |
| | AGGCTGAGCTGTGCCGCCTCTAGGTTCACATACAGCAGCTACTGCATG | |
| | GGCTGGTTCAGACAAGCCCCCGGCAAAGAGAGAGAAGGCGTGGCCAGC | |
| | ATCGATAGCGATGGCTCCACTAGCTACACTGACAGCGTGAAGGGAAGG | |
| | TTCACTATCAGCAAGGACAACGCCAAGAACACTCTGTATCTGCAGATG | |
| | AACTCTCTGAAGCCAGAGGACACAGCCATGTACTACTGTGCCCTCGAT | |
| | CTGATGAGCACAGTGGTGCCCGGCTTCTGTGGCTTTCTGCTGAGCGCT | |
| | GGCATGGATTACTGGGGCAAGGGCACTCAAGTGACTGTCTCGTCTGCT | |
| | AGCCACCATCACCATCACCAC | |
| 349 | CAAGTGCAGCTGCAAGAGAGCGGAGGCGGCAGCGTGCAGACTGGAGGC | 447 |
| | TCTCTGAGACTGAGCTGTGCTGCCAGCGGCTACACTTATCTGAGGGGC | |
| | TGTATGGGCTGGTTTAGGCAAGCCCCCGGCAAGGAGAGAGAGGGCGTG | |
| | GCCGTCATGGATGTGGTGGGCGATAGGAGAAGCTACATCGACAGCGTG | |
| | AAGGGAAGGTTCACAATCTCTAGGGACAATGCCGCCAACAGCGTCTAT | |
| | CTGCAGATGGACAATCTGAAGCCAGAGGACACAGCCATGTACTACTGC | |
| | ACTGCCGGCCCTAACTGTGTGGGCTGGAGAAGCGGACTGGATTACTGG | |
| | GGCCAAGGCACACAAGTGACAGTCTCGAGCGGCGGAGGATCCCAAGTG | |
| | CAGCTGCAAGAGTCCGGAGGAGGCAGCGTCCAAGCCGGAGGCTCTCTG | |
| | AGGCTGAGCTGTGCTGCCAGCGGCTACACTTACAGCATGTACTGCATG | |
| | GGCTGGTTCAGACAAGCCCCCGGCAAGGAAAGAGAGGGCGTGGCCCAG | |
| | ATCAATAGCGATGGCAGCACAAGCTACGCCGACAGCGTGAAGGGAAGG | |
| | TTCACTATCTCCAAGGACAACGCCAAGAACACTCTGTATCTGCAGATG | |
| | AACTCTCTGAAGCCAGAGGACACTGCCATGTACTACTGCGCTGCCGAT | |
| | TCTAGGGTGTACGGCGGCAGCTGGTATGAGAGGCTCTGCGGCCCTTAC | |
| | ACATACGAGTACAACTACTGGGGCCAAGGCACACAAGTGACTGTCTCG | |
| | TCTGCTAGCCACCATCACCATCACCAC | |
| 350 | CAAGTGCAGCTGCAAGAGAGCGGAGGCGGCAGCGTGCAGACTGGAGGC | 448 |
| | TCTCTGAGACTGAGCTGTGCTGCCAGCGGCTACACTTATCTGAGGGGC | |
| | TGTATGGGCTGGTTTAGGCAAGCCCCCGGCAAGGAGAGAGAGGGCGTG | |
| | GCCGTCATGGATGTGGTGGGCGATAGGAGAAGCTACATCGACAGCGTG | |
| | AAGGGAAGGTTCACAATCTCTAGGGACAATGCCGCCAACAGCGTCTAT | |
| | CTGCAGATGGACAATCTGAAGCCAGAGGACACAGCCATGTACTACTGC | |
| | ACTGCCGGCCCTAACTGTGTGGGCTGGAGAAGCGGACTGGATTACTGG | |
| | GGCCAAGGCACACAAGTGACAGTCTCGAGCGGCGGAGGATCCCAAGTG | |
| | CAGCTGCAAGAGAGCGGCGGAGGAAGCGTGCAAGCCGGAGGATCTCTG | |
| | AGACTGAGCTGCGCTGTGAGCGGCTACGCCTACTCCACATACTGCATG | |
| | GGCTGGTTTAGGCAAGCCCCCGGCAAAGAGAGAGAGGGCGTGGCTGCT | |
| | ATCGATAGCGGCGGCAGCACAAGCTACGCCGATAGCGTGAAGGGAAGG | |
| | TTCACAATCAGCAAGGACAACGCCAAGAACACACTGTATCTGAGGATG | |
| | AACTCTCTGAAGCCAGAGGACACAGCCATGTACTACTGTGCTGCTGTG | |
| | CCTCCTCCTCCAGATGGCGGCAGCTGTCTGTTTCTGGGACCAGAGATC | |
| | AAGGTCAGCAAGGCCGATTTTAGGTACTGGGGCCAAGGCACACAAGTG | |
| | ACAGTCTCGTCTGCTAGCCACCATCACCATCACCAC | |
| 351 | CAAGTGCAGCTGCAAGAGAGCGGAGGCGGCAGCGTGCAGACTGGAGGC | 449 |
| | TCTCTGAGACTGAGCTGTGCTGCCAGCGGCTACACTTATCTGAGGGGC | |
| | TGTATGGGCTGGTTTAGGCAAGCCCCCGGCAAGGAGAGAGAGGGCGTG | |
| | GCCGTCATGGATGTGGTGGGCGATAGGAGAAGCTACATCGACAGCGTG | |
| | AAGGGAAGGTTCACAATCTCTAGGGACAATGCCGCCAACAGCGTCTAT | |
| | CTGCAGATGGACAATCTGAAGCCAGAGGACACAGCCATGTACTACTGC | |
| | ACTGCCGGCCCTAACTGTGTGGGCTGGAGAAGCGGACTGGATTACTGG | |
| | GGCCAAGGCACACAAGTGACAGTCTCGAGCGGCGGAGGATCCCAAGTG | |
| | CAGCTGCAAGAGAGCGGAGGAGGAAGCGTGCAAGCCGGAGGCTCTCTG | |
| | AGGCTGAGCTGTACAGTGTCCGGCTACACTTACAGCTCCAATTGCATG | |
| | GGCTGGTTTAGGCAAGCCCCCGGCAAGGAAAGAGAGGGCGTGGCCACT | |
| | ATCTACACTGGCGGCGGCAACACATACTACGCCGATAGCGTGAAGGGA | |
| | AGGTTCACTATCAGCCAAGATAACGCCAAGAACACAGTGTATCTGCAG | |
| | ATGAACAATCTGAAGCCAGAGGACACTGCCATGTACTACTGTGCTGCT | |
| | GAGCCACTGTCTAGGGTGTACGGCGGCAGCTGCCCAACTCCTACATTC | |
| | GACTACTGGGGCCAAGGCACACAAGTGACTGTCTCGTCTGCTAGCCAC | |
| | CATCACCATCACCAC | |
| 352 | CAAGTGCAGCTGCAAGAGAGCGGAGGCGGCAGCGTGCAGACTGGAGGC | 450 |
| | TCTCTGAGACTGAGCTGTGCTGCCAGCGGCTACACTTATCTGAGGGGC | |
| | TGTATGGGCTGGTTTAGGCAAGCCCCCGGCAAGGAGAGAGAGGGCGTG | |
| | GCCGTCATGGATGTGGTGGGCGATAGGAGAAGCTACATCGACAGCGTG | |
| | AAGGGAAGGTTCACAATCTCTAGGGACAATGCCGCCAACAGCGTCTAT | |
| | CTGCAGATGGACAATCTGAAGCCAGAGGACACAGCCATGTACTACTGC | |
| | ACTGCCGGCCCTAACTGTGTGGGCTGGAGAAGCGGACTGGATTACTGG | |
| | GGCCAAGGCACACAAGTGACAGTCTCGAGCGGCGGAGGATCCCAAGTG | |
| | CAGCTGCAAGAGAGCGGAGGAGGAAGCGTCCAAGCCGGAGGCTCTCTG | |
| | AGGCTGAGCTGTGGAGCCAGCGGCTACACTTACAGCAGCTACTGTATG | |
| | GGCTGGTTTAGGCAAGTGCCCGGCAAGGAGAGAGAGGGCGTGGCCGTG | |
| | ATCGATTCCGATGGCAGCACAAGCTACGCTGACAGCGTGAAGGGAAGG | |
| | TTCACAATCAGCAAGGACAACGGCAAGAACACACTCTATCTGCAGATG | |
| | AACAGCCTCAAGCCAGAGGACACAGCCATGTACTACTGCGCCGCTGAT | |
| | CTGGGCCACTATAGGCCTCCTTGTGGCGTGCTGTATCTGGGCATGGAT | |
| | TACTGGGGCAAGGGCACACAAGTGACAGTCTCGTCTGCTAGCCACCAT | |
| | CACCATCACCAC | |
| 353 | CAAGTGCAGCTGCAAGAGAGCGGAGGCGGCAGCGTGCAGACTGGAGGC | 451 |
| | TCTCTGAGACTGAGCTGTGCTGCCAGCGGCTACACTTATCTGAGGGGC | |
| | TGTATGGGCTGGTTTAGGCAAGCCCCCGGCAAGGAGAGAGAGGGCGTG | |
| | GCCGTCATGGATGTGGTGGGCGATAGGAGAAGCTACATCGACAGCGTG | |
| | AAGGGAAGGTTCACAATCTCTAGGGACAATGCCGCCAACAGCGTCTAT | |
| | CTGCAGATGGACAATCTGAAGCCAGAGGACACAGCCATGTACTACTGC | |
| | ACTGCCGGCCCTAACTGTGTGGGCTGGAGAAGCGGACTGGATTACTGG | |
| | GGCCAAGGCACACAAGTGACAGTCTCGAGCGGCGGAGGATCCCAAGTG | |
| | CAGCTGCAAGAGAGCGGAGGAGGAAGCGTCCAAGCCGGAGGCTCTCTG | |
| | AGACTGAGCTGTGCCGCCAGCGGCTACTCCAACTGCAGCTACGACATG | |
| | ACTTGGTATAGGCAAGCCCCCGGCAAGGAGAGGGAGTTCGTGTCCGCC | |
| | ATCCACAGCGACGGCAGCACTAGATACGCCGACAGCGTGAAGGGAAGG | |
| | TTCTTCATCAGCCAAGATAACGCCAAGAACACAGTGTATCTGCAGATG | |
| | AACTCCCTCAAGCCAGAGGACACTGCCATGTACTACTGCAAGACAGAC | |
| | CCACTGCACTGCAGAGCCCATGGCGGCAGCTGGTATAGCGTGAGGGCC | |
| | AACTACTGGGGCCAAGGCACACAAGTGACAGTCTCGTCTGCTAGCCAC | |
| | CATCACCATCACCAC | |

TABLE 15
IL10Ra and IL10Rb Receptor Subunits and IL10 Amino
Acid Sequences

| | | SEQ |
|---|---|---|
| Description | Amino Acid Sequence | ID NQ |
| IL10RA | MLPCLVVLLAALLSLRLGSDAHGTELPSPPSVWFEAEFFHHILH | 452 |
| Human | WTPIPNQSESTCYEVALLRYGIESWNSISNCSQTLSYDLTAVTL | |
| PreProtein | DLYHSNGYRARVRAVDGSRHSNWTVTNTRFSVDEVTLTVGSVNL | |
| | EIHNGFILGKIQLPRPKMAPANDTYESIFSHFREYEIAIRKVPG | |
| | NFTFTHKKVKHENFSLLTSGEVGEFCVQVKPSVASRSNKGMWSK | |
| | EECISLTRQYFTVTNVIIFFAFVLLLSGALAYCLALQLYVRRRK | |
| | KLPSVLLFKKPSPFIFISQRPSPETQDTIHPLDEEAFLKVSPEL | |
| | KNLDLHGSTDSGFGSTKPSLQTEEPQFLLPDPHPQADRTLGNRE | |
| | PPVLGDSCSSGSSNSTDSGICLQEPSLSPSTGPTWEQQVGSNSR | |
| | GQDDSGIDLVQNSEGRAGDTQGGSALGHHSPPEPEVPGEEDPAA | |
| | VAFQGYLRQTRCAEEKATKTGCLEEESPLTDGLGPKFGRCLVDE | |
| | AGLHPPALAKGYLKQDPLEMTLASSGAPTGQWNQPTEEWSLLAL | |
| | SSCSDLGISDWSFAHDLAPLGCVAAPGGLLGSFNSDLVTLPLIS | |
| | SLQSSE | |
| IL10RA | HGTELPSPPSVWFEAEFFHHILHWTPIPNQSESTCYEVALLRYG | 453 |
| Human | IESWNSISNCSQTLSYDLTAVTLDLYHSNGYRARVRAVDGSRHS | |
| Extracellular | NWTVTNTRFSVDEVTLTVGSVNLEIHNGFILGKIQLPRPKMAPA | |
| Domain | NDTYESIFSHFREYEIAIRKVPGNFTFTHKKVKHENFSLLTSGE | |
| | VGEFCVQVKPSVASRSNKGMWSKEECISLTRQYFTVTN | |
| IL10RA | MLSRLLPFLVTISSLSLEFIAYGTELPSPSYVWFEARFFQHILH | 454 |
| Mouse | WKPIPNQSESTYYEVALKQYGNSTWNDIHICRKAQALSCDLTTF | |
| Preprotein | TLDLYHRSYGYRARVRAVDNSQYSNWTTTETRFTVDEVILTVDS | |
| | VTLKAMDGIIYGTIHPPRPTITPAGDEYEQVFKDLRVYKISIRK | |
| | FSELKNATKRVKQETFTLTVPIGVRKFCVKVLPRLESRINKAEW | |
| | SEEQCLLITTEQYFTVTNLSILVISMLLFCGILVCLVLQWYIRH | |
| | PGKLPTVLVFKKPHDFFPANPLCPETPDAIHIVDLEVFPKVSLE | |
| | LRDSVLHGSTDSGFGSGKPSLQTEESQFLLPGSHPQIQGTLGKE | |
| | ESPGLQATCGDNTDSGICLQEPGLHSSMGPAWKQQLGYTHQDQD | |
| | DSDVNLVQNSPGQPKYTQDASALGHVCLLEPKAPEEKDQVMVTF | |
| | QGYQKQTRWKAEAAGPAECLDEEIPLTDAFDPELGVHLQDDLAW | |
| | PPPALAAGYLKQESQGMASAPPGTPSRQWNQLTEEWSLLGVVSC | |
| | EDLSIESWRFAHKLDPLDCGAAPGGLLDSLGSNLVTLPLISSLQ | |
| | VEE | |
| IL10RA | LEFIAYGTELPSPSYVWFEARFFQHILHWKPIPNQSESTYYEVA | 455 |
| ECD | LKQYGNSTWNDIHICRKAQALSCDLTTFTLDLYHRSYGYRARVR | |
| | AVDNSQYSNWTTTETRFTVDEVILTVDSVTLKAMDGIIYGTIHP | |
| | PRPTITPAGDEYEQVFKDLRVYKISIRKFSELKNATKRVKQETF | |
| | TLTVPIGVRKFCVKVLPRLESRINKAEWSEEQCLLITTEQYFTV | |
| | TNLSI | |
| IL10Rb | MAWSLGSWLGGCLLVSALGMVPPPENVRMNSVNFKNILQWESPA | 456 |
| Human | FAKGNLTFTAQYLSYRIFQDKCMNTTLTECDFSSLSKYGDHTLR | |
| Pre- | VRAEFADEHSDWVNITFCPVDDTIIGPPGMQVEVLADSLHMRFL | |
| protein | APKIENEYETWTMKNVYNSWTYNVQYWKNGTDEKFQITPQYDFE | |
| | VLRNLEPWTTYCVQVRGFLPDRNKAGEWSEPVCEQTTHDETVPS | |
| | WMVAVILMASVFMVCLALLGCFALLWCVYKKTKYAFSPRNSLPQ | |
| | HLKEFLGHPHHNTLLFFSFPLSDENDVFDKLSVIAEDSESGKQN | |
| | PGDSCSLGTPPGQGPQS | |
| IL10Rb | MVPPPENVRMNSVNFKNILQWESPAFAKGNLTFTAQYLSYRIFQ | 457 |
| Human ECD | DKCMNTTLTECDFSSLSKYGDHTLRVRAEFADEHSDWVNITFCP | |
| | VDDTIIGPPGMQVEVLADSLHMRFLAPKIENEYETWTMKNVYNS | |
| | WTYNVQYWKNGTDEKFQITPQYDFEVLRNLEPWTTYCVQVRGFL | |
| | PDRNKAGEWSEPVCEQTTHDETVPS | |
| IL10Rb | MAPCVAGWLGGFLLVPALGIPPPEKVRMNSVNFKNILQWEVPAF | 458 |
| Murine | PKTNLTFTAQYESYRSFQDHCKRTASTQCDFSHLSKYGDYTVRV | |
| Pre- | RAELADEHSEWVNVTFCPVEDTIIGPPEMQIESLAESLHLRFSA | |
| Protein | PQIENEPETWTLKNIYDSWAYRVQYWKNGTNEKFQVVSPYDSEV | |
| | LRNLEPWTTYCIQVQGFLLDQNRTGEWSEPICERTGNDEITPSW | |
| | IVAIILIVSVLVVFLFLLGCFVVLWLIYKKTKHTFRSGTSLPQH | |
| | LKEFLGHPHHSTFLLFSFPPPEEAEVFDKLSIISEESEGSKQSP | |
| | EDNCASEPPSDPGPRELESKDEAPSPPHDDPKLLTSTSEV | |
| IL10Rb | MIPPPEKVRMNSVNFKNILQWEVPAFPKTNLTFTAQYESYRSFQ | 459 |
| Murine | DHCKRTASTQCDFSHLSKYGDYTVRVRAELADEHSEWVNVTFCP | |
| ECD | VEDTIIGPPEMQIESLAESLHLRFSAPQIENEPETWTLKNIYDS | |
| | WAYRVQYWKNGTNEKFQVVSPYDSEVLRNLEPWTTYCIQVQGFL | |
| | LDQNRTGEWSEPICERTGNDEITPS | |
| IL10 | SPGQGTQSENSCTHFPGNLPNMLRDLRDAFSRVKTFFQMKDQLD | 460 |
| Human | NLLLKESLLEDFKGYLGCQALSEMIQFYLEEVMPQAENQDPDIK | |
| Mature | AHVNSLGENLKTLRLRLRRCHRFLPCENKSKAVEQVKNAFNKLQ | |
| Protein | EKGIYKAMSEFDIFINYIEAYMTMKIRN | |
| IL10 | SRGQYSREDNNCTHFPVGQSHMLLELRTAFSQVKTFFQTKDQLD | 461 |
| Mouse | NILLTDSLMQDFKGYLGCQALSEMIQFYLVEVMPQAEKHGPEIK | |
| Mature | EHLNSLGEKLKTLRMRLRRCHRFLPCENKSKAVEQVKSDFNKLQ | |
| Protein | DQGVYKAMNEFDIFINCIEAYMMIKMKS | |

TABLE 16
Exemplary Linkers

| | | SEQ |
|---|---|---|
| Abbreviation | Amino Acid Sequence | ID NO |
| G4S | GGGGS | 462 |
| G5S | GGGGGS | 463 |
| GGSG | GGSG | 464 |
| SG3 | SGGG | 465 |
| GS² | GSGS | 466 |
| GS³ | GSGSGS | 467 |
| GS⁴ | GSGSGSGSGS | 468 |
| GS⁵ | GSGSGSGSGSGS | 469 |
| 6X, GGS² | GGSGGS | 470 |
| GGS3 | GGSGGSGGS | 471 |
| 10x | GGSGGSGGSG | 472 |
| GGSG² | GGSGGGSG | 473 |
| GGSG³ | GGSGGGSGGGSG | 474 |
| GGSG⁴ | GGSGGGSGGGSGGGSG | 475 |
| GGSG⁵ | GGSGGGSGGGSGGGSGGGSG | 476 |
| G4S² | GGGGSGGGGS | 477 |
| 8X, GGGS | GGGSGGGS | 478 |
| GGSGG | GGSGG | 479 |
| GSGSG | GSGSG | 480 |
| GSGG | GSGGG | 481 |
| GGGSG | GGGSG | 482 |
| GSSSG | GSSSG | 483 |
| 4X, G3S | GGGS | 484 |
| IgG4 Hinge | ASRVESKYGPPCPPCPAPEFLGGPSVFLFPPKPKDTLMIS | 485 |
| | RTPEVTCVVVDVSQEDPEVQFNWYVDGVEVHNAKTKPREE | |
| | QFGSTYRVVSVLTVLHQDWLNGKEYKCKVSNKGLPSSIEK | |
| | TISKAKGQPREPQVYTLPPSQEEMTKNQVSLTCLVKGFYP | |
| | SDIAVEWESNGQPENNYKTTPPVLDSDGSFFLYSRLTVDK | |
| | SRWQEGNVFSCSVMHEALHNHYTQKSLSLSLG | |

TABLE 17
Exemplary IL10Ra/Rb Binding Molecules

| Name | Amino Acid Sequence | SEQ ID NO |
|---|---|---|
| DR838 | EVQLQESGGGSVQAGGSLRLSCAASGYSQCSYDMTWYRQAPGKER | 486 |
| | EFVSAIHSDGSTRYADSVKGRFFISQDNAKNTVYLQMNSLKPEDT | |
| | AMYYCKTDPLHCRAHGGSWYSVRANYWGQGTQVTVSGGGSQVQLQ | |
| | ESGGGSVEAGGSLRLSCAASGYTHSSYCMGWFRQAPGKEREGVAA | |
| | IDVDGSTTYADSVKGRFTISKDNAKNTLYLQMNSLKPEDTGMYYC | |
| | AAEFADCSSNYFLPPGAVRYWGQGTQVTVSSASHHHHHH | |
| DR839 | EVQLQESGGGSVQAGGSLRLSCAASGYSDCSYDMTWYRQAPGKER | 487 |
| | EFVSAIHSDGSTRYADSVKGRFFISQDNAKNTVYLQMNSLKPEDT | |
| | AMYYCKTDPLHCRAHGGSWYSVRANYWGQGTQVTVSSGGGSQVQL | |
| | QESGGGSVEAGGSLRLSCAASGYTHSSYCMGWFRQAPGKEREGVA | |
| | AIDVDGSTTYADSVKGRFTISKDNAKNTLYLQMNSLKPEDTGMYY | |
| | CAAEFADCSSNYFLPPGAVRYWGQGTQVTVSSASHHHHHH | |
| DR841 | EVQLQESGGGSVQAGGSLRLSCAASGYSNCAYDMTWYRQAPGKER | 488 |
| | EFVSAIHSDGSTRYADSVKGRFFISQDNAKNTVYLQMNSLKPEDT | |
| | AMYYCKTDPLHCRAHGGSWYSVRANYWGQGTQVTVSSGGGSQVQL | |
| | QESGGGSVEAGGSLRLSCAASGYTHSSYCMGWFRQAPGKEREGVA | |
| | AIDVDGSTTYADSVKGRFTISKDNAKNTLYLQMNSLKPEDTGMYY | |
| | CAAEFADCSSNYFLPPGAVRYWGQGTQVTVSSASHHHHHH | |
| DR890 | EVQLQESGGGSVQAGGSLRLSCAASGYSNCSYDMTWYRQAPGKER | 489 |
| | EFVSAIHSDGSTRYADSVKGRFFISQDNAKNTVYLQMNSLKPEDT | |
| | AMYYCKTDPLHCRAHGGSWYSVRANYWGQGTQVTVSSQVQLQESG | |
| | GGSVQAGGSLRLSCAASRYTYNSYCMGWFRQAPGKEREGVATIDS | |
| | DGMTRYADSVKGRFTISKDNAKNTLYLQMNSLKPEDTAMYYCAAD | |
| | ADCTIAAMTTNPLGQGTQVTVSSASHHHHHH | |
| DR891 | EVQLQESGGGSVQAGGSLRLSCAASGYSNCSYDMTWYRQAPGKER | 490 |
| | EFVSAIHSDGSTRYADSVKGRFFISQDNAKNTVYLQMNSLKPEDT | |
| | AMYYCKTDPLHCRAHGGSWYSVRANYWGQGTQVTVSSGSQVQLQE | |
| | SGGGSVQAGGSLRLSCAASRYTYNSYCMGWFRQAPGKEREGVATI | |
| | DSDGMTRYADSVKGRFTISKDNAKNTLYLQMNSLKPEDTAMYYCA | |
| | ADADCTIAAMTTNPLGQGTQVTVSSASHHHHHH | |
| DR892 | EVQLQESGGGSVQAGGSLRLSCAASGYSNCSYDMTWYRQAPGKER | 491 |
| | EFVSAIHSDGSTRYADSVKGRFFISQDNAKNTVYLQMNSLKPEDT | |
| | AMYYCKTDPLHCRAHGGSWYSVRANYWGQGTQVTVSSGGSGGSQV | |
| | QLQESGGGSVQAGGSLRLSCAASRYTYNSYCMGWFRQAPGKEREG | |
| | VATIDSDGMTRYADSVKGRFTISKDNAKNTLYLQMNSLKPEDTAM | |
| | YYCAADADCTIAAMTTNPLGQGTQVTVSSASHHHHHH | |
| DR893 | EVQLQESGGGSVQAGGSLRLSCAASGYSNCSYDMTWYRQAPGKER | 492 |
| | EFVSAIHSDGSTRYADSVKGRFFISQDNAKNTVYLQMNSLKPEDT | |
| | AMYYCKTDPLHCRAHGGSWYSVRANYWGQGTQVTVSSGGGSGGGS | |
| | QVQLQESGGGSVQAGGSLRLSCAASRYTYNSYCMGWFRQAPGKER | |
| | EGVATIDSDGMTRYADSVKGRFTISKDNAKNTLYLQMNSLKPEDT | |
| | AMYYCAADADCTIAAMTTNPLGQGTQVTVSSASHHHHHH | |
| DR894 | EVQLQESGGGSVQAGGSLRLSCAASGYSNCSYDMTWYRQAPGKER | 493 |
| | EFVSAIHSDGSTRYADSVKGRFFISQDNAKNTVYLQMNSLKPEDT | |
| | AMYYCKTDPLHCRAHGGSWYSVRANYWGQGTQVTVSSGGSGGSGG | |
| | SGQVQLQESGGGSVQAGGSLRLSCAASRYTYNSYCMGWFRQAPGK | |
| | EREGVATIDSDGMTRYADSVKGRFTISKDNAKNTLYLQMNSLKPE | |
| | DTAMYYCAADADCTIAAMTTNPLGQGTQVTVSSASHHHHHH | |
| DR896 | QVQLQESGGGSVQAGGSLRLSCAASGYSNCSYDMTWYRQAPGKER | 494 |
| | EFVSAIHSDGSTRYADSVKGRFFISQDNAKNTVYLQMNSLKPEDT | |
| | AMYYCKTDPLHCRAHGGSWYSVRANYWGQGTQVTVSSGSQVQLQE | |
| | SGGGSVQAGGSLRLSCAASGYSYSSYCMGWFRQAPGKEREGVATI | |
| | DSDGMTRYADSVKGRFTISKDNAKNTLYLQMNSLKPEDTAMYYCA | |
| | APLYDCDSGAVGRNPPYWGQGTQVTVSSASHHHHHH | |
| DR897 | QVQLQESGGGSVQAGGSLRLSCAASGYSNCSYDMTWYRQAPGKER | 495 |
| | EFVSAIHSDGSTRYADSVKGRFFISQDNAKNTVYLQMNSLKPEDT | |
| | AMYYCKTDPLHCRAHGGSWYSVRANYWGQGTQVTVSSGGSGGSQV | |
| | QLQESGGGSVQAGGSLRLSCAASGYSYSSYCMGWFRQAPGKEREG | |
| | VATIDSDGMTRYADSVKGRFTISKDNAKNTLYLQMNSLKPEDTAM | |
| | YYCAAPLYDCDSGAVGRNPPYWGQGTQVTVSSASHHHHHH | |
| DR898 | QVQLQESGGGSVQAGGSLRLSCAASGYSNCSYDMTWYRQAPGKEREE | 496 |
| | AIHSDGSTRYADSVKGRFFISQDNAKNTVYLQMNSLKPEDTAMYYCK | |
| | PLHCRAHGGSWYSVRANYWGQGTQVTVSSGGGSGGGSQVQLQESGGG | |
| | QAGGSLRLSCAASGYSYSSYCMGWFRQAPGKEREGVATIDSDGMTRY | |
| | SVKGRFTISKDNAKNTLYLQMNSLKPEDTAMYYCAAPLYDCDSGAVG | |
| | PPYWGQGTQVTVSSASHHHHHH | |
| DR899 | QVQLQESGGGSVQAGGSLRLSCAASGYSNCSYDMTWYRQAPGKEREE | 497 |
| | AIHSDGSTRYADSVKGRFFISQDNAKNTVYLQMNSLKPEDTAMYYCK | |
| | PLHCRAHGGSWYSVRANYWGQGTQVTVSSGGSGGSGGSGQVQLQESG | |
| | SVQAGGSLRLSCAASGYSYSSYCMGWFRQAPGKEREGVATIDSDGMT | |
| | ADSVKGRFTISKDNAKNTLYLQMNSLKPEDTAMYYCAAPLYDCDSGA | |
| | RNPPYWGQGTQVTVSSASHHHHHH | |
| DR787 | QVQLQESGGGSVQAGGSLRLSCAASGYSNCSYDMTWYRQAPGKER | 498 |
| | EFVSAIHSDGSTRYADSVKGRFFISQDNAKNTVYLQMNSLKPEDT | |
| | AMYYCKTDPLHCRAHGGSWYSVRANYWGQGTQVTVSSGSQVQLQE | |
| | SGGGSVEAGGSLRLSCAASGYTHSSYCMGWFRQAPGKEREGVAAI | |
| | DVDGSTTYADSVKGRFTISKDNAKNTLYLQMNSLKPEDTGMYYCA | |
| | AEFADCSSNYFLPPGAVRYWGQGTQVTVSSASHHHHHH | |

TABLE 18
IL10Ra/Rb Binding Protein Fc Fusions

| | | SEQ |
|---|---|---|
| Name | Amino Acid Sequence | ID NQ |
| DR900 | QVQLQESGGGSVQAGGSLRLSCAASGYSNCSYDMTWYRQAPGKER | 499 |
| | EFVSAIHSDGSTRYADSVKGRFFISQDNAKNTVYLQMNSLKPEDT | |
| | AMYYCKTDPLHCRAHGGSWYSVRANYWGQGTQVTVSSGGGSQVQL | |
| | QESGGGSVQAGGSLRLSCAASRYTYNSYCMGWFRQAPGKEREGVA | |
| | TIDSDGMTRYADSVKGRFTISKDNAKNTLYLQMNSLKPEDTAMYY | |
| | CAADADCTIAAMTTNPLGQGTQVTVSSASRVESKYGPPCPPCPAP | |
| | EFLGGPSVFLFPPKPKDTLMISRTPEVTCVVVDVSQEDPEVQFNW | |
| | YVDGVEVHNAKTKPREEQFGSTYRVVSVLTVLHQDWLNGKEYKCK | |
| | VSNKGLPSSIEKTISKAKGQPREPQVYTLPPSQEEMTKNQVSLTC | |
| | LVKGFYPSDIAVEWESNGQPENNYKTTPPVLDSDGSFFLYSRLTV | |
| | DKSRWQEGNVFSCSVMHEALHNHYTQKSLSLSLG | |
| DR901 | QVQLQESGGGSVQAGGSLRLSCAASGYSNCSYDMTWYRQAPGKER | 500 |
| | EFVSAIHSDGSTRYADSVKGRFFISQDNAKNTVYLQMNSLKPEDT | |
| | AMYYCKTDPLHCRAHGGSWYSVRANYWGQGTQVTVSSGGGSQVQL | |
| | QESGGGSVQAGGSLRLSCAASGYSYSSYCMGWFRQAPGKEREGVA | |
| | TIDSDGMTRYADSVKGRFTISKDNAKNTLYLQMNSLKPEDTAMYY | |
| | CAAPLYDCDSGAVGRNPPYWGQGTQVTVSSASRVESKYGPPCPPC | |
| | PAPEFLGGPSVFLFPPKPKDTLMISRTPEVTCVVVDVSQEDPEVQ | |
| | FNWYVDGVEVHNAKTKPREEQFGSTYRVVSVLTVLHQDWLNGKEY | |
| | KCKVSNKGLPSSIEKTISKAKGQPREPQVYTLPPSQEEMTKNQVS | |
| | LTCLVKGFYPSDIAVEWESNGQPENNYKTTPPVLDSDGSFFLYSR | |
| | LTVDKSRWQEGNVFSCSVMHEALHNHYTQKSLSLSLG | |
| DR902 | QVQLQESGGGSVQAGGSLRLSCAASGYSNCSYDMTWYRQAPGKER | 501 |
| | EFVSAIHSDGSTRYADSVKGRFFISQDNAKNTVYLQMNSLKPEDT | |
| | AMYYCKTDPLHCRAHGGSWYSVRANYWGQGTQVTVSSGSQVQLQE | |
| | SGGGSVEAGGSLRLSCAASGYTHSSYCMGWFRQAPGKEREGVAAI | |
| | DVDGSTTYADSVKGRFTISKDNAKNTLYLQMNSLKPEDTGMYYCA | |
| | AEFADCSSNYFLPPGAVRYWGQGTQVTVSSASRVESKYGPPCPPC | |
| | PAPEFLGGPSVFLFPPKPKDTLMISRTPEVTCVVVDVSQEDPEVQ | |
| | FNWYVDGVEVHNAKTKPREEQFGSTYRVVSVLTVLHQDWLNGKEY | |
| | KCKVSNKGLPSSIEKTISKAKGQPREPQVYTLPPSQEEMTKNQVS | |
| | LTCLVKGFYPSDIAVEWESNGQPENNYKTTPPVLDSDGSFFLYSR | |
| | LTVDKSRWQEGNVFSCSVMHEALHNHYTQKSLSLSLG | |
| Fc | RVESKYGPPCPPCPAPEFLGGPSVFLFPPKPKDTLMISRTPEVTC | 502 |
| Monomer | VVVDVSQEDPEVQFNWYVDGVEVHNAKTKPREEQFGSTYRVVSVL | |
| | TVLHQDWLNGKEYKCKVSNKGLPSSIEKTISKAKGQPREPQVYTL | |
| | PPSQEEMTKNQVSLTCLVKGFYPSDIAVEWESNGQPENNYKTTPP | |
| | VLDSDGSFFLYSRLTVDKSRWQEGNVFSCSVMHEALHNHYTQKSL | |
| | SLSLG | |

TABLE 30
Nucleic Acid Sequences Encoding Humanized
VHHS of Table 29

| VHH | | |
|---|---|---|
| Encoded | DNA Sequence | SEQ ID |
| DR1223 | GAAGTGCAGTTGCTGGAGAGTGGTGGCGGACTTGTCCAACCCG | 546. |
| | GCGGTAGCCTGCGCCTCAGCTGCGCTGCCAGTGGCTATACACA | |
| | GTGCAGTTATGACATGACCTGGGTCCGCCAAGCGCCCGGCAAG | |
| | GGCCTGGAGTGGGTTTCCGCTATTCACTCCGATGGCAGCACCC | |
| | GTTATGCAGACTCCGTTAAGGGCCGCTTCACCATCTCTCGCGA | |
| | CAACTCCAAGAACACCCTGTATCTTCAGATGAACTCTCTCCGC | |
| | GCTGAGGACACTGCTGTGTATTACTGTAAAACCGATCCACTGC | |
| | ACTGTCGTGCGCACGGGGGCTCATGGTACTCTGTCAGAGCTAA | |
| | CTATTGGGGGCAGGGCACCCTGGTGACTGTGTCCTCT | |
| DR1224 | GAGGTGCAGCTTCTGGAGAGCGGTGGCGGTCTGGTACAACCCG | 547. |
| | GAGGTAGTCTGCGCCTGTCCTGTGCGGCTTCCGGCTACACCCA | |
| | ATGTTCCTATGACATGACCTGGGTTCGCCAGGCCCCCGGTAAG | |
| | GGCCTGGAATTTGTCAGCGCTATCCATAGTGATGGAAGCACCA | |
| | GATATGCCGACAGTGTTAAGGGCCGCTTCACCATCAGCAGAGA | |
| | TAATAGCAAGAACACCCTTTACCTCCAGATGAACTCTTTGCGT | |
| | GCCGAGGATACCGCCGTCTATTACTGTAAGACCGATCCGCTGC | |
| | ATTGCCGTGCACATGGCGGGTCCTGGTACAGTGTTCGCGCCAA | |
| | CTATTGGGGCCAGGGAACACTGGTGACTGTCAGCAGT | |
| DR1225 | GAAGTGCAACTGGTGGAGAGCGGTGGAGGTTTGGTTCAGCCTG | 548. |
| | GAGGCTCCCTGCGCCTTTCCTGTGCTGCGAGCGGGTATACACA | |
| | GTGCAGCTATGACATGACCTGGGTGAGGCAGGCACCGGGCAAA | |
| | GGTTTGGAGTGGGTGTCAGCGATCCATTCTGATGGCAGCACAA | |
| | GGTACGCGGATAGCGTTAAGGGCAGGTTTTTCATTTCCCGCGA | |
| | CAACTCTAAGAACACACTCTATCTCCAGATGAACTCACTTCGC | |
| | GCCGAGGATACCGCCGTGTATTACTGCAAGACAGACCCTCTGC | |
| | ACTGTCGGGCTCACGGCGGTAGCTGGTATTCCGTCCGTGCTAA | |
| | CTACTGGGGCCAGGGCACTCTCGTGACCGTGTCATCC | |
| DR1226 | GAGGTGCAGCTGGTGGAGTCTGGGGGAGGCCTGGTACAGCCTG | 549. |
| | GGGGCAGTCTGCGCCTGAGCTGCGCGGCCAGTGGCTATACTCA | |
| | GTGCTCCTACGACATGACTTGGGTAAGGCAGGCCCCCGGCAAA | |
| | GGTTTGGAGTTTGTTTCCGCAATTCATAGCGACGGCTCCACAC | |
| | GCTACGCCGATTCCGTCAAAGGTCGGTTTTTCATCTCCCGTGA | |
| | TAACAGCAAAAACACTCTGTACCTTCAGATGAACTCCCTCCGC | |
| | GCAGAGGATACCGCCGTTTATTACTGTAAAACTGACCCCCTCC | |
| | ACTGCCGCGCTCACGGCGGGAGCTGGTATAGTGTTCGCGCCAA | |
| | CTACTGGGGCCAGGGCACCTTGGTGACCGTCTCCAGT | |
| DR1227 | GAGGTGCAGTTGCTGGAAAGCGGGGGTGGCCTGGTCCAGCCGG | 550. |
| | GAGGTTCCCTGCGCCTTTCCTGCGCCGCTTCCGGCTACACCCA | |
| | TTCAAGTTACTGTATGGGCTGGGTTCGTCAGGCACCTGGCAAA | |
| | GGTCTCGAATGGGTGAGCGCTATCGACGTGGACGGAAGCACCA | |
| | CTTATGCTGATTCCGTGAAGGGTAGGTTCACCATTTCCCGCGA | |
| | CAACTCCAAGAACACTCTTTATCTGCAAATGAATAGCCTCCGC | |
| | GCTGAGGACACCGCCGTATATTACTGCGCTGCGGAGTTTGCCG | |
| | ACTGCTCTAGCAACTACTTTCTGCCCCCTGGGGCCGTAAGGTA | |
| | TTGGGGCCAGGGCACCTTGGTGACTGTGTCCTCC | |
| DR1228 | GAGGTGCAGCTGCTCGAATCCGGGGGCGGACTGGTGCAGCCTG | 551. |
| | GTGGCAGCCTGCGTCTGTCCTGTGCTGCCTCTGGTTATACCCA | |
| | TTCCAGCTACTGTATGGGTTGGGTGCGCCAAGCCCCTGGAAAG | |
| | GGGCTTGAAGGCGTCTCCGCCATTGATGTCGATGGCAGTACCA | |
| | CTTACGCTGACTCCGTGAAGGGACGCTTCACTATCAGCCGCGA | |
| | CAACTCCAAGAACACGCTCTATCTCCAGATGAACTCTCTCCGC | |
| | GCAGAAGACACCGCAGTGTACTATTGTGCCGCAGAGTTTGCCG | |
| | ATTGCAGCTCAAACTACTTCCTGCCCCCAGGGGCAGTGCGTTA | |
| | TTGGGGCCAGGGAACACTTGTTACTGTTTCTTCT | |
| DR1229 | GAGGTTCAGTTGGTGGAATCTGGCGGGGGCCTCGTGCAGCCCG | 552. |
| | GAGGCTCCCTCAGGCTTTCCTGTGCTGCCTCTGGGTACACACA | |
| | CTCTAGTTATTGCATGGGCTGGGTGCGCCAAGCACCGGGCAAG | |
| | GGTCTGGAGTGGGTGAGCGCCATTGATGTGGACGGTTCTACTA | |
| | CCTATGCCGATAGCGTGAAGGGGCGTTTCACAATCAGCCGCGA | |
| | CAACTCCAAAAACACCCTGTATCTCCAGATGAATAGTCTTCGT | |
| | GCCGAAGACACCGCAGTGTACTATTGTGCGGCAGAGTTCGCGG | |
| | ACTGCTCTAGCAACTATTTTCTGCCCCCAGGAGCCGTCCGCTA | |
| | CTGGGGACAGGGCACCCTGGTCACCGTGTCTTCC | |
| DR1230 | GAGGTCCAGCTGGTGGAGAGTGGGGGCGGACTGGTACAGCCAG | 553. |
| | GTGGCAGTCTTCGCCTCTCTTGCGCTGCCTCCGGCTACACTCA | |
| | CAGCTCTTATTGCATGGGTTGGGTGAGACAGGCCCCTGGTAAA | |
| | GGTCTTGAGGGCGTGAGTGCCATCGACGTGGATGGTTCCACTA | |
| | CCTACGCTGATAGTGTGAAGGGCCGCTTCACTATCTCACGTGA | |
| | TAACTCTAAGAATACTCTGTATCTTCAAATGAACAGCCTCCGC | |
| | GCAGAGGACACCGCAGTCTACTATTGTGCAGCCGAGTTTGCTG | |
| | ACTGTAGCTCCAACTACTTCCTTCCGCCCGGTGCAGTGCGCTA | |
| | TTGGGGGCAAGGCACTCTGGTGACAGTCTCCTCC | |
| DR1308 | GAGGTGCAGCTGGTTGAATCCGGGGGTGGCCTCGTGCAGCCCG | 554. |
| | GAGGCTCTCTGCGCCTGTCCTGCGCAGCCTCCGGCTATACCCA | |
| | GTGTTCCTATGATATGACGTGGGTCAGACAAGCTCCTGGCAAA | |
| | GGCCGCGAGTTTGTTTCCGCTATTCACTCTGACGGCTCCACCC | |
| | GGTACGCGGATAGCGTTAAGGGTCGCTTTTTCATCAGCCGGGA | |
| | TAACTCAAAGAACACTCTGTACTTGCAGATGAACTCTCTTCGC | |
| | GCTGAGGATACGGCGGTTTATTACTGCAAAACTGATCCTCTCC | |
| | ACTGTCGCGCCCACGGCGGATCTTGGTATAGCGTTCGCGCTAA | |
| | TTACTGGGGGCAAGGAACTCTGGTGACTGTTTCCTCC | |
| DR1309 | GAGGTGCAGTTGCAGGAGTCCGGCGGAGGGTCCGTCCAGGCCG | 555. |
| | GAGGCAGTCTGCGCCTGTCCTGTGCCGCTTCCGGCTACTCCCA | |
| | ATGTTCTTACGACATGACTTGGTATCGCCAGGCTCCGGGCAAG | |
| | GAGCGCGAGTTCGTGAGCGCCATCCACTCAGACGGCTCCACCC | |
| | GTTACGCCGACTCAGTCAAGGGTCGCTTCTTTATCTCCCAGGA | |
| | CAACGCAAAGAACACCGTCTATCTCCAGATGAACTCCCTGAAG | |
| | CCAGAAGACACAGCTATGTATTACTGTAAAACCGACCCACTGC | |
| | ATTGCAGGGCTCATGGTGGCTCCTGGTACTCTGTACGCGCAAA | |
| | CTACTGGGGTCAGGGCACCCAGGTGACCGTGAGTTCC | |

TABLE 31
Nucleic Acid Sequences Encoding Humanized IL10R
Binding Molecules of Table 29

| VHH | | |
|---|---|---|
| Encoded | DNA Sequence | SEQ ID |
| DR1328 | GAAGTCCAGCTTGTTGAATCTGGGGGAGGCCTGGTGCAGCCTG | 556 |
| | GGGGCTCCCTGCGCCTCTCCTGTGCTGCCAGCGGGTACACCCA | |
| | ATGTTCCTATGATATGACCTGGGTCCGCCAGGCACCAGGCAAG | |
| | GGATTGGAATTTGTGAGCGCAATCCATTCTGACGGCAGTACTC | |
| | GTTACGCTGACTCCGTAAAGGGCAGATTCTTTATCTCTCGTGA | |
| | CAACAGCAAAAACACTCTGTATCTGCAAATGAACTCTCTCCGC | |
| | GCCGAAGACACTGCGGTCTATTACTGCAAGACCGATCCCCTCC | |
| | ATTGTCGTGCCCACGGGGGAAGTTGGTACTCAGTCCGCGCAAA | |
| | TTATTGGGGCCAGGGCACTCTCGTGACCGTCTCCAGCGGGGGA | |
| | GGTTCCGAGGTGCAGCTCGTGGAGAGCGGTGGGGGCCTGGTCC | |
| | AGCCTGGCGGAAGCCTGCGCCTGTCATGCGCTGCGTCTGGTTA | |
| | CACACACAGCTCCTATTGTATGGGATGGGTGCGGCAGGCCCCC | |
| | GGTAAAGGACTGGAATGGGTTAGCGCCATCGACGTGGACGGCT | |
| | CTACGACATACGCCGACTCCGTCAAGGGCCGCTTTACTATCAG | |
| | CCGTGATAACAGCAAGAACACTCTGTACCTGCAAATGAACTCT | |
| | CTCCGCGCCGAGGACACCGCTGTGTATTACTGTGCCGCAGAGT | |
| | TCGCAGACTGCTCTTCCAATTATTTCCTGCCTCCCGGAGCCGT | |
| | CCGTTATTGGGGTCAGGGCACTCTGGTTACTGTGTCCTCTGCT | |
| | TCCCATCACCATCATCACCAC | |
| DR1329 | GAGGTGCAGCTGGTTGAGTCTGGCGGAGGTCTGGTGCAGCCCG | 557 |
| | GCGGTTCTCTGCGCCTGTCTTGCGCCGCGAGCGGCTACACGCA | |
| | ATGCAGCTACGATATGACCTGGGTTCGTCAGGCCCCCGGCAAG | |
| | GGCTTGGAGTTCGTCAGCGCGATCCATAGCGACGGCTCCACAA | |
| | GATATGCTGATAGCGTGAAGGGCCGCTTCTTTATTAGTAGAGA | |
| | CAACTCTAAGAACACACTCTACCTCCAGATGAATAGTCTCCGC | |
| | GCAGAGGACACGGCGGTGTACTATTGTAAGACCGATCCTCTGC | |
| | ATTGCAGAGCCCACGGTGGCTCCTGGTACAGCGTGCGGGCTAA | |
| | CTATTGGGGGCAAGGCACCCTGGTTACTGTCTCTAGCGGTGGA | |
| | GGTAGCGAAGTCCAACTTGTCGAGTCCGGGGGAGGTCTGGTCC | |
| | AGCCCGGTGGGTCTCTTAGGCTCTCCTGCGCTGCATCCGGCTA | |
| | CACCCATAGCTCATATTGTATGGGCTGGGTCCGCCAAGCCCCT | |
| | GGGAAGGGGCTGGAAGGTGTCAGCGCCATTGATGTCGATGGCA | |
| | GCACAACTTACGCTGATAGTGTGAAGGGTCGCTTCACTATCTC | |
| | CAGAGACAATTCAAAGAACACCTTGTACCTCCAGATGAACTCT | |
| | CTTCGCGCGGAAGACACCGCCGTCTATTACTGCGCCGCTGAGT | |
| | TCGCGGACTGTAGCTCCAATTATTTCCTGCCACCCGGCGCTGT | |
| | GCGGTATTGGGGCCAGGGTACTCTGGTGACTGTGTCTAGTGCC | |
| | TCTCACCATCACCATCATCAC | |
| DR1330 | GAGGTGCAGCTGGTGGAATCTGGAGGCGGTCTCGTCCAGCCAG | 558 |
| | GGGGCTCTCTGCGTCTGTCTTGCGCTGCGTCTGGATATACCCA | |
| | GTGCTCTTACGACATGACATGGTATCGGCAGGCTCCCGGCAAG | |
| | GGACTTGAGTTCGTCTCTGCCATCCACAGCGATGGTTCCACAC | |
| | GCTACGCAGATTCCGTGAAGGGACGTTTTTTCATCAGCCGTGA | |
| | CAATAGCAAGAACACTCTGTATCTTCAAATGAACTCCCTCCGC | |
| | GCGGAAGACACCGCCGTGTATTACTGTAAAACCGATCCTCTGC | |
| | ACTGTAGGGCACATGGGGGCTCTTGGTATTCTGTTCGTGCTAA | |
| | TTATTGGGGCCAGGGCACCCTGGTCACCGTTTCCAGCGGCGGA | |
| | GGTAGTGAGGTCCAGCTCGTCGAATCCGGCGGTGGCCTCGTGC | |
| | AGCCTGGCGGGTCCCTCCGCCTGTCCTGCGCTGCAAGCGGTTA | |
| | TACGCACAGCTCCTATTGCATGGGCTGGGTGCGTCAAGCTCCT | |
| | GGCAAAGGCCTGGAGTGGGTCAGCGCTATTGACGTGGACGGGA | |
| | GTACTACCTACGCCGATTCAGTGAAGGGTAGATTCACCATCTC | |
| | ACGTGACAACTCTAAGAACACACTGTACTTGCAGATGAATAGC | |
| | CTGAGGGCTGAAGACACTGCTGTGTACTATTGCGCTGCCGAGT | |
| | TCGCCGATTGTAGCTCCAACTACTTCCTGCCCCCAGGAGCGGT | |
| | CCGTTACTGGGGCCAAGGCACCCTCGTGACTGTCTCTTCAGCC | |
| | AGTCATCACCATCATCACCAC | |
| DR1331 | GAGGTCCAGCTGGTGGAGAGTGGGGGTGGGCTCGTTCAGCCTG | 559 |
| | GGGGCTCTCTGAGGCTGAGCTGTGCAGCCTCCGGCTATACCCA | |
| | GTGTTCTTACGACATGACGTGGTATAGGCAGGCACCAGGCAAA | |
| | GGACTGGAGTTTGTGAGTGCTATCCACTCCGACGGTTCCACCC | |
| | GGTATGCTGACTCTGTTAAGGGCAGATTCTTTATTAGCCGGGA | |
| | CAACAGCAAAAACACCCTTTACCTCCAGATGAACTCTCTGAGG | |
| | GCAGAGGACACGGCAGTGTATTACTGTAAAACAGACCCTCTTC | |
| | ACTGCCGCGCACATGGAGGCTCTTGGTACAGTGTGAGGGCTAA | |
| | TTATTGGGGTCAGGGCACCCTCGTCACAGTCTCTTCAGGGGGT | |
| | GGATCTGAGGTGCAGTTGGTCGAGAGTGGGGGTGGCCTGGTGC | |
| | AGCCTGGCGGTAGCCTGCGCCTGAGCTGCGCGGCCTCTGGGTA | |
| | CACCCACTCCTCTTACTGTATGGGCTGGGTGAGGCAGGCTCCT | |
| | GGAAAGGGGCTGGAGGGCGTGTCCGCTATTGACGTAGATGGCT | |
| | CCACTACCTACGCCGACAGCGTAAAAGGTCGTTTCACAATCTC | |
| | CCGCGACAACTCCAAGAACACCCTCTACTTGCAGATGAACTCC | |
| | CTGCGGGCCGAAGACACAGCTGTTTATTACTGCGCCGCTGAGT | |
| | TCGCAGACTGTTCCAGTAATTACTTCCTGCCCCCTGGTGCCGT | |
| | GCGTTACTGGGGCCAGGGCACCCTCGTAACCGTCAGCTCCGCT | |
| | AGTCACCATCACCACCACCAT | |

### EXAMPLES

### Example 1-VₕH Generation

Camels were acclimated at research facility for at least 7 days before immunization. Antigen was diluted with 1×PBS (antigen total about 1 mg). The quality of the antigen was assessed by SDS-PAGE to ensure purity (e.g., >80%). For the first time, 10 mL CFA (then followed 6 times using IFA) was added into mortar, then 10 mL antigen in 1×PBS was slowly added into the mortar with the pestle grinding. The antigen and CFA/IFA were ground until the component showed milky white color and appeared hard to disperse. Camels were injected with antigen emulsified in CFA subcutaneously at at least six sites on the body, injecting about 2 mL at each site (total of 10 mL per camel). A stronger immune response was generated by injecting more sites and in larger volumes. The immunization was conducted every week (7 days), for 7 times. The needle was inserted into the subcutaneous space for 10 to 15 seconds after each injection to avoid leakage of the emulsion. Alternatively, a light pull on the syringe plunger also prevented leakage. The blood sample was collected three days later after 7th immunization.

To generate VHHs against hIL10Ra, the immunogen employed was the extracellular domain (amino acids 22-235) of hIL10Ra (UNIPROT Ref: Q13651). To generate VHHs against hIL10Rb, the immunogen employed was the 201 amino acid extracellular domain of the hIL10Rb, amino acids 20-220 of the precursor and amino acids 1-201 of the mature protein (UNIPROT Reference No. Q08334). To generate VHHs against mIL10Rb, the immunogen employed was the 201 amino acid extracellular domain of the mIL10Rb, amino acids 20-220 of the precursor and amino acids 1-201 of the mature protein (UNIPROT Reference No. Q61190). With respect to each antigen, the following methodology was used to identify and isolate the VHHs.

After immunization, the library was constructed. Briefly, RNA was extracted from blood and transcribed to cDNA. The VHH regions were obtained via two-step PCR, which fragment about 400 bp. The PCR outcomes and the vector of pMECS phagemid were digested with Pst I and Not I, subsequently, ligated to pMECS/Nb recombinant. After ligation, the products were transformed into Escherichia coli (E. coli) TGI cells by electroporation. Then, the transformants were enriched in growth medium and planted on plates. Finally, the library size was estimated by counting the number of colonies. Library biopanning was conducted to screen candidates against the antigens after library construction. Phage display technology was applied in this procedure. Positive colonies were identified by PE-ELISA.

### Example 2. Generation of Anti-hIL10R VHHs

Camels were immunized with the extracellular domains of the human IL10R (amino acids 22-235, UniProtKB Q13651, hIL-10Rαecd) and IL10Rβ (amino acids 20-220, UniProtKB Q08334, hIL-10Rαecd) weekly for seven weeks and PBMCs harvested on day 52. Phage display libraries were constructed and biopanning conducted as described in Example 1 above. 50 VHH sequences were obtained after selection on hIL10-R1 and 47 VHH sequences were obtained after selection on hIL10-R2. Sequences were clonotyped using germline assignment and CDR3 sequence similarity.

### Example 3. Synthesis of DNA Encoding Synthekines

Seven unique anti-hIL-10Rα_{ecd} sequences (SEQ ID Nos: 44-50) and seven unique anti-hIL-10Rβ_{ecd} sequences (SEQ ID Nos: 51-57) were selected from each cohort and DNA was synthesized consisting of one IL-10R VHH encoding DNA and one IL-10RαVHH encoding DNA separated by a linker sequence by GGGS (SEQ ID NO: 484) encoding DNA. DNA was for each possible VHH combination and in both orientations for a total of 98 7×7×2=98 VHH dimers. An Ala-Ser (“AS”) linker followed by His-6 (SEQ ID NO: 532) DNA (ASH6, SEQ ID NO: 560) was added at the 3′ end of each DNA construct. The codon optimized DNA sequences encoding these constructs are provided as SEQ ID Nos: 290-237 and the orientation of components thereof are described in Table 2 of the specification above.

### Example 4. Recombinant Production and Purification

Codon optimized DNA inserts (SEQ ID Nos: 290-237) and cloned into modified pcDNA3.4 (Genscript) for small scale expression in HEK293 cells in 24 well plates. Supernatants The cells The IL2R binding proteins were purified in substantial accordance with the following procedure. Using a Hamilton Star automated system, 96×4 ml of supernatants in 4×24-well blocks were re-arrayed into 4×96-well, 1 mL blocks. PhyNexus micropipette tips (Biotage, San Jose CA) holding 80 uL of Ni-Excel IMAC resin (Cytiva) are equilibrated wash buffer: PBS pH 7.4, 30 mM imidazole. PhyNexus tips were dipped and cycled through 14 cycles of 1 mL pipetting across all 4×96-well blocks. PhyNexus tips were washed in 2×1 mL blocks holding wash buffer. PhyNexus tips were eluted in 3×0.36 mL blocks holding elution buffer: PBS pH 7.4, 400 mM Imidazole. PhyNexus tips were regenerated in 3×1 mL blocks of 0.5 M sodium hydroxide.

The purified protein eluates were quantified using a Biacore® T200 as in substantial accordance with the following procedure. 10 uL of the first 96×0.36 mL eluates were transferred to a Biacore® 96-well microplate and diluted to 60 uL in HBS-EP+ buffer (10 mM Hepes pH 7.4, 150 mM NaCl, 1 mM EDTA, 0.05% Tween 20). Each of the 96 samples was injected on a CM5 series S chip previously functionalized with anti-histidine capture antibody (Cytiva): injection is performed for 18 seconds at 5 uL/min. Capture levels were recorded 60 seconds after buffer wash. A standard curve of known VHH concentrations (270, 90, 30, 10, 3.3, 1.1 μg/mL) was acquired in each of the 4 Biacore chip flow cells to eliminate cell-to-cell surface variability. The 96 captures were interpolated against the standard curve using a non-linear model including specific and unspecific, one-site binding. Concentrations in the first elution block varied from 12 to 452 μg/mL corresponding to a 4-149 μg. SDS-PAGE analysis of 5 randomly picked samples was performed to ensure molecular weight of eluates corresponded to expected values (˜30 KDa).

The concentration of the proteins was normalized using the Hamilton Star automated system in substantial accordance with the following procedure. Concentration values are imported in an Excel spreadsheet where pipetting volumes were calculated to perform dilution to 50 μg/mL in 0.22 mL. The spreadsheet was imported in a Hamilton Star method dedicated to performing dilution pipetting using the first elution block and elution buffer as diluent. The final, normalized plate was sterile filtered using 0.22 μm filter plates (Corning) and the material used for the following in vitro assays.

### Example 5. Evaluation of Binding Affinity Via Surface Plasmon Resonance

One representative example from each clonotype of IL10a generated in accordance with Examples 1-3 was selected for evaluation of binding via SPR as follows. Evaluation of binding affinity of the IL10Ra binding molecules for the ECD of hIL10Ra corresponding to SEQ ID NOS 159, 161, 162, 163, 165, 167 and 170 was conducted using surface plasmon resonance (SPR) in substantial accordance with the following procedure. All experiments were conducted in 10 mM Hepes, 150 mM NaCl, 0.05% (v/v) Polysorbate 20 (PS20) and 3 mM EDTA (HBS-EP+ buffer) on a Biacore T200 instrument equipped with a Protein A derivatized sensor chip (Cytiva). Mono-Fc VHH ligands were flowed at 5 μl/min for variable time ranging from 18 to 300 seconds, reaching the capture loads listed in the tables below. Following ligand capture, injections of a 2-fold dilution series of the extracellular domain of the IL2Rb-receptor modified to incorporate a C-terminal poly-His sequence, typically comprising at least five concentrations between 1 μM and 1 nM, were performed in either high performance or single cycle kinetics mode. Surface regeneration was achieved by flowing 10 mM glycine-HCl, pH 1.5 (60 seconds, 50 μL/min). Buffer-subtracted sensograms were processed with Biacore T200 Evaluation Software and globally fit with a 1:1 Langmuir binding model (bulk shift set to zero) to extract kinetics and affinity constants (kₐ, k_{d}, K_{D}). R_{MAX}<100 RU indicates surface density compatible with kinetics analysis. Calculated Rₘₐₓ values were generated using the equation: Rmax=Load (RU)×valency of ligand×(Molecular weight of analyte/Molecular weight of ligand). Surface activity was defined as the ratio of experimental/calculated Rmax. The results of these binding affinity experiments are provided in Tables 23, 24 and 25.

### Example 6. IL10 Activity Assay

HEK-Blue™ IL-10 reporter cell line (Invivogen, San Diego CA) was used for screening the IL10R1/R2 VHHs. HEK-Blue™ IL-10 cells were generated by stable transfection of the human embryonic kidney HEK293 cell line with the genes encoding hIL-10R α and β chains, human STAT3, and the STAT3-inducible SEAP (secreted embryonic alkaline phosphatase) reporter. Binding of IL-10 to its receptor on the surface of HEK-Blue™ IL-10 cells triggers JAK1/STAT3 signaling and the subsequent production of SEAP. The signal was then detected by quantifying SEAP activity in the cell culture supernatant using a QUANTI-Blue™ development solution (Invivogen, San Diego CA) and the absorbance values were measured spectrophotometrically at 630 nm. Because STAT3 is also implicated in the signaling of cytokines such as IFN-α/β and IL-6, HEK-Blue™ IL-10 cells are knockout for the expression of hIFNAR2 and hIL-6R.

### Example 7. Screening of SEQ ID NOs: 192-289

To screen the IL10R1/R2 VHHs, HEK-Blue™ IL-10 cells were seeded in a 96-well plate at 50,000 cells per well and treated with either 25 nM or 100 nM protein (in triplicates) for 24 hours. Recombinant Animal-Free Human IL-10 (Shenandoah Biotechnology, Inc. Warwick, PA Catalog No. 100-83AF) was used as a positive control and unstimulated cells were used as a negative control. 24 hours post treatment, 20 μl of the cell supernatant was transferred to a flat-bottom 96 well plate and the assay was developed by adding 180 μl of the QUANTI-Blue™ (Invivogen) for 2 hours. The absorbance values were measured at 630 nm on the Envision® (PerkinElmer, Waltham MA) multilabel plate reader. The results of this screening are presented in Table 3 of the specification.

### Example 8. Evaluation of Binding Affinity of IL10Ra and IL10Rb Single Domain Antibodies Via Surface Plasmon Resonance

One representative example from each clonotype generated in accordance with Examples 1-3 was selected for evaluation of binding via SPR as follows. Evaluation of binding affinity of the IL2Rb binding molecules for CD122 corresponding to SEQ ID NOS 21, 29, 33, 37, 45, 53 and 65 was conducted using surface plasmon resonance (SPR) in substantial accordance with the following procedure. All experiments were conducted in 10 mM Hepes, 150 mM NaCl, 0.05% (v/v) Polysorbate 20 (PS20) and 3 mM EDTA (HBS-EP+ buffer) on a Biacore T200 instrument equipped with a Protein A derivatized sensor chip (Cytiva). Mono-Fc VHH ligands were flowed at 5 μl/min for variable time ranging from 18 to 300 seconds, reaching the capture loads listed in the tables below. Following ligand capture, injections of a 2-fold dilution series of the extracellular domain of the IL2Rb-receptor modified to incorporate a C-terminal poly-His sequence, typically comprising at least five concentrations between 1 μM and 1 nM, were performed in either high performance or single cycle kinetics mode. Surface regeneration was achieved by flowing 10 mM glycine-HCl, pH 1.5 (60 seconds, 50 μL/min). Buffer-subtracted sensograms were processed with Biacore T200 Evaluation Software and globally fit with a 1:1 Langmuir binding model (bulk shift set to zero) to extract kinetics and affinity constants (kₐ, k_{d}, K_{D}). R_{MAX}<100 RU indicates surface density compatible with kinetics analysis. Calculated Rₘₐₓ values were generated using the equation: Rₘₐₓ=Load (RU)×valency of ligand×(Molecular weight of analyte/Molecular weight of ligand). Surface activity was defined as the ratio of experimental/calculated Rmax. The results of these binding affinity experiments are provided in Table 4 below.

### Example 9. Evaluation of Binding Affinity IL10R Binding Molecules Via Surface Plasmon Resonance

All experiments were conducted in 10 mM Hepes, 150 mM NaCl, 0.05% (v/v) Polysorbate 20 (PS20) and 3 mM EDTA (HBS-EP+ buffer) on a Biacore T200 instrument equipped with Protein A or CAP biotin chips (Cytiva). For experiments on Protein A chips, Fc-fused ligands were flowed at 5 μl/min for variable time ranging from 18 to 300 seconds, reaching the capture loads listed in the tables below.

Following ligand capture, injections of a 2-fold dilution series of analyte typically comprising at least five concentrations between 1 μM and 1 nM were performed in either high performance or single cycle kinetics mode. Surface regeneration was achieved by flowing 10 mM glycine-HCl, pH 1.5 (60 seconds, 50 μL/min). Buffer-subtracted sensograms were processed with Biacore T200 Evaluation Software and globally fit with a 1:1 Langmuir binding model (bulk shift set to zero) to extract kinetics and affinity constants (kₐ, k_{d}, K_{D}). R_{MAX}<100 RU indicates surface density compatible with kinetics analysis.

Experiments on CAP chips were performed as described above with an additional capture step of Biotin CAPture reagent (10 seconds, 40 uL/min) performed prior to capture of biotinylated ligands.

Calculated Rmax were generated using the equation Rmax=Load (RU)×valency of ligand×(Molecular weight of analyte/Molecular weight of ligand. Surface activity was defined as the ratio experimental/calculated Rmax. The configurations for samples evaluated and the results of these experiments are provided in Table s 22 and 23

### Example 10: Evaluation of Binding Affinity of Humanized IL10RA Binding Molecules Via Surface Plasmon Resonance

All experiments were conducted in 10 mM Hepes, 150 mM NaCl, 0.05% (v/v) Polysorbate 20 (PS20) and 3 mM EDTA (HBS-EP+ buffer) on a Biacore T200 instrument equipped with an anti-histidine capture chip (Cytiva). ˜0.5 μg/mL hIL10Rb-his (Sino Biological cat #10945) was flowed at 5 μl/min for 120 seconds, reaching the capture loads listed in the table below. Following ligand capture, injections of a 2-fold dilution series (50, 25, 12.5, 6.25, 3.13 nM) of humanized, mono-Fc VHHs (see analyte in table) was performed in single cycle kinetics mode. Surface regeneration was achieved by flowing 10 mM glycine-HCl, pH 1.5 (60 seconds, 50 μL/min). Buffer-subtracted sensograms were processed with Biacore T200 Evaluation Software and globally fit with a 1:1 Langmuir binding model (bulk shift set to zero) to extract kinetics and affinity constants (kₐ, k_{d}, K_{D}). R_{MAX}<100 RU indicates surface density compatible with kinetics analysis. Calculated Rmax were generated using the equation Rmax=Load (RU)×valency of ligand×(Molecular weight of analyte/Molecular weight of ligand. Surface activity was defined as the ratio experimental/calculated Rmax. See tables below for sample information and experimental results.

1. IL10 receptor (IL10R) binding molecule comprising:
(a) a first single domain antibody (sdAb) that specifically binds to the extracellular domain a first subunit of a IL10Ra (IL10Ra sdAb); and
(b) a second single domain antibody that specifically binds to extracellular domain of a IL10Rb (IL10Rb sdAb);
wherein:
contacting a cell expressing the IL10Ra and IL10Rb with an effective amount of the IL10R binding molecule results in intracellular signaling.

2. The IL10R binding molecule of claim 1 wherein the IL10Ra sdAb and IL10Rb are covalently linked.

3. The IL10R binding molecule of claim 1 wherein the IL10Ra sdAb and IL10Rb are covalently linked using a linker.

4. The IL10R binding molecule of claim 1 wherein the IL10 receptor binding molecule is a single polypeptide.

5. The IL10R binding molecule IL10R is a polypeptide of the following formula [#1]:
H2N-(IL10 VHH #1)-(L1)ₐ-(IL10 VHH #2)-(L2)_{b}-(CP)_{c}-COOH [#1]
wherein: “-” represents a covalent bond; L1 and L2 are linkers; CP is a chelating peptide; a, b, and c are independently selected from the integers 0 or 1; “H2N” denotes the amino terminus; and “COOH” denotes the carboxy terminus of the polypeptide.

6. The IL10R binding molecule of claim 5 wherein the IL10 VHH #1 is an IL10Ra sdAb and IL10 VHH #2 is an IL10Rb sdAb, IL10 VHH #1 is an IL10Rb sdAb and IL10 VHH #2 is an IL10Ra sdAb.

7. The IL10R binding molecule of claim 6 wherein IL10Ra sdAb is an sdAb comprising:
a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 1, 4, 7, 10, 13, 16, 19, 22, 25, 28, 31, 34, 37, 40, 43, 46, 49, and 52;
a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 2, 5, 8, 11, 14, 17, 20, 23, 26, 29, 32, 35, 38, 41, 44, 47, 50 and 53; and
a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 3, 6, 9, 12, 15, 18, 21, 24, 27, 30, 33, 36, 39, 42, 45, 48, 51 and 54;
and the IL10Rb sdAb is an sdAb comprising:
a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148 and 151;
a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149 and 152; and
a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150 and 153.

8. The IL10R binding molecule of claim 6 wherein L10Ra sdAb is an sdAb having least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative to any one of SEQ ID NOS: 154-171.

9. The IL10R binding molecule of claim 6 wherein IL10Rb sdAb is an sdAb having least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative to any one of SEQ ID NOS: 172-198 or SEQ ID Nos: 199-201.

10. The IL10R binding molecule of claim 6 wherein having least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative to any one of SEQ ID NOS: 256-353.

11. The IL10R binding molecule of claim 6 wherein having least 90% % (e.g., 91%, 92%, 93%, 94%, 95%, 96%, 97%, 98%, 99%, or 100%) sequence identity, or having 0, 1, 2, or 3 amino acid changes, optionally conservative amino acid changes relative to any one of SEQ ID NOS: 172-198 or SEQ ID Nos: 199-201.

12. The IL10R binding molecule [ ] of claim 6 wherein having least 90% (e.g., 91%, 92%, 93%, 94%, 95%, 96%, 97%, 98%, 99%, or 100%) sequence identity to the sequence of any one of the IL10R binding molecules of Table 29 (SEQ ID NOS: [DR1511-DR1525]).

13. The IL10R binding molecule of claim 6 wherein the affinity of the IL10Ra sdAb has a higher affinity for the extracellular domain of IL10Ra than the affinity of the IL10Rb sdAb for the extracellular domain of IL10Rb.

14. The IL10R binding molecule of claim 1, wherein the IL10R binding protein is PEGylated

15. A pharmaceutically acceptable formulation of an IL10R binding molecule of claim 1.

16. A nucleic acid sequence encoding the IL10R binding molecule of claim 6.

17. A recombinant vector comprising the nucleic acid of claim 16.

18. A method of treating a mammalian subject suffering from an autoimmune disease, infectious disease, or inflammatory disease by the administration of a therapeutically effective amount of an IL10R binding molecule of claim 1.

19. A method of treating a mammalian subject suffering from a neoplastic disease by the administration of a therapeutically effective amount of an IL10R binding molecule of claim 1.

20. A cell comprising the nucleic acid of claim 16.

21. A method of treating a mammalian subject suffering from an autoimmune disease, infectious disease, or inflammatory disease by the administration of a therapeutically effective amount of the pharmaceutically acceptable formulation of claim 15.

22. A method of treating a mammalian subject suffering from an autoimmune disease, infectious disease, or inflammatory disease by the administration of a therapeutically effective amount of the nucleic acid of claim 16.

23. A method of treating a mammalian subject suffering from an autoimmune disease, infectious disease, or inflammatory disease by the administration of a therapeutically effective amount of the recombinant vector of claim 17.

24. A method of treating a mammalian subject suffering from an autoimmune disease, infectious disease, or inflammatory disease by the administration of a therapeutically effective amount of the cell of claim 20.

25. A method of treating a mammalian subject suffering from a neoplastic disease by the administration of a therapeutically effective amount of the pharmaceutically acceptable formulation of claim 15.

26. A method of treating a mammalian subject suffering from a neoplastic disease by the administration of a therapeutically effective amount of the nucleic acid of claim 16.

27. A method of treating a mammalian subject suffering from a neoplastic disease by the administration of a therapeutically effective amount of the recombinant vector of claim 17.

28. A method of treating a mammalian subject suffering from a neoplastic disease by the administration of a therapeutically effective amount of the cell of claim 20.


SEQUENCE LISTING (569 sequences)

SEQ ID NO: 1 - protein, 9 aa, synthetic construct
  source 1..9: note = Description of Artificial Sequence: Synthetic peptide
YLYSIDYMA

SEQ ID NO: 2 - protein, 17 aa, synthetic construct
  source 1..17: note = Description of Artificial Sequence: Synthetic peptide
VIYTASGATFYPDSVKG

SEQ ID NO: 3 - protein, 16 aa, synthetic construct
  source 1..16: note = Description of Artificial Sequence: Synthetic peptide
VRKTDSYLFDAQSFTY

SEQ ID NO: 4 - protein, 9 aa, synthetic construct
  source 1..9: note = Description of Artificial Sequence: Synthetic peptide
YLYSTNYMA

SEQ ID NO: 5 - protein, 17 aa, synthetic construct
  source 1..17: note = Description of Artificial Sequence: Synthetic peptide
VIYTASGATLYTDSVKG

SEQ ID NO: 6 - protein, 16 aa, synthetic construct
  source 1..16: note = Description of Artificial Sequence: Synthetic peptide
VRKTDSYLFDAQSFTY

SEQ ID NO: 7 - protein, 9 aa, synthetic construct
  source 1..9: note = Description of Artificial Sequence: Synthetic peptide
YLYSTNYMA

SEQ ID NO: 8 - protein, 17 aa, synthetic construct
  source 1..17: note = Description of Artificial Sequence: Synthetic peptide
VIYTASGATLYTDSVKG

SEQ ID NO: 9 - protein, 16 aa, synthetic construct
  source 1..16: note = Description of Artificial Sequence: Synthetic peptide
VRKTDSYLFDAQSFTY

SEQ ID NO: 10 - protein, 9 aa, synthetic construct
  source 1..9: note = Description of Artificial Sequence: Synthetic peptide
YLYSIDYMA

SEQ ID NO: 11 - protein, 17 aa, synthetic construct
  source 1..17: note = Description of Artificial Sequence: Synthetic peptide
VIYTASGATFYPDSVKG

SEQ ID NO: 12 - protein, 16 aa, synthetic construct
  source 1..16: note = Description of Artificial Sequence: Synthetic peptide
VRKTDSYLFDAQSFTY

SEQ ID NO: 13 - protein, 9 aa, synthetic construct
  source 1..9: note = Description of Artificial Sequence: Synthetic peptide
YLYSTNYMA

SEQ ID NO: 14 - protein, 17 aa, synthetic construct
  source 1..17: note = Description of Artificial Sequence: Synthetic peptide
AIYTASGATLYSDSNKG

SEQ ID NO: 15 - protein, 16 aa, synthetic construct
  source 1..16: note = Description of Artificial Sequence: Synthetic peptide
VRKTGSYLFDAQSFTY

SEQ ID NO: 16 - protein, 9 aa, synthetic construct
  source 1..9: note = Description of Artificial Sequence: Synthetic peptide
FTYSSYCMG

SEQ ID NO: 17 - protein, 16 aa, synthetic construct
  source 1..16: note = Description of Artificial Sequence: Synthetic peptide
SIDSDGSTSYTDSVKG

SEQ ID NO: 18 - protein, 21 aa, synthetic construct
  source 1..21: note = Description of Artificial Sequence: Synthetic peptide
DLMSTVVPGFCGFLLSAGMDY

SEQ ID NO: 19 - protein, 9 aa, synthetic construct
  source 1..9: note = Description of Artificial Sequence: Synthetic peptide
YTFNSNCMG

SEQ ID NO: 20 - protein, 17 aa, synthetic construct
  source 1..17: note = Description of Artificial Sequence: Synthetic peptide
TIYTGVGSTYYADSVKG

SEQ ID NO: 21 - protein, 18 aa, synthetic construct
  source 1..18: note = Description of Artificial Sequence: Synthetic peptide
EPLSRVYGGSCPTPTFGY

SEQ ID NO: 22 - protein, 9 aa, synthetic construct
  source 1..9: note = Description of Artificial Sequence: Synthetic peptide
YTYSMYCMG

SEQ ID NO: 23 - protein, 16 aa, synthetic construct
  source 1..16: note = Description of Artificial Sequence: Synthetic peptide
QINSDGSTSYADSVKG

SEQ ID NO: 24 - protein, 23 aa, synthetic construct
  source 1..23: note = Description of Artificial Sequence: Synthetic peptide
DSRVYGGSWYERLCGPYTYEYNY

SEQ ID NO: 25 - protein, 9 aa, synthetic construct
  source 1..9: note = Description of Artificial Sequence: Synthetic peptide
YAYSTYCMG

SEQ ID NO: 26 - protein, 16 aa, synthetic construct
  source 1..16: note = Description of Artificial Sequence: Synthetic peptide
AIDSGGSTSYADSVKG

SEQ ID NO: 27 - protein, 26 aa, synthetic construct
  source 1..26: note = Description of Artificial Sequence: Synthetic peptide
VPPPPDGGSCLFLGPEIKVSKADFRY

SEQ ID NO: 28 - protein, 9 aa, synthetic construct
  source 1..9: note = Description of Artificial Sequence: Synthetic peptide
YLYSIDYMA

SEQ ID NO: 29 - protein, 17 aa, synthetic construct
  source 1..17: note = Description of Artificial Sequence: Synthetic peptide
VIYTASGATFYPDSVKG

SEQ ID NO: 30 - protein, 16 aa, synthetic construct
  source 1..16: note = Description of Artificial Sequence: Synthetic peptide
VRKTDSYLFDAQSFTY

SEQ ID NO: 31 - protein, 9 aa, synthetic construct
  source 1..9: note = Description of Artificial Sequence: Synthetic peptide
YTYSSYCMG

SEQ ID NO: 32 - protein, 16 aa, synthetic construct
  source 1..16: note = Description of Artificial Sequence: Synthetic peptide
VIDSDGSTSYADSVKG

SEQ ID NO: 33 - protein, 18 aa, synthetic construct
  source 1..18: note = Description of Artificial Sequence: Synthetic peptide
DLGHYRPPCGVLYLGMDY

SEQ ID NO: 34 - protein, 9 aa, synthetic construct
  source 1..9: note = Description of Artificial Sequence: Synthetic peptide
YTYSSNCMG

SEQ ID NO: 35 - protein, 17 aa, synthetic construct
  source 1..17: note = Description of Artificial Sequence: Synthetic peptide
TIYTGGGNTYYADSVKG

SEQ ID NO: 36 - protein, 18 aa, synthetic construct
  source 1..18: note = Description of Artificial Sequence: Synthetic peptide
EPLSRVYGGSCPTPTFDY

SEQ ID NO: 37 - protein, 9 aa, synthetic construct
  source 1..9: note = Description of Artificial Sequence: Synthetic peptide
YSYSSNCMG

SEQ ID NO: 38 - protein, 17 aa, synthetic construct
  source 1..17: note = Description of Artificial Sequence: Synthetic peptide
TIHTGGGSTYYADSVKG

SEQ ID NO: 39 - protein, 18 aa, synthetic construct
  source 1..18: note = Description of Artificial Sequence: Synthetic peptide
EPLSRLYGGSCPTPTFGY

SEQ ID NO: 40 - protein, 9 aa, synthetic construct
  source 1..9: note = Description of Artificial Sequence: Synthetic peptide
YTYSSYCMG

SEQ ID NO: 41 - protein, 16 aa, synthetic construct
  source 1..16: note = Description of Artificial Sequence: Synthetic peptide
VIDSDGSTSYADSVKG

SEQ ID NO: 42 - protein, 18 aa, synthetic construct
  source 1..18: note = Description of Artificial Sequence: Synthetic peptide
DLGHYRPPCGVLYLGMDY

SEQ ID NO: 43 - protein, 9 aa, synthetic construct
  source 1..9: note = Description of Artificial Sequence: Synthetic peptide
YTYSGYCMG

SEQ ID NO: 44 - protein, 16 aa, synthetic construct
  source 1..16: note = Description of Artificial Sequence: Synthetic peptide
VIDSDGSTSYADSVKG

SEQ ID NO: 45 - protein, 18 aa, synthetic construct
  source 1..18: note = Description of Artificial Sequence: Synthetic peptide
DLGHYRPPCGVLYLGMDY

SEQ ID NO: 46 - protein, 9 aa, synthetic construct
  source 1..9: note = Description of Artificial Sequence: Synthetic peptide
YTYSNYCMG

SEQ ID NO: 47 - protein, 16 aa, synthetic construct
  source 1..16: note = Description of Artificial Sequence: Synthetic peptide
TIDSDGNTSYADSVKG

SEQ ID NO: 48 - protein, 18 aa, synthetic construct
  source 1..18: note = Description of Artificial Sequence: Synthetic peptide
DLGHYRPPCGAYYYGMDY

SEQ ID NO: 49 - protein, 9 aa, synthetic construct
  source 1..9: note = Description of Artificial Sequence: Synthetic peptide
YSNCSYDMT

SEQ ID NO: 50 - protein, 16 aa, synthetic construct
  source 1..16: note = Description of Artificial Sequence: Synthetic peptide
AIHSDGSTRYADSVKG

SEQ ID NO: 51 - protein, 19 aa, synthetic construct
  source 1..19: note = Description of Artificial Sequence: Synthetic peptide
DPLHCRAHGGSWYSVRANY

SEQ ID NO: 52 - protein, 9 aa, synthetic construct
  source 1..9: note = Description of Artificial Sequence: Synthetic peptide
YTYNSNCMG

SEQ ID NO: 53 - protein, 17 aa, synthetic construct
  source 1..17: note = Description of Artificial Sequence: Synthetic peptide
TIYTGVGSTYYADSVKG

SEQ ID NO: 54 - protein, 18 aa, synthetic construct
  source 1..18: note = Description of Artificial Sequence: Synthetic peptide
EPLSRVYGGSCPTPTFGY

SEQ ID NO: 55 - protein, 9 aa, synthetic construct
  source 1..9: note = Description of Artificial Sequence: Synthetic peptide
YTYSSGCMG

SEQ ID NO: 56 - protein, 16 aa, synthetic construct
  source 1..16: note = Description of Artificial Sequence: Synthetic peptide
AINSDGSTSYADSVKG

SEQ ID NO: 57 - protein, 18 aa, synthetic construct
  source 1..18: note = Description of Artificial Sequence: Synthetic peptide
EPYCSGGYPRWSVAEFGY

SEQ ID NO: 58 - protein, 9 aa, synthetic construct
  source 1..9: note = Description of Artificial Sequence: Synthetic peptide
YTYSSYCMG

SEQ ID NO: 59 - protein, 16 aa, synthetic construct
  source 1..16: note = Description of Artificial Sequence: Synthetic peptide
AIDSDGSTRYADSVKG

SEQ ID NO: 60 - protein, 18 aa, synthetic construct
  source 1..18: note = Description of Artificial Sequence: Synthetic peptide
EPYCSGGYKRTMVAEFGY

SEQ ID NO: 61 - protein, 9 aa, synthetic construct
  source 1..9: note = Description of Artificial Sequence: Synthetic peptide
YTYNSYCMG

SEQ ID NO: 62 - protein, 16 aa, synthetic construct
  source 1..16: note = Description of Artificial Sequence: Synthetic peptide
TIDSDGMTRYADSVKG

SEQ ID NO: 63 - protein, 13 aa, synthetic construct
  source 1..13: note = Description of Artificial Sequence: Synthetic peptide
DADCTIAAMTTNP

SEQ ID NO: 64 - protein, 9 aa, synthetic construct
  source 1..9: note = Description of Artificial Sequence: Synthetic peptide
YLYSIDYMA

SEQ ID NO: 65 - protein, 17 aa, synthetic construct
  source 1..17: note = Description of Artificial Sequence: Synthetic peptide
VIYTASGATFYPDSVKG

SEQ ID NO: 66 - protein, 16 aa, synthetic construct
  source 1..16: note = Description of Artificial Sequence: Synthetic peptide
VRKTDSYLFDAQSFTY

SEQ ID NO: 67 - protein, 9 aa, synthetic construct
  source 1..9: note = Description of Artificial Sequence: Synthetic peptide
YTYSSYCMG

SEQ ID NO: 68 - protein, 16 aa, synthetic construct
  source 1..16: note = Description of Artificial Sequence: Synthetic peptide
HIDSDGSTTYADSVKG

SEQ ID NO: 69 - protein, 16 aa, synthetic construct
  source 1..16: note = Description of Artificial Sequence: Synthetic peptide
DPIPGPGYCDGGPNKY

SEQ ID NO: 70 - protein, 9 aa, synthetic construct
  source 1..9: note = Description of Artificial Sequence: Synthetic peptide
DLYSTNYVA

SEQ ID NO: 71 - protein, 17 aa, synthetic construct
  source 1..17: note = Description of Artificial Sequence: Synthetic peptide
VIYTASGATLYSDSVKG

SEQ ID NO: 72 - protein, 16 aa, synthetic construct
  source 1..16: note = Description of Artificial Sequence: Synthetic peptide
VRKTGHYLFDAQSFTY

SEQ ID NO: 73 - protein, 9 aa, synthetic construct
  source 1..9: note = Description of Artificial Sequence: Synthetic peptide
YTYSSGCMG

SEQ ID NO: 74 - protein, 16 aa, synthetic construct
  source 1..16: note = Description of Artificial Sequence: Synthetic peptide
TINSDGSTNYADSVKG

SEQ ID NO: 75 - protein, 18 aa, synthetic construct
  source 1..18: note = Description of Artificial Sequence: Synthetic peptide
EPYCSGGYPRWSVAEFGY

SEQ ID NO: 76 - protein, 9 aa, synthetic construct
  source 1..9: note = Description of Artificial Sequence: Synthetic peptide
YSYSSYCMG

SEQ ID NO: 77 - protein, 16 aa, synthetic construct
  source 1..16: note = Description of Artificial Sequence: Synthetic peptide
AIASDGSTSYADSVKG

SEQ ID NO: 78 - protein, 18 aa, synthetic construct
  source 1..18: note = Description of Artificial Sequence: Synthetic peptide
EPWCTGGYSRLTPAEYGY

SEQ ID NO: 79 - protein, 9 aa, synthetic construct
  source 1..9: note = Description of Artificial Sequence: Synthetic peptide
YTYSSGCMG

SEQ ID NO: 80 - protein, 16 aa, synthetic construct
  source 1..16: note = Description of Artificial Sequence: Synthetic peptide
TINSDGSTNYADSVKG

SEQ ID NO: 81 - protein, 18 aa, synthetic construct
  source 1..18: note = Description of Artificial Sequence: Synthetic peptide
EPYCSGGYPRWSVAEFGY

SEQ ID NO: 82 - protein, 9 aa, synthetic construct
  source 1..9: note = Description of Artificial Sequence: Synthetic peptide
YTYSSYCMG

SEQ ID NO: 83 - protein, 16 aa, synthetic construct
  source 1..16: note = Description of Artificial Sequence: Synthetic peptide
HIDSDGSTTYADSVKG

SEQ ID NO: 84 - protein, 16 aa, synthetic construct
  source 1..16: note = Description of Artificial Sequence: Synthetic peptide
DPIPGPGYCDGGPNKY

SEQ ID NO: 85 - protein, 9 aa, synthetic construct
  source 1..9: note = Description of Artificial Sequence: Synthetic peptide
YTYSSYCMG

SEQ ID NO: 86 - protein, 16 aa, synthetic construct
  source 1..16: note = Description of Artificial Sequence: Synthetic peptide
AIDSDGSTRYADSVKG

SEQ ID NO: 87 - protein, 18 aa, synthetic construct
  source 1..18: note = Description of Artificial Sequence: Synthetic peptide
EPYCSGGYKRTMVAEFGY

SEQ ID NO: 88 - protein, 9 aa, synthetic construct
  source 1..9: note = Description of Artificial Sequence: Synthetic peptide
YTYSSYCMG

SEQ ID NO: 89 - protein, 16 aa, synthetic construct
  source 1..16: note = Description of Artificial Sequence: Synthetic peptide
HIDSDGSTSYADSVKG

SEQ ID NO: 90 - protein, 16 aa, synthetic construct
  source 1..16: note = Description of Artificial Sequence: Synthetic peptide
DPIPGPGYCDGGPNKY

SEQ ID NO: 91 - protein, 9 aa, synthetic construct
  source 1..9: note = Description of Artificial Sequence: Synthetic peptide
YTYSSYCMG

SEQ ID NO: 92 - protein, 16 aa, synthetic construct
  source 1..16: note = Description of Artificial Sequence: Synthetic peptide
HIDSDGSTSYADSVKG

SEQ ID NO: 93 - protein, 16 aa, synthetic construct
  source 1..16: note = Description of Artificial Sequence: Synthetic peptide
DPIPGPGYCDGGPNNY

SEQ ID NO: 94 - protein, 9 aa, synthetic construct
  source 1..9: note = Description of Artificial Sequence: Synthetic peptide
YTYSSGCMG

SEQ ID NO: 95 - protein, 16 aa, synthetic construct
  source 1..16: note = Description of Artificial Sequence: Synthetic peptide
TINSDGSTNYADSVKG

SEQ ID NO: 96 - protein, 18 aa, synthetic construct
  source 1..18: note = Description of Artificial Sequence: Synthetic peptide
EPYCSGGYPRWSVAEFGY

SEQ ID NO: 97 - protein, 9 aa, synthetic construct
  source 1..9: note = Description of Artificial Sequence: Synthetic peptide
YTASVNYMG

SEQ ID NO: 98 - protein, 17 aa, synthetic construct
  source 1..17: note = Description of Artificial Sequence: Synthetic peptide
TIFTGAGTTYYANSVKG

SEQ ID NO: 99 - protein, 16 aa, synthetic construct
  source 1..16: note = Description of Artificial Sequence: Synthetic peptide
DFRGGLLYRPAYEYTY

SEQ ID NO: 100 - protein, 9 aa, synthetic construct
  source 1..9: note = Description of Artificial Sequence: Synthetic peptide
YTHSSYCMG

SEQ ID NO: 101 - protein, 16 aa, synthetic construct
  source 1..16: note = Description of Artificial Sequence: Synthetic peptide
AIDVDGSTTYADSVKG

SEQ ID NO: 102 - protein, 18 aa, synthetic construct
  source 1..18: note = Description of Artificial Sequence: Synthetic peptide
EFADCSSNYFLPPGAVRY

SEQ ID NO: 103 - protein, 9 aa, synthetic construct
  source 1..9: note = Description of Artificial Sequence: Synthetic peptide
YTASVNYMG

SEQ ID NO: 104 - protein, 17 aa, synthetic construct
  source 1..17: note = Description of Artificial Sequence: Synthetic peptide
TIFTGAGTTYYANSVKG

SEQ ID NO: 105 - protein, 16 aa, synthetic construct
  source 1..16: note = Description of Artificial Sequence: Synthetic peptide
DFRGGLLYRPAYEYTY

SEQ ID NO: 106 - protein, 9 aa, synthetic construct
  source 1..9: note = Description of Artificial Sequence: Synthetic peptide
DTYSSYCMG

SEQ ID NO: 107 - protein, 16 aa, synthetic construct
  source 1..16: note = Description of Artificial Sequence: Synthetic peptide
FIDSDGSTRYADSVEG

SEQ ID NO: 108 - protein, 18 aa, synthetic construct
  source 1..18: note = Description of Artificial Sequence: Synthetic peptide
EPYCSGGYHRKEMAEFGY

SEQ ID NO: 109 - protein, 9 aa, synthetic construct
  source 1..9: note = Description of Artificial Sequence: Synthetic peptide
YTYSSYCMG

SEQ ID NO: 110 - protein, 16 aa, synthetic construct
  source 1..16: note = Description of Artificial Sequence: Synthetic peptide
HIDSDGSTSYADSVKG

SEQ ID NO: 111 - protein, 16 aa, synthetic construct
  source 1..16: note = Description of Artificial Sequence: Synthetic peptide
DPIPGPGYCDGGPNKY

SEQ ID NO: 112 - protein, 9 aa, synthetic construct
  source 1..9: note = Description of Artificial Sequence: Synthetic peptide
YTYSSYCMG

SEQ ID NO: 113 - protein, 16 aa, synthetic construct
  source 1..16: note = Description of Artificial Sequence: Synthetic peptide
HIDSDGSTTYADSVKG

SEQ ID NO: 114 - protein, 16 aa, synthetic construct
  source 1..16: note = Description of Artificial Sequence: Synthetic peptide
DPIPGPGYCDGGPNKY

SEQ ID NO: 115 - protein, 9 aa, synthetic construct
  source 1..9: note = Description of Artificial Sequence: Synthetic peptide
YTASNNCMG

SEQ ID NO: 116 - protein, 16 aa, synthetic construct
  source 1..16: note = Description of Artificial Sequence: Synthetic peptide
VIFTGAGTSYYDSSVG

SEQ ID NO: 117 - protein, 16 aa, synthetic construct
  source 1..16: note = Description of Artificial Sequence: Synthetic peptide
EDDCTLLLMTPNPDDQ

SEQ ID NO: 118 - protein, 9 aa, synthetic construct
  source 1..9: note = Description of Artificial Sequence: Synthetic peptide
YTDSRYCMG

SEQ ID NO: 119 - protein, 16 aa, synthetic construct
  source 1..16: note = Description of Artificial Sequence: Synthetic peptide
HIDSDGSTSYADSVKG

SEQ ID NO: 120 - protein, 16 aa, synthetic construct
  source 1..16: note = Description of Artificial Sequence: Synthetic peptide
DPIPGPGYCDGGPNKY

SEQ ID NO: 121 - protein, 9 aa, synthetic construct
  source 1..9: note = Description of Artificial Sequence: Synthetic peptide
YTYSSYCMG

SEQ ID NO: 122 - protein, 16 aa, synthetic construct
  source 1..16: note = Description of Artificial Sequence: Synthetic peptide
AIDSDGSTRYADSVKG

SEQ ID NO: 123 - protein, 18 aa, synthetic construct
  source 1..18: note = Description of Artificial Sequence: Synthetic peptide
EPYCSGGYKRTMVAEFGF

SEQ ID NO: 124 - protein, 9 aa, synthetic construct
  source 1..9: note = Description of Artificial Sequence: Synthetic peptide
YTYSSYCMG

SEQ ID NO: 125 - protein, 16 aa, synthetic construct
  source 1..16: note = Description of Artificial Sequence: Synthetic peptide
HIDSDGSTTYADSVKG

SEQ ID NO: 126 - protein, 16 aa, synthetic construct
  source 1..16: note = Description of Artificial Sequence: Synthetic peptide
DPIPGPGYCDGGPNNY

SEQ ID NO: 127 - protein, 9 aa, synthetic construct
  source 1..9: note = Description of Artificial Sequence: Synthetic peptide
YTYSSYCMG

SEQ ID NO: 128 - protein, 16 aa, synthetic construct
  source 1..16: note = Description of Artificial Sequence: Synthetic peptide
HIDSDGSTTYADSVKG

SEQ ID NO: 129 - protein, 16 aa, synthetic construct
  source 1..16: note = Description of Artificial Sequence: Synthetic peptide
DPIPGPGYCDGGPNNY

SEQ ID NO: 130 - protein, 9 aa, synthetic construct
  source 1..9: note = Description of Artificial Sequence: Synthetic peptide
YSYSSYCMG

SEQ ID NO: 131 - protein, 16 aa, synthetic construct
  source 1..16: note = Description of Artificial Sequence: Synthetic peptide
TIDSDGMTRYADSVKG

SEQ ID NO: 132 - protein, 16 aa, synthetic construct
  source 1..16: note = Description of Artificial Sequence: Synthetic peptide
PLYDCDSGAVGRNPPY

SEQ ID NO: 133 - protein, 9 aa, synthetic construct
  source 1..9: note = Description of Artificial Sequence: Synthetic peptide
YTYLRGCMG

SEQ ID NO: 134 - protein, 17 aa, synthetic construct
  source 1..17: note = Description of Artificial Sequence: Synthetic peptide
VMDVVGDRRSYIDSVKG

SEQ ID NO: 135 - protein, 13 aa, synthetic construct
  source 1..13: note = Description of Artificial Sequence: Synthetic peptide
GPNCVGWRSGLDY

SEQ ID NO: 136 - protein, 9 aa, synthetic construct
  source 1..9: note = Description of Artificial Sequence: Synthetic peptide
YTASSICMG

SEQ ID NO: 137 - protein, 17 aa, synthetic construct
  source 1..17: note = Description of Artificial Sequence: Synthetic peptide
VITTAASGTYYADSVNG

SEQ ID NO: 138 - protein, 18 aa, synthetic construct
  source 1..18: note = Description of Artificial Sequence: Synthetic peptide
TRRGGDCLDPLQTPAYNT

SEQ ID NO: 139 - protein, 9 aa, synthetic construct
  source 1..9: note = Description of Artificial Sequence: Synthetic peptide
DTYSRKYIA

SEQ ID NO: 140 - protein, 17 aa, synthetic construct
  source 1..17: note = Description of Artificial Sequence: Synthetic peptide
VMYTPGSATYYTDTVMG

SEQ ID NO: 141 - protein, 14 aa, synthetic construct
  source 1..14: note = Description of Artificial Sequence: Synthetic peptide
KASGSMFNFRDYTY

SEQ ID NO: 142 - protein, 9 aa, synthetic construct
  source 1..9: note = Description of Artificial Sequence: Synthetic peptide
YASCSRAMR

SEQ ID NO: 143 - protein, 16 aa, synthetic construct
  source 1..16: note = Description of Artificial Sequence: Synthetic peptide
YIDGVGSTGYADSVKG

SEQ ID NO: 144 - protein, 13 aa, synthetic construct
  source 1..13: note = Description of Artificial Sequence: Synthetic peptide
GCRADGSNSLDNY

SEQ ID NO: 145 - protein, 9 aa, synthetic construct
  source 1..9: note = Description of Artificial Sequence: Synthetic peptide
YTYNRRFMG

SEQ ID NO: 146 - protein, 17 aa, synthetic construct
  source 1..17: note = Description of Artificial Sequence: Synthetic peptide
IIYTPNSSTFYADSVTG

SEQ ID NO: 147 - protein, 16 aa, synthetic construct
  source 1..16: note = Description of Artificial Sequence: Synthetic peptide
ARIASMTELSVRDMDY

SEQ ID NO: 148 - protein, 9 aa, synthetic construct
  source 1..9: note = Description of Artificial Sequence: Synthetic peptide
YIALNACMA

SEQ ID NO: 149 - protein, 17 aa, synthetic construct
  source 1..17: note = Description of Artificial Sequence: Synthetic peptide
TIVTDGSRTYYADSVKG

SEQ ID NO: 150 - protein, 17 aa, synthetic construct
  source 1..17: note = Description of Artificial Sequence: Synthetic peptide
DRRCPVSRAPYEYELRY

SEQ ID NO: 151 - protein, 9 aa, synthetic construct
  source 1..9: note = Description of Artificial Sequence: Synthetic peptide
YTYNGKCMA

SEQ ID NO: 152 - protein, 17 aa, synthetic construct
  source 1..17: note = Description of Artificial Sequence: Synthetic peptide
GIYTGGSSTYYADSVKG

SEQ ID NO: 153 - protein, 14 aa, synthetic construct
  source 1..14: note = Description of Artificial Sequence: Synthetic peptide
SRSCSDLRRRSIAY

SEQ ID NO: 154 - protein, 125 aa, synthetic construct
  source 1..125: note = Description of Artificial Sequence: Synthetic polypeptide
QVQLQESGGGSIQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSS

SEQ ID NO: 155 - protein, 125 aa, synthetic construct
  source 1..125: note = Description of Artificial Sequence: Synthetic polypeptide
QVQLQESGGGSVQAGGSLRLSCVASRYLYSTNYMAWFRQSPGKEREAVAVIYTASGATLYTDSVKGRFTISQDNAKMTVYLQMNRLKSEDTAMYYCAAVRKTDSYLFDAQSFTYWGQGTQVTVSS

SEQ ID NO: 156 - protein, 125 aa, synthetic construct
  source 1..125: note = Description of Artificial Sequence: Synthetic polypeptide
QVQLQESGGGSIQAGGSLRLSCVASRYLYSTNYMAWFRQSPGKEREAVAVIYTASGATLYTDSVKGRFTISQDNAKMTVYLQMNRLKSEDTAMYYCAAVRKTDSYLFDAQSFTYWGQGTQVTVSS

SEQ ID NO: 157 - protein, 125 aa, synthetic construct
  source 1..125: note = Description of Artificial Sequence: Synthetic polypeptide
QVQLQESGGGSIQAGGSLRLSCAASRYLYSIDYMAWFRQSPGKEREPAAVIYTASGATFYPDSVKGRFTISQDNAKMTVYLQMNSLKSEDTAMYYCAAVRKTDSYLFDAQSFTYWGQGTQVTVSS

SEQ ID NO: 158 - protein, 125 aa, synthetic construct
  source 1..125: note = Description of Artificial Sequence: Synthetic polypeptide
QVQLQESGGGSIQAGGSLRLSCVASKYLYSTNYMAWFRQSPGKEREAVAAIYTASGATLYSDSNKGRFTISQDNAKMTVYLQMNSLKSEDTAMYYCAAVRKTGSYLFDAQSFTYWGQGTQVTVSS

SEQ ID NO: 159 - protein, 129 aa, synthetic construct
  source 1..129: note = Description of Artificial Sequence: Synthetic polypeptide
QVQLQESGGGSVQAGGSLRLSCAASRFTYSSYCMGWFRQAPGKEREGVASIDSDGSTSYTDSVKGRFTISKDNAKNTLYLQMNSLKPEDTAMYYCALDLMSTVVPGFCGFLLSAGMDYWGKGTQVTVSS

SEQ ID NO: 160 - protein, 127 aa, synthetic construct
  source 1..127: note = Description of Artificial Sequence: Synthetic polypeptide
QVQLQESGGGSVQAGGSLRLSCAVSGYTFNSNCMGWFRQAPGKEREGVATIYTGVGSTYYADSVKGRFTISQDNAKNTVYLQMNSLKPEDTAMYYCAAEPLSRVYGGSCPTPTFGYWGQGTQVTVSS

SEQ ID NO: 161 - protein, 131 aa, synthetic construct
  source 1..131: note = Description of Artificial Sequence: Synthetic polypeptide
QVQLQESGGGSVQAGGSLRLSCAASGYTYSMYCMGWFRQAPGKEREGVAQINSDGSTSYADSVKGRFTISKDNAKNTLYLQMNSLKPEDTAMYYCAADSRVYGGSWYERLCGPYTYEYNYWGQGTQVTVSS

SEQ ID NO: 162 - protein, 134 aa, synthetic construct
  source 1..134: note = Description of Artificial Sequence: Synthetic polypeptide
QVQLQESGGGSVQAGGSLRLSCAVSGYAYSTYCMGWFRQAPGKEREGVAAIDSGGSTSYADSVKGRFTISKDNAKNTLYLRMNSLKPEDTAMYYCAAVPPPPDGGSCLFLGPEIKVSKADFRYWGQGTQVTVSS

SEQ ID NO: 163 - protein, 125 aa, synthetic construct
  source 1..125: note = Description of Artificial Sequence: Synthetic polypeptide
QVQLQESGGGSVQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSS

SEQ ID NO: 164 - protein, 126 aa, synthetic construct
  source 1..126: note = Description of Artificial Sequence: Synthetic polypeptide
QVQLQESGGGSVQAGGSLRLSCGASRYTYSSYCMGWFRQAPGKEREGVAVIDSDGSTSYADSVKGRFTISKDNGKNTLYLQMNSLKPEDTAMYYCAADLGHYRPPCGVLYLGMDYWGKGTQVTVSS

SEQ ID NO: 165 - protein, 127 aa, synthetic construct
  source 1..127: note = Description of Artificial Sequence: Synthetic polypeptide
QVQLQESGGGSVQAGGSLRLSCTVSGYTYSSNCMGWFRQAPGKEREGVATIYTGGGNTYYADSVKGRFTISQDNAKNTVYLQMNNLKPEDTAMYYCAAEPLSRVYGGSCPTPTFDYWGQGTQVTVSS

SEQ ID NO: 166 - protein, 127 aa, synthetic construct
  source 1..127: note = Description of Artificial Sequence: Synthetic polypeptide
QVQLQESGGGSVQAGGSLRLSCAVSGYSYSSNCMGWFRQAPGKEREGVATIHTGGGSTYYADSVKGRFTISQDNAKNTVYLQMNSLKPEDTAMYYCAAEPLSRLYGGSCPTPTFGYWGQGTQVTVSS

SEQ ID NO: 167 - protein, 126 aa, synthetic construct
  source 1..126: note = Description of Artificial Sequence: Synthetic polypeptide
QVQLQESGGGSVQAGGSLRLSCGASGYTYSSYCMGWFRQVPGKEREGVAVIDSDGSTSYADSVKGRFTISKDNGKNTLYLQMNSLKPEDTAMYYCAADLGHYRPPCGVLYLGMDYWGKGTQVTVSS

SEQ ID NO: 168 - protein, 126 aa, synthetic construct
  source 1..126: note = Description of Artificial Sequence: Synthetic polypeptide
QVQLQESGGGSVQAGGSLRLSCGASGYTYSGYCMGWFRQAPGKEREGVAVIDSDGSTSYADSVKGRFTISKDNGKNTLYLQMNSLKPEDTAMYYCAADLGHYRPPCGVLYLGMDYWGKGTQVTVSS

SEQ ID NO: 169 - protein, 126 aa, synthetic construct
  source 1..126: note = Description of Artificial Sequence: Synthetic polypeptide
QVQLQESGGGSVQAGGSLRLACAASRYTYSNYCMGWFRQAPGKEREGVATIDSDGNTSYADSVKGRFTISRDNAKNTLYLQMNSLKPGDTAMYYCAADLGHYRPPCGAYYYGMDYWGKGTQVTVSS

SEQ ID NO: 170 - protein, 127 aa, synthetic construct
  source 1..127: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSS

SEQ ID NO: 171 - protein, 127 aa, synthetic construct
  source 1..127: note = Description of Artificial Sequence: Synthetic polypeptide
QVQLQESGGGSVQAGGSLRLSCAVSGYTYNSNCMGWFRQAPGKEREGVATIYTGVGSTYYADSVKGRFTISQDNAKNTVYLQMNSLKPEDTAMYYCAAEPLSRVYGGSCPTPTFGYWGQGTQVTVSS

SEQ ID NO: 172 - protein, 126 aa, synthetic construct
  source 1..126: note = Description of Artificial Sequence: Synthetic polypeptide
QVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSS

SEQ ID NO: 173 - protein, 126 aa, synthetic construct
  source 1..126: note = Description of Artificial Sequence: Synthetic polypeptide
QVQLQESGGGSVQAGGSLRLSCAASGYTYSSYCMGWFRQAPGKEREGVAAIDSDGSTRYADSVKGRFTISKDNAKNTLYLQMNSLKPEDTAMYYCAAEPYCSGGYKRTMVAEFGYWGQGTQVTVSS

SEQ ID NO: 174 - protein, 121 aa, synthetic construct
  source 1..121: note = Description of Artificial Sequence: Synthetic polypeptide
QVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSS

SEQ ID NO: 175 - protein, 125 aa, synthetic construct; identical to SEQ ID NO: 154
  source 1..125: note = Description of Artificial Sequence: Synthetic polypeptide

SEQ ID NO: 176 - protein, 124 aa, synthetic construct
  source 1..124: note = Description of Artificial Sequence: Synthetic polypeptide
QVQLQESGGGLVQPGGSLRLSCAASGYTYSSYCMGWFRQAPGKEREGVAHIDSDGSTTYADSVKGRFTISKDNAKNTLYLQMNSLKPEDTAMYYCAADPIPGPGYCDGGPNKYWGQGTQVTVSS

SEQ ID NO: 177 - protein, 125 aa, synthetic construct
  source 1..125: note = Description of Artificial Sequence: Synthetic polypeptide
QVQLQESGGGSIQAGGSLTLSCAASRDLYSTNYVAWFRQSPGKEREAVAVIYTASGATLYSDSVKGRFTISQDNAKMTVYLQMNSLKSEDTAMYYCAAVRKTGHYLFDAQSFTYWGQGTQVTVSS

SEQ ID NO: 178 - protein, 126 aa, synthetic construct
  source 1..126: note = Description of Artificial Sequence: Synthetic polypeptide
QVQLQESGGGSVQAGGSLRLSCAASGYTYSSGCMGWFRQAPGKEREGVATINSDGSTNYADSVKGRFTISKDNAKNTLYLQMNSLKPEDTAMYYCAAEPYCSGGYPRWSVAEFGYWGQGTQVTVSS

SEQ ID NO: 179 - protein, 126 aa, synthetic construct
  source 1..126: note = Description of Artificial Sequence: Synthetic polypeptide
QVQLQESGGGSVQAGGSLRLSCAASGYSYSSYCMGWFRQAPGKEREGVAAIASDGSTSYADSVKGRFAISKDNAKNTLYLQMASLKPEDTAMYYCAAEPWCTGGYSRLTPAEYGYWGQGTQVTVSS

SEQ ID NO: 180 - protein, 126 aa, synthetic construct
  source 1..126: note = Description of Artificial Sequence: Synthetic polypeptide
QVQLQESGGGLVQPGGSLRLSCAASGYTYSSGCMGWFRQAPGKEREGVATINSDGSTNYADSVKGRFTISKDNAKNTLYLQMNSLKPEDTAMYYCAAEPYCSGGYPRWSVAEFGYWGQGTQVTVSS

SEQ ID NO: 181 - protein, 124 aa, synthetic construct
  source 1..124: note = Description of Artificial Sequence: Synthetic polypeptide
QVQLQESGGGLVQPGGSLRLSCAASGYTYSSYCMGWFRQAPGKEREGVAHIDSDGSTTYADSVKGRFAISKDNAKNTLYLQMNSLKPEDTAMYYCAADPIPGPGYCDGGPNKYWGQGTQVTVSS

SEQ ID NO: 182 - protein, 126 aa, synthetic construct
  source 1..126: note = Description of Artificial Sequence: Synthetic polypeptide
QVQLQESGGGSVQAGGSLRLSCAASGYTYSSYCMGWFRQAPGKGREGVAAIDSDGSTRYADSVKGRFTISKDNAKNTLYLQMNSLKPEDTAMYYCAAEPYCSGGYKRTMVAEFGYWGQGTQVTVSS

SEQ ID NO: 183 - protein, 124 aa, synthetic construct
  source 1..124: note = Description of Artificial Sequence: Synthetic polypeptide
QVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSS

SEQ ID NO: 184 - protein, 124 aa, synthetic construct
  source 1..124: note = Description of Artificial Sequence: Synthetic polypeptide
QVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNNYWGQGTQVTVSS

SEQ ID NO: 185 - protein, 126 aa, synthetic construct
  source 1..126: note = Description of Artificial Sequence: Synthetic polypeptide
QVQLQESGGGSVQAGGSLRLSCAASGYTYSSGCMGWFRQAPGKEREGVATINSDGSTNYADSVKGRFTISKDNAKNTLYLQMNSLKPEDTGMYYCAAEPYCSGGYPRWSVAEFGYWGQGTQVTVSS

SEQ ID NO: 186 - protein, 125 aa, synthetic construct
  source 1..125: note = Description of Artificial Sequence: Synthetic polypeptide
QVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSS

SEQ ID NO: 187 - protein, 126 aa, synthetic construct
  source 1..126: note = Description of Artificial Sequence: Synthetic polypeptide
QVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSS

SEQ ID NO: 188 - protein, 125 aa, synthetic construct
  source 1..125: note = Description of Artificial Sequence: Synthetic polypeptide
QVQLQESGGGSVQAGGSLRLSCTVSRYTASVNYMGWFRQAPGKEREGVATIFTGAGTTYYANSVKGRFTISRDNAKNTAYLQMNSLKPEDTAMYYCAVDFRGGLLYRPAYEYTYRGQGTQVTVSS

SEQ ID NO: 189 - protein, 126 aa, synthetic construct
  source 1..126: note = Description of Artificial Sequence: Synthetic polypeptide
QVQLQESGGGSVQAGGSLRLSCAASGDTYSSYCMGWFRQAPGKEREGVAFIDSDGSTRYADSVEGRFTISKDNAKNTLYLQMNSLKPEDTAMYYCAAEPYCSGGYHRKEMAEFGYWGQGTQVTVSS

SEQ ID NO: 190 - protein, 124 aa, synthetic construct
  source 1..124: note = Description of Artificial Sequence: Synthetic polypeptide
QVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYRGQGTQVTVSS

SEQ ID NO: 191 - protein, 124 aa, synthetic construct
  source 1..124: note = Description of Artificial Sequence: Synthetic polypeptide
QVQLQESGGGSVQAGGSLRLSCAASGYTYSSYCMGWFRQAPGKEREGVAHIDSDGSTTYADSVKGRFAISKDNAKNTLYLQMNSLKPEDTAMYYCAADPIPGPGYCDGGPNKYWGQGTQVTVSS

SEQ ID NO: 192 - protein, 121 aa, synthetic construct
  source 1..121: note = Description of Artificial Sequence: Synthetic polypeptide
QVQLQESGGGSVQAGGSLRLSCTGSGYTASNNCMGWFRQAPGKEREGVAVIFTGAGTSYYDSSVGRLFISSQDAASTLDQLLMSLLPDDTAVMYCGAEDDCTLLLMTPNPDDQWSRLSVSS

SEQ ID NO: 193 - protein, 124 aa, synthetic construct
  source 1..124: note = Description of Artificial Sequence: Synthetic polypeptide
QVQLQESGGGSVQAGGSLRLSCAASGYTDSRYCMGWFRKAPGKEREGVAHIDSDGSTSYADSVKGRFTISKDNAKNTLYLQMNSLKPEDTAMYYCAADPIPGPGYCDGGPNKYWGQGTQVTVSS

SEQ ID NO: 194 - protein, 126 aa, synthetic construct
  source 1..126: note = Description of Artificial Sequence: Synthetic polypeptide
QVQLQESGGGSVQAGGSLRLSCAASGYTYSSYCMGWFRQAPGKEREGVAAIDSDGSTRYADSVKGRFTISKDNAKKILYLQMNSLKVEDTAMYYCAAEPYCSGGYKRTMVAEFGFWGQGTQVTVSS

SEQ ID NO: 195 - protein, 124 aa, synthetic construct
  source 1..124: note = Description of Artificial Sequence: Synthetic polypeptide
QVQLQESGGGSVQAGGSLKLSCAASGYTYSSYCMGWFRQAPGKEREGVAHIDSDGSTTYADSVKGRFTISKDNAKNTLYLQMNSLKPEDTAMYYCAADPIPGPGYCDGGPNNYWGQGTQVTVSS

SEQ ID NO: 196 - protein, 124 aa, synthetic construct
  source 1..124: note = Description of Artificial Sequence: Synthetic polypeptide
QVQLQESGGGSVQAGGSLRLSCAASGYTYSSYCMGWFRQAPGKEREGIAHIDSDGSTTYADSVKGRFTISKDNAKNTLYLQMNSLKPEDTAMYYCAADPIPGPGYCDGGPNNYWGQGTQVTVSS

SEQ ID NO: 197 - protein, 124 aa, synthetic construct
  source 1..124: note = Description of Artificial Sequence: Synthetic polypeptide
QVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSS

SEQ ID NO: 198 - protein, 122 aa, synthetic construct
  source 1..122: note = Description of Artificial Sequence: Synthetic polypeptide
QVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSS

SEQ ID NO: 199 - protein, 127 aa, synthetic construct
  source 1..127: note = Description of Artificial Sequence: Synthetic polypeptide
QVQLQESGGGSVQAGGALRLSCTASGYTASSICMGWFRQAPGKERERVAVITTAASGTYYADSVNGRFSISQNNAKNTVYLQMNSLKPDDTAMYYCAATRRGGDCLDPLQTPAYNTWGQGTQVTVSS

SEQ ID NO: 200 - protein, 123 aa, synthetic construct
  source 1..123: note = Description of Artificial Sequence: Synthetic polypeptide
QVQLQESGGGSVQAGGSLRLSCVASGDTYSRKYIAWVRQVPGKEREGVAVMYTPGSATYYTDTVMGRFTISQDNAKNTVYLQMNSLKPEDTAMYFCAAKASGSMFNFRDYTYWGQGTQVTVSS

SEQ ID NO: 201 - protein, 121 aa, synthetic construct
  source 1..121: note = Description of Artificial Sequence: Synthetic polypeptide
QVQLQESGGGSVQAGGSLRLSCATSGYASCSRAMRWYRQAPGKEREFVAYIDGVGSTGYADSVKGRFTISQDNAKYTAYLQMNSLKPEDTAMYYCNRGCRADGSNSLDNYWGQGTQVTVSS

SEQ ID NO: 202 - protein, 125 aa, synthetic construct
  source 1..125: note = Description of Artificial Sequence: Synthetic polypeptide
QVQLQESGGGSVQAGGSLRLSCAASGYTYNRRFMGWFRQAPGKEREGLAIIYTPNSSTFYADSVTGRFTISQDSARNTVYLQMNSLKPEDTAMYYCAAARIASMTELSVRDMDYWGKGTQVTVSS

SEQ ID NO: 203 - protein, 126 aa, synthetic construct
  source 1..126: note = Description of Artificial Sequence: Synthetic polypeptide
QVQLQESGGGSVQAGGSLRLSCTASRYIALNACMAWIRQAPGSEREVVATIVTDGSRTYYADSVKGRFTISQDNAKNTMYLQMNGLKPEDTAMYYCAADRRCPVSRAPYEYELRYWGQGTQVTVSS

SEQ ID NO: 204 - protein, 123 aa, synthetic construct
  source 1..123: note = Description of Artificial Sequence: Synthetic polypeptide
QVQLQESGGGSVQAGGSLRLSCAASGYTYNGKCMAWFRQAPGKEREVVAGIYTGGSSTYYADSVKGRFTISQDNAKNTVYLQMDSLKPEDTAMYYCATSRSCSDLRRRSIAYWGQGTQVTVSS

SEQ ID NO: 205 - DNA, 375 bp, synthetic construct
  source 1..375: mol_type = other DNA; note = Description of Artificial Sequence: Synthetic polynucleotide
caggttcagcttcaggagtccggtggaggctccatccaggccgggggctctctccgcctgtcttgcgccgcttccagatacctctacagtatcgactacatggcttggtttcgtcagagcccaggaaaagagcgggaacccgtggcagtaatctacactgcctcaggtgccacattttaccccgactctgtcaagggcaggttcaccatctctcaggataatgccaagatgacagtgtacttgcagatgaactccctgaaatctgaggataccgctatgtattactgtgccgcagtgcgcaagaccgattcttacctgttcgacgctcagagttttacctactggggccagggcactcaggtcaccgtcagcagc

SEQ ID NO: 206 - DNA, 375 bp, synthetic construct
  source 1..375: mol_type = other DNA; note = Description of Artificial Sequence: Synthetic polynucleotide
caggtgcagttgcaggagtccggcgggggttccgtgcaagcaggcggatctctgcgcctgtcctgcgtggcctctcgttatttgtatagcaccaactacatggcttggttccgtcagtccccaggcaaagagcgcgaagccgtagccgtaatctatacggcctctggggcaacactctataccgactcagtgaagggacgcttcacgatttcccaagacaatgcaaagatgaccgtgtacttgcagatgaaccgcctgaagagcgaggacacggctatgtattactgcgcagccgtgcgcaagaccgactcctacttgtttgacgctcagtccttcacttattggggccagggtacacaggtcaccgtgagcagt

SEQ ID NO: 207 - DNA, 375 bp, synthetic construct
  source 1..375: mol_type = other DNA; note = Description of Artificial Sequence: Synthetic polynucleotide
caagtacagctccaggagagcggcggtggatctatccaagcagggggtagccttaggttgtcctgtgtggcgtccagatacctgtatagcacgaactacatggcatggttcagacagtccccaggcaaggaacgcgaggcagtcgccgttatttacactgcatctggggccaccctctatacggacagcgtgaagggaaggtttacaatctcccaggacaacgcgaagatgaccgtgtaccttcagatgaaccgcctgaagtccgaggacaccgccatgtattactgtgcagcggtgcgcaagaccgacagctatctgttcgacgcgcagtcattcacttattggggccaaggaacccaagtgaccgtcagctca

SEQ ID NO: 208 - DNA, 375 bp, synthetic construct
  source 1..375: mol_type = other DNA; note = Description of Artificial Sequence: Synthetic polynucleotide
caggtgcagctccaagagtccgggggaggctctatccaggcgggaggcagtctgcgcttgtcctgcgccgcaagtcgttatctgtactccattgattacatggcatggttccgccagtccccaggtaaggaacgtgaacctgccgctgtgatctacaccgcttctggagcaaccttttatcctgatagcgttaagggtcgcttcaccatctctcaggataacgccaaaatgacagtgtacctccagatgaacagcctgaagtctgaggacactgccatgtactattgtgcggctgtgcgcaagaccgactcctatctgtttgatgcacagagctttacctattggggtcagggcacccaggtgactgtgtctagc

SEQ ID NO: 209 - DNA, 375 bp, synthetic construct
  source 1..375: mol_type = other DNA; note = Description of Artificial Sequence: Synthetic polynucleotide
caggtccagttgcaggagtccggtggaggttccatccaggcgggtgggtcccttcgtctctcctgcgtggcctctaagtacctgtattcaaccaactacatggcatggttcagacagtctcccggcaaagagcgtgaggcagtggccgcgatctatacagcttctggggccaccctgtactctgattccaataagggaaggttcactatctcacaggataacgccaaaatgaccgtctaccttcagatgaacagcctcaagtctgaagacacggcaatgtattactgtgcagccgtgcgcaaaactgggagctacctgtttgacgctcagtctttcacttattggggccagggtacgcaggtgacagtctcttct

SEQ ID NO: 210 - DNA, 387 bp, synthetic construct
  source 1..387: mol_type = other DNA; note = Description of Artificial Sequence: Synthetic polynucleotide
caggtgcaactccaggagagcggaggcggttctgttcaggcaggaggttccctgagactgtcctgtgccgcgtctcgctttacgtattcatcctactgcatgggatggttcagacaagcgccggggaaagaaagggaaggcgtggcctccattgactccgacggctcaacttcatacactgatagcgtgaaaggccggttcaccatctctaaggacaacgcgaagaacaccctgtatctccagatgaacagcctcaagcctgaggatactgccatgtactattgcgcactcgacctgatgtctactgtggtcccaggcttctgcgggttcctgctctctgctggcatggactactgggggaagggcactcaggtaacggttagctcc

SEQ ID NO: 211 - DNA, 381 bp, synthetic construct
  source 1..381: mol_type = other DNA; note = Description of Artificial Sequence: Synthetic polynucleotide
caggtgcagcttcaggaatctggcgggggctccgtgcaggccgggggctccctcagactttcctgtgccgtctccggttacacatttaacagtaactgtatgggctggttccgccaggcaccaggcaaggagagggaaggtgtggccacaatctatactggtgttgggagtacgtactatgctgattccgtgaaaggtcgcttcacaatttcccaggacaacgcgaagaacactgtgtacttgcagatgaatagcctgaagcctgaagataccgcaatgtattactgcgctgccgagccactctcccgcgtatatggtggaagttgccccacccccactttcggttactggggccagggcactcaagtgaccgtgtcctct

SEQ ID NO: 212 - DNA, 393 bp, synthetic construct
  source 1..393: mol_type = other DNA; note = Description of Artificial Sequence: Synthetic polynucleotide
caggttcagcttcaggagtctgggggcggttcagtgcaggctggcggttctctccgcctgtcctgcgctgccagcggctatacttacagcatgtactgcatgggctggttccggcaagcccccggcaaagagcgtgagggcgtcgctcaaatcaacagcgacgggtcaaccagctacgccgattctgtcaagggcagatttactatcagcaaggacaacgccaaaaacacactgtacctccagatgaactctttgaagcctgaggacaccgcgatgtattactgcgccgctgacagccgcgtgtacggtggcagctggtatgagaggctgtgcggcccgtacacctacgagtacaactattggggacagggcacgcaggtgacagttagctcc

SEQ ID NO: 213 - DNA, 402 bp, synthetic construct
  source 1..402: mol_type = other DNA; note = Description of Artificial Sequence: Synthetic polynucleotide
caggtgcaactgcaagagagtggcggaggctccgtccaggctggaggttccctgcggctgtcttgcgccgtcagcggctacgcatattccacttactgtatgggttggttccgccaggcccctggaaaggaacgcgagggtgttgccgctattgatagcggaggctccacatcctatgcggactccgtgaaaggtcgtttcaccatctccaaggataacgccaagaacactctgtacctgcgcatgaactctctgaagcctgaggacactgccatgtattactgcgccgctgtgccccctccacccgacgggggctcttgtctgtttcttggcccggagatcaaggtgtccaaggctgatttccgttattggggccagggaactcaagtcaccgtgtcttcc

SEQ ID NO: 214 - DNA, 375 bp, synthetic construct
  source 1..375: mol_type = other DNA; note = Description of Artificial Sequence: Synthetic polynucleotide
caggtccagctccaggagtccggtggaggctccgttcaggccggtggcagcttgcgtctgagctgcgcggcttcaagatacctgtactccattgattacatggcatggttccgtcagtctcctggcaaggagcgcgagcccgtcgctgtgatctataccgccagcggagccacgttctaccctgattccgtcaagggccgcttcaccattagccaagacaacgctaagatgacggtgtacctccaaatgaatagcctgaaaagcgaggacacagcgatgtattactgcgccgctgttaggaaaactgatagttacctgttcgatgcacagtctttcacttactgggggcagggcacccaagttaccgtctcctct

SEQ ID NO: 215 - DNA, 378 bp, synthetic construct
  source 1..378: mol_type = other DNA; note = Description of Artificial Sequence: Synthetic polynucleotide
caggtgcagctccaggaatctggagggggcagtgtgcaggccgggggctccctgcgcttgagctgtggagccagccgctacacgtattccagttactgtatgggctggttcagacaagctccgggtaaggagagagagggagttgccgtaattgattctgacgggtccactagctatgcggattcagtcaagggccggttcaccatcagcaaggacaatggtaagaacacactgtacctgcaaatgaacagcctgaagcccgaggacaccgccatgtactattgtgccgctgatctcggacattaccgccctccctgcggtgtgctctatctcgggatggactattggggtaagggcacccaggtgaccgtgtcctct

SEQ ID NO: 216 - DNA, 381 bp, synthetic construct
  source 1..381: mol_type = other DNA; note = Description of Artificial Sequence: Synthetic polynucleotide
caggtgcagctccaggaaagcggcgggggtagcgttcaagcaggtgggtccctgcgcttgagctgtactgtgtccggctacacctactcaagcaactgcatgggatggttccgtcaggcccctggcaaggaacgcgaaggcgtggctactatctacaccggcggtggcaacacttattacgccgactccgttaaggggcgtttcactatcagccaagacaacgccaagaacaccgtgtatctgcaaatgaataacctgaagcctgaagacaccgccatgtattactgtgctgccgagcccctttcccgcgtttacggcggttcttgtcctacccctacctttgactactggggtcagggaacacaggtgacagtgtccagt

SEQ ID NO: 217 - DNA, 381 bp, synthetic construct
  source 1..381: mol_type = other DNA; note = Description of Artificial Sequence: Synthetic polynucleotide
caagtccaactccaggaatctgggggaggctccgtacaggctggcggttcccttcgtctgtcctgtgctgtgtcagggtactcctactccagtaactgtatgggctggttccggcaagcccccggaaaggagcgcgagggcgtggctaccatccacacagggggcggttccacatattacgccgatagtgtcaagggccgcttcaccattagtcaggacaacgccaagaataccgtttaccttcaaatgaactctttgaaacctgaggacactgcgatgtattactgtgcggcagagcctttgtcccgcctgtacgggggatcttgtccgaccccgactttcgggtactggggacagggcacccaggtgacagtgtcctcc

SEQ ID NO: 218 - DNA, 378 bp, synthetic construct
  source 1..378: mol_type = other DNA; note = Description of Artificial Sequence: Synthetic polynucleotide
caggtgcagttgcaggaaagcgggggtggcagcgtccaagccggtggcagcctgcgtctgtcctgcggtgcctccggctatacttactccagctattgcatgggttggttccgccaagtgccaggaaaggagcgtgagggggtggctgtaattgattcagatgggtcaacaagctacgctgacagcgttaaaggtcgcttcaccatcagtaaggacaacggcaagaacaccctctacctgcaaatgaactccctgaagccggaggataccgcaatgtattactgtgccgctgacttgggacactaccgccctccgtgcggtgtgctttatctgggcatggattactggggtaagggaacccaagtgacggtgtcttct

SEQ ID NO: 219 - DNA, 378 bp, synthetic construct
  source 1..378: mol_type = other DNA; note = Description of Artificial Sequence: Synthetic polynucleotide
caggtacaactccaggagtctggcggtgggtccgtgcaggcaggtggcagccttcgcctctcctgcggggcctccgggtacacctatagtggctactgcatggggtggttcaggcaagcccccggtaaggaacgtgagggagttgctgtgattgattcagatgggtccacgagttacgctgactccgtgaaaggtaggttcacaatctccaaagataatggcaagaacaccctctaccttcagatgaatagcctgaagccagaagacaccgccatgtattactgtgctgccgacctgggacactatcgccctccgtgcggggtcctgtacttgggcatggactattggggcaaggggacccaggtgactgtgtcctct

SEQ ID NO: 220 - DNA, 378 bp, synthetic construct
  source 1..378: mol_type = other DNA; note = Description of Artificial Sequence: Synthetic polynucleotide
caggtgcagttgcaggaatccggtggaggctctgttcaggccgggggctctctccgcctggcctgcgcagcctccaggtatacttacagcaactactgcatggggtggtttcgccaggctccgggcaaagagcgtgagggagtggctactattgattccgatggaaacaccagctacgccgatagcgtgaagggcagatttactatcagcagagataacgctaaaaacacgttgtacctccagatgaactcactcaagccgggggacacagctatgtattactgcgcagccgatctgggtcactaccgcccgccctgcggcgcatattactatggcatggactactggggcaagggcacccaggtgaccgtgtccagt

SEQ ID NO: 221 - DNA, 381 bp, synthetic construct
  source 1..381: mol_type = other DNA; note = Description of Artificial Sequence: Synthetic polynucleotide
caggtgcagctccaagagtctggcgggggttccgtgcaagccggtggctcactcaggttgagttgcgcagccagcggctatagcaactgttcctatgacatgacttggtatcgccaggcccctggcaaagagcgtgagttcgtgtcagctattcactccgacggctccactcgttatgcggactctgtgaagggccggtttttcatctcccaggacaacgctaaaaacactgtctatttgcagatgaactctctgaaacccgaagataccgccatgtactattgcaaaaccgatcctctgcattgtcgcgcccacggcgggagttggtactctgtgcgggccaactattggggccagggcacccaggtcaccgtgtcctca

SEQ ID NO: 222 - DNA, 381 bp, synthetic construct
  source 1..381: mol_type = other DNA; note = Description of Artificial Sequence: Synthetic polynucleotide
caggtacaactccaggagtctggcggtggcagcgtgcaggcaggcggaagcctgaggctgtcctgcgctgtatctggctacacttataattccaactgcatgggttggtttcggcaggctccaggtaaggagcgcgagggcgtcgccaccatttatacaggtgttggcagcacatattacgccgacagcgtgaagggaaggttcaccatctcccaagacaatgcgaaaaacacagtgtatctccagatgaatagcctgaagcccgaggacacggctatgtattactgcgctgccgagccactgagcagagtgtatgggggcagctgtcctacacccactttcggctattggggtcaaggcacccaggttacagtcagctcc

SEQ ID NO: 223 - DNA, 378 bp, synthetic construct
  source 1..378: mol_type = other DNA; note = Description of Artificial Sequence: Synthetic polynucleotide
caggtgcagcttcaggaatcaggcggaggcagcgtgcaggcagggggtagcctgcgtctgtcttgcgcagccagcgggtacacctacagctctggctgtatgggctggtttcgccaagccccaggaaaagaacgggaagccgtggcggctatcaatagcgacggctccacctcctatgctgactccgtcaaaggacgcttcaccattagtaaagataacgccaagaacaccttgtaccttcagatgaactccttgaaaccggaggacaccgcaatgtattactgtgcggctgagccctactgctcaggaggctacccacggtggtcagtggccgagtttggttattggggccagggcacccaagtgactgtgtcctcc

SEQ ID NO: 224 - DNA, 378 bp, synthetic construct
  source 1..378: mol_type = other DNA; note = Description of Artificial Sequence: Synthetic polynucleotide
caggtgcaactccaggagtcagggggaggttccgtgcaggctggcggttctctcaggttgtcttgcgcggccagcggctatacgtacagtagctactgcatgggctggttccggcaagcccccggcaaggagcgcgaaggcgtggctgccattgattccgatggatctactaggtatgctgatagtgtaaagggccgcttcacaatctccaaggacaatgccaagaacacactgtatttgcaaatgaactccctcaagcccgaggataccgctatgtactattgcgctgccgaaccatactgttccggtggctataagcgcactatggtggccgagttcggatactggggtcaaggcacacaggtcacagtgtcctct

SEQ ID NO: 225 - DNA, 363 bp, synthetic construct
  source 1..363: mol_type = other DNA; note = Description of Artificial Sequence: Synthetic polynucleotide
caggtgcagttgcaggagtccgggggcggtagcgttcaggctggagggtccctgcgtctgagttgtgcggcatctcggtatacttataacagttactgtatgggttggttccgccaggcacctggaaaggagcgggagggggtggcgactattgatagcgacggaatgaccagatatgccgactctgtgaagggaagatttactatctcaaaagataatgccaagaacacactctatttgcagatgaacagcctcaagccagaggataccgctatgtattactgtgctgccgacgctgattgcaccatcgctgccatgacgaccaaccccttgggccagggaacccaagtaaccgtctctagc

SEQ ID NO: 226 - DNA, 375 bp, synthetic construct
  source 1..375: mol_type = other DNA; note = Description of Artificial Sequence: Synthetic polynucleotide
caggtccagctccaggaatctggtggcgggtctatccaggcgggtggcagcctgcggctgagttgcgccgcttcccgctacctgtatagtattgattatatggcctggttcaggcagtcaccgggcaaagagcgcgaacccgtcgctgtgatttacacagcctctggtgccaccttctatcccgatagtgtgaagggccggttcactatctctcaagacaacgcgaagatgactgtctatcttcagatgaactctctgaagtccgaggacactgccatgtattactgtgccgctgtgcgcaagacggactcttatctgttcgatgcccagagtttcacttactggggtcagggtactcaggtgaccgtatcctcc

SEQ ID NO: 227 - DNA, 372 bp, synthetic construct
  source 1..372: mol_type = other DNA; note = Description of Artificial Sequence: Synthetic polynucleotide
caggtgcagctccaggagtctggtggcgggctggttcagcctgggggttcactccggttgtcctgcgctgcgtctggttatacctactccagctactgtatgggttggttccgccaggcaccggggaaggagagggagggcgtggctcacattgattctgatggctctacgacctacgctgatagcgttaaggggcgcttcactatctccaaggataacgccaagaacaccctgtatctgcaaatgaacagcctgaagccagaagacactgccatgtactattgcgctgccgatcctattcccggtcctggctattgtgacggcggtcctaacaagtactggggccaaggcacacaggtgactgtcagttcc

SEQ ID NO: 228 - DNA, 375 bp, synthetic construct
  source 1..375: mol_type = other DNA; note = Description of Artificial Sequence: Synthetic polynucleotide
caggttcaactccaggaatccggcggtggaagcattcaggcgggcggttctttgactctgagctgtgcggcatctcgggacctttacagcactaactatgttgcctggttccggcagtcccccggcaaggaacgcgaagctgtggccgtgatttatacagccagcggcgcaaccctgtatagcgattcagtcaaaggccggttcaccatctcccaggacaacgcgaagatgaccgtgtacctgcaaatgaacagcctgaagtctgaggacactgccatgtattactgcgcagctgtgagaaagaccggacattacctcttcgacgcccaatctttcacctactggggccagggaacccaggtcaccgtctcctct

SEQ ID NO: 229 - DNA, 378 bp, synthetic construct
  source 1..378: mol_type = other DNA; note = Description of Artificial Sequence: Synthetic polynucleotide
caggtgcaactccaggagtcaggcggtgggtccgtccaggccggtggctccctgaggctgagttgcgccgcttccggctatacttactccagcggttgcatggggtggttccgccaagcccccggtaaagaacgcgagggagtggctacaattaactccgatggaagcactaactacgccgactctgtgaagggacgcttcaccattagcaaagacaatgctaagaacaccctttaccttcaaatgaacagcctgaagcctgaggataccgctatgtattactgtgccgcagaaccgtattgtagcggtggctaccctcgctggtccgtcgccgagttcggttattggggccaggggacccaagtgactgtttctagc

SEQ ID NO: 230 - DNA, 378 bp, synthetic construct
  source 1..378: mol_type = other DNA; note = Description of Artificial Sequence: Synthetic polynucleotide
caggtgcaacttcaggagagcggcgggggctctgtgcaagctggtggctccctgcggctcagctgtgctgcctctgggtattcttacagtagctactgtatgggctggttcagacaggcaccaggcaaggagcgcgagggtgtggcggccatcgcttccgacgggagtaccagctacgccgacagcgttaaaggtaggtttgccatctctaaggataatgcgaagaatacactctaccttcagatggctagtctgaagccagaggataccgccatgtattactgcgcggcagagccctggtgcacgggagggtattcacgcctgaccccggctgagtatggatactgggggcagggcacccaggtgaccgttagctcc

SEQ ID NO: 231 - DNA, 378 bp, synthetic construct
  source 1..378: mol_type = other DNA; note = Description of Artificial Sequence: Synthetic polynucleotide
caggtccagttgcaggaaagcggagggggcctggtgcagccaggaggttctctgagactgagctgtgccgcttctggttacacatattctagcgggtgcatgggctggttccgccaggctcccggcaaggaacgtgagggtgtggcaactatcaattccgacggctctacaaactacgcagattctgttaaaggccgcttcacaatctctaaggacaacgccaaaaacactctgtacttgcagatgaatagcctgaagcctgaagacactgccatgtactattgcgcagctgagccctactgttctggaggctacccccgctggtctgtggccgagttcggttactggggacaaggaacccaggtcacagtgtccagt

SEQ ID NO: 232 - DNA, 372 bp, synthetic construct
  source 1..372: mol_type = other DNA; note = Description of Artificial Sequence: Synthetic polynucleotide
caggttcagctccaggagtcaggcgggggtcttgtccagcctggtggctccctgcgcctgtcctgtgctgcctccggttacacctactccagctattgcatgggatggttcagacaagcgccaggcaaggaacgtgagggggtcgcccacattgactccgacggttccactacctacgccgacagcgtcaaaggccgcttcgcgatttctaaggataacgctaagaatactctgtacttgcagatgaactcactgaagccagaggacacggccatgtattactgcgcagccgatccgatccccggccccggctattgtgacggtggcccgaacaagtactggggacagggcacccaagtgacggtgtcctct

SEQ ID NO: 233 - DNA, 378 bp, synthetic construct
  source 1..378: mol_type = other DNA; note = Description of Artificial Sequence: Synthetic polynucleotide
caggtacagttgcaggagagcggaggcggttccgtgcaggcaggtggctctcttagactgtcctgcgccgcgagcgggtacacctacagtagctattgtatgggctggttccgccaggctcctggtaagggtcgcgagggcgtcgctgccatcgactccgatggctctactcgctacgcagattctgtcaaggggcgcttcacaatttccaaggacaacgccaagaacacgctttacttgcagatgaactcactgaagccggaggacaccgctatgtattactgcgctgccgagccctactgttctgggggctacaagcgcactatggtggccgagttcggatattggggccagggtacacaggtgaccgtcagttct

SEQ ID NO: 234 - DNA, 372 bp, synthetic construct
  source 1..372: mol_type = other DNA; note = Description of Artificial Sequence: Synthetic polynucleotide
caggtgcagttgcaggagtctggcggtggctctgtgcaggctgggggctctctgcgcctgagttgcgctgccagcggttacacctactccagctattgtatgggatggttccgccaggctccggggaaggagagggagggcgtggcccatatcgactctgatggctccacatcctacgccgacagcgtgaagggacgtttcaccattagcaaggacaatgcgaagaataccctctacttgcagatgaactccctgaagccggaggatactgccatgtattactgcgccgctgatcccatcccagggcctgggtactgtgacggaggcccgaacaagtattggggacaaggaacgcaggtcacagtgtcatct

SEQ ID NO: 235 - DNA, 372 bp, synthetic construct
  source 1..372: mol_type = other DNA; note = Description of Artificial Sequence: Synthetic polynucleotide
caggtacaactccaggagagtggtggaggctccgttcaagccgggggctccctgcggctgtcctgtgcggccagcggttacacctattcatcttactgtatgggctggttccggcaggcccctggtaaggaaagagagggtgtcgctcacattgattccgacggtagtacctcttacgcagactctgtcaagggcaggttcaccatctctaaggacaatgccaagaacaccttgtacctccagatgaactctctgaagcccgaggacactgcaatgtactattgtgcggctgaccctattcccggccctggatattgcgacggcggacctaacaattactggggacagggcacccaggtcaccgtcagctcc

SEQ ID NO: 236 - DNA, 378 bp, synthetic construct
  source 1..378: mol_type = other DNA; note = Description of Artificial Sequence: Synthetic polynucleotide
caggttcagctccaagaatccggcgggggctctgtgcaggcgggcggaagtctgcgtctgtcatgcgctgccagcgggtacacttactcttccggttgtatgggctggtttaggcaggctccgggaaaggaaagggagggcgtcgcaactatcaacagcgacggctctacgaactacgctgactctgtgaaaggccgcttcaccatcagcaaagacaacgccaaaaatacactgtatctccagatgaatagcttgaaacccgaggacaccggaatgtattactgcgcggcagagccatactgttcaggcggttacccaagatggtccgtggctgagttcggttattgggggcagggcactcaggttactgtgtcttcc

SEQ ID NO: 237 - DNA, 375 bp, synthetic construct
  source 1..375: mol_type = other DNA; note = Description of Artificial Sequence: Synthetic polynucleotide
caggtgcagctccaggaatccgggggcggttctgtgcaggctggtggctctctgcgcctgtcttgcactgtttccaggtacactgcctctgtaaactatatgggctggtttagacaagctccgggcaaggaacgcgaaggcgtcgctaccatctttacaggtgcaggtacgacctattacgccaatagcgttaaagggaggttcaccatctccagggacaatgccaaaaacacagcctatctccagatgaactccctcaaacctgaagacacagccatctactattgcgcggttgacttccgtggtggcctgctctatagaccggcgtatgagtacacctaccgtggacaaggcacccaagtcacagtgagcagc

SEQ ID NO: 238 - DNA, 378 bp, synthetic construct
  source 1..378: mol_type = other DNA; note = Description of Artificial Sequence: Synthetic polynucleotide
caggtgcagctccaagagtccggcggagggagtgtagaggctggcgggtccctgcgccttagctgcgcggccagcggctatacacacagttcttattgtatgggttggttccgccaagctccgggaaaggagcgtgagggcgtggctgccatcgacgtggatggctccacaacctacgccgacagcgtgaagggcaggtttacgatctctaaggataacgctaagaatactctctatttgcagatgaactccctcaaacccgaggatacaggaatgtactattgcgctgccgagttcgccgactgctcaagcaattatttcctgcctccaggagccgttaggtactggggccaggggactcaggtcacagtaagcagc

SEQ ID NO: 239 - DNA, 375 bp, synthetic construct
  source 1..375: mol_type = other DNA; note = Description of Artificial Sequence: Synthetic polynucleotide
caggtgcagctccaggagagcggtggcggatcagtgcaggctggaggctccctcagactgtcctgcaccgtgagccgctataccgcctccgtcaactatatgggatggtttaggcaggctccgggcaaggagcgcgagggggtcgcgactatcttcaccggagccggtactacctattacgctaattctgttaaaggccgctttaccattagtcgcgacaacgctaagaacacagcttacctccagatgaactctctgaagccagaggataccgccatgtattactgcgccgtggacttccggggcggtttgctctaccgcccggcctacgaatacacctatcgcggccagggcacgcaggtcacggtgtcctca

SEQ ID NO: 240 - DNA, 378 bp, synthetic construct
  source 1..378: mol_type = other DNA; note = Description of Artificial Sequence: Synthetic polynucleotide
caggtgcagctccaagagtccggtggaggcagcgtccaggccgggggtagtcttaggctcagctgtgctgccagtggagacacctactcttcctattgcatgggatggttcagacaggcccccggcaaagagcgcgagggcgttgcattcatcgactccgacggctccactcgctacgccgatagcgtggagggccgttttaccatctccaaggacaacgcgaagaacactctgtatctgcaaatgaactccctgaagcccgaagacaccgccatgtactattgcgcggctgagccatactgtagtggcggatatcatcgtaaggaaatggcagagttcggctattggggccagggcacccaggtcactgtgagttcc

SEQ ID NO: 241 - DNA, 372 bp, synthetic construct
  source 1..372: mol_type = other DNA; note = Description of Artificial Sequence: Synthetic polynucleotide
caggtgcagttgcaggaatccggcggaggctctgtgcaggcgggcggttccctccgcctgagttgtgccgcgtctggctatacttactcttcctattgtatgggatggttccggcaagcgcccggcaaagagcgggagggcgttgcgcatatcgacagtgatggtagcaccagttacgctgatagcgtgaaaggcagattcactatctcaaaggataacgcgaagaacactctttacctccagatgaactcccttaaacctgaggataccgcgatgtattactgtgctgccgaccccattcccggccctggatactgtgacggaggccctaacaagtaccgtgggcaaggaacacaggtcacagtgtccagc

SEQ ID NO: 242 - DNA, 372 bp, synthetic construct
  source 1..372: mol_type = other DNA; note = Description of Artificial Sequence: Synthetic polynucleotide
caggtgcaactccaggagtctggcgggggcagcgtccaggcaggtggaagtctccgtctctcatgtgctgccagcggctatacatactccagctactgtatgggatggtttagacaggcacccggcaaggagcgcgaaggggtggcccatatcgactccgatggcagcacaacctatgccgactctgtgaaagggcggttcgccatctccaaggacaacgctaagaataccctgtacctccagatgaactctctgaagcctgaggacaccgccatgtattactgcgctgccgacccaatccctggcccaggttactgcgatgggggaccaaacaaatattggggacagggcacgcaggttacagtctccagc

SEQ ID NO: 243 - DNA, 363 bp, synthetic construct
  source 1..363: mol_type = other DNA; note = Description of Artificial Sequence: Synthetic polynucleotide
caggtccaactccaggaaagtggaggtggctctgttcaggccgggggcagcctgaggctgagctgcaccggctcaggctatacagccagtaataactgcatgggctggttccgtcaagcgcccggcaaagagcgtgaaggtgtggccgtaatttttaccggcgctggcaccagctattacgacagttccgtgggccgtctgttcatcagctcacaggacgccgcttccaccctcgatcagttgctgatgagccttctgcccgatgacaccgcagtaatgtactgtggagccgaagatgactgcacactgctcctgatgacgccaaaccccgatgaccaatggtcccgcctgagtgtgtcctcc

SEQ ID NO: 244 - DNA, 372 bp, synthetic construct
  source 1..372: mol_type = other DNA; note = Description of Artificial Sequence: Synthetic polynucleotide
caggtgcagctccaggagagcgggggcggttctgttcaggcgggaggcagcctgcgtctgtcctgtgcagcctctggttacacagacagtcgttactgcatgggctggttccgcaaggcacctggaaaggagcgcgagggtgttgcgcacatcgactccgacgggagcactagctatgctgacagcgtgaaggggcgcttcactatcagcaaggataacgcgaaaaacaccttgtaccttcagatgaactccctcaaacccgaagacacagcgatgtactattgtgccgctgatccgatcccagggcctggctactgtgatggtggacctaataagtactgggggcagggaactcaggtgaccgtgtcatca

SEQ ID NO: 245 - DNA, 378 bp, synthetic construct
  source 1..378: mol_type = other DNA; note = Description of Artificial Sequence: Synthetic polynucleotide
caggtccagttgcaggaatctggaggcggttccgtgcaagcagggggctcactcagactgtcctgcgctgccagcggctacacttactcttcatattgcatgggctggttccgccaggcaccgggcaaggagcgggaaggcgtggccgctattgatagcgatggctctacgcgctacgcagatagcgtgaaagggaggttcacgatctccaaagataatgccaagaaaattctgtatctccagatgaactctctgaaggtcgaggacaccgccatgtactattgtgcagccgaaccctactgttctggtggctacaagaggactatggtggccgagttcggcttctggggccaggggacccaagtgactgtcagtagc

SEQ ID NO: 246 - DNA, 372 bp, synthetic construct
  source 1..372: mol_type = other DNA; note = Description of Artificial Sequence: Synthetic polynucleotide
caggtgcaacttcaggagagcggtggcggatctgtgcaggctggagggtctctgaagctgtcctgcgcggccagcggttacacatacagtagctactgcatgggatggtttcgtcaggccccaggcaaggagcgcgaaggagtggcgcacatcgactccgatgggtccaccacatacgccgactccgtgaagggccgtttcacaatcagcaaggataacgcgaagaacacgctgtacttgcagatgaactctctcaaaccagaggacactgcaatgtactattgcgcggctgaccccatccctggccctggttactgtgacggtggccccaacaattactgggggcaagggacccaagtcaccgtgtcctcc

SEQ ID NO: 247 - DNA, 372 bp, synthetic construct
  source 1..372: mol_type = other DNA; note = Description of Artificial Sequence: Synthetic polynucleotide
caggtccagctccaggagtccggcgggggctccgtccaggcagggggctccctgcgtctgtcatgcgccgcttctgggtatacctacagttcctattgtatgggttggtttcgccaagcacccggtaaggagcgcgaaggtattgcgcacattgatagcgatggctccacaacctatgctgacagtgtgaaaggacgcttcactatttccaaggataacgctaagaacacactctaccttcagatgaacagcctgaagccggaagacaccgcaatgtactattgtgcagctgaccccattcctggacccggttactgtgatggaggtcctaataactattggggacagggcactcaagtgaccgtctcaagc

SEQ ID NO: 248 - DNA, 372 bp, synthetic construct
  source 1..372: mol_type = other DNA; note = Description of Artificial Sequence: Synthetic polynucleotide
caggtgcagttgcaggagagcgggggtggctctgtgcaggccgggggctccctgaggctgagctgcgcggccagcgggtacagctactctagctattgcatgggttggttccgccaggcccctggcaaggagcgcgagggagtggccacgattgactcagatggcatgacccgttatgcggattccgtcaaggggcgcttcaccatcagcaaagataacgccaaaaataccctgtacttgcagatgaactcactgaaacctgaggatacagccatgtattactgcgcagctccgctctatgactgtgactctggtgccgtgggtagaaacccaccttactgggggcagggaacccaggtgaccgtgtcctca

SEQ ID NO: 249 - DNA, 366 bp, synthetic construct
  source 1..366: mol_type = other DNA; note = Description of Artificial Sequence: Synthetic polynucleotide
caggtccagctccaggaaagcggtgggggcagcgtccaaacagggggtagcctgcgcctctcttgcgcagccagcggctacacatatctgcgcggatgtatgggctggttccgccaggcccctggtaaggaaagagagggggtggccgtgatggacgtggttggagacagacgttcctacattgattccgtgaagggccgctttactatctcacgcgataacgcggctaactctgtgtatttgcagatggataacctgaagcccgaggacaccgctatgtactattgcacagctggtcccaactgtgtcggttggcgctccggcctggactattggggtcagggaacccaggttacagttagcagt

SEQ ID NO: 250 - DNA, 381 bp, synthetic construct
  source 1..381: mol_type = other DNA; note = Description of Artificial Sequence: Synthetic polynucleotide
caggtgcagctccaggagagtggtggcggttctgtccaagctggcggagccctgcgcctgtcctgcacagcaagcggctacaccgcctctagcatttgcatgggatggttccgtcaggccccaggcaaggagagggagagagtggctgtgattaccacggcagcctccggtacttactatgccgactctgtgaatggccgcttctcaatctctcagaataacgccaaaaatactgtgtacctccagatgaactccctgaaacctgacgataccgcgatgtattactgcgcagccacccggcgcggcggtgactgcctggacccattgcagaccccagcctataatacctggggccagggaacccaggtcaccgtctcttct

SEQ ID NO: 251 - DNA, 369 bp, synthetic construct
  source 1..369: mol_type = other DNA; note = Description of Artificial Sequence: Synthetic polynucleotide
caggtgcagctccaggaaagcggcggtggctccgtccaggccggtggctccctgaggctgagctgtgtggcttccggcgatacttattctcgcaagtacatcgcatgggtgcgtcaggtgcccggtaaagaacgtgagggagtggcagtgatgtataccccaggctccgctacttactatacagacacagtgatgggtcgtttcaccatctcccaggacaacgccaagaacactgtgtaccttcaaatgaacagcctcaaacctgaagacaccgccatgtacttttgcgcggccaaggccagcggctccatgtttaacttccgcgattacacttattggggacagggcactcaggtgaccgtaagctct

SEQ ID NO: 252 - DNA, 363 bp, synthetic construct
  source 1..363: mol_type = other DNA; note = Description of Artificial Sequence: Synthetic polynucleotide
caggtgcagctgcaagaaagcggaggtggctctgtccaggcaggaggctccctccggcttagctgcgctaccagcgggtatgcttcctgttcccgcgccatgaggtggtacaggcaggcaccgggcaaggagcgcgaatttgtggcgtacatcgacggggtgggcagtactggttatgcggacagcgttaaaggccggtttaccatctcccaagataatgcaaagtacacggcttacttgcagatgaactccctcaagcctgaggataccgcgatgtattactgtaatcggggctgtagagccgatggtagcaatagtctggacaactactggggccagggcacacaggtgactgtctcttca

SEQ ID NO: 253 - DNA, 375 bp, synthetic construct
  source 1..375: mol_type = other DNA; note = Description of Artificial Sequence: Synthetic polynucleotide
caggtgcagttgcaggagtccggcggtggcagcgttcaggcgggcggtagcctgcgtctgagctgcgccgcgtccggctacacctataaccgtcgcttcatgggttggttccgtcaagcgcccggcaaggagagagagggcctcgccattatctacacccccaacagctccaccttctacgccgactctgtgacgggccgctttacaatctcacaggattctgcccgcaacaccgtctatttgcagatgaactccctgaaacctgaggacaccgctatgtactattgtgcagccgctcgcatcgcttctatgactgagctttcagtgagagatatggactattggggcaagggcacccaggtgaccgtttcctcc

SEQ ID NO: 254 - DNA, 378 bp, synthetic construct
  source 1..378: mol_type = other DNA; note = Description of Artificial Sequence: Synthetic polynucleotide
caggtacaactccaggagagcgggggaggtagcgtacaggctggcgggtccttgcgtctgagctgcactgcatctcgttacatcgctcttaatgcgtgtatggcttggattcggcaggcccccggctccgaaagggaggtcgtggccacaatcgtgactgatggctccagaacctattacgcagactctgtcaagggccggtttactatctctcaagacaacgccaagaacaccatgtacctccagatgaacggtttgaaacccgaagacaccgccatgtattactgtgcagccgacaggcgctgccccgtgtccagagccccatacgaatacgaactgcgctactggggtcagggcacccaggtgactgtcagcagc

SEQ ID NO: 255 - DNA, 369 bp, synthetic construct
  source 1..369: mol_type = other DNA; note = Description of Artificial Sequence: Synthetic polynucleotide
caagtccagcttcaagaaagcggagggggctctgttcaggcaggcgggtccctccggctgtcctgcgctgcctccggctacacatacaacggaaagtgcatggcttggttccgccaggctcccggcaaggagcgcgaagtcgtggctggcatttacaccgggggctccagcacatattacgccgatagtgtgaagggacgctttacgatttcccaagacaatgctaaaaatacagtctatctccagatggacagcctgaagcccgaagacactgccatgtattactgcgccaccagcagaagctgtagcgacctgcgcagacgctccatcgcctactggggacaggggactcaggtcaccgtcagctct

SEQ ID NO: 256 - protein, 263 aa, synthetic construct
  source 1..263: note = Description of Artificial Sequence: Synthetic polypeptide
QVQLQESGGGSVQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSSGGGSQVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSSASHHHHHH

SEQ ID NO: 257 - protein, 261 aa, synthetic construct
  source 1..261: note = Description of Artificial Sequence: Synthetic polypeptide
QVQLQESGGGSVQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSSGGGSQVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSSASHHHHHH

SEQ ID NO: 258 - protein, 258 aa, synthetic construct
  source 1..258: note = Description of Artificial Sequence: Synthetic polypeptide
QVQLQESGGGSVQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSSGGGSQVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSASHHHHHH

SEQ ID NO: 259 - protein, 262 aa, synthetic construct
  source 1..262: note = Description of Artificial Sequence: Synthetic polypeptide
QVQLQESGGGSVQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSSGGGSQVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSSASHHHHHH

SEQ ID NO: 260 - protein, 263 aa, synthetic construct
  source 1..263: note = Description of Artificial Sequence: Synthetic polypeptide
QVQLQESGGGSVQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSSGGGSQVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSASHHHHHH

SEQ ID NO: 261 - protein, 261 aa, synthetic construct
  source 1..261: note = Description of Artificial Sequence: Synthetic polypeptide
QVQLQESGGGSVQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSSGGGSQVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSASHHHHHH

SEQ ID NO: 262 - protein, 259 aa, synthetic construct
  source 1..259: note = Description of Artificial Sequence: Synthetic polypeptide
QVQLQESGGGSVQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSSGGGSQVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSSASHHHHHH

SEQ ID NO: 263 - protein, 267 aa, synthetic construct
  source 1..267: note = Description of Artificial Sequence: Synthetic polypeptide
QVQLQESGGGSVQAGGSLRLSCAASRFTYSSYCMGWFRQAPGKEREGVASIDSDGSTSYTDSVKGRFTISKDNAKNTLYLQMNSLKPEDTAMYYCALDLMSTVVPGFCGFLLSAGMDYWGKGTQVTVSSGGGSQVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSSASHHHHHH

SEQ ID NO: 264 - protein, 265 aa, synthetic construct
  source 1..265: note = Description of Artificial Sequence: Synthetic polypeptide
QVQLQESGGGSVQAGGSLRLSCAASRFTYSSYCMGWFRQAPGKEREGVASIDSDGSTSYTDSVKGRFTISKDNAKNTLYLQMNSLKPEDTAMYYCALDLMSTVVPGFCGFLLSAGMDYWGKGTQVTVSSGGGSQVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSSASHHHHHH

SEQ ID NO: 265 - protein, 262 aa, synthetic construct
  source 1..262: note = Description of Artificial Sequence: Synthetic polypeptide
QVQLQESGGGSVQAGGSLRLSCAASRFTYSSYCMGWFRQAPGKEREGVASIDSDGSTSYTDSVKGRFTISKDNAKNTLYLQMNSLKPEDTAMYYCALDLMSTVVPGFCGFLLSAGMDYWGKGTQVTVSSGGGSQVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSASHHHHHH

SEQ ID NO: 266 - protein, 266 aa, synthetic construct
  source 1..266: note = Description of Artificial Sequence: Synthetic polypeptide
QVQLQESGGGSVQAGGSLRLSCAASRFTYSSYCMGWFRQAPGKEREGVASIDSDGSTSYTDSVKGRFTISKDNAKNTLYLQMNSLKPEDTAMYYCALDLMSTVVPGFCGFLLSAGMDYWGKGTQVTVSSGGGSQVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSSASHHHHHH

SEQ ID NO: 267 - protein, 267 aa, synthetic construct
  source 1..267: note = Description of Artificial Sequence: Synthetic polypeptide
QVQLQESGGGSVQAGGSLRLSCAASRFTYSSYCMGWFRQAPGKEREGVASIDSDGSTSYTDSVKGRFTISKDNAKNTLYLQMNSLKPEDTAMYYCALDLMSTVVPGFCGFLLSAGMDYWGKGTQVTVSSGGGSQVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSASHHHHHH

SEQ ID NO: 268 - protein, 265 aa, synthetic construct
  source 1..265: note = Description of Artificial Sequence: Synthetic polypeptide
QVQLQESGGGSVQAGGSLRLSCAASRFTYSSYCMGWFRQAPGKEREGVASIDSDGSTSYTDSVKGRFTISKDNAKNTLYLQMNSLKPEDTAMYYCALDLMSTVVPGFCGFLLSAGMDYWGKGTQVTVSSGGGSQVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSASHHHHHH

SEQ ID NO: 269 - protein, 263 aa, synthetic construct
  source 1..263: note = Description of Artificial Sequence: Synthetic polypeptide
QVQLQESGGGSVQAGGSLRLSCAASRFTYSSYCMGWFRQAPGKEREGVASIDSDGSTSYTDSVKGRFTISKDNAKNTLYLQMNSLKPEDTAMYYCALDLMSTVVPGFCGFLLSAGMDYWGKGTQVTVSSGGGSQVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSSASHHHHHH

SEQ ID NO: 270 - protein, 269 aa, synthetic construct
  source 1..269: note = Description of Artificial Sequence: Synthetic polypeptide
QVQLQESGGGSVQAGGSLRLSCAASGYTYSMYCMGWFRQAPGKEREGVAQINSDGSTSYADSVKGRFTISKDNAKNTLYLQMNSLKPEDTAMYYCAADSRVYGGSWYERLCGPYTYEYNYWGQGTQVTVSSGGGSQVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSSASHHHHHH

SEQ ID NO: 271 - protein, 267 aa, synthetic construct
  source 1..267: note = Description of Artificial Sequence: Synthetic polypeptide
QVQLQESGGGSVQAGGSLRLSCAASGYTYSMYCMGWFRQAPGKEREGVAQINSDGSTSYADSVKGRFTISKDNAKNTLYLQMNSLKPEDTAMYYCAADSRVYGGSWYERLCGPYTYEYNYWGQGTQVTVSSGGGSQVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSSASHHHHHH

SEQ ID NO: 272 - protein, 264 aa, synthetic construct
  source 1..264: note = Description of Artificial Sequence: Synthetic polypeptide
QVQLQESGGGSVQAGGSLRLSCAASGYTYSMYCMGWFRQAPGKEREGVAQINSDGSTSYADSVKGRFTISKDNAKNTLYLQMNSLKPEDTAMYYCAADSRVYGGSWYERLCGPYTYEYNYWGQGTQVTVSSGGGSQVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSASHHHHHH

SEQ ID NO: 273 - protein, 268 aa, synthetic construct
  source 1..268: note = Description of Artificial Sequence: Synthetic polypeptide
QVQLQESGGGSVQAGGSLRLSCAASGYTYSMYCMGWFRQAPGKEREGVAQINSDGSTSYADSVKGRFTISKDNAKNTLYLQMNSLKPEDTAMYYCAADSRVYGGSWYERLCGPYTYEYNYWGQGTQVTVSSGGGSQVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSSASHHHHHH

SEQ ID NO: 274 - protein, 269 aa, synthetic construct
  source 1..269: note = Description of Artificial Sequence: Synthetic polypeptide
QVQLQESGGGSVQAGGSLRLSCAASGYTYSMYCMGWFRQAPGKEREGVAQINSDGSTSYADSVKGRFTISKDNAKNTLYLQMNSLKPEDTAMYYCAADSRVYGGSWYERLCGPYTYEYNYWGQGTQVTVSSGGGSQVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSASHHHHHH

SEQ ID NO: 275 - protein, 267 aa, synthetic construct
  source 1..267: note = Description of Artificial Sequence: Synthetic polypeptide
QVQLQESGGGSVQAGGSLRLSCAASGYTYSMYCMGWFRQAPGKEREGVAQINSDGSTSYADSVKGRFTISKDNAKNTLYLQMNSLKPEDTAMYYCAADSRVYGGSWYERLCGPYTYEYNYWGQGTQVTVSSGGGSQVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSASHHHHHH

SEQ ID NO: 276 - protein, 265 aa, synthetic construct
  source 1..265: note = Description of Artificial Sequence: Synthetic polypeptide
QVQLQESGGGSVQAGGSLRLSCAASGYTYSMYCMGWFRQAPGKEREGVAQINSDGSTSYADSVKGRFTISKDNAKNTLYLQMNSLKPEDTAMYYCAADSRVYGGSWYERLCGPYTYEYNYWGQGTQVTVSSGGGSQVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSSASHHHHHH

SEQ ID NO: 277 - protein, 272 aa, synthetic construct
  source 1..272: note = Description of Artificial Sequence: Synthetic polypeptide
QVQLQESGGGSVQAGGSLRLSCAVSGYAYSTYCMGWFRQAPGKEREGVAAIDSGGSTSYADSVKGRFTISKDNAKNTLYLRMNSLKPEDTAMYYCAAVPPPPDGGSCLFLGPEIKVSKADFRYWGQGTQVTVSSGGGSQVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSSASHHHHHH

SEQ ID NO: 278 - protein, 270 aa, synthetic construct
  source 1..270: note = Description of Artificial Sequence: Synthetic polypeptide
QVQLQESGGGSVQAGGSLRLSCAVSGYAYSTYCMGWFRQAPGKEREGVAAIDSGGSTSYADSVKGRFTISKDNAKNTLYLRMNSLKPEDTAMYYCAAVPPPPDGGSCLFLGPEIKVSKADFRYWGQGTQVTVSSGGGSQVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSSASHHHHHH

SEQ ID NO: 279 - protein, 267 aa, synthetic construct
  source 1..267: note = Description of Artificial Sequence: Synthetic polypeptide
QVQLQESGGGSVQAGGSLRLSCAVSGYAYSTYCMGWFRQAPGKEREGVAAIDSGGSTSYADSVKGRFTISKDNAKNTLYLRMNSLKPEDTAMYYCAAVPPPPDGGSCLFLGPEIKVSKADFRYWGQGTQVTVSSGGGSQVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSASHHHHHH

SEQ ID NO: 280 - protein, 271 aa, synthetic construct
  source 1..271: note = Description of Artificial Sequence: Synthetic polypeptide
QVQLQESGGGSVQAGGSLRLSCAVSGYAYSTYCMGWFRQAPGKEREGVAAIDSGGSTSYADSVKGRFTISKDNAKNTLYLRMNSLKPEDTAMYYCAAVPPPPDGGSCLFLGPEIKVSKADFRYWGQGTQVTVSSGGGSQVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSSASHHHHHH

SEQ ID NO: 281 - protein, 272 aa, synthetic construct
  source 1..272: note = Description of Artificial Sequence: Synthetic polypeptide
QVQLQESGGGSVQAGGSLRLSCAVSGYAYSTYCMGWFRQAPGKEREGVAAIDSGGSTSYADSVKGRFTISKDNAKNTLYLRMNSLKPEDTAMYYCAAVPPPPDGGSCLFLGPEIKVSKADFRYWGQGTQVTVSSGGGSQVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSASHHHHHH

SEQ ID NO: 282 - protein, 270 aa, synthetic construct
  source 1..270: note = Description of Artificial Sequence: Synthetic polypeptide
QVQLQESGGGSVQAGGSLRLSCAVSGYAYSTYCMGWFRQAPGKEREGVAAIDSGGSTSYADSVKGRFTISKDNAKNTLYLRMNSLKPEDTAMYYCAAVPPPPDGGSCLFLGPEIKVSKADFRYWGQGTQVTVSSGGGSQVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSASHHHHHH

SEQ ID NO: 283 - protein, 268 aa, synthetic construct
  source 1..268: note = Description of Artificial Sequence: Synthetic polypeptide
QVQLQESGGGSVQAGGSLRLSCAVSGYAYSTYCMGWFRQAPGKEREGVAAIDSGGSTSYADSVKGRFTISKDNAKNTLYLRMNSLKPEDTAMYYCAAVPPPPDGGSCLFLGPEIKVSKADFRYWGQGTQVTVSSGGGSQVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSSASHHHHHH

SEQ ID NO: 284 - protein, 265 aa, synthetic construct
  source 1..265: note = Description of Artificial Sequence: Synthetic polypeptide
QVQLQESGGGSVQAGGSLRLSCTVSGYTYSSNCMGWFRQAPGKEREGVATIYTGGGNTYYADSVKGRFTISQDNAKNTVYLQMNNLKPEDTAMYYCAAEPLSRVYGGSCPTPTFDYWGQGTQVTVSSGGGSQVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSSASHHHHHH

SEQ ID NO: 285 - protein, 263 aa, synthetic construct
  source 1..263: note = Description of Artificial Sequence: Synthetic polypeptide
QVQLQESGGGSVQAGGSLRLSCTVSGYTYSSNCMGWFRQAPGKEREGVATIYTGGGNTYYADSVKGRFTISQDNAKNTVYLQMNNLKPEDTAMYYCAAEPLSRVYGGSCPTPTFDYWGQGTQVTVSSGGGSQVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSSASHHHHHH

SEQ ID NO: 286 - protein, 260 aa, synthetic construct
  source 1..260: note = Description of Artificial Sequence: Synthetic polypeptide
QVQLQESGGGSVQAGGSLRLSCTVSGYTYSSNCMGWFRQAPGKEREGVATIYTGGGNTYYADSVKGRFTISQDNAKNTVYLQMNNLKPEDTAMYYCAAEPLSRVYGGSCPTPTFDYWGQGTQVTVSSGGGSQVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSASHHHHHH

SEQ ID NO: 287 - protein, 264 aa, synthetic construct
  source 1..264: note = Description of Artificial Sequence: Synthetic polypeptide
QVQLQESGGGSVQAGGSLRLSCTVSGYTYSSNCMGWFRQAPGKEREGVATIYTGGGNTYYADSVKGRFTISQDNAKNTVYLQMNNLKPEDTAMYYCAAEPLSRVYGGSCPTPTFDYWGQGTQVTVSSGGGSQVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSSASHHHHHH

SEQ ID NO: 288 - protein, 265 aa, synthetic construct
  source 1..265: note = Description of Artificial Sequence: Synthetic polypeptide
QVQLQESGGGSVQAGGSLRLSCTVSGYTYSSNCMGWFRQAPGKEREGVATIYTGGGNTYYADSVKGRFTISQDNAKNTVYLQMNNLKPEDTAMYYCAAEPLSRVYGGSCPTPTFDYWGQGTQVTVSSGGGSQVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSASHHHHHH

SEQ ID NO: 289 - protein, 263 aa, synthetic construct
  source 1..263: note = Description of Artificial Sequence: Synthetic polypeptide
QVQLQESGGGSVQAGGSLRLSCTVSGYTYSSNCMGWFRQAPGKEREGVATIYTGGGNTYYADSVKGRFTISQDNAKNTVYLQMNNLKPEDTAMYYCAAEPLSRVYGGSCPTPTFDYWGQGTQVTVSSGGGSQVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSASHHHHHH

SEQ ID NO: 290 - protein, 261 aa, synthetic construct
  source 1..261: note = Description of Artificial Sequence: Synthetic polypeptide
QVQLQESGGGSVQAGGSLRLSCTVSGYTYSSNCMGWFRQAPGKEREGVATIYTGGGNTYYADSVKGRFTISQDNAKNTVYLQMNNLKPEDTAMYYCAAEPLSRVYGGSCPTPTFDYWGQGTQVTVSSGGGSQVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSSASHHHHHH

SEQ ID NO: 291 - protein, 264 aa, synthetic construct
  source 1..264: note = Description of Artificial Sequence: Synthetic polypeptide
QVQLQESGGGSVQAGGSLRLSCGASGYTYSSYCMGWFRQVPGKEREGVAVIDSDGSTSYADSVKGRFTISKDNGKNTLYLQMNSLKPEDTAMYYCAADLGHYRPPCGVLYLGMDYWGKGTQVTVSSGGGSQVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSSASHHHHHH

SEQ ID NO: 292 - protein, 262 aa, synthetic construct
  source 1..262: note = Description of Artificial Sequence: Synthetic polypeptide
QVQLQESGGGSVQAGGSLRLSCGASGYTYSSYCMGWFRQVPGKEREGVAVIDSDGSTSYADSVKGRFTISKDNGKNTLYLQMNSLKPEDTAMYYCAADLGHYRPPCGVLYLGMDYWGKGTQVTVSSGGGSQVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSSASHHHHHH

SEQ ID NO: 293 - protein, 259 aa, synthetic construct
  source 1..259: note = Description of Artificial Sequence: Synthetic polypeptide
QVQLQESGGGSVQAGGSLRLSCGASGYTYSSYCMGWFRQVPGKEREGVAVIDSDGSTSYADSVKGRFTISKDNGKNTLYLQMNSLKPEDTAMYYCAADLGHYRPPCGVLYLGMDYWGKGTQVTVSSGGGSQVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSASHHHHHH

SEQ ID NO: 294 - protein, 263 aa, synthetic construct
  source 1..263: note = Description of Artificial Sequence: Synthetic polypeptide
QVQLQESGGGSVQAGGSLRLSCGASGYTYSSYCMGWFRQVPGKEREGVAVIDSDGSTSYADSVKGRFTISKDNGKNTLYLQMNSLKPEDTAMYYCAADLGHYRPPCGVLYLGMDYWGKGTQVTVSSGGGSQVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSSASHHHHHH

SEQ ID NO: 295 - protein, 264 aa, synthetic construct
  source 1..264: note = Description of Artificial Sequence: Synthetic polypeptide
QVQLQESGGGSVQAGGSLRLSCGASGYTYSSYCMGWFRQVPGKEREGVAVIDSDGSTSYADSVKGRFTISKDNGKNTLYLQMNSLKPEDTAMYYCAADLGHYRPPCGVLYLGMDYWGKGTQVTVSSGGGSQVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSASHHHHHH

SEQ ID NO: 296 - protein, 262 aa, synthetic construct
  source 1..262: note = Description of Artificial Sequence: Synthetic polypeptide
QVQLQESGGGSVQAGGSLRLSCGASGYTYSSYCMGWFRQVPGKEREGVAVIDSDGSTSYADSVKGRFTISKDNGKNTLYLQMNSLKPEDTAMYYCAADLGHYRPPCGVLYLGMDYWGKGTQVTVSSGGGSQVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSASHHHHHH

SEQ ID NO: 297 - protein, 260 aa, synthetic construct
  source 1..260: note = Description of Artificial Sequence: Synthetic polypeptide
QVQLQESGGGSVQAGGSLRLSCGASGYTYSSYCMGWFRQVPGKEREGVAVIDSDGSTSYADSVKGRFTISKDNGKNTLYLQMNSLKPEDTAMYYCAADLGHYRPPCGVLYLGMDYWGKGTQVTVSSGGGSQVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSSASHHHHHH

SEQ ID NO: 298 - protein, 265 aa, synthetic construct
  source 1..265: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSQVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSSASHHHHHH

SEQ ID NO: 299 - protein, 263 aa, synthetic construct
  source 1..263: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSQVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSSASHHHHHH

SEQ ID NO: 300 - protein, 260 aa, synthetic construct
  source 1..260: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSQVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSASHHHHHH

SEQ ID NO: 301 - protein, 264 aa, synthetic construct
  source 1..264: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSQVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSSASHHHHHH

SEQ ID NO: 302 - protein, 265 aa, synthetic construct
  source 1..265: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSQVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSASHHHHHH

SEQ ID NO: 303 - protein, 263 aa, synthetic construct
  source 1..263: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSQVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSASHHHHHH

SEQ ID NO: 304 - protein, 261 aa, synthetic construct
  source 1..261: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSQVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSSASHHHHHH

SEQ ID NO: 305 - protein, 263 aa, synthetic construct
  source 1..263: note = Description of Artificial Sequence: Synthetic polypeptide
QVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSSGGGSQVQLQESGGGSVQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSSASHHHHHH

SEQ ID NO: 306 - protein, 267 aa, synthetic construct
  source 1..267: note = Description of Artificial Sequence: Synthetic polypeptide
QVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSSGGGSQVQLQESGGGSVQAGGSLRLSCAASRFTYSSYCMGWFRQAPGKEREGVASIDSDGSTSYTDSVKGRFTISKDNAKNTLYLQMNSLKPEDTAMYYCALDLMSTVVPGFCGFLLSAGMDYWGKGTQVTVSSASHHHHHH

SEQ ID NO: 307 - protein, 269 aa, synthetic construct
  source 1..269: note = Description of Artificial Sequence: Synthetic polypeptide
QVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSSGGGSQVQLQESGGGSVQAGGSLRLSCAASGYTYSMYCMGWFRQAPGKEREGVAQINSDGSTSYADSVKGRFTISKDNAKNTLYLQMNSLKPEDTAMYYCAADSRVYGGSWYERLCGPYTYEYNYWGQGTQVTVSSASHHHHHH

SEQ ID NO: 308 - protein, 272 aa, synthetic construct
  source 1..272: note = Description of Artificial Sequence: Synthetic polypeptide
QVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSSGGGSQVQLQESGGGSVQAGGSLRLSCAVSGYAYSTYCMGWFRQAPGKEREGVAAIDSGGSTSYADSVKGRFTISKDNAKNTLYLRMNSLKPEDTAMYYCAAVPPPPDGGSCLFLGPEIKVSKADFRYWGQGTQVTVSSASHHHHHH

SEQ ID NO: 309 - protein, 265 aa, synthetic construct
  source 1..265: note = Description of Artificial Sequence: Synthetic polypeptide
QVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSSGGGSQVQLQESGGGSVQAGGSLRLSCTVSGYTYSSNCMGWFRQAPGKEREGVATIYTGGGNTYYADSVKGRFTISQDNAKNTVYLQMNNLKPEDTAMYYCAAEPLSRVYGGSCPTPTFDYWGQGTQVTVSSASHHHHHH

SEQ ID NO: 310 - protein, 264 aa, synthetic construct
  source 1..264: note = Description of Artificial Sequence: Synthetic polypeptide
QVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSSGGGSQVQLQESGGGSVQAGGSLRLSCGASGYTYSSYCMGWFRQVPGKEREGVAVIDSDGSTSYADSVKGRFTISKDNGKNTLYLQMNSLKPEDTAMYYCAADLGHYRPPCGVLYLGMDYWGKGTQVTVSSASHHHHHH

SEQ ID NO: 311 - protein, 265 aa, synthetic construct
  source 1..265: note = Description of Artificial Sequence: Synthetic polypeptide
QVQLQESGGGSVQAGGSLRLSCAASGYTYSSGCMGWFRQAPGKEREAVAAINSDGSTSYADSVKGRFTISKDNAKNTLYLQMNSLKPEDTAMYYCAAEPYCSGGYPRWSVAEFGYWGQGTQVTVSSGGGSQVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSASHHHHHH

SEQ ID NO: 312 - protein, 261 aa, synthetic construct
  source 1..261: note = Description of Artificial Sequence: Synthetic polypeptide
QVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSSGGGSQVQLQESGGGSVQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSSASHHHHHH

SEQ ID NO: 313 - protein, 265 aa, synthetic construct
  source 1..265: note = Description of Artificial Sequence: Synthetic polypeptide
QVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSSGGGSQVQLQESGGGSVQAGGSLRLSCAASRFTYSSYCMGWFRQAPGKEREGVASIDSDGSTSYTDSVKGRFTISKDNAKNTLYLQMNSLKPEDTAMYYCALDLMSTVVPGFCGFLLSAGMDYWGKGTQVTVSSASHHHHHH

SEQ ID NO: 314 - protein, 267 aa, synthetic construct
  source 1..267: note = Description of Artificial Sequence: Synthetic polypeptide
QVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSSGGGSQVQLQESGGGSVQAGGSLRLSCAASGYTYSMYCMGWFRQAPGKEREGVAQINSDGSTSYADSVKGRFTISKDNAKNTLYLQMNSLKPEDTAMYYCAADSRVYGGSWYERLCGPYTYEYNYWGQGTQVTVSSASHHHHHH

SEQ ID NO: 315 - protein, 270 aa, synthetic construct
  source 1..270: note = Description of Artificial Sequence: Synthetic polypeptide
QVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSSGGGSQVQLQESGGGSVQAGGSLRLSCAVSGYAYSTYCMGWFRQAPGKEREGVAAIDSGGSTSYADSVKGRFTISKDNAKNTLYLRMNSLKPEDTAMYYCAAVPPPPDGGSCLFLGPEIKVSKADFRYWGQGTQVTVSSASHHHHHH

SEQ ID NO: 316 - protein, 263 aa, synthetic construct
  source 1..263: note = Description of Artificial Sequence: Synthetic polypeptide
QVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSSGGGSQVQLQESGGGSVQAGGSLRLSCTVSGYTYSSNCMGWFRQAPGKEREGVATIYTGGGNTYYADSVKGRFTISQDNAKNTVYLQMNNLKPEDTAMYYCAAEPLSRVYGGSCPTPTFDYWGQGTQVTVSSASHHHHHH

SEQ ID NO: 317 - protein, 262 aa, synthetic construct
  source 1..262: note = Description of Artificial Sequence: Synthetic polypeptide
QVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSSGGGSQVQLQESGGGSVQAGGSLRLSCGASGYTYSSYCMGWFRQVPGKEREGVAVIDSDGSTSYADSVKGRFTISKDNGKNTLYLQMNSLKPEDTAMYYCAADLGHYRPPCGVLYLGMDYWGKGTQVTVSSASHHHHHH

SEQ ID NO: 318 - protein, 263 aa, synthetic construct
  source 1..263: note = Description of Artificial Sequence: Synthetic polypeptide
QVQLQESGGGSVQAGGSLRLSCAASGYTYSSYCMGWFRQAPGKEREGVAHIDSDGSTSYADSVKGRFTISKDNAKNTLYLQMNSLKPEDTAMYYCAADPIPGPGYCDGGPNKYWGQGTQVTVSSGGGSQVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSASHHHHHH

SEQ ID NO: 319 - protein, 258 aa, synthetic construct
  source 1..258: note = Description of Artificial Sequence: Synthetic polypeptide
QVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSGGGSQVQLQESGGGSVQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSSASHHHHHH

SEQ ID NO: 320 - protein, 262 aa, synthetic construct
  source 1..262: note = Description of Artificial Sequence: Synthetic polypeptide
QVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSGGGSQVQLQESGGGSVQAGGSLRLSCAASRFTYSSYCMGWFRQAPGKEREGVASIDSDGSTSYTDSVKGRFTISKDNAKNTLYLQMNSLKPEDTAMYYCALDLMSTVVPGFCGFLLSAGMDYWGKGTQVTVSSASHHHHHH

SEQ ID NO: 321 - protein, 264 aa, synthetic construct
  source 1..264: note = Description of Artificial Sequence: Synthetic polypeptide
QVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSGGGSQVQLQESGGGSVQAGGSLRLSCAASGYTYSMYCMGWFRQAPGKEREGVAQINSDGSTSYADSVKGRFTISKDNAKNTLYLQMNSLKPEDTAMYYCAADSRVYGGSWYERLCGPYTYEYNYWGQGTQVTVSSASHHHHHH

SEQ ID NO: 322 - protein, 267 aa, synthetic construct
  source 1..267: note = Description of Artificial Sequence: Synthetic polypeptide
QVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSGGGSQVQLQESGGGSVQAGGSLRLSCAVSGYAYSTYCMGWFRQAPGKEREGVAAIDSGGSTSYADSVKGRFTISKDNAKNTLYLRMNSLKPEDTAMYYCAAVPPPPDGGSCLFLGPEIKVSKADFRYWGQGTQVTVSSASHHHHHH

SEQ ID NO: 323 - protein, 260 aa, synthetic construct
  source 1..260: note = Description of Artificial Sequence: Synthetic polypeptide
QVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSGGGSQVQLQESGGGSVQAGGSLRLSCTVSGYTYSSNCMGWFRQAPGKEREGVATIYTGGGNTYYADSVKGRFTISQDNAKNTVYLQMNNLKPEDTAMYYCAAEPLSRVYGGSCPTPTFDYWGQGTQVTVSSASHHHHHH

SEQ ID NO: 324 - protein, 259 aa, synthetic construct
  source 1..259: note = Description of Artificial Sequence: Synthetic polypeptide
QVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSGGGSQVQLQESGGGSVQAGGSLRLSCGASGYTYSSYCMGWFRQVPGKEREGVAVIDSDGSTSYADSVKGRFTISKDNGKNTLYLQMNSLKPEDTAMYYCAADLGHYRPPCGVLYLGMDYWGKGTQVTVSSASHHHHHH

SEQ ID NO: 325 - protein, 260 aa, synthetic construct
  source 1..260: note = Description of Artificial Sequence: Synthetic polypeptide
QVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSGGGSQVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSASHHHHHH

SEQ ID NO: 326 - protein, 262 aa, synthetic construct
  source 1..262: note = Description of Artificial Sequence: Synthetic polypeptide
QVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSSGGGSQVQLQESGGGSVQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSSASHHHHHH

SEQ ID NO: 327 - protein, 266 aa, synthetic construct
  source 1..266: note = Description of Artificial Sequence: Synthetic polypeptide
QVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSSGGGSQVQLQESGGGSVQAGGSLRLSCAASRFTYSSYCMGWFRQAPGKEREGVASIDSDGSTSYTDSVKGRFTISKDNAKNTLYLQMNSLKPEDTAMYYCALDLMSTVVPGFCGFLLSAGMDYWGKGTQVTVSSASHHHHHH

SEQ ID NO: 328 - protein, 268 aa, synthetic construct
  source 1..268: note = Description of Artificial Sequence: Synthetic polypeptide
QVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSSGGGSQVQLQESGGGSVQAGGSLRLSCAASGYTYSMYCMGWFRQAPGKEREGVAQINSDGSTSYADSVKGRFTISKDNAKNTLYLQMNSLKPEDTAMYYCAADSRVYGGSWYERLCGPYTYEYNYWGQGTQVTVSSASHHHHHH

SEQ ID NO: 329 - protein, 271 aa, synthetic construct
  source 1..271: note = Description of Artificial Sequence: Synthetic polypeptide
QVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSSGGGSQVQLQESGGGSVQAGGSLRLSCAVSGYAYSTYCMGWFRQAPGKEREGVAAIDSGGSTSYADSVKGRFTISKDNAKNTLYLRMNSLKPEDTAMYYCAAVPPPPDGGSCLFLGPEIKVSKADFRYWGQGTQVTVSSASHHHHHH

SEQ ID NO: 330 - protein, 264 aa, synthetic construct
  source 1..264: note = Description of Artificial Sequence: Synthetic polypeptide
QVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSSGGGSQVQLQESGGGSVQAGGSLRLSCTVSGYTYSSNCMGWFRQAPGKEREGVATIYTGGGNTYYADSVKGRFTISQDNAKNTVYLQMNNLKPEDTAMYYCAAEPLSRVYGGSCPTPTFDYWGQGTQVTVSSASHHHHHH

SEQ ID NO: 331 - protein, 263 aa, synthetic construct
  source 1..263: note = Description of Artificial Sequence: Synthetic polypeptide
QVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSSGGGSQVQLQESGGGSVQAGGSLRLSCGASGYTYSSYCMGWFRQVPGKEREGVAVIDSDGSTSYADSVKGRFTISKDNGKNTLYLQMNSLKPEDTAMYYCAADLGHYRPPCGVLYLGMDYWGKGTQVTVSSASHHHHHH

SEQ ID NO: 332 - protein, 264 aa, synthetic construct
  source 1..264: note = Description of Artificial Sequence: Synthetic polypeptide
QVQLQESGGGSVQAGGSLRLSCTVSRYTASVNYMGWFRQAPGKEREGVATIFTGAGTTYYANSVKGRFTISRDNAKNTAYLQMNSLKPEDTAIYYCAVDFRGGLLYRPAYEYTYRGQGTQVTVSSGGGSQVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSASHHHHHH

SEQ ID NO: 333 - protein, 263 aa, synthetic construct
  source 1..263: note = Description of Artificial Sequence: Synthetic polypeptide
QVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSGGGSQVQLQESGGGSVQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSSASHHHHHH

SEQ ID NO: 334 - protein, 267 aa, synthetic construct
  source 1..267: note = Description of Artificial Sequence: Synthetic polypeptide
QVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSGGGSQVQLQESGGGSVQAGGSLRLSCAASRFTYSSYCMGWFRQAPGKEREGVASIDSDGSTSYTDSVKGRFTISKDNAKNTLYLQMNSLKPEDTAMYYCALDLMSTVVPGFCGFLLSAGMDYWGKGTQVTVSSASHHHHHH

SEQ ID NO: 335 - protein, 269 aa, synthetic construct
  source 1..269: note = Description of Artificial Sequence: Synthetic polypeptide
QVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSGGGSQVQLQESGGGSVQAGGSLRLSCAASGYTYSMYCMGWFRQAPGKEREGVAQINSDGSTSYADSVKGRFTISKDNAKNTLYLQMNSLKPEDTAMYYCAADSRVYGGSWYERLCGPYTYEYNYWGQGTQVTVSSASHHHHHH

SEQ ID NO: 336 - protein, 272 aa, synthetic construct
  source 1..272: note = Description of Artificial Sequence: Synthetic polypeptide
QVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSGGGSQVQLQESGGGSVQAGGSLRLSCAVSGYAYSTYCMGWFRQAPGKEREGVAAIDSGGSTSYADSVKGRFTISKDNAKNTLYLRMNSLKPEDTAMYYCAAVPPPPDGGSCLFLGPEIKVSKADFRYWGQGTQVTVSSASHHHHHH

SEQ ID NO: 337 - protein, 265 aa, synthetic construct
  source 1..265: note = Description of Artificial Sequence: Synthetic polypeptide
QVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSGGGSQVQLQESGGGSVQAGGSLRLSCTVSGYTYSSNCMGWFRQAPGKEREGVATIYTGGGNTYYADSVKGRFTISQDNAKNTVYLQMNNLKPEDTAMYYCAAEPLSRVYGGSCPTPTFDYWGQGTQVTVSSASHHHHHH

SEQ ID NO: 338 - protein, 264 aa, synthetic construct
  source 1..264: note = Description of Artificial Sequence: Synthetic polypeptide
QVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSGGGSQVQLQESGGGSVQAGGSLRLSCGASGYTYSSYCMGWFRQVPGKEREGVAVIDSDGSTSYADSVKGRFTISKDNGKNTLYLQMNSLKPEDTAMYYCAADLGHYRPPCGVLYLGMDYWGKGTQVTVSSASHHHHHH

SEQ ID NO: 339 - protein, 265 aa, synthetic construct
  source 1..265: note = Description of Artificial Sequence: Synthetic polypeptide
QVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSGGGSQVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSASHHHHHH

SEQ ID NO: 340 - protein, 261 aa, synthetic construct
  source 1..261: note = Description of Artificial Sequence: Synthetic polypeptide
QVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSGGGSQVQLQESGGGSVQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSSASHHHHHH

SEQ ID NO: 341 - protein, 265 aa, synthetic construct
  source 1..265: note = Description of Artificial Sequence: Synthetic polypeptide
QVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSGGGSQVQLQESGGGSVQAGGSLRLSCAASRFTYSSYCMGWFRQAPGKEREGVASIDSDGSTSYTDSVKGRFTISKDNAKNTLYLQMNSLKPEDTAMYYCALDLMSTVVPGFCGFLLSAGMDYWGKGTQVTVSSASHHHHHH

SEQ ID NO: 342 - protein, 267 aa, synthetic construct
  source 1..267: note = Description of Artificial Sequence: Synthetic polypeptide
QVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSGGGSQVQLQESGGGSVQAGGSLRLSCAASGYTYSMYCMGWFRQAPGKEREGVAQINSDGSTSYADSVKGRFTISKDNAKNTLYLQMNSLKPEDTAMYYCAADSRVYGGSWYERLCGPYTYEYNYWGQGTQVTVSSASHHHHHH

SEQ ID NO: 343 - protein, 270 aa, synthetic construct
  source 1..270: note = Description of Artificial Sequence: Synthetic polypeptide
QVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSGGGSQVQLQESGGGSVQAGGSLRLSCAVSGYAYSTYCMGWFRQAPGKEREGVAAIDSGGSTSYADSVKGRFTISKDNAKNTLYLRMNSLKPEDTAMYYCAAVPPPPDGGSCLFLGPEIKVSKADFRYWGQGTQVTVSSASHHHHHH

SEQ ID NO: 344 - protein, 263 aa, synthetic construct
  source 1..263: note = Description of Artificial Sequence: Synthetic polypeptide
QVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSGGGSQVQLQESGGGSVQAGGSLRLSCTVSGYTYSSNCMGWFRQAPGKEREGVATIYTGGGNTYYADSVKGRFTISQDNAKNTVYLQMNNLKPEDTAMYYCAAEPLSRVYGGSCPTPTFDYWGQGTQVTVSSASHHHHHH

SEQ ID NO: 345 - protein, 262 aa, synthetic construct
  source 1..262: note = Description of Artificial Sequence: Synthetic polypeptide
QVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSGGGSQVQLQESGGGSVQAGGSLRLSCGASGYTYSSYCMGWFRQVPGKEREGVAVIDSDGSTSYADSVKGRFTISKDNGKNTLYLQMNSLKPEDTAMYYCAADLGHYRPPCGVLYLGMDYWGKGTQVTVSSASHHHHHH

SEQ ID NO: 346 - protein, 263 aa, synthetic construct
  source 1..263: note = Description of Artificial Sequence: Synthetic polypeptide
QVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSGGGSQVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSASHHHHHH

SEQ ID NO: 347 - protein, 259 aa, synthetic construct
  source 1..259: note = Description of Artificial Sequence: Synthetic polypeptide
QVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSSGGGSQVQLQESGGGSVQAGGSLRLSCAASRYLYSIDYMAWFRQSPGKEREPVAVIYTASGATFYPDSVKGRFTISQDNAKMTVYLQMNSLKSEDTAMYYCAAVRKTDSYLFDAQSFTYWGQGTQVTVSSASHHHHHH

SEQ ID NO: 348 - protein, 263 aa, synthetic construct
  source 1..263: note = Description of Artificial Sequence: Synthetic polypeptide
QVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSSGGGSQVQLQESGGGSVQAGGSLRLSCAASRFTYSSYCMGWFRQAPGKEREGVASIDSDGSTSYTDSVKGRFTISKDNAKNTLYLQMNSLKPEDTAMYYCALDLMSTVVPGFCGFLLSAGMDYWGKGTQVTVSSASHHHHHH

SEQ ID NO: 349 - protein, 265 aa, synthetic construct
  source 1..265: note = Description of Artificial Sequence: Synthetic polypeptide
QVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSSGGGSQVQLQESGGGSVQAGGSLRLSCAASGYTYSMYCMGWFRQAPGKEREGVAQINSDGSTSYADSVKGRFTISKDNAKNTLYLQMNSLKPEDTAMYYCAADSRVYGGSWYERLCGPYTYEYNYWGQGTQVTVSSASHHHHHH

SEQ ID NO: 350 - protein, 268 aa, synthetic construct
  source 1..268: note = Description of Artificial Sequence: Synthetic polypeptide
QVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSSGGGSQVQLQESGGGSVQAGGSLRLSCAVSGYAYSTYCMGWFRQAPGKEREGVAAIDSGGSTSYADSVKGRFTISKDNAKNTLYLRMNSLKPEDTAMYYCAAVPPPPDGGSCLFLGPEIKVSKADFRYWGQGTQVTVSSASHHHHHH

SEQ ID NO: 351 - protein, 261 aa, synthetic construct
  source 1..261: note = Description of Artificial Sequence: Synthetic polypeptide
QVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSSGGGSQVQLQESGGGSVQAGGSLRLSCTVSGYTYSSNCMGWFRQAPGKEREGVATIYTGGGNTYYADSVKGRFTISQDNAKNTVYLQMNNLKPEDTAMYYCAAEPLSRVYGGSCPTPTFDYWGQGTQVTVSSASHHHHHH

SEQ ID NO: 352 - protein, 260 aa, synthetic construct
  source 1..260: note = Description of Artificial Sequence: Synthetic polypeptide
QVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSSGGGSQVQLQESGGGSVQAGGSLRLSCGASGYTYSSYCMGWFRQVPGKEREGVAVIDSDGSTSYADSVKGRFTISKDNGKNTLYLQMNSLKPEDTAMYYCAADLGHYRPPCGVLYLGMDYWGKGTQVTVSSASHHHHHH

SEQ ID NO: 353 - protein, 261 aa, synthetic construct
  source 1..261: note = Description of Artificial Sequence: Synthetic polypeptide
QVQLQESGGGSVQTGGSLRLSCAASGYTYLRGCMGWFRQAPGKEREGVAVMDVVGDRRSYIDSVKGRFTISRDNAANSVYLQMDNLKPEDTAMYYCTAGPNCVGWRSGLDYWGQGTQVTVSSGGGSQVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSASHHHHHH

SEQ ID NO: 354 - DNA, 789 bp, synthetic construct
  source 1..789: mol_type = other DNA; note = Description of Artificial Sequence: Synthetic polynucleotide
caagtgcagctgcaagagagcggaggaggaagcgtgcaagccggaggctctctgagactgagctgtgccgcctctaggtatctgtacagcatcgactacatggcttggttcagacagagccccggcaaggagagggagccagtggctgtcatctacactgcctccggcgccacattctatccagatagcgtgaagggaaggttcactatcagccaagataacgccaagatgacagtgtatctgcagatgaactctctgaagagcgaggacactgccatgtactactgtgccgccgtgaggaagacagatagctacctcttcgacgcccagagcttcacatactggggccaaggcacacaagtgacagtctcgagcggcggaggatcccaagtgcagctgcaagagagcggcggaggaagcgtgcaagctggaggctctctgaggctgagctgtgctgccagcggctacacttatagcagcggctgtatgggctggttcagacaagcccccggcaaggaaagggaagccgtggccgccatcaattccgatggcagcacaagctacgccgacagcgtgaagggaaggttcacaatcagcaaggacaacgccaagaacacactctatctgcagatgaactctctgaagccagaggacacagccatgtactactgcgccgctgagccttactgtagcggcggctacccaagatggagcgtcgctgagttcggctactggggccaaggcacacaagtgactgtctcgtctgctagccaccatcaccatcaccac

SEQ ID NO: 355 - DNA, 783 bp, synthetic construct
  source 1..783: mol_type = other DNA; note = Description of Artificial Sequence: Synthetic polynucleotide
caagtgcagctgcaagagagcggaggaggaagcgtgcaagccggaggctctctgagactgagctgtgccgcctctaggtatctgtacagcatcgactacatggcttggttcagacagagccccggcaaggagagggagccagtggctgtcatctacactgcctccggcgccacattctatccagatagcgtgaagggaaggttcactatcagccaagataacgccaagatgacagtgtatctgcagatgaactctctgaagagcgaggacactgccatgtactactgtgccgccgtgaggaagacagatagctacctcttcgacgcccagagcttcacatactggggccaaggcacacaagtgacagtctcgagcggcggaggatcccaagtgcagctgcaagagagcggaggaggcagcgtccaagccggaggctctctgaggctgagctgtgctgccagcggctacacttacagcagctactgcatgggctggttcagacaagcccccggcaaggagagagagggcgtggctcacatcgacagcgacggctccacaagctacgccgatagcgtgaagggaaggttcacaatctccaaggacaacgccaagaacactctgtacctccagatgaactctctgaagccagaggacactgccatgtactactgtgccgccgatccaattcccggccccggctactgcgatggcggccctaacaagtactggggccaaggcacacaagtgactgtctcgtctgctagccaccatcaccatcaccac

SEQ ID NO: 356 - DNA, 774 bp, synthetic construct
  source 1..774: mol_type = other DNA; note = Description of Artificial Sequence: Synthetic polynucleotide
caagtgcagctgcaagagagcggaggaggaagcgtgcaagccggaggctctctgagactgagctgtgccgcctctaggtatctgtacagcatcgactacatggcttggttcagacagagccccggcaaggagagggagccagtggctgtcatctacactgcctccggcgccacattctatccagatagcgtgaagggaaggttcactatcagccaagataacgccaagatgacagtgtatctgcagatgaactctctgaagagcgaggacactgccatgtactactgtgccgccgtgaggaagacagatagctacctcttcgacgcccagagcttcacatactggggccaaggcacacaagtgacagtctcgagcggcggaggatcccaagtgcagctgcaagagtccggaggaggaagcgtgcaagccggcggatctctgagactgagctgtgccgcctctaggtacacttacaacagctactgcatgggctggttcagacaagcccccggcaaggaaagagagggcgtggccactatcgatagcgacggcatgactaggtacgctgatagcgtcaagggaaggttcacaatctccaaggacaatgctaagaacactctgtacctccagatgaactctctgaagccagaggacacagccatgtactactgcgctgccgatgccgactgcactatcgccgccatgactactaatcctctgggccaaggcacacaagtgactgtctcgtctgctagccaccatcaccatcaccac

SEQ ID NO: 357 - DNA, 786 bp, synthetic construct
  source 1..786: mol_type = other DNA; note = Description of Artificial Sequence: Synthetic polynucleotide
caagtgcagctgcaagagagcggaggaggaagcgtgcaagccggaggctctctgagactgagctgtgccgcctctaggtatctgtacagcatcgactacatggcttggttcagacagagccccggcaaggagagggagccagtggctgtcatctacactgcctccggcgccacattctatccagatagcgtgaagggaaggttcactatcagccaagataacgccaagatgacagtgtatctgcagatgaactctctgaagagcgaggacactgccatgtactactgtgccgccgtgaggaagacagatagctacctcttcgacgcccagagcttcacatactggggccaaggcacacaagtgacagtctcgagcggcggaggatcccaagtgcagctgcaagagtccggcggaggcagcgtccaagccggaggatctctgaggctgagctgtacagtgagcagatacactgccagcgtgaactacatgggctggttcagacaagcccccggcaaagagagagagggcgtggccacaatcttcactggcgccggcacaacatactacgccaactccgtcaagggaaggttcacaatctctagggacaacgccaagaacactgcctatctgcagatgaactccctcaagccagaggacactgccatctactactgcgccgtggatttcagaggcggactgctgtataggccagcctacgagtacacttataggggccaaggcacacaagtgacagtctcgtctgctagccaccatcaccatcaccac

SEQ ID NO: 358 - DNA, 789 bp, synthetic construct
  source 1..789: mol_type = other DNA; note = Description of Artificial Sequence: Synthetic polynucleotide
caagtgcagctgcaagagagcggaggaggaagcgtgcaagccggaggctctctgagactgagctgtgccgcctctaggtatctgtacagcatcgactacatggcttggttcagacagagccccggcaaggagagggagccagtggctgtcatctacactgcctccggcgccacattctatccagatagcgtgaagggaaggttcactatcagccaagataacgccaagatgacagtgtatctgcagatgaactctctgaagagcgaggacactgccatgtactactgtgccgccgtgaggaagacagatagctacctcttcgacgcccagagcttcacatactggggccaaggcacacaagtgacagtctcgagcggcggaggatcccaagtgcagctgcaagagagcggaggaggcagcgtcgaagctggaggatctctgaggctgagctgtgctgccagcggctacactcacagcagctactgtatgggctggttcagacaagcccccggcaaggagagggaaggcgtggctgccatcgacgtggatggcagcactacttacgccgacagcgtgaagggaaggttcactatcagcaaggacaacgccaagaacacactctatctgcagatgaacagcctcaagccagaggacactggcatgtactactgcgccgccgagttcgccgattgcagcagcaactactttctgcctcccggcgccgtcagatattggggccaaggcactcaagtgacagtctcgtctgctagccaccatcaccatcaccac

SEQ ID NO: 359 - DNA, 783 bp, synthetic construct
  source 1..783: mol_type = other DNA; note = Description of Artificial Sequence: Synthetic polynucleotide
caagtgcagctgcaagagagcggaggaggaagcgtgcaagccggaggctctctgagactgagctgtgccgcctctaggtatctgtacagcatcgactacatggcttggttcagacagagccccggcaaggagagggagccagtggctgtcatctacactgcctccggcgccacattctatccagatagcgtgaagggaaggttcactatcagccaagataacgccaagatgacagtgtatctgcagatgaactctctgaagagcgaggacactgccatgtactactgtgccgccgtgaggaagacagatagctacctcttcgacgcccagagcttcacatactggggccaaggcacacaagtgacagtctcgagcggcggaggatcccaagtgcagctgcaagagagcggaggaggaagcgtccaagccggaggatctctgagactgagctgcgccgctagtggctactcctacagcagctactgcatgggctggtttaggcaagcccccggcaaggagagagaaggcgtggccactatcgacagcgacggcatgacaaggtacgccgacagcgtgaagggaaggttcacaatcagcaaggacaacgccaagaacacactgtatctgcagatgaactctctgaagccagaggacactgccatgtactactgtgccgctcctctgtacgactgtgatagcggcgctgtgggcagaaatccaccttattggggccaaggcactcaagtgacagtctcgtctgctagccaccatcaccatcaccac

SEQ ID NO: 360 - DNA, 777 bp, synthetic construct
  source 1..777: mol_type = other DNA; note = Description of Artificial Sequence: Synthetic polynucleotide
caagtgcagctgcaagagagcggaggaggaagcgtgcaagccggaggctctctgagactgagctgtgccgcctctaggtatctgtacagcatcgactacatggcttggttcagacagagccccggcaaggagagggagccagtggctgtcatctacactgcctccggcgccacattctatccagatagcgtgaagggaaggttcactatcagccaagataacgccaagatgacagtgtatctgcagatgaactctctgaagagcgaggacactgccatgtactactgtgccgccgtgaggaagacagatagctacctcttcgacgcccagagcttcacatactggggccaaggcacacaagtgacagtctcgagcggcggaggatcccaagtgcagctgcaagagagcggaggcggcagcgtgcagactggaggctctctgagactgagctgtgctgccagcggctacacttatctgaggggctgtatgggctggtttaggcaagcccccggcaaggagagagagggcgtggccgtcatggatgtggtgggcgataggagaagctacatcgacagcgtgaagggaaggttcacaatctctagggacaatgccgccaacagcgtctatctgcagatggacaatctgaagccagaggacacagccatgtactactgcactgccggccctaactgtgtgggctggagaagcggactggattactggggccaaggcacacaagtgacagtctcgtctgctagccaccatcaccatcaccac

SEQ ID NO: 361 - DNA, 801 bp, synthetic construct
  source 1..801: mol_type = other DNA; note = Description of Artificial Sequence: Synthetic polynucleotide
caagtgcagctgcaagagagcggaggaggaagcgtgcaagccggaggctctctgaggctgagctgtgccgcctctaggttcacatacagcagctactgcatgggctggttcagacaagcccccggcaaagagagagaaggcgtggccagcatcgatagcgatggctccactagctacactgacagcgtgaagggaaggttcactatcagcaaggacaacgccaagaacactctgtatctgcagatgaactctctgaagccagaggacacagccatgtactactgtgccctcgatctgatgagcacagtggtgcccggcttctgtggctttctgctgagcgctggcatggattactggggcaagggcactcaagtgactgtctcgagcggcggaggatcccaagtgcagctgcaagagagcggcggaggaagcgtgcaagctggaggctctctgaggctgagctgtgctgccagcggctacacttatagcagcggctgtatgggctggttcagacaagcccccggcaaggaaagggaagccgtggccgccatcaattccgatggcagcacaagctacgccgacagcgtgaagggaaggttcacaatcagcaaggacaacgccaagaacacactctatctgcagatgaactctctgaagccagaggacacagccatgtactactgcgccgctgagccttactgtagcggcggctacccaagatggagcgtcgctgagttcggctactggggccaaggcacacaagtgactgtctcgtctgctagccaccatcaccatcaccac

SEQ ID NO: 362 - DNA, 795 bp, synthetic construct
  source 1..795: mol_type = other DNA; note = Description of Artificial Sequence: Synthetic polynucleotide
caagtgcagctgcaagagagcggaggaggaagcgtgcaagccggaggctctctgaggctgagctgtgccgcctctaggttcacatacagcagctactgcatgggctggttcagacaagcccccggcaaagagagagaaggcgtggccagcatcgatagcgatggctccactagctacactgacagcgtgaagggaaggttcactatcagcaaggacaacgccaagaacactctgtatctgcagatgaactctctgaagccagaggacacagccatgtactactgtgccctcgatctgatgagcacagtggtgcccggcttctgtggctttctgctgagcgctggcatggattactggggcaagggcactcaagtgactgtctcgagcggcggaggatcccaagtgcagctgcaagagagcggaggaggcagcgtccaagccggaggctctctgaggctgagctgtgctgccagcggctacacttacagcagctactgcatgggctggttcagacaagcccccggcaaggagagagagggcgtggctcacatcgacagcgacggctccacaagctacgccgatagcgtgaagggaaggttcacaatctccaaggacaacgccaagaacactctgtacctccagatgaactctctgaagccagaggacactgccatgtactactgtgccgccgatccaattcccggccccggctactgcgatggcggccctaacaagtactggggccaaggcacacaagtgactgtctcgtctgctagccaccatcaccatcaccac

SEQ ID NO: 363 - DNA, 786 bp, synthetic construct
  source 1..786: mol_type = other DNA; note = Description of Artificial Sequence: Synthetic polynucleotide
caagtgcagctgcaagagagcggaggaggaagcgtgcaagccggaggctctctgaggctgagctgtgccgcctctaggttcacatacagcagctactgcatgggctggttcagacaagcccccggcaaagagagagaaggcgtggccagcatcgatagcgatggctccactagctacactgacagcgtgaagggaaggttcactatcagcaaggacaacgccaagaacactctgtatctgcagatgaactctctgaagccagaggacacagccatgtactactgtgccctcgatctgatgagcacagtggtgcccggcttctgtggctttctgctgagcgctggcatggattactggggcaagggcactcaagtgactgtctcgagcggcggaggatcccaagtgcagctgcaagagtccggaggaggaagcgtgcaagccggcggatctctgagactgagctgtgccgcctctaggtacacttacaacagctactgcatgggctggttcagacaagcccccggcaaggaaagagagggcgtggccactatcgatagcgacggcatgactaggtacgctgatagcgtcaagggaaggttcacaatctccaaggacaatgctaagaacactctgtacctccagatgaactctctgaagccagaggacacagccatgtactactgcgctgccgatgccgactgcactatcgccgccatgactactaatcctctgggccaaggcacacaagtgactgtctcgtctgctagccaccatcaccatcaccac

SEQ ID NO: 364 - DNA, 798 bp, synthetic construct
  source 1..798: mol_type = other DNA; note = Description of Artificial Sequence: Synthetic polynucleotide
caagtgcagctgcaagagagcggaggaggaagcgtgcaagccggaggctctctgaggctgagctgtgccgcctctaggttcacatacagcagctactgcatgggctggttcagacaagcccccggcaaagagagagaaggcgtggccagcatcgatagcgatggctccactagctacactgacagcgtgaagggaaggttcactatcagcaaggacaacgccaagaacactctgtatctgcagatgaactctctgaagccagaggacacagccatgtactactgtgccctcgatctgatgagcacagtggtgcccggcttctgtggctttctgctgagcgctggcatggattactggggcaagggcactcaagtgactgtctcgagcggcggaggatcccaagtgcagctgcaagagtccggcggaggcagcgtccaagccggaggatctctgaggctgagctgtacagtgagcagatacactgccagcgtgaactacatgggctggttcagacaagcccccggcaaagagagagagggcgtggccacaatcttcactggcgccggcacaacatactacgccaactccgtcaagggaaggttcacaatctctagggacaacgccaagaacactgcctatctgcagatgaactccctcaagccagaggacactgccatctactactgcgccgtggatttcagaggcggactgctgtataggccagcctacgagtacacttataggggccaaggcacacaagtgacagtctcgtctgctagccaccatcaccatcaccac

SEQ ID NO: 365 - DNA, 801 bp, synthetic construct
  source 1..801: mol_type = other DNA; note = Description of Artificial Sequence: Synthetic polynucleotide
caagtgcagctgcaagagagcggaggaggaagcgtgcaagccggaggctctctgaggctgagctgtgccgcctctaggttcacatacagcagctactgcatgggctggttcagacaagcccccggcaaagagagagaaggcgtggccagcatcgatagcgatggctccactagctacactgacagcgtgaagggaaggttcactatcagcaaggacaacgccaagaacactctgtatctgcagatgaactctctgaagccagaggacacagccatgtactactgtgccctcgatctgatgagcacagtggtgcccggcttctgtggctttctgctgagcgctggcatggattactggggcaagggcactcaagtgactgtctcgagcggcggaggatcccaagtgcagctgcaagagagcggaggaggcagcgtcgaagctggaggatctctgaggctgagctgtgctgccagcggctacactcacagcagctactgtatgggctggttcagacaagcccccggcaaggagagggaaggcgtggctgccatcgacgtggatggcagcactacttacgccgacagcgtgaagggaaggttcactatcagcaaggacaacgccaagaacacactctatctgcagatgaacagcctcaagccagaggacactggcatgtactactgcgccgccgagttcgccgattgcagcagcaactactttctgcctcccggcgccgtcagatattggggccaaggcactcaagtgacagtctcgtctgctagccaccatcaccatcaccac

SEQ ID NO: 366 - DNA, 795 bp, synthetic construct
  source 1..795: mol_type = other DNA; note = Description of Artificial Sequence: Synthetic polynucleotide
caagtgcagctgcaagagagcggaggaggaagcgtgcaagccggaggctctctgaggctgagctgtgccgcctctaggttcacatacagcagctactgcatgggctggttcagacaagcccccggcaaagagagagaaggcgtggccagcatcgatagcgatggctccactagctacactgacagcgtgaagggaaggttcactatcagcaaggacaacgccaagaacactctgtatctgcagatgaactctctgaagccagaggacacagccatgtactactgtgccctcgatctgatgagcacagtggtgcccggcttctgtggctttctgctgagcgctggcatggattactggggcaagggcactcaagtgactgtctcgagcggcggaggatcccaagtgcagctgcaagagagcggaggaggaagcgtccaagccggaggatctctgagactgagctgcgccgctagtggctactcctacagcagctactgcatgggctggtttaggcaagcccccggcaaggagagagaaggcgtggccactatcgacagcgacggcatgacaaggtacgccgacagcgtgaagggaaggttcacaatcagcaaggacaacgccaagaacacactgtatctgcagatgaactctctgaagccagaggacactgccatgtactactgtgccgctcctctgtacgactgtgatagcggcgctgtgggcagaaatccaccttattggggccaaggcactcaagtgacagtctcgtctgctagccaccatcaccatcaccac

SEQ ID NO: 367 - DNA, 789 bp, synthetic construct
  source 1..789: mol_type = other DNA; note = Description of Artificial Sequence: Synthetic polynucleotide
caagtgcagctgcaagagagcggaggaggaagcgtgcaagccggaggctctctgaggctgagctgtgccgcctctaggttcacatacagcagctactgcatgggctggttcagacaagcccccggcaaagagagagaaggcgtggccagcatcgatagcgatggctccactagctacactgacagcgtgaagggaaggttcactatcagcaaggacaacgccaagaacactctgtatctgcagatgaactctctgaagccagaggacacagccatgtactactgtgccctcgatctgatgagcacagtggtgcccggcttctgtggctttctgctgagcgctggcatggattactggggcaagggcactcaagtgactgtctcgagcggcggaggatcccaagtgcagctgcaagagagcggaggcggcagcgtgcagactggaggctctctgagactgagctgtgctgccagcggctacacttatctgaggggctgtatgggctggtttaggcaagcccccggcaaggagagagagggcgtggccgtcatggatgtggtgggcgataggagaagctacatcgacagcgtgaagggaaggttcacaatctctagggacaatgccgccaacagcgtctatctgcagatggacaatctgaagccagaggacacagccatgtactactgcactgccggccctaactgtgtgggctggagaagcggactggattactggggccaaggcacacaagtgacagtctcgtctgctagccaccatcaccatcaccac

SEQ ID NO: 368 - DNA, 807 bp, synthetic construct
  source 1..807: mol_type = other DNA; note = Description of Artificial Sequence: Synthetic polynucleotide
caagtgcagctgcaagagtccggaggaggcagcgtccaagccggaggctctctgaggctgagctgtgctgccagcggctacacttacagcatgtactgcatgggctggttcagacaagcccccggcaaggaaagagagggcgtggcccagatcaatagcgatggcagcacaagctacgccgacagcgtgaagggaaggttcactatctccaaggacaacgccaagaacactctgtatctgcagatgaactctctgaagccagaggacactgccatgtactactgcgctgccgattctagggtgtacggcggcagctggtatgagaggctctgcggcccttacacatacgagtacaactactggggccaaggcacacaagtgactgtctcgagcggcggaggatcccaagtgcagctgcaagagagcggcggaggaagcgtgcaagctggaggctctctgaggctgagctgtgctgccagcggctacacttatagcagcggctgtatgggctggttcagacaagcccccggcaaggaaagggaagccgtggccgccatcaattccgatggcagcacaagctacgccgacagcgtgaagggaaggttcacaatcagcaaggacaacgccaagaacacactctatctgcagatgaactctctgaagccagaggacacagccatgtactactgcgccgctgagccttactgtagcggcggctacccaagatggagcgtcgctgagttcggctactggggccaaggcacacaagtgactgtctcgtctgctagccaccatcaccatcaccac

SEQ ID NO: 369 - DNA, 801 bp, synthetic construct
  source 1..801: mol_type = other DNA; note = Description of Artificial Sequence: Synthetic polynucleotide
caagtgcagctgcaagagtccggaggaggcagcgtccaagccggaggctctctgaggctgagctgtgctgccagcggctacacttacagcatgtactgcatgggctggttcagacaagcccccggcaaggaaagagagggcgtggcccagatcaatagcgatggcagcacaagctacgccgacagcgtgaagggaaggttcactatctccaaggacaacgccaagaacactctgtatctgcagatgaactctctgaagccagaggacactgccatgtactactgcgctgccgattctagggtgtacggcggcagctggtatgagaggctctgcggcccttacacatacgagtacaactactggggccaaggcacacaagtgactgtctcgagcggcggaggatcccaagtgcagctgcaagagagcggaggaggcagcgtccaagccggaggctctctgaggctgagctgtgctgccagcggctacacttacagcagctactgcatgggctggttcagacaagcccccggcaaggagagagagggcgtggctcacatcgacagcgacggctccacaagctacgccgatagcgtgaagggaaggttcacaatctccaaggacaacgccaagaacactctgtacctccagatgaactctctgaagccagaggacactgccatgtactactgtgccgccgatccaattcccggccccggctactgcgatggcggccctaacaagtactggggccaaggcacacaagtgactgtctcgtctgctagccaccatcaccatcaccac

SEQ ID NO: 370 - DNA, 792 bp, synthetic construct
  source 1..792: mol_type = other DNA; note = Description of Artificial Sequence: Synthetic polynucleotide
caagtgcagctgcaagagtccggaggaggcagcgtccaagccggaggctctctgaggctgagctgtgctgccagcggctacacttacagcatgtactgcatgggctggttcagacaagcccccggcaaggaaagagagggcgtggcccagatcaatagcgatggcagcacaagctacgccgacagcgtgaagggaaggttcactatctccaaggacaacgccaagaacactctgtatctgcagatgaactctctgaagccagaggacactgccatgtactactgcgctgccgattctagggtgtacggcggcagctggtatgagaggctctgcggcccttacacatacgagtacaactactggggccaaggcacacaagtgactgtctcgagcggcggaggatcccaagtgcagctgcaagagtccggaggaggaagcgtgcaagccggcggatctctgagactgagctgtgccgcctctaggtacacttacaacagctactgcatgggctggttcagacaagcccccggcaaggaaagagagggcgtggccactatcgatagcgacggcatgactaggtacgctgatagcgtcaagggaaggttcacaatctccaaggacaatgctaagaacactctgtacctccagatgaactctctgaagccagaggacacagccatgtactactgcgctgccgatgccgactgcactatcgccgccatgactactaatcctctgggccaaggcacacaagtgactgtctcgtctgctagccaccatcaccatcaccac

SEQ ID NO: 371 - DNA, 804 bp, synthetic construct
  source 1..804: mol_type = other DNA; note = Description of Artificial Sequence: Synthetic polynucleotide
caagtgcagctgcaagagtccggaggaggcagcgtccaagccggaggctctctgaggctgagctgtgctgccagcggctacacttacagcatgtactgcatgggctggttcagacaagcccccggcaaggaaagagagggcgtggcccagatcaatagcgatggcagcacaagctacgccgacagcgtgaagggaaggttcactatctccaaggacaacgccaagaacactctgtatctgcagatgaactctctgaagccagaggacactgccatgtactactgcgctgccgattctagggtgtacggcggcagctggtatgagaggctctgcggcccttacacatacgagtacaactactggggccaaggcacacaagtgactgtctcgagcggcggaggatcccaagtgcagctgcaagagtccggcggaggcagcgtccaagccggaggatctctgaggctgagctgtacagtgagcagatacactgccagcgtgaactacatgggctggttcagacaagcccccggcaaagagagagagggcgtggccacaatcttcactggcgccggcacaacatactacgccaactccgtcaagggaaggttcacaatctctagggacaacgccaagaacactgcctatctgcagatgaactccctcaagccagaggacactgccatctactactgcgccgtggatttcagaggcggactgctgtataggccagcctacgagtacacttataggggccaaggcacacaagtgacagtctcgtctgctagccaccatcaccatcaccac

SEQ ID NO: 372 - DNA, 807 bp, synthetic construct
  source 1..807: mol_type = other DNA; note = Description of Artificial Sequence: Synthetic polynucleotide
caagtgcagctgcaagagtccggaggaggcagcgtccaagccggaggctctctgaggctgagctgtgctgccagcggctacacttacagcatgtactgcatgggctggttcagacaagcccccggcaaggaaagagagggcgtggcccagatcaatagcgatggcagcacaagctacgccgacagcgtgaagggaaggttcactatctccaaggacaacgccaagaacactctgtatctgcagatgaactctctgaagccagaggacactgccatgtactactgcgctgccgattctagggtgtacggcggcagctggtatgagaggctctgcggcccttacacatacgagtacaactactggggccaaggcacacaagtgactgtctcgagcggcggaggatcccaagtgcagctgcaagagagcggaggaggcagcgtcgaagctggaggatctctgaggctgagctgtgctgccagcggctacactcacagcagctactgtatgggctggttcagacaagcccccggcaaggagagggaaggcgtggctgccatcgacgtggatggcagcactacttacgccgacagcgtgaagggaaggttcactatcagcaaggacaacgccaagaacacactctatctgcagatgaacagcctcaagccagaggacactggcatgtactactgcgccgccgagttcgccgattgcagcagcaactactttctgcctcccggcgccgtcagatattggggccaaggcactcaagtgacagtctcgtctgctagccaccatcaccatcaccac

SEQ ID NO: 373 - DNA, 801 bp, synthetic construct
  source 1..801: mol_type = other DNA; note = Description of Artificial Sequence: Synthetic polynucleotide
caagtgcagctgcaagagtccggaggaggcagcgtccaagccggaggctctctgaggctgagctgtgctgccagcggctacacttacagcatgtactgcatgggctggttcagacaagcccccggcaaggaaagagagggcgtggcccagatcaatagcgatggcagcacaagctacgccgacagcgtgaagggaaggttcactatctccaaggacaacgccaagaacactctgtatctgcagatgaactctctgaagccagaggacactgccatgtactactgcgctgccgattctagggtgtacggcggcagctggtatgagaggctctgcggcccttacacatacgagtacaactactggggccaaggcacacaagtgactgtctcgagcggcggaggatcccaagtgcagctgcaagagagcggaggaggaagcgtccaagccggaggatctctgagactgagctgcgccgctagtggctactcctacagcagctactgcatgggctggtttaggcaagcccccggcaaggagagagaaggcgtggccactatcgacagcgacggcatgacaaggtacgccgacagcgtgaagggaaggttcacaatcagcaaggacaacgccaagaacacactgtatctgcagatgaactctctgaagccagaggacactgccatgtactactgtgccgctcctctgtacgactgtgatagcggcgctgtgggcagaaatccaccttattggggccaaggcactcaagtgacagtctcgtctgctagccaccatcaccatcaccac

SEQ ID NO: 374 - DNA, 795 bp, synthetic construct
  source 1..795: mol_type = other DNA; note = Description of Artificial Sequence: Synthetic polynucleotide
caagtgcagctgcaagagtccggaggaggcagcgtccaagccggaggctctctgaggctgagctgtgctgccagcggctacacttacagcatgtactgcatgggctggttcagacaagcccccggcaaggaaagagagggcgtggcccagatcaatagcgatggcagcacaagctacgccgacagcgtgaagggaaggttcactatctccaaggacaacgccaagaacactctgtatctgcagatgaactctctgaagccagaggacactgccatgtactactgcgctgccgattctagggtgtacggcggcagctggtatgagaggctctgcggcccttacacatacgagtacaactactggggccaaggcacacaagtgactgtctcgagcggcggaggatcccaagtgcagctgcaagagagcggaggcggcagcgtgcagactggaggctctctgagactgagctgtgctgccagcggctacacttatctgaggggctgtatgggctggtttaggcaagcccccggcaaggagagagagggcgtggccgtcatggatgtggtgggcgataggagaagctacatcgacagcgtgaagggaaggttcacaatctctagggacaatgccgccaacagcgtctatctgcagatggacaatctgaagccagaggacacagccatgtactactgcactgccggccctaactgtgtgggctggagaagcggactggattactggggccaaggcacacaagtgacagtctcgtctgctagccaccatcaccatcaccac

SEQ ID NO: 375 - DNA, 816 bp, synthetic construct
  source 1..816: mol_type = other DNA; note = Description of Artificial Sequence: Synthetic polynucleotide
caagtgcagctgcaagagagcggcggaggaagcgtgcaagccggaggatctctgagactgagctgcgctgtgagcggctacgcctactccacatactgcatgggctggtttaggcaagcccccggcaaagagagagagggcgtggctgctatcgatagcggcggcagcacaagctacgccgatagcgtgaagggaaggttcacaatcagcaaggacaacgccaagaacacactgtatctgaggatgaactctctgaagccagaggacacagccatgtactactgtgctgctgtgcctcctcctccagatggcggcagctgtctgtttctgggaccagagatcaaggtcagcaaggccgattttaggtactggggccaaggcacacaagtgacagtctcgagcggcggaggatcccaagtgcagctgcaagagagcggcggaggaagcgtgcaagctggaggctctctgaggctgagctgtgctgccagcggctacacttatagcagcggctgtatgggctggttcagacaagcccccggcaaggaaagggaagccgtggccgccatcaattccgatggcagcacaagctacgccgacagcgtgaagggaaggttcacaatcagcaaggacaacgccaagaacacactctatctgcagatgaactctctgaagccagaggacacagccatgtactactgcgccgctgagccttactgtagcggcggctacccaagatggagcgtcgctgagttcggctactggggccaaggcacacaagtgactgtctcgtctgctagccaccatcaccatcaccac

SEQ ID NO: 376 - DNA, 810 bp, synthetic construct
  source 1..810: mol_type = other DNA; note = Description of Artificial Sequence: Synthetic polynucleotide
caagtgcagctgcaagagagcggcggaggaagcgtgcaagccggaggatctctgagactgagctgcgctgtgagcggctacgcctactccacatactgcatgggctggtttaggcaagcccccggcaaagagagagagggcgtggctgctatcgatagcggcggcagcacaagctacgccgatagcgtgaagggaaggttcacaatcagcaaggacaacgccaagaacacactgtatctgaggatgaactctctgaagccagaggacacagccatgtactactgtgctgctgtgcctcctcctccagatggcggcagctgtctgtttctgggaccagagatcaaggtcagcaaggccgattttaggtactggggccaaggcacacaagtgacagtctcgagcggcggaggatcccaagtgcagctgcaagagagcggaggaggcagcgtccaagccggaggctctctgaggctgagctgtgctgccagcggctacacttacagcagctactgcatgggctggttcagacaagcccccggcaaggagagagagggcgtggctcacatcgacagcgacggctccacaagctacgccgatagcgtgaagggaaggttcacaatctccaaggacaacgccaagaacactctgtacctccagatgaactctctgaagccagaggacactgccatgtactactgtgccgccgatccaattcccggccccggctactgcgatggcggccctaacaagtactggggccaaggcacacaagtgactgtctcgtctgctagccaccatcaccatcaccac

SEQ ID NO: 377 - DNA, 801 bp, synthetic construct
  source 1..801: mol_type = other DNA; note = Description of Artificial Sequence: Synthetic polynucleotide
caagtgcagctgcaagagagcggcggaggaagcgtgcaagccggaggatctctgagactgagctgcgctgtgagcggctacgcctactccacatactgcatgggctggtttaggcaagcccccggcaaagagagagagggcgtggctgctatcgatagcggcggcagcacaagctacgccgatagcgtgaagggaaggttcacaatcagcaaggacaacgccaagaacacactgtatctgaggatgaactctctgaagccagaggacacagccatgtactactgtgctgctgtgcctcctcctccagatggcggcagctgtctgtttctgggaccagagatcaaggtcagcaaggccgattttaggtactggggccaaggcacacaagtgacagtctcgagcggcggaggatcccaagtgcagctgcaagagtccggaggaggaagcgtgcaagccggcggatctctgagactgagctgtgccgcctctaggtacacttacaacagctactgcatgggctggttcagacaagcccccggcaaggaaagagagggcgtggccactatcgatagcgacggcatgactaggtacgctgatagcgtcaagggaaggttcacaatctccaaggacaatgctaagaacactctgtacctccagatgaactctctgaagccagaggacacagccatgtactactgcgctgccgatgccgactgcactatcgccgccatgactactaatcctctgggccaaggcacacaagtgactgtctcgtctgctagccaccatcaccatcaccac

SEQ ID NO: 378 - DNA, 813 bp, synthetic construct
  source 1..813: mol_type = other DNA; note = Description of Artificial Sequence: Synthetic polynucleotide
caagtgcagctgcaagagagcggcggaggaagcgtgcaagccggaggatctctgagactgagctgcgctgtgagcggctacgcctactccacatactgcatgggctggtttaggcaagcccccggcaaagagagagagggcgtggctgctatcgatagcggcggcagcacaagctacgccgatagcgtgaagggaaggttcacaatcagcaaggacaacgccaagaacacactgtatctgaggatgaactctctgaagccagaggacacagccatgtactactgtgctgctgtgcctcctcctccagatggcggcagctgtctgtttctgggaccagagatcaaggtcagcaaggccgattttaggtactggggccaaggcacacaagtgacagtctcgagcggcggaggatcccaagtgcagctgcaagagtccggcggaggcagcgtccaagccggaggatctctgaggctgagctgtacagtgagcagatacactgccagcgtgaactacatgggctggttcagacaagcccccggcaaagagagagagggcgtggccacaatcttcactggcgccggcacaacatactacgccaactccgtcaagggaaggttcacaatctctagggacaacgccaagaacactgcctatctgcagatgaactccctcaagccagaggacactgccatctactactgcgccgtggatttcagaggcggactgctgtataggccagcctacgagtacacttataggggccaaggcacacaagtgacagtctcgtctgctagccaccatcaccatcaccac

SEQ ID NO: 379 - DNA, 816 bp, synthetic construct
  source 1..816: mol_type = other DNA; note = Description of Artificial Sequence: Synthetic polynucleotide
caagtgcagctgcaagagagcggcggaggaagcgtgcaagccggaggatctctgagactgagctgcgctgtgagcggctacgcctactccacatactgcatgggctggtttaggcaagcccccggcaaagagagagagggcgtggctgctatcgatagcggcggcagcacaagctacgccgatagcgtgaagggaaggttcacaatcagcaaggacaacgccaagaacacactgtatctgaggatgaactctctgaagccagaggacacagccatgtactactgtgctgctgtgcctcctcctccagatggcggcagctgtctgtttctgggaccagagatcaaggtcagcaaggccgattttaggtactggggccaaggcacacaagtgacagtctcgagcggcggaggatcccaagtgcagctgcaagagagcggaggaggcagcgtcgaagctggaggatctctgaggctgagctgtgctgccagcggctacactcacagcagctactgtatgggctggttcagacaagcccccggcaaggagagggaaggcgtggctgccatcgacgtggatggcagcactacttacgccgacagcgtgaagggaaggttcactatcagcaaggacaacgccaagaacacactctatctgcagatgaacagcctcaagccagaggacactggcatgtactactgcgccgccgagttcgccgattgcagcagcaactactttctgcctcccggcgccgtcagatattggggccaaggcactcaagtgacagtctcgtctgctagccaccatcaccatcaccac

SEQ ID NO: 380 - DNA, 810 bp, synthetic construct
  source 1..810: mol_type = other DNA; note = Description of Artificial Sequence: Synthetic polynucleotide
caagtgcagctgcaagagagcggcggaggaagcgtgcaagccggaggatctctgagactgagctgcgctgtgagcggctacgcctactccacatactgcatgggctggtttaggcaagcccccggcaaagagagagagggcgtggctgctatcgatagcggcggcagcacaagctacgccgatagcgtgaagggaaggttcacaatcagcaaggacaacgccaagaacacactgtatctgaggatgaactctctgaagccagaggacacagccatgtactactgtgctgctgtgcctcctcctccagatggcggcagctgtctgtttctgggaccagagatcaaggtcagcaaggccgattttaggtactggggccaaggcacacaagtgacagtctcgagcggcggaggatcccaagtgcagctgcaagagagcggaggaggaagcgtccaagccggaggatctctgagactgagctgcgccgctagtggctactcctacagcagctactgcatgggctggtttaggcaagcccccggcaaggagagagaaggcgtggccactatcgacagcgacggcatgacaaggtacgccgacagcgtgaagggaaggttcacaatcagcaaggacaacgccaagaacacactgtatctgcagatgaactctctgaagccagaggacactgccatgtactactgtgccgctcctctgtacgactgtgatagcggcgctgtgggcagaaatccaccttattggggccaaggcactcaagtgacagtctcgtctgctagccaccatcaccatcaccac

SEQ ID NO: 381 - DNA, 804 bp, synthetic construct
  source 1..804: mol_type = other DNA; note = Description of Artificial Sequence: Synthetic polynucleotide
caagtgcagctgcaagagagcggcggaggaagcgtgcaagccggaggatctctgagactgagctgcgctgtgagcggctacgcctactccacatactgcatgggctggtttaggcaagcccccggcaaagagagagagggcgtggctgctatcgatagcggcggcagcacaagctacgccgatagcgtgaagggaaggttcacaatcagcaaggacaacgccaagaacacactgtatctgaggatgaactctctgaagccagaggacacagccatgtactactgtgctgctgtgcctcctcctccagatggcggcagctgtctgtttctgggaccagagatcaaggtcagcaaggccgattttaggtactggggccaaggcacacaagtgacagtctcgagcggcggaggatcccaagtgcagctgcaagagagcggaggcggcagcgtgcagactggaggctctctgagactgagctgtgctgccagcggctacacttatctgaggggctgtatgggctggtttaggcaagcccccggcaaggagagagagggcgtggccgtcatggatgtggtgggcgataggagaagctacatcgacagcgtgaagggaaggttcacaatctctagggacaatgccgccaacagcgtctatctgcagatggacaatctgaagccagaggacacagccatgtactactgcactgccggccctaactgtgtgggctggagaagcggactggattactggggccaaggcacacaagtgacagtctcgtctgctagccaccatcaccatcaccac

SEQ ID NO: 382 - DNA, 795 bp, synthetic construct
  source 1..795: mol_type = other DNA; note = Description of Artificial Sequence: Synthetic polynucleotide
caagtgcagctgcaagagagcggaggaggaagcgtgcaagccggaggctctctgaggctgagctgtacagtgtccggctacacttacagctccaattgcatgggctggtttaggcaagcccccggcaaggaaagagagggcgtggccactatctacactggcggcggcaacacatactacgccgatagcgtgaagggaaggttcactatcagccaagataacgccaagaacacagtgtatctgcagatgaacaatctgaagccagaggacactgccatgtactactgtgctgctgagccactgtctagggtgtacggcggcagctgcccaactcctacattcgactactggggccaaggcacacaagtgactgtctcgagcggcggaggatcccaagtgcagctgcaagagagcggcggaggaagcgtgcaagctggaggctctctgaggctgagctgtgctgccagcggctacacttatagcagcggctgtatgggctggttcagacaagcccccggcaaggaaagggaagccgtggccgccatcaattccgatggcagcacaagctacgccgacagcgtgaagggaaggttcacaatcagcaaggacaacgccaagaacacactctatctgcagatgaactctctgaagccagaggacacagccatgtactactgcgccgctgagccttactgtagcggcggctacccaagatggagcgtcgctgagttcggctactggggccaaggcacacaagtgactgtctcgtctgctagccaccatcaccatcaccac

SEQ ID NO: 383 - DNA, 789 bp, synthetic construct
  source 1..789: mol_type = other DNA; note = Description of Artificial Sequence: Synthetic polynucleotide
caagtgcagctgcaagagagcggaggaggaagcgtgcaagccggaggctctctgaggctgagctgtacagtgtccggctacacttacagctccaattgcatgggctggtttaggcaagcccccggcaaggaaagagagggcgtggccactatctacactggcggcggcaacacatactacgccgatagcgtgaagggaaggttcactatcagccaagataacgccaagaacacagtgtatctgcagatgaacaatctgaagccagaggacactgccatgtactactgtgctgctgagccactgtctagggtgtacggcggcagctgcccaactcctacattcgactactggggccaaggcacacaagtgactgtctcgagcggcggaggatcccaagtgcagctgcaagagagcggaggaggcagcgtccaagccggaggctctctgaggctgagctgtgctgccagcggctacacttacagcagctactgcatgggctggttcagacaagcccccggcaaggagagagagggcgtggctcacatcgacagcgacggctccacaagctacgccgatagcgtgaagggaaggttcacaatctccaaggacaacgccaagaacactctgtacctccagatgaactctctgaagccagaggacactgccatgtactactgtgccgccgatccaattcccggccccggctactgcgatggcggccctaacaagtactggggccaaggcacacaagtgactgtctcgtctgctagccaccatcaccatcaccac

SEQ ID NO: 384 - DNA, 780 bp, synthetic construct
  source 1..780: mol_type = other DNA; note = Description of Artificial Sequence: Synthetic polynucleotide
caagtgcagctgcaagagagcggaggaggaagcgtgcaagccggaggctctctgaggctgagctgtacagtgtccggctacacttacagctccaattgcatgggctggtttaggcaagcccccggcaaggaaagagagggcgtggccactatctacactggcggcggcaacacatactacgccgatagcgtgaagggaaggttcactatcagccaagataacgccaagaacacagtgtatctgcagatgaacaatctgaagccagaggacactgccatgtactactgtgctgctgagccactgtctagggtgtacggcggcagctgcccaactcctacattcgactactggggccaaggcacacaagtgactgtctcgagcggcggaggatcccaagtgcagctgcaagagtccggaggaggaagcgtgcaagccggcggatctctgagactgagctgtgccgcctctaggtacacttacaacagctactgcatgggctggttcagacaagcccccggcaaggaaagagagggcgtggccactatcgatagcgacggcatgactaggtacgctgatagcgtcaagggaaggttcacaatctccaaggacaatgctaagaacactctgtacctccagatgaactctctgaagccagaggacacagccatgtactactgcgctgccgatgccgactgcactatcgccgccatgactactaatcctctgggccaaggcacacaagtgactgtctcgtctgctagccaccatcaccatcaccac

SEQ ID NO: 385 - DNA, 792 bp, synthetic construct
  source 1..792: mol_type = other DNA; note = Description of Artificial Sequence: Synthetic polynucleotide
caagtgcagctgcaagagagcggaggaggaagcgtgcaagccggaggctctctgaggctgagctgtacagtgtccggctacacttacagctccaattgcatgggctggtttaggcaagcccccggcaaggaaagagagggcgtggccactatctacactggcggcggcaacacatactacgccgatagcgtgaagggaaggttcactatcagccaagataacgccaagaacacagtgtatctgcagatgaacaatctgaagccagaggacactgccatgtactactgtgctgctgagccactgtctagggtgtacggcggcagctgcccaactcctacattcgactactggggccaaggcacacaagtgactgtctcgagcggcggaggatcccaagtgcagctgcaagagtccggcggaggcagcgtccaagccggaggatctctgaggctgagctgtacagtgagcagatacactgccagcgtgaactacatgggctggttcagacaagcccccggcaaagagagagagggcgtggccacaatcttcactggcgccggcacaacatactacgccaactccgtcaagggaaggttcacaatctctagggacaacgccaagaacactgcctatctgcagatgaactccctcaagccagaggacactgccatctactactgcgccgtggatttcagaggcggactgctgtataggccagcctacgagtacacttataggggccaaggcacacaagtgacagtctcgtctgctagccaccatcaccatcaccac

SEQ ID NO: 386 - DNA, 795 bp, synthetic construct
  source 1..795: mol_type = other DNA; note = Description of Artificial Sequence: Synthetic polynucleotide
caagtgcagctgcaagagagcggaggaggaagcgtgcaagccggaggctctctgaggctgagctgtacagtgtccggctacacttacagctccaattgcatgggctggtttaggcaagcccccggcaaggaaagagagggcgtggccactatctacactggcggcggcaacacatactacgccgatagcgtgaagggaaggttcactatcagccaagataacgccaagaacacagtgtatctgcagatgaacaatctgaagccagaggacactgccatgtactactgtgctgctgagccactgtctagggtgtacggcggcagctgcccaactcctacattcgactactggggccaaggcacacaagtgactgtctcgagcggcggaggatcccaagtgcagctgcaagagagcggaggaggcagcgtcgaagctggaggatctctgaggctgagctgtgctgccagcggctacactcacagcagctactgtatgggctggttcagacaagcccccggcaaggagagggaaggcgtggctgccatcgacgtggatggcagcactacttacgccgacagcgtgaagggaaggttcactatcagcaaggacaacgccaagaacacactctatctgcagatgaacagcctcaagccagaggacactggcatgtactactgcgccgccgagttcgccgattgcagcagcaactactttctgcctcccggcgccgtcagatattggggccaaggcactcaagtgacagtctcgtctgctagccaccatcaccatcaccac

SEQ ID NO: 387 - DNA, 789 bp, synthetic construct
  source 1..789: mol_type = other DNA; note = Description of Artificial Sequence: Synthetic polynucleotide
caagtgcagctgcaagagagcggaggaggaagcgtgcaagccggaggctctctgaggctgagctgtacagtgtccggctacacttacagctccaattgcatgggctggtttaggcaagcccccggcaaggaaagagagggcgtggccactatctacactggcggcggcaacacatactacgccgatagcgtgaagggaaggttcactatcagccaagataacgccaagaacacagtgtatctgcagatgaacaatctgaagccagaggacactgccatgtactactgtgctgctgagccactgtctagggtgtacggcggcagctgcccaactcctacattcgactactggggccaaggcacacaagtgactgtctcgagcggcggaggatcccaagtgcagctgcaagagagcggaggaggaagcgtccaagccggaggatctctgagactgagctgcgccgctagtggctactcctacagcagctactgcatgggctggtttaggcaagcccccggcaaggagagagaaggcgtggccactatcgacagcgacggcatgacaaggtacgccgacagcgtgaagggaaggttcacaatcagcaaggacaacgccaagaacacactgtatctgcagatgaactctctgaagccagaggacactgccatgtactactgtgccgctcctctgtacgactgtgatagcggcgctgtgggcagaaatccaccttattggggccaaggcactcaagtgacagtctcgtctgctagccaccatcaccatcaccac

SEQ ID NO: 388 - DNA, 783 bp, synthetic construct
  source 1..783: mol_type = other DNA; note = Description of Artificial Sequence: Synthetic polynucleotide
caagtgcagctgcaagagagcggaggaggaagcgtgcaagccggaggctctctgaggctgagctgtacagtgtccggctacacttacagctccaattgcatgggctggtttaggcaagcccccggcaaggaaagagagggcgtggccactatctacactggcggcggcaacacatactacgccgatagcgtgaagggaaggttcactatcagccaagataacgccaagaacacagtgtatctgcagatgaacaatctgaagccagaggacactgccatgtactactgtgctgctgagccactgtctagggtgtacggcggcagctgcccaactcctacattcgactactggggccaaggcacacaagtgactgtctcgagcggcggaggatcccaagtgcagctgcaagagagcggaggcggcagcgtgcagactggaggctctctgagactgagctgtgctgccagcggctacacttatctgaggggctgtatgggctggtttaggcaagcccccggcaaggagagagagggcgtggccgtcatggatgtggtgggcgataggagaagctacatcgacagcgtgaagggaaggttcacaatctctagggacaatgccgccaacagcgtctatctgcagatggacaatctgaagccagaggacacagccatgtactactgcactgccggccctaactgtgtgggctggagaagcggactggattactggggccaaggcacacaagtgacagtctcgtctgctagccaccatcaccatcaccac

SEQ ID NO: 389 - DNA, 792 bp, synthetic construct
  source 1..792: mol_type = other DNA; note = Description of Artificial Sequence: Synthetic polynucleotide
caagtgcagctgcaagagagcggaggaggaagcgtccaagccggaggctctctgaggctgagctgtggagccagcggctacacttacagcagctactgtatgggctggtttaggcaagtgcccggcaaggagagagagggcgtggccgtgatcgattccgatggcagcacaagctacgctgacagcgtgaagggaaggttcacaatcagcaaggacaacggcaagaacacactctatctgcagatgaacagcctcaagccagaggacacagccatgtactactgcgccgctgatctgggccactataggcctccttgtggcgtgctgtatctgggcatggattactggggcaagggcacacaagtgacagtctcgagcggcggaggatcccaagtgcagctgcaagagagcggcggaggaagcgtgcaagctggaggctctctgaggctgagctgtgctgccagcggctacacttatagcagcggctgtatgggctggttcagacaagcccccggcaaggaaagggaagccgtggccgccatcaattccgatggcagcacaagctacgccgacagcgtgaagggaaggttcacaatcagcaaggacaacgccaagaacacactctatctgcagatgaactctctgaagccagaggacacagccatgtactactgcgccgctgagccttactgtagcggcggctacccaagatggagcgtcgctgagttcggctactggggccaaggcacacaagtgactgtctcgtctgctagccaccatcaccatcaccac

SEQ ID NO: 390 - DNA, 786 bp, synthetic construct
  source 1..786: mol_type = other DNA; note = Description of Artificial Sequence: Synthetic polynucleotide
caagtgcagctgcaagagagcggaggaggaagcgtccaagccggaggctctctgaggctgagctgtggagccagcggctacacttacagcagctactgtatgggctggtttaggcaagtgcccggcaaggagagagagggcgtggccgtgatcgattccgatggcagcacaagctacgctgacagcgtgaagggaaggttcacaatcagcaaggacaacggcaagaacacactctatctgcagatgaacagcctcaagccagaggacacagccatgtactactgcgccgctgatctgggccactataggcctccttgtggcgtgctgtatctgggcatggattactggggcaagggcacacaagtgacagtctcgagcggcggaggatcccaagtgcagctgcaagagagcggaggaggcagcgtccaagccggaggctctctgaggctgagctgtgctgccagcggctacacttacagcagctactgcatgggctggttcagacaagcccccggcaaggagagagagggcgtggctcacatcgacagcgacggctccacaagctacgccgatagcgtgaagggaaggttcacaatctccaaggacaacgccaagaacactctgtacctccagatgaactctctgaagccagaggacactgccatgtactactgtgccgccgatccaattcccggccccggctactgcgatggcggccctaacaagtactggggccaaggcacacaagtgactgtctcgtctgctagccaccatcaccatcaccac

SEQ ID NO: 391 - DNA, 777 bp, synthetic construct
  source 1..777: mol_type = other DNA; note = Description of Artificial Sequence: Synthetic polynucleotide
caagtgcagctgcaagagagcggaggaggaagcgtccaagccggaggctctctgaggctgagctgtggagccagcggctacacttacagcagctactgtatgggctggtttaggcaagtgcccggcaaggagagagagggcgtggccgtgatcgattccgatggcagcacaagctacgctgacagcgtgaagggaaggttcacaatcagcaaggacaacggcaagaacacactctatctgcagatgaacagcctcaagccagaggacacagccatgtactactgcgccgctgatctgggccactataggcctccttgtggcgtgctgtatctgggcatggattactggggcaagggcacacaagtgacagtctcgagcggcggaggatcccaagtgcagctgcaagagtccggaggaggaagcgtgcaagccggcggatctctgagactgagctgtgccgcctctaggtacacttacaacagctactgcatgggctggttcagacaagcccccggcaaggaaagagagggcgtggccactatcgatagcgacggcatgactaggtacgctgatagcgtcaagggaaggttcacaatctccaaggacaatgctaagaacactctgtacctccagatgaactctctgaagccagaggacacagccatgtactactgcgctgccgatgccgactgcactatcgccgccatgactactaatcctctgggccaaggcacacaagtgactgtctcgtctgctagccaccatcaccatcaccac

SEQ ID NO: 392 - DNA, 789 bp, synthetic construct
  source 1..789: mol_type = other DNA; note = Description of Artificial Sequence: Synthetic polynucleotide
caagtgcagctgcaagagagcggaggaggaagcgtccaagccggaggctctctgaggctgagctgtggagccagcggctacacttacagcagctactgtatgggctggtttaggcaagtgcccggcaaggagagagagggcgtggccgtgatcgattccgatggcagcacaagctacgctgacagcgtgaagggaaggttcacaatcagcaaggacaacggcaagaacacactctatctgcagatgaacagcctcaagccagaggacacagccatgtactactgcgccgctgatctgggccactataggcctccttgtggcgtgctgtatctgggcatggattactggggcaagggcacacaagtgacagtctcgagcggcggaggatcccaagtgcagctgcaagagtccggcggaggcagcgtccaagccggaggatctctgaggctgagctgtacagtgagcagatacactgccagcgtgaactacatgggctggttcagacaagcccccggcaaagagagagagggcgtggccacaatcttcactggcgccggcacaacatactacgccaactccgtcaagggaaggttcacaatctctagggacaacgccaagaacactgcctatctgcagatgaactccctcaagccagaggacactgccatctactactgcgccgtggatttcagaggcggactgctgtataggccagcctacgagtacacttataggggccaaggcacacaagtgacagtctcgtctgctagccaccatcaccatcaccac

SEQ ID NO: 393 - DNA, 792 bp, synthetic construct
  source 1..792: mol_type = other DNA; note = Description of Artificial Sequence: Synthetic polynucleotide
caagtgcagctgcaagagagcggaggaggaagcgtccaagccggaggctctctgaggctgagctgtggagccagcggctacacttacagcagctactgtatgggctggtttaggcaagtgcccggcaaggagagagagggcgtggccgtgatcgattccgatggcagcacaagctacgctgacagcgtgaagggaaggttcacaatcagcaaggacaacggcaagaacacactctatctgcagatgaacagcctcaagccagaggacacagccatgtactactgcgccgctgatctgggccactataggcctccttgtggcgtgctgtatctgggcatggattactggggcaagggcacacaagtgacagtctcgagcggcggaggatcccaagtgcagctgcaagagagcggaggaggcagcgtcgaagctggaggatctctgaggctgagctgtgctgccagcggctacactcacagcagctactgtatgggctggttcagacaagcccccggcaaggagagggaaggcgtggctgccatcgacgtggatggcagcactacttacgccgacagcgtgaagggaaggttcactatcagcaaggacaacgccaagaacacactctatctgcagatgaacagcctcaagccagaggacactggcatgtactactgcgccgccgagttcgccgattgcagcagcaactactttctgcctcccggcgccgtcagatattggggccaaggcactcaagtgacagtctcgtctgctagccaccatcaccatcaccac

SEQ ID NO: 394 - DNA, 786 bp, synthetic construct
  source 1..786: mol_type = other DNA; note = Description of Artificial Sequence: Synthetic polynucleotide
caagtgcagctgcaagagagcggaggaggaagcgtccaagccggaggctctctgaggctgagctgtggagccagcggctacacttacagcagctactgtatgggctggtttaggcaagtgcccggcaaggagagagagggcgtggccgtgatcgattccgatggcagcacaagctacgctgacagcgtgaagggaaggttcacaatcagcaaggacaacggcaagaacacactctatctgcagatgaacagcctcaagccagaggacacagccatgtactactgcgccgctgatctgggccactataggcctccttgtggcgtgctgtatctgggcatggattactggggcaagggcacacaagtgacagtctcgagcggcggaggatcccaagtgcagctgcaagagagcggaggaggaagcgtccaagccggaggatctctgagactgagctgcgccgctagtggctactcctacagcagctactgcatgggctggtttaggcaagcccccggcaaggagagagaaggcgtggccactatcgacagcgacggcatgacaaggtacgccgacagcgtgaagggaaggttcacaatcagcaaggacaacgccaagaacacactgtatctgcagatgaactctctgaagccagaggacactgccatgtactactgtgccgctcctctgtacgactgtgatagcggcgctgtgggcagaaatccaccttattggggccaaggcactcaagtgacagtctcgtctgctagccaccatcaccatcaccac

SEQ ID NO: 395 - DNA, 780 bp, synthetic construct
  source 1..780: mol_type = other DNA; note = Description of Artificial Sequence: Synthetic polynucleotide
caagtgcagctgcaagagagcggaggaggaagcgtccaagccggaggctctctgaggctgagctgtggagccagcggctacacttacagcagctactgtatgggctggtttaggcaagtgcccggcaaggagagagagggcgtggccgtgatcgattccgatggcagcacaagctacgctgacagcgtgaagggaaggttcacaatcagcaaggacaacggcaagaacacactctatctgcagatgaacagcctcaagccagaggacacagccatgtactactgcgccgctgatctgggccactataggcctccttgtggcgtgctgtatctgggcatggattactggggcaagggcacacaagtgacagtctcgagcggcggaggatcccaagtgcagctgcaagagagcggaggcggcagcgtgcagactggaggctctctgagactgagctgtgctgccagcggctacacttatctgaggggctgtatgggctggtttaggcaagcccccggcaaggagagagagggcgtggccgtcatggatgtggtgggcgataggagaagctacatcgacagcgtgaagggaaggttcacaatctctagggacaatgccgccaacagcgtctatctgcagatggacaatctgaagccagaggacacagccatgtactactgcactgccggccctaactgtgtgggctggagaagcggactggattactggggccaaggcacacaagtgacagtctcgtctgctagccaccatcaccatcaccac

SEQ ID NO: 396 - DNA, 795 bp, synthetic construct
  source 1..795: mol_type = other DNA; note = Description of Artificial Sequence: Synthetic polynucleotide
caagtgcagctgcaagagagcggaggaggaagcgtccaagccggaggctctctgagactgagctgtgccgccagcggctactccaactgcagctacgacatgacttggtataggcaagcccccggcaaggagagggagttcgtgtccgccatccacagcgacggcagcactagatacgccgacagcgtgaagggaaggttcttcatcagccaagataacgccaagaacacagtgtatctgcagatgaactccctcaagccagaggacactgccatgtactactgcaagacagacccactgcactgcagagcccatggcggcagctggtatagcgtgagggccaactactggggccaaggcacacaagtgacagtctcgagcggcggaggatcccaagtgcagctgcaagagagcggcggaggaagcgtgcaagctggaggctctctgaggctgagctgtgctgccagcggctacacttatagcagcggctgtatgggctggttcagacaagcccccggcaaggaaagggaagccgtggccgccatcaattccgatggcagcacaagctacgccgacagcgtgaagggaaggttcacaatcagcaaggacaacgccaagaacacactctatctgcagatgaactctctgaagccagaggacacagccatgtactactgcgccgctgagccttactgtagcggcggctacccaagatggagcgtcgctgagttcggctactggggccaaggcacacaagtgactgtctcgtctgctagccaccatcaccatcaccac

SEQ ID NO: 397 - DNA, 789 bp, synthetic construct
  source 1..789: mol_type = other DNA; note = Description of Artificial Sequence: Synthetic polynucleotide
caagtgcagctgcaagagagcggaggaggaagcgtccaagccggaggctctctgagactgagctgtgccgccagcggctactccaactgcagctacgacatgacttggtataggcaagcccccggcaaggagagggagttcgtgtccgccatccacagcgacggcagcactagatacgccgacagcgtgaagggaaggttcttcatcagccaagataacgccaagaacacagtgtatctgcagatgaactccctcaagccagaggacactgccatgtactactgcaagacagacccactgcactgcagagcccatggcggcagctggtatagcgtgagggccaactactggggccaaggcacacaagtgacagtctcgagcggcggaggatcccaagtgcagctgcaagagagcggaggaggcagcgtccaagccggaggctctctgaggctgagctgtgctgccagcggctacacttacagcagctactgcatgggctggttcagacaagcccccggcaaggagagagagggcgtggctcacatcgacagcgacggctccacaagctacgccgatagcgtgaagggaaggttcacaatctccaaggacaacgccaagaacactctgtacctccagatgaactctctgaagccagaggacactgccatgtactactgtgccgccgatccaattcccggccccggctactgcgatggcggccctaacaagtactggggccaaggcacacaagtgactgtctcgtctgctagccaccatcaccatcaccac

SEQ ID NO: 398 - DNA, 780 bp, synthetic construct
  source 1..780: mol_type = other DNA; note = Description of Artificial Sequence: Synthetic polynucleotide
caagtgcagctgcaagagagcggaggaggaagcgtccaagccggaggctctctgagactgagctgtgccgccagcggctactccaactgcagctacgacatgacttggtataggcaagcccccggcaaggagagggagttcgtgtccgccatccacagcgacggcagcactagatacgccgacagcgtgaagggaaggttcttcatcagccaagataacgccaagaacacagtgtatctgcagatgaactccctcaagccagaggacactgccatgtactactgcaagacagacccactgcactgcagagcccatggcggcagctggtatagcgtgagggccaactactggggccaaggcacacaagtgacagtctcgagcggcggaggatcccaagtgcagctgcaagagtccggaggaggaagcgtgcaagccggcggatctctgagactgagctgtgccgcctctaggtacacttacaacagctactgcatgggctggttcagacaagcccccggcaaggaaagagagggcgtggccactatcgatagcgacggcatgactaggtacgctgatagcgtcaagggaaggttcacaatctccaaggacaatgctaagaacactctgtacctccagatgaactctctgaagccagaggacacagccatgtactactgcgctgccgatgccgactgcactatcgccgccatgactactaatcctctgggccaaggcacacaagtgactgtctcgtctgctagccaccatcaccatcaccac

SEQ ID NO: 399 - DNA, 792 bp, synthetic construct
  source 1..792: mol_type = other DNA; note = Description of Artificial Sequence: Synthetic polynucleotide
caagtgcagctgcaagagagcggaggaggaagcgtccaagccggaggctctctgagactgagctgtgccgccagcggctactccaactgcagctacgacatgacttggtataggcaagcccccggcaaggagagggagttcgtgtccgccatccacagcgacggcagcactagatacgccgacagcgtgaagggaaggttcttcatcagccaagataacgccaagaacacagtgtatctgcagatgaactccctcaagccagaggacactgccatgtactactgcaagacagacccactgcactgcagagcccatggcggcagctggtatagcgtgagggccaactactggggccaaggcacacaagtgacagtctcgagcggcggaggatcccaagtgcagctgcaagagtccggcggaggcagcgtccaagccggaggatctctgaggctgagctgtacagtgagcagatacactgccagcgtgaactacatgggctggttcagacaagcccccggcaaagagagagagggcgtggccacaatcttcactggcgccggcacaacatactacgccaactccgtcaagggaaggttcacaatctctagggacaacgccaagaacactgcctatctgcagatgaactccctcaagccagaggacactgccatctactactgcgccgtggatttcagaggcggactgctgtataggccagcctacgagtacacttataggggccaaggcacacaagtgacagtctcgtctgctagccaccatcaccatcaccac

SEQ ID NO: 400 - DNA, 795 bp, synthetic construct
  source 1..795: mol_type = other DNA; note = Description of Artificial Sequence: Synthetic polynucleotide
caagtgcagctgcaagagagcggaggaggaagcgtccaagccggaggctctctgagactgagctgtgccgccagcggctactccaactgcagctacgacatgacttggtataggcaagcccccggcaaggagagggagttcgtgtccgccatccacagcgacggcagcactagatacgccgacagcgtgaagggaaggttcttcatcagccaagataacgccaagaacacagtgtatctgcagatgaactccctcaagccagaggacactgccatgtactactgcaagacagacccactgcactgcagagcccatggcggcagctggtatagcgtgagggccaactactggggccaaggcacacaagtgacagtctcgagcggcggaggatcccaagtgcagctgcaagagagcggaggaggcagcgtcgaagctggaggatctctgaggctgagctgtgctgccagcggctacactcacagcagctactgtatgggctggttcagacaagcccccggcaaggagagggaaggcgtggctgccatcgacgtggatggcagcactacttacgccgacagcgtgaagggaaggttcactatcagcaaggacaacgccaagaacacactctatctgcagatgaacagcctcaagccagaggacactggcatgtactactgcgccgccgagttcgccgattgcagcagcaactactttctgcctcccggcgccgtcagatattggggccaaggcactcaagtgacagtctcgtctgctagccaccatcaccatcaccac

SEQ ID NO: 401 - DNA, 789 bp, synthetic construct
  source 1..789: mol_type = other DNA; note = Description of Artificial Sequence: Synthetic polynucleotide
caagtgcagctgcaagagagcggaggaggaagcgtccaagccggaggctctctgagactgagctgtgccgccagcggctactccaactgcagctacgacatgacttggtataggcaagcccccggcaaggagagggagttcgtgtccgccatccacagcgacggcagcactagatacgccgacagcgtgaagggaaggttcttcatcagccaagataacgccaagaacacagtgtatctgcagatgaactccctcaagccagaggacactgccatgtactactgcaagacagacccactgcactgcagagcccatggcggcagctggtatagcgtgagggccaactactggggccaaggcacacaagtgacagtctcgagcggcggaggatcccaagtgcagctgcaagagagcggaggaggaagcgtccaagccggaggatctctgagactgagctgcgccgctagtggctactcctacagcagctactgcatgggctggtttaggcaagcccccggcaaggagagagaaggcgtggccactatcgacagcgacggcatgacaaggtacgccgacagcgtgaagggaaggttcacaatcagcaaggacaacgccaagaacacactgtatctgcagatgaactctctgaagccagaggacactgccatgtactactgtgccgctcctctgtacgactgtgatagcggcgctgtgggcagaaatccaccttattggggccaaggcactcaagtgacagtctcgtctgctagccaccatcaccatcaccac

SEQ ID NO: 402 - DNA, 783 bp, synthetic construct
  source 1..783: mol_type = other DNA; note = Description of Artificial Sequence: Synthetic polynucleotide
caagtgcagctgcaagagagcggaggaggaagcgtccaagccggaggctctctgagactgagctgtgccgccagcggctactccaactgcagctacgacatgacttggtataggcaagcccccggcaaggagagggagttcgtgtccgccatccacagcgacggcagcactagatacgccgacagcgtgaagggaaggttcttcatcagccaagataacgccaagaacacagtgtatctgcagatgaactccctcaagccagaggacactgccatgtactactgcaagacagacccactgcactgcagagcccatggcggcagctggtatagcgtgagggccaactactggggccaaggcacacaagtgacagtctcgagcggcggaggatcccaagtgcagctgcaagagagcggaggcggcagcgtgcagactggaggctctctgagactgagctgtgctgccagcggctacacttatctgaggggctgtatgggctggtttaggcaagcccccggcaaggagagagagggcgtggccgtcatggatgtggtgggcgataggagaagctacatcgacagcgtgaagggaaggttcacaatctctagggacaatgccgccaacagcgtctatctgcagatggacaatctgaagccagaggacacagccatgtactactgcactgccggccctaactgtgtgggctggagaagcggactggattactggggccaaggcacacaagtgacagtctcgtctgctagccaccatcaccatcaccac

SEQ ID NO: 403 - DNA, 789 bp, synthetic construct
  source 1..789: mol_type = other DNA; note = Description of Artificial Sequence: Synthetic polynucleotide
caagtgcagctgcaagagagcggcggaggaagcgtgcaagctggaggctctctgaggctgagctgtgctgccagcggctacacttatagcagcggctgtatgggctggttcagacaagcccccggcaaggaaagggaagccgtggccgccatcaattccgatggcagcacaagctacgccgacagcgtgaagggaaggttcacaatcagcaaggacaacgccaagaacacactctatctgcagatgaactctctgaagccagaggacacagccatgtactactgcgccgctgagccttactgtagcggcggctacccaagatggagcgtcgctgagttcggctactggggccaaggcacacaagtgactgtctcgagcggcggaggatcccaagtgcagctgcaagagagcggaggaggaagcgtgcaagccggaggctctctgagactgagctgtgccgcctctaggtatctgtacagcatcgactacatggcttggttcagacagagccccggcaaggagagggagccagtggctgtcatctacactgcctccggcgccacattctatccagatagcgtgaagggaaggttcactatcagccaagataacgccaagatgacagtgtatctgcagatgaactctctgaagagcgaggacactgccatgtactactgtgccgccgtgaggaagacagatagctacctcttcgacgcccagagcttcacatactggggccaaggcacacaagtgacagtctcgtctgctagccaccatcaccatcaccac

SEQ ID NO: 404 - DNA, 801 bp, synthetic construct
  source 1..801: mol_type = other DNA; note = Description of Artificial Sequence: Synthetic polynucleotide
caagtgcagctgcaagagagcggcggaggaagcgtgcaagctggaggctctctgaggctgagctgtgctgccagcggctacacttatagcagcggctgtatgggctggttcagacaagcccccggcaaggaaagggaagccgtggccgccatcaattccgatggcagcacaagctacgccgacagcgtgaagggaaggttcacaatcagcaaggacaacgccaagaacacactctatctgcagatgaactctctgaagccagaggacacagccatgtactactgcgccgctgagccttactgtagcggcggctacccaagatggagcgtcgctgagttcggctactggggccaaggcacacaagtgactgtctcgagcggcggaggatcccaagtgcagctgcaagagagcggaggaggaagcgtgcaagccggaggctctctgaggctgagctgtgccgcctctaggttcacatacagcagctactgcatgggctggttcagacaagcccccggcaaagagagagaaggcgtggccagcatcgatagcgatggctccactagctacactgacagcgtgaagggaaggttcactatcagcaaggacaacgccaagaacactctgtatctgcagatgaactctctgaagccagaggacacagccatgtactactgtgccctcgatctgatgagcacagtggtgcccggcttctgtggctttctgctgagcgctggcatggattactggggcaagggcactcaagtgactgtctcgtctgctagccaccatcaccatcaccac

SEQ ID NO: 405 - DNA, 807 bp, synthetic construct
  source 1..807: mol_type = other DNA; note = Description of Artificial Sequence: Synthetic polynucleotide
caagtgcagctgcaagagagcggcggaggaagcgtgcaagctggaggctctctgaggctgagctgtgctgccagcggctacacttatagcagcggctgtatgggctggttcagacaagcccccggcaaggaaagggaagccgtggccgccatcaattccgatggcagcacaagctacgccgacagcgtgaagggaaggttcacaatcagcaaggacaacgccaagaacacactctatctgcagatgaactctctgaagccagaggacacagccatgtactactgcgccgctgagccttactgtagcggcggctacccaagatggagcgtcgctgagttcggctactggggccaaggcacacaagtgactgtctcgagcggcggaggatcccaagtgcagctgcaagagtccggaggaggcagcgtccaagccggaggctctctgaggctgagctgtgctgccagcggctacacttacagcatgtactgcatgggctggttcagacaagcccccggcaaggaaagagagggcgtggcccagatcaatagcgatggcagcacaagctacgccgacagcgtgaagggaaggttcactatctccaaggacaacgccaagaacactctgtatctgcagatgaactctctgaagccagaggacactgccatgtactactgcgctgccgattctagggtgtacggcggcagctggtatgagaggctctgcggcccttacacatacgagtacaactactggggccaaggcacacaagtgactgtctcgtctgctagccaccatcaccatcaccac

SEQ ID NO: 406 - DNA, 816 bp, synthetic construct
  source 1..816: mol_type = other DNA; note = Description of Artificial Sequence: Synthetic polynucleotide
caagtgcagctgcaagagagcggcggaggaagcgtgcaagctggaggctctctgaggctgagctgtgctgccagcggctacacttatagcagcggctgtatgggctggttcagacaagcccccggcaaggaaagggaagccgtggccgccatcaattccgatggcagcacaagctacgccgacagcgtgaagggaaggttcacaatcagcaaggacaacgccaagaacacactctatctgcagatgaactctctgaagccagaggacacagccatgtactactgcgccgctgagccttactgtagcggcggctacccaagatggagcgtcgctgagttcggctactggggccaaggcacacaagtgactgtctcgagcggcggaggatcccaagtgcagctgcaagagagcggcggaggaagcgtgcaagccggaggatctctgagactgagctgcgctgtgagcggctacgcctactccacatactgcatgggctggtttaggcaagcccccggcaaagagagagagggcgtggctgctatcgatagcggcggcagcacaagctacgccgatagcgtgaagggaaggttcacaatcagcaaggacaacgccaagaacacactgtatctgaggatgaactctctgaagccagaggacacagccatgtactactgtgctgctgtgcctcctcctccagatggcggcagctgtctgtttctgggaccagagatcaaggtcagcaaggccgattttaggtactggggccaaggcacacaagtgacagtctcgtctgctagccaccatcaccatcaccac

SEQ ID NO: 407 - DNA, 795 bp, synthetic construct
  source 1..795: mol_type = other DNA; note = Description of Artificial Sequence: Synthetic polynucleotide
caagtgcagctgcaagagagcggcggaggaagcgtgcaagctggaggctctctgaggctgagctgtgctgccagcggctacacttatagcagcggctgtatgggctggttcagacaagcccccggcaaggaaagggaagccgtggccgccatcaattccgatggcagcacaagctacgccgacagcgtgaagggaaggttcacaatcagcaaggacaacgccaagaacacactctatctgcagatgaactctctgaagccagaggacacagccatgtactactgcgccgctgagccttactgtagcggcggctacccaagatggagcgtcgctgagttcggctactggggccaaggcacacaagtgactgtctcgagcggcggaggatcccaagtgcagctgcaagagagcggaggaggaagcgtgcaagccggaggctctctgaggctgagctgtacagtgtccggctacacttacagctccaattgcatgggctggtttaggcaagcccccggcaaggaaagagagggcgtggccactatctacactggcggcggcaacacatactacgccgatagcgtgaagggaaggttcactatcagccaagataacgccaagaacacagtgtatctgcagatgaacaatctgaagccagaggacactgccatgtactactgtgctgctgagccactgtctagggtgtacggcggcagctgcccaactcctacattcgactactggggccaaggcacacaagtgactgtctcgtctgctagccaccatcaccatcaccac

SEQ ID NO: 408 - DNA, 792 bp, synthetic construct
  source 1..792: mol_type = other DNA; note = Description of Artificial Sequence: Synthetic polynucleotide
caagtgcagctgcaagagagcggcggaggaagcgtgcaagctggaggctctctgaggctgagctgtgctgccagcggctacacttatagcagcggctgtatgggctggttcagacaagcccccggcaaggaaagggaagccgtggccgccatcaattccgatggcagcacaagctacgccgacagcgtgaagggaaggttcacaatcagcaaggacaacgccaagaacacactctatctgcagatgaactctctgaagccagaggacacagccatgtactactgcgccgctgagccttactgtagcggcggctacccaagatggagcgtcgctgagttcggctactggggccaaggcacacaagtgactgtctcgagcggcggaggatcccaagtgcagctgcaagagagcggaggaggaagcgtccaagccggaggctctctgaggctgagctgtggagccagcggctacacttacagcagctactgtatgggctggtttaggcaagtgcccggcaaggagagagagggcgtggccgtgatcgattccgatggcagcacaagctacgctgacagcgtgaagggaaggttcacaatcagcaaggacaacggcaagaacacactctatctgcagatgaacagcctcaagccagaggacacagccatgtactactgcgccgctgatctgggccactataggcctccttgtggcgtgctgtatctgggcatggattactggggcaagggcacacaagtgacagtctcgtctgctagccaccatcaccatcaccac

SEQ ID NO: 409 - DNA, 795 bp, synthetic construct
  source 1..795: mol_type = other DNA; note = Description of Artificial Sequence: Synthetic polynucleotide
caagtgcagctgcaagagagcggcggaggaagcgtgcaagctggaggctctctgaggctgagctgtgctgccagcggctacacttatagcagcggctgtatgggctggttcagacaagcccccggcaaggaaagggaagccgtggccgccatcaattccgatggcagcacaagctacgccgacagcgtgaagggaaggttcacaatcagcaaggacaacgccaagaacacactctatctgcagatgaactctctgaagccagaggacacagccatgtactactgcgccgctgagccttactgtagcggcggctacccaagatggagcgtcgctgagttcggctactggggccaaggcacacaagtgactgtctcgagcggcggaggatcccaagtgcagctgcaagagagcggaggaggaagcgtccaagccggaggctctctgagactgagctgtgccgccagcggctactccaactgcagctacgacatgacttggtataggcaagcccccggcaaggagagggagttcgtgtccgccatccacagcgacggcagcactagatacgccgacagcgtgaagggaaggttcttcatcagccaagataacgccaagaacacagtgtatctgcagatgaactccctcaagccagaggacactgccatgtactactgcaagacagacccactgcactgcagagcccatggcggcagctggtatagcgtgagggccaactactggggccaaggcacacaagtgacagtctcgtctgctagccaccatcaccatcaccac

SEQ ID NO: 410 - DNA, 783 bp, synthetic construct
  source 1..783: mol_type = other DNA; note = Description of Artificial Sequence: Synthetic polynucleotide
caagtgcagctgcaagagagcggaggaggcagcgtccaagccggaggctctctgaggctgagctgtgctgccagcggctacacttacagcagctactgcatgggctggttcagacaagcccccggcaaggagagagagggcgtggctcacatcgacagcgacggctccacaagctacgccgatagcgtgaagggaaggttcacaatctccaaggacaacgccaagaacactctgtacctccagatgaactctctgaagccagaggacactgccatgtactactgtgccgccgatccaattcccggccccggctactgcgatggcggccctaacaagtactggggccaaggcacacaagtgactgtctcgagcggcggaggatcccaagtgcagctgcaagagagcggaggaggaagcgtgcaagccggaggctctctgagactgagctgtgccgcctctaggtatctgtacagcatcgactacatggcttggttcagacagagccccggcaaggagagggagccagtggctgtcatctacactgcctccggcgccacattctatccagatagcgtgaagggaaggttcactatcagccaagataacgccaagatgacagtgtatctgcagatgaactctctgaagagcgaggacactgccatgtactactgtgccgccgtgaggaagacagatagctacctcttcgacgcccagagcttcacatactggggccaaggcacacaagtgacagtctcgtctgctagccaccatcaccatcaccac

SEQ ID NO: 411 - DNA, 795 bp, synthetic construct
  source 1..795: mol_type = other DNA; note = Description of Artificial Sequence: Synthetic polynucleotide
caagtgcagctgcaagagagcggaggaggcagcgtccaagccggaggctctctgaggctgagctgtgctgccagcggctacacttacagcagctactgcatgggctggttcagacaagcccccggcaaggagagagagggcgtggctcacatcgacagcgacggctccacaagctacgccgatagcgtgaagggaaggttcacaatctccaaggacaacgccaagaacactctgtacctccagatgaactctctgaagccagaggacactgccatgtactactgtgccgccgatccaattcccggccccggctactgcgatggcggccctaacaagtactggggccaaggcacacaagtgactgtctcgagcggcggaggatcccaagtgcagctgcaagagagcggaggaggaagcgtgcaagccggaggctctctgaggctgagctgtgccgcctctaggttcacatacagcagctactgcatgggctggttcagacaagcccccggcaaagagagagaaggcgtggccagcatcgatagcgatggctccactagctacactgacagcgtgaagggaaggttcactatcagcaaggacaacgccaagaacactctgtatctgcagatgaactctctgaagccagaggacacagccatgtactactgtgccctcgatctgatgagcacagtggtgcccggcttctgtggctttctgctgagcgctggcatggattactggggcaagggcactcaagtgactgtctcgtctgctagccaccatcaccatcaccac

SEQ ID NO: 412 - DNA, 801 bp, synthetic construct
  source 1..801: mol_type = other DNA; note = Description of Artificial Sequence: Synthetic polynucleotide
caagtgcagctgcaagagagcggaggaggcagcgtccaagccggaggctctctgaggctgagctgtgctgccagcggctacacttacagcagctactgcatgggctggttcagacaagcccccggcaaggagagagagggcgtggctcacatcgacagcgacggctccacaagctacgccgatagcgtgaagggaaggttcacaatctccaaggacaacgccaagaacactctgtacctccagatgaactctctgaagccagaggacactgccatgtactactgtgccgccgatccaattcccggccccggctactgcgatggcggccctaacaagtactggggccaaggcacacaagtgactgtctcgagcggcggaggatcccaagtgcagctgcaagagtccggaggaggcagcgtccaagccggaggctctctgaggctgagctgtgctgccagcggctacacttacagcatgtactgcatgggctggttcagacaagcccccggcaaggaaagagagggcgtggcccagatcaatagcgatggcagcacaagctacgccgacagcgtgaagggaaggttcactatctccaaggacaacgccaagaacactctgtatctgcagatgaactctctgaagccagaggacactgccatgtactactgcgctgccgattctagggtgtacggcggcagctggtatgagaggctctgcggcccttacacatacgagtacaactactggggccaaggcacacaagtgactgtctcgtctgctagccaccatcaccatcaccac

SEQ ID NO: 413 - DNA, 810 bp, synthetic construct
  source 1..810: mol_type = other DNA; note = Description of Artificial Sequence: Synthetic polynucleotide
caagtgcagctgcaagagagcggaggaggcagcgtccaagccggaggctctctgaggctgagctgtgctgccagcggctacacttacagcagctactgcatgggctggttcagacaagcccccggcaaggagagagagggcgtggctcacatcgacagcgacggctccacaagctacgccgatagcgtgaagggaaggttcacaatctccaaggacaacgccaagaacactctgtacctccagatgaactctctgaagccagaggacactgccatgtactactgtgccgccgatccaattcccggccccggctactgcgatggcggccctaacaagtactggggccaaggcacacaagtgactgtctcgagcggcggaggatcccaagtgcagctgcaagagagcggcggaggaagcgtgcaagccggaggatctctgagactgagctgcgctgtgagcggctacgcctactccacatactgcatgggctggtttaggcaagcccccggcaaagagagagagggcgtggctgctatcgatagcggcggcagcacaagctacgccgatagcgtgaagggaaggttcacaatcagcaaggacaacgccaagaacacactgtatctgaggatgaactctctgaagccagaggacacagccatgtactactgtgctgctgtgcctcctcctccagatggcggcagctgtctgtttctgggaccagagatcaaggtcagcaaggccgattttaggtactggggccaaggcacacaagtgacagtctcgtctgctagccaccatcaccatcaccac

SEQ ID NO: 414 - DNA, 789 bp, synthetic construct
  source 1..789: mol_type = other DNA; note = Description of Artificial Sequence: Synthetic polynucleotide
caagtgcagctgcaagagagcggaggaggcagcgtccaagccggaggctctctgaggctgagctgtgctgccagcggctacacttacagcagctactgcatgggctggttcagacaagcccccggcaaggagagagagggcgtggctcacatcgacagcgacggctccacaagctacgccgatagcgtgaagggaaggttcacaatctccaaggacaacgccaagaacactctgtacctccagatgaactctctgaagccagaggacactgccatgtactactgtgccgccgatccaattcccggccccggctactgcgatggcggccctaacaagtactggggccaaggcacacaagtgactgtctcgagcggcggaggatcccaagtgcagctgcaagagagcggaggaggaagcgtgcaagccggaggctctctgaggctgagctgtacagtgtccggctacacttacagctccaattgcatgggctggtttaggcaagcccccggcaaggaaagagagggcgtggccactatctacactggcggcggcaacacatactacgccgatagcgtgaagggaaggttcactatcagccaagataacgccaagaacacagtgtatctgcagatgaacaatctgaagccagaggacactgccatgtactactgtgctgctgagccactgtctagggtgtacggcggcagctgcccaactcctacattcgactactggggccaaggcacacaagtgactgtctcgtctgctagccaccatcaccatcaccac

SEQ ID NO: 415 - DNA, 786 bp, synthetic construct
  source 1..786: mol_type = other DNA; note = Description of Artificial Sequence: Synthetic polynucleotide
caagtgcagctgcaagagagcggaggaggcagcgtccaagccggaggctctctgaggctgagctgtgctgccagcggctacacttacagcagctactgcatgggctggttcagacaagcccccggcaaggagagagagggcgtggctcacatcgacagcgacggctccacaagctacgccgatagcgtgaagggaaggttcacaatctccaaggacaacgccaagaacactctgtacctccagatgaactctctgaagccagaggacactgccatgtactactgtgccgccgatccaattcccggccccggctactgcgatggcggccctaacaagtactggggccaaggcacacaagtgactgtctcgagcggcggaggatcccaagtgcagctgcaagagagcggaggaggaagcgtccaagccggaggctctctgaggctgagctgtggagccagcggctacacttacagcagctactgtatgggctggtttaggcaagtgcccggcaaggagagagagggcgtggccgtgatcgattccgatggcagcacaagctacgctgacagcgtgaagggaaggttcacaatcagcaaggacaacggcaagaacacactctatctgcagatgaacagcctcaagccagaggacacagccatgtactactgcgccgctgatctgggccactataggcctccttgtggcgtgctgtatctgggcatggattactggggcaagggcacacaagtgacagtctcgtctgctagccaccatcaccatcaccac

SEQ ID NO: 416 - DNA, 789 bp, synthetic construct
  source 1..789: mol_type = other DNA; note = Description of Artificial Sequence: Synthetic polynucleotide
caagtgcagctgcaagagagcggaggaggcagcgtccaagccggaggctctctgaggctgagctgtgctgccagcggctacacttacagcagctactgcatgggctggttcagacaagcccccggcaaggagagagagggcgtggctcacatcgacagcgacggctccacaagctacgccgatagcgtgaagggaaggttcacaatctccaaggacaacgccaagaacactctgtacctccagatgaactctctgaagccagaggacactgccatgtactactgtgccgccgatccaattcccggccccggctactgcgatggcggccctaacaagtactggggccaaggcacacaagtgactgtctcgagcggcggaggatcccaagtgcagctgcaagagagcggaggaggaagcgtccaagccggaggctctctgagactgagctgtgccgccagcggctactccaactgcagctacgacatgacttggtataggcaagcccccggcaaggagagggagttcgtgtccgccatccacagcgacggcagcactagatacgccgacagcgtgaagggaaggttcttcatcagccaagataacgccaagaacacagtgtatctgcagatgaactccctcaagccagaggacactgccatgtactactgcaagacagacccactgcactgcagagcccatggcggcagctggtatagcgtgagggccaactactggggccaaggcacacaagtgacagtctcgtctgctagccaccatcaccatcaccac

SEQ ID NO: 417 - DNA, 774 bp, synthetic construct
  source 1..774: mol_type = other DNA; note = Description of Artificial Sequence: Synthetic polynucleotide
caagtgcagctgcaagagtccggaggaggaagcgtgcaagccggcggatctctgagactgagctgtgccgcctctaggtacacttacaacagctactgcatgggctggttcagacaagcccccggcaaggaaagagagggcgtggccactatcgatagcgacggcatgactaggtacgctgatagcgtcaagggaaggttcacaatctccaaggacaatgctaagaacactctgtacctccagatgaactctctgaagccagaggacacagccatgtactactgcgctgccgatgccgactgcactatcgccgccatgactactaatcctctgggccaaggcacacaagtgactgtctcgagcggcggaggatcccaagtgcagctgcaagagagcggaggaggaagcgtgcaagccggaggctctctgagactgagctgtgccgcctctaggtatctgtacagcatcgactacatggcttggttcagacagagccccggcaaggagagggagccagtggctgtcatctacactgcctccggcgccacattctatccagatagcgtgaagggaaggttcactatcagccaagataacgccaagatgacagtgtatctgcagatgaactctctgaagagcgaggacactgccatgtactactgtgccgccgtgaggaagacagatagctacctcttcgacgcccagagcttcacatactggggccaaggcacacaagtgacagtctcgtctgctagccaccatcaccatcaccac

SEQ ID NO: 418 - DNA, 786 bp, synthetic construct
  source 1..786: mol_type = other DNA; note = Description of Artificial Sequence: Synthetic polynucleotide
caagtgcagctgcaagagtccggaggaggaagcgtgcaagccggcggatctctgagactgagctgtgccgcctctaggtacacttacaacagctactgcatgggctggttcagacaagcccccggcaaggaaagagagggcgtggccactatcgatagcgacggcatgactaggtacgctgatagcgtcaagggaaggttcacaatctccaaggacaatgctaagaacactctgtacctccagatgaactctctgaagccagaggacacagccatgtactactgcgctgccgatgccgactgcactatcgccgccatgactactaatcctctgggccaaggcacacaagtgactgtctcgagcggcggaggatcccaagtgcagctgcaagagagcggaggaggaagcgtgcaagccggaggctctctgaggctgagctgtgccgcctctaggttcacatacagcagctactgcatgggctggttcagacaagcccccggcaaagagagagaaggcgtggccagcatcgatagcgatggctccactagctacactgacagcgtgaagggaaggttcactatcagcaaggacaacgccaagaacactctgtatctgcagatgaactctctgaagccagaggacacagccatgtactactgtgccctcgatctgatgagcacagtggtgcccggcttctgtggctttctgctgagcgctggcatggattactggggcaagggcactcaagtgactgtctcgtctgctagccaccatcaccatcaccac

SEQ ID NO: 419 - DNA, 792 bp, synthetic construct
  source 1..792: mol_type = other DNA; note = Description of Artificial Sequence: Synthetic polynucleotide
caagtgcagctgcaagagtccggaggaggaagcgtgcaagccggcggatctctgagactgagctgtgccgcctctaggtacacttacaacagctactgcatgggctggttcagacaagcccccggcaaggaaagagagggcgtggccactatcgatagcgacggcatgactaggtacgctgatagcgtcaagggaaggttcacaatctccaaggacaatgctaagaacactctgtacctccagatgaactctctgaagccagaggacacagccatgtactactgcgctgccgatgccgactgcactatcgccgccatgactactaatcctctgggccaaggcacacaagtgactgtctcgagcggcggaggatcccaagtgcagctgcaagagtccggaggaggcagcgtccaagccggaggctctctgaggctgagctgtgctgccagcggctacacttacagcatgtactgcatgggctggttcagacaagcccccggcaaggaaagagagggcgtggcccagatcaatagcgatggcagcacaagctacgccgacagcgtgaagggaaggttcactatctccaaggacaacgccaagaacactctgtatctgcagatgaactctctgaagccagaggacactgccatgtactactgcgctgccgattctagggtgtacggcggcagctggtatgagaggctctgcggcccttacacatacgagtacaactactggggccaaggcacacaagtgactgtctcgtctgctagccaccatcaccatcaccac

SEQ ID NO: 420 - DNA, 801 bp, synthetic construct
  source 1..801: mol_type = other DNA; note = Description of Artificial Sequence: Synthetic polynucleotide
caagtgcagctgcaagagtccggaggaggaagcgtgcaagccggcggatctctgagactgagctgtgccgcctctaggtacacttacaacagctactgcatgggctggttcagacaagcccccggcaaggaaagagagggcgtggccactatcgatagcgacggcatgactaggtacgctgatagcgtcaagggaaggttcacaatctccaaggacaatgctaagaacactctgtacctccagatgaactctctgaagccagaggacacagccatgtactactgcgctgccgatgccgactgcactatcgccgccatgactactaatcctctgggccaaggcacacaagtgactgtctcgagcggcggaggatcccaagtgcagctgcaagagagcggcggaggaagcgtgcaagccggaggatctctgagactgagctgcgctgtgagcggctacgcctactccacatactgcatgggctggtttaggcaagcccccggcaaagagagagagggcgtggctgctatcgatagcggcggcagcacaagctacgccgatagcgtgaagggaaggttcacaatcagcaaggacaacgccaagaacacactgtatctgaggatgaactctctgaagccagaggacacagccatgtactactgtgctgctgtgcctcctcctccagatggcggcagctgtctgtttctgggaccagagatcaaggtcagcaaggccgattttaggtactggggccaaggcacacaagtgacagtctcgtctgctagccaccatcaccatcaccac

SEQ ID NO: 421 - DNA, 780 bp, synthetic construct
  source 1..780: mol_type = other DNA; note = Description of Artificial Sequence: Synthetic polynucleotide
caagtgcagctgcaagagtccggaggaggaagcgtgcaagccggcggatctctgagactgagctgtgccgcctctaggtacacttacaacagctactgcatgggctggttcagacaagcccccggcaaggaaagagagggcgtggccactatcgatagcgacggcatgactaggtacgctgatagcgtcaagggaaggttcacaatctccaaggacaatgctaagaacactctgtacctccagatgaactctctgaagccagaggacacagccatgtactactgcgctgccgatgccgactgcactatcgccgccatgactactaatcctctgggccaaggcacacaagtgactgtctcgagcggcggaggatcccaagtgcagctgcaagagagcggaggaggaagcgtgcaagccggaggctctctgaggctgagctgtacagtgtccggctacacttacagctccaattgcatgggctggtttaggcaagcccccggcaaggaaagagagggcgtggccactatctacactggcggcggcaacacatactacgccgatagcgtgaagggaaggttcactatcagccaagataacgccaagaacacagtgtatctgcagatgaacaatctgaagccagaggacactgccatgtactactgtgctgctgagccactgtctagggtgtacggcggcagctgcccaactcctacattcgactactggggccaaggcacacaagtgactgtctcgtctgctagccaccatcaccatcaccac

SEQ ID NO: 422 - DNA, 777 bp, synthetic construct
  source 1..777: mol_type = other DNA; note = Description of Artificial Sequence: Synthetic polynucleotide
caagtgcagctgcaagagtccggaggaggaagcgtgcaagccggcggatctctgagactgagctgtgccgcctctaggtacacttacaacagctactgcatgggctggttcagacaagcccccggcaaggaaagagagggcgtggccactatcgatagcgacggcatgactaggtacgctgatagcgtcaagggaaggttcacaatctccaaggacaatgctaagaacactctgtacctccagatgaactctctgaagccagaggacacagccatgtactactgcgctgccgatgccgactgcactatcgccgccatgactactaatcctctgggccaaggcacacaagtgactgtctcgagcggcggaggatcccaagtgcagctgcaagagagcggaggaggaagcgtccaagccggaggctctctgaggctgagctgtggagccagcggctacacttacagcagctactgtatgggctggtttaggcaagtgcccggcaaggagagagagggcgtggccgtgatcgattccgatggcagcacaagctacgctgacagcgtgaagggaaggttcacaatcagcaaggacaacggcaagaacacactctatctgcagatgaacagcctcaagccagaggacacagccatgtactactgcgccgctgatctgggccactataggcctccttgtggcgtgctgtatctgggcatggattactggggcaagggcacacaagtgacagtctcgtctgctagccaccatcaccatcaccac

SEQ ID NO: 423 - DNA, 780 bp, synthetic construct
  source 1..780: mol_type = other DNA; note = Description of Artificial Sequence: Synthetic polynucleotide
caagtgcagctgcaagagtccggaggaggaagcgtgcaagccggcggatctctgagactgagctgtgccgcctctaggtacacttacaacagctactgcatgggctggttcagacaagcccccggcaaggaaagagagggcgtggccactatcgatagcgacggcatgactaggtacgctgatagcgtcaagggaaggttcacaatctccaaggacaatgctaagaacactctgtacctccagatgaactctctgaagccagaggacacagccatgtactactgcgctgccgatgccgactgcactatcgccgccatgactactaatcctctgggccaaggcacacaagtgactgtctcgagcggcggaggatcccaagtgcagctgcaagagagcggaggaggaagcgtccaagccggaggctctctgagactgagctgtgccgccagcggctactccaactgcagctacgacatgacttggtataggcaagcccccggcaaggagagggagttcgtgtccgccatccacagcgacggcagcactagatacgccgacagcgtgaagggaaggttcttcatcagccaagataacgccaagaacacagtgtatctgcagatgaactccctcaagccagaggacactgccatgtactactgcaagacagacccactgcactgcagagcccatggcggcagctggtatagcgtgagggccaactactggggccaaggcacacaagtgacagtctcgtctgctagccaccatcaccatcaccac

SEQ ID NO: 424 - DNA, 786 bp, synthetic construct
  source 1..786: mol_type = other DNA; note = Description of Artificial Sequence: Synthetic polynucleotide
caagtgcagctgcaagagtccggcggaggcagcgtccaagccggaggatctctgaggctgagctgtacagtgagcagatacactgccagcgtgaactacatgggctggttcagacaagcccccggcaaagagagagagggcgtggccacaatcttcactggcgccggcacaacatactacgccaactccgtcaagggaaggttcacaatctctagggacaacgccaagaacactgcctatctgcagatgaactccctcaagccagaggacactgccatctactactgcgccgtggatttcagaggcggactgctgtataggccagcctacgagtacacttataggggccaaggcacacaagtgacagtctcgagcggcggaggatcccaagtgcagctgcaagagagcggaggaggaagcgtgcaagccggaggctctctgagactgagctgtgccgcctctaggtatctgtacagcatcgactacatggcttggttcagacagagccccggcaaggagagggagccagtggctgtcatctacactgcctccggcgccacattctatccagatagcgtgaagggaaggttcactatcagccaagataacgccaagatgacagtgtatctgcagatgaactctctgaagagcgaggacactgccatgtactactgtgccgccgtgaggaagacagatagctacctcttcgacgcccagagcttcacatactggggccaaggcacacaagtgacagtctcgtctgctagccaccatcaccatcaccac

SEQ ID NO: 425 - DNA, 798 bp, synthetic construct
  source 1..798: mol_type = other DNA; note = Description of Artificial Sequence: Synthetic polynucleotide
caagtgcagctgcaagagtccggcggaggcagcgtccaagccggaggatctctgaggctgagctgtacagtgagcagatacactgccagcgtgaactacatgggctggttcagacaagcccccggcaaagagagagagggcgtggccacaatcttcactggcgccggcacaacatactacgccaactccgtcaagggaaggttcacaatctctagggacaacgccaagaacactgcctatctgcagatgaactccctcaagccagaggacactgccatctactactgcgccgtggatttcagaggcggactgctgtataggccagcctacgagtacacttataggggccaaggcacacaagtgacagtctcgagcggcggaggatcccaagtgcagctgcaagagagcggaggaggaagcgtgcaagccggaggctctctgaggctgagctgtgccgcctctaggttcacatacagcagctactgcatgggctggttcagacaagcccccggcaaagagagagaaggcgtggccagcatcgatagcgatggctccactagctacactgacagcgtgaagggaaggttcactatcagcaaggacaacgccaagaacactctgtatctgcagatgaactctctgaagccagaggacacagccatgtactactgtgccctcgatctgatgagcacagtggtgcccggcttctgtggctttctgctgagcgctggcatggattactggggcaagggcactcaagtgactgtctcgtctgctagccaccatcaccatcaccac

SEQ ID NO: 426 - DNA, 804 bp, synthetic construct
  source 1..804: mol_type = other DNA; note = Description of Artificial Sequence: Synthetic polynucleotide
caagtgcagctgcaagagtccggcggaggcagcgtccaagccggaggatctctgaggctgagctgtacagtgagcagatacactgccagcgtgaactacatgggctggttcagacaagcccccggcaaagagagagagggcgtggccacaatcttcactggcgccggcacaacatactacgccaactccgtcaagggaaggttcacaatctctagggacaacgccaagaacactgcctatctgcagatgaactccctcaagccagaggacactgccatctactactgcgccgtggatttcagaggcggactgctgtataggccagcctacgagtacacttataggggccaaggcacacaagtgacagtctcgagcggcggaggatcccaagtgcagctgcaagagtccggaggaggcagcgtccaagccggaggctctctgaggctgagctgtgctgccagcggctacacttacagcatgtactgcatgggctggttcagacaagcccccggcaaggaaagagagggcgtggcccagatcaatagcgatggcagcacaagctacgccgacagcgtgaagggaaggttcactatctccaaggacaacgccaagaacactctgtatctgcagatgaactctctgaagccagaggacactgccatgtactactgcgctgccgattctagggtgtacggcggcagctggtatgagaggctctgcggcccttacacatacgagtacaactactggggccaaggcacacaagtgactgtctcgtctgctagccaccatcaccatcaccac

SEQ ID NO: 427 - DNA, 813 bp, synthetic construct
  source 1..813: mol_type = other DNA; note = Description of Artificial Sequence: Synthetic polynucleotide
caagtgcagctgcaagagtccggcggaggcagcgtccaagccggaggatctctgaggctgagctgtacagtgagcagatacactgccagcgtgaactacatgggctggttcagacaagcccccggcaaagagagagagggcgtggccacaatcttcactggcgccggcacaacatactacgccaactccgtcaagggaaggttcacaatctctagggacaacgccaagaacactgcctatctgcagatgaactccctcaagccagaggacactgccatctactactgcgccgtggatttcagaggcggactgctgtataggccagcctacgagtacacttataggggccaaggcacacaagtgacagtctcgagcggcggaggatcccaagtgcagctgcaagagagcggcggaggaagcgtgcaagccggaggatctctgagactgagctgcgctgtgagcggctacgcctactccacatactgcatgggctggtttaggcaagcccccggcaaagagagagagggcgtggctgctatcgatagcggcggcagcacaagctacgccgatagcgtgaagggaaggttcacaatcagcaaggacaacgccaagaacacactgtatctgaggatgaactctctgaagccagaggacacagccatgtactactgtgctgctgtgcctcctcctccagatggcggcagctgtctgtttctgggaccagagatcaaggtcagcaaggccgattttaggtactggggccaaggcacacaagtgacagtctcgtctgctagccaccatcaccatcaccac

SEQ ID NO: 428 - DNA, 792 bp, synthetic construct
  source 1..792: mol_type = other DNA; note = Description of Artificial Sequence: Synthetic polynucleotide
caagtgcagctgcaagagtccggcggaggcagcgtccaagccggaggatctctgaggctgagctgtacagtgagcagatacactgccagcgtgaactacatgggctggttcagacaagcccccggcaaagagagagagggcgtggccacaatcttcactggcgccggcacaacatactacgccaactccgtcaagggaaggttcacaatctctagggacaacgccaagaacactgcctatctgcagatgaactccctcaagccagaggacactgccatctactactgcgccgtggatttcagaggcggactgctgtataggccagcctacgagtacacttataggggccaaggcacacaagtgacagtctcgagcggcggaggatcccaagtgcagctgcaagagagcggaggaggaagcgtgcaagccggaggctctctgaggctgagctgtacagtgtccggctacacttacagctccaattgcatgggctggtttaggcaagcccccggcaaggaaagagagggcgtggccactatctacactggcggcggcaacacatactacgccgatagcgtgaagggaaggttcactatcagccaagataacgccaagaacacagtgtatctgcagatgaacaatctgaagccagaggacactgccatgtactactgtgctgctgagccactgtctagggtgtacggcggcagctgcccaactcctacattcgactactggggccaaggcacacaagtgactgtctcgtctgctagccaccatcaccatcaccac

SEQ ID NO: 429 - DNA, 789 bp, synthetic construct
  source 1..789: mol_type = other DNA; note = Description of Artificial Sequence: Synthetic polynucleotide
caagtgcagctgcaagagtccggcggaggcagcgtccaagccggaggatctctgaggctgagctgtacagtgagcagatacactgccagcgtgaactacatgggctggttcagacaagcccccggcaaagagagagagggcgtggccacaatcttcactggcgccggcacaacatactacgccaactccgtcaagggaaggttcacaatctctagggacaacgccaagaacactgcctatctgcagatgaactccctcaagccagaggacactgccatctactactgcgccgtggatttcagaggcggactgctgtataggccagcctacgagtacacttataggggccaaggcacacaagtgacagtctcgagcggcggaggatcccaagtgcagctgcaagagagcggaggaggaagcgtccaagccggaggctctctgaggctgagctgtggagccagcggctacacttacagcagctactgtatgggctggtttaggcaagtgcccggcaaggagagagagggcgtggccgtgatcgattccgatggcagcacaagctacgctgacagcgtgaagggaaggttcacaatcagcaaggacaacggcaagaacacactctatctgcagatgaacagcctcaagccagaggacacagccatgtactactgcgccgctgatctgggccactataggcctccttgtggcgtgctgtatctgggcatggattactggggcaagggcacacaagtgacagtctcgtctgctagccaccatcaccatcaccac

SEQ ID NO: 430 - DNA, 792 bp, synthetic construct
  source 1..792: mol_type = other DNA; note = Description of Artificial Sequence: Synthetic polynucleotide
caagtgcagctgcaagagtccggcggaggcagcgtccaagccggaggatctctgaggctgagctgtacagtgagcagatacactgccagcgtgaactacatgggctggttcagacaagcccccggcaaagagagagagggcgtggccacaatcttcactggcgccggcacaacatactacgccaactccgtcaagggaaggttcacaatctctagggacaacgccaagaacactgcctatctgcagatgaactccctcaagccagaggacactgccatctactactgcgccgtggatttcagaggcggactgctgtataggccagcctacgagtacacttataggggccaaggcacacaagtgacagtctcgagcggcggaggatcccaagtgcagctgcaagagagcggaggaggaagcgtccaagccggaggctctctgagactgagctgtgccgccagcggctactccaactgcagctacgacatgacttggtataggcaagcccccggcaaggagagggagttcgtgtccgccatccacagcgacggcagcactagatacgccgacagcgtgaagggaaggttcttcatcagccaagataacgccaagaacacagtgtatctgcagatgaactccctcaagccagaggacactgccatgtactactgcaagacagacccactgcactgcagagcccatggcggcagctggtatagcgtgagggccaactactggggccaaggcacacaagtgacagtctcgtctgctagccaccatcaccatcaccac

SEQ ID NO: 431 - DNA, 789 bp, synthetic construct
  source 1..789: mol_type = other DNA; note = Description of Artificial Sequence: Synthetic polynucleotide
caagtgcagctgcaagagagcggaggaggcagcgtcgaagctggaggatctctgaggctgagctgtgctgccagcggctacactcacagcagctactgtatgggctggttcagacaagcccccggcaaggagagggaaggcgtggctgccatcgacgtggatggcagcactacttacgccgacagcgtgaagggaaggttcactatcagcaaggacaacgccaagaacacactctatctgcagatgaacagcctcaagccagaggacactggcatgtactactgcgccgccgagttcgccgattgcagcagcaactactttctgcctcccggcgccgtcagatattggggccaaggcactcaagtgacagtctcgagcggcggaggatcccaagtgcagctgcaagagagcggaggaggaagcgtgcaagccggaggctctctgagactgagctgtgccgcctctaggtatctgtacagcatcgactacatggcttggttcagacagagccccggcaaggagagggagccagtggctgtcatctacactgcctccggcgccacattctatccagatagcgtgaagggaaggttcactatcagccaagataacgccaagatgacagtgtatctgcagatgaactctctgaagagcgaggacactgccatgtactactgtgccgccgtgaggaagacagatagctacctcttcgacgcccagagcttcacatactggggccaaggcacacaagtgacagtctcgtctgctagccaccatcaccatcaccac

SEQ ID NO: 432 - DNA, 801 bp, synthetic construct
  source 1..801: mol_type = other DNA; note = Description of Artificial Sequence: Synthetic polynucleotide
caagtgcagctgcaagagagcggaggaggcagcgtcgaagctggaggatctctgaggctgagctgtgctgccagcggctacactcacagcagctactgtatgggctggttcagacaagcccccggcaaggagagggaaggcgtggctgccatcgacgtggatggcagcactacttacgccgacagcgtgaagggaaggttcactatcagcaaggacaacgccaagaacacactctatctgcagatgaacagcctcaagccagaggacactggcatgtactactgcgccgccgagttcgccgattgcagcagcaactactttctgcctcccggcgccgtcagatattggggccaaggcactcaagtgacagtctcgagcggcggaggatcccaagtgcagctgcaagagagcggaggaggaagcgtgcaagccggaggctctctgaggctgagctgtgccgcctctaggttcacatacagcagctactgcatgggctggttcagacaagcccccggcaaagagagagaaggcgtggccagcatcgatagcgatggctccactagctacactgacagcgtgaagggaaggttcactatcagcaaggacaacgccaagaacactctgtatctgcagatgaactctctgaagccagaggacacagccatgtactactgtgccctcgatctgatgagcacagtggtgcccggcttctgtggctttctgctgagcgctggcatggattactggggcaagggcactcaagtgactgtctcgtctgctagccaccatcaccatcaccac

SEQ ID NO: 433 - DNA, 807 bp, synthetic construct
  source 1..807: mol_type = other DNA; note = Description of Artificial Sequence: Synthetic polynucleotide
caagtgcagctgcaagagagcggaggaggcagcgtcgaagctggaggatctctgaggctgagctgtgctgccagcggctacactcacagcagctactgtatgggctggttcagacaagcccccggcaaggagagggaaggcgtggctgccatcgacgtggatggcagcactacttacgccgacagcgtgaagggaaggttcactatcagcaaggacaacgccaagaacacactctatctgcagatgaacagcctcaagccagaggacactggcatgtactactgcgccgccgagttcgccgattgcagcagcaactactttctgcctcccggcgccgtcagatattggggccaaggcactcaagtgacagtctcgagcggcggaggatcccaagtgcagctgcaagagtccggaggaggcagcgtccaagccggaggctctctgaggctgagctgtgctgccagcggctacacttacagcatgtactgcatgggctggttcagacaagcccccggcaaggaaagagagggcgtggcccagatcaatagcgatggcagcacaagctacgccgacagcgtgaagggaaggttcactatctccaaggacaacgccaagaacactctgtatctgcagatgaactctctgaagccagaggacactgccatgtactactgcgctgccgattctagggtgtacggcggcagctggtatgagaggctctgcggcccttacacatacgagtacaactactggggccaaggcacacaagtgactgtctcgtctgctagccaccatcaccatcaccac

SEQ ID NO: 434 - DNA, 816 bp, synthetic construct
  source 1..816: mol_type = other DNA; note = Description of Artificial Sequence: Synthetic polynucleotide
caagtgcagctgcaagagagcggaggaggcagcgtcgaagctggaggatctctgaggctgagctgtgctgccagcggctacactcacagcagctactgtatgggctggttcagacaagcccccggcaaggagagggaaggcgtggctgccatcgacgtggatggcagcactacttacgccgacagcgtgaagggaaggttcactatcagcaaggacaacgccaagaacacactctatctgcagatgaacagcctcaagccagaggacactggcatgtactactgcgccgccgagttcgccgattgcagcagcaactactttctgcctcccggcgccgtcagatattggggccaaggcactcaagtgacagtctcgagcggcggaggatcccaagtgcagctgcaagagagcggcggaggaagcgtgcaagccggaggatctctgagactgagctgcgctgtgagcggctacgcctactccacatactgcatgggctggtttaggcaagcccccggcaaagagagagagggcgtggctgctatcgatagcggcggcagcacaagctacgccgatagcgtgaagggaaggttcacaatcagcaaggacaacgccaagaacacactgtatctgaggatgaactctctgaagccagaggacacagccatgtactactgtgctgctgtgcctcctcctccagatggcggcagctgtctgtttctgggaccagagatcaaggtcagcaaggccgattttaggtactggggccaaggcacacaagtgacagtctcgtctgctagccaccatcaccatcaccac

SEQ ID NO: 435 - DNA, 795 bp, synthetic construct
  source 1..795: mol_type = other DNA; note = Description of Artificial Sequence: Synthetic polynucleotide
caagtgcagctgcaagagagcggaggaggcagcgtcgaagctggaggatctctgaggctgagctgtgctgccagcggctacactcacagcagctactgtatgggctggttcagacaagcccccggcaaggagagggaaggcgtggctgccatcgacgtggatggcagcactacttacgccgacagcgtgaagggaaggttcactatcagcaaggacaacgccaagaacacactctatctgcagatgaacagcctcaagccagaggacactggcatgtactactgcgccgccgagttcgccgattgcagcagcaactactttctgcctcccggcgccgtcagatattggggccaaggcactcaagtgacagtctcgagcggcggaggatcccaagtgcagctgcaagagagcggaggaggaagcgtgcaagccggaggctctctgaggctgagctgtacagtgtccggctacacttacagctccaattgcatgggctggtttaggcaagcccccggcaaggaaagagagggcgtggccactatctacactggcggcggcaacacatactacgccgatagcgtgaagggaaggttcactatcagccaagataacgccaagaacacagtgtatctgcagatgaacaatctgaagccagaggacactgccatgtactactgtgctgctgagccactgtctagggtgtacggcggcagctgcccaactcctacattcgactactggggccaaggcacacaagtgactgtctcgtctgctagccaccatcaccatcaccac

SEQ ID NO: 436 - DNA, 792 bp, synthetic construct
  source 1..792: mol_type = other DNA; note = Description of Artificial Sequence: Synthetic polynucleotide
caagtgcagctgcaagagagcggaggaggcagcgtcgaagctggaggatctctgaggctgagctgtgctgccagcggctacactcacagcagctactgtatgggctggttcagacaagcccccggcaaggagagggaaggcgtggctgccatcgacgtggatggcagcactacttacgccgacagcgtgaagggaaggttcactatcagcaaggacaacgccaagaacacactctatctgcagatgaacagcctcaagccagaggacactggcatgtactactgcgccgccgagttcgccgattgcagcagcaactactttctgcctcccggcgccgtcagatattggggccaaggcactcaagtgacagtctcgagcggcggaggatcccaagtgcagctgcaagagagcggaggaggaagcgtccaagccggaggctctctgaggctgagctgtggagccagcggctacacttacagcagctactgtatgggctggtttaggcaagtgcccggcaaggagagagagggcgtggccgtgatcgattccgatggcagcacaagctacgctgacagcgtgaagggaaggttcacaatcagcaaggacaacggcaagaacacactctatctgcagatgaacagcctcaagccagaggacacagccatgtactactgcgccgctgatctgggccactataggcctccttgtggcgtgctgtatctgggcatggattactggggcaagggcacacaagtgacagtctcgtctgctagccaccatcaccatcaccac

SEQ ID NO: 437 - DNA, 795 bp, synthetic construct
  source 1..795: mol_type = other DNA; note = Description of Artificial Sequence: Synthetic polynucleotide
caagtgcagctgcaagagagcggaggaggcagcgtcgaagctggaggatctctgaggctgagctgtgctgccagcggctacactcacagcagctactgtatgggctggttcagacaagcccccggcaaggagagggaaggcgtggctgccatcgacgtggatggcagcactacttacgccgacagcgtgaagggaaggttcactatcagcaaggacaacgccaagaacacactctatctgcagatgaacagcctcaagccagaggacactggcatgtactactgcgccgccgagttcgccgattgcagcagcaactactttctgcctcccggcgccgtcagatattggggccaaggcactcaagtgacagtctcgagcggcggaggatcccaagtgcagctgcaagagagcggaggaggaagcgtccaagccggaggctctctgagactgagctgtgccgccagcggctactccaactgcagctacgacatgacttggtataggcaagcccccggcaaggagagggagttcgtgtccgccatccacagcgacggcagcactagatacgccgacagcgtgaagggaaggttcttcatcagccaagataacgccaagaacacagtgtatctgcagatgaactccctcaagccagaggacactgccatgtactactgcaagacagacccactgcactgcagagcccatggcggcagctggtatagcgtgagggccaactactggggccaaggcacacaagtgacagtctcgtctgctagccaccatcaccatcaccac

SEQ ID NO: 438 - DNA, 783 bp, synthetic construct
  source 1..783: mol_type = other DNA; note = Description of Artificial Sequence: Synthetic polynucleotide
caagtgcagctgcaagagagcggaggaggaagcgtccaagccggaggatctctgagactgagctgcgccgctagtggctactcctacagcagctactgcatgggctggtttaggcaagcccccggcaaggagagagaaggcgtggccactatcgacagcgacggcatgacaaggtacgccgacagcgtgaagggaaggttcacaatcagcaaggacaacgccaagaacacactgtatctgcagatgaactctctgaagccagaggacactgccatgtactactgtgccgctcctctgtacgactgtgatagcggcgctgtgggcagaaatccaccttattggggccaaggcactcaagtgacagtctcgagcggcggaggatcccaagtgcagctgcaagagagcggaggaggaagcgtgcaagccggaggctctctgagactgagctgtgccgcctctaggtatctgtacagcatcgactacatggcttggttcagacagagccccggcaaggagagggagccagtggctgtcatctacactgcctccggcgccacattctatccagatagcgtgaagggaaggttcactatcagccaagataacgccaagatgacagtgtatctgcagatgaactctctgaagagcgaggacactgccatgtactactgtgccgccgtgaggaagacagatagctacctcttcgacgcccagagcttcacatactggggccaaggcacacaagtgacagtctcgtctgctagccaccatcaccatcaccac

SEQ ID NO: 439 - DNA, 795 bp, synthetic construct
  source 1..795: mol_type = other DNA; note = Description of Artificial Sequence: Synthetic polynucleotide
caagtgcagctgcaagagagcggaggaggaagcgtccaagccggaggatctctgagactgagctgcgccgctagtggctactcctacagcagctactgcatgggctggtttaggcaagcccccggcaaggagagagaaggcgtggccactatcgacagcgacggcatgacaaggtacgccgacagcgtgaagggaaggttcacaatcagcaaggacaacgccaagaacacactgtatctgcagatgaactctctgaagccagaggacactgccatgtactactgtgccgctcctctgtacgactgtgatagcggcgctgtgggcagaaatccaccttattggggccaaggcactcaagtgacagtctcgagcggcggaggatcccaagtgcagctgcaagagagcggaggaggaagcgtgcaagccggaggctctctgaggctgagctgtgccgcctctaggttcacatacagcagctactgcatgggctggttcagacaagcccccggcaaagagagagaaggcgtggccagcatcgatagcgatggctccactagctacactgacagcgtgaagggaaggttcactatcagcaaggacaacgccaagaacactctgtatctgcagatgaactctctgaagccagaggacacagccatgtactactgtgccctcgatctgatgagcacagtggtgcccggcttctgtggctttctgctgagcgctggcatggattactggggcaagggcactcaagtgactgtctcgtctgctagccaccatcaccatcaccac

SEQ ID NO: 440 - DNA, 801 bp, synthetic construct
  source 1..801: mol_type = other DNA; note = Description of Artificial Sequence: Synthetic polynucleotide
caagtgcagctgcaagagagcggaggaggaagcgtccaagccggaggatctctgagactgagctgcgccgctagtggctactcctacagcagctactgcatgggctggtttaggcaagcccccggcaaggagagagaaggcgtggccactatcgacagcgacggcatgacaaggtacgccgacagcgtgaagggaaggttcacaatcagcaaggacaacgccaagaacacactgtatctgcagatgaactctctgaagccagaggacactgccatgtactactgtgccgctcctctgtacgactgtgatagcggcgctgtgggcagaaatccaccttattggggccaaggcactcaagtgacagtctcgagcggcggaggatcccaagtgcagctgcaagagtccggaggaggcagcgtccaagccggaggctctctgaggctgagctgtgctgccagcggctacacttacagcatgtactgcatgggctggttcagacaagcccccggcaaggaaagagagggcgtggcccagatcaatagcgatggcagcacaagctacgccgacagcgtgaagggaaggttcactatctccaaggacaacgccaagaacactctgtatctgcagatgaactctctgaagccagaggacactgccatgtactactgcgctgccgattctagggtgtacggcggcagctggtatgagaggctctgcggcccttacacatacgagtacaactactggggccaaggcacacaagtgactgtctcgtctgctagccaccatcaccatcaccac

SEQ ID NO: 441 - DNA, 810 bp, synthetic construct
  source 1..810: mol_type = other DNA; note = Description of Artificial Sequence: Synthetic polynucleotide
caagtgcagctgcaagagagcggaggaggaagcgtccaagccggaggatctctgagactgagctgcgccgctagtggctactcctacagcagctactgcatgggctggtttaggcaagcccccggcaaggagagagaaggcgtggccactatcgacagcgacggcatgacaaggtacgccgacagcgtgaagggaaggttcacaatcagcaaggacaacgccaagaacacactgtatctgcagatgaactctctgaagccagaggacactgccatgtactactgtgccgctcctctgtacgactgtgatagcggcgctgtgggcagaaatccaccttattggggccaaggcactcaagtgacagtctcgagcggcggaggatcccaagtgcagctgcaagagagcggcggaggaagcgtgcaagccggaggatctctgagactgagctgcgctgtgagcggctacgcctactccacatactgcatgggctggtttaggcaagcccccggcaaagagagagagggcgtggctgctatcgatagcggcggcagcacaagctacgccgatagcgtgaagggaaggttcacaatcagcaaggacaacgccaagaacacactgtatctgaggatgaactctctgaagccagaggacacagccatgtactactgtgctgctgtgcctcctcctccagatggcggcagctgtctgtttctgggaccagagatcaaggtcagcaaggccgattttaggtactggggccaaggcacacaagtgacagtctcgtctgctagccaccatcaccatcaccac

SEQ ID NO: 442 - DNA, 789 bp, synthetic construct
  source 1..789: mol_type = other DNA; note = Description of Artificial Sequence: Synthetic polynucleotide
caagtgcagctgcaagagagcggaggaggaagcgtccaagccggaggatctctgagactgagctgcgccgctagtggctactcctacagcagctactgcatgggctggtttaggcaagcccccggcaaggagagagaaggcgtggccactatcgacagcgacggcatgacaaggtacgccgacagcgtgaagggaaggttcacaatcagcaaggacaacgccaagaacacactgtatctgcagatgaactctctgaagccagaggacactgccatgtactactgtgccgctcctctgtacgactgtgatagcggcgctgtgggcagaaatccaccttattggggccaaggcactcaagtgacagtctcgagcggcggaggatcccaagtgcagctgcaagagagcggaggaggaagcgtgcaagccggaggctctctgaggctgagctgtacagtgtccggctacacttacagctccaattgcatgggctggtttaggcaagcccccggcaaggaaagagagggcgtggccactatctacactggcggcggcaacacatactacgccgatagcgtgaagggaaggttcactatcagccaagataacgccaagaacacagtgtatctgcagatgaacaatctgaagccagaggacactgccatgtactactgtgctgctgagccactgtctagggtgtacggcggcagctgcccaactcctacattcgactactggggccaaggcacacaagtgactgtctcgtctgctagccaccatcaccatcaccac

SEQ ID NO: 443 - DNA, 786 bp, synthetic construct
  source 1..786: mol_type = other DNA; note = Description of Artificial Sequence: Synthetic polynucleotide
caagtgcagctgcaagagagcggaggaggaagcgtccaagccggaggatctctgagactgagctgcgccgctagtggctactcctacagcagctactgcatgggctggtttaggcaagcccccggcaaggagagagaaggcgtggccactatcgacagcgacggcatgacaaggtacgccgacagcgtgaagggaaggttcacaatcagcaaggacaacgccaagaacacactgtatctgcagatgaactctctgaagccagaggacactgccatgtactactgtgccgctcctctgtacgactgtgatagcggcgctgtgggcagaaatccaccttattggggccaaggcactcaagtgacagtctcgagcggcggaggatcccaagtgcagctgcaagagagcggaggaggaagcgtccaagccggaggctctctgaggctgagctgtggagccagcggctacacttacagcagctactgtatgggctggtttaggcaagtgcccggcaaggagagagagggcgtggccgtgatcgattccgatggcagcacaagctacgctgacagcgtgaagggaaggttcacaatcagcaaggacaacggcaagaacacactctatctgcagatgaacagcctcaagccagaggacacagccatgtactactgcgccgctgatctgggccactataggcctccttgtggcgtgctgtatctgggcatggattactggggcaagggcacacaagtgacagtctcgtctgctagccaccatcaccatcaccac

SEQ ID NO: 444 - DNA, 789 bp, synthetic construct
  source 1..789: mol_type = other DNA; note = Description of Artificial Sequence: Synthetic polynucleotide
caagtgcagctgcaagagagcggaggaggaagcgtccaagccggaggatctctgagactgagctgcgccgctagtggctactcctacagcagctactgcatgggctggtttaggcaagcccccggcaaggagagagaaggcgtggccactatcgacagcgacggcatgacaaggtacgccgacagcgtgaagggaaggttcacaatcagcaaggacaacgccaagaacacactgtatctgcagatgaactctctgaagccagaggacactgccatgtactactgtgccgctcctctgtacgactgtgatagcggcgctgtgggcagaaatccaccttattggggccaaggcactcaagtgacagtctcgagcggcggaggatcccaagtgcagctgcaagagagcggaggaggaagcgtccaagccggaggctctctgagactgagctgtgccgccagcggctactccaactgcagctacgacatgacttggtataggcaagcccccggcaaggagagggagttcgtgtccgccatccacagcgacggcagcactagatacgccgacagcgtgaagggaaggttcttcatcagccaagataacgccaagaacacagtgtatctgcagatgaactccctcaagccagaggacactgccatgtactactgcaagacagacccactgcactgcagagcccatggcggcagctggtatagcgtgagggccaactactggggccaaggcacacaagtgacagtctcgtctgctagccaccatcaccatcaccac

SEQ ID NO: 445 - DNA, 777 bp, synthetic construct
  source 1..777: mol_type = other DNA; note = Description of Artificial Sequence: Synthetic polynucleotide
caagtgcagctgcaagagagcggaggcggcagcgtgcagactggaggctctctgagactgagctgtgctgccagcggctacacttatctgaggggctgtatgggctggtttaggcaagcccccggcaaggagagagagggcgtggccgtcatggatgtggtgggcgataggagaagctacatcgacagcgtgaagggaaggttcacaatctctagggacaatgccgccaacagcgtctatctgcagatggacaatctgaagccagaggacacagccatgtactactgcactgccggccctaactgtgtgggctggagaagcggactggattactggggccaaggcacacaagtgacagtctcgagcggcggaggatcccaagtgcagctgcaagagagcggaggaggaagcgtgcaagccggaggctctctgagactgagctgtgccgcctctaggtatctgtacagcatcgactacatggcttggttcagacagagccccggcaaggagagggagccagtggctgtcatctacactgcctccggcgccacattctatccagatagcgtgaagggaaggttcactatcagccaagataacgccaagatgacagtgtatctgcagatgaactctctgaagagcgaggacactgccatgtactactgtgccgccgtgaggaagacagatagctacctcttcgacgcccagagcttcacatactggggccaaggcacacaagtgacagtctcgtctgctagccaccatcaccatcaccac

SEQ ID NO: 446 - DNA, 789 bp, synthetic construct
  source 1..789: mol_type = other DNA; note = Description of Artificial Sequence: Synthetic polynucleotide
caagtgcagctgcaagagagcggaggcggcagcgtgcagactggaggctctctgagactgagctgtgctgccagcggctacacttatctgaggggctgtatgggctggtttaggcaagcccccggcaaggagagagagggcgtggccgtcatggatgtggtgggcgataggagaagctacatcgacagcgtgaagggaaggttcacaatctctagggacaatgccgccaacagcgtctatctgcagatggacaatctgaagccagaggacacagccatgtactactgcactgccggccctaactgtgtgggctggagaagcggactggattactggggccaaggcacacaagtgacagtctcgagcggcggaggatcccaagtgcagctgcaagagagcggaggaggaagcgtgcaagccggaggctctctgaggctgagctgtgccgcctctaggttcacatacagcagctactgcatgggctggttcagacaagcccccggcaaagagagagaaggcgtggccagcatcgatagcgatggctccactagctacactgacagcgtgaagggaaggttcactatcagcaaggacaacgccaagaacactctgtatctgcagatgaactctctgaagccagaggacacagccatgtactactgtgccctcgatctgatgagcacagtggtgcccggcttctgtggctttctgctgagcgctggcatggattactggggcaagggcactcaagtgactgtctcgtctgctagccaccatcaccatcaccac

SEQ ID NO: 447 - DNA, 795 bp, synthetic construct
  source 1..795: mol_type = other DNA; note = Description of Artificial Sequence: Synthetic polynucleotide
caagtgcagctgcaagagagcggaggcggcagcgtgcagactggaggctctctgagactgagctgtgctgccagcggctacacttatctgaggggctgtatgggctggtttaggcaagcccccggcaaggagagagagggcgtggccgtcatggatgtggtgggcgataggagaagctacatcgacagcgtgaagggaaggttcacaatctctagggacaatgccgccaacagcgtctatctgcagatggacaatctgaagccagaggacacagccatgtactactgcactgccggccctaactgtgtgggctggagaagcggactggattactggggccaaggcacacaagtgacagtctcgagcggcggaggatcccaagtgcagctgcaagagtccggaggaggcagcgtccaagccggaggctctctgaggctgagctgtgctgccagcggctacacttacagcatgtactgcatgggctggttcagacaagcccccggcaaggaaagagagggcgtggcccagatcaatagcgatggcagcacaagctacgccgacagcgtgaagggaaggttcactatctccaaggacaacgccaagaacactctgtatctgcagatgaactctctgaagccagaggacactgccatgtactactgcgctgccgattctagggtgtacggcggcagctggtatgagaggctctgcggcccttacacatacgagtacaactactggggccaaggcacacaagtgactgtctcgtctgctagccaccatcaccatcaccac

SEQ ID NO: 448 - DNA, 804 bp, synthetic construct
  source 1..804: mol_type = other DNA; note = Description of Artificial Sequence: Synthetic polynucleotide
caagtgcagctgcaagagagcggaggcggcagcgtgcagactggaggctctctgagactgagctgtgctgccagcggctacacttatctgaggggctgtatgggctggtttaggcaagcccccggcaaggagagagagggcgtggccgtcatggatgtggtgggcgataggagaagctacatcgacagcgtgaagggaaggttcacaatctctagggacaatgccgccaacagcgtctatctgcagatggacaatctgaagccagaggacacagccatgtactactgcactgccggccctaactgtgtgggctggagaagcggactggattactggggccaaggcacacaagtgacagtctcgagcggcggaggatcccaagtgcagctgcaagagagcggcggaggaagcgtgcaagccggaggatctctgagactgagctgcgctgtgagcggctacgcctactccacatactgcatgggctggtttaggcaagcccccggcaaagagagagagggcgtggctgctatcgatagcggcggcagcacaagctacgccgatagcgtgaagggaaggttcacaatcagcaaggacaacgccaagaacacactgtatctgaggatgaactctctgaagccagaggacacagccatgtactactgtgctgctgtgcctcctcctccagatggcggcagctgtctgtttctgggaccagagatcaaggtcagcaaggccgattttaggtactggggccaaggcacacaagtgacagtctcgtctgctagccaccatcaccatcaccac

SEQ ID NO: 449 - DNA, 783 bp, synthetic construct
  source 1..783: mol_type = other DNA; note = Description of Artificial Sequence: Synthetic polynucleotide
caagtgcagctgcaagagagcggaggcggcagcgtgcagactggaggctctctgagactgagctgtgctgccagcggctacacttatctgaggggctgtatgggctggtttaggcaagcccccggcaaggagagagagggcgtggccgtcatggatgtggtgggcgataggagaagctacatcgacagcgtgaagggaaggttcacaatctctagggacaatgccgccaacagcgtctatctgcagatggacaatctgaagccagaggacacagccatgtactactgcactgccggccctaactgtgtgggctggagaagcggactggattactggggccaaggcacacaagtgacagtctcgagcggcggaggatcccaagtgcagctgcaagagagcggaggaggaagcgtgcaagccggaggctctctgaggctgagctgtacagtgtccggctacacttacagctccaattgcatgggctggtttaggcaagcccccggcaaggaaagagagggcgtggccactatctacactggcggcggcaacacatactacgccgatagcgtgaagggaaggttcactatcagccaagataacgccaagaacacagtgtatctgcagatgaacaatctgaagccagaggacactgccatgtactactgtgctgctgagccactgtctagggtgtacggcggcagctgcccaactcctacattcgactactggggccaaggcacacaagtgactgtctcgtctgctagccaccatcaccatcaccac

SEQ ID NO: 450 - DNA, 780 bp, synthetic construct
  source 1..780: mol_type = other DNA; note = Description of Artificial Sequence: Synthetic polynucleotide
caagtgcagctgcaagagagcggaggcggcagcgtgcagactggaggctctctgagactgagctgtgctgccagcggctacacttatctgaggggctgtatgggctggtttaggcaagcccccggcaaggagagagagggcgtggccgtcatggatgtggtgggcgataggagaagctacatcgacagcgtgaagggaaggttcacaatctctagggacaatgccgccaacagcgtctatctgcagatggacaatctgaagccagaggacacagccatgtactactgcactgccggccctaactgtgtgggctggagaagcggactggattactggggccaaggcacacaagtgacagtctcgagcggcggaggatcccaagtgcagctgcaagagagcggaggaggaagcgtccaagccggaggctctctgaggctgagctgtggagccagcggctacacttacagcagctactgtatgggctggtttaggcaagtgcccggcaaggagagagagggcgtggccgtgatcgattccgatggcagcacaagctacgctgacagcgtgaagggaaggttcacaatcagcaaggacaacggcaagaacacactctatctgcagatgaacagcctcaagccagaggacacagccatgtactactgcgccgctgatctgggccactataggcctccttgtggcgtgctgtatctgggcatggattactggggcaagggcacacaagtgacagtctcgtctgctagccaccatcaccatcaccac

SEQ ID NO: 451 - DNA, 783 bp, synthetic construct
  source 1..783: mol_type = other DNA; note = Description of Artificial Sequence: Synthetic polynucleotide
caagtgcagctgcaagagagcggaggcggcagcgtgcagactggaggctctctgagactgagctgtgctgccagcggctacacttatctgaggggctgtatgggctggtttaggcaagcccccggcaaggagagagagggcgtggccgtcatggatgtggtgggcgataggagaagctacatcgacagcgtgaagggaaggttcacaatctctagggacaatgccgccaacagcgtctatctgcagatggacaatctgaagccagaggacacagccatgtactactgcactgccggccctaactgtgtgggctggagaagcggactggattactggggccaaggcacacaagtgacagtctcgagcggcggaggatcccaagtgcagctgcaagagagcggaggaggaagcgtccaagccggaggctctctgagactgagctgtgccgccagcggctactccaactgcagctacgacatgacttggtataggcaagcccccggcaaggagagggagttcgtgtccgccatccacagcgacggcagcactagatacgccgacagcgtgaagggaaggttcttcatcagccaagataacgccaagaacacagtgtatctgcagatgaactccctcaagccagaggacactgccatgtactactgcaagacagacccactgcactgcagagcccatggcggcagctggtatagcgtgagggccaactactggggccaaggcacacaagtgacagtctcgtctgctagccaccatcaccatcaccac

SEQ ID NO: 452 - protein, 578 aa, Homo sapiens
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 453 - protein, 214 aa, Homo sapiens
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 454 - protein, 575 aa, Mus musculus
MLSRLLPFLVTISSLSLEFIAYGTELPSPSYVWFEARFFQHILHWKPIPNQSESTYYEVALKQYGNSTWNDIHICRKAQALSCDLTTFTLDLYHRSYGYRARVRAVDNSQYSNWTTTETRFTVDEVILTVDSVTLKAMDGIIYGTIHPPRPTITPAGDEYEQVFKDLRVYKISIRKFSELKNATKRVKQETFTLTVPIGVRKFCVKVLPRLESRINKAEWSEEQCLLITTEQYFTVTNLSILVISMLLFCGILVCLVLQWYIRHPGKLPTVLVFKKPHDFFPANPLCPETPDAIHIVDLEVFPKVSLELRDSVLHGSTDSGFGSGKPSLQTEESQFLLPGSHPQIQGTLGKEESPGLQATCGDNTDSGICLQEPGLHSSMGPAWKQQLGYTHQDQDDSDVNLVQNSPGQPKYTQDASALGHVCLLEPKAPEEKDQVMVTFQGYQKQTRWKAEAAGPAECLDEEIPLTDAFDPELGVHLQDDLAWPPPALAAGYLKQESQGMASAPPGTPSRQWNQLTEEWSLLGVVSCEDLSIESWRFAHKLDPLDCGAAPGGLLDSLGSNLVTLPLISSLQVEE

SEQ ID NO: 455 - protein, 225 aa, Mus musculus
LEFIAYGTELPSPSYVWFEARFFQHILHWKPIPNQSESTYYEVALKQYGNSTWNDIHICRKAQALSCDLTTFTLDLYHRSYGYRARVRAVDNSQYSNWTTTETRFTVDEVILTVDSVTLKAMDGIIYGTIHPPRPTITPAGDEYEQVFKDLRVYKISIRKFSELKNATKRVKQETFTLTVPIGVRKFCVKVLPRLESRINKAEWSEEQCLLITTEQYFTVTNLSI

SEQ ID NO: 456 - protein, 325 aa, Homo sapiens
MAWSLGSWLGGCLLVSALGMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 457 - protein, 201 aa, Homo sapiens
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPS

SEQ ID NO: 458 - protein, 348 aa, Mus musculus
MAPCVAGWLGGFLLVPALGIPPPEKVRMNSVNFKNILQWEVPAFPKTNLTFTAQYESYRSFQDHCKRTASTQCDFSHLSKYGDYTVRVRAELADEHSEWVNVTFCPVEDTIIGPPEMQIESLAESLHLRFSAPQIENEPETWTLKNIYDSWAYRVQYWKNGTNEKFQVVSPYDSEVLRNLEPWTTYCIQVQGFLLDQNRTGEWSEPICERTGNDEITPSWIVAIILIVSVLVVFLFLLGCFVVLWLIYKKTKHTFRSGTSLPQHLKEFLGHPHHSTFLLFSFPPPEEAEVFDKLSIISEESEGSKQSPEDNCASEPPSDPGPRELESKDEAPSPPHDDPKLLTSTSEV

SEQ ID NO: 459 - protein, 201 aa, Mus musculus
MIPPPEKVRMNSVNFKNILQWEVPAFPKTNLTFTAQYESYRSFQDHCKRTASTQCDFSHLSKYGDYTVRVRAELADEHSEWVNVTFCPVEDTIIGPPEMQIESLAESLHLRFSAPQIENEPETWTLKNIYDSWAYRVQYWKNGTNEKFQVVSPYDSEVLRNLEPWTTYCIQVQGFLLDQNRTGEWSEPICERTGNDEITPS

SEQ ID NO: 460 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 461 - protein, 160 aa, Mus musculus
SRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 462 - protein, 5 aa, synthetic construct
  source 1..5: note = Description of Artificial Sequence: Synthetic peptide
GGGGS

SEQ ID NO: 463 - protein, 6 aa, synthetic construct
  source 1..6: note = Description of Artificial Sequence: Synthetic peptide
GGGGGS

SEQ ID NO: 464 - protein, 4 aa, synthetic construct
  source 1..4: note = Description of Artificial Sequence: Synthetic peptide
GGSG

SEQ ID NO: 465 - protein, 4 aa, synthetic construct
  source 1..4: note = Description of Artificial Sequence: Synthetic peptide
SGGG

SEQ ID NO: 466 - protein, 4 aa, synthetic construct
  source 1..4: note = Description of Artificial Sequence: Synthetic peptide
GSGS

SEQ ID NO: 467 - protein, 6 aa, synthetic construct
  source 1..6: note = Description of Artificial Sequence: Synthetic peptide
GSGSGS

SEQ ID NO: 468 - protein, 10 aa, synthetic construct
  source 1..10: note = Description of Artificial Sequence: Synthetic peptide
GSGSGSGSGS

SEQ ID NO: 469 - protein, 12 aa, synthetic construct
  source 1..12: note = Description of Artificial Sequence: Synthetic peptide
GSGSGSGSGSGS

SEQ ID NO: 470 - protein, 6 aa, synthetic construct
  source 1..6: note = Description of Artificial Sequence: Synthetic peptide
GGSGGS

SEQ ID NO: 471 - protein, 9 aa, synthetic construct
  source 1..9: note = Description of Artificial Sequence: Synthetic peptide
GGSGGSGGS

SEQ ID NO: 472 - protein, 10 aa, synthetic construct
  source 1..10: note = Description of Artificial Sequence: Synthetic peptide
GGSGGSGGSG

SEQ ID NO: 473 - protein, 8 aa, synthetic construct
  source 1..8: note = Description of Artificial Sequence: Synthetic peptide
GGSGGGSG

SEQ ID NO: 474 - protein, 12 aa, synthetic construct
  source 1..12: note = Description of Artificial Sequence: Synthetic peptide
GGSGGGSGGGSG

SEQ ID NO: 475 - protein, 16 aa, synthetic construct
  source 1..16: note = Description of Artificial Sequence: Synthetic peptide
GGSGGGSGGGSGGGSG

SEQ ID NO: 476 - protein, 20 aa, synthetic construct
  source 1..20: note = Description of Artificial Sequence: Synthetic peptide
GGSGGGSGGGSGGGSGGGSG

SEQ ID NO: 477 - protein, 10 aa, synthetic construct
  source 1..10: note = Description of Artificial Sequence: Synthetic peptide
GGGGSGGGGS

SEQ ID NO: 478 - protein, 8 aa, synthetic construct
  source 1..8: note = Description of Artificial Sequence: Synthetic peptide
GGGSGGGS

SEQ ID NO: 479 - protein, 5 aa, synthetic construct
  source 1..5: note = Description of Artificial Sequence: Synthetic peptide
GGSGG

SEQ ID NO: 480 - protein, 5 aa, synthetic construct
  source 1..5: note = Description of Artificial Sequence: Synthetic peptide
GSGSG

SEQ ID NO: 481 - protein, 5 aa, synthetic construct
  source 1..5: note = Description of Artificial Sequence: Synthetic peptide
GSGGG

SEQ ID NO: 482 - protein, 5 aa, synthetic construct
  source 1..5: note = Description of Artificial Sequence: Synthetic peptide
GGGSG

SEQ ID NO: 483 - protein, 5 aa, synthetic construct
  source 1..5: note = Description of Artificial Sequence: Synthetic peptide
GSSSG

SEQ ID NO: 484 - protein, 4 aa, synthetic construct
  source 1..4: note = Description of Artificial Sequence: Synthetic peptide
GGGS

SEQ ID NO: 485 - protein, 232 aa, synthetic construct
  source 1..232: note = Description of Artificial Sequence: Synthetic polypeptide
ASRVESKYGPPCPPCPAPEFLGGPSVFLFPPKPKDTLMISRTPEVTCVVVDVSQEDPEVQFNWYVDGVEVHNAKTKPREEQFGSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 486 - protein, 264 aa, synthetic construct
  source 1..264: note = Description of Artificial Sequence: Synthetic polypeptide
EVQLQESGGGSVQAGGSLRLSCAASGYSQCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSGGGSQVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSASHHHHHH

SEQ ID NO: 487 - protein, 265 aa, synthetic construct
  source 1..265: note = Description of Artificial Sequence: Synthetic polypeptide
EVQLQESGGGSVQAGGSLRLSCAASGYSDCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSQVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSASHHHHHH

SEQ ID NO: 488 - protein, 265 aa, synthetic construct
  source 1..265: note = Description of Artificial Sequence: Synthetic polypeptide
EVQLQESGGGSVQAGGSLRLSCAASGYSNCAYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSQVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSASHHHHHH

SEQ ID NO: 489 - protein, 256 aa, synthetic construct
  source 1..256: note = Description of Artificial Sequence: Synthetic polypeptide
EVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSQVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSASHHHHHH

SEQ ID NO: 490 - protein, 258 aa, synthetic construct
  source 1..258: note = Description of Artificial Sequence: Synthetic polypeptide
EVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGSQVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSASHHHHHH

SEQ ID NO: 491 - protein, 262 aa, synthetic construct
  source 1..262: note = Description of Artificial Sequence: Synthetic polypeptide
EVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGSGGSQVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSASHHHHHH

SEQ ID NO: 492 - protein, 264 aa, synthetic construct
  source 1..264: note = Description of Artificial Sequence: Synthetic polypeptide
EVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSGGGSQVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSASHHHHHH

SEQ ID NO: 493 - protein, 266 aa, synthetic construct
  source 1..266: note = Description of Artificial Sequence: Synthetic polypeptide
EVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGSGGSGGSGQVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSASHHHHHH

SEQ ID NO: 494 - protein, 261 aa, synthetic construct
  source 1..261: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGSQVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSASHHHHHH

SEQ ID NO: 495 - protein, 265 aa, synthetic construct
  source 1..265: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGSGGSQVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSASHHHHHH

SEQ ID NO: 496 - protein, 267 aa, synthetic construct
  source 1..267: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSGGGSQVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSASHHHHHH

SEQ ID NO: 497 - protein, 269 aa, synthetic construct
  source 1..269: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGSGGSGGSGQVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSASHHHHHH

SEQ ID NO: 498 - protein, 263 aa, synthetic construct
  source 1..263: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGSQVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSASHHHHHH

SEQ ID NO: 499 - protein, 484 aa, synthetic construct
  source 1..484: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSQVQLQESGGGSVQAGGSLRLSCAASRYTYNSYCMGWFRQAPGKEREGVATIDSDGMTRYADSVKGRFTISKDNAKNTLYLQMNSLKPEDTAMYYCAADADCTIAAMTTNPLGQGTQVTVSSASRVESKYGPPCPPCPAPEFLGGPSVFLFPPKPKDTLMISRTPEVTCVVVDVSQEDPEVQFNWYVDGVEVHNAKTKPREEQFGSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 500 - protein, 487 aa, synthetic construct
  source 1..487: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSQVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSASRVESKYGPPCPPCPAPEFLGGPSVFLFPPKPKDTLMISRTPEVTCVVVDVSQEDPEVQFNWYVDGVEVHNAKTKPREEQFGSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 501 - protein, 487 aa, synthetic construct
  source 1..487: note = Description of Artificial Sequence: Synthetic polypeptide
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGSQVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSSASRVESKYGPPCPPCPAPEFLGGPSVFLFPPKPKDTLMISRTPEVTCVVVDVSQEDPEVQFNWYVDGVEVHNAKTKPREEQFGSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 502 - protein, 230 aa, synthetic construct
  source 1..230: note = Description of Artificial Sequence: Synthetic polypeptide
RVESKYGPPCPPCPAPEFLGGPSVFLFPPKPKDTLMISRTPEVTCVVVDVSQEDPEVQFNWYVDGVEVHNAKTKPREEQFGSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 503 - protein, 127 aa, synthetic construct
  source 1..127: note = Description of Artificial Sequence: Synthetic polypeptide
EVQLLESGGGLVQPGGSLRLSCAASGYTQCSYDMTWVRQAPGKGLEWVSAIHSDGSTRYADSVKGRFTISRDNSKNTLYLQMNSLRAEDTAVYYCKTDPLHCRAHGGSWYSVRANYWGQGTLVTVSS

SEQ ID NO: 504 - protein, 127 aa, synthetic construct
  source 1..127: note = Description of Artificial Sequence: Synthetic polypeptide
EVQLLESGGGLVQPGGSLRLSCAASGYTQCSYDMTWVRQAPGKGLEFVSAIHSDGSTRYADSVKGRFTISRDNSKNTLYLQMNSLRAEDTAVYYCKTDPLHCRAHGGSWYSVRANYWGQGTLVTVSS

SEQ ID NO: 505 - protein, 127 aa, synthetic construct
  source 1..127: note = Description of Artificial Sequence: Synthetic polypeptide
EVQLVESGGGLVQPGGSLRLSCAASGYTQCSYDMTWVRQAPGKGLEWVSAIHSDGSTRYADSVKGRFFISRDNSKNTLYLQMNSLRAEDTAVYYCKTDPLHCRAHGGSWYSVRANYWGQGTLVTVSS

SEQ ID NO: 506 - protein, 127 aa, synthetic construct
  source 1..127: note = Description of Artificial Sequence: Synthetic polypeptide
EVQLVESGGGLVQPGGSLRLSCAASGYTQCSYDMTWVRQAPGKGLEFVSAIHSDGSTRYADSVKGRFFISRDNSKNTLYLQMNSLRAEDTAVYYCKTDPLHCRAHGGSWYSVRANYWGQGTLVTVSS

SEQ ID NO: 507 - protein, 126 aa, synthetic construct
  source 1..126: note = Description of Artificial Sequence: Synthetic polypeptide
EVQLLESGGGLVQPGGSLRLSCAASGYTHSSYCMGWVRQAPGKGLEWVSAIDVDGSTTYADSVKGRFTISRDNSKNTLYLQMNSLRAEDTAVYYCAAEFADCSSNYFLPPGAVRYWGQGTLVTVSS

SEQ ID NO: 508 - protein, 126 aa, synthetic construct
  source 1..126: note = Description of Artificial Sequence: Synthetic polypeptide
EVQLLESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 509 - protein, 126 aa, synthetic construct
  source 1..126: note = Description of Artificial Sequence: Synthetic polypeptide
EVQLVESGGGLVQPGGSLRLSCAASGYTHSSYCMGWVRQAPGKGLEWVSAIDVDGSTTYADSVKGRFTISRDNSKNTLYLQMNSLRAEDTAVYYCAAEFADCSSNYFLPPGAVRYWGQGTLVTVSS

SEQ ID NO: 510 - protein, 126 aa, synthetic construct
  source 1..126: note = Description of Artificial Sequence: Synthetic polypeptide
EVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 511 - protein, 127 aa, synthetic construct
  source 1..127: note = Description of Artificial Sequence: Synthetic polypeptide
EVQLVESGGGLVQPGGSLRLSCAASGYTQCSYDMTWVRQAPGKGREFVSAIHSDGSTRYADSVKGRFFISRDNSKNTLYLQMNSLRAEDTAVYYCKTDPLHCRAHGGSWYSVRANYWGQGTLVTVSS

SEQ ID NO: 512 - protein, 127 aa, synthetic construct
  source 1..127: note = Description of Artificial Sequence: Synthetic polypeptide
EVQLQESGGGSVQAGGSLRLSCAASGYSQCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSS

SEQ ID NO: 513 - protein, 265 aa, synthetic construct
  source 1..265: note = Description of Artificial Sequence: Synthetic polypeptide
EVQLVESGGGLVKPGGSLRLSCAASGYSNCAYDMTWYRQAPGKGLEWVSAIHSDGSTRYADSVKGRFFISRDNAKNSLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 514 - protein, 265 aa, synthetic construct
  source 1..265: note = Description of Artificial Sequence: Synthetic polypeptide
EVQLVESGGGLVQPGGSLRLSCAASGYSNCAYDMTWYRQAPGKGLEWVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 515 - protein, 265 aa, synthetic construct
  source 1..265: note = Description of Artificial Sequence: Synthetic polypeptide
EVQLLESGGGLVQPGGSLRLSCAASGYSNCAYDMTWYRQAPGKGLEWVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 516 - protein, 265 aa, synthetic construct
  source 1..265: note = Description of Artificial Sequence: Synthetic polypeptide
EVQLVESGGGLVKPGGSLRLSCAASGYSNCAYDMTWYRQAPGKGLEFVSAIHSDGSTRYADSVKGRFFISRDNAKNSLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 517 - protein, 265 aa, synthetic construct
  source 1..265: note = Description of Artificial Sequence: Synthetic polypeptide
EVQLVESGGGLVQPGGSLRLSCAASGYSNCAYDMTWYRQAPGKGLEFVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 518 - protein, 265 aa, synthetic construct
  source 1..265: note = Description of Artificial Sequence: Synthetic polypeptide
EVQLLESGGGLVQPGGSLRLSCAASGYSNCAYDMTWYRQAPGKGLEFVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 519 - protein, 265 aa, synthetic construct
  source 1..265: note = Description of Artificial Sequence: Synthetic polypeptide
EVQLVESGGGLVKPGGSLRLSCAASGYSNCAYDMTWYRQAPGKGREWVSAIHSDGSTRYADSVKGRFFISRDNAKNSLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 520 - protein, 265 aa, synthetic construct
  source 1..265: note = Description of Artificial Sequence: Synthetic polypeptide
EVQLVESGGGLVQPGGSLRLSCAASGYSNCAYDMTWYRQAPGKGREWVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 521 - protein, 265 aa, synthetic construct
  source 1..265: note = Description of Artificial Sequence: Synthetic polypeptide
EVQLLESGGGLVQPGGSLRLSCAASGYSNCAYDMTWYRQAPGKGREWVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 522 - protein, 265 aa, synthetic construct
  source 1..265: note = Description of Artificial Sequence: Synthetic polypeptide
EVQLVESGGGLVKPGGSLRLSCAASGYSNCAYDMTWYRQAPGKELEWVSAIHSDGSTRYADSVKGRFFISRDNAKNSLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 523 - protein, 265 aa, synthetic construct
  source 1..265: note = Description of Artificial Sequence: Synthetic polypeptide
EVQLVESGGGLVQPGGSLRLSCAASGYSNCAYDMTWYRQAPGKELEWVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 524 - protein, 265 aa, synthetic construct
  source 1..265: note = Description of Artificial Sequence: Synthetic polypeptide
EVQLVESGGGLVKPGGSLRLSCAASGYSNCAYDMTWYRQAPGKEREFVSAIHSDGSTRYADSVKGRFFISRDNAKNSLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 525 - protein, 265 aa, synthetic construct
  source 1..265: note = Description of Artificial Sequence: Synthetic polypeptide
EVQLVESGGGLVQPGGSLRLSCAASGYSNCAYDMTWYRQAPGKEREFVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 526 - protein, 265 aa, synthetic construct
  source 1..265: note = Description of Artificial Sequence: Synthetic polypeptide
EVQLLESGGGLVQPGGSLRLSCAASGYSNCAYDMTWYRQAPGKEREFVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 527 - protein, 265 aa, synthetic construct
  source 1..265: note = Description of Artificial Sequence: Synthetic polypeptide
EVQLVESGGGLVQPGGSLRLSCAASGYTQCSYDMTWVRQAPGKGLEFVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEWVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 528 - protein, 265 aa, synthetic construct
  source 1..265: note = Description of Artificial Sequence: Synthetic polypeptide
EVQLVESGGGLVQPGGSLRLSCAASGYTQCSYDMTWVRQAPGKGLEFVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 529 - protein, 265 aa, synthetic construct
  source 1..265: note = Description of Artificial Sequence: Synthetic polypeptide
EVQLVESGGGLVQPGGSLRLSCAASGYTQCSYDMTWYRQAPGKGLEFVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEWVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 530 - protein, 265 aa, synthetic construct
  source 1..265: note = Description of Artificial Sequence: Synthetic polypeptide
EVQLVESGGGLVQPGGSLRLSCAASGYTQCSYDMTWYRQAPGKGLEFVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 531 - protein, 5 aa, synthetic construct
  source 1..5: note = Description of Artificial Sequence: Synthetic 5xHis tag
HHHHH

SEQ ID NO: 532 - protein, 6 aa, synthetic construct
  source 1..6: note = Description of Artificial Sequence: Synthetic 6xHis tag
HHHHHH

SEQ ID NO: 533 - protein, 7 aa, synthetic construct
  source 1..7: note = Description of Artificial Sequence: Synthetic 7xHis tag
HHHHHHH

SEQ ID NO: 534 - protein, 8 aa, synthetic construct
  source 1..8: note = Description of Artificial Sequence: Synthetic 8xHis tag
HHHHHHHH

SEQ ID NO: 535 - protein, 9 aa, synthetic construct
  source 1..9: note = Description of Artificial Sequence: Synthetic 9xHis tag
HHHHHHHHH

SEQ ID NO: 536 - protein, 5 aa, synthetic construct
  source 1..5: note = Description of Artificial Sequence: Synthetic peptide
HWHMY

SEQ ID NO: 537 - protein, 5 aa, synthetic construct
  source 1..5: note = Description of Artificial Sequence: Synthetic peptide
HHHMY

SEQ ID NO: 538 - protein, 5 aa, synthetic construct
  source 1..5: note = Description of Artificial Sequence: Synthetic peptide
HHHHY

SEQ ID NO: 539 - protein, 5 aa, synthetic construct
  source 1..5: note = Description of Artificial Sequence: Synthetic peptide
HWHWH

SEQ ID NO: 540 - protein, 5 aa, synthetic construct
  source 1..5: note = Description of Artificial Sequence: Synthetic peptide
HWHHH

SEQ ID NO: 541 - protein, 9 aa, synthetic construct
  source 1..9: note = Description of Artificial Sequence: Synthetic peptide
HHHHYMHHH

SEQ ID NO: 542 - protein, 5 aa, synthetic construct
  source 1..5: note = Description of Artificial Sequence: Synthetic peptide
HHHHH

SEQ ID NO: 543 - protein, 5 aa, synthetic construct
  source 1..5: note = Description of Artificial Sequence: Synthetic peptide
HWHHH

SEQ ID NO: 544 - protein, 9 aa, synthetic construct
  source 1..9: note = Description of Artificial Sequence: Synthetic peptide
HGHGGGHGH

SEQ ID NO: 545 - protein, 12 aa, synthetic construct
  source 1..12: note = Description of Artificial Sequence: Synthetic peptide
HGHGGGGGGHGH

SEQ ID NO: 546 - DNA, 381 bp, synthetic construct
  source 1..381: mol_type = other DNA; note = Description of Artificial Sequence: Synthetic polynucleotide
gaagtgcagttgctggagagtggtggcggacttgtccaacccggcggtagcctgcgcctcagctgcgctgccagtggctatacacagtgcagttatgacatgacctgggtccgccaagcgcccggcaagggcctggagtgggtttccgctattcactccgatggcagcacccgttatgcagactccgttaagggccgcttcaccatctctcgcgacaactccaagaacaccctgtatcttcagatgaactctctccgcgctgaggacactgctgtgtattactgtaaaaccgatccactgcactgtcgtgcgcacgggggctcatggtactctgtcagagctaactattgggggcagggcaccctggtgactgtgtcctct

SEQ ID NO: 547 - DNA, 381 bp, synthetic construct
  source 1..381: mol_type = other DNA; note = Description of Artificial Sequence: Synthetic polynucleotide
gaggtgcagcttctggagagcggtggcggtctggtacaacccggaggtagtctgcgcctgtcctgtgcggcttccggctacacccaatgttcctatgacatgacctgggttcgccaggcccccggtaagggcctggaatttgtcagcgctatccatagtgatggaagcaccagatatgccgacagtgttaagggccgcttcaccatcagcagagataatagcaagaacaccctttacctccagatgaactctttgcgtgccgaggataccgccgtctattactgtaagaccgatccgctgcattgccgtgcacatggcgggtcctggtacagtgttcgcgccaactattggggccagggaacactggtgactgtcagcagt

SEQ ID NO: 548 - DNA, 381 bp, synthetic construct
  source 1..381: mol_type = other DNA; note = Description of Artificial Sequence: Synthetic polynucleotide
gaagtgcaactggtggagagcggtggaggtttggttcagcctggaggctccctgcgcctttcctgtgctgcgagcgggtatacacagtgcagctatgacatgacctgggtgaggcaggcaccgggcaaaggtttggagtgggtgtcagcgatccattctgatggcagcacaaggtacgcggatagcgttaagggcaggtttttcatttcccgcgacaactctaagaacacactctatctccagatgaactcacttcgcgccgaggataccgccgtgtattactgcaagacagaccctctgcactgtcgggctcacggcggtagctggtattccgtccgtgctaactactggggccagggcactctcgtgaccgtgtcatcc

SEQ ID NO: 549 - DNA, 381 bp, synthetic construct
  source 1..381: mol_type = other DNA; note = Description of Artificial Sequence: Synthetic polynucleotide
gaggtgcagctggtggagtctgggggaggcctggtacagcctgggggcagtctgcgcctgagctgcgcggccagtggctatactcagtgctcctacgacatgacttgggtaaggcaggcccccggcaaaggtttggagtttgtttccgcaattcatagcgacggctccacacgctacgccgattccgtcaaaggtcggtttttcatctcccgtgataacagcaaaaacactctgtaccttcagatgaactccctccgcgcagaggataccgccgtttattactgtaaaactgaccccctccactgccgcgctcacggcgggagctggtatagtgttcgcgccaactactggggccagggcaccttggtgaccgtctccagt

SEQ ID NO: 550 - DNA, 378 bp, synthetic construct
  source 1..378: mol_type = other DNA; note = Description of Artificial Sequence: Synthetic polynucleotide
gaggtgcagttgctggaaagcgggggtggcctggtccagccgggaggttccctgcgcctttcctgcgccgcttccggctacacccattcaagttactgtatgggctgggttcgtcaggcacctggcaaaggtctcgaatgggtgagcgctatcgacgtggacggaagcaccacttatgctgattccgtgaagggtaggttcaccatttcccgcgacaactccaagaacactctttatctgcaaatgaatagcctccgcgctgaggacaccgccgtatattactgcgctgcggagtttgccgactgctctagcaactactttctgccccctggggccgtaaggtattggggccagggcaccttggtgactgtgtcctcc

SEQ ID NO: 551 - DNA, 378 bp, synthetic construct
  source 1..378: mol_type = other DNA; note = Description of Artificial Sequence: Synthetic polynucleotide
gaggtgcagctgctcgaatccgggggcggactggtgcagcctggtggcagcctgcgtctgtcctgtgctgcctctggttatacccattccagctactgtatgggttgggtgcgccaagcccctggaaaggggcttgaaggcgtctccgccattgatgtcgatggcagtaccacttacgctgactccgtgaagggacgcttcactatcagccgcgacaactccaagaacacgctctatctccagatgaactctctccgcgcagaagacaccgcagtgtactattgtgccgcagagtttgccgattgcagctcaaactacttcctgcccccaggggcagtgcgttattggggccagggaacacttgttactgtttcttct

SEQ ID NO: 552 - DNA, 378 bp, synthetic construct
  source 1..378: mol_type = other DNA; note = Description of Artificial Sequence: Synthetic polynucleotide
gaggttcagttggtggaatctggcgggggcctcgtgcagcccggaggctccctcaggctttcctgtgctgcctctgggtacacacactctagttattgcatgggctgggtgcgccaagcaccgggcaagggtctggagtgggtgagcgccattgatgtggacggttctactacctatgccgatagcgtgaaggggcgtttcacaatcagccgcgacaactccaaaaacaccctgtatctccagatgaatagtcttcgtgccgaagacaccgcagtgtactattgtgcggcagagttcgcggactgctctagcaactattttctgcccccaggagccgtccgctactggggacagggcaccctggtcaccgtgtcttcc

SEQ ID NO: 553 - DNA, 378 bp, synthetic construct
  source 1..378: mol_type = other DNA; note = Description of Artificial Sequence: Synthetic polynucleotide
gaggtccagctggtggagagtgggggcggactggtacagccaggtggcagtcttcgcctctcttgcgctgcctccggctacactcacagctcttattgcatgggttgggtgagacaggcccctggtaaaggtcttgagggcgtgagtgccatcgacgtggatggttccactacctacgctgatagtgtgaagggccgcttcactatctcacgtgataactctaagaatactctgtatcttcaaatgaacagcctccgcgcagaggacaccgcagtctactattgtgcagccgagtttgctgactgtagctccaactacttccttccgcccggtgcagtgcgctattgggggcaaggcactctggtgacagtctcctcc

SEQ ID NO: 554 - DNA, 381 bp, synthetic construct
  source 1..381: mol_type = other DNA; note = Description of Artificial Sequence: Synthetic polynucleotide
gaggtgcagctggttgaatccgggggtggcctcgtgcagcccggaggctctctgcgcctgtcctgcgcagcctccggctatacccagtgttcctatgatatgacgtgggtcagacaagctcctggcaaaggccgcgagtttgtttccgctattcactctgacggctccacccggtacgcggatagcgttaagggtcgctttttcatcagccgggataactcaaagaacactctgtacttgcagatgaactctcttcgcgctgaggatacggcggtttattactgcaaaactgatcctctccactgtcgcgcccacggcggatcttggtatagcgttcgcgctaattactgggggcaaggaactctggtgactgtttcctcc

SEQ ID NO: 555 - DNA, 381 bp, synthetic construct
  source 1..381: mol_type = other DNA; note = Description of Artificial Sequence: Synthetic polynucleotide
gaggtgcagttgcaggagtccggcggagggtccgtccaggccggaggcagtctgcgcctgtcctgtgccgcttccggctactcccaatgttcttacgacatgacttggtatcgccaggctccgggcaaggagcgcgagttcgtgagcgccatccactcagacggctccacccgttacgccgactcagtcaagggtcgcttctttatctcccaggacaacgcaaagaacaccgtctatctccagatgaactccctgaagccagaagacacagctatgtattactgtaaaaccgacccactgcattgcagggctcatggtggctcctggtactctgtacgcgcaaactactggggtcagggcacccaggtgaccgtgagttcc

SEQ ID NO: 556 - DNA, 795 bp, synthetic construct
  source 1..795: mol_type = other DNA; note = Description of Artificial Sequence: Synthetic polynucleotide
gaagtccagcttgttgaatctgggggaggcctggtgcagcctgggggctccctgcgcctctcctgtgctgccagcgggtacacccaatgttcctatgatatgacctgggtccgccaggcaccaggcaagggattggaatttgtgagcgcaatccattctgacggcagtactcgttacgctgactccgtaaagggcagattctttatctctcgtgacaacagcaaaaacactctgtatctgcaaatgaactctctccgcgccgaagacactgcggtctattactgcaagaccgatcccctccattgtcgtgcccacgggggaagttggtactcagtccgcgcaaattattggggccagggcactctcgtgaccgtctccagcgggggaggttccgaggtgcagctcgtggagagcggtgggggcctggtccagcctggcggaagcctgcgcctgtcatgcgctgcgtctggttacacacacagctcctattgtatgggatgggtgcggcaggcccccggtaaaggactggaatgggttagcgccatcgacgtggacggctctacgacatacgccgactccgtcaagggccgctttactatcagccgtgataacagcaagaacactctgtacctgcaaatgaactctctccgcgccgaggacaccgctgtgtattactgtgccgcagagttcgcagactgctcttccaattatttcctgcctcccggagccgtccgttattggggtcagggcactctggttactgtgtcctctgcttcccatcaccatcatcaccac

SEQ ID NO: 557 - DNA, 795 bp, synthetic construct
  source 1..795: mol_type = other DNA; note = Description of Artificial Sequence: Synthetic polynucleotide
gaggtgcagctggttgagtctggcggaggtctggtgcagcccggcggttctctgcgcctgtcttgcgccgcgagcggctacacgcaatgcagctacgatatgacctgggttcgtcaggcccccggcaagggcttggagttcgtcagcgcgatccatagcgacggctccacaagatatgctgatagcgtgaagggccgcttctttattagtagagacaactctaagaacacactctacctccagatgaatagtctccgcgcagaggacacggcggtgtactattgtaagaccgatcctctgcattgcagagcccacggtggctcctggtacagcgtgcgggctaactattgggggcaaggcaccctggttactgtctctagcggtggaggtagcgaagtccaacttgtcgagtccgggggaggtctggtccagcccggtgggtctcttaggctctcctgcgctgcatccggctacacccatagctcatattgtatgggctgggtccgccaagcccctgggaaggggctggaaggtgtcagcgccattgatgtcgatggcagcacaacttacgctgatagtgtgaagggtcgcttcactatctccagagacaattcaaagaacaccttgtacctccagatgaactctcttcgcgcggaagacaccgccgtctattactgcgccgctgagttcgcggactgtagctccaattatttcctgccacccggcgctgtgcggtattggggccagggtactctggtgactgtgtctagtgcctctcaccatcaccatcatcac

SEQ ID NO: 558 - DNA, 795 bp, synthetic construct
  source 1..795: mol_type = other DNA; note = Description of Artificial Sequence: Synthetic polynucleotide
gaggtgcagctggtggaatctggaggcggtctcgtccagccagggggctctctgcgtctgtcttgcgctgcgtctggatatacccagtgctcttacgacatgacatggtatcggcaggctcccggcaagggacttgagttcgtctctgccatccacagcgatggttccacacgctacgcagattccgtgaagggacgttttttcatcagccgtgacaatagcaagaacactctgtatcttcaaatgaactccctccgcgcggaagacaccgccgtgtattactgtaaaaccgatcctctgcactgtagggcacatgggggctcttggtattctgttcgtgctaattattggggccagggcaccctggtcaccgtttccagcggcggaggtagtgaggtccagctcgtcgaatccggcggtggcctcgtgcagcctggcgggtccctccgcctgtcctgcgctgcaagcggttatacgcacagctcctattgcatgggctgggtgcgtcaagctcctggcaaaggcctggagtgggtcagcgctattgacgtggacgggagtactacctacgccgattcagtgaagggtagattcaccatctcacgtgacaactctaagaacacactgtacttgcagatgaatagcctgagggctgaagacactgctgtgtactattgcgctgccgagttcgccgattgtagctccaactacttcctgcccccaggagcggtccgttactggggccaaggcaccctcgtgactgtctcttcagccagtcatcaccatcatcaccac

SEQ ID NO: 559 - DNA, 795 bp, synthetic construct
  source 1..795: mol_type = other DNA; note = Description of Artificial Sequence: Synthetic polynucleotide
gaggtccagctggtggagagtgggggtgggctcgttcagcctgggggctctctgaggctgagctgtgcagcctccggctatacccagtgttcttacgacatgacgtggtataggcaggcaccaggcaaaggactggagtttgtgagtgctatccactccgacggttccacccggtatgctgactctgttaagggcagattctttattagccgggacaacagcaaaaacaccctttacctccagatgaactctctgagggcagaggacacggcagtgtattactgtaaaacagaccctcttcactgccgcgcacatggaggctcttggtacagtgtgagggctaattattggggtcagggcaccctcgtcacagtctcttcagggggtggatctgaggtgcagttggtcgagagtgggggtggcctggtgcagcctggcggtagcctgcgcctgagctgcgcggcctctgggtacacccactcctcttactgtatgggctgggtgaggcaggctcctggaaaggggctggagggcgtgtccgctattgacgtagatggctccactacctacgccgacagcgtaaaaggtcgtttcacaatctcccgcgacaactccaagaacaccctctacttgcagatgaactccctgcgggccgaagacacagctgtttattactgcgccgctgagttcgcagactgttccagtaattacttcctgccccctggtgccgtgcgttactggggccagggcaccctcgtaaccgtcagctccgctagtcaccatcaccaccaccat

SEQ ID NO: 560 - protein, 8 aa, synthetic construct
  source 1..8: note = Description of Artificial Sequence: Synthetic peptide
ASHHHHHH

SEQ ID NO: 561 - protein, 265 aa, synthetic construct
  source 1..265: note = Description of Artificial Sequence: Synthetic polypeptide
EVQLLESGGGLVQPGGSLRLSCAASGYSNCAYDMTWYRQAPGKELEWVSAIHSDGSTRYADSVKGRFFISRDNSKNTLYLQMNSLRAEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSSASHHHHHH

SEQ ID NO: 562 - protein, 800 aa, synthetic construct
  source 1..800: note = Description of Artificial Sequence: Synthetic polypeptide
  VARIANT 1..20: note = SITE - This region may encompass 1-20 residues
  VARIANT 21..40: note = SITE - This region may encompass 1-20 residues
  VARIANT 41..60: note = SITE - This region may encompass 1-20 residues
  VARIANT 61..80: note = SITE - This region may encompass 1-20 residues
  VARIANT 81..100: note = SITE - This region may encompass 1-20 residues
  VARIANT 101..120: note = SITE - This region may encompass 1-20 residues
  VARIANT 121..140: note = SITE - This region may encompass 1-20 residues
  VARIANT 141..160: note = SITE - This region may encompass 1-20 residues
  VARIANT 161..180: note = SITE - This region may encompass 1-20 residues
  VARIANT 181..200: note = SITE - This region may encompass 1-20 residues
  VARIANT 201..220: note = SITE - This region may encompass 1-20 residues
  VARIANT 221..240: note = SITE - This region may encompass 1-20 residues
  VARIANT 241..260: note = SITE - This region may encompass 1-20 residues
  VARIANT 261..280: note = SITE - This region may encompass 1-20 residues
  VARIANT 281..300: note = SITE - This region may encompass 1-20 residues
  VARIANT 301..320: note = SITE - This region may encompass 1-20 residues
  VARIANT 321..340: note = SITE - This region may encompass 1-20 residues
  VARIANT 341..360: note = SITE - This region may encompass 1-20 residues
  VARIANT 361..380: note = SITE - This region may encompass 1-20 residues
  VARIANT 381..400: note = SITE - This region may encompass 1-20 residues
  VARIANT 401..420: note = SITE - This region may encompass 1-20 residues
  VARIANT 421..440: note = SITE - This region may encompass 1-20 residues
  VARIANT 441..460: note = SITE - This region may encompass 1-20 residues
  VARIANT 461..480: note = SITE - This region may encompass 1-20 residues
  VARIANT 481..500: note = SITE - This region may encompass 1-20 residues
  VARIANT 501..520: note = SITE - This region may encompass 1-20 residues
  VARIANT 521..540: note = SITE - This region may encompass 1-20 residues
  VARIANT 541..560: note = SITE - This region may encompass 1-20 residues
  VARIANT 561..580: note = SITE - This region may encompass 1-20 residues
  VARIANT 581..600: note = SITE - This region may encompass 1-20 residues
  VARIANT 601..620: note = SITE - This region may encompass 1-20 residues
  VARIANT 621..640: note = SITE - This region may encompass 1-20 residues
  VARIANT 641..660: note = SITE - This region may encompass 1-20 residues
  VARIANT 661..680: note = SITE - This region may encompass 1-20 residues
  VARIANT 681..700: note = SITE - This region may encompass 1-20 residues
  VARIANT 701..720: note = SITE - This region may encompass 1-20 residues
  VARIANT 721..740: note = SITE - This region may encompass 1-20 residues
  VARIANT 741..760: note = SITE - This region may encompass 1-20 residues
  VARIANT 761..780: note = SITE - This region may encompass 1-20 residues
  VARIANT 781..800: note = SITE - This region may encompass 1-20 residues
  VARIANT 1..800: note = SITE - This sequence may encompass 1-20 (Gly)m(Ser)o repeating units, wherein m = 1 to 20, o = 1 to 20
  REGION 1..800: note = See specification as filed for detailed description of substitutions and preferred embodiments
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSS

SEQ ID NO: 563 - protein, 100 aa, synthetic construct
  source 1..100: note = Description of Artificial Sequence: Synthetic polypeptide
  VARIANT 1..100: note = SITE - This sequence may encompass 1-20 Gly Ser Gly Gly Ser repeating units
  REGION 1..100: note = See specification as filed for detailed description of substitutions and preferred embodiments
GSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGS

SEQ ID NO: 564 - protein, 1200 aa, synthetic construct
  source 1..1200: note = Description of Artificial Sequence: Synthetic polypeptide
  VARIANT 1..20: note = SITE - This region may encompass 1-20 residues
  VARIANT 21..40: note = SITE - This region may encompass 1-20 residues
  VARIANT 41..60: note = SITE - This region may encompass 1-20 residues
  VARIANT 61..80: note = SITE - This region may encompass 1-20 residues
  VARIANT 81..100: note = SITE - This region may encompass 1-20 residues
  VARIANT 101..120: note = SITE - This region may encompass 1-20 residues
  VARIANT 121..140: note = SITE - This region may encompass 1-20 residues
  VARIANT 141..160: note = SITE - This region may encompass 1-20 residues
  VARIANT 161..180: note = SITE - This region may encompass 1-20 residues
  VARIANT 181..200: note = SITE - This region may encompass 1-20 residues
  VARIANT 201..220: note = SITE - This region may encompass 1-20 residues
  VARIANT 221..240: note = SITE - This region may encompass 1-20 residues
  VARIANT 241..260: note = SITE - This region may encompass 1-20 residues
  VARIANT 261..280: note = SITE - This region may encompass 1-20 residues
  VARIANT 281..300: note = SITE - This region may encompass 1-20 residues
  VARIANT 301..320: note = SITE - This region may encompass 1-20 residues
  VARIANT 321..340: note = SITE - This region may encompass 1-20 residues
  VARIANT 341..360: note = SITE - This region may encompass 1-20 residues
  VARIANT 361..380: note = SITE - This region may encompass 1-20 residues
  VARIANT 381..400: note = SITE - This region may encompass 1-20 residues
  VARIANT 401..420: note = SITE - This region may encompass 1-20 residues
  VARIANT 421..440: note = SITE - This region may encompass 1-20 residues
  VARIANT 441..460: note = SITE - This region may encompass 1-20 residues
  VARIANT 461..480: note = SITE - This region may encompass 1-20 residues
  VARIANT 481..500: note = SITE - This region may encompass 1-20 residues
  VARIANT 501..520: note = SITE - This region may encompass 1-20 residues
  VARIANT 521..540: note = SITE - This region may encompass 1-20 residues
  VARIANT 541..560: note = SITE - This region may encompass 1-20 residues
  VARIANT 561..580: note = SITE - This region may encompass 1-20 residues
  VARIANT 581..600: note = SITE - This region may encompass 1-20 residues
  VARIANT 601..620: note = SITE - This region may encompass 1-20 residues
  VARIANT 621..640: note = SITE - This region may encompass 1-20 residues
  VARIANT 641..660: note = SITE - This region may encompass 1-20 residues
  VARIANT 661..680: note = SITE - This region may encompass 1-20 residues
  VARIANT 681..700: note = SITE - This region may encompass 1-20 residues
  VARIANT 701..720: note = SITE - This region may encompass 1-20 residues
  VARIANT 721..740: note = SITE - This region may encompass 1-20 residues
  VARIANT 741..760: note = SITE - This region may encompass 1-20 residues
  VARIANT 761..780: note = SITE - This region may encompass 1-20 residues
  VARIANT 781..800: note = SITE - This region may encompass 1-20 residues
  VARIANT 801..820: note = SITE - This region may encompass 1-20 residues
  VARIANT 821..840: note = SITE - This region may encompass 1-20 residues
  VARIANT 841..860: note = SITE - This region may encompass 1-20 residues
  VARIANT 861..880: note = SITE - This region may encompass 1-20 residues
  VARIANT 881..900: note = SITE - This region may encompass 1-20 residues
  VARIANT 901..920: note = SITE - This region may encompass 1-20 residues
  VARIANT 921..940: note = SITE - This region may encompass 1-20 residues
  VARIANT 941..960: note = SITE - This region may encompass 1-20 residues
  VARIANT 961..980: note = SITE - This region may encompass 1-20 residues
  VARIANT 981..1000: note = SITE - This region may encompass 1-20 residues
  VARIANT 1001..1020: note = SITE - This region may encompass 1-20 residues
  VARIANT 1021..1040: note = SITE - This region may encompass 1-20 residues
  VARIANT 1041..1060: note = SITE - This region may encompass 1-20 residues
  VARIANT 1061..1080: note = SITE - This region may encompass 1-20 residues
  VARIANT 1081..1100: note = SITE - This region may encompass 1-20 residues
  VARIANT 1101..1120: note = SITE - This region may encompass 1-20 residues
  VARIANT 1121..1140: note = SITE - This region may encompass 1-20 residues
  VARIANT 1141..1160: note = SITE - This region may encompass 1-20 residues
  VARIANT 1161..1180: note = SITE - This region may encompass 1-20 residues
  VARIANT 1181..1200: note = SITE - This region may encompass 1-20 residues
  VARIANT 1..1200: note = SITE - This sequence may encompass 1-20 (Gly)m(Ser)o(Gly)m repeating units, wherein m = 1 to 20, o = 1 to 20
  REGION 1..1200: note = See specification as filed for detailed description of substitutions and preferred embodiments
GGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGG

SEQ ID NO: 565 - protein, 2000 aa, synthetic construct
  source 1..2000: note = Description of Artificial Sequence: Synthetic polypeptide
  VARIANT 1..20: note = SITE - This region may encompass 1-20 residues
  VARIANT 21..40: note = SITE - This region may encompass 1-20 residues
  VARIANT 41..60: note = SITE - This region may encompass 1-20 residues
  VARIANT 61..80: note = SITE - This region may encompass 1-20 residues
  VARIANT 81..100: note = SITE - This region may encompass 1-20 residues
  VARIANT 101..120: note = SITE - This region may encompass 1-20 residues
  VARIANT 121..140: note = SITE - This region may encompass 1-20 residues
  VARIANT 141..160: note = SITE - This region may encompass 1-20 residues
  VARIANT 161..180: note = SITE - This region may encompass 1-20 residues
  VARIANT 181..200: note = SITE - This region may encompass 1-20 residues
  VARIANT 201..220: note = SITE - This region may encompass 1-20 residues
  VARIANT 221..240: note = SITE - This region may encompass 1-20 residues
  VARIANT 241..260: note = SITE - This region may encompass 1-20 residues
  VARIANT 261..280: note = SITE - This region may encompass 1-20 residues
  VARIANT 281..300: note = SITE - This region may encompass 1-20 residues
  VARIANT 301..320: note = SITE - This region may encompass 1-20 residues
  VARIANT 321..340: note = SITE - This region may encompass 1-20 residues
  VARIANT 341..360: note = SITE - This region may encompass 1-20 residues
  VARIANT 361..380: note = SITE - This region may encompass 1-20 residues
  VARIANT 381..400: note = SITE - This region may encompass 1-20 residues
  VARIANT 401..420: note = SITE - This region may encompass 1-20 residues
  VARIANT 421..440: note = SITE - This region may encompass 1-20 residues
  VARIANT 441..460: note = SITE - This region may encompass 1-20 residues
  VARIANT 461..480: note = SITE - This region may encompass 1-20 residues
  VARIANT 481..500: note = SITE - This region may encompass 1-20 residues
  VARIANT 501..520: note = SITE - This region may encompass 1-20 residues
  VARIANT 521..540: note = SITE - This region may encompass 1-20 residues
  VARIANT 541..560: note = SITE - This region may encompass 1-20 residues
  VARIANT 561..580: note = SITE - This region may encompass 1-20 residues
  VARIANT 581..600: note = SITE - This region may encompass 1-20 residues
  VARIANT 601..620: note = SITE - This region may encompass 1-20 residues
  VARIANT 621..640: note = SITE - This region may encompass 1-20 residues
  VARIANT 641..660: note = SITE - This region may encompass 1-20 residues
  VARIANT 661..680: note = SITE - This region may encompass 1-20 residues
  VARIANT 681..700: note = SITE - This region may encompass 1-20 residues
  VARIANT 701..720: note = SITE - This region may encompass 1-20 residues
  VARIANT 721..740: note = SITE - This region may encompass 1-20 residues
  VARIANT 741..760: note = SITE - This region may encompass 1-20 residues
  VARIANT 761..780: note = SITE - This region may encompass 1-20 residues
  VARIANT 781..800: note = SITE - This region may encompass 1-20 residues
  VARIANT 801..820: note = SITE - This region may encompass 1-20 residues
  VARIANT 821..840: note = SITE - This region may encompass 1-20 residues
  VARIANT 841..860: note = SITE - This region may encompass 1-20 residues
  VARIANT 861..880: note = SITE - This region may encompass 1-20 residues
  VARIANT 881..900: note = SITE - This region may encompass 1-20 residues
  VARIANT 901..920: note = SITE - This region may encompass 1-20 residues
  VARIANT 921..940: note = SITE - This region may encompass 1-20 residues
  VARIANT 941..960: note = SITE - This region may encompass 1-20 residues
  VARIANT 961..980: note = SITE - This region may encompass 1-20 residues
  VARIANT 981..1000: note = SITE - This region may encompass 1-20 residues
  VARIANT 1001..1020: note = SITE - This region may encompass 1-20 residues
  VARIANT 1021..1040: note = SITE - This region may encompass 1-20 residues
  VARIANT 1041..1060: note = SITE - This region may encompass 1-20 residues
  VARIANT 1061..1080: note = SITE - This region may encompass 1-20 residues
  VARIANT 1081..1100: note = SITE - This region may encompass 1-20 residues
  VARIANT 1101..1120: note = SITE - This region may encompass 1-20 residues
  VARIANT 1121..1140: note = SITE - This region may encompass 1-20 residues
  VARIANT 1141..1160: note = SITE - This region may encompass 1-20 residues
  VARIANT 1161..1180: note = SITE - This region may encompass 1-20 residues
  VARIANT 1181..1200: note = SITE - This region may encompass 1-20 residues
  VARIANT 1201..1220: note = SITE - This region may encompass 1-20 residues
  VARIANT 1221..1240: note = SITE - This region may encompass 1-20 residues
  VARIANT 1241..1260: note = SITE - This region may encompass 1-20 residues
  VARIANT 1261..1280: note = SITE - This region may encompass 1-20 residues
  VARIANT 1281..1300: note = SITE - This region may encompass 1-20 residues
  VARIANT 1301..1320: note = SITE - This region may encompass 1-20 residues
  VARIANT 1321..1340: note = SITE - This region may encompass 1-20 residues
  VARIANT 1341..1360: note = SITE - This region may encompass 1-20 residues
  VARIANT 1361..1380: note = SITE - This region may encompass 1-20 residues
  VARIANT 1381..1400: note = SITE - This region may encompass 1-20 residues
  VARIANT 1401..1420: note = SITE - This region may encompass 1-20 residues
  VARIANT 1421..1440: note = SITE - This region may encompass 1-20 residues
  VARIANT 1441..1460: note = SITE - This region may encompass 1-20 residues
  VARIANT 1461..1480: note = SITE - This region may encompass 1-20 residues
  VARIANT 1481..1500: note = SITE - This region may encompass 1-20 residues
  VARIANT 1501..1520: note = SITE - This region may encompass 1-20 residues
  VARIANT 1521..1540: note = SITE - This region may encompass 1-20 residues
  VARIANT 1541..1560: note = SITE - This region may encompass 1-20 residues
  VARIANT 1561..1580: note = SITE - This region may encompass 1-20 residues
  VARIANT 1581..1600: note = SITE - This region may encompass 1-20 residues
  VARIANT 1601..1620: note = SITE - This region may encompass 1-20 residues
  VARIANT 1621..1640: note = SITE - This region may encompass 1-20 residues
  VARIANT 1641..1660: note = SITE - This region may encompass 1-20 residues
  VARIANT 1661..1680: note = SITE - This region may encompass 1-20 residues
  VARIANT 1681..1700: note = SITE - This region may encompass 1-20 residues
  VARIANT 1701..1720: note = SITE - This region may encompass 1-20 residues
  VARIANT 1721..1740: note = SITE - This region may encompass 1-20 residues
  VARIANT 1741..1760: note = SITE - This region may encompass 1-20 residues
  VARIANT 1761..1780: note = SITE - This region may encompass 1-20 residues
  VARIANT 1781..1800: note = SITE - This region may encompass 1-20 residues
  VARIANT 1801..1820: note = SITE - This region may encompass 1-20 residues
  VARIANT 1821..1840: note = SITE - This region may encompass 1-20 residues
  VARIANT 1841..1860: note = SITE - This region may encompass 1-20 residues
  VARIANT 1861..1880: note = SITE - This region may encompass 1-20 residues
  VARIANT 1881..1900: note = SITE - This region may encompass 1-20 residues
  VARIANT 1901..1920: note = SITE - This region may encompass 1-20 residues
  VARIANT 1921..1940: note = SITE - This region may encompass 1-20 residues
  VARIANT 1941..1960: note = SITE - This region may encompass 1-20 residues
  VARIANT 1961..1980: note = SITE - This region may encompass 1-20 residues
  VARIANT 1981..2000: note = SITE - This region may encompass 1-20 residues
  VARIANT 1..2000: note = SITE - This sequence may encompass 1-20 (Gly)m(Ser)o(Gly)m(Ser)o(Gly)m repeating units, wherein m = 1 to 20, o = 1 to 20
  REGION 1..2000: note = See specification as filed for detailed description of substitutions and preferred embodiments
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGG

SEQ ID NO: 566 - protein, 480 aa, synthetic construct
  source 1..480: note = Description of Artificial Sequence: Synthetic polypeptide
  VARIANT 5..24: note = SITE - This region may encompass 1-20 residues
  VARIANT 29..48: note = SITE - This region may encompass 1-20 residues
  VARIANT 53..72: note = SITE - This region may encompass 1-20 residues
  VARIANT 77..96: note = SITE - This region may encompass 1-20 residues
  VARIANT 101..120: note = SITE - This region may encompass 1-20 residues
  VARIANT 125..144: note = SITE - This region may encompass 1-20 residues
  VARIANT 149..168: note = SITE - This region may encompass 1-20 residues
  VARIANT 173..192: note = SITE - This region may encompass 1-20 residues
  VARIANT 197..216: note = SITE - This region may encompass 1-20 residues
  VARIANT 221..240: note = SITE - This region may encompass 1-20 residues
  VARIANT 245..264: note = SITE - This region may encompass 1-20 residues
  VARIANT 269..288: note = SITE - This region may encompass 1-20 residues
  VARIANT 293..312: note = SITE - This region may encompass 1-20 residues
  VARIANT 317..336: note = SITE - This region may encompass 1-20 residues
  VARIANT 341..360: note = SITE - This region may encompass 1-20 residues
  VARIANT 365..384: note = SITE - This region may encompass 1-20 residues
  VARIANT 389..408: note = SITE - This region may encompass 1-20 residues
  VARIANT 413..432: note = SITE - This region may encompass 1-20 residues
  VARIANT 437..456: note = SITE - This region may encompass 1-20 residues
  VARIANT 461..480: note = SITE - This region may encompass 1-20 residues
  VARIANT 1..480: note = SITE - This sequence may encompass 1-20 (Gly)(Ser)(Gly)(Gly)(Ser)m repeating units, wherein m = 1 to 20
  REGION 1..480: note = See specification as filed for detailed description of substitutions and preferred embodiments
GSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSS

SEQ ID NO: 567 - protein, 480 aa, synthetic construct
  source 1..480: note = Description of Artificial Sequence: Synthetic polypeptide
  VARIANT 4..23: note = SITE - This region may encompass 1-20 residues
  VARIANT 28..47: note = SITE - This region may encompass 1-20 residues
  VARIANT 52..71: note = SITE - This region may encompass 1-20 residues
  VARIANT 76..95: note = SITE - This region may encompass 1-20 residues
  VARIANT 100..119: note = SITE - This region may encompass 1-20 residues
  VARIANT 124..143: note = SITE - This region may encompass 1-20 residues
  VARIANT 148..167: note = SITE - This region may encompass 1-20 residues
  VARIANT 172..191: note = SITE - This region may encompass 1-20 residues
  VARIANT 196..215: note = SITE - This region may encompass 1-20 residues
  VARIANT 220..239: note = SITE - This region may encompass 1-20 residues
  VARIANT 244..263: note = SITE - This region may encompass 1-20 residues
  VARIANT 268..287: note = SITE - This region may encompass 1-20 residues
  VARIANT 292..311: note = SITE - This region may encompass 1-20 residues
  VARIANT 316..335: note = SITE - This region may encompass 1-20 residues
  VARIANT 340..359: note = SITE - This region may encompass 1-20 residues
  VARIANT 364..383: note = SITE - This region may encompass 1-20 residues
  VARIANT 388..407: note = SITE - This region may encompass 1-20 residues
  VARIANT 412..431: note = SITE - This region may encompass 1-20 residues
  VARIANT 436..455: note = SITE - This region may encompass 1-20 residues
  VARIANT 460..479: note = SITE - This region may encompass 1-20 residues
  VARIANT 1..480: note = SITE - This sequence may encompass 1-20 (Gly)(Ser)(Gly)(Ser)m(Gly) repeating units, wherein m = 1 to 20
  REGION 1..480: note = See specification as filed for detailed description of substitutions and preferred embodiments
GSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSG

SEQ ID NO: 568 - protein, 460 aa, synthetic construct
  source 1..460: note = Description of Artificial Sequence: Synthetic polypeptide
  VARIANT 4..23: note = SITE - This region may encompass 1-20 residues
  VARIANT 27..46: note = SITE - This region may encompass 1-20 residues
  VARIANT 50..69: note = SITE - This region may encompass 1-20 residues
  VARIANT 73..92: note = SITE - This region may encompass 1-20 residues
  VARIANT 96..115: note = SITE - This region may encompass 1-20 residues
  VARIANT 119..138: note = SITE - This region may encompass 1-20 residues
  VARIANT 142..161: note = SITE - This region may encompass 1-20 residues
  VARIANT 165..184: note = SITE - This region may encompass 1-20 residues
  VARIANT 188..207: note = SITE - This region may encompass 1-20 residues
  VARIANT 211..230: note = SITE - This region may encompass 1-20 residues
  VARIANT 234..253: note = SITE - This region may encompass 1-20 residues
  VARIANT 257..276: note = SITE - This region may encompass 1-20 residues
  VARIANT 280..299: note = SITE - This region may encompass 1-20 residues
  VARIANT 303..322: note = SITE - This region may encompass 1-20 residues
  VARIANT 326..345: note = SITE - This region may encompass 1-20 residues
  VARIANT 349..368: note = SITE - This region may encompass 1-20 residues
  VARIANT 372..391: note = SITE - This region may encompass 1-20 residues
  VARIANT 395..414: note = SITE - This region may encompass 1-20 residues
  VARIANT 418..437: note = SITE - This region may encompass 1-20 residues
  VARIANT 441..460: note = SITE - This region may encompass 1-20 residues
  VARIANT 1..460: note = SITE - This sequence may encompass 1-20 (Gly)(Gly)(Gly)(Ser)m repeating units, wherein m = 1 to 20
  REGION 1..460: note = See specification as filed for detailed description of substitutions and preferred embodiments
GGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSS

SEQ ID NO: 569 - protein, 6 aa, synthetic construct
  source 1..6: note = Description of Artificial Sequence: Synthetic His tag
  VARIANT 1..6: note = SITE - This sequence may encompass 3-6 residues
HHHHHH